SEQUENCE LISTING (72 sequences)

SEQ ID NO: 1 - protein, 15 aa, synthetic construct
  source 1..15: note = Description of Artificial Sequence: Synthetic peptide
RASKGVSTSGYSYLH

SEQ ID NO: 2 - protein, 7 aa, synthetic construct
  source 1..7: note = Description of Artificial Sequence: Synthetic peptide
LASYLES

SEQ ID NO: 3 - protein, 9 aa, synthetic construct
  source 1..9: note = Description of Artificial Sequence: Synthetic peptide
QHSRDLPLT

SEQ ID NO: 4 - protein, 111 aa, synthetic construct [sequence omitted]

SEQ ID NO: 5 - protein, 218 aa, synthetic construct
  source 1..218: note = Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
  source 1..5: note = Description of Artificial Sequence: Synthetic peptide
NYYMY

SEQ ID NO: 7 - protein, 17 aa, synthetic construct
  source 1..17: note = Description of Artificial Sequence: Synthetic peptide
GINPSNGGTNFNEKFKN

SEQ ID NO: 8 - protein, 11 aa, synthetic construct
  source 1..11: note = Description of Artificial Sequence: Synthetic peptide
RDYRFDMGFDY

SEQ ID NO: 9 - protein, 120 aa, synthetic construct [sequence omitted]

SEQ ID NO: 10 - protein, 447 aa, synthetic construct
  source 1..447: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
  source 1..11: note = Description of Artificial Sequence: Synthetic peptide
RASQSVSSYLA

SEQ ID NO: 12 - protein, 7 aa, synthetic construct
  source 1..7: note = Description of Artificial Sequence: Synthetic peptide
DASNRAT

SEQ ID NO: 13 - protein, 9 aa, synthetic construct
  source 1..9: note = Description of Artificial Sequence: Synthetic peptide
QQSSNWPRT

SEQ ID NO: 14 - protein, 107 aa, synthetic construct [sequence omitted]

SEQ ID NO: 15 - protein, 214 aa, synthetic construct
  source 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  source 1..5: note = Description of Artificial Sequence: Synthetic peptide
NSGMH

SEQ ID NO: 17 - protein, 17 aa, synthetic construct
  source 1..17: note = Description of Artificial Sequence: Synthetic peptide
VIWYDGSKRYYADSVKG

SEQ ID NO: 18 - protein, 4 aa, synthetic construct
  source 1..4: note = Description of Artificial Sequence: Synthetic peptide
NDDY

SEQ ID NO: 19 - protein, 113 aa, synthetic construct [sequence omitted]

SEQ ID NO: 20 - protein, 440 aa, synthetic construct
  source 1..440: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 18632 bp, synthetic construct
  source 1..18632: mol_type = other DNA; note = Description of Artificial Sequence: Synthetic polynucleotide
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 22 - protein, 532 aa, synthetic construct
  source 1..532: note = Description of Artificial Sequence: Synthetic polypeptide
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 328 aa, Homo sapiens
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 24 - protein, 7 aa, synthetic construct
  source 1..7: note = Description of Artificial Sequence: Synthetic peptide
GGGGGGS

SEQ ID NO: 25 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 26 - DNA, 1599 bp, synthetic construct
  source 1..1599: mol_type = other DNA; note = Description of Artificial Sequence: Synthetic polynucleotide
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 27 - DNA, 984 bp, Homo sapiens
  source 1..984: mol_type = other DNA
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 28 - DNA, 21 bp, synthetic construct
  source 1..21: mol_type = other DNA; note = Description of Artificial Sequence: Synthetic oligonucleotide
ggtggcggtggcggcggatct

SEQ ID NO: 29 - DNA, 594 bp, Homo sapiens
  source 1..594: mol_type = other DNA
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 30 - DNA, 1641 bp, synthetic construct
  source 1..1641: mol_type = other DNA; note = Description of Artificial Sequence: Synthetic polynucleotide
atgtctgcacttctgatcctagctcttgttggagctgcagttgctgactacaaagacgatgacgacaagctttgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctggatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgctgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatcctctagaggtagtggatccggtggcagtggaggttctggatctggtaagcttagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 31 - DNA, 20210 bp, synthetic construct
  source 1..20210: mol_type = other DNA; note = Description of Artificial Sequence: Synthetic polynucleotide
tcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaaccgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatgatccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 32 - DNA, 65 bp, synthetic construct
  source 1..65: mol_type = other DNA; note = Description of Artificial Sequence: Synthetic primer
gcaccgagttcccccccgcggttagaaaaaatacgggtagaaccgccaccatgggtcaccagcag

SEQ ID NO: 33 - DNA, 37 bp, synthetic construct
  source 1..37: mol_type = other DNA; note = Description of Artificial Sequence: Synthetic primer
agttggaccttgggtccgcggattaggaagcattcag

SEQ ID NO: 34 - DNA, 65 bp, synthetic construct
  source 1..65: mol_type = other DNA; note = Description of Artificial Sequence: Synthetic primer
gcaccgagttcccccccgcggttagaaaaaatacgggtagaaccgccaccatgtacagcatgcag

SEQ ID NO: 35 - DNA, 37 bp, synthetic construct
  source 1..37: mol_type = other DNA; note = Description of Artificial Sequence: Synthetic primer
agttggaccttgggtccgcggattattgagggcttgt

SEQ ID NO: 36 - DNA, 18 bp, synthetic construct
  source 1..18: mol_type = other DNA; note = Description of Artificial Sequence: Synthetic primer
tcgatcgaggaaatcagg

SEQ ID NO: 37 - DNA, 17 bp, synthetic construct
  source 1..17: mol_type = other DNA; note = Description of Artificial Sequence: Synthetic primer
gtacagcccaattgtcc

SEQ ID NO: 38 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - protein, 510 aa, synthetic construct
  source 1..510: note = Description of Artificial Sequence: Synthetic polypeptide
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 40 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 42 - protein, 532 aa, synthetic construct
  source 1..532: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 43 - protein, 510 aa, synthetic construct
  source 1..510: note = Description of Artificial Sequence: Synthetic polypeptide
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 44 - protein, 5 aa, synthetic construct
  VARIANT 4: note = replace with "Arg" or "Asp" or "Cys" or "Glu" or "Gly" or "Gln" or "Ser" or "Tyr" or "Asn" or "His" or "Ile" or "Leu" or "Lys" or "Met" or "Phe" or "Thr" or "Trp" or "Val"
  source 1..5: note = Description of Artificial Sequence: Synthetic peptide; note = Variant residues given in the sequence have no preference with respect to those in the annotations for variant positions
VPGAG

SEQ ID NO: 45 - protein, 10 aa, synthetic construct
  VARIANT 4: note = replace with "Arg" or "Asp" or "Cys" or "Glu" or "Gly" or "Gln" or "Ser" or "Tyr" or "Asn" or "His" or "Ile" or "Leu" or "Lys" or "Met" or "Phe" or "Thr" or "Trp" or "Val"
  VARIANT 9: note = replace with "Arg" or "Asp" or "Cys" or "Glu" or "Gly" or "Gln" or "Ser" or "Tyr" or "Asn" or "His" or "Ile" or "Leu" or "Lys" or "Met" or "Phe" or "Thr" or "Trp" or "Val"
  source 1..10: note = Description of Artificial Sequence: Synthetic peptide; note = Variant residues given in the sequence have no preference with respect to those in the annotations for variant positions
VPGAGVPGAG

SEQ ID NO: 46 - protein, 5 aa, synthetic construct
  source 1..5: note = Description of Artificial Sequence: Synthetic peptide
GGGGS

SEQ ID NO: 47 - protein, 10 aa, synthetic construct
  source 1..10: note = Description of Artificial Sequence: Synthetic peptide
GGGGSGGGGS

SEQ ID NO: 48 - protein, 15 aa, synthetic construct
  source 1..15: note = Description of Artificial Sequence: Synthetic peptide
GGGGSGGGGSGGGGS

SEQ ID NO: 49 - protein, 20 aa, synthetic construct
  source 1..20: note = Description of Artificial Sequence: Synthetic peptide
GGGGSGGGGSGGGGSGGGGS

SEQ ID NO: 50 - RNA, 15186 nt, Newcastle disease virus
  source 1..15186: mol_type = genomic RNA
accaaacaaagatttggtgaatgacgagactacactcaagaataattgtgcgcaacctttttttaagacatttatttgagttcgaattcgagtcctaaggagtcagggttcaacttttttctaacataatatgattaaatcaacaagaatacaattggccaaaaaaggagcctattaatatgtgattttcgttaagagtcacagttactgtaatatcctttgactgcattgcctatagttttcatgtagaatttttgttgagcacgagtcaagtacagtacagaagtgtctgtaaacatttctttgagtttggtaatacctaggacagccttcaaatatttagggcaggtactatgcttcaagtatgtccttagtgggataaggtcctccatggagatcatgcctttaagcactaggctgattatatcgcctattttattgagattttcgactctaagacctagtattgtaacctctaggatctgtctcgtgcactgtttaagtgatgtcctctttttcccgtcagtagagagattgtaaggggtaaatagaaatactgtgtcagcgagatcaccctcagcttccatatatatcacagaccggataactgtgtcaatgtgactagcgatgatctgggttatctgagttatgttcgctagcgtgctcaccagactctccgcacagaatggacggacgggctgtcccccggcttcaatcagcgcagtgtcaatattcttcctcaagtactttattaatcttggtaaaggactggataggatgtctgtcacacgctgccgctgtgaggtgaataacctggtcagtgtgatctcatctgatttagacaaaagcgtaccgtgccgctgcaccagagttttcgccatcctcaccacctcatgtacaaatgtaggcccgcccaggtaacccatgacaaataccaggtaacactccatatctcctcgacatgcataaccattagagagaatatatccttttgtggaacacggagcaaacaagttcatgagtagatgaaagtagtatcccattgcatacaacactttgatgattactaccccgccctcccttacagaatgcatggcaatcagagataaattgatagctagttgatctagtaagctttgattggaccctggaggaatttcaatgtcacaatgcagcaatgatacagatctgtagggcactgcagatgtaatataccccactactttatctgaggtcaggtcgctttcctctgtattttctctccataatggacggaactcttggacaaatccatctttgcatgttacctccgcctgtagattcctataaacaaccgaattcaaaaactgagttggggtcggcccgaaatgtcgttgcggggggttcatctcatttgaaaagagcgtattgtaatagatagtttcatgtggtacatgcagttcgagaagactcatgatggctccgctcccttcagctaagtataaggagttcccgtgtcttgcacatcttacctcgggtacagaaaggagatgagatgccttataccaagaggaagatgcagtccctatccctctgaacaagtatcgtactaagtagtcttcctccggatttggagatgtgagttcaggatgaatctgactaagtgtgaatgcgtcataccttgctggagcactcaaatctaactcttgcaaaaatgccgcaggttgtcgggtgaatggatctttcactcgagcaccaatatcttgcacagaagggaactctaataatggctcttgggggaacaacaacgccaggatagtatccctgtcctccctttccctgatcaaattgaggctcttccgagacatgtagtacagattagctgggaatgtaattatgttaggagacatgatagagctcacttgatcggggctaagtaatggtttcacagcatccgacagtaaatactcagtgagtattgaacatttctcttctgcagttaagcctcttattttcgggatttctcttgttgtagcaaagagtaccgtgtacagacagcataaccgggatatcagctgaagcatcttctcattcaggttatcatctaagacagatgggaacagcagatcatacttatttcctgaatataagccggagatcacacgtcgggtgcaagatactaatagttttgcagacatttcgataaaatccgtatctgcaagttggtgtgatccgtcatggttgaccaggcccactgcatgtaaccttgaatgaatgacgggatgagatattgtagctgcaatgttggaaagtagaattcctggcatattcttgtataaatcacccatatataagacaatattgtccaggcctcttactctcaggtaatagagttggtaagaacagtcgaggagcacttcaagtgctgcatattcaaataggcggaccacatctgaattctgagcttcactgatccaatttcgggtattgtcatacactattatggcgtcattctttatggaggtatcttcatcataagaaaccacagactggccaatcaacttcccgctggatattgaaagaatgttcattagctctatcgtgggatatgactccagattaagctcataactcttgaagatagctaagtcaagtctcgcaaagtctccctccgatacagggctaggatcatacataaacttatttgaggtcactgtcctcagttccggtaccaccccaagtagctcgaaaggaaccgcaacaggtgcttctctgatacagcaactaaatttactatgtaggtgcagtgtgatctcatcatatgtcctggttgttgtcattggaaagatcgattcgattagagataaacccaagagcatgatctgttggtaaaccacattcccctctttgactccttcttcagtgaacagcctttgagaatcattggatatgtgaatgtaaggtgacaccctgtagagagatgcaggggtgaatgtcatctgagttataccatcatctagtctatgttgaagattcccagccgtgggtaaaggggacagtaaccgaagatactctaagtttacattacaccgagattttgcaatcgtaagagcagcagtccaatttacttcattatccccataagcccagatcaacacggatgatgcccttagggcagcctttacatgtggcgacatatgagctatttttgcaagtgaggcagctctcctctcctgtgtctttgacccgagatatggtaccctcatcggaggattcttgctggtgtcatcggtcaattctatattgcttggaagatggaaccaagtaaattgttcatctccgctgtcacatcttgtacaccctccgcttacactaagaatctcaccctctacgagttctatcgtatcaggattagatacacccagtattttcctgcctcccgtcaaaggtgaccagcttctattccgtgcatagtctgccagtgtcagagaacacatattagaagagactaaggggtggttggatctactggaggaaaaaacatcgtctctaaacagcattgcatgcatgctagaataattgactatccgcatcagcctcttgatgcctaatggcctcctagtaagcgcaatcttaattacggtgtttgttgtgtcaacaagcccttgaatttgctttctcctacctacagagcttgcctccatgatggcatgcgcaacgcggggatgaatcacctcttgattaagcaagaattcagccaatgccttctcttctgcctcattatcctctgtgtgcactccagacaataagggatttgaacaagtttcaaataggactctttgcgtatgtttcttaagaacaatatttgggcttgcaacagtctcaaaattgaaagagtatgggtcgttgcacagactggcccaatctccattcccaggcggcctagttaagatattagtcataatgttaggactcagtaatcccactgcttctagtcgcttgatctctgcaaaagcagtagtccccgggtcaccgatatttctagtgtagagccttgagtattgaaggttactcagtccccctaattgggcaggagtcagaacatatgagtgcacaaaagagatgtcctcaatccacgactgattaagatcggggtgcgaattgttggtgatggagaactcagagtcaaagtatgtctgcacacaactcattatatagtttaaatagtaacagaagtctttgggaagcccgttctcgcatagccgtgctacagtagaggcaatgttggcacaggacattacggtgttttcactgagatcacctgacactagcactaatttagatgaatttttgaggacttgactgaggattgctccatctttgaagattcgtttgctgtatatgaagaatgtgtctgacctgatggtttcacgatccttcaaattatggccaatcaaatgattgacatgaattaattccttgaagaaattatcactggcttgatgcaactgtgtcaacaccatctccggagagtcgtctgatcttacctctctcgttactgctattacttgattatcaccctgtaccatacaggcaacacgacaatgcgatctagctgcagcaagttggattgcagcaattgagatcattgtccatagcttctggcataatccttcgataccccctctggcactgacaatatatatgtcatcattagggactcttgagaggtcacagtcagtagggtcacttggaggattgaaagggtctcctacgaacatcgtagtgtccatcagtcttaggtgaatccattcgaagaagtgaggtaggcccatcaactgattgatggcatgagcgaacaatttgattgtctgatatctccaattaagacagtacttttgcaggtcagttgttatgaaggttgcaactctccgacggttcttgcttttcggatcatgattgcggtttgaagatactctttctttacagtcagtgatacgtttcttattgctgttaaaagacagttgactcatcgctagcatactcttggtcaaggatatgctatcctgaatgactccatttccctgaaagaaaggtgcaatctgatcggctaggatcccttccgccatcacctgacagttccttaacttctttgtcagcttagcgaagatccgtccattaactttcacttccttctccttgagcgagtatgatactgccacattgtcatctctaaggtactcaagggtcgtcagatattccatctctttatatggatcaaaatcatttgactctaaaaactctatcaagaggcgattagtcgaagttgcttcttttacatgtttcttctggtcttcggagagaaggttccgcctaaacgaggcaagccaattatcgttggggtgtgcgattgccttgtcttttaggaacatgctcaggttggtgacagggtcatattctatacatggctcaaattcaagtgcagataaactcttatactctctcaacatgatatcgtgtgaaatctctgctgaatctgcatgtagttgcccaatgaccttcccatatattgtatccactttgactcgcggccacacacctgcattcttctttctgtacccgttgatgattgttcccttgaagaaagacagtacctgaaggatcatatcaaagtctaccattttcggtgcgcacatttggctcctgactgcctttgctgcaatacgggactcaagcagtgggtgaccccacagacgcaacagacacaacatctcagctgcttgattctgttctaaaccagagaatacagtagcgattgcatgagtcacggattctgctatatcattggggaggaggccaattagaatgtctttaagctcctgcaggttgaatgcgaagaaatctcctgcaaatgtacctgacggctcgagtagctggacagctccgtatgcaaatccctccattagtgatacaacatcgtagacttgattacccaagtcttttgccagagcgtctattaaccgcaaaatgtcatcaattttctctgataagcttctgagatgcaccgccgtggttgatattatgttgaccatatctctgccctccatcatatctgcatacatcaatacaagttcctgggtaagacatgtgaacttgttctcattcgtatgcgtcacaacgacaagttcaggagtgacaaagacttggcctaccttatgggttagcatcaccaatttgttggccgcagaccttgtcctagctgccaccatcagatgcctctggatctgttttatatggagccatgcaaacttggctgtggaccattttgagtgaaaccagaatgccggatccgtacgaatgctgctgaactcctctgaccgggggacattgttagaccaagatgaccccagcagtttcttctctatatgcgtacacagccttgtgaacagttctccatatctcgtgttgtgaatttggatcttcttctcaatcttccgaaatttgttggttgaatctgggacctcaatattagccagttcttctaaacacctggggtggagcactccggttattctggaattgtggttaagagtttggtgtactgcccttccgagttttatcattctctcagtatcaggagaggccgattcaagtatttttttccattgtcggctgagaatgaggtggtcgaagtcacattcatcaggaagcggtagcccagttaatttccagtaatagagtagtttgtgcttgaccaatggtgaagacaggtgtgactctggtaggataatctgatgctctgccctttcaggaccggagctcgccatgtcctacccgtattgttaaccatgagctgttttgccttgtatctcattgccacttacattttttcttaatcaagagactattgacaaggcttaatctgatcgcatgagcactggctgattgtggtcaactggcaacatggaattcgagcacgcgccggcattcggtttgattcttgatgtcgcagaagataggtgatacaatgccatctttccaactcctttataattgactcaactagccagacctggcttctctaaccccgtcatctttgaggatctcaactagtaacgggacgattctgaattctccgaagagagtattagatatttcagcaatgctgagacaataggtcttattagtcttgaccactttaaaacaagttgatgttgtgtatgctgctttggtactgcttgaactcactcgagtaatgcgactgcgggatgtgctatcgaatactgcagacgcagggttaagtcttgcttgtacaccatcaagcattgtcccgaatacccctcgcaaggtgtggtttctatagaagattaggggatatggatctgtatagactccagtaacacacgagttggggcatcttgctgaagcctggcaagggatactacctggccgagtgaaggcattgaatgtataaggactatgaagagtggctgttttgttgctgactgtcataggatataataacgcgggagagaagtatgatgaccctcgttgatacaagaaatgagatgtccctactgtgagaattctgccttcggcccccatgagtgtgactgtgttgggcggtacagtcagtaccgggtcttcgcctaaggatgttgacaccttgatagataagatagcctgctgtatgcgtttcccaccaaaccgtccaggcttatacgaagacttggccattcgaatctggtagtcttgctcatctgggcatgtgtcattgtatcgcttgtatatcacatatttcccttcctgtacagtgtcactgggtgaattgggttttaaccctccgtagactgagaaccatacgcggctgtcaataaaagatccaccccctactcctgggtagttggccacccagtccccgaataatgttgtgacatctaggtccttttcgtggtactggccgtcgaaccctaacctcccatgtaccatccgcgtagggacagctgagttataatcttcttcctctgtctccgtgactttcgagcacagcatatcacaacccaggggagttgcactcacactgcaagacttccgattttgggtgtcgtccaggttgatggaacgcagagtagaaaagaataccctccctgttgcagatgtccggagcacaccaagtgctaaatactgatatgaatgtgagtgatctctgcatccagacaatattacattatgggtgtagcagtaatgggtagcactcatgtcaaatgagggtattcgagtgcaacctgatcctgtagtaggcgccgggataaaattcagatgttcttgaaatgcagagggatagaatgatgtgacatcactagcatcatctacaatgagttctttgcctatcccccctatataatctgggtcatggataggtgccccccacccactgttgtttgcagctccattaatctgataagagagagatgttattgcgttcataattgtggtctcagtatttaacaatgccaacggagactcaagggccacttgcttatatatcctatctactacatcttgattggaaccaagtgtagatgtaatcttttcttctgccctggaaatcctagtcggtatgcctacaagatcgctaggtgtgctagcccccatgctatataaaagggaggctacagatatagccaaggtcactactgttaagaataagattgcaatccggaatatcaagcgccatgtattttttgcctctctttcatcattctctaacgcaacttggctaacggcgcggtccatgattgaggactgttgtcggtgaagcggtagaacggaggttgtgaagcctggctcgcaattgaggggcggcctctcttaccgttctacccgtatatcgtcctttggtcatctacaaccggtagttttttcttaactctctgaactgacagactaccagaactttcacacaaattactattagggaaaccttcgttcctcatctgtgttcacatttttgtagtggctctcatctgatctagagtattattcccaagccataataaggtcttttgttgcgccttttgcttgtacattaggtagcatgctagaatcaggctaagtataccaaaaacaagagatatgatagtcaaaacgatataggtaatgagagcagatgtgctagtcagtttgacattgactttgtctagttttctgttgctttcctctaacttattcaaagcattactgatcgagttgttgacattcccaagctcagttgagatatcaagattgcctgttattattacttgagaatcttgtattgagatattcttctgataagttacatcgaattccccactgagccttaaagttatcccgcctaaggataaaacattgcatgattgtttatctattagagacacggcttctccatagttttgcgatatgatacccggggggtttacacatctacatgttgtcatcttgcagttggcgatgactgaacctttgatagtcatgtatggtgtagtaagtgcgccttcggtctttgagtacatacaggccgacgtattgccgctcaagcaggaataaataccaggggacatagggaacgttactattcttgtacaatataaatctaagtcagtttctatacagtatgaggtgtcaagttcttctatcacagaaccgacctgtgtcaccacttttgggacaagtgccgaggcaaatcccctggttgtgcttacggataaggtttccaagtaggtggcacgcatattatttaggttcccgactgaaggtgcagttacctgtatacccaagagttgagtctgtgagtcgtatagaatagggttaccggtgattaagccgctaccgattaatgagctgagttgattgttccctacacctaacttagtcaataagtaatccatatttccaccagctagattgtaaagtgcctgaatagtcagcttgtttaaagcaggtgaagtgatttgtggtccgaatactgtagtcaattcggttaggtacaggttgagctctacaccaacttgctgtgcaattttgatgcagtctaattcctgagctgttttattaaattggtcattaacaaactgctgcatcttcccaactgccactgctagttgcgataatccgtcagtgacctcatgcacagcctcattggttgcggcaatgctctctttaagtcggaggatgttggcagcattttgtttggcttgtatcagagctgcggccgctgttatttgtgcggcagttgcaaccccaagagccacaccgccaataatggcgcctataaggcgcccctgtctcccccctccagatgtagtcacagactcttgtatcctacggatagagtcaccaaggggggtgagcaaagtggtcaatgtcctgttgtatgcatccaagggggctttcgcacatgcctccttatccttgggcagattcgggaggagcttaactatgattgatcctgtctgggatgaggtgtatatgttgacggctttgtctcctgtaaccacaattcctgcagctgcaagaggcctgccatcaatggagtttgccggacagatgcaactcagtaccagcgcaacccggatagtcagcatcataggtgctgggttcttggtagaaggtctggagcccatcttgcacctggagggcgccaaccgggatccagaatcttctacccgtgttttttctaacttgatagacaggtaaactaactgtaaataatcaagacagattagttttttgtgtcatgatgatctgggtgagtgggcggagcgcaatctcagagacgcagcttatttcttaaaaggattgtatttggcaagggtgtgccccttctccagcttagtagaggtaacctcgtggtcggcggtcactgcgacagcgcgttgggtacctgcttggataatgattttaacgctccgcaggcacgcggtttgactccagagtatcttggccacctgaggagaagcatttgctatgggatagcaggctgtcccactgctagagaagaaaggtgccaaaagcttagtccgtgcacctcttgcttttaccaacacggaaggcccgagcacatcactgagcccgacagatagatcaaggctccttattttcttttccagcttgtcaaatgtcactttcttccccttcctatctacagtggtcataagtccaatatgcaagaagaggttagcatagtatccgctgtcagacttagacagagatttaaccaaaggactcctcgggtctacctccacattaatagtgacattgagcgcaagattgtacagactcgagccagaaaccttcaatactgcagctgggatcttgtagacatccctcttcggtaccacagtcaaggagacaaagttcaccttgtattctagggttccactcccgggaatcttctctggcgctttcacgtgcttgactgcattcactgatgagtatttgtttgccacaaccctacagctttgcagcacttggggtgcctgcactactgagaaaaccattctctcagtattagttgcactcttcttgcatgtgactatcatagtgagacaggcccttgccagctcaataaggtctccggtatttgggacgcttcctaggcagagcatcgcagcggaaagtaactcgcgcttgggtttatcatcgatcatgccgacggtggcttcttcattcccaacttgaaagatgaatccataggtggtgatgaatactgagtcctccttactatcagtccacaagtcaaggcgctggatcctatattgcggggcgatttgcttcttcccatctcctgtgtcttgtaggacgatcggaaatgctaacaggttgctagaagaatgggcagaatcaaagtacagcccaattgtcctagatgagtccatcttggcacaattggggcactccaattctacccgtattttttcttaatcttaaatgtagctagattaattacggttacgcgatcattcagtggggctgaggaagcgagagaggattgccgcttggagagttggaccttgggtccgcgggggggaactcggtgcagattccgtgtgatgccgagtggacagggacccgctacgtgtggcagtagtaattagccatttagagcaaggcgcttgattttcctgatttcctcgatcgacccggctgcatctaacttgcttaggagcttggctgaagaactcgggtgcattgggcgtgacatgatcaatgcacggacagtgtccttttccactcctatatcaggcccgcatgcagtggcgggtttaatcaattcagatggatgtggcactggttgcgaaagtttattaagtgccatttcgcctccttgtgtcacatagggagaggggtctccagggcctgaaactaaaaccgggtgagatcgggcaactgcccgtagatcactcagagatgaaatgttggcacaaccgggatccagaatcttcatcattcccaagttggcttccatgactgcaacagatgttttcagctgttggatttcggaccgcatcatagggatggaggatgtctgtttcaagacaagatctagctgatagtcaaccttacttactctctgtgatatcgcctccatcatagacatcatcgcttgcacaaagtctacaggtggctggacatgatccgcagatacaagggtattgtcttggctctgccttgatcggagagcatgaggggttgcaccagctgatagttgtgactcctcccattgtccatgatatgctgtgttcacgtctgtgccctggtttccaggggcggccttgacttggttctgcggtctttcctgactgtttccgcgactgggttgactcccctgctgttgagtcggacgttggtgattcccctcttgggggctcgaccatgggccctttttagcattggacgatttattgctgagcttgtcaagcatcaacagcagagagttgcttgctccggtcctgagctgtgtgtcgacggcttcgtctgtggcctgggtggggggctggtcggcggatgtggccggcgggctgtcatgcggggtcgtttgctcgggtgtggatggttgtttgtcagatctgtcctgtcgatcggggttgtcttgactggccggtggctggatgctcccatgcttctcccatgctgcgctcagcaccttggtcttgccttgtgggattgcactccttccaacagtctctgctggtttaccctgggctgtaattatgttgtcaatgacagttccacttgtctcaaatagctcgtcgatctctgcatctgtaaaggtggccatgtttgtcctggtctatcaggtagaggagagagcagagactcgggagacttgccctgatctctgaatatccctcttctacccgtacttttttctaattccctcggctctgttttgattgttagtgagccgcattgtgcctgtggtatctagggcgtacgtagttgtacagcagggggagggaggaaagagggggatgtttgtttgagggtgtggtcatatagagccgcaaatcgaggggtcgactacgggtgagggcattgggatgtttttgtggaagcaggctgggttttgtccatcaatacccccagtcggtgtcgttatcttgggatggcccaggagttgggggaggccccgattggggagtgccctgtgcagagtttggcgcctccctcatgctatttgctaccgctctcatcagatccaggaattgggtctccccatccccggcttctggttgcccttgcgatctattcgatccgccttgtagagcttgggaccccccctcgctaagcccagtgaggactccgacttgttgagtaggcatgtctatgctgctggtctcctcggagacccgttgggcagcagctgccaggcccctccttgctgctggggttagctttagctcggcagccatatcctcgttaatgctacttccctgagcctgagcgtactctactccaagtctccagaatgatgtgctcataaagtccctggcaaattggtatttcccagtacctttatctaggactgatgccatacccatggcaaaggagtaaagttgtgcatactcggcaggcgcaaagctcatctggtcactatcaccaagtaatgtcatgtacggcgcattatctcctttcatccgatacaaacgcatgagctgcttcatcttctggatgtcgcctgagaggctactaagtgcaagggctgatgtcttggtgttgattccgtacttgagtgtcaagaagaatgcagtaagcccggtattcctgatgtatgagtctacgtcccctaccaggttataataagtagaggtaccacctgccgtgttgcggcctctcttgagctcgctaaccaaaaagatgcggactgcaagagactgtctgatcgtgagttggattgtgctcctgcatacggggtagaggatgtatttcttttggaccctgccttgctgcatatacttattgattcgccttgtttccgactcatctgcagtctcatacgcagtcatggcttttgctactgtgacccatacttgagcctggatagagaggatcctctccagggtatcggtgatgtcttctggtgcatcatcttcggccccggctgtgacgaacggggttccgttgctgcatgcccgagggagagatcctgctatcatcgcaaatctctgtgctctctcttcagacactccactcctattgttgaactggggcgtgccgttggcaaagccatcaatctcaagcacggccaatgtggcttcattctgtttccctgcaagggcaacatggttcctcattacctgtgagtgggagcataaaagagatatgagagcaccttgcctgagtggtttgttggcatcttcgctaacagcaatccggaggcagaataccacaaagctccatctatcttctgggtcatcactgttaagagtgaataccgggacgtctacttttaaggtactccctttttctccccctccatgagctccattggggcgagtctgagccgcgaggagctgttcgtactcatcaaatacggaagacatgttggcagaaggctttctcgagtttgtgcttcgggctcgcactcgagattcacaccttctacccgtgcgacttcaattgctccttcgccttttatcgtaactcacggattctctgtttggt

SEQ ID NO: 51 - RNA, 16824 nt, synthetic construct
  source 1..16824: mol_type = other RNA; note = Description of Artificial Sequence: Synthetic polynucleotide
accaaacaaagatttggtgaatgacgagactacactcaagaataattgtgcgcaacctttttttaagacatttatttgagttcgaattcgagtcctaaggagtcagggttcaacttttttctaacataatatgattaaatcaacaagaatacaattggccaaaaaaggagcctattaatatgtgattttcgttaagagtcacagttactgtaatatcctttgactgcattgcctatagttttcatgtagaatttttgttgagcacgagtcaagtacagtacagaagtgtctgtaaacatttctttgagtttggtaatacctaggacagccttcaaatatttagggcaggtactatgcttcaagtatgtccttagtgggataaggtcctccatggagatcatgcctttaagcactaggctgattatatcgcctattttattgagattttcgactctaagacctagtattgtaacctctaggatctgtctcgtgcactgtttaagtgatgtcctttttttcccgtcagtagagagattgtaaggggtaaatagaaatactgtgtcagcgagatcaccctcagcttccatatatatcacagaccggataactgtgtcaatgtgactagcgatgatctgggttatctgagttatgttcgctagcgtgctcaccagactctccgcacagaatggacggacgggctgtcccccggcttcaatcagcgcagtgtcaatattcttcctcaagtactttattaatcttggtaaaggactggataggatgtctgtcacacgctgccgctgtgaggtgaataacctggtcagtgtgatctcatctgatttagacaaaagcgtaccgtgccgctgcaccagagttttcgccatcctcaccacctcatgtacaaatgtaggcccgcccaggtaacccatgacaaataccaggtaacactccatatctcctcgacatgcataaccattagagagaatatatccttttgtggaacacggagcaaacaagttcatgagtagatgaaagtagtatcccattgcatacaacactttgatgattactaccccgccctcccttacagaatgcatggcaatcagagataaattgatagctagttgatctagtaagctttgattggaccctggaggaatttcaatgtcacaatgcagcaatgatacagatctgtagggcactgcagatgtaatataccccactactttatctgaggtcaggtcgctttcctctgtattttctctccataatggacggaactcttggacaaatccatctttgcatgttacctccgcctgtagattcctataaacaaccgaattcaaaaactgagttggggtcggcccgaaatgtcgttgcggggggttcatctcatttgaaaagagcgtattgtaatagatagtttcatgtggtacatgcagttcgagaagactcatgatggctccgctcccttcagctaagtataaggagttcccgtgtcttgcacatcttacctcgggtacagaaaggagatgagatgccttataccaagaggaagatgcagtccctatccctctgaacaagtatcgtactaagtagtcttcctccggatttggagatgtgagttcaggatgaatctgactaagtgtgaatgcgtcataccttgctggagcactcaaatctaactcttgcaaaaatgccgcaggttgtcgggtgaatggatctttcactcgagcaccaatatcttgcacagaagggaactctaataatggctcttgggggaacaacaacgccaggatagtatccctgtcctccctttccctgatcaaattgaggctcttccgagacatgtagtacagattagctgggaatgtaattatgttaggagacatgatagagctcacttgatcggggctaagtaatggtttcacagcatccgacagtaaatactcagtgagtattgaacatttctcttctgcagttaagcctcttattttcgggatttctcttgttgtagcaaagagtaccgtgtacagacagcataaccgggatatcagctgaagcatcttctcattcaggttatcatctaagacagatgggaacagcagatcatacttatttcctgaatataagccggagatcacacgtcgggtgcaagatactaatagttttgcagacatttcgataaaatccgtatctgcaagttggtgtgatccgtcatggttgaccaggcccactgcatgtaaccttgaatgaatgacgggatgagatattgtagctgcaatgttggaaagtagaattcctggcatattcttgtataaatcacccatatataagacaatattgtccaggcctcttactctcaggtaatagagttggtaagaacagtcgaggagcacttcaagtgctgcatattcaaataggcggaccacatctgaattctgagcttcactgatccaatttcgggtattgtcatacactattatggcgtcattctttatggaggtatcttcatcataagaaaccacagactggccaatcaacttcccgctggatattgaaagaatgttcattagctctatcgtgggatatgactccagattaagctcataactcttgaagatagctaagtcaagtctcgcaaagtctccctccgatacagggctaggatcatacataaacttatttgaggtcactgtcctcagttccggtaccaccccaagtagctcgaaaggaaccgcaacaggtgcttctctgatacagcaactaaatttactatgtaggtgcagtgtgatctcatcatatgtcctggttgttgtcattggaaagatcgattcgattagagataaacccaagagcatgatctgttggtaaaccacattcccctctttgactccttcttcagtgaacagcctttgagaatcattggatatgtgaatgtaaggtgacaccctgtagagagatgcaggggtgaatgtcatctgagttataccatcatctagtctatgttgaagattcccagccgtgggtaaaggggacagtaaccgaagatactctaagtttacattacaccgagattttgcaatcgtaagagcagcagtccaatttacttcattatccccataagcccagatcaacacggatgatgcccttagggcagcctttacatgtggcgacatatgagctatttttgcaagtgaggcagctctcctctcctgtgtctttgacccgagatatggtaccctcatcggaggattcttgctggtgtcatcggtcaattctatattgcttggaagatggaaccaagtaaattgttcatctccgctgtcacatcttgtacaccctccgcttacactaagaatctcaccctctacgagttctatcgtatcaggattagatacacccagtattttcctgcctcccgtcaaaggtgaccagcttctattccgtgcatagtctgccagtgtcagagaacacatattagaagagactaaggggtggttggatctactggaggaaaaaacatcgtctctaaacagcattgcatgcatgctagaataattgactatccgcatcagcctcttgatgcctaatggcctcctagtaagcgcaatcttaattacggtgtttgttgtgtcaacaagcccttgaatttgctttctcctacctacagagcttgcctccatgatggcatgcgcaacgcggggatgaatcacctcttgattaagcaagaattcagccaatgccttctcttctgcctcattatcctctgtgtgcactccagacaataagggatttgaacaagtttcaaataggactctttgcgtatgtttcttaagaacaatatttgggcttgcaacagtctcaaaattgaaagagtatgggtcgttgcacagactggcccaatctccattcccaggcggcctagttaagatattagtcataatgttaggactcagtaatcccactgcttctagtcgcttgatctctgcaaaagcagtagtccccgggtcaccgatatttctagtgtagagccttgagtattgaaggttactcagtccccctaattgggcaggagtcagaacatatgagtgcacaaaagagatgtcctcaatccacgactgattaagatcggggtgcgaattgttggtgatggagaactcagagtcaaagtatgtctgcacacaactcattatatagtttaaatagtaacagaagtctttgggaagcccgttctcgcatagccgtgctacagtagaggcaatgttggcacaggacattacggtgttttcactgagatcacctgacactagcactaatttagatgaatttttgaggacttgactgaggattgctccatctttgaagattcgtttgctgtatatgaagaatgtgtctgacctgatggtttcacgatccttcaaattatggccaatcaaatgattgacatgaattaattccttgaagaaattatcactggcttgatgcaactgtgtcaacaccatctccggagagtcgtctgatcttacctctctcgttactgctattacttgattatcaccctgtaccatacaggcaacacgacaatgcgatctagctgcagcaagttggattgcagcaattgagatcattgtccatagcttctggcataatccttcgataccccctctggcactgacaatatatatgtcatcattagggactcttgagaggtcacagtcagtagggtcacttggaggattgaaagggtctcctacgaacatcgtagtgtccatcagtcttaggtgaatccattcgaagaagtgaggtaggcccatcaactgattgatggcatgagcgaacaatttgattgtctgatatctccaattaagacagtacttttgcaggtcagttgttatgaaggttgcaactctccgacggttcttgcttttcggatcatgattgcggtttgaagatactctttctttacagtcagtgatacgtttcttattgctgttaaaagacagttgactcatcgctagcatactcttggtcaaggatatgctatcctgaatgactccatttccctgaaagaaaggtgcaatctgatcggctaggatcccttccgccatcacctgacagttccttaacttctttgtcagcttagcgaagatccgtccattaactttcacttccttctccttgagcgagtatgatactgccacattgtcatctctaaggtactcaagggtcgtcagatattccatctctttatatggatcaaaatcatttgactctaaaaactctatcaagaggcgattagtcgaagttgcttcttttacatgtttcttctggtcttcggagagaaggttccgcctaaacgaggcaagccaattatcgttggggtgtgcgattgccttgtcttttaggaacatgctcaggttggtgacagggtcatattctatacatggctcaaattcaagtgcagataaactcttatactctctcaacatgatatcgtgtgaaatctctgctgaatctgcatgtagttgcccaatgaccttcccatatattgtatccactttgactcgcggccacacacctgcattcttctttctgtacccgttgatgattgttcccttgaagaaagacagtacctgaaggatcatatcaaagtctaccattttcggtgcgcacatttggctcctgactgcctttgctgcaatacgggactcaagcagtgggtgaccccacagacgcaacagacacaacatctcagctgcttgattctgttctaaaccagagaatacagtagcgattgcatgagtcacggattctgctatatcattggggaggaggccaattagaatgtctttaagctcctgcaggttgaatgcgaagaaatctcctgcaaatgtacctgacggctcgagtagctggacagctccgtatgcaaatccctccattagtgatacaacatcgtagacttgattacccaagtcttttgccagagcgtctattaaccgcaaaatgtcatcaattttctctgataagcttctgagatgcaccgccgtggttgatattatgttgaccatatctctgccctccatcatatctgcatacatcaatacaagttcctgggtaagacatgtgaacttgttctcattcgtatgcgtcacaacgacaagttcaggagtgacaaagacttggcctaccttatgggttagcatcaccaatttgttggccgcagaccttgtcctagctgccaccatcagatgcctctggatctgttttatatggagccatgcaaacttggctgtggaccattttgagtgaaaccagaatgccggatccgtacgaatgctgctgaactcctctgaccgggggacattgttagaccaagatgaccccagcagtttcttctctatatgcgtacacagccttgtgaacagttctccatatctcgtgttgtgaatttggatcttcttctcaatcttccgaaatttgttggttgaatctgggacctcaatattagccagttcttctaaacacctggggtggagcactccggttattctggaattgtggttaagagtttggtgtactgcccttccgagttttatcattctctcagtatcaggagaggccgattcaagtatttttttccattgtcggctgagaatgaggtggtcgaagtcacattcatcaggaagcggtagcccagttaatttccagtaatagagtagtttgtgcttgaccaatggtgaagacaggtgtgactctggtaggataatctgatgctctgccctttcaggaccggagctcgccatgtcctacccgtattgttaaccatgagctgttttgccttgtatctcattgccacttacattttttcttaatcaagagactattgacaaggcttgatctgatcgcatgagcactggctgattgtggtcaactggcaacatggaattcgagcacgcgccggcattcggtttgattcttgatgtcgcagaagataggtgatacaatgccatctttccaactcctttataattgactcaactagccagacctggcttctctaaccccgtcatctttgaggatctcaactagtaacgggacgattctgaattctccgaagagagtattagatatttcagcaatgctgagacaataggtcttattagtcttgaccactttaaaacaagttgatgttgtgtatgctgctttggtactgcttgaactcactcgagtaatgcgactgcgggatgtgctatcgaatactgcagacgcagggttaagtcttgcttgtacaccatcaagcattgtcccgaatacccctcgcaaggtgtggtttctatagaagattaggggatatggatctgtatagactccagtaacacacgagttggggcatcttgctgaagcctggcaagggatactacctggccgagtgaaggcattgaatgtataaggactatgaagagtggctgttttgttgctgactgtcataggatataataacgcgggagagaagtatgatgaccctcgttgatacaagaaatgagatgtccctactgtgagaattctgccttcggcccccatgagtgtgactgtgttgggcggtacagtcagtaccgggtcttcgcctaaggatgttgacaccttgatagataagatagcctgctgtatgcgtttcccaccaaaccgtccaggcttatacgaagacttggccattcgaatctggtagtcttgctcatctgggcatgtgtcattgtatcgcttgtatatcacatatttcccttcctgtacagtgtcactgggtgaattgggttttaaccctccgtagactgagaaccatacgcggctgtcaataaaagatccaccccctactcctgggtagttggccacccagtccccgaataatgttgtgacatctaggtccttttcgtggtactggccgtcgaaccctaacctcccatgtaccatccgcgtagggacagctgagttataatcttcttcctctgtctccgtgactttcgagcacagcatatcacaacccaggggagttgcactcacactgcaagacttccgattttgggtgtcgtccaggttgatggaacgcagagtagaaaagaataccctccctgttgcagatgtccggagcacaccaagtgctaaatactgatatgaatgtgagtgatctctgcatccagacaatattacattatgggtgtagcagtaatgggtagcactcatgtcaaatgagggtattcgagtgcaacctgatcctgtagtaggcgccgggataaaattcagatgttcttgaaatgcagagggatagaatgatgtgacatcactagcatcatctacaatgagttctttgcctatcccccctatataatctgggtcatggataggtgccccccacccactgttgtttgcagctccattaatctgataagagagagatgttattgcgttcataattgtggtctcagtatttaacaatgccaacggagactcaagggccacttgcttatatatcctatctactacatcttgattggaaccaagtgtagatgtaatcttttcttctgccctggaaatcctagtcggtatgcctacaagatcgctaggtgtgctagcccccatgctatataaaagggaggctacagatatagccaaggtcactactgttaagaataagattgcaatccggaatatcaagcgccatgtattttttgcctctctttcatcattctctaacgcaacttggctaacggcgcggtccatgattgaggactgttgtcggtgaagcggtagaacggaggttgtgaagcctggctcgcaattgaggggcggcctctcttaccgttctacccgtatatcgtcctttggtcatctacaaccggtagttttttcttaactctctgaactgacagactaccagaactttcacacaaattactattagggaaaccttcgttcctcatctgtgttcacatttttgtagtggctctcatctgatctagagtattattcccaagccataataaggtcttttgttgcgccttttgcttgtacattaggtagcatgctagaatcaggctaagtataccaaaaacaagagatatgatagtcaaaacgatataggtaatgagagcagatgtgctagtcagtttgacattgactttgtctagttttctgttgctttcctctaacttattcaaagcattactgatcgagttgttgacattcccaagctcagttgagatatcaagattgcctgttattattacttgagaatcttgtattgagatattcttctgataagttacatcgaattccccactgagccttaaagttatcccgcctaaggataaaacattgcatgattgtttatctattagagacacggcttctccatagttttgcgatatgatacccggggggtttacacatctacatgttgtcatcttgcagttggcgatgactgaacctttgatagtcatgtatggtgtagtaagtgcgccttcggtctttgagtacatacaggccgacgtattgccgctcaagcaggaataaataccaggggacatagggaacgttactattcttgtacaatataaatctaagtcagtttctatacagtatgaggtgtcaagttcttctatcacagaaccgacctgtgtcaccacttttgggacaagtgccgaggcaaatcccctggttgtgcttacggataaggtttccaagtaggtggcacgcatattatttaggttcccgactgaaggggcagttacctgtatacccaagagttgagtctgtgagtcgtatagaatagggttaccggtgattaagccgctaccgattaatgagctgagttgattgttccctacacctaacttagtcaataagtaatccatatttccaccagctagattgtaaagtgcctgaatagtcagcttgtttaaagcaggtgaagtgatttgtggtccgaatactgtagtcaattcggttaggtacaggttgagctctacaccaacttgctgtgcaattttgatgcagtctaattcctgagctgttttattaaattggtcattaacaaactgctgcatcttcccaactgccactgctagttgcgataatccgtcagtgacctcatgcacagcctcattggttgcggcaatgctctctttaagtcggaggatgttggcagcattttgtttggcttgtatcagagctgcggccgctgttatttgtgcggcagttgcaaccccaagagccacaccgccaataatggcgcctataaggcgcccctgtctcccccctccagatgtagtcacagactcttgtatcctacggatagagtcaccaaggggggtgagcaaagtggtcaatgtcctgttgtatgcatccaagggggctttcgcacatgcctccttatccttgggcagattcgggaggagcttaactatgattgatcctgtctgggatgaggtgtatatgttgacggctttgtctcctgtaaccacaattcctgcagctgcaagaggcctgccatcaatggagtttgccggacagatgcaactcagtaccagcgcaacccggatagtcagcatcataggtgctgggttcttggtagaaggtctggagcccatcttgcacctggagggcgccaaccgggatccagaatcttctacccgtgttttttctaacttgatagacaggtaaactaactgtaaataatcaagacagattagttttttgtgtcatgatgatctgggtgagtgggcggagcgcaatctcagagacgcagcttatttcttaaaaggattgtatttggcaagggtgtgccccttctccagcttagtagaggtaacctcgtggtcggcggtcactgcgacagcgcgttgggtacctgcttggataatgattttaacgctccgcaggcacgcggtttgactccagagtatcttggccacctgaggagaagcatttgctatgggatagcaggctgtcccactgctagagaagaaaggtgccaaaagcttagtccgtgcacctcttgcttttaccaacacggaaggcccgagcacatcactgagcccgacagatagatcaaggctccttattttcttttccagcttgtcaaatgtcactttcttccccttcctatctacagtggtcataagtccaatatgcaagaagaggttagcatagtatccgctgtcagacttagacagagatttaaccaaaggactcctcgggtctacctccacattaatagtgacattgagcgcaagattgtacagactcgagccagaaaccttcaatactgcagctgggatcttgtagacatccctcttcggtaccacagtcaaggagacaaagttcaccttgtattctagggttccactcccgggaatcttctctggcgctttcacgtgcttgactgcattcactgatgagtatttgtttgccacaaccctacagctttgcagcacttggggtgcctgcactactgagaaaaccattctctcagtattagttgcactcttcttgcatgtgactatcatagtgagacaggcccttgccagctcaataaggtctccggtatttgggacgcttcctaggcagagcatcgcagcggaaagtaactcgcgcttgggtttatcatcgatcatgccgacggtggcttcttcattcccaacttgaaagatgaatccataggtggtgatgaatactgagtcctccttactatcagtccacaagtcaaggcgctggatcctatattgcggggcgatttgcttcttcccatctcctgtgtcttgtaggacgatcggaaatgctaacaggttgctagaagaatgggcagaatcaaagtacagcccaattgtcctagatgagtccatcttggcacaattggggcactccaattctacccgtattttttcttaatcttaaatgtagctagattaattacggttacgcgatcattcagtggggctgaggaagcgagagaggattgccgcttggagagttggaccttgggtccgcggatcattaggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcgagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctagatccgccgccaccgccaccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacccatggtggcggttctacccgtattttttctaaccgcgggggggaactcggtgcagattccgtgtgatgccgagtggacagggacccgctacgtgtggcagtagtaattagccatttagagcaaggcgcttgattttcctgatttcctcgatcgacccggctgcatctaacttgcttaggagcttggctgaagaactcgggtgcattgggcgtgacatgatcaatgcacggacagtgtccttttccactcctatatcaggcccgcatgcagtggcgggtttaatcaattcagatggatgtggcactggttgcgaaagtttattaagtgccatttcgcctccttgtgtcacatagggagaggggtctccagggcctgaaactaaaaccgggtgagatcgggcaactgcccgtagatcactcagagatgaaatgttggcacaaccgggatccagaatcttcatcattcccaagttggcttccatgactgcaacagatgttttcagctgttggatttcggaccgcatcatagggatggaggatgtctgtttcaagacaagatctagctgatagtcaaccttacttactctctgtgatatcgcctccatcatagacatcatcgcttgcacaaagtctacaggtggctggacatgatccgcagatacaagggtattgtcttggctctgccttgatcggagagcatgaggggttgcaccagctgatagttgtgactcctcccattgtccatgatatgctgtgttcacgtctgtgccctggtttccaggggcggccttgacttggttctgcggtctttcctgactgtttccgcgactgggttgactcccctgctgttgagtcggacgttggtgattcccctcttgggggctcgaccatgggccctttttagcattggacgatttattgctgagcttgtcaagcatcaacagcagagagttgcttgctccggtcctgagctgtgtgtcgacggcttcgtctgtggcctgggtggggggctggtcggcggatgtggccggcgggctgtcatgcggggtcgtttgctcgggtgtggatggttgtttgtcagatctgtcctgtcgatcggggttgtcttgactggccggtggctggatgctcccatgcttctcccatgctgcgctcagcaccttggtcttgccttgtgggattgcactccttccaacagtctctgctggtttaccctgggctgtaattatgttgtcaatgacagttccacttgtctcaaatagctcgtcgatctctgcatctgtaaaggtggccatgtttgtcctggtctatcaggtagaggagagagcagagactcgggagacttgccctgatctctgaatatccctcttctacccgtacttttttctaattccctcggctctgttttgattgttagtgagccgcattgtgcctgtggtatctagggcgtacgtagttgtacagcagggggagggaggaaagagggggatgtttgtttgagggtgtggtcatatagagccgcaaatcgaggggtcgactacgggtgagggcattgggatgtttttgtggaagcaggctgggttttgtccatcaatacccccagtcggtgtcgttatcttgggatggcccaggagttgggggaggccccgattggggagtgccctgtgcagagtttggcgcctccctcatgctatttgctaccgctctcatcagatccaggaattgggtctccccatccccggcttctggttgcccttgcgatctattcgatccgccttgtagagcttgggaccccccctcgctaagcccagtgaggactccgacttgttgagtaggcatgtctatgctgctggtctcctcggagacccgttgggcagcagctgccaggcccctccttgctgctggggttagctttagctcggcagccatatcctcgttaatgctacttccctgagcctgagcgtactctactccaagtctccagaatgatgtgctcataaagtccctggcaaattggtatttcccagtacctttatctaggactgatgccatacccatggcaaaggagtaaagttgtgcatactcggcaggcgcaaagctcatctggtcactatcaccaagtaatgtcatgtacggcgcattatctcctttcatccgatacaaacgcatgagctgcttcatcttctggatgtcgcctgagaggctactaagtgcaagggctgatgtcttggtgttgattccgtacttgagtgtcaagaagaatgcagtaagcccggtattcctgatgtatgagtctacgtcccctaccaggttataataagtagaggtaccacctgccgtgttgcggcctctcttgagctcgctaaccaaaaagatgcggactgcaagagactgtctgatcgtgagttggattgtgctcctgcatacggggtagaggatgtatttcttttggaccctgccttgctgcatatacttattgattcgccttgtttccgactcatctgcagtctcatacgcagtcatggcttttgctactgtgacccatacttgagcctggatagagaggatcctctccagggtatcggtgatgtcttctggtgcatcatcttcggccccggctgtgacgaacggggttccgttgctgcatgcccgagggagagatcctgctatcatcgcaaatctctgtgctctctcttcagacactccactcctattgttgaactggggcgtgccgttggcaaagccatcaatctcaagcacggccaatgtggcttcattctgtttccctgcaagggcaacatggttcctcattacctgtgagtgggagcataaaagagatatgagagcaccttgcctgagtggtttgttggcatcttcgctaacagcaatccggaggcagaataccacaaagctccatctatcttctgggtcatcactgttaagagtgaataccgggacgtctacttttaaggtactccctttttctccccctccatgagctccattggggcgagtctgagccgcgaggagctgttcgtactcatcaaatacggaagacatgttggcagaaggctttctcgagtttgtgcttcgggctcgcactcgagattcacaccttctacccgtgcgacttcaattgctccttcgccttttatcgtaactcacggattctctgtttggt

SEQ ID NO: 52 - RNA, 16824 nt, synthetic construct
  source 1..16824: mol_type = other RNA; note = Description of Artificial Sequence: Synthetic polynucleotide
accaaacaaagatttggtgaatgacgagactacactcaagaataattgtgcgcaacctttttttaagacatttatttgagttcgaattcgagtcctaaggagtcagggttcaacttttttctaacataatatgattaaatcaacaagaatacaattggccaaaaaaggagcctattaatatgtgattttcgttaagagtcacagttactgtaatatcctttgactgcattgcctatagttttcatgtagaatttttgttgagcacgagtcaagtacagtacagaagtgtctgtaaacatttctttgagtttggtaatacctaggacagccttcaaatatttagggcaggtactatgcttcaagtatgtccttagtgggataaggtcctccatggagatcatgcctttaagcactaggctgattatatcgcctattttattgagattttcgactctaagacctagtattgtaacctctaggatctgtctcgtgcactgtttaagtgatgtcctctttttcccgtcagtagagagattgtaaggggtaaatagaaatactgtgtcagcgagatcaccctcagcttccatatatatcacagaccggataactgtgtcaatgtgactagcgatgatctgggttatctgagttatgttcgctagcgtgctcaccagactctccgcacagaatggacggacgggctgtcccccggcttcaatcagcgcagtgtcaatattcttcctcaagtactttattaatcttggtaaaggactggataggatgtctgtcacacgctgccgctgtgaggtgaataacctggtcagtgtgatctcatctgatttagacaaaagcgtaccgtgccgctgcaccagagttttcgccatcctcaccacctcatgtacaaatgtaggcccgcccaggtaacccatgacaaataccaggtaacactccatatctcctcgacatgcataaccattagagagaatatatccttttgtggaacacggagcaaacaagttcatgagtagatgaaagtagtatcccattgcatacaacactttgatgattactaccccgccctcccttacagaatgcatggcaatcagagataaattgatagctagttgatctagtaagctttgattggaccctggaggaatttcaatgtcacaatgcagcaatgatacagatctgtagggcactgcagatgtaatataccccactactttatctgaggtcaggtcgctttcctctgtattttctctccataatggacggaactcttggacaaatccatctttgcatgttacctccgcctgtagattcctataaacaaccgaattcaaaaactgagttggggtcggcccgaaatgtcgttgcggggggttcatctcatttgaaaagagcgtattgtaatagatagtttcatgtggtacatgcagttcgagaagactcatgatggctccgctcccttcagctaagtataaggagttcccgtgtcttgcacatcttacctcgggtacagaaaggagatgagatgccttataccaagaggaagatgcagtccctatccctctgaacaagtatcgtactaagtagtcttcctccggatttggagatgtgagttcaggatgaatctgactaagtgtgaatgcgtcataccttgctggagcactcaaatctaactcttgcaaaaatgccgcaggttgtcgggtgaatggatctttcactcgagcaccaatatcttgcacagaagggaactctaataatggctcttgggggaacaacaacgccaggatagtatccctgtcctccctttccctgatcaaattgaggctcttccgagacatgtagtacagattagctgggaatgtaattatgttaggagacatgatagagctcacttgatcggggctaagtaatggtttcacagcatccgacagtaaatactcagtgagtattgaacatttctcttctgcagttaagcctcttattttcgggatttctcttgttgtagcaaagagtaccgtgtacagacagcataaccgggatatcagctgaagcatcttctcattcaggttatcatctaagacagatgggaacagcagatcatacttatttcctgaatataagccggagatcacacgtcgggtgcaagatactaatagttttgcagacatttcgataaaatccgtatctgcaagttggtgtgatccgtcatggttgaccaggcccactgcatgtaaccttgaatgaatgacgggatgagatattgtagctgcaatgttggaaagtagaattcctggcatattcttgtataaatcacccatatataagacaatattgtccaggcctcttactctcaggtaatagagttggtaagaacagtcgaggagcacttcaagtgctgcatattcaaataggcggaccacatctgaattctgagcttcactgatccaatttcgggtattgtcatacactattatggcgtcattctttatggaggtatcttcatcataagaaaccacagactggccaatcaacttcccgctggatattgaaagaatgttcattagctctatcgtgggatatgactccagattaagctcataactcttgaagatagctaagtcaagtctcgcaaagtctccctccgatacagggctaggatcatacataaacttatttgaggtcactgtcctcagttccggtaccaccccaagtagctcgaaaggaaccgcaacaggtgcttctctgatacagcaactaaatttactatgtaggtgcagtgtgatctcatcatatgtcctggttgttgtcattggaaagatcgattcgattagagataaacccaagagcatgatctgttggtaaaccacattcccctctttgactccttcttcagtgaacagcctttgagaatcattggatatgtgaatgtaaggtgacaccctgtagagagatgcaggggtgaatgtcatctgagttataccatcatctagtctatgttgaagattcccagccgtgggtaaaggggacagtaaccgaagatactctaagtttacattacaccgagattttgcaatcgtaagagcagcagtccaatttacttcattatccccataagcccagatcaacacggatgatgcccttagggcagcctttacatgtggcgacatatgagctatttttgcaagtgaggcagctctcctctcctgtgtctttgacccgagatatggtaccctcatcggaggattcttgctggtgtcatcggtcaattctatattgcttggaagatggaaccaagtaaattgttcatctccgctgtcacatcttgtacaccctccgcttacactaagaatctcaccctctacgagttctatcgtatcaggattagatacacccagtattttcctgcctcccgtcaaaggtgaccagcttctattccgtgcatagtctgccagtgtcagagaacacatattagaagagactaaggggtggttggatctactggaggaaaaaacatcgtctctaaacagcattgcatgcatgctagaataattgactatccgcatcagcctcttgatgcctaatggcctcctagtaagcgcaatcttaattacggtgtttgttgtgtcaacaagcccttgaatttgctttctcctacctacagagcttgcctccatgatggcatgcgcaacgcggggatgaatcacctcttgattaagcaagaattcagccaatgccttctcttctgcctcattatcctctgtgtgcactccagacaataagggatttgaacaagtttcaaataggactctttgcgtatgtttcttaagaacaatatttgggcttgcaacagtctcaaaattgaaagagtatgggtcgttgcacagactggcccaatctccattcccaggcggcctagttaagatattagtcataatgttaggactcagtaatcccactgcttctagtcgcttgatctctgcaaaagcagtagtccccgggtcaccgatatttctagtgtagagccttgagtattgaaggttactcagtccccctaattgggcaggagtcagaacatatgagtgcacaaaagagatgtcctcaatccacgactgattaagatcggggtgcgaattgttggtgatggagaactcagagtcaaagtatgtctgcacacaactcattatatagtttaaatagtaacagaagtctttgggaagcccgttctcgcatagccgtgctacagtagaggcaatgttggcacaggacattacggtgttttcactgagatcacctgacactagcactaatttagatgaatttttgaggacttgactgaggattgctccatctttgaagattcgtttgctgtatatgaagaatgtgtctgacctgatggtttcacgatccttcaaattatggccaatcaaatgattgacatgaattaattccttgaagaaattatcactggcttgatgcaactgtgtcaacaccatctccggagagtcgtctgatcttacctctctcgttactgctattacttgattatcaccctgtaccatacaggcaacacgacaatgcgatctagctgcagcaagttggattgcagcaattgagatcattgtccatagcttctggcataatccttcgataccccctctggcactgacaatatatatgtcatcattagggactcttgagaggtcacagtcagtagggtcacttggaggattgaaagggtctcctacgaacatcgtagtgtccatcagtcttaggtgaatccattcgaagaagtgaggtaggcccatcaactgattgatggcatgagcgaacaatttgattgtctgatatctccaattaagacagtacttttgcaggtcagttgttatgaaggttgcaactctccgacggttcttgcttttcggatcatgattgcggtttgaagatactctttctttacagtcagtgatacgtttcttattgctgttaaaagacagttgactcatcgctagcatactcttggtcaaggatatgctatcctgaatgactccatttccctgaaagaaaggtgcaatctgatcggctaggatcccttccgccatcacctgacagttccttaacttctttgtcagcttagcgaagatccgtccattaactttcacttccttctccttgagcgagtatgatactgccacattgtcatctctaaggtactcaagggtcgtcagatattccatctctttatatggatcaaaatcatttgactctaaaaactctatcaagaggcgattagtcgaagttgcttcttttacatgtttcttctggtcttcggagagaaggttccgcctaaacgaggcaagccaattatcgttggggtgtgcgattgccttgtcttttaggaacatgctcaggttggtgacagggtcatattctatacatggctcaaattcaagtgcagataaactcttatactctctcaacatgatatcgtgtgaaatctctgctgaatctgcatgtagttgcccaatgaccttcccatatattgtatccactttgactcgcggccacacacctgcattcttctttctgtacccgttgatgattgttcccttgaagaaagacagtacctgaaggatcatatcaaagtctaccattttcggtgcgcacatttggctcctgactgcctttgctgcaatacgggactcaagcagtgggtgaccccacagacgcaacagacacaacatctcagctgcttgattctgttctaaaccagagaatacagtagcgattgcatgagtcacggattctgctatatcattggggaggaggccaattagaatgtctttaagctcctgcaggttgaatgcgaagaaatctcctgcaaatgtacctgacggctcgagtagctggacagctccgtatgcaaatccctccattagtgatacaacatcgtagacttgattacccaagtcttttgccagagcgtctattaaccgcaaaatgtcatcaattttctctgataagcttctgagatgcaccgccgtggttgatattatgttgaccatatctctgccctccatcatatctgcatacatcaatacaagttcctgggtaagacatgtgaacttgttctcattcgtatgcgtcacaacgacaagttcaggagtgacaaagacttggcctaccttatgggttagcatcaccaatttgttggccgcagaccttgtcctagctgccaccatcagatgcctctggatctgttttatatggagccatgcaaacttggctgtggaccattttgagtgaaaccagaatgccggatccgtacgaatgctgctgaactcctctgaccgggggacattgttagaccaagatgaccccagcagtttcttctctatatgcgtacacagccttgtgaacagttctccatatctcgtgttgtgaatttggatcttcttctcaatcttccgaaatttgttggttgaatctgggacctcaatattagccagttcttctaaacacctggggtggagcactccggttattctggaattgtggttaagagtttggtgtactgcccttccgagttttatcattctctcagtatcaggagaggccgattcaagtatttttttccattgtcggctgagaatgaggtggtcgaagtcacattcatcaggaagcggtagcccagttaatttccagtaatagagtagtttgtgcttgaccaatggtgaagacaggtgtgactctggtaggataatctgatgctctgccctttcaggaccggagctcgccatgtcctacccgtattgttaaccatgagctgttttgccttgtatctcattgccacttacattttttcttaatcaagagactattgacaaggcttaatctgatcgcatgagcactggctgattgtggtcaactggcaacatggaattcgagcacgcgccggcattcggtttgattcttgatgtcgcagaagataggtgatacaatgccatctttccaactcctttataattgactcaactagccagacctggcttctctaaccccgtcatctttgaggatctcaactagtaacgggacgattctgaattctccgaagagagtattagatatttcagcaatgctgagacaataggtcttattagtcttgaccactttaaaacaagttgatgttgtgtatgctgctttggtactgcttgaactcactcgagtaatgcgactgcgggatgtgctatcgaatactgcagacgcagggttaagtcttgcttgtacaccatcaagcattgtcccgaatacccctcgcaaggtgtggtttctatagaagattaggggatatggatctgtatagactccagtaacacacgagttggggcatcttgctgaagcctggcaagggatactacctggccgagtgaaggcattgaatgtataaggactatgaagagtggctgttttgttgctgactgtcataggatataataacgcgggagagaagtatgatgaccctcgttgatacaagaaatgagatgtccctactgtgagaattctgccttcggcccccatgagtgtgactgtgttgggcggtacagtcagtaccgggtcttcgcctaaggatgttgacaccttgatagataagatagcctgctgtatgcgtttcccaccaaaccgtccaggcttatacgaagacttggccattcgaatctggtagtcttgctcatctgggcatgtgtcattgtatcgcttgtatatcacatatttcccttcctgtacagtgtcactgggtgaattgggttttaaccctccgtagactgagaaccatacgcggctgtcaataaaagatccaccccctactcctgggtagttggccacccagtccccgaataatgttgtgacatctaggtccttttcgtggtactggccgtcgaaccctaacctcccatgtaccatccgcgtagggacagctgagttataatcttcttcctctgtctccgtgactttcgagcacagcatatcacaacccaggggagttgcactcacactgcaagacttccgattttgggtgtcgtccaggttgatggaacgcagagtagaaaagaataccctccctgttgcagatgtccggagcacaccaagtgctaaatactgatatgaatgtgagtgatctctgcatccagacaatattacattatgggtgtagcagtaatgggtagcactcatgtcaaatgagggtattcgagtgcaacctgatcctgtagtaggcgccgggataaaattcagatgttcttgaaatgcagagggatagaatgatgtgacatcactagcatcatctacaatgagttctttgcctatcccccctatataatctgggtcatggataggtgccccccacccactgttgtttgcagctccattaatctgataagagagagatgttattgcgttcataattgtggtctcagtatttaacaatgccaacggagactcaagggccacttgcttatatatcctatctactacatcttgattggaaccaagtgtagatgtaatcttttcttctgccctggaaatcctagtcggtatgcctacaagatcgctaggtgtgctagcccccatgctatataaaagggaggctacagatatagccaaggtcactactgttaagaataagattgcaatccggaatatcaagcgccatgtattttttgcctctctttcatcattctctaacgcaacttggctaacggcgcggtccatgattgaggactgttgtcggtgaagcggtagaacggaggttgtgaagcctggctcgcaattgaggggcggcctctcttaccgttctacccgtatatcgtcctttggtcatctacaaccggtagttttttcttaactctctgaactgacagactaccagaactttcacacaaattactattagggaaaccttcgttcctcatctgtgttcacatttttgtagtggctctcatctgatctagagtattattcccaagccataataaggtcttttgttgcgccttttgcttgtacattaggtagcatgctagaatcaggctaagtataccaaaaacaagagatatgatagtcaaaacgatataggtaatgagagcagatgtgctagtcagtttgacattgactttgtctagttttctgttgctttcctctaacttattcaaagcattactgatcgagttgttgacattcccaagctcagttgagatatcaagattgcctgttattattacttgagaatcttgtattgagatattcttctgataagttacatcgaattccccactgagccttaaagttatcccgcctaaggataaaacattgcatgattgtttatctattagagacacggcttctccatagttttgcgatatgatacccggggggtttacacatctacatgttgtcatcttgcagttggcgatgactgaacctttgatagtcatgtatggtgtagtaagtgcgccttcggtctttgagtacatacaggccgacgtattgccgctcaagcaggaataaataccaggggacatagggaacgttactattcttgtacaatataaatctaagtcagtttctatacagtatgaggtgtcaagttcttctatcacagaaccgacctgtgtcaccacttttgggacaagtgccgaggcaaatcccctggttgtgcttacggataaggtttccaagtaggtggcacgcatattatttaggttcccgactgaaggggcagttacctgtatacccaagagttgagtctgtgagtcgtatagaatagggttaccggtgattaagccgctaccgattaatgagctgagttgattgttccctacacctaacttagtcaataagtaatccatatttccaccagctagattgtaaagtgcctgaatagtcagcttgtttaaagcaggtgaagtgatttgtggtccgaatactgtagtcaattcggttaggtacaggttgagctctacaccaacttgctgtgcaattttgatgcagtctaattcctgagctgttttattaaattggtcattaacaaactgctgcatcttcccaactgccactgctagttgcgataatccgtcagtgacctcatgcacagcctcattggttgcggcaatgctctctttaagtcggaggatgttggcagcattttgtttggcttgtatcagagctgcggccgctgttatttgtgcggcagttgcaaccccaagagccacaccgccaataatggcgcctataaggcgcccctgtctcccccctccagatgtagtcacagactcttgtatcctacggatagagtcaccaaggggggtgagcaaagtggtcaatgtcctgttgtatgcatccaagggggctttcgcacatgcctccttatccttgggcagattcgggaggagcttaactatgattgatcctgtctgggatgaggtgtatatgttgacggctttgtctcctgtaaccacaattcctgcagctgcaagaggcctgccatcaatggagtttgccggacagatgcaactcagtaccagcgcaacccggatagtcagcatcataggtgctgggttcttggtagaaggtctggagcccatcttgcacctggagggcgccaaccgggatccagaatcttctacccgtgttttttctaacttgatagacaggtaaactaactgtaaataatcaagacagattagttttttgtgtcatgatgatctgggtgagtgggcggagcgcaatctcagagacgcagcttatttcttaaaaggattgtatttggcaagggtgtgccccttctccagcttagtagaggtaacctcgtggtcggcggtcactgcgacagcgcgttgggtacctgcttggataatgattttaacgctccgcaggcacgcggtttgactccagagtatcttggccacctgaggagaagcatttgctatgggatagcaggctgtcccactgctagagaagaaaggtgccaaaagcttagtccgtgcacctcttgcttttaccaacacggaaggcccgagcacatcactgagcccgacagatagatcaaggctccttattttcttttccagcttgtcaaatgtcactttcttccccttcctatctacagtggtcataagtccaatatgcaagaagaggttagcatagtatccgctgtcagacttagacagagatttaaccaaaggactcctcgggtctacctccacattaatagtgacattgagcgcaagattgtacagactcgagccagaaaccttcaatactgcagctgggatcttgtagacatccctcttcggtaccacagtcaaggagacaaagttcaccttgtattctagggttccactcccgggaatcttctctggcgctttcacgtgcttgactgcattcactgatgagtatttgtttgccacaaccctacagctttgcagcacttggggtgcctgcactactgagaaaaccattctctcagtattagttgcactcttcttgcatgtgactatcatagtgagacaggcccttgccagctcaataaggtctccggtatttgggacgcttcctaggcagagcatcgcagcggaaagtaactcgcgcttgggtttatcatcgatcatgccgacggtggcttcttcattcccaacttgaaagatgaatccataggtggtgatgaatactgagtcctccttactatcagtccacaagtcaaggcgctggatcctatattgcggggcgatttgcttcttcccatctcctgtgtcttgtaggacgatcggaaatgctaacaggttgctagaagaatgggcagaatcaaagtacagcccaattgtcctagatgagtccatcttggcacaattggggcactccaattctacccgtattttttcttaatcttaaatgtagctagattaattacggttacgcgatcattcagtggggctgaggaagcgagagaggattgccgcttggagagttggaccttgggtccgcggatcattaggaagcattcagataggacatcacgcggtcgattgtcacggctctgatccgaaaggcgtgcagcaggatgcacagcttgatcttggtcttgtagaaatctggctcctccagagaggacttctgaggcactgtctcgctattgaagttcagggcctgcatcagctcgtcgatcacggccagcatattctgatccagaaagatctgcctcttggggtccatcagcagcttggcgttcatggtcttgaactccacctgatacatcttcaggtcctcgtagatgctagacaggcacagggccatcataaagcttgtctttctagaggccaggcaggagccgttggtgatgaagcttgtctcccggctgttcagacaggactcgttcttggtcagctccagtggcaggcaggcctccactgtggaggtcttatcctttgtgatgtcctcgtggtcgatctcctcgctggtacaagggtaaaactccagtgtctggcgggccttctgcagcatattggacacggccctcagcaggttctggctgtggtgcaggcaggggaacatgcctgggtcaggggttgccactggcagattcctagatcctcctcctcctccgccgctgcaaggcacagaggcccactcggaccaggagctagaatagtagcggtcctgggccctcacgctgatagaggcgttctttctacagatcacggtggcagatgtcttatcggtgaacactctgtccttcttctcccgcttggacttgccctgcacctgcacgcaaaaggtcagagagaaataggagtgtggtgtgctccaggtgtcagggtactcccaggacacctccacctgtctgctattcttcagaggcttcagctgcaggttcttagggggatcgggcttgatgatgtcccggataaagaaggagctggtataattctcgtacttcagcttgtgcacggcatccaccatcacttcgataggcagggactcctctgctgctggacaggcagagtcctcctggcactccacggaatactcgtactccttgttatcgccgcgcaccctctcggcgctcagggtggctgctccgcatgtcactccctgtgggtcagaggatcccctgctagacttcacagaaaatgtcagatcggtggagattgtggtcagccaccaacaggtgaaccggccgctataattcttggcctcgcaccgcaggaaggtcttgttctttggctccttctgatccttcaggatgtctgtggaccagatgccatcctccttcttgtgcagcagcagcagggagtggctcagcacctctcctcccttgtgacaggtgtactggccggcgtcgccgaactccttcacctggattgtcagggtcttgccgcttcccagcacctcggagctctgatccagtgtccaggtgatgccatcctcctcgggtgtgtcgcaggtcagcaccaccatctcgccaggggcatcgggataccagtccagctccaccacgtacacatcctttttcagttcccagattgcgaccagaggtgaggccagaaacaccagggagaaccatgagatgaccagctgctgatggcacatggtggcggttctacccgtattttttctaaccgcgggggggaactcggtgcagattccgtgtgatgccgagtggacagggacccgctacgtgtggcagtagtaattagccatttagagcaaggcgcttgattttcctgatttcctcgatcgacccggctgcatctaacttgcttaggagcttggctgaagaactcgggtgcattgggcgtgacatgatcaatgcacggacagtgtccttttccactcctatatcaggcccgcatgcagtggcgggtttaatcaattcagatggatgtggcactggttgcgaaagtttattaagtgccatttcgcctccttgtgtcacatagggagaggggtctccagggcctgaaactaaaaccgggtgagatcgggcaactgcccgtagatcactcagagatgaaatgttggcacaaccgggatccagaatcttcatcattcccaagttggcttccatgactgcaacagatgttttcagctgttggatttcggaccgcatcatagggatggaggatgtctgtttcaagacaagatctagctgatagtcaaccttacttactctctgtgatatcgcctccatcatagacatcatcgcttgcacaaagtctacaggtggctggacatgatccgcagatacaagggtattgtcttggctctgccttgatcggagagcatgaggggttgcaccagctgatagttgtgactcctcccattgtccatgatatgctgtgttcacgtctgtgccctggtttccaggggcggccttgacttggttctgcggtctttcctgactgtttccgcgactgggttgactcccctgctgttgagtcggacgttggtgattcccctcttgggggctcgaccatgggccctttttagcattggacgatttattgctgagcttgtcaagcatcaacagcagagagttgcttgctccggtcctgagctgtgtgtcgacggcttcgtctgtggcctgggtggggggctggtcggcggatgtggccggcgggctgtcatgcggggtcgtttgctcgggtgtggatggttgtttgtcagatctgtcctgtcgatcggggttgtcttgactggccggtggctggatgctcccatgcttctcccatgctgcgctcagcaccttggtcttgccttgtgggattgcactccttccaacagtctctgctggtttaccctgggctgtaattatgttgtcaatgacagttccacttgtctcaaatagctcgtcgatctctgcatctgtaaaggtggccatgtttgtcctggtctatcaggtagaggagagagcagagactcgggagacttgccctgatctctgaatatccctcttctacccgtacttttttctaattccctcggctctgttttgattgttagtgagccgcattgtgcctgtggtatctagggcgtacgtagttgtacagcagggggagggaggaaagagggggatgtttgtttgagggtgtggtcatatagagccgcaaatcgaggggtcgactacgggtgagggcattgggatgtttttgtggaagcaggctgggttttgtccatcaatacccccagtcggtgtcgttatcttgggatggcccaggagttgggggaggccccgattggggagtgccctgtgcagagtttggcgcctccctcatgctatttgctaccgctctcatcagatccaggaattgggtctccccatccccggcttctggttgcccttgcgatctattcgatccgccttgtagagcttgggaccccccctcgctaagcccagtgaggactccgacttgttgagtaggcatgtctatgctgctggtctcctcggagacccgttgggcagcagctgccaggcccctccttgctgctggggttagctttagctcggcagccatatcctcgttaatgctacttccctgagcctgagcgtactctactccaagtctccagaatgatgtgctcataaagtccctggcaaattggtatttcccagtacctttatctaggactgatgccatacccatggcaaaggagtaaagttgtgcatactcggcaggcgcaaagctcatctggtcactatcaccaagtaatgtcatgtacggcgcattatctcctttcatccgatacaaacgcatgagctgcttcatcttctggatgtcgcctgagaggctactaagtgcaagggctgatgtcttggtgttgattccgtacttgagtgtcaagaagaatgcagtaagcccggtattcctgatgtatgagtctacgtcccctaccaggttataataagtagaggtaccacctgccgtgttgcggcctctcttgagctcgctaaccaaaaagatgcggactgcaagagactgtctgatcgtgagttggattgtgctcctgcatacggggtagaggatgtatttcttttggaccctgccttgctgcatatacttattgattcgccttgtttccgactcatctgcagtctcatacgcagtcatggcttttgctactgtgacccatacttgagcctggatagagaggatcctctccagggtatcggtgatgtcttctggtgcatcatcttcggccccggctgtgacgaacggggttccgttgctgcatgcccgagggagagatcctgctatcatcgcaaatctctgtgctctctcttcagacactccactcctattgttgaactggggcgtgccgttggcaaagccatcaatctcaagcacggccaatgtggcttcattctgtttccctgcaagggcaacatggttcctcattacctgtgagtgggagcataaaagagatatgagagcaccttgcctgagtggtttgttggcatcttcgctaacagcaatccggaggcagaataccacaaagctccatctatcttctgggtcatcactgttaagagtgaataccgggacgtctacttttaaggtactccctttttctccccctccatgagctccattggggcgagtctgagccgcgaggagctgttcgtactcatcaaatacggaagacatgttggcagaaggctttctcgagtttgtgcttcgggctcgcactcgagattcacaccttctacccgtgcgacttcaattgctccttcgccttttatcgtaactcacggattctctgtttggt

SEQ ID NO: 53 - DNA, 1599 bp, synthetic construct
  source 1..1599: mol_type = other DNA; note = Description of Artificial Sequence: Synthetic polynucleotide
atgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaatgcttcctaa

SEQ ID NO: 54 - DNA, 984 bp, synthetic construct
  source 1..984: mol_type = other DNA; note = Description of Artificial Sequence: Synthetic polynucleotide
atgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagc

SEQ ID NO: 55 - DNA, 594 bp, synthetic construct
  source 1..594: mol_type = other DNA; note = Description of Artificial Sequence: Synthetic polynucleotide
aggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaatgcttcctaa

SEQ ID NO: 56 - protein, 7 aa, synthetic construct
  source 1..7: note = Description of Artificial Sequence: Synthetic peptide
HNRTKSF

SEQ ID NO: 57 - DNA, 918 bp, Homo sapiens
  source 1..918: mol_type = other DNA
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 58 - protein, 7 aa, synthetic construct
  source 1..7: note = Description of Artificial Sequence: Synthetic peptide
HNKTKSF

SEQ ID NO: 59 - DNA, 918 bp, Homo sapiens
  source 1..918: mol_type = other DNA
atctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagc

SEQ ID NO: 60 - RNA, 16824 nt, synthetic construct
  source 1..16824: mol_type = other RNA; note = Description of Artificial Sequence: Synthetic polynucleotide
accaaacaaagatttggtgaatgacgagactacactcaagaataattgtgcgcaacctttttttaagacatttatttgagttcgaattcgagtcctaaggagtcagggttcaacttttttctaacataatatgattaaatcaacaagaatacaattggccaaaaaaggagcctattaatatgtgattttcgttaagagtcacagttactgtaatatcctttgactgcattgcctatagttttcatgtagaatttttgttgagcacgagtcaagtacagtacagaagtgtctgtaaacatttctttgagtttggtaatacctaggacagccttcaaatatttagggcaggtactatgcttcaagtatgtccttagtgggataaggtcctccatggagatcatgcctttaagcactaggctgattatatcgcctattttattgagattttcgactctaagacctagtattgtaacctctaggatctgtctcgtgcactgtttaagtgatgtcctctttttcccgtcagtagagagattgtaaggggtaaatagaaatactgtgtcagcgagatcaccctcagcttccatatatatcacagaccggataactgtgtcaatgtgactagcgatgatctgggttatctgagttatgttcgctagcgtgctcaccagactctccgcacagaatggacggacgggctgtcccccggcttcaatcagcgcagtgtcaatattcttcctcaagtactttattaatcttggtaaaggactggataggatgtctgtcacacgctgccgctgtgaggtgaataacctggtcagtgtgatctcatctgatttagacaaaagcgtaccgtgccgctgcaccagagttttcgccatcctcaccacctcatgtacaaatgtaggcccgcccaggtaacccatgacaaataccaggtaacactccatatctcctcgacatgcataaccattagagagaatatatccttttgtggaacacggagcaaacaagttcatgagtagatgaaagtagtatcccattgcatacaacactttgatgattactaccccgccctcccttacagaatgcatggcaatcagagataaattgatagctagttgatctagtaagctttgattggaccctggaggaatttcaatgtcacaatgcagcaatgatacagatctgtagggcactgcagatgtaatataccccactactttatctgaggtcaggtcgctttcctctgtattttctctccataatggacggaactcttggacaaatccatctttgcatgttacctccgcctgtagattcctataaacaaccgaattcaaaaactgagttggggtcggcccgaaatgtcgttgcggggggttcatctcatttgaaaagagcgtattgtaatagatagtttcatgtggtacatgcagttcgagaagactcatgatggctccgctcccttcagctaagtataaggagttcccgtgtcttgcacatcttacctcgggtacagaaaggagatgagatgccttataccaagaggaagatgcagtccctatccctctgaacaagtatcgtactaagtagtcttcctccggatttggagatgtgagttcaggatgaatctgactaagtgtgaatgcgtcataccttgctggagcactcaaatctaactcttgcaaaaatgccgcaggttgtcgggtgaatggatctttcactcgagcaccaatatcttgcacagaagggaactctaataatggctcttgggggaacaacaacgccaggatagtatccctgtcctccctttccctgatcaaattgaggctcttccgagacatgtagtacagattagctgggaatgtaattatgttaggagacatgatagagctcacttgatcggggctaagtaatggtttcacagcatccgacagtaaatactcagtgagtattgaacatttctcttctgcagttaagcctcttattttcgggatttctcttgttgtagcaaagagtaccgtgtacagacagcataaccgggatatcagctgaagcatcttctcattcaggttatcatctaagacagatgggaacagcagatcatacttatttcctgaatataagccggagatcacacgtcgggtgcaagatactaatagttttgcagacatttcgataaaatccgtatctgcaagttggtgtgatccgtcatggttgaccaggcccactgcatgtaaccttgaatgaatgacgggatgagatattgtagctgcaatgttggaaagtagaattcctggcatattcttgtataaatcacccatatataagacaatattgtccaggcctcttactctcaggtaatagagttggtaagaacagtcgaggagcacttcaagtgctgcatattcaaataggcggaccacatctgaattctgagcttcactgatccaatttcgggtattgtcatacactattatggcgtcattctttatggaggtatcttcatcataagaaaccacagactggccaatcaacttcccgctggatattgaaagaatgttcattagctctatcgtgggatatgactccagattaagctcataactcttgaagatagctaagtcaagtctcgcaaagtctccctccgatacagggctaggatcatacataaacttatttgaggtcactgtcctcagttccggtaccaccccaagtagctcgaaaggaaccgcaacaggtgcttctctgatacagcaactaaatttactatgtaggtgcagtgtgatctcatcatatgtcctggttgttgtcattggaaagatcgattcgattagagataaacccaagagcatgatctgttggtaaaccacattcccctctttgactccttcttcagtgaacagcctttgagaatcattggatatgtgaatgtaaggtgacaccctgtagagagatgcaggggtgaatgtcatctgagttataccatcatctagtctatgttgaagattcccagccgtgggtaaaggggacagtaaccgaagatactctaagtttacattacaccgagattttgcaatcgtaagagcagcagtccaatttacttcattatccccataagcccagatcaacacggatgatgcccttagggcagcctttacatgtggcgacatatgagctatttttgcaagtgaggcagctctcctctcctgtgtctttgacccgagatatggtaccctcatcggaggattcttgctggtgtcatcggtcaattctatattgcttggaagatggaaccaagtaaattgttcatctccgctgtcacatcttgtacaccctccgcttacactaagaatctcaccctctacgagttctatcgtatcaggattagatacacccagtattttcctgcctcccgtcaaaggtgaccagcttctattccgtgcatagtctgccagtgtcagagaacacatattagaagagactaaggggtggttggatctactggaggaaaaaacatcgtctctaaacagcattgcatgcatgctagaataattgactatccgcatcagcctcttgatgcctaatggcctcctagtaagcgcaatcttaattacggtgtttgttgtgtcaacaagcccttgaatttgctttctcctacctacagagcttgcctccatgatggcatgcgcaacgcggggatgaatcacctcttgattaagcaagaattcagccaatgccttctcttctgcctcattatcctctgtgtgcactccagacaataagggatttgaacaagtttcaaataggactctttgcgtatgtttcttaagaacaatatttgggcttgcaacagtctcaaaattgaaagagtatgggtcgttgcacagactggcccaatctccattcccaggcggcctagttaagatattagtcataatgttaggactcagtaatcccactgcttctagtcgcttgatctctgcaaaagcagtagtccccgggtcaccgatatttctagtgtagagccttgagtattgaaggttactcagtccccctaattgggcaggagtcagaacatatgagtgcacaaaagagatgtcctcaatccacgactgattaagatcggggtgcgaattgttggtgatggagaactcagagtcaaagtatgtctgcacacaactcattatatagtttaaatagtaacagaagtctttgggaagcccgttctcgcatagccgtgctacagtagaggcaatgttggcacaggacattacggtgttttcactgagatcacctgacactagcactaatttagatgaatttttgaggacttgactgaggattgctccatctttgaagattcgtttgctgtatatgaagaatgtgtctgacctgatggtttcacgatccttcaaattatggccaatcaaatgattgacatgaattaattccttgaagaaattatcactggcttgatgcaactgtgtcaacaccatctccggagagtcgtctgatcttacctctctcgttactgctattacttgattatcaccctgtaccatacaggcaacacgacaatgcgatctagctgcagcaagttggattgcagcaattgagatcattgtccatagcttctggcataatccttcgataccccctctggcactgacaatatatatgtcatcattagggactcttgagaggtcacagtcagtagggtcacttggaggattgaaagggtctcctacgaacatcgtagtgtccatcagtcttaggtgaatccattcgaagaagtgaggtaggcccatcaactgattgatggcatgagcgaacaatttgattgtctgatatctccaattaagacagtacttttgcaggtcagttgttatgaaggttgcaactctccgacggttcttgcttttcggatcatgattgcggtttgaagatactctttctttacagtcagtgatacgtttcttattgctgttaaaagacagttgactcatcgctagcatactcttggtcaaggatatgctatcctgaatgactccatttccctgaaagaaaggtgcaatctgatcggctaggatcccttccgccatcacctgacagttccttaacttctttgtcagcttagcgaagatccgtccattaactttcacttccttctccttgagcgagtatgatactgccacattgtcatctctaaggtactcaagggtcgtcagatattccatctctttatatggatcaaaatcatttgactctaaaaactctatcaagaggcgattagtcgaagttgcttcttttacatgtttcttctggtcttcggagagaaggttccgcctaaacgaggcaagccaattatcgttggggtgtgcgattgccttgtcttttaggaacatgctcaggttggtgacagggtcatattctatacatggctcaaattcaagtgcagataaactcttatactctctcaacatgatatcgtgtgaaatctctgctgaatctgcatgtagttgcccaatgaccttcccatatattgtatccactttgactcgcggccacacacctgcattcttctttctgtacccgttgatgattgttcccttgaagaaagacagtacctgaaggatcatatcaaagtctaccattttcggtgcgcacatttggctcctgactgcctttgctgcaatacgggactcaagcagtgggtgaccccacagacgcaacagacacaacatctcagctgcttgattctgttctaaaccagagaatacagtagcgattgcatgagtcacggattctgctatatcattggggaggaggccaattagaatgtctttaagctcctgcaggttgaatgcgaagaaatctcctgcaaatgtacctgacggctcgagtagctggacagctccgtatgcaaatccctccattagtgatacaacatcgtagacttgattacccaagtcttttgccagagcgtctattaaccgcaaaatgtcatcaattttctctgataagcttctgagatgcaccgccgtggttgatattatgttgaccatatctctgccctccatcatatctgcatacatcaatacaagttcctgggtaagacatgtgaacttgttctcattcgtatgcgtcacaacgacaagttcaggagtgacaaagacttggcctaccttatgggttagcatcaccaatttgttggccgcagaccttgtcctagctgccaccatcagatgcctctggatctgttttatatggagccatgcaaacttggctgtggaccattttgagtgaaaccagaatgccggatccgtacgaatgctgctgaactcctctgaccgggggacattgttagaccaagatgaccccagcagtttcttctctatatgcgtacacagccttgtgaacagttctccatatctcgtgttgtgaatttggatcttcttctcaatcttccgaaatttgttggttgaatctgggacctcaatattagccagttcttctaaacacctggggtggagcactccggttattctggaattgtggttaagagtttggtgtactgcccttccgagttttatcattctctcagtatcaggagaggccgattcaagtatttttttccattgtcggctgagaatgaggtggtcgaagtcacattcatcaggaagcggtagcccagttaatttccagtaatagagtagtttgtgcttgaccaatggtgaagacaggtgtgactctggtaggataatctgatgctctgccctttcaggaccggagctcgccatgtcctacccgtattgttaaccatgagctgttttgccttgtatctcattgccacttacattttttcttaatcaagagactattgacaaggcttaatctgatcgcatgagcactggctgattgtggtcaactggcaacatggaattcgagcacgcgccggcattcggtttgattcttgatgtcgcagaagataggtgatacaatgccatctttccaactcctttataattgactcaactagccagacctggcttctctaaccccgtcatctttgaggatctcaactagtaacgggacgattctgaattctccgaagagagtattagatatttcagcaatgctgagacaataggtcttattagtcttgaccactttaaaacaagttgatgttgtgtatgctgctttggtactgcttgaactcactcgagtaatgcgactgcgggatgtgctatcgaatactgcagacgcagggttaagtcttgcttgtacaccatcaagcattgtcccgaatacccctcgcaaggtgtggtttctatagaagattaggggatatggatctgtatagactccagtaacacacgagttggggcatcttgctgaagcctggcaagggatactacctggccgagtgaaggcattgaatgtataaggactatgaagagtggctgttttgttgctgactgtcataggatataataacgcgggagagaagtatgatgaccctcgttgatacaagaaatgagatgtccctactgtgagaattctgccttcggcccccatgagtgtgactgtgttgggcggtacagtcagtaccgggtcttcgcctaaggatgttgacaccttgatagataagatagcctgctgtatgcgtttcccaccaaaccgtccaggcttatacgaagacttggccattcgaatctggtagtcttgctcatctgggcatgtgtcattgtatcgcttgtatatcacatatttcccttcctgtacagtgtcactgggtgaattgggttttaaccctccgtagactgagaaccatacgcggctgtcaataaaagatccaccccctactcctgggtagttggccacccagtccccgaataatgttgtgacatctaggtccttttcgtggtactggccgtcgaaccctaacctcccatgtaccatccgcgtagggacagctgagttataatcttcttcctctgtctccgtgactttcgagcacagcatatcacaacccaggggagttgcactcacactgcaagacttccgattttgggtgtcgtccaggttgatggaacgcagagtagaaaagaataccctccctgttgcagatgtccggagcacaccaagtgctaaatactgatatgaatgtgagtgatctctgcatccagacaatattacattatgggtgtagcagtaatgggtagcactcatgtcaaatgagggtattcgagtgcaacctgatcctgtagtaggcgccgggataaaattcagatgttcttgaaatgcagagggatagaatgatgtgacatcactagcatcatctacaatgagttctttgcctatcccccctatataatctgggtcatggataggtgccccccacccactgttgtttgcagctccattaatctgataagagagagatgttattgcgttcataattgtggtctcagtatttaacaatgccaacggagactcaagggccacttgcttatatatcctatctactacatcttgattggaaccaagtgtagatgtaatcttttcttctgccctggaaatcctagtcggtatgcctacaagatcgctaggtgtgctagcccccatgctatataaaagggaggctacagatatagccaaggtcactactgttaagaataagattgcaatccggaatatcaagcgccatgtattttttgcctctctttcatcattctctaacgcaacttggctaacggcgcggtccatgattgaggactgttgtcggtgaagcggtagaacggaggttgtgaagcctggctcgcaattgaggggcggcctctcttaccgttctacccgtatatcgtcctttggtcatctacaaccggtagttttttcttaactctctgaactgacagactaccagaactttcacacaaattactattagggaaaccttcgttcctcatctgtgttcacatttttgtagtggctctcatctgatctagagtattattcccaagccataataaggtcttttgttgcgccttttgcttgtacattaggtagcatgctagaatcaggctaagtataccaaaaacaagagatatgatagtcaaaacgatataggtaatgagagcagatgtgctagtcagtttgacattgactttgtctagttttctgttgctttcctctaacttattcaaagcattactgatcgagttgttgacattcccaagctcagttgagatatcaagattgcctgttattattacttgagaatcttgtattgagatattcttctgataagttacatcgaattccccactgagccttaaagttatcccgcctaaggataaaacattgcatgattgtttatctattagagacacggcttctccatagttttgcgatatgatacccggggggtttacacatctacatgttgtcatcttgcagttggcgatgactgaacctttgatagtcatgtatggtgtagtaagtgcgccttcggtctttgagtacatacaggccgacgtattgccgctcaagcaggaataaataccaggggacatagggaacgttactattcttgtacaatataaatctaagtcagtttctatacagtatgaggtgtcaagttcttctatcacagaaccgacctgtgtcaccacttttgggacaagtgccgaggcaaatcccctggttgtgcttacggataaggtttccaagtaggtggcacgcatattatttaggttcccgactgaaggggcagttacctgtatacccaagagttgagtctgtgagtcgtatagaatagggttaccggtgattaagccgctaccgattaatgagctgagttgattgttccctacacctaacttagtcaataagtaatccatatttccaccagctagattgtaaagtgcctgaatagtcagcttgtttaaagcaggtgaagtgatttgtggtccgaatactgtagtcaattcggttaggtacaggttgagctctacaccaacttgctgtgcaattttgatgcagtctaattcctgagctgttttattaaattggtcattaacaaactgctgcatcttcccaactgccactgctagttgcgataatccgtcagtgacctcatgcacagcctcattggttgcggcaatgctctctttaagtcggaggatgttggcagcattttgtttggcttgtatcagagctgcggccgctgttatttgtgcggcagttgcaaccccaagagccacaccgccaataatggcgcctataaggcgcccctgtctcccccctccagatgtagtcacagactcttgtatcctacggatagagtcaccaaggggggtgagcaaagtggtcaatgtcctgttgtatgcatccaagggggctttcgcacatgcctccttatccttgggcagattcgggaggagcttaactatgattgatcctgtctgggatgaggtgtatatgttgacggctttgtctcctgtaaccacaattcctgcagctgcaagaggcctgccatcaatggagtttgccggacagatgcaactcagtaccagcgcaacccggatagtcagcatcataggtgctgggttcttggtagaaggtctggagcccatcttgcacctggagggcgccaaccgggatccagaatcttctacccgtgttttttctaacttgatagacaggtaaactaactgtaaataatcaagacagattagttttttgtgtcatgatgatctgggtgagtgggcggagcgcaatctcagagacgcagcttatttcttaaaaggattgtatttggcaagggtgtgccccttctccagcttagtagaggtaacctcgtggtcggcggtcactgcgacagcgcgttgggtacctgcttggataatgattttaacgctccgcaggcacgcggtttgactccagagtatcttggccacctgaggagaagcatttgctatgggatagcaggctgtcccactgctagagaagaaaggtgccaaaagcttagtccgtgcacctcttgcttttaccaacacggaaggcccgagcacatcactgagcccgacagatagatcaaggctccttattttcttttccagcttgtcaaatgtcactttcttccccttcctatctacagtggtcataagtccaatatgcaagaagaggttagcatagtatccgctgtcagacttagacagagatttaaccaaaggactcctcgggtctacctccacattaatagtgacattgagcgcaagattgtacagactcgagccagaaaccttcaatactgcagctgggatcttgtagacatccctcttcggtaccacagtcaaggagacaaagttcaccttgtattctagggttccactcccgggaatcttctctggcgctttcacgtgcttgactgcattcactgatgagtatttgtttgccacaaccctacagctttgcagcacttggggtgcctgcactactgagaaaaccattctctcagtattagttgcactcttcttgcatgtgactatcatagtgagacaggcccttgccagctcaataaggtctccggtatttgggacgcttcctaggcagagcatcgcagcggaaagtaactcgcgcttgggtttatcatcgatcatgccgacggtggcttcttcattcccaacttgaaagatgaatccataggtggtgatgaatactgagtcctccttactatcagtccacaagtcaaggcgctggatcctatattgcggggcgatttgcttcttcccatctcctgtgtcttgtaggacgatcggaaatgctaacaggttgctagaagaatgggcagaatcaaagtacagcccaattgtcctagatgagtccatcttggcacaattggggcactccaattctacccgtattttttcttaatcttaaatgtagctagattaattacggttacgcgatcattcagtggggctgaggaagcgagagaggattgccgcttggagagttggaccttgggtccgcggatcattaggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctagatccgccgccaccgccaccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcggttctacccgtattttttctaaccgcgggggggaactcggtgcagattccgtgtgatgccgagtggacagggacccgctacgtgtggcagtagtaattagccatttagagcaaggcgcttgattttcctgatttcctcgatcgacccggctgcatctaacttgcttaggagcttggctgaagaactcgggtgcattgggcgtgacatgatcaatgcacggacagtgtccttttccactcctatatcaggcccgcatgcagtggcgggtttaatcaattcagatggatgtggcactggttgcgaaagtttattaagtgccatttcgcctccttgtgtcacatagggagaggggtctccagggcctgaaactaaaaccgggtgagatcgggcaactgcccgtagatcactcagagatgaaatgttggcacaaccgggatccagaatcttcatcattcccaagttggcttccatgactgcaacagatgttttcagctgttggatttcggaccgcatcatagggatggaggatgtctgtttcaagacaagatctagctgatagtcaaccttacttactctctgtgatatcgcctccatcatagacatcatcgcttgcacaaagtctacaggtggctggacatgatccgcagatacaagggtattgtcttggctctgccttgatcggagagcatgaggggttgcaccagctgatagttgtgactcctcccattgtccatgatatgctgtgttcacgtctgtgccctggtttccaggggcggccttgacttggttctgcggtctttcctgactgtttccgcgactgggttgactcccctgctgttgagtcggacgttggtgattcccctcttgggggctcgaccatgggccctttttagcattggacgatttattgctgagcttgtcaagcatcaacagcagagagttgcttgctccggtcctgagctgtgtgtcgacggcttcgtctgtggcctgggtggggggctggtcggcggatgtggccggcgggctgtcatgcggggtcgtttgctcgggtgtggatggttgtttgtcagatctgtcctgtcgatcggggttgtcttgactggccggtggctggatgctcccatgcttctcccatgctgcgctcagcaccttggtcttgccttgtgggattgcactccttccaacagtctctgctggtttaccctgggctgtaattatgttgtcaatgacagttccacttgtctcaaatagctcgtcgatctctgcatctgtaaaggtggccatgtttgtcctggtctatcaggtagaggagagagcagagactcgggagacttgccctgatctctgaatatccctcttctacccgtacttttttctaattccctcggctctgttttgattgttagtgagccgcattgtgcctgtggtatctagggcgtacgtagttgtacagcagggggagggaggaaagagggggatgtttgtttgagggtgtggtcatatagagccgcaaatcgaggggtcgactacgggtgagggcattgggatgtttttgtggaagcaggctgggttttgtccatcaatacccccagtcggtgtcgttatcttgggatggcccaggagttgggggaggccccgattggggagtgccctgtgcagagtttggcgcctccctcatgctatttgctaccgctctcatcagatccaggaattgggtctccccatccccggcttctggttgcccttgcgatctattcgatccgccttgtagagcttgggaccccccctcgctaagcccagtgaggactccgacttgttgagtaggcatgtctatgctgctggtctcctcggagacccgttgggcagcagctgccaggcccctccttgctgctggggttagctttagctcggcagccatatcctcgttaatgctacttccctgagcctgagcgtactctactccaagtctccagaatgatgtgctcataaagtccctggcaaattggtatttcccagtacctttatctaggactgatgccatacccatggcaaaggagtaaagttgtgcatactcggcaggcgcaaagctcatctggtcactatcaccaagtaatgtcatgtacggcgcattatctcctttcatccgatacaaacgcatgagctgcttcatcttctggatgtcgcctgagaggctactaagtgcaagggctgatgtcttggtgttgattccgtacttgagtgtcaagaagaatgcagtaagcccggtattcctgatgtatgagtctacgtcccctaccaggttataataagtagaggtaccacctgccgtgttgcggcctctcttgagctcgctaaccaaaaagatgcggactgcaagagactgtctgatcgtgagttggattgtgctcctgcatacggggtagaggatgtatttcttttggaccctgccttgctgcatatacttattgattcgccttgtttccgactcatctgcagtctcatacgcagtcatggcttttgctactgtgacccatacttgagcctggatagagaggatcctctccagggtatcggtgatgtcttctggtgcatcatcttcggccccggctgtgacgaacggggttccgttgctgcatgcccgagggagagatcctgctatcatcgcaaatctctgtgctctctcttcagacactccactcctattgttgaactggggcgtgccgttggcaaagccatcaatctcaagcacggccaatgtggcttcattctgtttccctgcaagggcaacatggttcctcattacctgtgagtgggagcataaaagagatatgagagcaccttgcctgagtggtttgttggcatcttcgctaacagcaatccggaggcagaataccacaaagctccatctatcttctgggtcatcactgttaagagtgaataccgggacgtctacttttaaggtactccctttttctccccctccatgagctccattggggcgagtctgagccgcgaggagctgttcgtactcatcaaatacggaagacatgttggcagaaggctttctcgagtttgtgcttcgggctcgcactcgagattcacaccttctacccgtgcgacttcaattgctccttcgccttttatcgtaactcacggattctctgtttggt

SEQ ID NO: 61 - DNA, 1533 bp, synthetic construct
  source 1..1533: mol_type = other DNA; note = Description of Artificial Sequence: Synthetic polynucleotide
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 62 - DNA, 16824 bp, synthetic construct
  source 1..16824: mol_type = other DNA; note = Description of Artificial Sequence: Synthetic polynucleotide
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaaccgccaccatgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaatgcttcctaatgatccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 63 - DNA, 1533 bp, synthetic construct
  source 1..1533: mol_type = other DNA; note = Description of Artificial Sequence: Synthetic polynucleotide
atctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaatgcttcctaa

SEQ ID NO: 64 - DNA, 984 bp, Homo sapiens
  source 1..984: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 65 - DNA, 594 bp, Homo sapiens
  source 1..594: mol_type = other DNA
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 66 - DNA, 1599 bp, synthetic construct
  source 1..1599: mol_type = other DNA; note = Description of Artificial Sequence: Synthetic polynucleotide
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 67 - DNA, 16824 bp, synthetic construct
  source 1..16824: mol_type = other DNA; note = Description of Artificial Sequence: Synthetic polynucleotide
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaaccgccaccatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatgatccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 68 - DNA, 1533 bp, synthetic construct
  source 1..1533: mol_type = other DNA; note = Description of Artificial Sequence: Synthetic polynucleotide
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 69 - protein, 7 aa, synthetic construct
  source 1..7: note = Description of Artificial Sequence: Synthetic peptide
HNRMKSF

SEQ ID NO: 70 - protein, 7 aa, synthetic construct
  source 1..7: note = Description of Artificial Sequence: Synthetic peptide
HNKMSFI

SEQ ID NO: 71 - protein, 8 aa, synthetic construct
  source 1..8: note = Description of Artificial Sequence: Synthetic peptide
HNRTKRFI

SEQ ID NO: 72 - protein, 6 aa, Newcastle disease virus
GRQGRL

(19) United States
(12) Patent Application Publication
(10) Pub. No.: US 2025/0127879 A1
(43) Pub. Date: Apr. 24, 2025

(54) NEWCASTLE DISEASE VIRUSES AND USES THEREOF

(71) Applicants: ICAHN SCHOOL OF MEDICINE AT MOUNT SINAI, New York, NY (US); MEMORIAL SLOAN KETTERING CANCER CENTER, New York, NY (US); MERCK SHARP & DOHME LLC, Rahway, NJ (US)

(72) Inventors: Peter PALESE, New York, NY (US); Adolfo GARCIA-SASTRE, New York, NY (US); Dmitriy ZAMARIN, New York, NY (US); Svetlana SADEKOVA, Mountain View, CA (US); Rachel Allison ALTURA, Belle Mead, NJ (US); Uyen PHAN, San Jose, CA (US); Brian B. HAINES, Jamaica Plain, MA (US); Jedd D. WOLCHOK, New York, NY (US)

(73) Assignees: ICAHN SCHOOL OF MEDICINE AT MOUNT SINAI, New York, NY (US); MEMORIAL SLOAN KETTERING CANCER CENTER, New York, NY (US); MERCK SHARP & DOHME LLC, Rahway, NJ (US)

(21) Appl. No.: 18/737,072

(22) Filed: Jun. 7, 2024

Related U.S. Application Data
(62) Division of application No. 16/611,813, filed on Nov. 7, 2019, now Pat. No. 12,042,534.
(60) Provisional application No. 62/507,690, filed on May 17, 2017.
(60) Provisional application No. 62/505,759, filed on May 12, 2017.

(51) Int. Cl. A61K 39/17; A61K 35/768; A61K 39/00; A61K 39/395; A61P 35/00; C07K 14/54; C07K 16/28; C12N 15/86

(52) U.S. Cl. CPC A61K 39/17; A61K 35/768; A61K 39/0005; A61K 39/3955; A61P 35/00; C07K 14/5434; C07K 16/28; C12N 15/86

(57) ABSTRACT

Described herein are chimeric Newcastle disease viruses comprising a packaged genome comprising a transgene encoding interleukin-12. The chimeric Newcastle disease viruses and compositions thereof are useful in combination with an antagonist of programmed cell death protein 1 (“PD-1”) or a ligand thereof in the treatment of cancer. In particular, described herein are methods for treating cancer comprising administering the chimeric Newcastle disease viruses in combination with an antagonist of PD-1 or a ligand thereof, wherein the chimeric Newcastle disease virus comprises a packaged genome comprising a transgene encoding interleukin-12.

This application claims the benefit of priority of U.S. provisional patent application No. 62/505,759, filed May 12, 2017, and U.S. provisional patent application No. 62/507,690, filed May 17, 2017, each of which is incorporated by reference herein in its entirety.

This invention was made with government support under T32 CA009207 and HHSN26620070010C awarded by the National Institutes of Health. The government has certain rights in the invention.

The instant application contains a Sequence Listing which has been submitted electronically in ASCII format and is hereby incorporated by reference in its entirety. Said ASCII copy, created on May 4, 2018, is named 6923-272-228_SL.txt and is 263,022 bytes in size.

### 1. INTRODUCTION

Described herein are methods for treating cancer, comprising administering to a subject chimeric Newcastle disease virus (“NDV”) (or a composition comprising such a chimeric NDV) and an antagonist of programmed cell death protein 1 (“PD-1”) or a ligand thereof (or a composition comprising such an antagonist), wherein the chimeric NDV comprises a packaged genome comprising a transgene encoding interleukin-12 (“IL-12”). In a specific aspect, described herein are methods for treating cancer comprising administering a chimeric Newcastle disease virus (or a composition comprising such a chimeric NDV) and an anti-PD-1 antibody that blocks the interaction between PD-1 and a ligand thereof (e.g., PD-L1, PD-L2 or both PD-L1 and PD-L2) antibody (or a composition comprising such an antagonist), wherein the chimeric NDV comprises a packaged genome comprising a transgene encoding IL-12. In another aspect, described herein are a chimeric Newcastle disease virus for use in a method for treating cancer in a subject, wherein the chimeric NDV comprises a packaged genome comprising a transgene encoding IL-12, wherein the method further comprises administering an anti-PD-1 antibody that blocks the interaction between PD-1 and a ligand thereof (e.g., either PD-L1, PD-L2, or both) antibody.

### 2. BACKGROUND

### 2.1 Newcastle Disease Virus

Newcastle disease virus (NDV) is a member of the Avulavirus genus in the Paramyxoviridae family, which has been shown to infect a number of avian species (Alexander, DJ (1988). Newcastle disease, Newcastle disease virus—an avian paramyxovirus. Kluwer Academic Publishers: Dordrecht, The Netherlands. pp 1-22). NDV possesses a single-stranded RNA genome in negative sense and does not undergo recombination with the host genome or with other viruses (Alexander, DJ (1988). Newcastle disease, Newcastle disease virus—an avian paramyxovirus. Kluwer Academic Publishers: Dordrecht, The Netherlands. pp 1-22). The genomic RNA contains genes in the order of 3′-NP-P-M-F-HN-L-5′, described in further detail below. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding/fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication. The V protein has been shown to inhibit interferon-alpha and to contribute to the virulence of NDV (Huang et al. (2003). Newcastle disease virus V protein is associated with viral pathogenesis and functions as an Alpha Interferon Antagonist. Journal of Virology 77: 8676-8685).

Naturally-occurring NDV has been reported to be an effective oncolytic agent in a variety of animal tumor models (Sinkovics, J G, and Horvath, J C (2000). Newcastle disease virus (NDV): brief history of its oncolytic strains. J Clin Virol 16: 1-15; Zamarin et al., 2009; Mol Ther 17: 697; Elankumaran et al., 2010; J Virol 84: 3835; Schirrmacher et al., 2009; Methods Mol Biol 542: 565; Bart et al., 1973; Nat New Biol 245: 229). Naturally-occurring strains of NDV have been used in multiple clinical trials against advanced human cancers (Sinkovics, J G, and Horvath, J C (2000). Newcastle disease virus (NDV): brief history of its oncolytic strains. J Clin Virol 16: 1-15; Lorence et al. (2007). Phase 1 clinical experience using intravenous administration of PV701, an oncolytic Newcastle disease virus. Curr Cancer Drug Targets 7: 157-167; Hotte et al. (2007). An optimized clinical regimen for the oncolytic virus PV701. Clin Cancer Res 13: 977-985; Freeman et al. (2006). Phase I/II trial of intravenous NDV-HUJ oncolytic virus in recurrent glioblastoma multiforme. Mol Ther 13: 221-228; Pecora et al. (2002). Phase I trial of intravenous administration of PV701, an oncolytic virus, in patients with advanced solid cancers. J Clin Oncol 20: 2251-2266; Csatary et al. (2004). MTH-68/H oncolytic viral treatment in human high-grade gliomas. J Neurooncol 67: 83-93). However, the success of naturally-occurring strains of NDV in these clinical trials for advanced human cancers was only marginal (Hotte et al. (2007). An optimized clinical regimen for the oncolytic virus PV701. Clin Cancer Res 13: 977-985; Freeman el al. (2006). Phase I/II trial of intravenous NDV-HUJ oncolytic virus in recurrent glioblastoma multiforme. Mol Ther 13: 221-228; Pecora et al. (2002). Phase I trial of intravenous administration of PV701, an oncolytic virus, in patients with advanced solid cancers. J Clin Oncol 20: 2251-2266). As such, there remains a need for NDV-based therapies useful in the treatment of cancer, especially advanced cancer.

### 2.2 PD-1 Antagonists

Anti-PD-1-blocking antibodies and anti-PD-L1 blocking antibodies have been approved for treating certain types of cancer. For example, pembrolizumab has been approved for treatment of several types of cancer, including (1) refractory classical Hodgkin lymphoma, (2) recurrent or metastatic head and neck squamous cell cancer, (3) unresectable or metastatic melanoma, (4) locally or advanced or metastatic urothelial carcinoma, (5) recurrent locally advanced or metastatic gastric or gastroesophageal adenocarcinoma with tumors expressing programmed death-ligand 1 (“PD-L1”), (6) unresectable or metastatic, microsatellite instability-high cancer or mismatch repair deficient solid tumors that have progressed following prior treatment and who have no satisfactory alternative treatment options, or colorectal cancer that has progessed following treatment with a fluoropyrimidine, oxaliplatin and irinotecan, and (7) metastatic non-small cell lung cancers having tumors which express PD-L1; and nivolumab has been approved for the treatment of different types of cancer, including unresectable or metastatic melanoma. Although these therapies have shown some success, there remains a need for therapies to treat cancer.

### 3. SUMMARY

In one aspect the present disclosure provides chimeric NDV comprising a packaged genome which encodes interleukin-12 (“IL-12”) (e.g., the p35 and p40 subunits of IL-12) or a derivative thereof. In a specific embodiment the IL-12 transgene is an IL-12 transgene disclosed herein (see, e.g., Sections 5.2, 5.2.1, 5.7, 5.10, and 6). In a specific embodiment, the transgene comprises the nucleotide sequence set forth in SEQ ID NO: 26, 53, 61, 63, 66, or 68. In a specific embodiment, the transgene encodes a polypeptide, said polypeptide comprising the amino acid sequence set forth in SEQ ID NO: 22, 39, 42, or 43. In a specific embodiment, the IL-12 is human IL-12. In a specific embodiment, the IL-12 or derivative thereof is expressed by cells infected with the chimeric NDV. In a specific embodiment, the chimeric NDV can be used for the treatment of, e.g., cancer. A chimeric NDV disclosed herein unexpectedly significantly increases the Gene Expression Profiling Score of the 18-gene signature in Table 15 (see Section 6.3.1.14, infra) for tumor samples.

In another aspect, presented herein are methods for treating cancer utilizing a chimeric NDV or a composition comprising such a chimeric NDV in combination with an antagonist of PD-1 (e.g., human PD-1) or a ligand thereof or composition comprising such an antagonist, wherein the chimeric NDV comprises a packaged genome which encodes IL-12 (e.g., the p35 and p40 subunits of IL-12) or a derivative thereof. In a specific embodiment, the IL-12 or derivative thereof is expressed by cells infected with the chimeric NDV.

The methods of treating cancer described herein are based, in part, on the robust antitumor activity seen in subjects intratumorally administered a chimeric NDV comprising a packaged genome engineered to encode an IL-12 transgene (“NDV-IL-12”), in combination with the administration of an anti-PD-1 antibody that blocks the interaction between PD-1 and PD-L1. The robust antitumor activity in the treated subjects was observed in both the NDV-IL-12-injected and non-injected tumor, hence, an abscopal effect was observed. The methods of treating cancer described herein are also based, in part, on gene expression observed in subjects administered NDV-IL-12, alone or in combination with an anti-PD-1 antibody that blocks the interaction between PD-1 and a ligand thereof (e.g., PD-L1, PD-L2 or both PD-L1 and PD-L2).

In one embodiment, presented herein are methods for treating cancer comprising administering to a subject a chimeric NDV and an antagonist of PD-1 (e.g., human PD-1) or a ligand thereof, wherein the chimeric NDV comprises a packaged genome comprising a transgene encoding IL-12 or a derivative thereof. In a specific embodiment, the IL-12 or derivative thereof is expressed by cells infected with the chimeric NDV. In another embodiment, presented herein are methods for treating cancer comprising administering to a subject an effective amount of a chimeric NDV and an effective amount of an antagonist of PD-1 (e.g., human PD-1) or a ligand thereof, wherein the chimeric NDV comprises a packaged genome comprising a transgene encoding IL-12 or a derivative thereof. In a specific embodiment, the IL-12 or derivative thereof is expressed by cells infected with the chimeric NDV. The chimeric NDV and antagonist may be administered concurrently or sequentially to the subject. In certain embodiments, the chimeric NDV and antagonist are administered in the same composition. In other embodiments, the chimeric NDV and antagonist are administered in different compositions. The NDV and the antagonist may be administered by the same or different routes of administration to the subject. In a specific embodiment, the chimeric NDV is administered to a subject intratumorally and the antagonist is administered to the subject intravenously.

In another embodiment, presented herein is a method for treating cancer, comprising administering to a subject in need thereof a first composition comprising a chimeric NDV and a second composition comprising an antagonist of PD-1 (e.g., human PD-1) or a ligand thereof, wherein the chimeric NDV comprises a packaged genome comprising a transgene encoding IL-12, wherein the transgene encodes an IL-12 p40 subunit and an IL-12 p35 subunit. In another embodiment, presented herein is a method for treating cancer, comprising administering to a subject in need thereof a chimeric NDV and an antagonist of PD-1 (e.g., human PD-1) or a ligand thereof, wherein the chimeric NDV comprises a packaged genome which encodes IL-12, and wherein the antagonist of PD-1 is an anti-PD-1 antibody that blocks the interaction between PD-1 and a ligand thereof (e.g., PD-L1, PD-L2 or both PD-L1 and PD-L2). In another embodiment, presented herein is a method for treating cancer, comprising administering to a subject in need thereof a first composition comprising a chimeric NDV and a second composition comprising an anti-PD-1 antibody that blocks the interaction between PD-1 and a ligand thereof (e.g., PD-L1, PD-L2 or both PD-L1 and PD-L2), wherein the chimeric NDV comprises a packaged genome comprising a transgene encoding IL-12 (e.g., human IL-12), wherein the transgene encodes an IL-12 p40 subunit and an IL-12 p35 subunit. In a specific embodiment, presented herein is a method for treating cancer, comprising administering to a human subject in need thereof a first composition comprising a chimeric NDV and a second composition comprising an anti-PD-1 antibody that blocks the interaction between PD-1 and a ligand thereof (e.g., PD-L1, PD-L2 or both PD-L1 and PD-L2), wherein the chimeric NDV comprises a packaged genome comprising a transgene encoding human IL-12, wherein the transgene encodes a human IL-12 p40 subunit and a human IL-12 p35 subunit. The first and second compositions may be administered by same or different routes of administration. In a specific embodiment, the first composition is administered intratumorally and the second composition is administered intravenously. See, e.g., Sections 5.1, 5.2 and 6, infra for information regarding NDV, Sections 5.5 and 6, infra, for information regarding antagonists of PD-1 or a ligand thereof (including PD-1 blocking antibodies), Section 5.5.1, infra, for information regarding compositions and routes of administration, and Sections 5.7 and 6, infra, for information regarding methods for treating cancer.

In another aspect, provided herein is a chimeric NDV for use in a method of treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit, and wherein the method further comprises administering an antagonist of PD-1 (e.g., human PD-1) or a ligand thereof. In another embodiment, presented herein is a chimeric NDV for use in a method of treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit, wherein the method further comprises administering an antagonist of PD-1 or a ligand thereof, and wherein the antagonist of PD-1 (e.g., human PD-1) is an anti-PD-1 antibody that blocks the interaction between PD-1 and a ligand thereof (e.g., PD-L1, PD-L2 or both PD-L1 and PD-L2). In another embodiment, presented herein is a chimeric NDV for use in a method of treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit, and wherein the method further comprises administering an anti-PD-1 antibody that blocks the interaction between PD-1 and a ligand thereof (e.g., PD-L1, PD-L2 or both PD-L1 and PD-L2). The chimeric NDV and the antagonist of PD-1 or a ligand thereof or the anti-PD-1 antibody may be administered by same or different routes of administration. In a specific embodiment, the chimeric NDV is administered intratumorally and the antagonist of PD-1 or a ligand thereof or the anti-PD-1 antibody is administered intravenously. See, e.g., Sections 5.1, 5.2 and 6, infra for information regarding NDV, Sections 5.5 and 6, infra, for information regarding antagonists of PD-1 or a ligand thereof (including PD-1 blocking antibodies), Section 5.5.1, infra, for information regarding compositions and routes of administration, and Sections 5.7 and 6, infra, for information regarding methods for treating cancer.

In another embodiment, presented herein is a use of a chimeric NDV in the preparation of a medicament for use in combination with an antagonist of PD-1 (e.g., human PD-1) or a ligand thereof for treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit. In another embodiment, presented herein is a use of a chimeric NDV in the preparation of a medicament for use in combination with an antagonist of PD-1 (e.g., human PD-1) or a ligand thereof for treating cancer in a subject (e.g., a human subject), wherein the chimeric NDV comprises a packaged genome which encodes IL-12, and wherein the antagonist of PD-1 is an anti-PD-1 antibody that blocks the interaction between PD-1 and a ligand thereof (e.g., PD-L1, PD-L2 or both PD-L1 and PD-L2). In another embodiment, presented herein is a use of a chimeric NDV in the preparation of a medicament for use in combination with an anti-PD-1 antibody that blocks the interaction between PD-1 and a ligand thereof (e.g., PD-L1, PD-L2 or both PD-L1 and PD-L2) for treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit. The chimeric NDV and the antagonist of PD-1 or a ligand thereof or the anti-PD-1 antibody may be administered by same or different routes of administration. In a specific embodiment, the chimeric NDV is administered intratumorally and the and the antagonist of PD-1 or a ligand thereof or the anti-PD-1 antibody is administered intravenously. See, e.g., Sections 5.1, 5.2 and 6, infra for information regarding NDV, Sections 5.5 and 6, infra, for information regarding antagonists of PD-1 or a ligand thereof (including PD-1 blocking antibodies), Section 5.5.1, infra, for information regarding compositions and routes of administration, and Sections 5.7 and 6, infra, for information regarding methods for treating cancer.

The chimeric NDV may have the backbone of any NDV type or strain, including, but not limited to, naturally-occurring strains, variants or mutants, mutagenized viruses, reassortants and/or genetically engineered viruses. In a specific embodiment, the NDV that serves as the backbone for genetic engineering of the chimeric NDV is a lentogenic strain. In a specific embodiment, the NDV that serves as the backbone for genetic engineering of the chimeric NDV is a mesogenic strain. In a specific embodiment, the NDV that serves as the backbone for genetic engineering of the chimeric NDV is a velogenic strain. In one embodiment, the chimeric NDV comprises a packaged genome comprising a nucleotide sequence encoding a mutated F protein with a mutated cleavage site. In a specific embodiment, the chimeric NDV comprises a packaged genome comprising a nucleotide sequence encoding a mutated F protein in which the cleavage site of the F protein is mutated to produce a polybasic amino acid sequence, which allows the protein to be cleaved by intracellular proteases, which makes the virus more effective in entering cells and forming syncytia. In another specific embodiment, the chimeric NDV comprises a packaged genome comprising a nucleotide sequence encoding a mutated F protein in which the cleavage site of the F protein is replaced with one containing one or two extra arginine residues, allowing the mutant cleavage site to be activated by ubiquitously expressed proteases of the furin family. Specific examples of NDVs that express such a mutated F protein include, but are not limited to, rNDV/F2aa and rNDV/F3aa. For a description of mutations introduced into a NDV F protein to produce a mutated F protein with a mutated cleavage site, see, e.g., Park et al. (2006) Engineered viral vaccine constructs with dual specificity: avian influenza and Newcastle disease. PNAS USA 103: 8203-2808, which is incorporated herein by reference in its entirety.

In certain embodiments, the chimeric NDV comprises a mutated F protein with an F protein cleavage site of NDV LaSota strain or glycoprotein B of cytomegalovirus (CMV). In a specific embodiment, the chimeric NDV comprises a mutated F protein with an F protein cleavage site derived from glycoprotein B of cytomegalovirus, said F protein cleavage site comprising the amino acid sequence ¹¹¹H-N-R-T-K-S/F¹¹⁷ (SEQ ID NO: 56), such as described in International Patent Application Publication No. WO 2015/032755. In a specific embodiment, the chimeric NDV comprises a mutated F protein with an F protein cleavage site having one of the following sequences: S116: ¹¹¹H-N-R-T-K-S/F¹¹⁷ (SEQ ID NO: 56); S116K: ¹¹¹H-N-K-T-K-S/F¹¹⁷ (SEQ ID NO: 58); S116m: ¹¹¹H-N-R-M-K-S/F¹¹⁷ (SEQ ID NO: 69); S116KM: 11H-N-K-M-S/F-I¹¹⁸ (SEQ ID NO: 70); or R116: ¹¹¹H-N-R-T-K-R/F-I¹¹⁸ (SEQ ID NO: 71), such as described in International Patent Application No. WO 2015/032755. See, e.g., International Patent Application Publication No. WO 2015/032755 for a description of the types of mutated F protein cleavage sites that may be engineered into an NDV F protein, which is incorporated herein by reference in its entirety. In some embodiments, the chimeric NDV comprises a packaged genome comprising a nucleotide sequence encoding a mutated F protein, wherein the mutated F protein comprises the amino acid mutation L289A (i.e., an L to A mutation at the amino acid position corresponding to L289 of the LaSota F protein). In specific embodiments, the L289A mutated F protein possesses one, two or three arginine residues in the cleavage site. In certain embodiments, the mutated F protein is from a different type or strain of NDV than the backbone NDV. In other embodiments, the mutated F protein is from the same type or strain of NDV as the backbone NDV. In some embodiments, the mutated F protein is in addition to the backbone NDV F protein. In specific embodiments, the mutated F protein replaces the backbone NDV F protein.

The antagonist of PD-1 or a ligand thereof for use in combination with an NDV described herein (see, e.g., Section 5.1, Section 5.2, Section 5.5, Section 5.7, and/or Section 6) may be any antagonist of PD-1 or a ligand thereof known to one of skill in the art. Specific examples of ligands for PD-1 include PD-L1 and PD-L2. In a specific embodiment, the antagonist of PD-1 or a ligand thereof blocks (completely or partially) the interaction between a native ligand of PD-1 (e.g., PD-L1, PD-L2 or both PD-L1 and PD-L2) and PD-1. In a preferred embodiment, the antagonist of PD-1 or a ligand thereof blocks (completely or partially) the interaction between a native ligand of PD-1 (e.g., PD-L1, PD-L2 or both PD-L1 and PD-L2) and PD-1, and prevents or reduces the transduction of an inhibitory signal(s) induced by the interaction of PD-1 and native ligand of PD-1 (e.g., PD-L1 and/or PD-L2).

In a particular aspect, an antagonist of PD-1 or a ligand thereof is an antagonist of PD-1. In a specific embodiment, the antagonist of PD-1 or a ligand thereof is an antibody that binds to PD-1. In a specific embodiments, the antagonist of PD-1 or a ligand thereof is an antibody that binds to PD-1 and blocks (completely or partially) the interaction between PD-1 and PD-L1. In a specific embodiments, the antagonist of PD-1 or a ligand thereof is an antibody that binds to PD-1, blocks (completely or partially) the interaction between PD-1 and PD-L1, and prevents or reduces the transduction of an inhibitory signal(s). In a preferred embodiment, the antagonist of PD-1 or a ligand thereof is an antibody that binds to PD-1 and blocks (completely or partially) the interaction between PD-1 and its ligands, PD-L1 and PD-L2. In another preferred embodiment, the antagonist of PD-1 or a ligand thereof is an antibody that binds to PD-1, blocks the interaction between PD-1 and its ligands, PD-L1 and PD-L2, and prevents or reduces the transduction of an inhibitory signal(s). In certain embodiments, the antibody is a camelized antibody, human antibody or humanized antibody. In a specific embodiment, the antibody is a monoclonal antibody. In another specific embodiment, the antibody is a scFv.

In another aspect, an antagonist of PD-1 or a ligand thereof is an antagonist of a ligand of PD-1 (e.g., PD-L1 or PD-L2). In a specific embodiment, an antagonist of PD-1 or a ligand thereof is an antagonist of PD-L1. In certain embodiments, an antagonist of PD-1 or a ligand thereof is an antagonist of PD-L1, which binds to PD-L1 and blocks (completely or partially) the interaction between PD-1 to PD-L1. In some embodiments, an antagonist of PD-1 or a ligand thereof is an antagonist of PD-L1, which binds to PD-L1, blocks (completely or partially) the interaction between PD-1 to PD-L1 and prevents or reduces the transduction of an inhibitory signal(s). In some embodiments, an antagonist of PD-L1 also blocks (completely or partially) the interaction between PD-L1 and CD80 (B7.1).

In a specific embodiment, an antagonist of PD-1 or a ligand thereof is an antagonist of PD-L2. In certain embodiments, an antagonist of PD-1 or a ligand thereof is an antagonist of PD-L2, which binds to PD-L2 and blocks (completely or partially) the interaction between PD-1 to PD-L2. In certain embodiments, an antagonist of PD-1 or a ligand thereof is an antagonist of PD-L2, which binds to PD-L2 and blocks (completely or partially) the interaction between PD-1 to PD-L2 and prevents or reduces the transduction of an inhibitory signal(s).

In a specific embodiment, the antagonist of PD-1 or a ligand thereof is an antibody or a soluble receptor that specifically binds to a native ligand of PD-1. In a specific embodiment, the antagonist of PD-1 or a ligand thereof is an antibody or a soluble receptor that specifically binds to a native ligand of PD-1 and blocks (completely or partially) the native ligand from binding PD-1 and transducing an inhibitory signal(s). In certain specific embodiments, the soluble receptor is a fragment of PD-1 or a fragment of a derivative of PD-1 that binds to a ligand of PD-1 (e.g., the extracellular domain of PD-1 or a derivative of PD-1). In some specific embodiments, the soluble receptor is a fusion protein comprising at least a portion of PD-1 or a derivative of PD-1 (e.g., the extracellular domain of PD-1 or a derivative of PD-1), and a heterologous amino acid sequence. In a specific embodiment, the fusion protein comprises at least a portion of PD-1 or a derivative of PD-1, and the Fc portion of an immunoglobulin or a fragment thereof. In a specific embodiment, the portion of the PD-1 or a derivaitive thereof is e.g., the extracellular domain of PD-1 or a derivative thereof.

In a specific embodiment, the antagonist of PD-1 or a ligand thereof is an antibody that binds to a native ligand of PD-1. The antibody may bind to PD-L1 or PD-L2. In certain embodiments, the antibody is camelized antibody, human antibody or humanized antibody. In a specific embodiment, the antibody may be a monoclonal antibody. In another specific embodiment, the antibody may be a single-chain variable fragment (scFv). In a specific embodiment, the antagonist of PD-1 or a ligand thereof is an antibody or ligand that binds to PD-1, but does not transduce an inhibitory signal(s). In another embodiment, the antagonist of PD-1 or a ligand thereof is a ligand that binds to PD-1, but does not transduce an inhibitory signal(s). In certain specific embodiments, the ligand is a fusion protein comprising at least a portion of a ligand of PD-1 or a derivative of a ligand of PD-1, and a heterologous amino acid sequence. In specific embodiments, the fusion protein comprises at least a portion of a ligand of PD-1 or a derivative of a ligand of PD-1, and the Fc portion of an immunoglobulin or a fragment thereof.

In a specific embodiment, an antagonist of PD-1 or a ligand thereof is a PD-1 blocking antibody. In another specific embodiment, an antagonist of PD-1 or a ligand thereof is nivolumab. In a preferred embodiment, an antagonist of PD-1 or a ligand thereof is pembrolizumab. In another specific embodiment, an antagonist of PD-1 or a ligand thereof is a PD-L1 blocking antibody (e.g., duralumab or avelumab).

In one aspect, provided herein is a method for treating cancer, comprising administering to a human subject in need thereof a first composition comprising a chimeric Newcastle disease virus (NDV) and a second composition comprising an antagonist of human PD-1 or a ligand thereof, wherein the chimeric NDV comprises a packaged genome comprising a transgene encoding human interleukin-12 (“IL-12”), wherein the transgene encodes a human IL-12 p40 subunit and a human IL-12 p35 subunit. In a specific embodiment, the packaged genome of the chimeric NDV further comprises a nucleotide sequence encoding a mutated F protein and the mutated F protein is incorporated into the virion of the chimeric NDV, wherein the mutated F protein comprises a mutated cleavage site. In a specific embodiment, the mutated cleavage site is ¹¹¹H-N-R-T-K-R/F-I¹¹⁸ (SEQ ID NO: 71). In another specific embodiment, the packaged genome of the chimeric NDV further comprises a nucleotide sequence encoding a mutated F protein with the amino acid mutation L289A, wherein the mutated F protein is incorporated into the virion of the chimeric NDV. In a specific embodiment, the transgene comprises the nucleotide sequence set forth in SEQ ID NO:26. In another specific embodiment, the IL-12 p40 subunit comprises the amino acid sequence set forth in SEQ ID NO: 38. In another specific embodiment, the IL-12 p40 subunit comprises the amino acid sequence set forth in SEQ ID NO: 40. In another specific embodiment, the IL-12 p40 subunit comprises the amino acid sequence set forth in SEQ ID NO: 23. In another specific embodiment, the IL-12 p35 subunit comprises the amino acid sequence set forth in SEQ ID NO: 41. In another specific embodiment, the IL-12 p35 subunit comprises the amino acid sequence set forth in SEQ ID NO: 25. In another specific embodiment, the IL-12 p40 subunit comprises the amino acid sequence set forth in SEQ ID NO: 38 and the IL-12 p35 subunit comprises the amino acid sequence set forth in SEQ ID NO: 41. In another specific embodiment, the IL-12 p40 subunit comprises the amino acid sequence set forth in SEQ ID NO: 38 and the IL-12 p35 subunit comprises the amino acid sequence set forth in SEQ ID NO: 25. In a specific embodiment, the transgene encodes an amino acid sequence set forth in SEQ ID NO: 42. In a specific embodiment, the transgene encodes an amino acid sequence set forth in SEQ ID NO: 22. In a specific embodiment, the transgene encodes an amino acid sequence comprising the amino acid sequence set forth in SEQ ID NO: 43. In a specific embodiment, the transgene encodes an amino acid sequence comprising the amino acid sequence set forth in SEQ ID NO: 39. In another specific embodiment, the IL-12 p40 subunit comprises the amino acid sequence set forth in SEQ ID NO: 40 and the IL-12 p35 subunit comprises the amino acid sequence set forth in SEQ ID NO: 41. In a specific embodiment, the transgene is inserted between two transcription units of the packaged genome. In a particular embodiment, the two transcription units of the packaged genome are the transcription units for the NDV P gene and the NDV M gene. In a specific embodiment, the first composition is administered to the subject intratumorally or intra-nodally. In another specific embodiment, the second composition is administered to the subject intravenously.

In another aspect, provided herein is a chimeric NDV for use in a method for treating cancer in a human subject, wherein the chimeric NDV comprises a packaged genome comprising a transgene encoding human interleukin-12 (“IL-12”), wherein the transgene encodes a human IL-12 p40 subunit and a human IL-12 p35 subunit, and wherein the method further comprises administering an antagonist of human PD-1 or a ligand thereof. In a specific embodiment, the packaged genome of the chimeric NDV further comprises a nucleotide sequence encoding a mutated F protein and the mutated F protein is incorporated into the virion of the chimeric NDV, wherein the mutated F protein comprises a mutated cleavage site. In a specific embodiment, the mutated cleavage site is ¹¹¹H-N-R-T-K-R/F-I¹¹⁸ (SEQ ID NO: 71). In another specific embodiment, the packaged genome of the chimeric NDV further comprises a nucleotide sequence encoding a mutated F protein with the amino acid mutation L289A, wherein the mutated F protein is incorporated into the virion of the chimeric NDV. In a specific embodiment, the transgene comprises the nucleotide sequence set forth in SEQ ID NO:26. In another specific embodiment, the IL-12 p40 subunit comprises the amino acid sequence set forth in SEQ ID NO: 38. In another specific embodiment, the IL-12 p40 subunit comprises the amino acid sequence set forth in SEQ ID NO: 23. In another specific embodiment, the IL-12 p35 subunit comprises the amino acid sequence set forth in SEQ ID NO: 25. In another specific embodiment, the IL-12 p40 subunit comprises the amino acid sequence set forth in SEQ ID NO: 38 and the IL-12 p35 subunit comprises the amino acid sequence set forth in SEQ ID NO: 25. In a specific embodiment, the transgene encodes an amino acid sequence set forth in SEQ ID NO: 22. In a specific embodiment, the transgene encodes an amino acid sequence comprising the amino acid sequence set forth in SEQ ID NO: 39. In another specific embodiment, the IL-12 p35 subunit comprises the amino acid sequence set forth in SEQ ID NO: 41. In another specific embodiment, the IL-12 p40 subunit comprises the amino acid sequence set forth in SEQ ID NO: 38 and the IL-12 p35 subunit comprises the amino acid sequence set forth in SEQ ID NO: 41. In a specific embodiment, the transgene encodes an amino acid sequence set forth in SEQ ID NO: 42. In another specific embodiment, the IL-12 p40 subunit comprises the amino acid sequence set forth in SEQ ID NO: 40 and the IL-12 p35 subunit comprises the amino acid sequence set forth in SEQ ID NO: 41. In a specific embodiment, the transgene is inserted between two transcription units of the packaged genome. In a particular embodiment, the two transcription units of the packaged genome are the transcription units for the NDV P gene and the NDV M gene. In a specific embodiment, the sequence of the packaged genome is as set forth in SEQ ID NO: 51. In a specific embodiment, the sequence of the packaged genome is as set forth in SEQ ID NO: 52. In a specific embodiment, the first composition is administered to the subject intratumorally or intra-nodally. In another specific embodiment, the second composition is administered to the subject intravenously.

In a specific embodiment, the chimeric NDV comprises an NDV backbone which is lentogenic. In another specific embodiment, the chimeric NDV comprises an NDV backbone of LaSota strain. In another specific embodiment, the chimeric NDV comprises an NDV backbone of Hitchner B1 strain. In another specific embodiment, the chimeric NDV comprises an NDV backbone of a r73T-R116 virus.

In a specific embodiment, the antagonist of human PD-1 or a ligand thereof is an antibody that binds to human PD-1. In another specific embodiment, the antagonist of human PD-1 or a ligand thereof is an antibody that binds to human PD-1 and blocks the interaction between human PD-1 and its ligands, PD-L1 and PD-1.2. In a preferred embodiment, the antibody is pembrolizumab. In another embodiment, the antibody is nivolumab or MEDI0680. In a specific embodiment, the antibody comprises a variable light chain region (VLCR) complementarity determining region (CDR) 1 comprising the amino acid sequence RASKGVSTSGYSYLH (SEQ ID NO: 1), a VLCR CDR2 comprising the amino acid sequence LASYLES (SEQ ID NO: 2), a VLCR CDR3 comprising the amino acid sequence QHSRDLPLT (SEQ ID NO: 3), a variable heavy chain region (VHCR) CDR 1 comprising the amino acid sequence NYYMY (SEQ ID NO: 6), a VHCR CDR2 comprising the amino acid sequence GINPSNGGTNFNEKFKN (SEQ ID NO: 7), and a VHCR CDR3 comprising the amino acid sequence RDYRFDMGFDY (SEQ ID NO: 8). In another embodiment, the antibody comprises: (a) a VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a VHCR comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSS (SEQ ID NO: 9). In another embodiment, the antibody comprises: (a) a light chain comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIKRTVAAPSVFI FPPSDEQLKSGTASVVCLLNNFYPREAKVQWKVDNALQSGNSQESVTEQDSKDSTYSLS STLTLSKADYEKHKVYACEVTHQGLSSPVTKSFNRGEC (SEQ ID NO: 5); and (b) a heavy chain comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPCSRSTSESTAALGCLVKDYFPEPVTVSWNSGALTSGVHTF PAVLQSSGLYSLSSVVTVPSSSLGTKTYTCNVDHKPSNTKVDKRVESKYGPPCPPCPAPE FLGGPSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLT VDKSRWQEGNVFSCSVMHEALHINHYTQKSLSLSLGK (SEQ ID NO: 10). In another embodiment, the antibody comprises a VLCR CDR1 comprising the amino acid sequence RASQSVSSYLA (SEQ ID NO: 11), a VLCR CDR2 comprising the amino acid sequence DASNRAT (SEQ ID NO: 12), a VLCR CDR3 comprising the amino acid sequence QQSSNWPRT (SEQ ID NO: 13), a VHCR CDR1 comprising the amino acid sequence NSGMH (SEQ ID NO: 16), a VHCR CDR2 comprising the amino acid sequence VIWYDGSKRYYADSVKG (SEQ ID NO: 17), and a VHCR CDR3 comprising the amino acid sequence NDDY (SEQ ID NO: 18). In another embodiment, the antibody comprises:
(a) a VLCR comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIK (SEQ ID NO: 14);
and (b) a VHCR comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSS (SEQ ID NO: 19). In another embodiment, the antibody comprises: (a) a light chain comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIKRTVAAPSVFIFPPS DEQLKSGTASVVCLLNNFYPREAKVQWKVDNALQSGNSQESVTEQDSKDSTYSLSSTL TLSKADYEKHKVYACEVTHQGLSSPVTKSFNRGEC (SEQ ID NO: 15); and (b) a heavy chain comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSSAS TKGPSVFPLAPCSRSTSESTAALGCLVKDYFPEPVTVSWNSGALTSGVHTFPAVLQSSGL YSLSSVVTVPSSSLGTKTYTCNVDHKPSNTKVDKRVESKYGPPCPPCPAPEFLGGPSVFL FPPKPKDTLMISRTPEVTCVVVDVSQEDPEVQFNWYVDGVEVHNAKTKPREEQFNSTY RVVSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFFLYSRLTVDKSRWQ EGNVFSCSVMHEALHNHYTQKSLSLSLGK (SEQ ID NO: 20).

In a specific embodiment, the antagonist of human PD-1 or a ligand thereof is an antibody that binds to human PD-L1. In a particular embodiment, the antibody is durvalumab, avelumab, bms-936559, or atezolizumab.

In a specific embodiment, the administration of a chimeric NDV described herein in a method of treating cancer provided herein induces IL-12p70, IFN-γ expression, or both IL-12p70 and IFN-γ expression. In a specific embodiment, the administration of a chimeric NDV described herein in a method of treating cancer provided herein increases the gene expression profile (GEP) score. See, for example, Example 6.3, infra, regarding the GEP score. In a specific embodiment, provided herein is a method of increasing the response to anti-PD-1 therapy.

In a specific embodiment, the patient treated in accordance with the methods disclosed herein exhibits cutaneous or subcutaneous tumors or tumors within the lymph node.

In another specific embodiment, the cancer is melanoma, kidney cancer, lung cancer (e.g., non-small cell lung cancer), bladder cancer, ovarian cancer, colon cancer, pancreatic cancer, renal cancer (e.g., renal cell carcinoma), colorectal cancer (e.g., colorectal carcinoma), breast cancer (e.g., breast carcinoma), or head and neck cancer (e.g., squamous cell carcinoma of the head and neck). In a particular embodiment, the cancer is a solid tumor selected from the group consisting of melanoma, head and neck squamous cell carcinoma, and breast carcinoma. In another specific embodiment, the cancer is non-Hodgkin lymphoma or Hodgkin lymphoma. In another specific embodiment, the cancer is metastatic. In another specific embodiment, the cancer is unresectable. In particular embodiments, the cancer comprises a dermal, subcutaneous, or nodal metastasis. In a specific embodiment, the cancer is refractory or relapsed, or both. In a specific embodiment, a biopsy of the cancer is PD-L1-positive. In a particular embodiment, the biopsy has a tumor proportion score of at least 1%. In other specific embodiments, a biopsy of the cancer is PD-L1-negative. In a particular embodiment, the biopsy has a tumor proportion score of less than 1%.

In a specific embodiment, the subject is refractory to monotherapy treatment with an antibody that binds to PD-1 and blocks the interaction between PD-1 and its ligands, PD-L1 and PD-L2. In another specific embodiment, the subject is refractory or unresponsive to monotherapy treatment with an antagonist of human PD-1 or a ligand thereof. In a particular embodiment, the subject is refractory or unresponsive to monotherapy with nivolumab, AMP-224, MEDI0680, pembrolizumab, durvalumab, avelumab, bms-936559, or atezolizumab.

### 3.1 Terminology

As used herein, the term “about” or “approximately” when used in conjunction with a number refers to any number within 1, 5 or 10% of the referenced number.

As used herein, the term “agonist(s)” refers to a molecule(s) that binds to another molecule and induces a biological reaction. In a specific embodiment, an agonist is a molecule that binds to a receptor on a cell and triggers one or more signal transduction pathways. For example, an agonist includes an antibody or ligand that binds to a receptor on a cell and induces one or more signal transduction pathways. In certain embodiments, the antibody or ligand binds to a receptor on a cell and induces one or more signal transduction pathways. In other embodiments, the agonist facilitates the interaction of the native ligand with the native receptor.

The term “amino acid sequence identity” refers to the degree of identity or similarity between a pair of aligned amino acid sequences, usually expressed as a percentage. Percent identity is the percentage of amino acid residues in a candidate sequence that are identical (i.e., the amino acid residues at a given position in the alignment are the same residue) or similar (i.e., the amino acid substitution at a given position in the alignment is a conservative substitution, as discussed below), to the corresponding amino acid residue in the peptide (or polypeptide or protein) after aligning the sequences and introducing gaps, if necessary, to achieve the maximum percent sequence homology.

As used herein, the term “antagonist(s)” refers to a molecule(s) that inhibits the action of another molecule without provoking a biological response itself. In a specific embodiment, an antagonist is a molecule that binds to a receptor on a cell and blocks or dampens the biological activity of an agonist. For example, an antagonist includes an antibody or ligand that binds to a receptor on a cell and blocks or dampens binding of the native ligand to the receptor without inducing one or more signal transduction pathways. Another example of an antagonist includes an antibody or soluble receptor that competes with the native receptor on cells for binding to the native ligand, and thus, blocks or dampens one or more signal transduction pathways induced when the native receptor binds to the native ligand. Another example of an antagonist includes an antibody or soluble receptor that does not prevent the binding of the native receptor with the native ligand, but prevents signal transduction by other means (e.g., through inhibition of receptor multimerization).

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass. In a specific embodiment, an antibody is a human or humanized antibody. In another specific embodiment, an antibody is a monoclonal antibody or scFv. In certain embodiments, an antibody is a human or humanized monoclonal antibody or scFv. In other specific embodiments, the antibody is a bispecific antibody. In certain embodiments, the bispecific antibody specifically binds to a co-stimulatory receptor of an immune cell or an inhibitory receptor of an immune cell, and a receptor on a cancer cell. In some embodiments, the bispecific antibody specifically binds to two receptors on immune cells, e.g., two co-stimulatory receptors on immune cells, two inhibitory receptors on immune cells, or one co-stimulatory receptor on immune cells and one inhibitory receptor on immune cells.

“Conservative substitution” is a term understood by those of skill in the art and generally refers to replacement of an amino acid of one class with another amino acid of the same class. In particular embodiments, a conservative substitution does not alter the structure or function, or both, of a polypeptide. Classes of amino acids for the purposes of conservative substitution may include hydrophobic, neutral hydrophilic, acidic, basic, conformation disrupters, and aromatic. Hydrophobic amino acids may include Met, Ala, Val, Leu, and Ile. Neutral hydrophilic amino acids may include Cys, Ser, and Thr. Acidic amino acids may include Asp and Glu. Basic amino acids may include Asn, Gln, His, Lys, and Arg. Conformation disrupter amino acids may include Gly and Pro. Aromatic amino acids may include Trp, Tyr, and Phe.

As used herein, the term “derivative” in the context of proteins or polypeptides refers to: (a) a polypeptide that is at least 80%, 85%, 90%, 95%, 98%, or 99% or is 80% to 85%, 80% to 90%, 80% to 95%, 90% to 95%, 85% to 99%, or 95% to 99% identical to a native polypeptide; (b) a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical to a nucleic acid sequence encoding a native polypeptide; (c) a polypeptide that contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions and/or substitutions) relative to a native polypeptide; (d) a polypeptide encoded by nucleic acid sequence that can hybridize under high, moderate or typical stringency hybridization conditions to a nucleic acid sequence encoding a native polypeptide; (e) a polypeptide encoded by a nucleic acid sequence that can hybridize under high, moderate or typical stringency hybridization conditions to a nucleic acid sequence encoding a fragment of a native polypeptide of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids; or (f) a fragment of a native polypeptide. Derivatives also include a polypeptide that comprises the amino acid sequence of a naturally occurring mature form of a mammalian polypeptide and a heterologous signal peptide amino acid sequence. In addition, derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, derivatives include polypeptides comprising one or more non-classical amino acids. In one embodiment, a derivative is isolated. In specific embodiments, a derivative retains one or more functions of the native polypeptide from which it was derived.

Percent identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

As used herein, the term “fragment” is the context of a fragment of a proteinaceous agent (e.g., a protein) refers to a fragment that is 8 or more contiguous amino acids, 10 or more contiguous amino acids, 15 or more contiguous amino acids, 20 or more contiguous amino acids, 25 or more contiguous amino acids, 50 or more contiguous amino acids, 75 or more contiguous amino acids, 100 or more contiguous amino acids, 150 or more contiguous amino acids, 200 or more contiguous amino acids, or in the range of between 10 to 300 contiguous amino acids, 10 to 200 contiguous amino acids, 10 to 250 contiguous amino acids, 10 to 150 contiguous amino acids, 10 to 100 contiguous amino acids, 10 to 50 contiguous amino acids, 50 to 100 contiguous amino acids, 50 to 150 contiguous amino acids, 50 to 200 contiguous amino acids, 50 to 250 contiguous amino acids, 50 to 300 contiguous amino acids, 25 to 50 contiguous amino acids, 25 to 75 contiguous amino acids, 25 to 100 contiguous amino acids, or 75 to 100 contiguous amino acids of a proteinaceous agent. In a specific embodiment, a fragment of a proteinaccous agent retains one or more functions of the proteinaceous agent-in other words, it is a functional fragment. For example, a fragment of a proteinaceous agent retains the ability to interact with another protein and/or to induce, enhance or activate one or more signal transduction pathways.

As used herein, the term “functional fragment,” in the context of a proteinaceous agent, refers to a portion of a proteinaceous agent that retains one or more activities or functions of the proteinaceous agent. For example, a functional fragment of an inhibitory receptor may retain the ability to bind one or more of its ligands. A functional fragment of a ligand of a co-stimulatory receptor may retain the ability to bind to the receptor and/or induce, enhance or activate one or more signal transduction pathways mediated by the ligand binding to its co-stimulatory receptor.

As used herein, the term “GEP score” refers to RNA Gene Expressiong Profiling of human tumor histoculture samples based on the 18-gene signature in Table 15. Gene expression data of isolated RNA for each individual sample is normalized by HK (housekeeping) normalization. For each tumor sample, raw counts are log 10 transformed and then each gene normalized by subtracting off the arithmetic mean of all housekeeping genes (Table 15). Gene Expression Profiling (GEP) Signature scores are calculated as a weighted sum of the housekeeping normalized values of the 18 gene Up-Down Signature (Table 15). The house keeping normalized value for each gene is multiplied by the coefficient for that gene from the set of scoring weights to generate a weighted RNA value for each of the genes in the 18 gene signature, and adding the weighted RNA values to produce the signature score for the tumor sample.

As used herein, the term “heterologous” to refers an entity not found in nature to be associated with (e.g., encoded by and/or expressed by the genome of) a naturally occurring NDV.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1-year-old year human.

In certain embodiments, the term “highly fusogenic” and the like, as used herein, refers to an increase in the ability of the NDV to form syncytia involving a large number of cells. In a specific embodiment, cells infected with an NDV described herein that is engineered to express a mutated F protein have an increased ability to form syncytia relative to cells infected with the parental virus from which the virus is derived, which parental virus has an unmutated F protein. In another specific embodiment, about 10% to about 25%, about 25% to about 50%, about 25% to about 75%, about 50% to about 75%, about 50% to about 95%, or about 75% to about 99% or about 10%, 15%, 20%, 25%, 30%, 35%, 40%, 45%, 50%, 55%, 60%, 65%, 70%, 75%, 80%, 85%, 90%, 95%, or 99% more cells infected with an NDV described herein that is engineered to express a mutated F protein form syncytia relative to the number of cells forming syncytia that are infected with the parental virus from the chimeric virus is derived which has an unmutated F protein. In certain embodiments, the syncytia are quantitated microscopically by counting the number of nuclei per syncytium after a certain period of time (e.g., about 8 hours to about 12 hours, about 12 hours to about 24 hours, about 24 hours to about 36 hours, or about 36 hours to about 48 hours).

As used herein, the term “interferon antagonist” refers to an agent that reduces or inhibits the cellular interferon immune response. In one embodiment, an interferon antagonist is a proteinaceous agent that reduces or inhibits the cellular interferon immune response. In a specific embodiment, an interferon antagonist is a viral protein or polypeptide that reduces or inhibits the cellular interferon response.

In a specific embodiment, an interferon antagonist is an agent that reduces or inhibits interferon expression and/or activity. In one embodiment, the interferon antagonist reduces or inhibits the expression and/or activity of type I IFN. In another embodiment, the interferon antagonist reduces or inhibits the expression and/or activity of type II IFN. In another embodiment, the interferon antagonist reduces or inhibits the expression and/or activity of type III IFN. In a specific embodiment, the interferon antagonist reduces or inhibits the expression and/or activity of either IFN-α, IFN-β or both. In another specific embodiment, the interferon antagonist reduces or inhibits the expression and/or activity of IFN-γ. In another embodiment, the interferon antagonist reduces or inhibits the expression and/or activity of one, two or all of IFN-α, IFN-β, and IFN-γ.

In certain embodiments, the expression and/or activity of IFN-α, IFN-β and/or IFN-γ in an embryonated egg or cell is reduced approximately 1 to approximately 100 fold, approximately 5 to approximately 80 fold, approximately 20 to approximately 80 fold, approximately 1 to approximately 10 fold, approximately 1 to approximately 5 fold, approximately 40 to approximately 80 fold, or 1, 2, 3, 4, 5, 7, 10, 15, 20, 25, 30, 35, 40, 45, 50, 55, 60, 65, 70, 75, 80, 85, 90, 95 or 100 fold by an interferon antagonist relative to the expression and/or activity of IFN-α, IFN-β, and/or IFN-γ in a control embryonated egg or a cell not expressing or not contacted with such an interferon antagonist as measured by the techniques described herein or known to one skilled in the art. In other embodiments, the expression and/or activity of IFN-α, IFN-β and/or IFN-γ in an embryonated egg or cell is reduced by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80% to 85%, at least 85% to 90%, at least 90% to 95%, at least 95% to 99% or by 20%, 25%, 30%, 35%, 40%, 45%, 50%, 55%, 60%, 65%, 70%, 75%, 80%, 85%, 90%, 95%, or 99% by an interferon antagonist relative to the expression and/or activity of IFN-α, IFN-β, and/or IFN-γ in a control embryonated egg or a cell not expressing or not contacted with such an interferon antagonist as measured by the techniques described herein or known to one skilled in the art.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, and/or are deficient in the activity of antiviral genes induced by one, two or more types of IFN, or induced by any type of IFN.

“Interleukin-12” and “IL-12” refer to any IL-12 known to those of skill in the art. In certain embodiments, the IL-12 may be human, dog, cat, horse, pig, or cow IL-12. In a specific embodiment, the IL-12 is human IL-12. A typical IL-12 consists of a heterodimer encoded by two separate genes, IL-12A (the p35 subunit) and IL-12B (the p40 subunit), known to those of skill in the art. GenBank™ accession number NM_000882.3 (GI number 325974478) provides an exemplary human IL-12A nucleic acid sequence. GenBank™ accession number NM_002187.2 (GI number 24497437) provides an exemplary human IL-12B nucleic acid sequence. GenBank™ accession number NP_000873.2 (GI number 24430219) provides an exemplary human IL-12A (the p35 subunit) amino acid sequence. GenBank™ accession number NP_002178.2 (GI number 24497438) provides an exemplary human IL-12B (the p40 subunit) amino acid sequence. In certain embodiments, an IL-12 consists of a single polypeptide chain, comprising the p35 subunit and the p40 subunit, optionally separated by a linker sequence (such as, e.g., any one of SEQ ID NOs: 24 and 46-49). In a preferred embodiment, an IL-12 comprises the p35 and p40 subunit sequences provided in Section 6, e.g., SEQ ID NOs: 25 and 23, respectively, or SEQ ID NOs: 41 and 40, respectively. In certain embodiments, an IL-12 consists of more than one polypeptide chain in quaternary association, e.g., p35 and p40. As used herein, the terms “interleukin-12” and “IL-12” encompass interleukin-12 polypeptides that are modified by post-translational processing such as signal peptide cleavage, disulfide bond formation, glycosylation (e.g., N-linked glycosylation), protease cleavage and lipid modification (e.g., S-palmitoylation). In some embodiments, one or both of the subunits of IL-12 or IL-12 consisting of a single polypeptide chain includes a signal sequence. In other embodiments, one or both of the subunits of IL-12 or IL-12 consisting of a single polypeptide chain does not include a signal sequence. The signal sequence can be the naturally occurring signal peptide sequence or a variant thereof. In some embodiments, the signal peptide is an IL-12 signal peptide. In some embodiments, the signal peptide is heterologous to an IL-12 signal peptide.

As used herein, the terms “immunospecifically binds,” “immunospecifically recognizes,” “specifically binds,” and “specifically recognizes” are analogous terms in the context of antibodies and refer to molecules that specifically bind to an antigen (e.g., epitope or immune complex) as understood by one skilled in the art. A molecule that specifically binds to an antigen may bind to other peptides or polypeptides with lower affinity as determined by, e.g., immunoassays (e.g., ELISA), surface plasmon resonance (e.g., BIAcore®), a KinEx assay (using, e.g., a KinExA 3000 instrument (Sapidyne Instruments, Boise, ID)), or other assays known in the art. In a specific embodiment, molecules that specifically bind to an antigen bind to the antigen with a dissociation constant (i.e., Ka) that is at least 2 logs, 2.5 logs, 3 logs, 3.5 logs, 4 logs or greater than the Ka when the molecules bind to another antigen.

As used herein, the term “monoclonal antibody” is a term of the art and generally refers to an antibody obtained from a population of homogenous or substantially homogeneous antibodies, and each monoclonal antibody will typically recognize a single epitope (e.g., single conformation epitope) on the antigen.

As used herein, the phrase “multiplicity of infection” or “MOI” is the average number of virus per infected cell. The MOI is determined by dividing the number of virus added (ml added×Pfu) by the number of cells added (ml added×cells/ml).

As used herein, the term “native ligand” refers to any naturally occurring ligand that binds to a naturally occurring receptor. In a specific embodiment, the ligand is a mammalian ligand. In another specific embodiment, the ligand is a human ligand.

As used herein, the term “native polypeptide(s)” in the context of proteins or polypeptides refers to any naturally occurring amino acid sequence, including immature or precursor and mature forms of a protein. In a specific embodiment, the native polypeptide is a human protein or polypeptide.

As used herein, the term “native receptor” refers to any naturally occurring receptor that binds to a naturally occurring ligand. In a specific embodiment, the receptor is a mammalian receptor. In another specific embodiment, the receptor is a human receptor.

“Programmed cell death protein 1”, “PD1”, and “PD-1” refer to any PD-1 known to those of skill in the art. In certain embodiments, the PD-1 may be human, dog, cat, horse, pig, or cow PD-1. In a specific embodiment, the PD-1 is human PD-1. GenBank™ accession number NM_005018.2 (GI number 167857791) provides an exemplary human PD-1 nucleic acid sequence. GenBank™ accession number NP_005009.2 (GI number 167857792) provides an exemplary human PD-1 amino acid sequence. Ligands of PD-1 include programmed death-ligand 1 (also referred to as “PD-L1”, “PDL1”, “cluster of differentiation 274”, “CD274”, “B7 homolog 1”, and “B7-H1”) and programmed cell death 1 ligand 2 (also referred to as “PDL2”, “PD-L2”, and “B7-DC”). PD-L1 refers to any PD-L1 known to those of skill in the art. In certain embodiments, the PD-L1 may be human, dog, cat, horse, pig, or cow PD-L1. In a specific embodiment, the PD-L1 is human PD-L1. GenBank™ accession numbers NM_001314029.1, NM_001267706.1, and NM_014143.3 (GI numbers 930425328, 390979638, and 292658763, respectively) provide exemplary human PD-L1 nucleic acid sequences. GenBank™ accession numbers NP_001300958.1, NP_001254635.1, and NP_054862.1 (GI numbers 930425329, 390979639, and 7661534, respectively) provide exemplary human PD-L1 amino acid sequences. PD-1.2 refers to any PD-L2 known to those of skill in the art. In certain embodiments, the PD-L2 may be human, dog, cat, horse, pig, or cow PD-L2. In a specific embodiment, the PD-L2 is human PD-L2. GenBank™ accession number NM_025239.3 (GI number 190014604) provides an exemplary human PD-1.2 nucleic acid sequence. GenBank™ accession number NP_079515.2 (GI number 190014605) provides an exemplary human PD-L2 amino acid sequence. As used herein, PD-1, PD-L1, and PD-L2 encompass PD-1, PD-L1 and PD-L2 polypeptides, respectively, that are modified by post-translational processing such as signal peptide cleavage, disulfide bond formation, glycosylation (e.g., N-linked glycosylation), protease cleavage and lipid modification (e.g., S-palmitoylation). In some embodiments, PD-1, PD-L1 and PD-L2 includes PD-1, PD-L1 and PD-L2 polypeptides, respectively, with a signal sequence. In other embodiments, PD-1, PD-L1 and PD-L2 includes PD-1, PD-L1 and PD-L2 polypeptides, respectively, that do not include a signal sequence. See, e.g., Ishida et al., 1992, EMBO J. 11:3887-3895 and Shinohara et al., 1994, Genomics 23:704-706 (each which is incorporated herein by reference in its entirety) for information regarding the structure of PD-1, including information regarding the signal sequence for PD-1. When used in the context of an antibody of PD-1 or ligand thereof, the antibody is directed to the mature form of PD-1, PD-L1 or PD-L2.

“Refractory” is an art recognized term which typically means a cancer that does not respond to treatment, i.e., does not receive a beneficial effect as described under “treatment”. The cancer may be resistant at the beginning of treatment or it may become resistant during treatment.

“Relapsed” is an art-recognized term, which typically means the return of a disease or the signs and symptoms of a disease after a period of improvement through treatment as described below.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, cow, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In other embodiments, the subject is a human. In certain embodiments, the mammal (e.g., human) is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the terms “treat”, “treatment” and “treating” in the context of the administration of a therapy refers to a treatment/therapy from which a subject may receive a beneficial effect, such as the reduction, decrease, attenuation, diminishment, stabilization, remission, suppression, inhibition or arrest of the development or progression of cancer, or a symptom thereof. In certain embodiments, the treatment/therapy that a subject receives results in at least one or more of the following effects: (i) the reduction or amelioration of the severity of cancer and/or a symptom associated therewith; (ii) the reduction in the duration of a symptom associated with cancer; (iii) the prevention in the recurrence of a symptom associated with cancer; (iv) the regression of cancer and/or a symptom associated therewith; (v) the reduction in hospitalization of a subject; (vi) the reduction in hospitalization length; (vii) the increase in the survival of a subject; (viii) the inhibition of the progression of cancer and/or a symptom associated therewith; (ix) the enhancement or improvement of the therapeutic effect of another therapy; (x) a reduction or elimination in the cancer cell population; (xi) a reduction in the growth of a tumor or neoplasm; (xii) a decrease in tumor size; (xiii) a reduction in the formation of a tumor; (xiv) eradication, removal, or control of primary, regional and/or metastatic cancer; (xv) a decrease in the number or size of metastases; (xvi) a reduction in mortality; (xvii) an increase in cancer-free survival rate of patients; (xviii) an increase in relapse-free survival; (xix) an increase in the number of patients in remission; (xx) a decrease in hospitalization rate; (xxi) the size of the tumor is maintained and does not increase in size or increases the size of the tumor by less than 5% or 10% after administration of a therapy as measured by conventional methods available to one of skill in the art, such as MRI, X-ray, CT Scan and PET scan; (xxii) the prevention of the development or onset of cancer and/or a symptom associated therewith; (xxiii) an increase in the length of remission in patients; (xxiv) the reduction in the number of symptoms associated with cancer; (xxv) an increase in symptom-free survival of cancer patients; (xxvi) limitation of or reduction in metastasis; (xxvii) overall survival; (xxviii) progression-free survival (as assessed, e.g., by RECIST v1.1.); (xxix) overall response rate; and/or (xxx) an increase in response duration. In some embodiments, the treatment/therapy that a subject receives does not cure cancer, but prevents the progression or worsening of the disease. In certain embodiments, the treatment/therapy that a subject receives does not prevent the onset/development of cancer, but may prevent the onset of cancer symptoms. Any method known to the skilled artisan may be utilized to evaluate the treatment/therapy that a subject receives. In a specific embodiment, the treatment/therapy is evaluated according to the Response Evaluation Criteria In Solid Tumors (“RECIST”) published rules. In a specific embodiment, the treatment/therapy is evaluated according to the RECIST rules published in February 2000 (also referred to as “RECIST 1”) (see, e.g., Therasse et al., 2000, Journal of National Cancer Institute, 92 (3): 205-216, which is incorporated by reference herein in its entirety). In a specific embodiment, the treatment/therapy is evaluated according to the RECIST rules published in January 2009 (also referred to as “RECIST 1.1”) (see, e.g., Eisenhauer et al., 2009, European Journal of Cancer, 45:228-247, which is incorporated by reference herein in its entirety). In a specific embodiment, the treatment/therapy is evaluated according to the RECIST rules utilized by the skilled artisan at the time of the evaluation. In a specific embodiment, the treatment/therapy is evaluated according to the immune related RECIST (“irRECIST”) published rules (see, e.g., Bohnsack et al., 2014, ESMO Abstract 4958, which is incorporated by reference herein in its entirety). In a specific embodiment, the treatment/therapy is evaluated according to the irRECIST rules utilized by the skilled artisan at the time of the evaluation. In a specific embodiment, the treatment/therapy is evaluated according to the Lugano criteria. In a specific embodiment, the treatment/therapy is evaluated through a reduction in tumor-associated serum markers, such as, e.g., CA-125, CEA, CA-19-9, PSA, AFP, inhibin A, inhibin B, HCG, CA 15-3, thyroglobulin, HE4.

As used herein, the term “in combination” in the context of the administration of (a) therapy (ies) to a subject, refers to the use of more than one therapy. The use of the term “in combination” does not restrict the order in which therapies are administered to a subject. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. For example, a chimeric NDV described herein may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an antagonist of PD-1 or a ligand thereof.

As used herein, the terms “therapies” and “therapy” can refer to any protocol(s), method(s), and/or agent(s) that can be used in the treatment of cancer. In certain embodiments, the terms “therapies” and “therapy” refer to biological therapy, supportive therapy, hormonal therapy, chemotherapy, immunotherapy and/or other therapies useful in the treatment of cancer. In a specific embodiment, a therapy includes adjuvant therapy. For example, using a therapy in conjunction with a drug therapy, biological therapy, surgery, and/or supportive therapy. In certain embodiments, the term “therapy” refers to a chimeric NDV described herein. In other embodiments, the term “therapy” refers to an agent that is not a chimeric NDV. In certain embodiments, the term “therapy” refers to an antagonist of PD-1 or a ligand thereof. In other embodiments, the term “therapy” refers to an agent that is not an antagonist of PD-1 or a ligand thereof. In certain embodiments, the term “therapy” refers to a chimeric NDV described herein and an antagonist of PD-1 or a ligand thereof. In certain embodiments, the term “therapy” refers to an agent that is neither a chimeric NDV described herein nor an antagonist of PD-1 or a ligand thereof.

### 4. BRIEF DESCRIPTION OF THE FIGURES

FIG. 1. NDV infection upregulates the expression of MHC I, MHC II, and ICAM-1 on the surface of in vitro infected B16F10 cells (24 hours post-infection).

FIGS. 2A-2E. Intratumoral NDV treatment leads to infiltration with macrophages, NK cells, CD8 and CD4 effector cells and decreases the frequency of Tregs. FIG. 2A) Overall study scheme. FIG. 2B) Total CD45+ infiltrates. FIG. 2C) Total immune cell infiltrates. FIG. 2D) Representative flow cytometry dot plots of relative CD4 FoxP3+ and FoxP3− subsets. FIG. 2F) Teff/Treg and CD8/Treg ratios.

FIGS. 3A-3C. Therapy with NDV exhibits favorable effects on tumor microenvironment of distant tumors. FIG. 3A) Representative flow cytometry dot plots of relative CD4 FoxP3+ and FoxP3− subsets. FIG. 3B) Absolute numbers of CD4 effector, Treg, and CD8 cells per gram of tumor. FIG. 3C) Teff/Treg and CD8/Treg ratios.

FIGS. 4A-4C. Lymphocytes infiltrating distant tumors upregulate activation, lytic, and proliferation markers. Representative expression plots on CD4 effector cells (top) and the corresponding percentages in the CD4 effector, CD8, Tregs (bottom) are shown for FIG. 4A) CD44, FIG. 4B) Granzyme B, and FIG. 4C) Ki-67.

FIGS. 5A-D. NDV Monotherapy delays the growth of distant tumors and provides some protection against tumor rechallenge. Bilateral flank tumors were established as described in FIG. 2A and the animals were treated and followed for survival. FIG. 5A) Growth of right flank (treated) tumors. FIG. 5B) Growth of left flank (non-treated) tumors. FIG. 5C) Overall survival. Numbers in boxes indicate percent of animals free of tumors. FIG. 5D) Survival in animals cured of B16F10 melanoma by NDV re-challenged on day 75 with B16F10 melanoma cells. Representative results of two different experiments with 10 mice per group.

FIGS. 5E-5F. Tumor-infiltrating lymphocytes from both treated and non-treated tumors upregulate CTLA-4 in response to NDV therapy. FIG. 5E) Representative dot plots of CTLA-4 expression in CD8, CD4 effector, and Tregs in right (treated) tumors. FIG. 5F) Representative dot plots of CTLA-4 expression in CD8, CD4 effector, and Tregs in left (non-treated) tumors.

FIGS. 6A-6C. NDV infection upregulates expression of PD-L1 in B16F10 tumors.

FIG. 6A) Surface PD-L1 expression on B16F10 cells infected with NDV for 24 hours. FIG. 6B) Surface PD-L1 expression on B16F10 cells treated with UV-inactivated supernatant from infected B16F10 cells. FIG. 6C) Upregulation of PD-L1 on the surface of tumor cells isolated from injected and distant tumors from the animals treated as in FIG. 2A (2 left panels-representative flow cytometry plots, right panel-calculated averages of 5 mice per group).

FIGS. 7A-7F. Combination therapy with NDV and anti-PD-1 is effective systemically against B16 melanoma and results in increased T cell infiltration with upregulation of activation markers. FIG. 7A) Overall survival. Animals were treated as described in FIG. 2A with or without anti-PD-1 antibody. FIG. 7B) Absolute numbers of CD45, CD3, CD8, and CD4 effector cells in tumors. FIG. 7C) Relative percentage of regulatory T cells in tumor-infiltrating lymphocytes. FIG. 7D and FIG. 7E) Tumor-infiltrating lymphocytes from distant tumors were isolated and stained for expression of ICOS (FIG. 7D) and Granzyme B (FIG. 7E). FIG. 7F) Tumor infiltrating lymphocytes were re-stimulated with dendritic cells loaded with tumor lysates and assessed for expression of IFN gamma by intracellular cytokine staining.

FIGS. 8A-8D. NDV infection is restricted to the injected tumor. FIG. 8A) Recombinant NDV-Fluc was administered intratumorally (IT) or intravenously (IV) into Balb/C animals bearing CT26 tumors and images were acquired over the next 72 hours. FIG. 8B) NDV-Fluc was administered to C57BL/6 mice bearing bilateral B16F10 melanoma tumors and animals were monitored for 120 hours. Representative luminescence images are shown. FIG. 8C) Quantification of luminescence from the tumor site normalized to background luminescence. FIG. 8D) Area under the curve (AUC) calculated from the data in panel (FIG. 8C). Data show representative results from 1 of 3 independent experiments with 3-5 mice/group. ***p<0.001 (p<0.05 indicates statistical significance).

FIGS. 9A-9F. NDV infection increases tumor leukocyte infiltration in the virus-injected tumors. Animals were treated according to the scheme described in FIG. 10A. Tumors were excised on day 15, and TILs were labeled and analyzed by flow cytometry. FIG. 9A) Representative flow cytometry plots of percentages of tumor-infiltrating CD45+ and CD3+ cells. FIG. 9B) Absolute numbers of CD45+ cells/g tumor. FIG. 9C) Absolute numbers of innate immune cells/g tumor. FIG. 9D) Representative plots of percentages of CD4+FoxP3+ (Treg) and CD4+FoxP3− (T conv) cells. FIG. 9E) Absolute numbers of conventional and regulatory CD4+ cells and CD8+ cells/g tumor. FIG. 9F) Calculated Tconv/Treg and CD8+/Treg ratios from the tumors. Data represent cumulative results from 3 independent experiments with 3-5 mice/group. Mean+/−SEM is shown. *p<0.05, **p<0.01, ***p<0.001, ****p<0.0001.

FIGS. 10A-10M. NDV increases distant tumor lymphocyte infiltration and delays tumor growth. FIG. 10A) Treatment scheme. FIG. 10B) Representative flow cytometry plots of percentages of tumor-infiltrating CD45+ and CD3+ cells. FIG. 10C) Absolute numbers of CD45+ cells/g tumor. FIG. 10D) Absolute numbers of innate immune cells/g tumor. FIG. 10E) Tumor sections from distant tumors were stained with H&E (upper panels) or labeled for CD3 and FoxP3 (bottom panels) and analyzed by microscopy. Areas denoted by arrows indicate areas of necrosis and inflammatory infiltrates. Scale bars represent 200 μm. FIG. 10F) Representative flow cytometry plots of percentages of CD4+FoxP3+ (Treg) and CD4+FoxP3− (Tconv) cells. FIG. 10G) Absolute numbers of conventional and regulatory CD4+ cells and CD8+ cells/g tumor calculated from flow cytometry. FIG. 10H) Relative percentages of Tregs out of CD45+ cells.

FIG. 10I) Calculated Tconv/Treg and CD8+/Treg ratios. (FIG. 10J and FIG. 10K) Upregulation of ICOS, Granzyme B, and Ki-67 on tumor-infiltrating Tconv (FIG. 10J) and CD8+ cells (FIG. 10K). FIG. 10L) Growth of NDV-injected and distant tumors. FIG. 10M) Overall animal survival. Data represent cumulative results from 3 (FIG. 10B-10K) or 2 (FIGS. 10L-10M) independent experiments with n=3-5 per group. Mean+/−SEM is shown. *p<0.05, **p<0.01, ***p<0.001, ****p<0.0001.

FIGS. 11A-11E. NDV therapy increases distant tumor lymphocyte infiltration in bilateral footpad melanoma model. Animals bearing bilateral footpad melanoma tumors were treated according to the schedule described in FIG. 10A. Distant tumors were excised on day 15 and TILs were labeled and analyzed by flow cytometry. FIG. 11A) Representative flow cytometry plots of percentages of tumor-infiltrating CD45+ and CD3+ cells. FIG. 11B) Representative flow cytometry plots of percentages of CD4+FoxP3+ and CD4+FoxP3− cells. FIG. 11C) Absolute numbers of conventional and regulatory CD4+ cells and CD8+ cells/g tumor. FIG. 11D and FIG. 11E) Upregulation of ICOS, Granzyme B, and Ki-67 on tumor-infiltrating CD8+ (FIG. 11D) and Tconv (FIG. 11E) lymphocytes. Data show representative results from 1 of 2 independent experiments with 5 mice/group. *p<0.05, **p<0.01, ***p<0.001, ****p<0.0001.

FIGS. 12A-121. NDV induces infiltration of adoptively-transferred tumor-specific lymphocytes and facilitates tumor inflammation. FIG. 12A) Treatment scheme. FIG. 12B) Representative luminescence images from animals treated with NDV and adoptively-transferred Trp1-Fluc lymphocytes. FIG. 12C) Quantification of average luminescence from the tumor sites. FIG. 12D) The area under the curve (AUC) calculated from the data in panel FIG. 12C. FIG. 12E) Absolute number of Pmel lymphocytes from distant tumors calculated from flow cytometry. FIG. 12F) Representative flow cytometry plots of percentages of CD45+ and CD3+ cells infiltrating distant tumors of animals treated per treatment scheme in FIG. 12A. FIG. 12G) Experimental scheme for serum transfer from animals treated intratumorally with single injection of NDV or PBS. FIG. 12H) Representative flow cytometry plots of percentages of CD45+ and CD3+ cells infiltrating serum-injected tumors. FIG. 12I) Absolute numbers of the indicated cell subsets in serum-injected tumors calculated from flow cytometry. Data for FIGS. 12B-12E represent 1 of 3 experiments with n=4-5 per group. Data for FIGS. 12G-121 represent pooled data from 2 independent experiments with n=5 per group. Mean+/−SEM is shown. *p<0.05, **p<0.01, ***p<0.001, ****p<0.0001.

FIG. 13. Intratumoral NDV provides protection from tumor rechallenge. Animals cured of B16F10 melanoma by NDV were injected on day 75 with 1×10⁵ B16F10 melanoma cells, monitored for tumor growth, and euthanized when the tumors reached 1000 mm3. Overall animal survival is shown. Data show cumulative results from 1 of 2 independent experiments with 10 mice/group. ****p<0.0001.

FIGS. 14A-14B. Tumor-infiltrating CD8+ lymphocytes upregulate CTLA-4 in response to NDV therapy. Representative dot plots (left) and cumulative results (right) of CTLA-4 expression in CD8+ cells in NDV-treated (FIG. 14A), and distant (FIG. 14B) tumors. Representative results from 1 of 3 experiments with 3 mice per group. *p<0.05.

FIGS. 15A-15B. NDV therapy leads to upregulation of PD-L1 on tumors and tumor-infiltrating leukocytes. FIG. 15A) PD-L1 expression on B16F10 cells infected in vitro (left panel), and in vivo in virus injected and distant tumors. Top, representative flow cytometry histograms, bottom, average median fluorescence intensity (MFI) of PD-L1 expression on B16F10 cells from tumors. FIG. 15B) PD-L1 expression on the surface of tumor-infiltrating leukocytes isolated from distant tumors. Top: representative flow cytometry histograms, bottom: calculated average MFI for each cell subset.

FIGS. 16A-16D. Combination therapy of NDV with antibodies blocking PD-1 leads to enhanced anti-tumor efficacy in bilateral flank B16 melanoma model. FIG. 16A) Treatment scheme. FIG. 16B) Right flank (NDV-injected) tumor growth. FIG. 16C) Left flank (distant) tumor growth. FIG. 16D) Overall survival.

FIGS. 17A-17D. Combination therapy of NDV with antibodies blocking PD-L1 leads to enhanced anti-tumor efficacy in bilateral flank B16 melanoma model. FIG. 17A) Treatment scheme. FIG. 17B) Right flank (NDV-injected) tumor growth. FIG. 17C) Left flank (distant) tumor growth. FIG. 17D) Overall survival.

FIGS. 18A-18E. Combination therapy with NDV and anti-PD-1 therapy results in increased distant tumor infiltration with effector but not regulatory T cells. FIG. 18A) Representative flow cytometry plots of percentages of CD4+ and CD8+ cells in tumors. FIG. 18B) Representative flow cytometry plots of percentages of Tconv (CD4+FoxP3−) and Treg (CD4+FoxP3+) cells. FIG. 18C) Absolute numbers of T cell subsets per gram of tumor, calculated from flow cytometry. FIG. 18D) Relative percentages of Tregs from CD4+ T cells. FIG. 18E) Calculated Tconv/Treg and CD8/Treg ratios.

FIGS. 19A-19B. TILs from distant tumors in animals treated with combination NDV and anti-PD-1 therapy upregulate lytic and proliferation markers. FIG. 19A) Representative flow cytometry plots of percentages of Tconv and CD8 lymphocytes positive for Granzyme B and Ki67. FIG. 19B) Percentages of Tconv and CD8+ T cells positive for Granzyme B and Ki67.

FIGS. 20A-20C. NDV induces tumor immune infiltration and upregulation of ICOS on CD4 and CD8 cells in the virus-injected and distant tumors. FIG. 20A) Treatment scheme. FIG. 20B) Expression of ICOS on tumor-infiltrating CD4+FoxP3− and CD8+ cells isolated from NDV-injected (right flank) tumors. Representative flow cytometry plots (top) and median fluorescence intensities (MFI) (bottom) are shown. FIG. 20C) Expression of ICOS on tumor-infiltrating CD4+FoxP3− and CD8+ cells isolated from distant (left flank) tumors. Representative flow cytometry plots (top) and median fluorescence intensities (MFI) (bottom) are shown.

FIG. 21: Schematic of pT7NDV-LS-L289A plasmid.

FIGS. 22A-22D: Enhanced anti-tumor efficacy with intratumoral NDV-muIL-12 in combination with anti-muPD-1 mAb muDX400 in B16F10 bilateral tumor model. Mouse B16F10 cells were subcutaneously implanted into the right flanks (2×10⁵ cells) and left flanks (1×10⁵ cells) of immunocompetent C57BL/6J mice. Animals were assigned into groups 9 days after implantation (Day 0) based on tumor volume (TV) in the right flanks (injected tumors) with median TV=65 mm³. The median TV in the left flanks (non-injected tumors) of the 6 groups ranged from 31 to 38 mm³. Dosing was initiated on Day 0. Mouse IgG1 isotype control and muDX400 at 10 mg/kg were administered intraperitoneally every 4 days for a total of 3 doses. PBS, NDV WT, and NDV-muIL-12 at 1× 10⁷ pfu were administered into the tumors on the right flanks every 2 days for a total of 4 doses. There were 10 animals in each group. FIG. 22A-22B: growth curves for injected tumors and non-injected tumors, respectively, are presented. Tumor volumes (“TVs”) are presented as median with 68% confidence intervals. FIGS. 22C and 22D: individual animal TV on Day 13 for injected and non-injected tumors, respectively, arc presented. Dotted lines indicate Day 0 median TVs. P-values for key comparisons are indicated (p<0.05 indicates statistical significance). Group 1: mIgG1 control+PBS; Group 2: muDX400+PBS; Group 3: mIgG1 control+NDV-WT; Group 4: muDX400+NDV-WT; Group 5: mIgG1 control+NDV-muIL-12; Group 6: muDX400+NDV-muIL-12. For injected tumors, the number of complete regressions were as follows: Group 1:0; Group 2:0; Group 3:1; Group 4:1; Group 5:2; and Group 6:6. For non-injected tumors, the number of complete regressions were as follows: Group 1:0; Group 2:1; Group 3:1; Group 4:3; Group 5:0; and Group 6:5.

FIGS. 23A-23D: Induction of immune genes in injected tumors with intratumoral NDV-muIL 12 in combination with anti-muPD-1 mAb muDX400 in B16F10 bilateral tumor model as described in legend for FIGS. 22A-22D. Experimental design is described in Sections 6.3.1.9 and 6.3.1.13. Tumors were harvested on Day 14, and gene expression in tumor tissues were assessed by RTqPCR and normalized to ubiquitin. Individual animal expression levels of genes for T cell markers (FIG. 23A), cytokines (FIG. 23B), IFN-inducible genes (FIG. 23C), and Pd-1 and Pd-11 (FIG. 23D) in injected tumors are presented. Lines denote mean values. For comparisons between Groups 3 and 5, 4 and 6, and 5 and 6, p-values<0.05 are indicated.

FIGS. 24A-24D: Induction of immune genes in non-injected tumors with intratumoral NDV-muIL-12 in combination with anti-muPD-1 mAb muDX400 in B16F10 bilateral tumor model as described in legend for FIGS. 22A-22D. Experimental design is described in Sections 6.3.1.9 and 6.3.1.13. Tumors were harvested on Day 14, and gene expression in tumor tissues were assessed by RTqPCR and normalized to ubiquitin. Individual animal expression levels of genes for T-cell markers (FIG. 24A), cytokines (FIG. 24B), and IFN-inducible genes (FIG. 24C), and Pd-1 and Pd-11 (FIG. 24D) in non-injected tumors are presented. Lines denote mean values. P-values<0.05 are indicated.

FIGS. 25A and 25B: Treatment with NDV-mull, 12 in combination with anti-muPD-1 mAb muDX400 increased survival of B16F10-bearing animals. Mouse B16-F10 cells were subcutaneously implanted into the right flanks (2×10⁵ cells) and left flanks (1×10⁵ cells) of immunocompetent C57BL/6J mice. Animals were assigned into groups 7 days after implantation (Day 0) based on tumor volume (TV) in the right flanks (injected tumors) with median TV=56 mm³. The median TV in the left flanks (non-injected tumors) was 38 mm³. Dosing was initiated on Day 0. Mouse IgG1 isotype control and muDX400 at 10 mg/kg were administered intraperitoneally every 4 days for a total of 3 doses. PBS, NDV-WT, and NDV-muIL-12 at 1× 10⁷ pfu were administered into the tumors on the right flanks every 2 days for a total of 4 doses. Animals were followed up to Day 54. Animals were euthanized when the sum of volumes of injected and non-injected tumors was ≥2000 mm³ or body weight loss was ≥ 20%. There were 10 animals in each group. FIG. 25A shows survival curves for Groups 1 (mIgG1 control+PBS), 3 (mIgG1 control+NDV-WT), and 5 (mIgG1 control+NDV-muIL-12). FIG. 25B shows survival curves for Groups 2 (muDX400+PBS), 4 (muDX400+NDV-WT), and 6 (muDX400+NDV-muIL-12).

FIGS. 26A and 26B: Increased infiltration of CD3+ T cells in injected and non-injected tumors with intratumoral NDV-muIL-12 in combination with anti-muPD-1 mAb muDX400 in B16F10 bilateral tumor model. Mouse B16F10 cells were subcutaneously implanted into the right flanks (2×10⁵ cells) and left flanks (1× 10⁵ cells) of immunocompetent C57BL/6J mice. Animals were assigned into groups 8 days after implantation (Day 0) based on tumor volume (TV) in the right flanks (injected tumors) with mean TV=108 mm³. The mean TV=63 mm³ in the left flanks. Dosing was initiated on Day 0. Mouse IgG1 isotype control and muDX400 at 10 mg/kg were administered intraperitoneally every 6 days for a total of 2 doses. PBS, NDV WT, and NDV-muIL-12 at 1×10⁷ pfu were administered into the tumors on the right flanks every 2 days for a total of 4 doses. Experimental design is described in Sections 6.3.1.9 and 6.3.1.10. Representative images are shown for injected tumors (FIG. 26A) and non-injected tumors (FIG. 26B) on Day 8.

FIG. 27A: Lytic activity of NDV-hulL-12 in panel of 26 human cancer cell lines: melanoma (n=3), head and neck squamous cell carcinoma (HNSCC, n=4), lung (n=4), breast (n=3), ovarian (n=3), colon (n=4), and pancreatic (n=5). Cells were infected with NDV-huIL-12 at MOI of 2 and 6, and cell viability was determined 48 hours following infection. Viability is expressed as the percentage of viable cells in infected cells relative to uninfected cells. Treatment with 10 μM puromycin in DMSO was included as a positive control for cell killing; viability is expressed as the percentage of viable cells relative to treatment with DMSO. Values are presented as mean of 8 replicates #standard error of the mean. The dotted line indicates cut-off of 20% reduction in cell viability. DMSO=dimethyl sulfoxide. MOI=multiplicity of infection.

FIG. 27B: Induction of IL-12p70, IFN-γ, and IP-10 in panel of 26 human cancer cell lines. Supernatants were harvested 48 hours following mock infection (white bars) or infection with NDV-huIL-12 (MOI=2) (black bars) in a range of human tumor cell lines: melanoma (n=3), head and neck squamous cell carcinoma (HNSCC, n=4), lung (n=4), breast (n=3), ovarian (n=3), colon (n=4), and pancreatic (n=5). Supernatants were tested for protein concentrations of various cytokines and chemokines using immunoassays. Shown are the mean of 4 replicates 1 standard error of the mean for IL-12p70 (top), IFN-β (middle), and IP-10 (bottom). Dotted lines indicate LLOQ and ULOQ: IL, 12p70 (LLOQ=5 pg/mL and ULOQ=5580 pg/mL), IFN-β (LLOQ=98 pg/mL), and IP-10 (LLOQ-2 pg/mL and ULOQ=9040 pg/mL). LLOQ=lower limit of quantitation; MOI-multiplicity of infection; and ULOQ-upper limit of quantitation.

FIG. 28: Induction of IFN-α-2a, IL-12, IFN-γ, and IP-10 in human tumor histoculture with treatment with NDV-huIL-12. Samples of renal cell carcinoma (RCC, n=4), colorectal carcinoma (CRC, n=3), breast carcinoma (n=2), and head and neck squamous cell carcinoma (HNSCC; n=1) were untreated (media) or treated with 3×10⁷ pfu NDV-WT or NDV-huIL-12 for up to 48 hours. Supernatants were collected from the histoculture for assessment of protein concentrations of various cytokines and chemokines using immunoassays. Shown are the mean±standard error of the mean (Wilcoxon signed rank test, p-values<0.05 are indicated) for IFN-α-2a, IFN-β, IL-12p70, IFN-γ, and IP-10. Dotted lines indicate LLOQ values: IFN-α-2a=2.4 pg/mL, IFN-β=24 pg/mL, IL-12p70=2.4 pg/mL. With the exception of IP-10, where supernatants were collected at 24 hours and assayed at 1:15 dilution, data are shown for undiluted supernatant collected at 48-hour time point. LLOQ=lower limit of quantitation.

FIGS. 29A-29D: Induction of gene expression of IFN-inducible genes, chemokines, Il-12p40, Ifn-γ, and Pd-11 (gene encoding PD-L1) in human tumor histoculture with treatment with NDV-huIL-12. Samples of renal cell carcinoma (RCC, n=4), colorectal carcinoma (CRC, n=3), breast carcinoma (n=2), and head and neck squamous cell carcinoma (HNSCC, n=1) were untreated (media) or treated with 3×10⁷ pfu NDV-WT or NDV-huIL-12 for up to 48 hours. The samples were snap-frozen, and following RNA isolation, gene expression of panel of immune genes was analyzed using the Fluidigm RTqPCR platform. Shown are the mean of the values normalized to ubiquitin+standard error of the mean (Wilcoxon signed rank test, p values<0.05 are indicated) for IFN-inducible genes (FIG. 29A), chemokines (FIG. 29B), Il-12p40 and Ifn-γ (FIG. 29C), and Pd-11 (FIG. 29D) for 48-hour time point.

FIG. 30: Induction of IFN-α-2a, IL-12p70, IFN-γ, and IP-10 in human whole blood with NDV-huIL 12 treatment. Whole blood (1 mL) from patients with solid cancers (n=5) and normal healthy donors (n=5) were untreated (media) or treated with 3×10⁷ pfu NDV-WT or NDV-huIL-12 for 48 hours. Plasma was collected for assessment of protein concentrations of various cytokines and chemokines using immunoassays. Shown are the mean I standard error of the mean (Wilcoxon signed rank test, p values<0.05 are indicated) for IFN-α-2a, IFN-β, IL-12p70, IFN-γ, and IP-10. Dotted lines indicate either LLOQ or ULOQ values: IFN-α-2a (LLOQ)=2.4 pg/mL, IFN-β (LLOQ)=24 μg/mL, IP-10 (ULOQ)=2610 μg/mL. Values are shown for undiluted supernatant collected at the 48-hour time point. LLOQ=lower limit of quantitation; ULOQ=upper limit of quantitation.

FIGS. 31A-31D: Induction of gene expression of IFN-inducible genes, chemokines, Il-12p40, Ifn-γ, and Pd-11 in human whole blood with NDV-huIL 12 treatment. Whole blood (4 mL) from patients with solid cancers (n=5) and normal healthy donors (n=5) were untreated (media) or treated with 12×10⁷ pfu NDV-WT or NDV-huIL-12 for 24 hours. Whole blood was collected into PAXgene blood RNA tubes, and following RNA isolation, gene expression of panel of immune genes was analyzed using the Fluidigm RTqPCR platform. Shown are the mean of the values normalized to ubiquitin 1 standard error of the mean (Wilcoxon signed rank test, p-values<0.05 are indicated) for IFN-inducible genes (FIG. 31A), chemokines (FIG. 31B), Il-12p40 and Ifn-γ (FIG. 31C), and Pd-11 (FIG. 31D) for 24-hour time point.

FIG. 32: Analysis of NDV-huIL-12 by flow virometry.

FIG. 33: Analysis of NDV-huIL-12 by reducing SDS-PAGE. MW: Molecular weight marker; CB: clarified bulk; and SFP: sterile filtered product.

FIG. 34: MOI-dependent huIL-12 expression curve following 24-hour Vero cell infection by NDV-huIL-12.

FIG. 35: huIL-12 induced receptor dimerization using PathHunter® Bioassay Detection kit. Each curve represents the titration of hull-12 produced by Vero cells following 24-hour infection of NDV-huIL-12 at the indicated MOI.

FIG. 36: rNDV-muIL12 Expression in BSRT7 cells and allantoic fluid. Y axis is concentration of muIL-12 in pg/mL as determined using the commercially available ELISA kit (Mouse IL-12p70 Quantikine ELISA Kit, R&D Systems, Catalog No. M1270). From left to right: BSRT7 cells infected with NDV-WT (negative control), NDV-muIL-12 (MOI=3, 24 hpi), NDV-muIL-12 (MOI=10, 24 hpi), NDV-muIL-12 (MOI=3, 48 hpi), or NDV-muIL-12 (MOI=10, 48 hpi), or allantoic fluid from NDV-muIL-12 infected eggs. MOI=multiplicity of infection. Hpi=hours post-infection.

FIGS. 37A-37N: Enhanced anti-tumor efficacy with NDV-muIL-12 versus NDV-muIL-23 or NDV-muIL-27 in combination with anti-muPD-1 mAb muDX400 in B16F10 bilateral tumor model. Mouse B16F10 cells were subcutaneously implanted into the right flanks (2×10⁵ cells) and left flanks (1×10⁵ cells) of immunocompetent C57BL/6J mice. Animals were assigned into groups 9 days after implantation (Day 0) based on tumor volume (TV) in the right flanks (injected tumors) with median TV=65 mm³. The median TV in the left flanks (non-injected tumors) of the 6 groups ranged from 31 to 43 mm³. Dosing was initiated on Day 0. Mouse IgG1 isotype control and muDX400 at 10 mg/kg were administered intraperitoneally every 4 days for a total of 3 doses. PBS, NDV-WT, NDV-muIL-12, NDV-muIL-23, and NDV-muIL-27 at 1×10⁷ pfu were administered into the tumors on the right flanks every 2 days for a total of 5 doses. There were 10 animals in each group. Individual animal growth curves for injected are presented: Group 1: mIgG1 control+PBS (FIG. 37A); Group 2: muDX400+PBS (FIG. 37B); Group 3: muDX400+NDV-WT (FIG. 37C); Group 4: muDX400+NDV-muIL-12 (FIG. 37D); Group 5: muDX400+NDV-muIL-23 (FIG. 37E); Group 6: muDX400+NDV-muIL-27 (FIG. 37F). Day 14 tumor volume (median with 68% confidence intervals) and the number of complete and partial regressions for injected tumors are presented in FIG. 37G. Individual animal growth curves for non-injected are presented: Group 1: mIgG1 control+PBS (FIG. 37H); Group 2: muDX400+PBS (FIG. 37I); Group 3: muDX400+NDV-WT (FIG. 37J); Group 4: muDX400+NDV-muIL-12 (FIG. 37K); Group 5: muDX400+NDV-muIL-23 (FIG. 37L); Group 6: muDX400+NDV-muIL-27 (FIG. 37M). Day 14 tumor volume (median with 68% confidence intervals) and the number of complete and partial regressions for non-injected tumor are presented in FIG. 37N.

FIGS. 38A-38J: Enhanced anti-tumor efficacy with NDV-muIL-12 versus NDV-muIL-2 with anti-muPD-1 mAb muDX400 in B16F10 bilateral tumor model. Mouse B16F10 cells were subcutaneously implanted into the right flanks (2×10⁵ cells) and left flanks (1×10⁵ cells) of immunocompetent C57BL/6J mice. Animals were assigned into groups 10 days after implantation (Day 0) based on tumor volume (TV) in the right flanks (injected tumors) with median TV=55 mm³. The median TV in the left flanks (non-injected tumors) was 34 mm³. Dosing was initiated on Day 0. Mouse IgG1 isotype control and muDX400 at 10 mg/kg were administered intraperitoneally every 4 days for a total of 5 doses. PBS, NDV-WT, NDV-muIL-12, and NDV-muIL-2 at 1×10⁷ pfu were administered into the tumors on the right flanks every 2 days for a total of 4 doses. There were 12 animals in each group. Individual animal growth curves for injected are presented: Group 1: mIgG1 control+PBS (FIG. 38A); Group 2: muDX400+NDV-WT (FIG. 38B); Group 3: muDX400+NDV-muIL-12 (FIG. 38C); Group 4: muDX400+NDV-muIL-2 (FIG. 38D). Individual animal growth curves for non-injected are presented: Group 1: mIgG1 control+PBS (FIG. 38F); Group 2: muDX400+NDV-WT (FIG. 38F); Group 3: muDX400+NDV-muIL-12 (FIG. 38G); Group 4: muDX400+NDV-muIL-2 (FIG. 38H). For injected and non-injected tumors, tumor volume of the last measurement (median with 68% confidence intervals) and the number of complete and partial regressions are presented in FIG. 38I and FIG. 38J, respectively.

FIG. 39: Induction of NDV-huIL-12 response signature in human tumor histoculture with treatment with NDV-huIL-12. Samples of renal cell carcinoma (RCC, n=7) and colorectal carcinoma (CRC, n=7) were untreated (media) or treated with 3×10⁷ pfu NDV-huIL-12 for up to 48 hours. The samples were snap-frozen, and following RNA isolation, gene expression of panel of immune genes was analyzed using the Fluidigm Biomark RTqPCR platform. Shown are the mean fold change in expression between NDV-huIL-12 and untreated (media) at 6, 24 and 48 hours; values normalized to ubiquitin were used in calculating fold change.

FIGS. 40A-B: Induction of the T-cell inflamed, IFN-γ-related gene signature (18-GEP) score with NDV and NDV-huIL-12 in both GEP-negative and GEP-positive tumors. Samples of renal cell carcinoma (RCC, n=10), colorectal carcinoma (CRC, n=4), breast carcinoma (n=2), non-small cell lung carcinoma (n=2), and head and neck squamous cell carcinoma (HNSCC, n-1) were untreated (media) or treated with either 3×10⁷ pfu NDV-WT or 3×10⁷ pfu NDV-huIL-12 for up to 24 hours. The samples were snap-frozen, and following RNA isolation, 18-gene GEP signature was analyzed using the NanoString platform. Shown are the mean GEP score with standard deviation and p-value (ANOVA Friedman Test, followed by Dunn's multiple comparison test) (FIG. 40A) and individual GEP score (FIG. 40B).

FIGS. 41A-D: Induction of Type I interferons and IP-10 by recombinant IL-12 and NDV-huIL 12. Histoculture samples from renal cell carcinoma (RCC, n=6), colorectal carcinoma (CRC, n=1), and non-small cell lung cancer (NSCLC, n=2) were untreated (media) or treated with 3×10⁷ pfu NDV-WT, 3×10⁷ NDV-huIL-12, or recombinant human IL-12 (10, 25, and 50 ng/mL) for up to 48 hours. The supernatants were collected and analyzed for secretion of IL-12p70 (FIG. 41A), IFN-α-2a (FIG. 41B), IFN-β (FIG. 41C), and IP-10 (FIG. 41D). Shown are the mean value with SEM and p-value (ANOVA Friedman Test, followed by Dunn's multiple comparison test).

### 5. DETAILED DESCRIPTION

In one aspect, presented herein are methods for treating cancer utilizing an NDV described herein (e.g., an NDV or a chimeric NDV described in Section 5.1 or Section 5.2, infra) or a composition comprising such a chimeric NDV. In a specific embodiment, a method for treating cancer comprises infecting a cancer cell in a subject with a chimeric NDV described herein (e.g., a chimeric NDV described in Section 5.2, infra) or a composition thereof. In another embodiment, a method for treating cancer comprises administering to a subject in need thereof a chimeric NDV described herein (e.g., a chimeric NDV described in Section 5.2, infra) or a composition thereof. In specific embodiments, an effective amount of a chimeric NDV described herein (e.g., a chimeric NDV described in Section 5.2, infra) or a composition comprising an effective amount of a chimeric NDV described herein is administered to a subject to treat cancer. In a specific embodiment, the chimeric NDV comprises a packaged genome that encodes IL-12 (e.g., the IL-12 p35 and IL-12 p40 subunits). In a specific embodiment, the IL-12 (e.g., human IL-12) is expressed by cells infected with the chimeric NDV. In certain embodiments, the genome of the NDV also encodes a mutated F protein. In certain embodiments, two or more chimeric NDVs are administered to a subject to treat cancer.

In another embodiment, a method for treating cancer comprises administering to a subject in need thereof cancer cells infected with an NDV described herein (e.g., an NDV or a chimeric NDV described in Section 5.1 and/or Section 5.2, infra) or composition thereof. In specific embodiments, the cancer cells have been treated with gamma radiation prior to administration to the subject or incorporation into the composition. In another embodiment, a method for treating cancer comprises administering to a subject in need thereof a protein concentrate or plasma membrane fragments from cancer cells infected with a chimeric NDV (e.g., a chimeric NDV described in Section 5.2, infra) or a composition thereof. In specific embodiments, the chimeric NDV comprises a packaged genome which encodes IL-12 (e.g., human IL-12), wherein the IL-12 is expressed by the NDV. In certain embodiments, the genome of the NDV also encodes a mutated F protein, which is expressed by the NDV

In another aspect, presented herein are methods for treating cancer utilizing an NDV described herein (e.g., a chimeric NDV such as described in Section 5.2, infra) or a composition comprising the NDV in combination with one or more other therapies. In one embodiment, presented herein are methods for treating cancer comprising administering to a subject (e.g., a human subject) an NDV described herein (e.g., a chimeric NDV, such as described in Section 5.2, infra) and one or more other therapies. In another embodiment, presented herein are methods for treating cancer comprising administering to a subject (e.g., a human subject) an effective amount of an NDV described herein or a composition comprising an effective amount of an NDV described herein, and one or more other therapies. In a specific embodiment, presented herein are uses of an NDV described herein (e.g., a chimeric NDV such as described in Section 5.2, infra) in the preparation of a medicament for use in combination with one or more other therapies for treating cancer in a subject (e.g., a human subject). In another specific embodiment, presented herein are an NDV described herein (e.g., a chimeric NDV such as described in Section 5.2, infra) for use in a method for treating cancer in a subject (e.g., a human subject), wherein the method further comprises administering one or more other therapies.

In a preferred embodiment, the one or more therapies includes an antagonist of PD-1 or a ligand thereof (e.g., an anti-PD-1 antibody that blocks the interaction between PD-1 and a ligand thereof (e.g., PD-L1, PD-L2 or both PD-L1 and PD-L2) or anti-PD-1 L1 antibody that blocks the interaction between PD-L1 and PD-1). The NDV and one or more other therapies can be administered concurrently or sequentially to the subject. In certain embodiments, the NDV and one or more other therapies are administered in the same composition. In other embodiments, the NDV and one or more other therapies are administered in different compositions. The NDV and one or more other therapies can be administered by the same or different routes of administration to the subject.

In another aspect, presented herein are methods for treating cancer utilizing a chimeric NDV described herein (e.g., a chimeric NDV such as described in Section 5.2, infra), or a composition comprising such the chimeric NDV, in combination with an antagonist of PD-1 or a ligand thereof, wherein the chimeric NDV comprises a packaged genome comprising a transgene encoding IL-12 (e.g., human IL-12). In one embodiment, presented herein are methods for treating cancer comprising administering to a subject (e.g., a human subject) a chimeric NDV described herein (e.g., a chimeric NDV, such as described in Section 5.2, infra) and an antagonist of PD-1 or a ligand thereof, wherein the chimeric NDV comprises a packaged genome comprising a transgene encoding IL-12 (e.g., human IL-12). In another embodiment, presented herein are methods for treating cancer comprising administering to a subject (e.g., a human subject) an effective amount of a chimeric NDV described herein or a composition comprising an effective amount of a chimeric NDV described herein, and an antagonist of PD-1 or a ligand thereof, wherein the chimeric NDV comprises a packaged genome comprising a transgene encoding IL-12 (e.g., human IL-12). The chimeric NDV and antagonist of PD-1 or a ligand thereof can be administered concurrently or sequentially to the subject. In certain embodiments, the chimeric NDV and antagonist of PD-1 or a ligand thereof are administered in the same composition. In other embodiments, the chimeric NDV and antagonist of PD-1 or a ligand thereof are administered in different compositions. The chimeric NDV and antagonist of PD-1 or a ligand thereof can be administered by the same or different routes of administration to the subject. In a specific embodiment, the chimeric NDV is administered intratumorally and the antagonist is administered intravenously.

In one embodiment, presented herein are uses of a chimeric NDV in the preparation of a medicament for use in combination with an antagonist of PD-1 or a ligand thereof for use in treating cancer in a subject (e.g., a human subject) a chimeric NDV described herein, wherein the chimeric NDV comprises a packaged genome comprising a transgene encoding IL-12 (e.g., human IL-12). In another embodiment, presented herein are a chimeric NDV for use in a method for treating cancer in a subject (e.g., a human subject), wherein the chimeric NDV comprises a packaged genome comprising a transgene encoding IL-12 (e.g., human IL-12), and wherein the method further comprises administering an antagonist of PD-1 or a ligand thereof. The chimeric NDV and antagonist of PD-1 or a ligand thereof can be administered concurrently or sequentially to the subject. In certain embodiments, the chimeric NDV and antagonist of PD-1 or a ligand thereof are administered in the same composition. In other embodiments, the chimeric NDV and antagonist of PD-1 or a ligand thereof are administered in different compositions. The chimeric NDV and antagonist of PD-1 or a ligand thereof can be administered by the same or different routes of administration to the subject. In a specific embodiment, the chimeric NDV is administered intratumorally and the antagonist is administered intravenously.

International Patent Application Publication No. WO 2014/158811 and U.S. Patent Application Publication Nos. 2016/0015760 A1 and 2014/0271677 A1 are each incorporated by reference herein in their entireties. For example, the summary, the description of NDVs, the description of chimeric NDVs, the description of compositions, the description of routes of administration, and the description of anti-cancer and other uses described in, e.g., Sections 3, 5.1, 5.2, 5.5, and 5.6, of International Patent Application Publication No. WO 2014/158811 and U.S. Patent Application Publication Nos. 2016/0015760 A1 and 2014/0271677 A1 are incorporated by reference herein in their entireties.

### 5.1 Newcastle Disease Virus

Any NDV type or strain may be used in a combination therapy disclosed herein, including, but not limited to, naturally-occurring strains, variants or mutants, mutagenized viruses, reassortants and/or genetically engineered viruses. One skilled in the art would understand that viruses may undergo mutation when cultured, passaged or propagated. The NDV may contain these naturally occurring mutations, in addition to mutations introduced for cloning purposes. The NDV may be a homogenous or heterogeneous population with none, or one or more of these mutations. In a specific embodiment, the NDV used in a combination therapy disclosed herein is a naturally-occurring strain. In certain embodiments, the NDV is a lytic strain. In other embodiments, the NDV used in a combination therapy disclosed herein is a non-lytic strain. In certain embodiments, the NDV used in a combination therapy disclosed herein is lentogenic strain. In some embodiments, the NDV is a mesogenic strain. In other embodiments, the NDV used in a combination therapy disclosed herein is a velogenic strain. See, e.g., Newcastle Disease, Avian Paramyoxvirus-1 Infection, Goose Paramyoxvirus Infection, Ranikhet disease, the Center for Food Security & Public Health, Iowa State University, Institute for International Cooperation in Animal Biologics, College of Veterinary Medicine, Iowa State University, pp. 1-9 (January 2016) for a discussion regarding lentogenic, mesogenic and velogenic NDV strains, which is incorporated herein by reference in its entirety. Specific examples of NDV strains include, but are not limited to, the 73-T strain, NDV HUJ strain, Ulster strain (see, e.g., GenBank No. U25837), MTH-68 strain, Italien strain (see, e.g., GenBank No. EU293914), Hickman strain (see, e.g., Genbank No. AF309418), PV701 strain, Hitchner B1 strain (see, e.g., GenBank No. AF309418 or NC_002617), La Sota strain (see, e.g., GenBank Nos. AY845400 and JF950510.1 and GI No. 56799463), YG97 strain (see, e.g., GenBank Nos. AY351959 or AY390310), MET95 strain (see, e.g., GenBank No. AY143159), Roakin strain (see, e.g., GenBank No. AF124443), and F48E9 strain (see, e.g., GenBank Nos. AF163440 and U25837). In a specific embodiment, the NDV used in a combination therapy disclosed herein that is the Hitchner B1 strain. In another specific embodiment, the NDV used in a combination therapy disclosed herein is a B1 strain as identified by GenBank No. AF309418 or NC_002617. In another specific embodiment, the NDV used in a combination therapy disclosed herein is the NDV identified by ATCC No. VR2239. In another specific embodiment, the NDV used in a combination therapy disclosed herein is the La Sota strain. In a specific embodiment, the nucleotide sequence of the La Sota genome is as set forth in SEQ ID NO: 50. One skilled in the art will understand that the NDV genomic RNA sequence is the reverse complement of a cDNA sequence encoding the NDV genome. Thus, any program that generates a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar).

In specific embodiments, the NDV used in a combination therapy disclosed herein is not pathogenic in birds as assessed by a technique known to one of skill. In certain specific embodiments, the NDV used in a combination therapy is not pathogenic as assessed by intracranial injection of 1-day-old chicks with the virus, and disease development and death as scored for 8 days. In some embodiments, the NDV used in a combination therapy disclosed herein has an intracranial pathogenicity index of less than 0.7, less than 0.6, less than 0.5, less than 0.4, less than 0.3, less than 0.2 or less than 0.1. In certain embodiments, the NDV used in a combination therapy disclosed herein has an intracranial pathogenicity index of zero.

In certain embodiments, the NDV used in a combination therapy disclosed herein is a mesogenic strain that has been genetically engineered so as not be a considered pathogenic in birds as assessed by techniques known to one skilled in the art. In certain embodiments, the NDV used in a combination therapy disclosed herein is a velogenic strain that has been genetically engineered so as not be a considered pathogenic in birds as assessed by techniques known to one skilled in the art.

In certain embodiments, the NDV used in a combination therapy disclosed herein expresses a mutated F protein. In a specific embodiment, the NDV used in a combination therapy expresses a mutated F protein is highly fusogenic and able to form syncytia. In another specific embodiment, the mutated F protein is incorporated into the virion.

In one embodiment, a genome of a NDV used in a combination therapy disclosed herein is engineered to express a mutated F protein with a mutated cleavage site. In a specific embodiment, the NDV used in a combination therapy disclosed herein is engineered to express a mutated F protein in which the cleavage site of the F protein is mutated to produce a polybasic amino acid sequence, which allows the protein to be cleaved by intracellular proteases, which makes the virus more effective in entering cells and forming syncytia. In another specific embodiment, the NDV used in a combination therapy disclosed herein is engineered to express a mutated F protein in which the cleavage site of the F protein is replaced with a mutated cleavage site containing one or two extra arginine residues, allowing the mutant cleavage site to be activated by ubiquitously expressed proteases of the furin family. Specific examples of NDVs that express such a mutated F protein include, but are not limited to, rNDV/F2aa and rNDV/F3aa. For a description of mutations introduced into a NDV F protein to produce a mutated F protein with a mutated cleavage site, see, e.g., Park et al. (2006) Engineered viral vaccine constructs with dual specificity: avian influenza and Newcastle disease. PNAS USA 103:8203-2808, which is incorporated herein by reference in its entirety. In some embodiments, the NDV used in a combination therapy disclosed herein is engineered to express a mutated F protein with the amino acid mutation L289A (i.e., an L to A mutation at the amino acid position corresponding to L289 of the LaSota F protein). For a description of the L289A mutation, see, e.g., Sergel et al. (2000) A Single Amino Acid Change in the Newcastle Disease Virus Fusion Protein Alters the Requirement for HN Protein in Fusion. Journal of Virology 74 (11): 5101-5107, which is incorporated herein by reference in its entirety. In specific embodiments, the L289A mutated F protein possesses one, two or three arginine residues in the cleavage site. In some embodiments, the NDV used in a combination therapy disclosed herein is the LaSota strain, which has been engineered to express a mutated F protein with the amino acid mutation L289A (i.e., an L to A mutation at the amino acid position corresponding to L289 of the LaSota F protein). In certain embodiments, the NDV used in a combination therapy disclosed herein is the LaSota strain, which has been engineered to express a mutated F protein with the amino acid mutation L289A (i.e., an L to A mutation at the amino acid position corresponding to L289 of the LaSota F protein) and with the LaSota strain F protein cleavage site (GRQGRL (SEQ ID NO: 72)). In some embodiments, the NDV used in a combination therapy disclosed herein is the NDV disclosed in Kim et al., 2017, PLOS ONE 12 (3): c0173965 and Kim et al., 2016, J. of General Virology 97:1297-1303, each of which is incorporated herein by reference in its entirety. In certain embodiments, the mutated F protein is from a different type or strain of NDV than the backbone NDV. In certain embodiments, the mutated F protein is from the same strain of NDV as the backbone NDV. In some embodiments, the mutated F protein is in addition to the backbone NDV F protein. In specific embodiments, the mutated F protein replaces the backbone NDV F protein. In a specific embodiment, the NDV used in a combination therapy disclosed herein comprises a La Sota strain backbone that is engineered to express a mutated F protein with the amino acid mutation L289A. In a specific embodiment, the nucleotide sequence of the La Sota strain genome is as set forth in SEQ ID NO: 50.

In certain embodiments, the NDV used in a combination therapy disclosed herein comprises a mutated F protein with an F protein cleavage site of NDV LaSota strain or glycoprotein B of cytomegalovirus (CMV). In a specific embodiment, the NDV used in a combination therapy disclosed herein comprises a mutated F protein with an F protein cleavage having one of the following sequence modifications: S116: ¹¹¹H-N-R-T-K-S/F¹¹⁷ (SEQ ID NO: 56); S116K: ¹¹¹H-N-K-T-K-S/F¹¹⁷ (SEQ ID NO: 58); S116m: ¹¹¹H-N-R-M-K-S/F¹¹⁷ (SEQ ID NO: 69); S116KM: ¹¹¹H-N-K-M-S/F-I¹¹⁸ (SEQ ID NO: 70); or R116: ¹¹¹H-N-R-T-K-R/F-I¹¹⁸ (SEQ ID NO: 71), such as described in International Patent Application No. WO 2015/032755. See, e.g., International Patent Application Publication No. WO 2015/032755 for a description of the types of mutated F protein cleavage sites that may be engineered into an NDV F protein, which is incorporated herein by reference in its entirety. In some embodiments, the mutated F protein is in addition to the backbone NDV F protein. In specific embodiments, the mutated F protein replaces the backbone NDV F protein.

In another specific embodiment, the NDV used in a combination therapy disclosed herein is the modified 73T strain described in International Patent Application No. WO 2015/032755, which is incorporated herein by reference in its entirety. In another specific embodiment, the NDV used in a combination therapy disclosed herein is the r73T-R116 virus (r73T strain with F protein cleavage site ¹¹¹H-N-R-T-K-R/F-I¹¹⁸ (SEQ ID NO: 71)) described in International Patent Application No. WO 2015/032755, which is incorporated herein by reference in its entirety. In a further embodiment, the NDV comprises an HN and L intergenic non-coding sequence of 60, 102, 144, 198 or 318 nt in length.

In certain embodiments, the NDV used in a combination therapy disclosed herein is attenuated such that the NDV remains, at least partially, infectious and can replicate in vivo, but only generates low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the NDV genome comprises sequences necessary for infection and replication of the attenuated virus such that progeny is produced and the infection level is subclinical. In a specific embodiment, the NDV is replication competent in human cells.

In certain embodiments, the NDV used in a combination therapy disclosed herein does not comprise an NDV V protein encoding sequence. In other embodiments, the NDV used in a combination therapy disclosed herein expresses a mutated V protein. See, e.g., Elankumaran et al., 2010, J. Virol. 84 (8): 3835-3844, which is incorporated herein by reference, for examples of mutated V proteins. In certain embodiments, a mesogenic or velogenic NDV strain used in a combination therapy disclosed herein expresses a mutated V protein, such as disclosed by Elankumaran et al., 2010, J. Virol. 84 (8): 3835-3844.

In certain embodiments, the NDV used in a combination therapy disclosed herein is an NDV disclosed in U.S. Pat. Nos. 7,442,379, 6,451,323, 6,146,642, U.S. Patent Application Publication No. 2014/0271677 A1, International Patent Application Publication No. WO 2014/0158811, or U.S. Patent Application Publication No. 2016/0015760 A1, each of which is incorporated herein by reference in its entirety. In a specific embodiment, the NDV used in a combination therapy disclosed herein is an NDV described in Section 5.1 of U.S. Patent Application Publication No. 2014/0271677 A1, International Patent Application Publication No. WO 2014/0158811, or U.S. Patent Application Publication No. 2016/0015760 A1, each of which is incorporated herein by reference in its entirety. In specific embodiments, the NDV used in a combination therapy disclosed herein is genetically engineered to encode and express a heterologous peptide or protein. In certain embodiments, the NDV used in a combination therapy disclosed herein is a chimeric NDV known to one of skill in the art, or a chimeric NDV disclosed herein (see, e.g., Section 5.2 and/or Section 6, infra). In certain embodiments, the NDV used in a combination therapy disclosed herein is a chimeric NDV disclosed in U.S. Patent Application Publication Nos. 2012/0058141, 2012/0122185, 2016/0015760 A1, or 2014/0271677 A1, or International Patent Application Publication No. WO 2014/158811, each of which is incorporated herein by reference in its entirety. In specific embodiments, the NDV used in a combination therapy disclosed herein is a chimeric NDV comprising a genome engineered to express a cytokine, such as, e.g., IL-12.

### 5.2 Chimeric Newcastle Disease Virus

In one aspect, described herein are chimeric NDVs, comprising a packaged genome comprising a transgene encoding IL-12 or an IL-12 derivative (see, e.g., Section 5.2.1). In other words, the NDV serves as the “backbone” that is engineered to encode IL-12 or an IL-12 derivative, which is expressed in cells infected with the virus. In a specific embodiment, the chimeric NDV comprises IL-12 or a derivative thereof. Any NDV type or strain may serve as the backbone of a chimeric NDV described herein, including, but not limited to, naturally-occurring strains, variants or mutants, mutagenized viruses, reassortants and/or genetically engineered viruses. In a specific embodiment, the NDV that serves as the backbone for genetic engineering of the chimeric NDV is an NDV described in Section 5.1, supra. In a specific embodiment, the chimeric NDV preferentially replicates in cancer cells as compared to non-cancer cells. In a specific embodiment, the chimeric NDV is attenuated but remains, at least, partially infectious and can replicate in vivo, but is non-pathogenic and only generates low titers of NDV progeny, resulting in subclinical levels of infection. In a specific embodiment, the chimeric NDV genome comprises sequences necessary for infection and replication of the virus such that progeny is produced and the infection level is subclinical. Techniques for attenuating NDV are known in the art, such as, e.g., mutations or substitutions within the genome and modification or deletion of the NDV V protein, and can be used to attenuate a chimeric NDV described herein. In a specific embodiment, the chimeric NDV is replication competent in human cells.

In a specific aspect, described herein are chimeric NDVs, comprising a packaged genome comprising a transgene encoding IL-12 or a derivative thereof, which may be expressed in cells infected with the virus. In another specific embodiment, described herein are chimeric NDVs, comprising a packaged genome comprising a transgene encoding IL-12 or a derivative thereof and a nucleotide sequence encoding a mutated F protein. In a particular embodiment, the mutated F protein is highly fusogenic. In a specific embodiment, the mutated F protein has a mutant cleavage site (such as described herein). In some embodiments, the mutated F protein comprises the amino acid mutation L289A (i.e., an L to A substitution at the amino acid position corresponding to L289 of the LaSota F protein). In some embodiments, the chimeric NDV comprise a packaged genome comprising a nucleotide sequence encoding a mutated F protein with the amino acid mutation L289A (i.e., an L to A substitution at the amino acid position corresponding to L289 of the LaSota F protein). In certain embodiments, the mutated F protein is from a different type or strain of NDV than the backbone NDV. In other embodiments, the mutated F protein is from the same type or strain of NDV as the backbone NDV. In specific embodiments, the L289A mutated F protein possesses one, two or three arginine residues in the cleavage site. In some embodiments, the mutated F protein is in addition to the backbone NDV F protein. In specific embodiments, the mutated F protein replaces the backbone NDV F protein. In specific embodiments, the mutated F protein is incorporated into the virion. In a specific embodiment, the chimeric NDV comprises a packaged genome comprising a nucleotide sequence encoding a mutated F protein and a nucleotide sequence comprising a transgene encoding IL-12 (e.g., human IL-12), wherein the NDV that serves as the backbone of the chimeric NDV is lentogenic. In a specific embodiment, the chimeric NDV comprises a packaged genome comprising a nucleotide sequence encoding a mutated F protein and a nucleotide sequence comprising a transgene encoding IL-12 (e.g., human IL-12), wherein the nucleotide sequence encoding the mutated F protein replaces the F protein of the genome of the NDV that serves as the backbone of the chimeric NDV. In a specific embodiment, the chimeric NDV comprises a packaged genome comprising a nucleotide sequence encoding a mutated F protein and a nucleotide sequence comprising a transgene encoding IL-12 (e.g., human IL-12), wherein the NDV that serves as the backbone of the chimeric NDV is La Sota strain. In another specific embodiment, the chimeric NDV comprises a packaged genome comprising a nucleotide sequence encoding a mutated NDV F protein and a nucleotide sequence comprising a transgene encoding IL-12 (e.g., human IL-12), wherein the NDV that serves as the backbone of the chimeric NDV is La Sota strain, wherein the nucleotide sequence encoding the mutated F protein replaces the F protein of the genome of the NDV that serves as the backbone of the chimeric NDV, and wherein the mutated NDV F protein has the amino acid mutation L289A (i.e., an L to A substitution at the amino acid position corresponding to L289 of the LaSota F protein). In another specific embodiment, the chimeric NDV comprises a packaged genome comprising a nucleotide sequence encoding a mutated NDV F protein and a nucleotide sequence comprising a transgene encoding IL-12 (e.g., human IL-12), wherein the NDV that serves as the backbone of the chimeric NDV is La Sota strain, wherein the nucleotide sequence encoding the mutated F protein replaces the F protein of the genome of the NDV that serves as the backbone of the chimeric NDV, and wherein the mutated NDV F protein has the amino acid mutation L289A (i.e., an L to A substitution at the amino acid position corresponding to L289 of the LaSota F protein) and the LaSota strain F protein cleavage site (GRQGRL (SEQ ID NO: 72)).

In some embodiments, described herein are chimeric NDVs, comprising a packaged genome comprising a transgene encoding IL-12 or a derivative thereof, wherein the NDV that serves as the backbone of the chimeric NDV is an NDV disclosed in Kim et al., 2017, PLOS ONE 12 (3): e0173965 and Kim et al., 2016, J. of General Virology 97:1297-1303, each of which is incorporated herein by reference in its entirety.

In some embodiments, described herein are chimeric NDVs, comprising a packaged genome comprising a transgene encoding IL-12 or a derivative thereof, wherein the NDV that serves as the backbone of the chimeric NDV comprises a genome having the sequence set forth in SEQ ID NO: 50. In specific embodiments, described herein is a chimeric NDV comprising a packaged genome, wherein the packaged genome comprises or consists of the nucleotide sequence of SEQ ID NO:50 and a transgene encoding IL-12 (e.g., human IL-12). In particular embodiments, the transgene is inserted between the transcription units for two NDV genes (e.g., the P and M transcription units).

In some embodiments, described herein are chimeric NDV comprising a packaged genome comprising (i) a transgene encoding IL-12 or a derivative thereof and (ii) a nucleotide sequence encoding a mutated NDV V protein-encoding sequence, such as disclosed by Elankumaran et al., 2010, J. Virol. 84 (8): 3835-3844. In other embodiments, a chimeric NDV comprising a packaged genome comprising a transgene encoding IL-12 or a derivative thereof does not comprise an NDV V protein-encoding sequence. In certain embodiments, the parental backbone of the chimeric NDV is a mesogenic or velogenic NDV strain that is engineered to encode a mutated V protein, such as disclosed by Elankumaran et al., 2010, J. Virol. 84 (8): 3835-3844.

In certain embodiments, provided herein are chimeric NDV comprising a packaged genome comprising a transgene encoding IL-12 or a derivative thereof and a nucleotide sequence encoding a mutated F protein, wherein the mutated F protein has an F protein cleavage site of NDV LaSota strain or glycoprotein B of cytomegalovirus (CMV). In a specific embodiment, provided herein are chimeric NDV comprising a packaged genome comprising a transgene encoding IL-12 or a derivative thereof and a nucleotide sequence encoding a mutated F protein, wherein the mutated F protein has an F protein cleavage having one of the following sequences: S116: ¹¹¹H-N-R-T-K-S/F¹¹⁷ (SEQ ID NO: 56); S116K: ¹¹¹H-N-K-T-K-S/F¹¹⁷ (SEQ ID NO: 58); S116m: ¹¹¹H-N-R-M-K-S/F¹¹⁷ (SEQ ID NO: 69); S116KM: ¹¹¹H-N-K-M-S/F-I¹¹⁸ (SEQ ID NO: 70); or R116: ¹¹¹H-N-R-T-K-R/F-I¹¹⁸ (SEQ ID NO: 71), such as described in International Patent Application No. WO 2015/032755. See, e.g., International Patent Application Publication No. WO 2015/032755 for a description of the types of mutated F protein cleavage sites that may be engineered into an NDV F protein, which is incorporated herein by reference in its entirety. In some embodiments, the mutated F protein is in addition to the backbone NDV F protein. In specific embodiments, the mutated F protein replaces the backbone NDV F protein.

In certain embodiments, provided herein are chimeric NDV comprising a packaged genome comprising a transgene encoding IL-12 or a derivative thereof, wherein the NDV that serves as the backbone of the chimeric NDV is the modified 73T strain described in International Patent Application No. WO 2015/032755, which is incorporated herein by reference in its entirety. In another specific embodiment, provided herein are chimeric NDV comprising a packaged genome comprising a transgene encoding IL-12 or a derivative thereof, wherein the NDV that serves as the backbone of the chimeric NDV is the r73T-R116 virus (r73T strain with F protein cleavage site ¹¹¹H-N-R-T-K-R/F-I¹¹⁸ (SEQ ID NO: 71)) described in International Patent Application No. WO 2015/032755, which is incorporated herein by reference in its entirety. In a further embodiment, the chimeric NDV comprises an HN and L intergenic non-coding sequence of 60, 102, 144, 198 or 318 nucleotide in length.

In a specific embodiment, the chimeric NDV is a chimeric NDV described in Section 6, infra. In a preferred embodiment, the chimeric NDV is the NDV-huIL-12 described in Section 6, infra. In another preferred embodiment, the chimeric NDV comprises a genome having the sequence set forth in SEQ ID NO: 51. In another embodiment, the chimeric NDV comprises a genome having the sequence set forth in SEQ ID NO: 52. In another preferred embodiment, the chimeric NDV comprises a genome having the sequence set forth in SEQ ID NO: 60.

In a specific embodiment, the chimeric NDV comprises a packaged genome, said packaged genome comprising a transgene encoding a human IL-12, wherein the IL-12 comprises the amino acid sequence set forth in SEQ ID NO:39. In a specific embodiment, the transgene comprises the nucleotide sequence set forth in SEQ ID NO:61.

In a specific embodiment, the chimeric NDV comprises a packaged genome, said packaged genome comprising a transgene encoding a human IL-12, wherein the IL-12 comprises the amino acid sequence set forth in SEQ ID NO:22, wherein said IL-12 comprises a signal peptide. In a specific embodiment, the transgene comprises the nucleotide sequence set forth in SEQ ID NO:26.

In a specific embodiment, the chimeric NDV comprises a packaged genome, said packaged genome comprising a transgene encoding a human IL-12, wherein the IL-12 comprises the amino acid sequence set forth in SEQ ID NO:43. In a specific embodiment, the transgene comprises the nucleotide sequence set forth in SEQ ID NO:63. In a specific embodiment, the transgene comprises the nucleotide sequence set forth in SEQ ID NO:68.

In a specific embodiment, the chimeric NDV comprises a packaged genome, said packaged genome comprising a transgene encoding a human IL-12, wherein the IL-12 comprises the amino acid sequence set forth in SEQ ID NO:42, wherein said IL-12 comprises a signal peptide. In a specific embodiment, the transgene comprises the nucleotide sequence set forth in SEQ ID NO:53. In a specific embodiment, the transgene comprises the nucleotide sequence set forth in SEQ ID NO:66.

In a specific embodiment, a chimeric NDV comprising a packaged genome comprising a transgene encoding IL-12 or a derivative thereof induces the expression of IL-12p70, IFN-γ expression, or both IL-12p70 and IFN-γ in an assay described herein (e.g., an assay described in Section 6, infra). In another specific embodiment, treatment of a tumor sample with a chimeric NDV comprising a packaged genome comprising a transgene encoding IL-12 or a derivative thereof results in an increase in the gene expression profile (GEP) relative to the GEP score of the tumor sample prior to treatment with the chimeric NDV. See, for example, Example 6.3, infra, regarding the GEP score. In another specific embodiment, treatment of a tumor sample, such as described in Section 6, infra, with a chimeric NDV comprising a packaged genome comprising a transgene encoding IL-12 increases the GEP score of the tumor sample relative to the GEP score of the tumor sample prior to treatment with the chimeric NDV, such as described in Section 6, infra. In one embodiment, the tumor sample of a subject has a Gene Expression Profile (GEP) score of the 18-gene signature of Table 15 at less than-0.318. In another embodiment, the tumor of the subject has a Gene Expression Profile (GEP) score of the 18-gene signature of Table 15 at greater than-0.318. Without being bound by any theory, it is believed that an increase in GEP score will result in the tumor being more likely to be responsive to treatment with an anti-PD-1 antibody (e.g., an anti-PD-1 blocking antibody), such as pembrolizumab. Thus, in a specific embodiment, the administration of a chimeric NDV comprising a packaged genome comprising a transgene encoding IL-12 to a patient with cancer refractory, relapsed or unresponsive to treatment with an anti-PD-1 antibody (e.g., an anti-PD-1 blocking antibody, such as pembrolizumab or another antibody described in Section 5.5) may result in the patient becoming responsive to treatment with the anti-PD-1 antibody (e.g., an anti-PD-1 blocking antibody, such as pembrolizumab or another antibody described in Section 5.5). In particular, administration of a a chimeric NDV comprising a packaged genome comprising a transgene encoding IL-12 to a patient with cancer refractory, relapsed or unresponsive to treatment with pembrolizumab (KEYTRUDA®, Merck & Co., Inc. Kenilworth, NJ) may result in the patient becoming responsive to treatment with pembrolizumab. Therefore, in one embodiment, provided herein is a method of increasing response to anti-PD-1 therapy in a subject with cancer.

In another embodiment, described herein are chimeric NDVs, comprising a packaged genome comprising (i) a transgene encoding IL-12 or a derivative thereof, and (ii) a transgene encoding a heterologous interferon antagonist. See, e.g., U.S. Patent Application Publication No. 2012-0058141, which is incorporated herein by reference, for examples of chimeric NDV engineered to express heterologous interferon antagonists.

Interferon antagonists may be identified using any technique known to one of skill in the art, including, e.g., the techniques described in U.S. Pat. Nos. 6,635,416; 7,060,430; and 7,442,527; each of which is incorporated herein by reference in their entirety. In a specific embodiment, the heterologous interferon antagonist is a viral protein. Such viral proteins may be obtained or derived from any virus and the virus may infect any species (e.g., the virus may infect humans or non-human mammals). Exemplary heterologous interferon antagonists include, without limitation, Nipah virus W protein, Nipah V protein, Ebola virus VP35 protein, vaccinia virus E3L protein, influenza virus NS1 protein, respiratory syncytial virus (RSV) NS2 protein, herpes simplex virus (HSV) type 1 ICP34.5 protein, Hepatitis C virus NS3-4 protease, dominant-negative cellular proteins that block the induction or response to innate immunity (e.g., STAT1, MyD88, IKK and TBK), and cellular regulators of the innate immune response (e.g., SOCS proteins, PIAS proteins, CYLD proteins, IkB protein, Atg5 protein, Pin1 protein, IRAK-M protein, and UBP43). See, e.g., U.S. patent application publication No. 2012-0058141, which is incorporated herein by reference in its entirety, for additional information regarding heterologous interferon antagonist.

Any NDV type or strain may serve as the backbone for the chimeric NDV comprising a packaged genome that is engineered to encode IL-12 (e.g., human IL-12) and, in certain embodiments, a heterologous interferon antagonist and/or mutated F protein, including, but not limited to, naturally-occurring strains, variants or mutants, mutagenized viruses, reassortants and/or genetically engineered viruses. In a specific embodiment, the NDV that serves as the backbone for genetic engineering of the chimeric NDV is an NDV described in Section 5.1. In a specific embodiment, the NDV that serves as the backbone for genetic engineering of the chimeric NDV is a naturally-occurring strain. In certain embodiments, the NDV that serves as the backbone for genetic engineering of the chimeric NDV is a lytic strain. In other embodiments, the NDV that serves as the backbone for genetic engineering of the chimeric NDV is a non-lytic strain. In certain embodiments, the NDV that serves as the backbone for genetic engineering of the chimeric NDV is a lentogenic strain. In some embodiments, the NDV that serves as the backbone for genetic engineering of the chimeric NDV is a mesogenic strain. In other embodiments, the NDV that serves as the backbone for genetic engineering of the chimeric NDV is a velogenic strain. Specific examples of NDV strains include, but are not limited to, the 73-T strain, NDV HUJ strain, Ulster strain (see, e.g., GenBank No. U25837), MTH-68 strain, Italien strain (see, e.g., GenBank No. EU293914), Hickman strain (see, e.g., Genbank No. AF309418), PV701 strain, Hitchner B1 strain (see, e.g., GenBank No. AF309418 or NC_002617), La Sota strain (see, e.g., GenBank Nos. AY845400 and JF950510.1 and GI No. 56799463), YG97 strain (see, e.g., GenBank Nos. AY351959 or AY390310), MET95 strain (see, e.g., GenBank No. AY143159), Roakin strain (see, e.g., GenBank No. AF124443), and F48E9 strain (see, e.g., GenBank Nos. AF163440 and U25837). In a specific embodiment, the NDV that serves as the backbone for genetic engineering of the chimeric NDV is the Hitchner B1 strain. In another specific embodiment, the NDV that serves as the backbone for genetic engineering of the chimeric NDV is a B1 strain as identified by GenBank No. AF309418 or NC_002617. In another specific embodiment, the NDV that serves as the backbone for genetic engineering of the chimeric NDV is the NDV identified by ATCC No. VR2239. In another specific embodiment, the NDV that serves as the backbone for genetic engineering is the La Sota strain.

In certain embodiments, attenuation, or further attenuation, of the chimeric NDV is desired such that the chimeric NDV remains, at least partially, infectious and can replicate in vivo, but only generates low titers resulting in subclinical levels of infection that are non-pathogenic (see, e.g., Khattar et al., 2009, J. Virol. 83:7779-7782). In a specific embodiment, the chimeric NDV is attenuated by deletion of the V protein. Such attenuated chimeric NDVs may be especially suited for embodiments wherein the virus is administered to a subject in order to act as an immunogen, e.g., a live vaccine. The viruses may be attenuated by any method known in the art.

In a specific embodiment of the chimeric NDV comprising a packaged genome comprising a transgene encoding IL-12 or a derivative thereof, the packaged genome also comprises one or more other transgenes, such as, e.g., an antagonist of an inhibitory receptor of an immune cell, an agonist of a co-stimulatory receptor of an immune cell, an additional cytokine, a tumor antigen, a pro-apoptotic molecule, an anti-apoptotic molecule, a suicide gene, or a heterologous interferon antagonist. Nonlimiting examples of inhibitory receptors of an immune cell are provided in Section 5.7.6.1. Nonlimiting examples of co-stimulatory receptors of an immune cell are provided in Section 5.7.6.1. Nonlimiting examples of cytokines include IL-2 (e.g., Genbank Accession No. NM_000586.3 and GI No. 125661059), IL-7 (e.g., Genbank Accession Nos. NM_000880.3, NM_001199886.1, NM_001199887.1 and NM 001199888.1 and GI Nos. 315467865, 315467866, 315467868, and 315467870), IL-9 (e.g., Genbank Accession No. NM 000590.1 and GI No. 10834979), IL-15 (e.g., Genbank Accession Nos. NM_172175.2 and NM_000585.4 and GI Nos. 323098328 and 323098327), IL-17 (e.g., Genbank Accession No. NM_002190.2 and GI No. 27477085), IL-21 (e.g., Genbank Accession Nos. NM_021803.3 and NM_001207006.2 and GI Nos. 365733583 and 365733582), IL-22 (e.g., Genbank Accession No. NM_020525.4 and GI No. 41393566), IFN-gamma (e.g., Genbank Accession No. NM_000619.2 and GI No. 56786137), GM-CSF (e.g., Genbank Accession No. M11220_1 and GI No. 183363), and TNF-alpha (e.g., Genbank Accession No. NM_000594.3). Nonlimiting examples of tumor antigens include MAGE-1, MAGE-3, BAGE, GAGE-1, GAGE-2, N-acetylglucosaminyltransferase-V, p=15, gp100, MART-1/MelanA, TRP-1 (gp75), Tyrosinase, cyclin-dependent kinase 4, β-catenin, MUM-1, CDK4, HER-2/neu, human papillomavirus-E6, human papillomavirus E7, CD20, carcinoembryonic antigen (CEA), epidermal growth factor receptor, MUC-1, caspase-8, CD5, mucin-1, Lewisx, CA-125, p185HER2, IL-2R, Fap-α, tenascin, antigens associated with a metalloproteinase, and CAMPATH-1. Other examples of tumor antigens include, but are not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, CEA, polymorphic epithelial mucin antigen, milk fat globule antigen, colorectal tumor-associated antigens (such as: CEA, TAG-72, CO17-1A, GICA 19-9, CTA-1 and LEA), Burkitt's lymphoma antigen-38.13, CD19, B-lymphoma antigen-CD20, CD33, melanoma specific antigens (such as ganglioside GD2, ganglioside GD3, ganglioside GM2, ganglioside GM3), tumor-specific transplantation type of cell-surface antigen (TSTA) (such as virally-induced tumor antigens including T-antigen DNA tumor viruses and Envelope antigens of RNA tumor viruses), oncofetal antigen-alpha-fetoprotein such as CEA of colon, bladder tumor oncofetal antigen, differentiation antigen (such as human lung carcinoma antigen L6 and L20), antigens of fibrosarcoma, leukemia T cell antigen-Gp37, neoglycoprotein, sphingolipids, breast cancer antigens (such as EGFR (Epidermal growth factor receptor), HER2 antigen (^{p185}HER2) and HER2 neu epitope), polymorphic epithelial mucin (PEM), malignant human lymphocyte antigen-APO-1, differentiation antigen (such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D156-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E1 series (blood group B) found in pancreatic cancer, FC10.2 found in embryonal carcinoma cells, gastric adenocarcinoma antigen, CO-514 (blood group Lea) found in Adenocarcinoma, NS-10 found in adenocarcinomas, CO-43 (blood group Leb), G49 found in EGF receptor of A431 cells, MH2 (blood group ALeb/Lcy) found in colonic adenocarcinoma, 19.9 found in colon cancer, gastric cancer mucins, T5A7 found in myeloid cells, R24 found in melanoma, 4.2, GD3, D1.1, OFA-1, GM2, OFA-2, GD2, and M1:22:25:8 found in embryonal carcinoma cells, and SSEA-3 and SSEA-4 found in 4 to 8-cell stage embryos), T cell receptor derived peptide from a cutaneous T cell lymphoma, C-reactive protein (CRP), cancer antigen-50 (CA-50), cancer antigen 15-3 (CA15-3) associated with breast cancer, cancer antigen-19 (CA-19) and cancer antigen-242 associated with gastrointestinal cancers, carcinoma associated antigen (CAA), chromogranin A, epithelial mucin antigen (MC5), human epithelium specific antigen (EIA), Lewis (a) antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdr1 gene product), multidrug-resistance-related antigen, p170, multidrug-resistance-related antigen, prostate specific antigen (PSA), CD56, and NCAM. Nonlimiting examples of pro-apoptotic molecules include Bax, Bak, Bad, BID, Bcl-xS, Bim, Noxa, Puma, AIF, FasL, and TRAIL. Nonlimiting examples of anti-apoptotic molecules include Bcl-2, Bcl-xL, Mcl-1, and XIAP. A nonlimiting example of a suicide gene includes thymidine kinase.

In certain embodiments, a chimeric NDV described herein comprises a packaged genome comprising a transgene encoding IL-12 or a derivative thereof and one, two, three, or more, or all of the following transgenes: (1) an agonist of a co-stimulatory signal of an immune cell; (2) an antagonist of an inhibitory signal of an immune cell; (3) a cytokine; (4) a tumor antigen; (5) a heterologous interferon antagonist; (6) a pro-apoptotic molecule; (7) an anti-apoptotic molecule; and/or (8) a suicide gene. In addition, the packaged genome may comprise a nucleotide sequence encoding a mutated F protein (such as, e.g., a mutated F protein described herein). In specific embodiments, in addition to the packaged genome comprising a transgene encoding IL-12 or a derivative thereof, and in certain embodiments, the packaged genome comprising a nucleotide sequence encoding a mutated F protein and/or a nucleotide sequence encoding a heterologous interferon antagonist, the packaged genome of a chimeric NDV comprises a transgene encoding a suicide gene (e.g., thymidine kinase) or another molecule that inhibits NDV replication or function (a gene that makes NDV sensitive to an antibiotic or an anti-viral agent). In some embodiments, in addition to the packaged genome comprising a transgene encoding IL-12 or a derivative thereof, and in certain embodiments, the packaged genome comprising a nucleotide sequence encoding a mutated F protein and/or a nucleotide sequence encoding a heterologous interferon antagonist, the packaged genome of a chimeric NDV comprise nucleotide sequence encoding tissue-specific microRNA (miRNA) target sites (e.g., sites targeted by miR-21, miR-184, miR-133a/133b, miR-137, and/or miR-193a microRNAs). In some embodiments, the packaged genome does not comprise a nucleotide sequence encoding a miRNA target site(s).

In certain embodiments, a chimeric NDV described herein comprises a packaged genome comprising a transgene encoding IL-12 or a derivative thereof, wherein the packaged genome does not encode a heterologous interferon antagonist.

In certain embodiments, the tropism of the chimeric NDV is altered. In a specific embodiment, the tropism of the virus is altered by modification of the F protein cleavage site to be recognized by tissue-specific or tumor-specific proteases such as matrix metalloproteases (MMP) and urokinase. In other embodiments, tropism of the virus is altered by introduction of tissue-specific miRNA target sites. In certain embodiments, NDV HN protein is mutated to recognize tumor-specific receptor.

In certain embodiments, a chimeric NDV described herein comprises a packaged genome comprising a transgene encoding IL-12 or a derivative therein, wherein the packaged genome of a chimeric NDV described herein does not comprise an additional transgene(s). In certain embodiments, the packaged genome of a chimeric NDV described herein does not comprise a transgene(s) encoding a heterologous interferon antagonist(s). In certain embodiments, the packaged genome of a chimeric NDV described herein does not comprise a transgene(s) encoding one, two, or more, or all of the following: (1) a transgene(s) encoding one or more cytokines other than IL-12; (2) a transgene(s) encoding one or more tumor antigens; (3) a transgene(s) encoding one or more anti-apoptotic molecule(s); (4) a transgene(s) encoding a suicide gene; (5) a transgene(s) encoding an agonist(s) of a co-stimulatory signal of an immune cell; (6) a transgene(s) encoding one or more antagonist(s) of an inhibitory signal of an immune cell; or (7) a transgene(s) encoding one or more pro-apoptotic molecule(s).

In certain embodiment, the genome of the chimeric NDV does not comprise a heterologous sequence encoding a heterologous protein other than IL-12 or a derivative thereof (e.g., human IL-12). In certain embodiments, a chimeric NDV described herein comprises a packaged genome, wherein the genome comprises the genes found in NDV and a transgene encoding IL-12 or a derivative thereof. In some embodiments, a chimeric NDV described herein comprises a packaged genome, wherein the genome comprises the genes found in NDV and a transgene encoding IL-12 or a derivative thereof but does not include any other transgenes.

In particular embodiments, the genome of the chimeric NDV does not comprises a heterologous sequence encoding a heterologous protein other than IL-12 or a derivative thereof and a mutated NDV F protein, wherein the mutated NDV F protein replaces the naturally occurring NDV F protein. In certain embodiments, a chimeric NDV described herein comprises a packaged genome, wherein the genome comprises: (1) the genes found in NDV other than an NDV F protein; (2) a transgene encoding IL-12 or a derivative thereof (e.g., human IL-12), and (3) a mutated NDV F protein, such as described herein, which replaces the NDV F protein. In some embodiments, a chimeric NDV described herein comprises a packaged genome, wherein the genome comprises (1) the genes found in NDV other than an NDV F protein; (2) a transgene encoding IL-12 or a derivative thereof (e.g., human IL-12), and (3) a mutated NDV F protein, such as described herein, which replaces the NDV F protein, but does not include any other transgenes.

In certain embodiments, one or more of the following are expressed by a chimeric NDV in cells as a chimeric protein or fusion protein: (1) IL-12 or a derivative thereof; (2) a heterologous interferon antagonist; and/or (3) a mutated F protein. In specific embodiments, IL-12 or a derivative thereof is expressed by a chimeric NDV in cells as a chimeric protein or fusion protein. In specific embodiments, a mutated F protein is expressed by a chimeric NDV as a chimeric protein or a fusion protein. In specific embodiments, the chimeric protein or fusion protein comprises the transmembrane and cytoplasmic domains or fragments thereof of the NDV F or NDV HN protein and an extracellular domain that comprises one of the molecules referenced in the previous sentence. See U.S. Patent Application No. 2012-0122185 for a description of such chimeric proteins or fusion proteins, and International Application Publication No. WO 2007/064802, each of which is incorporated herein by reference.

In embodiments herein, the transgene encoding IL-12 or a derivative thereof may be inserted into the genome of the backbone NDV between two transcription units. In a specific embodiment, the transgene encoding IL-12 or a derivative thereof is inserted into the genome of the backbone NDV between the M and P transcription units or between the HN and L transcription units. In accordance with other embodiments herein, one or more other transgenes or nucleotide sequences described herein, such as those encoding a heterologous interferon antagonist and/or a mutated F protein may be inserted into the genome of the backbone NDV between two or more transcription units (e.g., between the M and P transcription units or between the HN and L transcription units).

In some embodiments, the chimeric NDV is an NDV described in Section 5.2 of U.S. Patent Application Publication No. 2014/0271677 A1 or 2016/0015760 A1, or International Patent Application Publication No. WO 2014/158811, each of which is incorporated herein by reference in its entirety.

### 5.2.1 IL-12

In one aspect, provided herein is a chimeric NDV comprising a packaged genome, wherein the packaged genome comprises a transgene encoding IL-12 or a derivative thereof (e.g., human IL-12). The chimeric NDV may be used alone or in combination with one or more other therapies, such as an antagonist of PD-1 or a ligand thereof, to treat cancer.

In another aspect, presented herein are methods for treating cancer utilizing a chimeric NDV or a composition comprising the chimeric NDV in combination with an antagonist of PD-1 or a ligand thereof or a composition comprising such an antagonist, wherein the chimeric NDV comprises a packaged genome comprising a transgene encoding interleukin-12 (“IL-12”) (e.g., the p35 and p40 subunits of IL-12) or a derivative thereof. In specific embodiments, the chimeric NDV comprises a packaged genome comprising a first transgene encoding the IL-12 p35 subunit or a derivative thereof and a second transgene encoding the IL-12 p40 subunit or a derivative thereof. In a specific embodiment, the IL-12 or derivative thereof is expressed by cells infected with the chimeric NDV. In specific embodiments, the antagonist is a PD-1 blocking antibody (e.g., nivolumab or pembrolizumab). In some embodiments, the antagonist is a PD-L1 blocking antibody (e.g., avelumab).

In another aspect, presented herein are a chimeric NDV or a composition comprising the chimeric NDV for use in a method for treating cancer in a subject (e.g., a human subject), wherein the chimeric NDV comprises a packaged genome comprising a transgene encoding IL-12 (e.g., the p35 and p40 subunits of IL-12) or a derivative thereof, and wherein the method further comprises administering an antagonist of PD-1 or a ligand thereof or a composition comprising such an antagonist. In a specific embodiment, the IL-12 or derivative thereof is expressed by cells infected with the chimeric NDV. In specific embodiments, the antagonist is a PD-1 blocking antibody (e.g., nivolumab or pembrolizumab). In some embodiments, the antagonist is a PD-L1 blocking antibody (e.g., avelumab).

The IL-12 or derivative thereof encoded by a transgene in a packaged genome of a chimeric NDV described herein may be any IL-12 known to those of skill in the art. In certain embodiments, the IL-12 or a derivative thereof is human, dog, cat, horse, pig, or cow IL-12 or a derivative thereof. In a specific embodiment, the IL-12 or a derivative thereof is human IL-12 or a derivative thereof. A typical IL-12 consists of a heterodimer encoded by two separate genes, IL-12A (the p35 subunit) and IL-12B (the p40 subunit), known to those of skill in the art. GenBank™ accession number NM_000882.3 (GI number 325974478) provides an exemplary human IL-12A nucleic acid sequence. GenBank™ accession number NM_002187.2 (GI number 24497437) provides an exemplary human IL-12B nucleic acid sequence. GenBank™ accession number NP_000873.2 (GI number 24430219) provides an exemplary human IL-12A (the p35 subunit) amino acid sequence. GenBank™ accession number NP_002178.2 (GI number 24497438) provides an exemplary human IL-12B (the p40 subunit) amino acid sequence. In certain embodiments, the IL-12 or a derivative thereof encoded by a packaged genome of a chimeric NDV described herein consists of a single polypeptide chain, comprising the IL-12 p35 subunit (also referred to as “IL-12A”) or a derivative thereof and the IL-12 p40 subunit (also referred to as “IL-12B”) or a derivative thereof, optionally separated by a linker sequence. In certain embodiments, the IL-12 or a derivative thereof encoded by a packaged genome of a chimeric NDV described herein consists of two polypeptide chains: (i) a first polypeptide comprising the IL-12 p35 subunit or a derivative thereof, and (ii) a second polypeptide comprising the IL-12 p40 subunit or a derivative thereof. In certain embodiments, the transgene encoding the IL-12 or a derivative thereof comprises a nucleotide sequence encoding the IL-12 p35 subunit and a nucleotide sequence encoding the IL-12 p40 subunit, wherein the nucleotide sequence encoding the IL-12 p35 subunit and the nucleotide sequence encoding the IL-12 p40 subunit are separated by an internal ribosome entry site. SEQ ID NOs: 29, 55, and 65 provide exemplary nucleotide sequences encoding the IL-12 p35 subunit. SEQ ID NOs: 27, 54, 57, 59, and 64 provide exemplary nucleotide sequences encoding the IL-12 p40 subunit. In a specific embodiment, an IL-12 comprises the p35 and p40 subunit sequences set forth in SEQ ID NOs: 41 and 38, respectively. In another specific embodiment, an IL-12 comprises the p35 and p40 subunit sequences set forth in SEQ ID NOs: 25 and 38. In a preferred embodiment, an IL-12 comprises the p35 and p40 subunit sequences provided in Section 6, e.g., SEQ ID NOs: 25 and 23, respectively, or SEQ ID NOs: 41 and 40, respectively. In a specific embodiment, the IL-12 encoded by a packaged genome of a chimeric NDV described herein consists of a single polypeptide chain comprising the amino acid sequence set forth in SEQ ID NO: 43. In a specific embodiment, the IL-12 encoded by a packaged genome of a chimeric NDV described herein consists of a single polypeptide chain comprising the amino acid sequence set forth in SEQ ID NO: 42. In a specific embodiment, the IL-12 encoded by a packaged genome of a chimeric NDV described herein consists of a single polypeptide chain comprising the amino acid sequence set forth in SEQ ID NO: 39. In a preferred embodiment, the IL-12 encoded by a packaged genome of a chimeric NDV described herein consists of a single polypeptide chain comprising an amino acid sequence provided in Section 6, e.g., SEQ ID NO: 22. In a specific embodiment, the nucleotide sequence of the transgene is as set forth in SEQ ID NO: 26, 53, 61, 63, 66, or 68. In a specific embodiment, the nucleotide sequence of the transgene comprises the nucleotide sequence set forth in SEQ ID NO: 26, 53, 61, 63, 66, or 68. In a specific embodiment, the IL-12 p35 subunit and IL-12 p40 subunit or derivative(s) thereof are directly fused to each other. In specific embodiments, a polypeptide comprising the IL-12 p35 subunit and IL-12 p40 subunit or derivative(s) thereof directly fused to each other is functional (e.g., capable of specifically binding to the IL-12 receptor and inducing IL-12-mediated signal transduction and/or IL-12-mediated immune function). In a specific embodiment, the IL-12 p35 subunit and IL-12 p40 subunit or derivative(s) thereof are indirectly fused to each other using one or more linkers. Linkers suitable for preparing the IL-12 p35 subunit/p40 subunit fusion protein may comprise one or more amino acids (e.g., a peptide). In specific embodiments, a polypeptide comprising the IL-12 p35 subunit and IL-12 p40 subunit or derivative(s) thereof indirectly fused to each other using an amino acid linker (e.g., a peptide linker) is functional (e.g., capable of specifically binding to the IL-12 receptor and inducing IL-12-mediated signal transduction and/or IL-12-mediated immune function). In a specific embodiment, the linker is long enough to preserve the ability of the IL-12 p35 subunit and IL-12 p40 subunit or derivative(s) thereof to form a functional IL-12 heterodimer complex, which is capable of binding to the IL-12 receptor and inducing IL-12-mediated signal transduction. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is an amino acid sequence (e.g., a peptide) that is between 5 and 20 or 5 and 15 amino acids in length. In certain embodiments, an IL-12 or a derivative thereof encoded by a transgene in a packaged genome of a chimeric NDV described herein consists of more than one polypeptide chain in quaternary association, e.g., a polypeptide chain comprising the IL-12 p35 subunit or a derivative thereof in quaternary association with a polypeptide chain comprising the IL-12 p40 subunit or a derivative thereof. In certain embodiments, the linker is the amino acid sequence set forth in SEQ ID NO: 24. In certain embodiments, the linker is the amino acid sequence set forth in SEQ ID NO: 46. In certain embodiments, the linker is the amino acid sequence set forth in SEQ ID NO: 47. In certain embodiments, the linker is the amino acid sequence set forth in SEQ ID NO: 48. In certain embodiments, the linker is the amino acid sequence set forth in SEQ ID NO: 49. In certain embodiments, the linker is an elastin-like polypeptide sequence. In certain embodiments, the elastin-like polypeptide sequence comprises the amino acid sequence VPGXG (SEQ ID NO:44), wherein X is any amino acid except proline. In certain embodiments, the elastin-like polypeptide sequence comprises the amino acid sequence VPGXGVPGXG (SEQ ID NO:45), wherein X is any amino acid except proline. In certain embodiments, the linker may be a linker described in U.S. Pat. No. 5,891,680, which is incorporated by reference herein in its entirety.

In a specific embodiment, IL-12 encoded by a transgene in a packaged genome of a chimeric NDV described herein comprises the amino acid sequence of a sequence set forth in Table 7. In another specific embodiment, IL-12 encoded by a transgene in a packaged genome of a chimeric NDV described herein consists of the amino acid sequence set forth in Table 7. In a specific embodiment, a transgene encoding IL-12 in a packaged genome of a chimeric NDV described herein comprises the nucleotide sequence of a sequence set forth in Table 8. In a specific embodiment, a transgene encoding IL-12 in a packaged genome of a chimeric NDV described herein consists of the nucleotide sequence of a sequence set forth in Table 8.

In a specific embodiment, a transgene encoding a derivative of IL-12 in a packaged genome of a chimeric NDV described herein is a derivative of any IL-12 known to those of skill in the art. In a specific embodiment, the IL-12 derivative has at least 70%, 75%, 80%, 85%, 90%, 95%, 96%, 98%, or 99% amino acid sequence identity to an IL-12 known to those of skill in the art. In a specific embodiment, the IL-12 derivative comprises deleted forms of a known IL-12, wherein up to about 20, 15, 10, 9, 8, 7, 6, 5, 4, 3, 2 or 1 amino acid residues are deleted from the known IL-12. Also provided herein are IL-12 derivatives comprising deleted forms of a known IL-12, wherein about 1-3, 3-5, 5-7, 7-10, 10-15, or 15-20 amino acid residues are deleted from the known IL-12. Further provided herein are IL-12 derivatives comprising altered forms of a known IL-12, wherein up to about 20, 15, 10, 9, 8, 7, 6, 5, 4, 3, 2 or 1 amino acid residues of the known IL-12 are substituted (e.g., conservatively substituted) with other amino acids. In some embodiments, the IL-12 derivative comprises up to about 20, 15, 10, 9, 8, 7, 6, 5, 4, 3, 2 or 1 conservatively substituted amino acids (see, e.g., Huang et al., 2016, Preclinical validation: L V/IL-12 transduction of patient leukemia cells for immunotherapy of AML, Molecular Therapy-Methods & Clinical Development, 3, 16074; doi: 10.1038/mtm.2016.74, which is incorporated by reference herein in its entirety). In some embodiments, the conservatively substituted amino acids are not projected to be in the cytokine/receptor interface (see, e.g., Huang et al., 2016, Preclinical validation: LV/IL-12 transduction of patient leukemia cells for immunotherapy of AML, Molecular Therapy Methods & Clinical Development, 3, 16074; doi: 10.1038/mtm.2016.74; Jones & Vignali, 2011, Molecular Interactions within the IL-6/IL-12 cytokine/receptor superfamily, Immunol Res., 51 (1): 5-14, doi: 10.1007/s12026-011-8209-y; each of which is incorporated by reference herein in its entirety). In some embodiments, the IL-12 derivative comprises an IL-12 p35 subunit having the amino acid substitution L165S (i.e., leucine at position 165 of the IL-12 p35 subunit in the IL-12 derivative is substituted with a serine). In some embodiments, the IL-12 derivative comprises an IL-12 p40 subunit having the amino acid substitution of C2G (i.e., cysteine at position 2 of the immature IL-12 p40 subunit (i.e., the IL-12 p40 subunit containing the signal peptide) in the IL-12 derivative is substituted with a glycine).

In a specific embodiment, the IL-12 derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-12. In another specific embodiment, the IL-12 derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-12. In a specific embodiment, the IL-12 derivative comprises an IL-12 p35 subunit that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-12 p35 subunit. In another specific embodiment, the IL-12 derivative is a polypeptide encoded by a nucleic acid sequence, wherein a portion of nucleic acid sequences encodes an IL-12 p35 subunit, wherein said the nucleic acid sequence of said portion is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-12 p35 subunit. In a specific embodiment, the IL-12 derivative comprises an IL-12 p40 subunit that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-12 p40 subunit. In another specific embodiment, the IL-12 derivative is a polypeptide encoded by a nucleic acid sequence, wherein a portion of nucleic acid sequences encodes an IL-12 p40 subunit, wherein said the nucleic acid sequence of said portion is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-12 p40 subunit. In another specific embodiment, the IL-12 derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions and/or substitutions) relative to a native IL-12. In another specific embodiment, the IL-12 derivative is a polypeptide encoded by nucleic acid sequence that can hybridize under high, moderate or typical stringency hybridization conditions to a nucleic acid sequence encoding a native IL-12. In another specific embodiment, the IL-12 derivative is a polypeptide encoded by a nucleic acid sequence that can hybridize under high, moderate or typical stringency hybridization conditions to a nucleic acid sequence encoding a fragment of a native IL-12 of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, the IL-12 derivative is a fragment of a native IL-12. In another specific embodiment, the IL-12 derivative comprises a subunit (e.g., p35 or p40) encoded by a nucleotide sequence that hybridizes over its full length to the nucleotide encoding the native subunit (e.g., native p40 subunit or native p35 subunit). In a specific embodiment, the IL-12 derivative comprises a native IL-12 p40 subunit and a derivative of an IL-12 p35 subunit. In a specific embodiment, the IL-12 derivative comprises a native IL-12 p35 subunit and a derivative of an IL-12 p40 subunit. IL-12 derivatives also include polypeptides that comprise the amino acid sequence of a naturally occurring mature form of IL-12 and a heterologous signal peptide amino acid sequence. In addition, IL-12 derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, IL-12 derivatives include polypeptides comprising one or more non-classical amino acids. In specific embodiments, the IL-12 derivative retains one, two, or more, or all of the functions of the native IL-12 from which it was derived. Tests for determining whether or not an IL-12 derivative retains one or more functions of the native IL-12 from which it was derived are known to one of skill in the art and examples are provided herein.

In specific embodiments, the transgene encoding IL-12 or a derivative thereof in a packaged genome of a chimeric NDV described herein is codon optimized. In a specific embodiment, the nucleotide sequence(s) encoding one or both subunits of a native IL-12 may be codon optimized. A nonlimiting example of a codon-optimized sequence encoding IL-12 p35 or a derivative thereof includes SEQ ID NO:55. Nonlimiting examples of codon-optimized sequences encoding IL-12 p40 or a derivative thereof include SEQ ID NO:54 and 59. Methods of codon optimization are known in the art, e.g., the OptimumGene™ (GenScript®) protocol and U.S. Pat. No. 8,326,547, which is incorporated by reference herein in its entirety.

### 5.3 Construction of NDVS

The NDVs described herein (see, e.g., Sections 5.1, 5.2, and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain) is constructed, inserted into a plasmid vector and engineered to contain a unique restriction site between two transcription units (e.g., the NDV P and M genes; or the NDV HN and L genes). A nucleotide sequence encoding a heterologous amino acid sequence (e.g., an IL-12 transgene or other sequence such as, e.g., a nucleotide sequence encoding an agonist of a co-stimulatory signal and/or an antagonist of an inhibitory signal of an immune cell) may be inserted into the viral genome at the unique restriction site. Alternatively, a nucleotide sequence encoding a heterologous amino acid sequence (e.g., an IL-12 transgene or other sequence such as, e.g., a nucleotide sequence encoding an agonist of a co-stimulatory signal and/or an antagonist of an inhibitory signal of an immune cell) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Techniques for the production of a chimeric NDV that express an antibody are known in the art. See, e.g., Puhler et al., Gene Ther. 15 (5): 371-283 (2008) for the generation of a recombinant NDV expressing a full IgG from two transgenes.

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., García-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning chimeric NDV to encode a transgene and express a heterologous protein encoded by the transgene (e.g., IL-12) are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. Regarding the Rule of Six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a chimeric NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi: 10.3791/50830 (2013) for a discussion of methods for cloning and rescuing of NDV (e.g., chimeric NDV), which is incorporated by reference herein in its entirety.

In a specific embodiment, an NDV described herein (see, e.g., Sections 5.1, 5.2, and 6) can be generated according to a method described in Section 6.

In a specific embodiment, a chimeric NDV comprising a packaged genome comprising a transgene encoding IL-12 or a derivative thereof described herein comprises a La Sota strain backbone. In a specific embodiment, the genomic sequence of the La Sota strain backbone (i.e., without the transgene) is as set forth in SEQ ID NO: 50. In a specific embodiment, a chimeric NDV comprises a packaged genome comprising the nucleotide sequence set forth in SEQ ID Nos: 51, 52 or 60. In a specific embodiment, a chimeric NDV comprises a packaged genome with the nucleotide sequence set forth in SEQ ID Nos: 51, 52 or 60. In some embodiments, a plasmid comprising the nucleotide sequence set forth in a sequence in Table 10 is used to produce a chimeric NDV. In specific embodiments, a plasmid with the nucleotide sequence set forth in a sequence in Table 10 is used to produce a chimeric NDV. See, e.g., Section 6, infra, for techniques that may be used to generate a chimeric NDV using such a plasmid.

### 5.4 Propagation of NDVS

The NDVs described herein (e.g., the chimeric NDVs; see, also, e.g., Sections 5.1, 5.2, and 6) can be propagated in any substrate that allows the virus to grow to titers that permit the uses of the viruses described herein. In one embodiment, the substrate allows the NDVs described herein (e.g., the chimeric NDVs) to grow to titers comparable to those determined for the corresponding wild-type viruses.

The NDVs described herein (e.g., the chimeric NDVs; see, also, e.g., Sections 5.1, 5.2, and 6) may be grown in cells (e.g., avian cells, chicken cells, etc.) that are susceptible to infection by the viruses, embryonated eggs (e.g., chicken eggs or quail eggs) or animals (e.g., birds). Such methods are well-known to those skilled in the art. In a specific embodiment, the NDVs described herein (e.g., the chimeric NDVs) may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the NDVs described herein (e.g., the chimeric NDVs) may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained and/or derived from a human(s). In another embodiment, the NDVs described herein (e.g., the chimeric NDVs) are propagated in chicken cells or embryonated eggs. Representative chicken cells include, but are not limited to, chicken embryo fibroblasts and chicken embryo kidney cells. In a specific embodiment, the NDVs described herein (e.g., the chimeric NDVs) are propagated in Vero cells. In another specific embodiment, the NDVs described herein (e.g., the chimeric NDVs) are propagated in cancer cells in accordance with the methods described in Section 6, infra. In another specific embodiment, the NDVs described herein (e.g., the chimeric NDVs) are propagated in chicken eggs or quail eggs. In certain embodiments, a NDV virus described herein (e.g., a chimeric NDV) is first propagated in embryonated eggs and then propagated in cells (e.g., a cell line).

The NDVs described herein (e.g., the chimeric NDVs) may be propagated in embryonated eggs, e.g., from 6 to 14 days old, 6 to 12 days old, 6 to 10 days old, 6 to 9 days old, 6 to 8 days old, 8 to 10 days old, or 10 to 12 days old. Young or immature embryonated eggs can be used to propagate the NDVs described herein (e.g., the chimeric NDVs). Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. In a specific embodiment, the NDVs described herein (e.g., the chimeric NDVs) are propagated in 10 day old embryonated chicken eggs. The NDVs described herein (e.g., the chimeric NDVs) can be propagated in different locations of the embryonated egg, e.g., the allantoic cavity. For a detailed discussion on the growth and propagation viruses, see, e.g., U.S. Pat. Nos. 6,852,522 and 7,494,808, both of which are hereby incorporated by reference in their entireties.

In a specific embodiment, provided herein is a method for propagating an NDV described herein (e.g., a chimeric NDV described herein), the method comprising culturing a substrate (e.g., a cell line or embryonated egg) infected with the NDV. In another specific embodiment, provided herein is a method for propagating an NDV described herein (e.g., a chimeric NDV described herein), the method comprising: (a) culturing a substrate (e.g., a cell line or embryonated egg) infected with the NDV; and (b) isolating or purifying the NDV from the substrate. In certain embodiments, these methods involve infecting the substrate with the NDV prior to culturing the substrate. See, e.g., Section 6, infra, for methods that may be used to propagate an NDV described herein (e.g., a chimeric NDV described herein).

For virus isolation, the NDVs described herein (e.g., the chimeric NDVs) can be removed from embryonated eggs or cell culture and separated from cellular components, typically by well known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography.

In a specific embodiment, virus isolation from allantoic fluid of an infected egg (e.g., a chicken egg) begins with harvesting allantoic fluid, which is clarified using a filtration system (comprising, e.g., a 1.2 μm glass fiber dead end filtration) to remove cells and other large debris, specifically, comprising a membrane having a net positive charge such that there is a measurable reduction in host cell DNA. The clarified bulk is subsequently processed by tangential flow filtration, e.g., by using 750 kD hollow fiber membranes, concentrating the clarified bulk approximately five-fold. The concentrated clarified bulk is then diafiltered against four diavolumes of high salt buffer, followed by four diavolumes of low salt formulation buffer and subsequently concentrated approximately 10-fold. Accordingly, residual egg proteins, e.g., primarily ovalbumin, and residual DNA are reduced to acceptable levels, and the buffer is exchanged to a buffer compatible with formulation of the chimeric NDV for a composition to be administered to a subject. The resulting product is then sterile filtered through a filter, e.g., a 0.2 μm filter, dispensed into appropriate sterile storage containers, frozen, and stored at −70 degrees Celsius.

In a specific embodiment, an NDV described herein (see, e.g., Sections 5.1, 5.2, and 6) is propagated, isolated, and/or purified according to a method described in Section 6. In a specific embodiment, an NDV described herein (see, e.g., Sections 5.1, 5.2, and 6) is either propagated, isolated, or purified, or any two or all of the foregoing, using a method described in Section 6.

### 5.5 Antagonists of PD-1 or A Ligand Thereof

Also provided herein are antagonists of PD-1 or a ligand thereof for use in combination with an NDV described herein (see, e.g., Section 5.1, Section 5.2, Section 5.5, Section 5.7, and/or Section 6). Any antagonist of PD-1 or a ligand thereof known to one of skill in the art may be utilized in the methods described herein (see, e.g., Section 5.5, Section 5.7 and/or Section 6). Specific examples of ligands for PD-1 include PD-L1 and PD-L2.

In another embodiment, the antagonist of PD-1 or a ligand thereof is an antibody (or an antigen-binding fragment) or a soluble receptor that specifically binds to a ligand of PD-1 (e.g., PD-L1, PD-L2 or both PD-L1 and PD-L2). In a specific embodiment, the antagonist blocks the ligand of PD-1 (e.g., PD-L1, PD-L2 or both PD-L1 and PD-L2) from binding to PD-1 and transducing an inhibitory signal(s). In certain embodiments, the soluble receptor is a fragment of PD-1 or a fragment of a derivative of PD-1 that binds to a ligand of PD-1 (e.g., the extracellular domain of PD-1 or a derivative of PD-1). In some embodiments, the soluble receptor is a fusion protein comprising at least a portion of PD-1 or a derivative of PD-1 (e.g., the extracellular domain of PD-1 or a derivative of PD-1), and a heterologous amino acid sequence. In specific embodiments, the fusion protein comprises at least a portion of PD-1 or a derivative of PD-1, and the Fc portion of an immunoglobulin or a fragment thereof.

In specific embodiments, the antagonist of PD-1 or a ligand thereof is an antibody (or an antigen-binding fragment) that binds to a ligand of PD-1 (e.g., PD-L1, PD-L2 or both PD-L1 and PD-L2). In a specific embodiment, the antagonist blocks (completely or partially) the ligand of PD-1 (e.g., PD-L1, PD-L2 or both PD-L1 and PD-L2) from interacting with PD-1 and transducing an inhibitory signal(s). In certain specific embodiments, the antibody is a monoclonal antibody. In some specific embodiments, the antibody is an scFv. In certain specific embodiments, the antibody is a camelized antibody. In particular embodiments, the antibody is a human or humanized antibody. Nonlimiting examples of antibodies that bind to a ligand of PD-1 include durvalumab (also referred to as “medi-4736”; see, e.g., Lutzky et al., J Clin Oncol. 2014; 32 (suppl 5S): abstr 3001), avelumab (e.g., for Merkel cell carcinoma) (also referred to as “MSB0010718C”; see, e.g., Heery et al. J Clin Oncol. 2014; 32 (suppl 5S): abstr 3064), bms-936559 (see, e.g., Brahmer et al. N. Engl. J. Med. 2012; 366, 2455-2465), and atezolizumab (also referred to as “mpd13280A” or “TECENTRIQ®”; see, e.g., McDermott et al., J Clin Oncol. 2016; 34 (8): 833-842, Herbst et al., J Clin Oncol. 2013; 31 (suppl): abstr 3000, and Full Prescribing Information for TECENTRIQ, Reference ID: 3933242).

In a specific embodiment in which the antagonist of PD-1 or a ligand thereof is an antibody that binds to a ligand of PD-1 (e.g., PD-L1 or PD-L2), the antibody is a PD-1 ligand blocking antibody. In a specific embodiment, the anti-PD-1 ligand antibody (e.g., an anti-PD-L1 or an anti-PD-L2 antibody) specifically binds to the PD-1 ligand (e.g., PD-L1 or PD-L2). In a specific embodiment, the PD-1 ligand is human PD-L1. In another specific embodiment, the PD-1 ligand is human PD-L2. In a specific embodiment, an anti-PD-L1 or anti-PD-L2 antibody inhibits or reduces the interaction between PD-L1 or PD-L2, respectively, and PD-1. In a specific embodiment, an anti-PD-L1 or anti-PD-L2 antibody is an antibody that binds to PD-L1 or PD-L2, respectively, and blocks (completely or partially) the interaction between PD-L1 or PD-L2, respectively, and PD-1, thereby releasing PD-1 pathway-mediated inhibition of the immune response, including the anti-tumor response. In a specific embodiment, the blocking of the interaction between PD-L1 or PD-L2 and PD-1 is complete. In a specific embodiment, the blocking of the interaction between PD-L1 or PD-L2 and PD-1 refers to at least 5%, 10%, 15%, 20%, 25%, 30%, 35%, 40%, 45%, 50%, 55%, 60%, 65%, 70%, 75%, 80%, 85%, 90%, 95%, 98%, or 99% blocking of the interaction between PD-L1 or PD-L2 and PD-1 as assessed by any method known to one of skill in the art, such as, e.g., co-immunoprecipitation or co-localization assays, as compared to interaction between PD-L1 or PD-L2, respectively, and PD-1 in the presence of a negative control therapy (e.g., an anti-IgG antibody). In a specific embodiment, an anti-PD-L1 or anti-PD-L2 antibody inhibits or reduces PD-L1- or PD-L2-dependent, respectively, activation of PD-1. In a specific embodiment, the inhibition of ligand-dependent activation is complete. In a specific embodiment, the inhibition of ligand-dependent activation refers to at least 5%, 10%, 15%, 20%, 25%, 30%, 35%, 40%, 45%, 50%, 55%, 60%, 65%, 70%, 75%, 80%, 85%, 90%, 95%, 98%, or 99% inhibition of ligand-dependent activation as assessed by any method known to one of skill in the art, such as, e.g., a phosphorylation assay, as compared to ligand-dependent PD-1 activation in the presence of a negative control therapy (e.g., an anti-IgG antibody).

In some embodiments, an antagonist of PD-1 or a ligand thereof is an antibody that binds to PD-L1, blocks (completely or partially) the interaction between PD-L1 and PD-1, and blocks (completely or partially) the interaction between PD-L1 and CD80 (B7.1). In certain embodiments, the antibody is an IgG1 monoclonal antibody. In a specific embodiment, the antibody is durvalumab.

In another embodiment, the antagonist of PD-1 or a ligand thereof is an antibody or ligand that binds to PD-1 (e.g., human PD-1), but does not transduce an inhibitory signal(s), as measured by techniques described herein or known to one of skill in the art. In a specific embodiment, the antibody or ligand binds to human PD-1. In a particular embodiment, the antibody or ligand specifically binds to PD-1 (e.g., human PD-1). In certain specific embodiments, the antibody is a monoclonal antibody. In some specific embodiments, the antibody is an scFv. In certain specific embodiments, the antibody is a camelized antibody. In particular embodiments, the antibody is a human or humanized antibody. Nonlimiting examples of antibodies that bind to PD-1 include pembrolizumab (“KEYTRUDA®”; see, e.g., Hamid et al., N Engl J Med. 2013; 369:134-44 and Full Prescribing Information for KEYTRUDA, Reference ID: 3862712), nivolumab (“OPDIVOR”; see, e.g., Topalian et al., N Engl J Med. 2012; 366:2443-54 and Full Prescribing Information for OPDIVO (nivolumab), Reference ID: 3677021), and MEDI0680 (also referred to as “AMP-514”; see, e.g., Hamid et al., Ann Oncol. 2016; 27 (suppl_6): 1050PD). In a preferred embodiment, the anti-PD-1 antibody is pembrolizumab.

In a specific embodiment in which the antagonist of PD-1 or a ligand thereof is an antibody that binds to PD-1 (e.g., human PD-1), the antibody is an anti-PD-1 antibody that blocks or reduces the interaction between PD-1 (e.g., human PD-1) and a ligand thereof (e.g., PD-L1, PD-L2 or both PD-L1 and PD-1.2). In a specific embodiment, a PD-1 blocking antibody binds to PD-1 and inhibits or reduces the interaction between PD-1 (e.g., human PD-1) and a ligand thereof (e.g., either PD-L1, PD-L2, or both). In a specific embodiment, the PD-1 blocking antibody specifically binds to PD-1. In some embodiments, an anti-PD-1 is an antibody that binds to PD-1 (e.g., human PD-1) and blocks (completely or partially) the interaction between PD-1 (e.g., human PD-1) and either PD-L1, PD-L2, or both, thereby releasing PD-1 pathway-mediated inhibition of the immune response, including the anti-tumor response. In a specific embodiment, an anti-PD-1 is an antibody that binds to PD-1 (e.g., human PD-1) and blocks (completely or partially) the interaction between PD-1 (e.g., human PD-1) and its ligands, PD-L1 and PD-L2, thereby releasing PD-1 pathway-mediated inhibition of the immune response, including the anti-tumor response. In a specific embodiment, the blocking of the interaction between PD-1 (e.g., human PD-1) and either PD-L1, PD-L2, or both is complete. In a specific embodiment, the blocking of the interaction between PD-1 (e.g., human PD-1) and either PD-L1, PD-L2, or both refers to at least 5%, 10%, 15%, 20%, 25%, 30%, 35%, 40%, 45%, 50%, 55%, 60%, 65%, 70%, 75%, 80%, 85%, 90%, 95%, 98%, or 99% blocking of the interaction between PD-1 (e.g., human PD-1) and either PD-L1, PD-L2, or both as assessed by any method known to one of skill in the art, such as, e.g., co-immunoprecipitation or co-localization assays, as compared to interaction between PD-1 (e.g., human PD-1) and either PD-L1, PD-L2, or both in the presence of a negative control therapy (e.g., an anti-IgG antibody). In a specific embodiment, an anti-PD-1 antibody inhibits ligand-dependent (e.g., either PD-L1, PD-L2, or both) activation of PD-1 (e.g., human PD-1). In a specific embodiment, the inhibition of ligand-dependent activation is complete. In a specific embodiment, the inhibition of ligand-dependent activation refers to at least 5%, 10%, 15%, 20%, 25%, 30%, 35%, 40%, 45%, 50%, 55%, 60%, 65%, 70%, 75%, 80%, 85%, 90%, 95%, 98%, or 99% inhibition of ligand-dependent activation as assessed by any method known to one of skill in the art, such as, e.g., a phosphorylation assay, as compared to ligand-dependent PD-1 (e.g., human PD-1) activation in the presence of a negative control therapy (e.g., an anti-IgG antibody).

In another embodiment, the antagonist of PD-1 or a ligand thereof is a ligand that binds to PD-1 (e.g., human PD-1), but does not transduce an inhibitory signal(s). In another embodiment, the antagonist of PD-1 or a ligand thereof is a ligand that binds to PD-1 (e.g., human PD-1), but only nominally transduces an inhibitory signal(s). In certain specific embodiments, the ligand is a fusion protein comprising at least a portion of a ligand of PD-1 (e.g., human PD-1) or a derivative of a ligand of PD-1, and a heterologous amino acid sequence. In specific embodiments, the fusion protein comprises at least a portion of a ligand of PD-1 or a derivative of a ligand of PD-1, and the Fc portion of an immunoglobulin or a fragment thereof. An example of such a fusion protein is AMP-224 (see, e.g., Infante et al., J Clin Oncol. 2013; 31 (suppl): abstr 3044).

In some embodiments, an antagonist of PD-1 or a ligand thereof (e.g., selectively) inhibits or reduces one or more of the signal transduction pathways induced by the binding of PD-1 (e.g., human PD-1) to its ligand(s). In specific embodiments, an antagonist of PD-1 or a ligand thereof inhibits or reduces one or more of the signal transduction pathways induced by the binding of PD-1 (e.g., human PD-1) to one or more of its ligands (e.g., either PD-L1, PD-L2, or both) by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of PD-1 (e.g., human PD-1) to one or more of its ligands (e.g., either PD-L1, PD-1.2, or both) in the absence of the antagonist. In specific embodiments, an antagonist of PD-1 or its ligand: (i) inhibits or reduces one or more of the signal transduction pathways induced by the binding of PD-1 (e.g., human PD-1) to one particular ligand (e.g., PD-L1) by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of PD-1 (e.g., human PD-1) to the one particular ligand (e.g., PDL1) in the absence of the antagonist; and (ii) does not inhibit or reduce, or inhibits or reduces one or more of the signal transduction pathways induced by the binding of PD-1 (e.g., human PD-1) to one or more other ligands (e.g., PD-L2) by less than 20%, 15%, 10%, 5%, or 2%, or in the range of between 2% to 5%, 2% to 10%, 5% to 10%, 5% to 15%, 5% to 20%, 10% to 15%, or 15% to 20% relative to the one or more signal transduction pathways induced by the binding of PD-1 to such one or more other ligands in the absence of the antagonist.

In specific embodiments, an antagonist of PD-1 or a ligand thereof inhibits or reduces one or more of the signal transduction pathways induced by the binding of PD-1 (e.g., human PD-1) to either PD-L1, PD-L2, or both by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of PD-1 (e.g., human PD-1) to either PD-L1, PD-L2, or both in the absence of the antagonist. In specific embodiments, an antagonist of PD-1 or a ligand thereof induces, activates and/or enhances one or more immune activities, functions or responses. The one or more immune activities, functions or responses can be in the form of, e.g., an antibody response (humoral response) or a cellular immune response, e.g., cytokine secretion (e.g., interferon-gamma), helper activity or cellular cytotoxicity. In one embodiment, expression of an activation marker on immune cells (e.g., CD44, Granzyme, or Ki-67), expression of a co-stimulatory receptor on immune cells (e.g., ICOS, CD28, OX40, or CD27), expression of a ligand for a co-stimulatory receptor (e.g., B7HRP1, CD80, CD86, OX40L, or CD70), cytokine secretion, infiltration of immune cells (e.g., T-lymphocytes, B lymphocytes and/or NK cells) to a tumor, antibody production, effector function, T cell activation, T cell differentiation, T cell proliferation, B cell differentiation, B cell proliferation, and/or NK cell proliferation is induced, activated and/or enhanced following contact with an antagonist of PD-1 or a ligand thereof. In another embodiment, myeloid-derived suppressor cell (MDSC) tumor infiltration and proliferation, Treg tumor infiltration, activation and proliferation, peripheral blood MDSC and Treg counts are inhibited following contact with an antagonist of PD-1 or a ligand thereof. In another embodiment, expression of a ligand of PD-1 (e.g., PD-L1, PD-1.2 or both PD-L1 and PD-L2) is induced, activated and/or enhanced following contact with an antagonist of PD-1 or a ligand thereof. In a particular embodiment, the expression of PD-L1 is induced and/or increased following contact with an antagonist of PD-1 or a ligand thereof. In another embodiment, one, two, or more of the effects described in Section 6, infra, occurs following contact with an antagonist of PD-1 or a ligand thereof.

Nonlimiting examples of antagonists of PD-1 or a ligand thereof include pembrolizumab (“KEYTRUDA®”; see, e.g., Hamid et al., N Engl J Med. 2013; 369:134-44 and Full Prescribing Information for KEYTRUDA (pembrolizumab), Reference ID: 3862712), nivolumab (“OPDIVO®”; see, e.g., Topalian et al., N Engl J Med. 2012; 366:2443-54 and Full Prescribing Information for OPDIVO (nivolumab), Reference ID: 3677021), AMP-224 (see, e.g., Infante et al., J Clin Oncol. 2013; 31 (suppl): abstr 3044), MEDI0680 (also referred to as “AMP-514”; see, e.g., Hamid et al., Ann Oncol. 2016; 27 (suppl_6): 1050PD), durvalumab (also referred to as “medi-4736”; see, e.g., Lutzky et al., J Clin Oncol. 2014; 32 (suppl 5S): abstr 3001), avelumab (e.g., for Merkel cell carcinoma) (also referred to as “MSB0010718C”; see, e.g., Heery et al. J Clin Oncol. 2014; 32 (suppl 5S): abstr 3064), bms-936559 (see, e.g., Brahmer et al. N. Engl. J. Med. 2012; 366, 2455-2465), and atezolizumab (also referred to as “mpdl3280A” and “TECENTRIQ®”; see, e.g., McDermott et al., J Clin Oncol. 2016; 34 (8): 833-842, Herbst et al., J Clin Oncol. 2013; 31 (suppl): abstr 3000, and Full Prescribing Information for TECENTRIQ, Reference ID: 3933242). In a specific embodiment, the antagonist of PD-1 or a ligand thereof is a therapy approved by the U.S. FDA for treatment of one or more cancers. Nonlimiting examples of an antagonist of PD-1 or a ligand thereof approved by the U.S. FDA for treatment of cancer include pembrolizumab, nivolumab, atezolizumab, and avelumab. In a specific embodiment, the antagonist of PD-1 or a ligand thereof is a therapy approved by the EMA for treatment of one or more cancers. Nonlimiting examples of an antagonist of PD-1 or a ligand thereof approved by the EMA for treatment of cancer include pembrolizumab, nivolumab, and atezolizumab. In a specific embodiment, the antagonist of PD-1 or a ligand thereof is nivolumab. In a specific embodiment, the antagonist of PD-1 or a ligand thereof is anti-PD-1 antibody described in International Patent Application Publication No WO 2008/156712, or U.S. U.S. Pat. Nos. 8,354,509, 8,952,136, or U.S. Pat. No. 8,900,587, each of which is incorporated by reference in its entirety. In a preferred embodiment, the antagonist of PD-1 or a ligand thereof is pembrolizumab.

In a specific embodiment, provided herein is an antibody that binds to human PD-1, which antibody comprises a variable light chain region (VLCR) complementarity determining region (CDR) 1 comprising the amino acid sequence RASKGVSTSGYSYLH (SEQ ID NO: 1), a VLCR CDR2 comprising the amino acid sequence LASYLES (SEQ ID NO: 2), a VLCR CDR3 comprising the amino acid sequence QHSRDLPLT (SEQ ID NO: 3), a variable heavy chain region (VHCR) CDR1 comprising the amino acid sequence NYYMY (SEQ ID NO: 6), a VHCR CDR2 comprising the amino acid sequence GINPSNGGTNFNEKFKN (SEQ ID NO: 7), and a VHCR CDR3 comprising the amino acid sequence RDYRFDMGFDY (SEQ ID NO: 8). In another specific embodiment, provided herein is an antibody that binds to human PD-1, which antibody comprises (a) a VLCR comprising (i) a VLCR CDR1 comprising the amino acid sequence RASKGVSTSGYSYLH (SEQ ID NO: 1), (ii) a VLCR CDR2 comprising the amino acid sequence LASYLES (SEQ ID NO: 2), and (iii) a VLCR CDR3 comprising the amino acid sequence QHSRDLPLT (SEQ ID NO: 3); and (b) a VHCR comprising (i) a VHCR CDR1 comprising the amino acid sequence NYYMY (SEQ ID NO: 6), (ii) a VHCR CDR2 comprising the amino acid sequence GINPSNGGTNFNEKFKN (SEQ ID NO: 7), and (iii) a VHCR CDR3 comprising the amino acid sequence RDYRFDMGFDY (SEQ ID NO: 8). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1, which antibody comprises: (a) a VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a VHCR comprising (i) a VHCR CDR1 comprising the amino acid sequence NYYMY (SEQ ID NO: 6), (ii) a VHCR CDR2 comprising the amino acid sequence GINPSNGGTNFNEKFKN (SEQ ID NO: 7), and (iii) a VHICR CDR3 comprising the amino acid sequence RDYRFDMGFDY (SEQ ID NO: 8). In another specific embodiment, provided herein is an antibody that binds to human PD-1, which antibody comprises: (a) a VLCR comprising (i) a VLCR CDR1 comprising the amino acid sequence RASKGVSTSGYSYLH (SEQ ID NO: 1), (ii) a VLCR CDR2 comprising the amino acid sequence LASYLES (SEQ ID NO: 2), and (iii) a VLCR CDR3 comprising the amino acid sequence QHSRDLPLT (SEQ ID NO: 3); and (b) a VHCR comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSS (SEQ ID NO: 9). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a preferred embodiment, provided herein is an antibody that binds to human PD-1, which antibody comprises: (a) a VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a VHCR comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSS (SEQ ID NO: 9). In a preferred embodiment, the antibody is an IgG4 kappa immunoglobulin.

In a specific embodiment, provided herein is an antibody that binds to human PD-1 and blocks the interaction between human PD-1 and its ligands, PD-L1 and PD-L2, which antibody comprises a variable light chain region (VLCR) complementarity determining region (CDR) 1 comprising the amino acid sequence RASKGVSTSGYSYLH (SEQ ID NO: 1), a VLCR CDR2 comprising the amino acid sequence LASYLES (SEQ ID NO: 2), a VLCR CDR3 comprising the amino acid sequence QHSRDLPLT (SEQ ID NO: 3), a variable heavy chain region (VHCR) CDR1 comprising the amino acid sequence NYYMY (SEQ ID NO: 6), a VHCR CDR2 comprising the amino acid sequence GINPSNGGTNFNEKFKN (SEQ ID NO: 7), and a VHCR CDR3 comprising the amino acid sequence RDYRFDMGEDY (SEQ ID NO: 8). In another specific embodiment, provided herein is an antibody that binds to human PD-1 and blocks the interaction between human PD-1 and its ligands, PD-L1 and PD-L2, which antibody comprises (a) a VLCR comprising (i) a VLCR CDR1 comprising the amino acid sequence RASKGVSTSGYSYLH (SEQ ID NO: 1), (ii) a VLCR CDR2 comprising the amino acid sequence LASYLES (SEQ ID NO: 2), and (iii) a VLCR CDR3 comprising the amino acid sequence QHSRDLPLT (SEQ ID NO: 3); and (b) a VHCR comprising (i) a VHCR CDR1 comprising the amino acid sequence NYYMY (SEQ ID NO: 6), (ii) a VHCR CDR2 comprising the amino acid sequence GINPSNGGTNFNEKFKN (SEQ ID NO: 7), and (iii) a VHCR CDR3 comprising the amino acid sequence RDYRFDMGFDY (SEQ ID NO: 8). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1 and blocks the interaction between human PD-1 and its ligands, PD-L1 and PD-L2, which antibody comprises: (a) a VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a VHCR comprising (i) a VHCR CDR1 comprising the amino acid sequence NYYMY (SEQ ID NO: 6), (ii) a VHCR CDR2 comprising the amino acid sequence GINPSNGGTNFNEKFKN (SEQ ID NO: 7), and (iii) a VHCR CDR3 comprising the amino acid sequence RDYRFDMGFDY (SEQ ID NO: 8). In another specific embodiment, provided herein is an antibody that binds to human PD-1 and blocks the interaction between human PD-1 and its ligands, PD-L1 and PD-L2, which antibody comprises: (a) a VLCR comprising (i) a VLCR CDR1 comprising the amino acid sequence RASKGVSTSGYSYLH (SEQ ID NO: 1), (ii) a VLCR CDR2 comprising the amino acid sequence LASYLES (SEQ ID NO: 2), and (iii) a VLCR CDR3 comprising the amino acid sequence QHSRDLPLT (SEQ ID NO: 3); and (b) a VHCR comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSS (SEQ ID NO: 9). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1 and blocks the interaction between human PD-1 and its ligands, PD-L1 and PD-L2, which antibody comprises: (a) a VLCR CDR1, a VLCR CDR2, and a VLCR CDR3 comprising the amino acid sequences of the VLCR CDR1, VLCR CDR2, and VLCR CDR3, respectively, of the VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a VHCR CDR1, a VHCR CDR2, and a VHCR CDR3 comprising the amino acid sequences of the VHCR CDR1, VHCR CDR2, and VHCR CDR3, respectively, of the VHCR comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGEDYWGQG TTVTVSS (SEQ ID NO: 9). In certain aspects, the CDRs of an antibody can be determined according to the Kabat numbering system. In a specific embodiment, the VLCR CDR1 comprising the amino acid sequence RASKGVSTSGYSYLH (SEQ ID NO: 1), the VLCR CDR2 comprising the amino acid sequence LASYLES (SEQ ID NO: 2), the VLCR CDR3 comprising the amino acid sequence QHSRDLPLT (SEQ ID NO: 3), the VHCR CDR1 comprising the amino acid sequence NYYMY (SEQ ID NO: 6), the VHCR CDR2 comprising the amino acid sequence GINPSNGGTNFNEKFKN (SEQ ID NO: 7), and the VHCR CDR3 comprising the amino acid sequence RDYRFDMGFDY (SEQ ID NO: 8), as determined according to the Kabat numbering system. In some aspects, the CDRs of an antibody can be determined according to the Chothia numbering scheme, which refers to the location of immunoglobulin structural loops (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226). In certain aspects, the CDRs of an antibody can be determined according to the IMGT numbering system as described in Lefranc, M.-P., 1999, The Immunologist, 7:132-136 and Lefranc, M.-P. et al., 1999, Nucleic Acids Res., 27:209-212. In certain aspects, the CDRs of an antibody can be determined according to MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001). In certain aspects, the CDRs of an antibody can be determined according to the AbM numbering scheme, which refers AbM hypervariable regions, which represent a compromise between the Kabat CDRs and Chothia structural loops, and are used by Oxford Molecular's AbM antibody modeling software. In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

The terms “Kabat numbering,” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). With respect to the Kabat numbering system, (i) the VH CDR1 is typically present at amino acid positions 31 to 35 of the heavy chain, which can optionally include one or two additional amino acids following amino acid position 35 (referred to in the Kabat numbering scheme as 35A and 35B); (ii) the VH CDR2 is typically present at amino acid positions 50 to 65 of the heavy chain; and (iii) the VH CDR2 is typically present at amino acid positions 95 to 102 of the heavy chain (Kabat, Elvin A. et al., Sequences of Proteins of Immunological Interest. Bethesda: National Institutes of Health, 1983). With respect to the Kabat numbering system, (i) the VL CDR1 is typically present at amino acid positions 24 to 34 of the light chain; (ii) the VL CDR2 is typically present at amino acid positions 50 to 56 of the light chain; and (iii) the VL CDR3 is typically present at amino acid positions 89 to 97 of the light chain (Kabat, Elvin A. et al., Sequences of Proteins of Immunological Interest. Bethesda: National Institutes of Health, 1983). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The Chothia definition is based on the location of the structural loop regions (Chothia et al., (1987) J Mol Biol 196:901-917; and U.S. Pat. No. 7,709,226). The term “Chothia CDRs,” and like terms are recognized in the art and refer to antibody CDR sequences as determined according to the method of Chothia and Lesk, 1987, J. Mol. Biol., 196:901-917, which will be referred to herein as the “Chothia CDRs” (see also, e.g., U.S. Pat. No. 7,709,226 and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)). With respect to the Chothia numbering system, using the Kabat numbering system of numbering amino acid residues in the VH region, (i) the VH CDR1 is typically present at amino acid positions 26 to 32 of the heavy chain; (ii) the VH CDR2 is typically present at amino acid positions 53 to 55 of the heavy chain; and (iii) the VH CDR3 is typically present at amino acid positions 96 to 101 of the heavy chain. In a specific embodiment, with respect to the Chothia numbering system, using the Kabat numbering system of numbering amino acid residues in the VH region, (i) the VH CDR1 is typically present at amino acid positions 26 to 32 or 34 of the heavy chain; (ii) the VH CDR2 is typically present at amino acid positions 52 to 56 (in one embodiment, CDR2 is at positions 52A-56, wherein 52A follows position 52) of the heavy chain; and (iii) the VH CDR3 is typically present at amino acid positions 95 to 102 of the heavy chain (in one embodiment, there is no amino acid at positions numbered 96-100). With respect to the Chothia numbering system, using the Kabat numbering system of numbering amino acid residues in the VL region, (i) the VL CDR1 is typically present at amino acid positions 26 to 33 of the light chain; (ii) the VL CDR2 is typically present at amino acid positions 50 to 52 of the light chain; and (iii) the VL CDR3 is typically present at amino acid positions 91 to 96 of the light chain. In a specific embodiment, with respect to the Chothia numbering system, using the Kabat numbering system of numbering amino acid residues in the VL region, (i) the VL CDR1 is typically present at amino acid positions 24 to 34 of the light chain; (ii) the VL CDR2 is typically present at amino acid positions 50 to 56 of the light chain; and (iii) the VL CDR3 is typically present at amino acid positions 89 to 97 of the light chain (in one embodiment, there is no amino acid at positions numbered 96-100). These Chothia CDR positions may vary depending on the antibody, and may be determined according to methods known in the art.

The IMGT definition is from the IMGT (“IMGT®, the international ImMunoGene Tics information System® website imgt.org, founder and director: Marie-Paule Lefranc, Montpellier, France; see, e.g., Lefranc, M.-P., 1999, The Immunologist, 7:132-136 and Lefranc, M.-P. et al., 1999, Nucleic Acids Res., 27:209-212, both of which are incorporated herein by reference in their entirety). With respect to the IMGT numbering system, (i) the VH CDR1 is typically present at amino acid positions 25 to 35 of the heavy chain; (ii) the VH CDR2 is typically present at amino acid positions 51 to 57 of the heavy chain; and (iii) the VH CDR2 is typically present at amino acid positions 93 to 102 of the heavy chain. With respect to the IMGT numbering system, (i) the VL CDR1 is typically present at amino acid positions 27 to 32 of the light chain; (ii) the VL CDR2 is typically present at amino acid positions 50 to 52 of the light chain; and (iii) the VL CDR3 is typically present at amino acid positions 89 to 97 of the light chain.

In a preferred embodiment, provided herein is an antibody that binds to human PD-1 and blocks the interaction between human PD-1 and its ligands, PD-L1 and PD-L2, which antibody comprises: (a) a VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a VHCR comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSS (SEQ ID NO: 9). In a preferred embodiment, the antibody is an IgG4 kappa immunoglobulin.

In a specific embodiment, provided herein is an antibody that binds to human PD-1 and blocks the interaction between human PD-1 and its ligands, PD-L1 and PD-L2, which antibody comprises: (a) a light chain comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIKRTVAAPSVFI FPPSDEQLKSGTASVVCLLNNFYPREAKVQWKVDNALQSGNSQESVTEQDSKDSTYSLS STLTLSKADYEKHKVYACEVTHQGLSSPVTKSENRGEC (SEQ ID NO: 5); and (b) a heavy chain comprising a VHCR comprising the amino acid sequence QVQLVQSGVEVKKPGASVKVSCKASGYTFTNYYMYWVRQAPGQGLEWMGGINPSNG GTNFNEKFKNRVTITTDSSTTTAYMELKSLQFDDTAVYYCARRDYRFDMGFDYWGQG TTVTVSS (SEQ ID NO: 9). In another specific embodiment, provided herein is an antibody that binds to human PD-1 and blocks the interaction between human PD-1 and its ligands, PD-L1 and PD-L2, which antibody comprises: (a) a light chain comprising a VLCR comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIK (SEQ ID NO: 4); and (b) a heavy chain comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPCSRSTSESTAALGCLVKDYFPEPVTVSWNSGALTSGVHTF PAVLQSSGLYSLSSVVTVPSSSLGTKTYTCNVDHKPSNTKVDKRVESKYGPPCPPCPAPE FLGGPSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLT VDKSRWQEGNVFSCSVMHEALHNHYTQKSLSLSLGK (SEQ ID NO: 10).

In a preferred embodiment, provided herein is an antibody that binds to human PD-1 and blocks the interaction between human PD-1 and its ligands, PD-L1 and PD-L2, which antibody comprises: (a) a light chain comprising the amino acid sequence EIVLTQSPATLSLSPGERATLSCRASKGVSTSGYSYLHWYQQKPGQAPRLLIYLASYLES GVPARFSGSGSGTDFTLTISSLEPEDFAVYYCQHSRDLPLTFGGGTKVEIKRTVAAPSVFI FPPSDEQLKSGTASVVCLLNNFYPREAKVQWKVDNALQSGNSQESVTEQDSKDSTYSLS STLTLSKADYEKHKVYACEVTHQGLSSPVTKSFNRGEC (SEQ ID NO: 5); and (b) a heavy chain comprising the amino acid sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPCSRSTSESTAALGCLVKDYFPEPVTVSWNSGALTSGVHTF PAVLQSSGLYSLSSVVTVPSSSLGTKTYTCNVDHKPSNTKVDKRVESKYGPPCPPCPAPE FLGGPSVFLFPPKPKDTLMISRTPEVTCVVVDVSQEDPEVQFNWYVDGVEVHNAKTKPR EEQFNSTYRVVSVLTVLHIQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLT VDKSRWQEGNVFSCSVMHEALHNHYTQKSLSLSLGK (SEQ ID NO: 10).

In a specific embodiment, provided herein is an antibody that binds to human PD-1, which antibody comprises a VLCR CDR1 comprising the amino acid sequence RASQSVSSYLA (SEQ ID NO: 11), a VLCR CDR2 comprising the amino acid sequence DASNRAT (SEQ ID NO: 12), a VLCR CDR3 comprising the amino acid sequence QQSSNWPRT (SEQ ID NO: 13), a VHCR CDR1 comprising the amino acid sequence NSGMH (SEQ ID NO: 16), a VHCR CDR2 comprising the amino acid sequence VIWYDGSKRYYADSVKG (SEQ ID NO: 17), and a VHCR CDR3 comprising the amino acid sequence NDDY (SEQ ID NO: 18). In another specific embodiment, provided herein is an antibody that binds to human PD-1, which antibody comprises: (a) a VLCR comprising (i) a VLCR CDR1 comprising the amino acid sequence RASQSVSSYLA (SEQ ID NO: 11), (ii) a VLCR CDR2 comprising the amino acid sequence DASNRAT (SEQ ID NO: 12), and (iii) a VLCR CDR3 comprising the amino acid sequence QQSSNWPRT (SEQ ID NO: 13); and (b) a VHICR comprising (i) a VHICR CDR1 comprising the amino acid sequence NSGMH (SEQ ID NO: 16), (ii) a VHCR CDR2 comprising the amino acid sequence VIWYDGSKRYYADSVKG (SEQ ID NO: 17), and (iii) a VHCR CDR3 comprising the amino acid sequence NDDY (SEQ ID NO: 18). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1, which antibody comprises: (a) a VLCR comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIK (SEQ ID NO: 14); and (b) a VHCR comprising (i) a VHCR CDR1 comprising the amino acid sequence NSGMH (SEQ ID NO: 16), (ii) a VHCR CDR2 comprising the amino acid sequence VIWYDGSKRYYADSVKG (SEQ ID NO: 17), and (iii) a VHCR CDR3 comprising the amino acid sequence NDDY (SEQ ID NO: 18). In another specific embodiment, provided herein is an antibody that binds to human PD-1, which antibody comprises: (a) a VLCR comprising (i) a VLCR CDR1 comprising the amino acid sequence RASQSVSSYLA (SEQ ID NO: 11), (ii) a VLCR CDR2 comprising the amino acid sequence DASNRAT (SEQ ID NO: 12), and (iii) a VLCR CDR3 comprising the amino acid sequence QQSSNWPRT (SEQ ID NO: 13); and (b) a VHCR comprising QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSS (SEQ ID NO: 19). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1 and blocks the binding of human PD-1 to its ligands, PD-L1 and PD-L2, which antibody comprises a VLCR CDR1 comprising the amino acid sequence RASQSVSSYLA (SEQ ID NO: 11), a VLCR CDR2 comprising the amino acid sequence DASNRAT (SEQ ID NO: 12), a VLCR CDR3 comprising the amino acid sequence QQSSNWPRT (SEQ ID NO: 13), a VHCR CDR1 comprising the amino acid sequence NSGMH (SEQ ID NO: 16), a VHCR CDR2 comprising the amino acid sequence VIWYDGSKRYYADSVKG (SEQ ID NO: 17), and a VHCR CDR3 comprising the amino acid sequence NDDY (SEQ ID NO: 18). In another specific embodiment, provided herein is an antibody that binds to human PD-1 and blocks the binding of human PD-1 to its ligands, PD-L1 and PD-L2, which antibody comprises: (a) a VLCR comprising (i) a VLCR CDR1 comprising the amino acid sequence RASQSVSSYLA (SEQ ID NO: 11), (ii) a VLCR CDR2 comprising the amino acid sequence DASNRAT (SEQ ID NO: 12), and (iii) a VLCR CDR3 comprising the amino acid sequence QQSSNWPRT (SEQ ID NO: 13); and (b) a VHCR comprising (i) a VHCR CDR1 comprising the amino acid sequence NSGMH (SEQ ID NO: 16), (ii) a VHCR CDR2 comprising the amino acid sequence VIWYDGSKRYYADSVKG (SEQ ID NO: 17), and (iii) a VHCR CDR3 comprising the amino acid sequence NDDY (SEQ ID NO: 18). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1 and blocks the binding of human PD-1 to its ligands, PD-L1 and PD-L2, which antibody comprises:
(a) a VLCR comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIK (SEQ ID NO: 14);
and (b) a VHCR comprising (i) a VHCR CDR1 comprising the amino acid sequence NSGMH (SEQ ID NO: 16), (ii) a VHCR CDR2 comprising the amino acid sequence VIWYDGSKRYYADSVKG (SEQ ID NO: 17), and (iii) a VHCR CDR3 comprising the amino acid sequence NDDY (SEQ ID NO: 18). In another specific embodiment, provided herein is an antibody that binds to human PD-1 and blocks the binding of human PD-1 to its ligands, PD-L1 and PD-L2, which antibody comprises: (a) a VLCR comprising (i) a VLCR CDR1 comprising the amino acid sequence RASQSVSSYLA (SEQ ID NO: 11), (ii) a VLCR CDR2 comprising the amino acid sequence DASNRAT (SEQ ID NO: 12), and (iii) a VLCR CDR3 comprising the amino acid sequence QQSSNWPRT (SEQ ID NO: 13); and (b) a VHICR comprising QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSS (SEQ ID NO: 19). In a specific embodiment, the antibody is an IgG (e.g., an IgG1, IgG2, IgG3, or IgG4) antibody. In a preferred embodiment, the antibody is an IgG4 antibody. In certain embodiments, the antibody is a humanized or chimeric antibody. In a preferred embodiment, the antibody is a humanized monoclonal antibody. In another preferred embodiment, the antibody is an IgG4 kappa immunoglobulin.

In a specific embodiment, provided herein is an antibody that binds to human PD-1 and blocks the binding of human PD-1 to its ligands, PD-L1 and PD-L2, which antibody comprises:
(a) a VLCR CDR1, a VLCR CDR2, and a VLCR CDR3 comprising the amino acid sequences of the VLCR CDR1, VLCR CDR2, and VLCR CDR3, respectively, of the VLCR comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIK (SEQ ID NO: 14);
and (b) a VHCR CDR1, a VHCR CDR2, and a VHCR CDR3 comprising the amino acid sequences of the VHCR CDR1, VHICR CDR2, and VHICR CDR3, respectively, of the VHCR comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSS (SEQ ID NO: 19). In certain aspects, the CDRs of an antibody can be determined according to the Kabat numbering system. In some aspects, the CDRs of an antibody can be determined according to the Chothia numbering scheme. In certain aspects, the CDRs of an antibody can be determined according to MacCallum et al., 1996, J. Mol. Biol., 262:732-745. In some aspects, the CDRs of an antibody can be determined according to the IMGT numbering system as described in Lefranc, M.-P., 1999, The Immunologist, 7:132-136 and Lefranc, M.-P. et al., 1999, Nucleic Acids Res., 27:209-212. In certain aspects, the CDRs of an antibody can be determined according to the AbM numbering scheme. In a specific embodiment, the antibody is an IgG (e.g., an IgG1, IgG2, IgG3, or IgG4) antibody. In a preferred embodiment, the antibody is an IgG4 antibody. In another preferred embodiment, the antibody is an IgG4 kappa immunoglobulin.

In a specific embodiment, provided herein is an antibody that binds to human PD-1 and blocks the binding of human PD-1 to its ligands, PD-L1 and PD-1.2, which antibody comprises:
(a) a VLCR comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIK (SEQ ID NO: 14);
and (b) a VHCR comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSS (SEQ ID NO: 19). In a specific embodiment, the antibody is an IgG (e.g., an IgG1, IgG2, IgG3, or IgG4) antibody. In a preferred embodiment, the antibody is an IgG4 antibody. In another preferred embodiment, the antibody is an IgG4 kappa immunoglobulin.

In a specific embodiment, provided herein is an antibody that binds to human PD-1 and blocks the interaction between human PD-1 and its ligands, PD-L1 and PD-L2, which antibody comprises: (a) a light chain comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIKRTVAAPSVFIFPPS DEQLKSGTASVVCLLNNFYPREAKVQWKVDNALQSGNSQESVTEQDSKDSTYSLSSTL TLSKADYEKHKVYACEVTHQGLSSPVTKSENRGEC (SEQ ID NO: 15); and (b) a heavy chain comprising a VHCR comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSS (SEQ ID NO: 19). In another specific embodiment, provided herein is an antibody that binds to human PD-1 and and blocks the interaction between human PD-1 and its ligands, PD-L1 and PD-L2, which antibody comprises: (a) a light chain comprising a VLCR comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIK (SEQ ID NO: 14) and
(b) a heavy chain comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTL VTVSSAS TKGPSVFPLAPCSRSTSESTAALGCLVKDYFPEPVTVSWNSGALTSGVHTFPAVLQSSGL YSLSSVVTVPSSSLGTKTYTCNVDHKPSNTKVDKRVESKYGPPCPPCPAPEFLGGPSVFL FPPKPKDTILMISRTPEVTCVVVDVSQEDPEVQFNWYVDGVEVHNAKTKPREEQFNSTY RVVSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFFLYSRLTVDKSRWQ EGNVFSCSVMHEALHNHYTQKSLSLSLGK (SEQ ID NO: 20).

In a preferred embodiment, provided herein is an antibody that binds to human PD-1 and and blocks the interaction between human PD-1 and its ligands, PD-L1 and PD-L2, which antibody comprises: (a) a light chain comprising the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIKRTVAAPSVFIFPPS DEQLKSGTASVVCLLNNFYPREAKVQWKVDNALQSGNSQESVTEQDSKDSTYSLSSTL TLSKADYEKHKVYACEVTHQGLSSPVTKSFNRGEC (SEQ ID NO: 15) and (b) a heavy chain comprising the amino acid sequence QVQLVESGGGVVQPGRSLRLDCKASGITFSNSGMHWVRQAPGKGLEWVAVIWYDGSK RYYADSVKGRFTISRDNSKNTLFLQMNSLRAEDTAVYYCATNDDYWGQGTLVTVSSAS TKGPSVFPLAPCSRSTSESTAALGCLVKDYFPEPVTVSWNSGALTSGVHTFPAVLQSSGL YSLSSVVTVPSSSLGTKTYTCNVDHKPSNTKVDKRVESKYGPPCPPCPAPEFLGGPSVFL FPPKPKDTLMISRTPEVTCVVVDVSQEDPEVQFNWYVDGVEVINAKTKPREEQFNSTY RVVSVLTVLHQDWLNGKEYKCKVSNKGLPSSIEKTISKAKGQPREPQVYTIPPSQEEMT KNQVSLTCLVKGFYPSDIAVEWESNGQPENNYKTTPPVLDSDGSFFLYSRLTVDKSRWQ EGNVFSCSVMHEALHNHYTQKSLSLSLGK (SEQ ID NO: 20).

Antagonists of PD-1 or a ligand thereof may be produced by techniques known by one skilled in the art. See, e.g., U.S. Pat. Nos. 9,642,298, 8,952,136, 8,900,587, 8,168,757, 7,488,802, U.S. Patent Application Publication No. 2016/0137721, and International Patent Application Publication No. WO 2014/159960 for examples of techniques that may be used to produce antagonists of PD-1; or a ligand thereof. In a specific embodiment, an antagonist of PD-1 or a ligand thereof is isolated.

### 5.5.1 Antibody Production

In one aspect, provided herein are methods for making an antibody or other proteinacous antagonist of PD-1 or a ligand thereof described herein. In a specific embodiment, an antibody or other proteinacous antagonist of PD-1 or a ligand thereof described herein may be prepared, expressed, created or isolated by any means that involves creation, e.g., via synthesis or genetic engineering of sequences. In a specific embodiment, such an antibody or other proteinacous antagonist of PD-1 or a ligand thereof comprises sequences that are encoded by DNA sequences that do not naturally exist within the antibody germline repertoire of an animal or mammal (e.g., a human).

In certain aspects, a method for making an antibody or other proteinacous antagonist of PD-1 or a ligand thereof described herein comprises the step of culturing a cell (e.g., host cell or hybridoma cell) that expresses the antibody or other proteinacous antagonist of PD-1 or a ligand thereof. In certain embodiments, the method for making an antibody or other proteinacous antagonist of PD-1 or a ligand thereof described herein further comprises the step of purifying the antibody or other proteinacous antagonist of PD-1 or a ligand thereof expressed by the cell. In certain aspects, a method for making an antibody or other proteinacous antagonist of PD-1 or a ligand thereof described herein, comprises the step of culturing a cell (e.g., host cell or hybridoma cell) that comprises polynucleotides or vectors encoding the antibody or other proteinacous antagonist of PD-1 or a ligand thereof. In a particular aspect, provided herein are methods for producing an antibody described herein, comprising expressing such antibody from a host cell. The cells may be primary cells or cell lines. In a particular embodiment, the host cell is isolated from other cells. In another embodiment, the host cell is not found within the body of a subject.

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

In certain aspects, provided herein are cells (e.g., host cells) expressing (e.g., recombinantly expressing) the antibodies described herein and related expression vectors. In another aspect, provided herein are vectors (e.g., expression vectors) comprising polynucleotides comprising nucleotide sequences encoding antibodies for recombinant expression in host cells, preferably in mammalian cells. Also provided herein are host cells comprising a polynucleotide encoding an antibody, or vectors comprising a polynucleotide encoding an antibody for recombinantly expressing an antibody described herein. In a specific embodiment, provided herein is a host cell comprising two vectors, wherein the first vector comprises a polynucleotide encoding the variable heavy chain region of an antibody described herein and the second vector comprises a polynucleotide encoding the variable light chain region of an antibody for recombinantly expressing an antibody described herein. The cells may be primary cells or cell lines. In a particular embodiment, the host cell is isolated from other cells. In another embodiment, the host cell is not found within the body of a subject.

Antibodies described herein (e.g., monoclonal antibodies, such as chimeric or humanized antibodies, or an antigen-binding fragment thereof) can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Monoclonal, polyclonal, and humanized antibodies can be prepared by techniques known in the art (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will bind to the protein (e.g., PD-1) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). Additionally, a RIMMS (repetitive immunization multiple sites) technique can be used to immunize an animal (Kilptrack et al., 1997 Hybridoma 16:381-9, incorporated by reference in its entirety).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen of interest (e.g., PD-1). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI 1640 medium. Alternatively, clonal cells can be isolated using a semi-solid agar supplemented with HAT (Stemcell Technologies). In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

In some embodiments, mice (or other animals, such as rats, monkeys, donkeys, pigs, sheep, goats, hamsters, or dogs) can be immunized with an antigen (e.g., human PD-1) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the American Type Culture Collection (ATCC®) (Manassas, VA), to form hybridomas. Hybridomas are selected and cloned by limited dilution. In certain embodiments, lymph nodes of the immunized mice are harvested and fused with NS0 myeloma cells.

The hybridoma clones are then assayed by methods known in the art for cells that secrete antibodies capable of binding a polypeptide of the antigen (e.g., human PD-1). Ascites fluid, which generally contains high levels of antibodies, can be generated by immunizing mice with positive hybridoma clones.

Accordingly, described herein are methods of making antibodies described herein by culturing a hybridoma cell secreting an antibody. In certain embodiments, the method of making an antibody described herein further comprises the step of purifying the antibody.

In specific embodiments, the hybridoma is generated by fusing splenocytes isolated from a mouse (or other animal, such as rat, monkey, donkey, pig, sheep, or dog) immunized with an antigen of interest (e.g., human PD-1) with myeloma cells and then screening the hybridomas resulting from the fusion for hybridoma clones that secrete an antibody able to bind to the antigen. In certain embodiments, the hybridoma is generated by fusing lymph nodes isolated from a mouse (or other animal, such as rat, monkey, donkey, pig, sheep, or dog) immunized with an antigen of interest (e.g., human PD-1) with myeloma cells, and then screening the hybridomas resulting from the fusion for hybridoma clones that secrete an antibody able to bind to the antigen.

Antibodies described herein include antibody fragments which recognize an antigen of interest (e.g., human PD-1) and can be generated by any technique known to those of skill in the art. For example, Fab and F (ab′) 2 fragments described herein can be produced by proteolytic cleavage of immunoglobulin molecules, using enzymes such as papain (to produce Fab fragments) or pepsin (to produce F (ab′) 2 fragments). A Fab fragment corresponds to one of the two identical arms of an antibody molecule and contains the complete light chain paired with the VH and CHI domains of the heavy chain. A F (ab′) 2 fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VHCR and VLCR are amplified from animal cDNA libraries (e.g., human or murine cDNA libraries of affected tissues). The DNA encoding the VHCR and VLCR are recombined together with an scFv linker by PCR and cloned into a phagemid vector. The vector is electroporated in E. coli and the E. coli is infected with helper phage. Phage used in these methods are typically filamentous phage including fd and M13, and the VHCR and VLCR are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F (ab′) 2 fragments can also be employed using methods known in the art such as those disclosed in PCT publication No. WO 92/22324; Mullinax et al., 1992, BioTechniques 12 (6): 864-869; Sawai el al., 1995, AJRI 34:26-34; and Better et al., 1988, Science 240:1041-1043.

In one aspect, to generate whole antibodies, PCR primers including VHCR or VLCR nucleotide sequences, a restriction site, and a flanking sequence to protect the restriction site can be used to amplify the VHCR or VLCR sequences from a template, e.g., scFv clones. Utilizing cloning techniques known to those of skill in the art, the PCR amplified VHCR can be cloned into vectors expressing a variable heavy constant region, and the PCR amplified VLCR can be cloned into vectors expressing a variable light constant region, e.g., human kappa or lambda constant regions. The VHCR and VLCR can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it can be preferable to use human, humanized or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741.

A chimeric antibody is a molecule in which different portions of the antibody are derived from different immunoglobulin molecules. For example, a chimeric antibody can contain a variable region of a mouse monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

In some embodiments, humanized antibodies are produced. A humanized antibody is capable of binding to a predetermined antigen and comprises a framework region having substantially the amino acid sequence of a human immunoglobulin and CDRs having substantially the amino acid sequence of a non-human immunoglobulin (e.g., a murine immunoglobulin). Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592, 106 and EP 519,596; Padlan, 1991, Molecular Immunology 28 (4/5): 489-498; Studnicka et al., 1994, Protein Engineering 7 (6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13 (5): 353-60 (2000), Morea et al., Methods 20 (3): 267 79 (2000), Baca et al., J. Biol. Chem. 272 (16): 10678-84 (1997), Roguska et al., Protein Eng. 9 (10): 895 904 (1996), Couto et al., Cancer Res. 55 (23 Supp): 5973s-5977s (1995), Couto et al., Cancer Res. 55 (8): 1717-22 (1995), Sandhu J S, Gene 150 (2): 409-10 (1994), and Pedersen et al., J. Mol. Biol. 235 (3): 959-73 (1994). See also U.S. Patent Pub. No. US 2005/0042664 A1 (Feb. 24, 2005), which is incorporated by reference herein in its entirety.

Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., human PD-1). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598.

In some embodiments, human antibodies can be produced using mouse human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that bind to a target antigen (e.g., an influenza B virus NA). Such methods are known and are described in the art, see, e.g., Shinmoto et al., Cytotechnology, 2004, 46:19-23; Naganawa et al., Human Antibodies, 2005, 14:27-31.

In some embodiments, human antibodies can be generated by inserting polynucleotides encoding human CDRs (e.g., VLCR CDRs and/or VHCR CDRs) of an antibody into an expression vector containing nucleotide sequences encoding human framework region sequences. In certain embodiments, such expression vectors further comprise nucleotide sequences encoding a constant region of a human light and/or heavy chain. In some embodiments, human antibodies can be generated by inserting human CDRs (e.g., VLCR CDRs and/or VHCR CDRs) of an antibody obtained from a phage library into such human expression vectors.

In certain embodiments, a human antibody can be generated by selecting human CDR sequences that are homologous (or substantially homologous) to non-human CDR sequences of a non-human antibody, and selecting human framework sequences that are homologous (or substantially homologous) to non-human framework sequences of a non-human antibody.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1 (3): 253-263; Muylderman, 2001, J. Biotechnol. 74 (4): 277302; U.S. Pat. No. 6,005,079; and International Publication Nos. WO 94/04678, WO 94/25591, and WO 01/44301.

Bispecific antibodies are antibodies that have binding specificities for at least two different epitopes. Exemplary bispecific antibodies may bind to two different epitopes of an antigen or to two different epitopes of two different antigens. In specific embodiments, a bispecific antibody has two distinct antigen-binding domains, wherein each domain specifically binds to a different antigen. Other such antibodies may bind a first antigen (e.g., human PD-1) and further bind a second antigen. Bispecific antibodies can be prepared as full-length antibodies or antibody fragments (e.g., F (ab′): bispecific antibodies).

Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148 (5): 1547-1553 (1992); Hollinger et al., Proc. Natl. Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731, 168; 4,676,980; and 4,676,980, WO 94/04690; WO 91/00360; WO 92/200373; WO 93/17715; WO 92/08802; and EP 03089.)

Recombinant expression of an antibody described herein (e.g., a full-length antibody, heavy and/or light chain of an antibody, or a single chain antibody described herein) that binds to an antigen of interest (e.g., human PD-1), can for example, involve construction of vectors (e.g., expression vectors) containing a polynucleotide that encodes the antibody or fragments thereof (e.g., VLCR and/or VHCR). Once a polynucleotide encoding an antibody molecule, heavy and/or light chain of an antibody, or antigen-binding fragment thereof described herein has been obtained, a vector for the production of the antibody molecule can be produced by recombinant DNA technology using techniques well-known in the art. Methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and the variable domain of the antibody can be cloned into such a vector for expression of the entire heavy, the entire light chain, or both the entire heavy and light chains.

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16:741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

An expression vector can be transferred to a cell (e.g., host cell) by conventional techniques and the resulting cells can then be cultured by conventional techniques to produce an antibody described herein or a fragment thereof. Thus, provided herein are host cells containing a polynucleotide encoding an antibody described herein or fragments thereof, or a heavy or light chain thereof, or antigen-binding fragment thereof, or a single chain antibody described herein, operably linked to a promoter for expression of such sequences in the host cell. In certain embodiments, e.g., for the expression of double-chained antibodies, vectors encoding both the heavy and light chains individually can be co-expressed in the host cell for expression of the entire immunoglobulin molecule, as detailed below. In certain embodiments, a host cell contains a vector comprising a polynucleotide encoding both the heavy chain and light chain of an antibody described herein, or a fragment thereof. In specific embodiments, a host cell contains two different vectors, a first vector comprising a polynucleotide encoding a heavy chain of an antibody described herein, or a fragment thereof, and a second vector comprising a polynucleotide encoding a light chain of an antibody described herein, or a fragment thereof. In other embodiments, a first host cell comprises a first vector comprising a polynucleotide encoding a heavy chain of an antibody described herein, or a fragment thereof, and a second host cell comprises a second vector comprising a polynucleotide encoding a light chain of an antibody described herein.

A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK 293, NS0, PER.C6, VERO, CRI.7030, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, a mammalian expression vector is pOptiVEC™ or pcDNA3.3. Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In certain embodiments, antibodies described herein are produced by CHO cells or NS0 cells. In a specific embodiment, the expression of nucleotide sequences encoding antibodies described herein (or fragments thereof) which bind to antigen of interest (e.g., human PD-1) is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

As used herein, the term “host cell” refers to any type of cell, e.g., a primary cell or a cell from a cell line. In specific embodiments, the term “host cell” refers a cell transfected with a polynucleotide and the progeny or potential progeny of such a cell. Progeny of such a cell may not be identical to the parent cell transfected with the polynucleotide due to mutations or environmental influences that may occur in succeeding generations or integration of the polynucleotide into the host cell genome.

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT2O and T47D, NS0 (a murine myeloma cell line that does not endogenously produce any immunoglobulin chains), CRL7030 and HsS78Bst cells. In certain embodiments, humanized monoclonal antibodies described herein are produced in mammalian cells, such as CHO cells.

For long-term, high-yield production of recombinant proteins, stable expression is preferred. For example, cell lines which stably express the antibody molecule can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule. Such engineered cell lines can be particularly useful in screening and evaluation of compositions that interact directly or indirectly with the antibody molecule.

A number of selection systems can be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt-or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11 (5): 155-215); and hygro, which confers resistance to hygromycin (Santerre et al., 1984, Gene 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides.

Alternatively, a single vector can be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody (e.g., a monoclonal antibody, such as a humanized or chimeric antibody or an antigen-binding fragment thereof) described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a specific embodiment, antibodies described herein are isolated or purified.

### 5.6 Compositions & Routes of Administration

Encompassed herein is the use of a NDV described herein (e.g., the chimeric NDVs; see, e.g., Sections 5.1, 5.2, and/or 6) in compositions. Also encompassed herein is the use of plasma membrane fragments from NDV infected cells or whole cancer cells infected with NDV in compositions. In a specific embodiment, the compositions are pharmaceutical compositions, such as immunogenic formulations (e.g., vaccine formulations). The compositions may be used in methods of treating cancer.

In one embodiments, a pharmaceutical composition comprises a NDV described herein (e.g., the chimeric NDVs; see, e.g., Sections 5.1, 5.2, and/or 6), in an admixture with a pharmaceutically acceptable carrier. In a specific embodiment, the chimeric NDV comprises a packaged genome, wherein the genomic RNA sequence of the packaged genome is as set forth in SEQ ID NO: 51, 52, or 60. In some embodiments, the pharmaceutical composition further comprises one or more additional prophylactic or therapeutic agents, such as described in Section 5.7.6, infra. In a specific embodiment, a pharmaceutical composition comprises an effective amount of a NDV described herein (e.g., the chimeric NDVs; see, e.g., Sections 5.1, 5.2, and/or 6), and optionally one or more additional prophylactic or therapeutic agents, in a pharmaceutically acceptable carrier. In some embodiments, the NDV (e.g., a chimeric NDV; see, e.g., Sections 5.1, 5.2, and/or 6) is the only active ingredient included in the pharmaceutical composition.

In another embodiment, a pharmaceutical composition (e.g., an oncolysate vaccine) comprises a protein concentrate or a preparation of plasma membrane fragments from NDV infected cancer cells, in an admixture with a pharmaceutically acceptable carrier. In some embodiments, the pharmaceutical composition further comprises one or more additional prophylactic or therapeutic agents, such as described in Section 5.7.6, infra. In another embodiment, a pharmaceutical composition (e.g., a whole cell vaccine) comprises cancer cells infected with NDV, in an admixture with a pharmaceutically acceptable carrier. In some embodiments, the pharmaceutical composition further comprises one or more additional prophylactic or therapeutic agents, such as described in Section 5.7.6, infra.

In another embodiment, a pharmaceutical composition comprises an antagonist of PD-1 or a ligand thereof described herein (see, e.g., Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of an antagonist of PD-1 or a ligand thereof described herein (see, e.g., Section 5.5), and optionally one or more additional prophylactic or therapeutic agents, in a pharmaceutically acceptable carrier. In some embodiments, the antagonist of PD-1 or a ligand thereof (see, e.g., Section 5.5) is the only active ingredient included in the pharmaceutical composition.

In another embodiment, a pharmaceutical composition comprises a PD-1 blocking antibody (see, e.g., Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of a PD-1 blocking antibody described herein (see, e.g., Section 5.5), and optionally one or more additional prophylactic or therapeutic agents, in a pharmaceutically acceptable carrier. In certain embodiments, the pharmaceutical composition further comprises a chimeric NDV described herein (e.g., a chimeric NDV comprising a packaged genome comprising a transgene encoding IL-12 or derivative thereof). In certain embodiments, the pharmaceutical composition further comprises a chimeric NDV described herein (e.g., a chimeric NDV comprising a packaged genome comprising a transgene encoding human IL-12). In some embodiments, the PD-1 blocking antibody (see, e.g., Section 5.5) is the only active ingredient included in the pharmaceutical composition. In a specific embodment, the PD-1 blocking antibody is nivolumab. In a preferred embodiment, the PD-1 blocking antibody is pembrolizumab. In a specific embodiment, the PD-1 blocking comprises: (a) a VLCR CDR1 comprising the amino acid sequence RASKGVSTSGYSYLH (SEQ ID NO: 1), (b) a VLCR CDR2 comprising the amino acid sequence LASYLES (SEQ ID NO: 2), (c) a VLCR CDR3 comprising the amino acid sequence QHSRDLPLT (SEQ ID NO: 3), (d) a VHCR CDR1 comprising the amino acid sequence NYYMY (SEQ ID NO: 6), (e) a VHCR CDR2 comprising the amino acid sequence GINPSNGGTNFNEKFKN (SEQ ID NO: 7), and (f) a VHCR CDR3 comprising the amino acid sequence RDYRFDMGFDY (SEQ ID NO: 8), as determined according to the Kabat numbering system. In another specific embodiment, the PD-1 blocking comprises: (a) a VLCR CDR1 comprising the amino acid sequence RASQSVSSYLA (SEQ ID NO: 11), (b) a VLCR CDR2 comprising the amino acid sequence DASNRAT (SEQ ID NO: 12), (c) a VLCR CDR3 comprising the amino acid sequence QQSSNWPRT (SEQ ID NO: 13), (d) a VHCR CDR1 comprising the amino acid sequence NSGMH (SEQ ID NO: 16), (e) a VHCR CDR2 comprising the amino acid sequence VIWYDGSKRYYADSVKG (SEQ ID NO: 17), and (f) a VHCR CDR3 comprising the amino acid sequence NDDY (SEQ ID NO: 18).

In another embodiment, a pharmaceutical composition comprises a PD-L1 blocking antibody (see, e.g., Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of a PD-L1 blocking antibody described herein (see, e.g., Section 5.5), and optionally one or more additional prophylactic or therapeutic agents, in a pharmaceutically acceptable carrier. In certain embodiments, the pharmaceutical composition further comprises a chimeric NDV described herein (e.g., a chimeric NDV comprising a packaged genome comprising a transgene encoding IL-12 or derivative thereof). In certain embodiments, the pharmaceutical composition further comprises a chimeric NDV described herein (e.g., a chimeric NDV comprising a packaged genome comprising a transgene encoding human IL-12). In some embodiments, the PD-L1 blocking antibody (see, e.g., Section 5.5) is the only active ingredient included in the pharmaceutical composition. In a specific embodiment, the PD-L1 blocking antibody is duralumab or azelumab.

In a specific embodiment of the pharmaceutical composition comprising an antagonist of PD-1 or a ligand thereof, the pharmaceutical composition is formulated as a lyophilized powder or cake. In a specific embodiment, the lyophilized powder or cake is packaged in a single-use vial for reconstitution. In a specific embodiment, the lyophilized powder or cake is formulated in L-histidine, polysorbate 80, and sucrose, and optionally, hydrochloric acid and/or sodium hydroxide to adjust the pH to 5.5. In a specific embodiment, the lyophilized powder or cake is reconstituted with an amount Sterile Water for Injection to achieve the desired concentration of the antagonist of PD-1 or ligand thereof, by, e.g., injecting the Sterile Water for Injection into the vial comprising the lyophilized powder or cake, slowly swirling the vial to allow for reconstitution of the lyophilized powder or cake, and waiting a period of time for the lyophilized powder or cake to be fully reconstituted. In a specific embodiment, 2 mL of reconstituted antagonist contains 50 mg of the antagonist and is formulated in L-histidine (3.1 mg), polysorbate 80 (0.4 mg), and sucrose (140 mg), and optionally, hydrochloric acid and/or sodium hydroxide to adjust pH to 5.5. See, e.g., the Full Prescribing Information for KETRUDA (pembrolizumab), Reference ID: 3862712, which is incorporated by referenced herein in its entirety. Once reconstituted, the pharmaceutical composition should be stored at room temperature for no more than six hours from the time of reconstitution, or under refrigeration at 2 degrees Celsius to 8 degrees Celsius for no more than 24 hours from the time of reconstitution. See, e.g., the Full Prescribing Information for KETRUDA (pembrolizumab), Reference ID: 3862712, which is incorporated by referenced herein in its entirety. In a specific embodiment, the reconstituted pharmaceutical composition is further formulated for intravenous infusion. For example, a desired amount of the reconstituted antagonist of PD-1 or ligand thereof is transferred into a sterile intravenous bag containing, e.g., 0.9% sodium chloride or 5% dextrose in a volume suitable to achieve a desired concentration of the antagonist of PD-1 or ligand thereof.

In another specific embodiment of the pharmaceutical composition comprising an antagonist of PD-1 or a ligand thereof, the pharmaceutical composition is formulated as a liquid solution. In a specific embodiment, the pharmaceutical composition is further formulated for intravenous infusion. For example, a desired amount of the pharmaceutical composition is transferred into a sterile intravenous bag containing, e.g., 0.9% sodium chloride or 5% dextrose in a volume suitable to achieve a desired concentration of the antagonist of PD-1 or ligand thereof. See, e.g., the Full Prescribing Information for OPDIVO (nivolumab), Reference ID: 3677021, which is incorporated by referenced herein in its entirety.

In another embodiment, a pharmaceutical composition comprises (i) an NDV described herein (see, e.g., Sections 5.1, 5.2, and/or 6), and (ii) an antagonist of PD-1 or a ligand thereof described herein (see, e.g., Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of (i) an NDV described herein (see, e.g., Sections 5.1, 5.2, and/or 6), and (ii) an antagonist of PD-1 or a ligand thereof described herein (see, e.g., Section 5.5), and optionally one or more additional prophylactic or therapeutic agents, in a pharmaceutically acceptable carrier. In some embodiments, the NDV (see, e.g., Sections 5.1, 5.2, and/or 6) and the antagonist of PD-1 or a ligand thereof are the only active ingredients included in the pharmaceutical composition.

In another embodiment, a pharmaceutical composition comprises (i) a chimeric NDV comprising a packaged genome comprising a transgene encoding IL-12 (e.g., human IL-12) or a derivative thereof, and (ii) an antagonist of PD-1 or a ligand thereof described herein (see, e.g., Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of (i) an NDV described herein (see, e.g., Sections 5.1, 5.2, and/or 6), and (ii) an antagonist of PD-1 or a ligand thereof described herein (see, e.g., Section 5.5), and optionally one or more additional prophylactic or therapeutic agents, in a pharmaceutically acceptable carrier. In a specific embodiment, a pharmaceutical composition comprises an effective amount of (i) a chimeric NDV comprising a packaged genome comprising a transgene encoding IL-12 (e.g., human IL-12) or a derivative thereof, and (ii) an antagonist of PD-1 or a ligand thereof described herein (see, e.g., Section 5.5), and optionally one or more additional prophylactic or therapeutic agents, in a pharmaceutically acceptable carrier. In some embodiments, the NDV (see, e.g., Sections 5.1, 5.2, and/or 6) and the antagonist of PD-1 or a ligand thereof are the only active ingredients included in the pharmaceutical composition. In specific embodiments, the chimeric NDV comprising a packaged genome comprising a transgene encoding IL-12 (e.g., human IL-12) or a derivative thereof and the antagonist of PD-1 or a ligand thereof are the only active ingredients included in the pharmaceutical composition.

In another embodiment, a pharmaceutical composition comprises (i) a chimeric NDV comprising a packaged genome comprising a transgene encoding IL-12 (e.g., human IL-12) or a derivative thereof, and (ii) a PD-1 blocking antibody (such as described, e.g., in Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of (i) an NDV described herein (see, e.g., Sections 5.1, 5.2, and/or 6), and (ii) a PD-1 blocking antibody described herein (see, e.g., Section 5.5), and optionally one or more additional prophylactic or therapeutic agents, in a pharmaceutically acceptable carrier. In a specific embodiment, a pharmaceutical composition comprises an effective amount of (i) a chimeric NDV comprising a packaged genome comprising a transgene encoding IL-12 (e.g., human IL-12) or a derivative thereof, and (ii) a PD-1 blocking antibody described herein (see, e.g., Section 5.5), and optionally one or more additional prophylactic or therapeutic agents, in a pharmaceutically acceptable carrier. In some embodiments, the NDV (see, e.g., Sections 5.1, 5.2, and/or 6) and the antagonist of PD-1 or a ligand thereof are the only active ingredients included in the pharmaceutical composition. In specific embodiments, the chimeric NDV comprising a packaged genome comprising a transgene encoding IL-12 (e.g., human IL-12) or a derivative thereof and the PD-1 blocking antibody are the only active ingredients included in the pharmaceutical composition. In a specific embodiment, the PD-1 blocking antibody is nivolumab. In a preferred embodiment, the PD-1 blocking antibody is pembrolizumab. In a specific embodiment, the pharmaceutical composition comprises: (i) an antibody comprising: (a) a VLCR CDR1 comprising the amino acid sequence RASKGVSTSGYSYLH (SEQ ID NO: 1), (b) a VLCR CDR2 comprising the amino acid sequence LASYLES (SEQ ID NO: 2), (c) a VLCR CDR3 comprising the amino acid sequence QHSRDLPLT (SEQ ID NO: 3), (d) a VHICR CDR1 comprising the amino acid sequence NYYMY (SEQ ID NO: 6), (e) a VHICR CDR2 comprising the amino acid sequence GINPSNGGTNFNEKFKN (SEQ ID NO: 7), and (f) a VHCR CDR3 comprising the amino acid sequence RDYRFDMGFDY (SEQ ID NO: 8), as determined according to the Kabat numbering system; and (ii) a chimeric NDV comprising a packaged genome comprising the nucleotide sequence set forth in SEQ ID NO: 51. In a specific embodiment, the pharmaceutical composition comprises: (i) an antibody comprising: (a) a VLCR CDR1 comprising the amino acid sequence RASKGVSTSGYSYLH (SEQ ID NO: 1), (b) a VLCR CDR2 comprising the amino acid sequence LASYLES (SEQ ID NO: 2), (c) a VLCR CDR3 comprising the amino acid sequence QHSRDLPLT (SEQ ID NO: 3), (d) a VHCR CDR1 comprising the amino acid sequence NYYMY (SEQ ID NO: 6), (e) a VHCR CDR2 comprising the amino acid sequence GINPSNGGTNFNEKFKN (SEQ ID NO: 7), and (f) a VHCR CDR3 comprising the amino acid sequence RDYRFDMGFDY (SEQ ID NO: 8), as determined according to the Kabat numbering system; and (ii) a chimeric NDV comprising a packaged genome comprising the nucleotide sequence set forth in SEQ ID NO: 52. In a specific embodiment, the pharmaceutical composition comprises: (i) an antibody comprising: (a) a VLCR CDR1 comprising the amino acid sequence RASKGVSTSGYSYLII (SEQ ID NO: 1), (b) a VLCR CDR2 comprising the amino acid sequence LASYLES (SEQ ID NO: 2), (c) a VLCR CDR3 comprising the amino acid sequence QHSRDLPLT (SEQ ID NO: 3), (d) a VHCR CDR1 comprising the amino acid sequence NYYMY (SEQ ID NO: 6), (e) a VHCR CDR2 comprising the amino acid sequence GINPSNGGTNFNEKFKN (SEQ ID NO: 7), and (f) a VHCR CDR3 comprising the amino acid sequence RDYRFDMGFDY (SEQ ID NO: 8), as determined according to the Kabat numbering system; and (ii) a chimeric NDV comprising a packaged genome comprising the nucleotide sequence set forth in SEQ ID NO: 60.

In another embodiment, a pharmaceutical composition comprises (i) a chimeric NDV comprising a packaged genome comprising a transgene encoding IL-12 (e.g., human IL-12) or a derivative thereof, and (ii) a PD-L1 blocking antibody (such as described, e.g., in Section 5.5), in an admixture with a pharmaceutically acceptable carrier. In some embodiments, the pharmaceutical composition further comprises one or more additional prophylactic or therapeutic agents, such as described in Section 5.7.6, infra. In a specific embodiment, a pharmaceutical composition comprises an effective amount of (i) an NDV described herein (see, e.g., Sections 5.1, 5.2, and/or 6), and (ii) a PD-L1 blocking antibody described herein (see, e.g., Section 5.5), and optionally one or more additional prophylactic or therapeutic agents, in a pharmaceutically acceptable carrier. In a specific embodiment, a pharmaceutical composition comprises an effective amount of (i) a chimeric NDV comprising a packaged genome comprising a transgene encoding IL-12 (e.g., human IL-12) or a derivative thereof, and (ii) a PD-L1 blocking antibody described herein (see, e.g., Section 5.5), and optionally one or more additional prophylactic or therapeutic agents, in a pharmaceutically acceptable carrier. In some embodiments, the NDV (see, e.g., Sections 5.1, 5.2, and/or 6) and the antagonist of PD-L1 or a ligand thereof are the only active ingredients included in the pharmaceutical composition. In specific embodiments, the chimeric NDV comprising a packaged genome comprising a transgene encoding IL-12 (e.g., human IL-12) or a derivative thereof and the PD-L1 blocking antibody are the only active ingredients included in the pharmaceutical composition. In a specific embodiment, the PD-L1 blocking antibody is duralumab or avclumab.

The pharmaceutical compositions provided herein can be in any form that allows for the composition to be administered to a subject. In a specific embodiment, the pharmaceutical compositions are suitable for veterinary and/or human administration. As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, the pharmaceutical compositions are formulated to be suitable for the intended route of administration to a subject. For example, the pharmaceutical composition may be formulated to be suitable for parenteral, intravenous, intraarterial, intrapleural, inhalation, intraperitoneal, oral, intradermal, colorectal, intraperitoneal, intracranial, and intratumoral administration. In a specific embodiment, the pharmaceutical composition may be formulated for intravenous, intraarterial, oral, intraperitoneal, intranasal, intratracheal, intrapleural, intracranial, subcutaneous, intramuscular, topical, pulmonary, or intratumoral administration.

In a specific embodiment, the pharmaceutical composition comprising an NDV described herein (see, e.g., Sections 5.1, 5.2, and/or 6) is formulated to be suitable for intratumoral administration to the subject (e.g., human subject). In a specific embodiment, the pharmaceutical composition comprising a chimeric NDV comprising a packaged genome comprising a transgene encoding IL-12 (e.g., human IL-12) or a derivative thereof is formulated to be suitable for intratumoral administration to the subject (e.g., human subject). In a specific embodiment, the sequence of the packaged genome comprising a transgene encoding IL-12 comprises or consists of the sequence set forth in SEQ ID NO: 51. In a specific embodiment, the sequence of the packaged genome comprising a transgene encoding IL-12 comprises or consists of the sequence set forth in SEQ ID NO: 52. In a specific embodiment, the sequence of the packaged genome comprising a transgene encoding IL-12 comprises or consists of the sequence set forth in SEQ ID NO: 60.

In a specific embodiment, the pharmaceutical composition comprising an NDV described herein (see, e.g., Sections 5.1, 5.2, and/or 6) is formulated to be suitable for intravenous administration to the subject (e.g., human subject). In a specific embodiment, the pharmaceutical composition comprising a chimeric NDV comprising a packaged genome comprising a transgene encoding IL-12 (e.g., human IL-12) or a derivative thereof is formulated to be suitable for intravenous administration to the subject (e.g., human subject). In a specific embodiment, the sequence of the packaged genome comprising a transgene encoding IL-12 is as set forth in SEQ ID NO: 51. In a specific embodiment, the sequence of the packaged genome comprising a transgene encoding IL-12 is as set forth in SEQ ID NO: 52. In a specific embodiment, the sequence of the packaged genome comprising a transgene encoding IL-12 is as set forth in SEQ ID NO: 60.

In a specific embodiment, the pharmaceutical composition comprising an antagonist of PD-1 or a ligand thereof described herein (see, e.g., Section 5.5) is formulated to be suitable for intravenous administration to the subject (e.g., human subject). In a specific embodiment, the pharmaceutical composition comprising a PD-1 blocking antibody described herein (see, e.g., Section 5.5) is formulated to be suitable for intravenous administration to the subject (e.g., human subject). In a specific embodiment, the PD-1 blocking antibody is nivolumab. In a preferred embodiment, the PD-1 blocking antibody is pembrolizumab. In a specific embodiment, the pharmaceutical composition comprising a PD-L1 blocking antibody described herein (see, e.g., Section 5.5) is formulated to be suitable for intravenous administration to the subject (e.g., human subject). In a specific embodiment, the PD-L1 blocking antibody is duralumab or avelumab.

### 5.7 Anti-Cancer Uses and Other Uses

### 5.7.1 Methods of Treating Cancer with NDV-IL12 and an Antagonist of PD-1 or A Ligand Thereof

In one aspect, presented herein are methods for treating cancer utilizing a chimeric NDV described herein (e.g., a chimeric NDV described in Section 5.2 and/or Section 6) or a composition comprising such a chimeric NDV in combination with an antagonist of PD-1 or a ligand thereof (e.g., an antagonist described in Section 5.5 and/or Section 6) or composition comprising such an antagonist, wherein the chimeric NDV comprises a packaged genome which encodes IL-12 or a derivative thereof. In a specific embodiment, the IL-12 or derivative thereof is expressed by cells infected with the chimeric NDV. The chimeric NDV, or a composition thereof, and the antagonist of PD-1 or a ligand thereof, or a composition thereof, may be used as any line of therapy (e.g., a first, second, third, fourth or fifth line therapy). In a specific embodiment, the method of treatment further comprises administering to the subject one or more additional therapies described in Section 5.7.6, e.g., Section 5.7.6.1.

In one embodiment, presented herein are methods for treating cancer comprising administering to a subject a chimeric NDV and an antagonist of PD-1 or a ligand thereof, wherein the chimeric NDV comprises a packaged genome comprising a transgene encoding IL-12 or a derivative thereof (e.g., human IL-12). In a specific embodiment, the IL-12 or derivative thereof is expressed by cells infected with the chimeric NDV. In another embodiment, presented herein are methods for treating cancer comprising administering to a subject an effective amount of a chimeric NDV and an effective amount of an antagonist of PD-1 or a ligand thereof, wherein the chimeric NDV comprises a packaged genome comprising a transgene encoding IL-12 or a derivative thereof (e.g., human IL-12). In a specific embodiment, the IL-12 or derivative thereof is expressed by cells infected with the chimeric NDV. The chimeric NDV and antagonist may be administered concurrently or sequentially to the subject. In certain embodiments, the chimeric NDV and antagonist are administered in the same composition. In other embodiments, the chimeric NDV and antagonist are administered in different compositions. The chimeric NDV and antagonist may be administered by the same or different routes of administration to the subject. Any route known to one of skill in the art or described herein may be used to administer the chimeric NDV and antagonist. In a specific embodiment, the chimeric NDV is administered intratumorally and the antagonist is administered intravenously. In some embodiments, the chimeric NDV and the antagonist are administered intravenously.

In another aspect, presented herein are uses of a chimeric NDV in the preparation of a medicament for use in combination with an antagonist of PD-1 or a ligand thereof for treating cancer in a subject (e.g., a human subject), wherein the chimeric NDV comprises a packaged genome comprising a transgene encoding IL-12 or a derivative thereof (e.g., human IL-12). In another aspect, presented herein is a chimeric NDV for use in a method for treating cancer in a subject (e.g., a human subject), wherein the chimeric NDV comprises a packaged genome comprising a transgene encoding IL-12 or a derivative thereof, and wherein the method further comprising administering an antagonist of PD-1 or a ligand thereof (e.g., human IL-12). In a specific embodiment, the IL-12 or derivative thereof is expressed by cells infected with the chimeric NDV. The chimeric NDV and antagonist may be administered concurrently or sequentially to the subject. In certain embodiments, the chimeric NDV and antagonist are administered in the same composition. In other embodiments, the chimeric NDV and antagonist are administered in different compositions. The chimeric NDV and antagonist may be administered by the same or different routes of administration to the subject. Any route known to one of skill in the art or described herein may be used to administer the chimeric NDV and antagonist. In a specific embodiment, the chimeric NDV is administered intratumorally and the antagonist is administered intravenously. In some embodiments, the chimeric NDV and the antagonist are administered intravenously. In another embodiment, the chimeric NDV is administered intra-nodally and the antagonist are administered intravenously.

In another embodiment, presented herein is a method for treating cancer, comprising administering to a subject (e.g., a human subject) in need thereof a first composition comprising a chimeric NDV and a second composition comprising an antagonist of PD-1 or a ligand thereof, wherein the chimeric NDV comprises a packaged genome comprising a transgene encoding IL-12 (e.g., human IL-12), wherein the transgene encodes an IL-12 p40 subunit and an IL-12 p35 subunit. In another embodiment, presented herein is a method for treating cancer, comprising administering to a subject (e.g., a human subject) in need thereof a chimeric NDV and an antagonist of PD-1 or a ligand thereof, wherein the chimeric NDV comprises a packaged genome which encodes IL-12, and wherein the antagonist of PD-1 is an antibody that binds to PD-1 and blocks (completely or partially) the interaction between PD-1 and a ligand thereof (e.g., either PD-L1, PD-L2, or both) (sometimes referred herein as a “PD-1 blocking antibody”). In another embodiment, presented herein is a method for treating cancer, comprising administering to a subject (e.g., a human subject) in need thereof a first composition comprising a chimeric NDV and a second composition comprising a PD-1-blocking antibody, wherein the chimeric NDV comprises a packaged genome comprising a transgene encoding IL-12 (e.g., human IL-12), wherein the transgene encodes an IL-12 p40 subunit and an IL-12 p35 subunit. In another embodiment, presented herein is a method for treating cancer, comprising administering to a human subject in need thereof a first composition comprising a chimeric Newcastle disease virus (NDV) and a second composition comprising a PD-1-blocking antibody, wherein the chimeric NDV comprises a packaged genome comprising a transgene encoding human IL-12, wherein the transgene encodes a human IL-12 p40 subunit and a human IL-12 p35 subunit. In a specific embodiment, the sequence of the packaged genome comprising a transgene encoding human IL-12 is as set forth in SEQ ID NO: 51. In a specific embodiment, the sequence of the packaged genome comprising a transgene encoding human IL-12 is as set forth in SEQ ID NO: 52. In a specific embodiment, the sequence of the packaged genome comprising a transgene encoding human IL-12 is as set forth in SEQ ID NO: 60. In a preferred embodiment, the PD-1 blocking antibody is pembrolizumab. In other embodiments, the PD-1 blocking antibody is nivolumab, MEDI0680, PDR001, or atezolizumab. The first and second compositions may be administered by same or different routes of administration. Any route known to one of skill in the art or described herein may used to administer the first and second compositions. In a specific embodiment, the first composition is administered intratumorally and the second composition is administered intravenously. In some embodiments, the first and second compositions are administered intravaneously. See, e.g., Sections 5.1 and 5.2, supra, and Section 6, infra, for information regarding NDV, Section 5.5, supra, and Section 6, infra, for information regarding antagonists of PD-1 or a ligand thereof, and Section 5.5.1, supra, for information regarding compositions and routes of administration.

In another embodiment, presented herein is a use of a chimeric NDV in the preparation of a medicament for use in combination with an antagonist of PD-1 or a ligand thereof for treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit. In another embodiment, presented herein is a use of a chimeric NDV in the preparation of a medicament for use in combination with an antagonist of PD-1 or a ligand thereof for treating cancer in a subject (e.g., a human subject), wherein the chimeric NDV comprises a packaged genome which encodes IL-12 (e.g., human IL-12), and wherein the antagonist of PD-1 is an antibody that binds to PD-1 (e.g., human PD-1) and blocks (completely or partially) the interaction between PD-1 and a ligand thereof (e.g., either PD-L1, PD-L2, or both) (sometimes referred herein as a “PD-1 blocking antibody”). In another embodiment, presented herein is a use of a chimeric NDV in the preparation of a medicament for use in combination with a PD-1 blocking antibody for treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit. In another embodiment, presented herein is a use of a chimeric NDV in the preparation of a medicament for use in combination with a PD-1 blocking antibody for treating cancer in a subject (e.g., a human subject), wherein the chimeric NDV comprises a packaged genome comprising a transgene encoding human IL-12, wherein the transgene encodes a human IL-12 p40 subunit and a human IL-12 p35 subunit. In a specific embodiment, the sequence of the packaged genome comprising a transgene encoding IL-12 is as set forth in SEQ ID NO: 51. In a specific embodiment, the sequence of the packaged genome comprising a transgene encoding IL-12 is as set forth in SEQ ID NO: 52. In a specific embodiment, the sequence of the packaged genome comprising a transgene encoding IL-12 is as set forth in SEQ ID NO: 60. In a specific embodiment, the transgene encodes an IL-12 amino acid sequence with sequence set forth in Table 7. In a specific embodiment, the transgene encodes an IL-12 amino acid sequence comprising an amino acid sequence set forth in Table 7. In a specific embodiment, the transgene encoding IL-12 comprising a nucleotide sequence of a sequence set forth in Table 8. In a specific embodiment, the transgene encoding IL-12 consists of a seqeucne set forth in Table 8. In a preferred embodiment, the PD-1 blocking antibody is pembrolizumab. In other embodiments, the PD-1 blocking antibody is nivolumab, MEDI0680, PDR001, or atezolizumab. The first and second compositions (i.e., the chimeric NDV and the antagonist) may be administered by same or different routes of administration. Any route known to one of skill in the art or described herein may be used to administer the first and second compositions (i.e., the chimeric NDV and the antagonist). In a specific embodiment, the first composition (i.e., the chimeric NDV) is administered intratumorally and the second composition (i.e., the antagonist) is administered intravenously. In another specific embodiment, the first composition (i.e., the chimeric NDV) is administered intra-nodally and the second composition (i.e., the antagonist) is administered intravenously. In some embodiments, the first and second compositions (i.e., the chimeric NDV and the antagonist) are administered intravenously. See, e.g., Sections 5.1 and 5.2, supra, and Section 6, infra, for information regarding NDV, Section 5.5, supra, and Section 6, infra, for information regarding antagonists of PD-1 or a ligand thereof, and Section 5.5.1, supra, for information regarding compositions and routes of administration.

In another embodiment, presented herein is a chimeric NDV for use in method for treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit, and wherein the method further comprises administering an antagonist of PD-1 or a ligand thereof. In another embodiment, presented herein is a chimeric NDV for use in method for treating cancer in a subject (e.g., a human subject), wherein the chimeric NDV comprises a packaged genome comprising a transgene encoding IL-12 (e.g., human IL-12), wherein the transgene encodes an IL-12 p40 subunit and an IL-12 p35 subunit, wherein the method further comprises administering an antagonist of PD-1 or a ligand thereof, and wherein the antagonist of PD-1 is an antibody that binds to PD-1 (e.g., human PD-1) and blocks (completely or partially) the interaction between PD-1 and a ligand thereof (e.g., either PD-L1, PD-L2, or both) (sometimes referred herein as a “PD-1 blocking antibody”). In another embodiment, presented herein is a chimeric NDV for use in method for treating cancer in a subject (e.g., a human subject), wherein the chimeric NDV comprises a packaged genome comprising a transgene encoding IL-12 (e.g.e.g., human IL-12), wherein the transgene encodes an IL-12 p40 subunit and an IL-12 p35 subunit, and wherein the method further comprises administering PD-1 blocking antibody. In another embodiment, presented herein is a chimeric NDV for use in method for treating cancer in a subject (e.g., a human subject), wherein the chimeric NDV comprises a packaged genome comprising a transgene encoding human IL-12, wherein the transgene encodes a human IL-12 p40 subunit and a human IL-12 p35 subunit, and wherein the method further comprises administering PD-1 blocking antibody. In a specific embodiment, the sequence of the packaged genome comprising a transgene encoding IL-12 is as set forth in SEQ ID NO: 51. In a specific embodiment, the sequence of the packaged genome comprising a transgene encoding IL-12 is as set forth in SEQ ID NO: 52. In a specific embodiment, the sequence of the packaged genome comprising a transgene encoding IL-12 is as set forth in SEQ ID NO: 60. In a preferred embodiment, the PD-1 blocking antibody is pembrolizumab. In other embodiments, the PD-1 blocking antibody is nivolumab, PDR001, MEDI0680, or atezolizumab. The first and second compositions (i.e., the chimeric NDV and the antagonist) may be administered by same or different routes of administration. Any route known to one of skill in the art or described herein may be used to administer the first and second compositions (i.e., the chimeric NDV and the antagonist). In a specific embodiment, the first composition (i.e., the chimeric NDV) is administered intratumorally and the second composition (i.e., the antagonist) is administered intravenously. In another specific embodiment, the first composition (i.e., the chimeric NDV) is administered intra-nodally and the second composition (i.e., the antagonist) is administered intravenously. In some embodiments, the first and second compositions (i.e., the chimeric NDV and the antagonist) are administered intravenously. See, e.g., Sections 5.1 and 5.2, supra, and Section 6, infra, for information regarding NDV, Section 5.5, supra, and Section 6, infra, for information regarding antagonists of PD-1 or a ligand thereof, and Section 5.5.1, supra, for information regarding compositions and routes of administration.

In a specific embodiment, the chimeric NDV comprises a packaged genome comprising a transgene encoding a human IL-12 transgene, wherein the transgene encodes an IL-12 p40 subunit and an IL-12 p35 subunit. See, e.g., Sections 5.2.1, 5.7, and 6 for exmaples of IL-12 trangenes. In a specific embodiment, the chimeric NDV comprises an NDV backbone of LaSota strain. In a specific embodiment, the chimeric NDV comprises an NDV backbone which is lentogenic. In a specific embodiment, the packaged genome comprises a nucleotide sequence encoding a mutated F protein and the mutated F protein is expressed by the chimeric NDV, wherein the mutated F protein comprises a mutated cleavage site. In a specific embodiment, the packaged genome comprises a nucleotide sequence encoding a mutated F protein with the amino acid mutation L289A (i.e., an L to A mutation at the amino acid position corresponding to L289 of the NDV La Sota strain F protein), wherein the mutated F protein is expressed by the chimeric NDV. In a specific embodiment, the chimeric NDV comprises an NDV backbone which is LaSota strain, and wherein the packaged genome encodes a mutated F protein with the amino acid mutation L289A (i.e., an L to A mutation at the amino acid position corresponding to L289 of the NDV La Sota strain F protein), wherein the mutated F protein is expressed by the chimeric NDV. See, e.g., Sections 5.2 and 6 for examples of chimeric NDV encoding IL-12 or a derivative thereof. In a specific embodiment, the chimeric NDV comprises a packaged genome having the nucleotide sequence set forth in SEQ ID NO: 51, 52 or 60.

In a specific embodiment, the antagonist of PD-1 or a ligand thereof is an antagonist described in Sections 5.5 and/or 6. In a specific embodiment, the antagonist of PD-1 or a ligand thereof is an antibody (or an antigen-binding fragment) or a soluble receptor that specifically binds to a ligand of PD-1. In certain embodiments, the soluble receptor is a fragment of PD-1 or a fragment of a derivative of PD-1 that specifically binds to a ligand of PD-1 (e.g., the extracellular domain of PD-1 or a derivative of PD-1). In some embodiments, the soluble receptor is a fusion protein comprising at least a portion of PD-1 or a derivative of PD-1 (e.g., the extracellular domain of PD-1 or a derivative of PD-1), and a heterologous amino acid sequence. In specific embodiments, the fusion protein comprises at least a portion of PD-1 or a derivative of PD-1, and the Fc portion of an immunoglobulin or a fragment thereof. In specific embodiments, the antagonist of PD-1 or a ligand thereof is an antibody (or an antigen-binding fragment) that specifically binds to a ligand of PD-1. In another embodiment, the antagonist of PD-1 or a ligand thereof is an antibody (or an antigen-binding fragment) or ligand that binds to PD-1, but does not transduce an inhibitory signal(s). In another embodiment, the antagonist of PD-1 or a ligand thereof is a ligand that binds to PD-1, but does not transduce an inhibitory signal(s). In certain specific embodiments, the ligand is a fusion protein comprising at least a portion of a ligand of PD-1 or a derivative of a ligand of PD-1, and a heterologous amino acid sequence. In specific embodiments, the fusion protein comprises at least a portion of a ligand of PD-1 or a derivative of a ligand of PD-1, and the Fc portion of an immunoglobulin or a fragment thereof. Nonlimiting examples of antagonists of PD-1 or a ligand thereof include pembrolizumab (“KEYTRUDA®”; see, e.g., Hamid et al., N Engl J Med. 2013; 369:134-44 and Full Prescribing Information for KEYTRUDA (pembrolizumab), Reference ID: 3862712), nivolumab (“OPDIVOR”; see, e.g., Topalian et al., N Engl J Med. 2012; 366:2443-54 and Full Prescribing Information for OPDIVO (nivolumab), Reference ID: 3677021), AMP-224 (see, e.g., Infante et al., J Clin Oncol. 2013; 31 (suppl): abstr 3044), MEDI0680 (also referred to as “AMP-514”; see, e.g., Hamid et al., Ann Oncol. 2016; 27 (suppl_6): 1050PD), durvalumab (also referred to as “medi-4736”; see, e.g., Lutzky et al., J Clin Oncol. 2014; 32 (suppl 5S): abstr 3001), avelumab (e.g., for Merkel cell carcinoma) (also referred to as “MSB0010718C”; see, e.g., Heery et al. J Clin Oncol. 2014; 32 (suppl 5S): abstr 3064), bms-936559 (see, e.g., Brahmer et al. N. Engl. J. Med. 2012; 366, 2455-2465), and atezolizumab (also referred to as “mpd13280A” and “TECENTRIQ®”; see, e.g., McDermott et al., J Clin Oncol. 2016; 34 (8): 833-842, Herbst et al., J Clin Oncol. 2013; 31 (suppl): abstr 3000, and Full Prescribing Information for TECENTRIQ, Reference ID: 3933242). In a specific embodiment, the antagonist of PD-1 or a ligand thereof is a therapy approved by the U.S. FDA for treatment of one or more cancers. Nonlimiting examples of an antagonist of PD-1 or a ligand thereof approved by the U.S. FDA for treatment of cancer include pembrolizumab, nivolumab, atezolizumab, and avelumab. In a specific embodiment, the antagonist of PD-1 or a ligand thereof is a therapy approved by the EMA for treatment of one or more cancers. Nonlimiting examples of an antagonist of PD-1 or a ligand thereof approved by the EMA for treatment of cancer include pembrolizumab, nivolumab, and atezolizumab. In a specific embodiment, the antagonist of PD-1 or a ligand thereof is nivolumab, MEDI0680, or pembrolizumab. In a preferred embodiment, the antagonist of PD-1 or a ligand thereof is pembrolizumab. In another embodiment, the antagonist of PD-1 or a ligand thereof is nivolumab, AMP-224, MEDI0680, PDR001, durvalumab, avelumab, bms-936559, or atezolizumab.

In a specific embodiment, the IL-12 transgene encodes an amino acid sequence set forth in SEQ ID NO: 42. In a specific embodiment, the nucleotide sequence encoding the IL-12 transgene comprises the nucleotide sequence set forth in SEQ ID NO: 53. In a specific embodiment, the transgene is inserted between two transcription units of the packaged genome. In a specific embodiment, the transgene is inserted between two transcription units of the packaged genome, wherein the two transcription units of the packaged genome are the transcription units for the NDV P gene and the NDV M gene. In a specific embodiment, the IL-12 p40 subunit comprises the amino acid sequence set forth in SEQ ID NO: 40. In a specific embodiment, the nucleotide sequence encoding the IL-12 p40 subunit comprises the nucleotide sequence set forth in SEQ ID NO: 54. In a specific embodiment, the IL-12 p35 subunit comprises the amino acid sequence set forth in SEQ ID NO: 41. In a specific embodiment, the nucleotide sequence encoding the IL-12 p35 subunit comprises the nucleotide sequence set forth in SEQ ID NO: 55.

In a specific embodiment, the IL-12 transgene encodes an amino acid sequence comprising the amino acid sequence set forth in SEQ ID NO: 43. In a specific embodiment, the nucleotide sequence encoding the IL-12 transgene comprises the nucleotide sequence set forth in SEQ ID NO: 63. In a specific embodiment, the transgene is inserted between two transcription units of the packaged genome. In a specific embodiment, the transgene is inserted between two transcription units of the packaged genome, wherein the two transcription units of the packaged genome are the transcription units for the NDV P gene and the NDV M gene. In a specific embodiment, the IL-12 p40 subunit comprises the amino acid sequence set forth in SEQ ID NO: 38. In a specific embodiment, the nucleotide sequence encoding the IL-12 p40 subunit comprises the nucleotide sequence set forth in SEQ ID NO: 59. In a specific embodiment, the IL-12 p35 subunit comprises the amino acid sequence set forth in SEQ ID NO: 41. In a specific embodiment, the nucleotide sequence encoding the IL-12 p35 subunit is as set forth in SEQ ID NO: 55.

In a specific embodiment, the IL-12 transgene encodes an amino acid sequence set forth in SEQ ID NO: 22. In a specific embodiment, the nucleotide sequence encoding the IL-12 transgene is as set forth in SEQ ID NO: 26. In a specific embodiment, the transgene is inserted between two transcription units of the packaged genome. In a specific embodiment, the transgene is inserted between two transcription units of the packaged genome, wherein the two transcription units of the packaged genome are the transcription units for the NDV P gene and the NDV M gene. In a specific embodiment, the IL-12 p40 subunit comprises the amino acid sequence set forth in SEQ ID NO: 23. In a specific embodiment, the nucleotide sequence encoding the IL-12 p40 subunit comprises the nucleotide sequence set forth in SEQ ID NO: 27. In a specific embodiment, the IL-12 p35 subunit comprises the amino acid sequence set forth in SEQ ID NO: 25. In a specific embodiment, the nucleotide sequence encoding the IL-12 p35 subunit is as set forth in SEQ ID NO: 29.

In a specific embodiment, the IL-12 transgene encodes an amino acid sequence comprising the amino acid sequence set forth in SEQ ID NO: 39. In a specific embodiment, the nucleotide sequence encoding the IL-12 transgene comprises the nucleotide sequence set forth in SEQ ID NO: 61. In a specific embodiment, the transgene is inserted between two transcription units of the packaged genome. In a specific embodiment, the transgene is inserted between two transcription units of the packaged genome, wherein the two transcription units of the packaged genome are the transcription units for the NDV P gene and the NDV M gene. In a specific embodiment, the IL-12 p40 subunit comprises the amino acid sequence set forth in SEQ ID NO: 38. In a specific embodiment, the nucleotide sequence encoding the IL-12 p40 subunit comprises the nucleotide sequence set forth in SEQ ID NO: 57. In a specific embodiment, the IL-12 p35 subunit comprises the amino acid sequence set forth in SEQ ID NO: 25. In a specific embodiment, the nucleotide sequence encoding the IL-12 p35 subunit comprises the nucleotide sequence set forth in SEQ ID NO: 29.

In a specific embodiment, the IL-12 transgene encodes an amino acid sequence comprising the amino acid sequence set forth in SEQ ID NO: 42. In a specific embodiment, the nucleotide sequence encoding the IL-12 transgene comprises the nucleotide sequence set forth in SEQ ID NO: 66. In a specific embodiment, the transgene is inserted between two transcription units of the packaged genome. In a specific embodiment, the transgene is inserted between two transcription units of the packaged genome, wherein the two transcription units of the packaged genome are the transcription units for the NDV P gene and the NDV M gene. In a specific embodiment, the IL-12 p40 subunit comprises the amino acid sequence set forth in SEQ ID NO: 40. In a specific embodiment, the nucleotide sequence encoding the IL-12 p40 subunit comprises the nucleotide sequence set forth in SEQ ID NO: 64. In a specific embodiment, the IL-12 p35 subunit comprises the amino acid sequence set forth in SEQ ID NO: 41. In a specific embodiment, the nucleotide sequence encoding the IL-12 p35 subunit comprises the nucleotide sequence set forth in SEQ ID NO: 65.

In a specific embodiment, the IL-12 transgene encodes an amino acid sequence comprising the amino acid sequence set forth in SEQ ID NO: 43. In a specific embodiment, the nucleotide sequence encoding the IL-12 transgene comprises the nucleotide sequence set forth in SEQ ID NO: 68. In a specific embodiment, the transgene is inserted between two transcription units of the packaged genome. In a specific embodiment, the transgene is inserted between two transcription units of the packaged genome, wherein the two transcription units of the packaged genome are the transcription units for the NDV P gene and the NDV M gene. In a specific embodiment, the IL-12 p40 subunit comprises the amino acid sequence set forth in SEQ ID NO: 38. In a specific embodiment, the nucleotide sequence encoding the IL-12 p40 subunit comprises the nucleotide sequence set forth in SEQ ID NO: 57. In a specific embodiment, the IL-12 p35 subunit comprises the amino acid sequence set forth in SEQ ID NO: 41. In a specific embodiment, the nucleotide sequence encoding the IL-12 p35 subunit comprises the nucleotide sequence set forth in SEQ ID NO: 65.

In a specific embodiment, the sequence of the packaged genome comprising a transgene encoding IL-12 comprises the nucleotide sequence set forth in SEQ ID NO: 51.

In a specific embodiment, the sequence of the packaged genome comprising a transgene encoding IL-12 comprises the nucleotide sequence set forth in SEQ ID NO: 52.

In a specific embodiment, the sequence of the packaged genome comprising a transgene encoding IL-12 comprises the nucleotide sequence set forth in SEQ ID NO: 60.

In a specific embodiment, the chimeric NDV (or composition comprising the chimeric NDV) is administered to the subject via a route described in Section 5.5.1 and/or Section 6. In a specific embodiment, the chimeric NDV is administered to the subject intratumorally. In a specific embodiment, the intratumoral administration is subcutaneous. In another specific embodiment, the chimeric NDV is administered to the subject intra-nodally. In another embodiment, the chimeric NDV is administered to the subject intravenously.

In a specific embodiment, the antagonist of PD-1 or a ligand thereof (or composition comprising the antagonist) is administered to the subject via routes described in Section 5.5.1 and/or Section 6. In a specific embodiment, the antagonist of PD-1 or a ligand thereof (or composition comprising the antagonist) is administered to the subject intravenously.

In a specific embodiment, the cancer treated is a cancer described in Section 5.7.5 and/or Section 6. In a specific embodiment, the cancer is melanoma, kidney cancer, lung cancer, bladder cancer, or head and neck cancer. In a specific embodiment, the lung cancer is non-small cell lung cancer. In a specific embodiment, the head and neck cancer is squamous cell cancer of the head and neck. In a specific embodiment, the cancer is uterine cancer, gastric cancer, esophageal cancer, liver cancer, brain cancer or sarcoma. In a specific embodiment, the cancer is recurrent or relapsed. In a specific embodiment, the cancer is metastatic. In a specific embodiment, the cancer is unresectable. In a specific embodiment, the cancer comprises a dermal, subcutaneous, or nodal metastasis. In a specific embodiment, a biopsy of the cancer is PD-L1-positive as assessed by a technique known in the art or described herein, such as immunohistochemistry or the PD-L1 IHC 22C3 pharmDx (Agilent Technologies Inc.). In a specific embodiment, a biopsy is PD-L1-positive if the tumor proportion score (TPS), the percentage of cells staining for PD-L1 is at least 1%, 2%, 3%, 5%, 7%, 8%, 10%, 15%, 20%, 25%, 30%, 35%, 40%, 45%, 50%, 55%, 60%, 65%, 70%, 75%, 80%, 85%, 90%, 95%, 98%, or 100% as assessed by a technique known in the art or described herein such as immunohistochemistry or the PD-L1 IHC 22C3 pharmDx (Agilent Technologies Inc.). In another specific embodiment, a biopsy is PD-L1-positive if the tumor proportion score (TPS), the percentage of cells staining for PD-L1 is 1% to 100%, 25% to 50%, 25% to 100%, 50% to 75%, 50% to 100%, or 75% to 100%, as assessed by a technique known in the art or described herein such as immunohistochemistry or the PD-L1 IHC 22C3 pharmDx (Agilent Technologies Inc.). In other embodiments, a biopsy of the cancer is PD-L1-negative as assessed by a technique known in the art or described herein, such as immunohistochemistry or the PD-L1 IHC 22C3 pharmDx (Agilent Technologies Inc.). In a specific embodiment, a biopsy is PD-L1-negative if the tumor proportion score (TPS) is less than 1% as assessed by a technique known in the art or described herein, such as immunohistochemistry or the PD-L1 IHC 22C3 pharmDx (Agilent Technologies Inc.).

In a specific embodiment, the subject is a subject described in Section 5.7.3 and/or Section 6. In a specific embodiment, the subject is refractory to treatment with an antagonist of PD-1 or a ligand thereof. In a specific embodiment, the subject is refractory to treatment with nivolumab, AMP-224, MEDI0680, pembrolizumab, durvalumab, avelumab, bms-936559, or atezolizumab. In a specific embodiment, the subject is refractory to treatment with pembrolizumab alone. In a specific embodiment, the subject is unresponsive to treatment with pembrolizumab alone.

In a specific embodiment, the subject has relapsed cancer and is refractory to treatment with an antagonist of PD-1 or a ligand thereof. In a specific embodiment, the subject has relapsed cancer and is refractory to treatment with nivolumab, AMP-224, MEDI0680, pembrolizumab, durvalumab, avelumab, bms-936559, or atezolizumab. In a specific embodiment, the subject has relapsed cancer and is refractory to treatment with pembrolizumab.

### 5.7.2 Additional Methods

In one aspect, a chimeric NDV described herein (e.g., a chimeric NDV described in Section 5.2, supra) may be used in the treatment of cancer. In one embodiment, provided herein are methods for treating cancer, comprising administering to a subject in need thereof a chimeric NDV described herein (e.g., a chimeric NDV described in Section 5.2, supra) or a composition thereof. In a specific embodiment, provided herein is a method for treating cancer, comprising administering to a subject in need thereof an effective amount of a chimeric NDV described herein (e.g., a chimeric NDV described in Section 5.2, supra) or a composition thereof.

In a specific embodiment, the chimeric NDV used in the treatment of cancer comprises a packaged genome which encodes IL-12 (see, e.g., Section 5.2 and/or Section 6).

A chimeric NDV (e.g., a chimeric NDV described in Section 5.2, supra) described herein or a composition thereof, an oncolysate vaccine, or a whole cell cancer vaccine used in a method for treating cancer may be used as any line of therapy (e.g., a first, second, third, fourth or fifth line therapy).

In certain embodiments, a chimeric NDV described herein (e.g., a chimeric NDV described in Section 5.2, supra) is the only active ingredient administered to treat cancer. In specific embodiments, a chimeric NDV described herein (e.g., a chimeric NDV described in Section 5.2, supra) is the only active ingredient in a composition administered to treat cancer.

The chimeric NDV (e.g., a chimeric NDV described in Section 5.2, supra) or a composition thereof may be administered locally or systemically to a subject. For example, the chimeric NDV (e.g., a chimeric NDV described in Section 5.2, supra) or a composition thereof may be administered parenterally (e.g., intravenously, intraarterially, or subcutaneously), intratumorally, intra-nodally, intrapleurally, intranasally, intraperitoneally, intracavitary, intracranially, orally, rectally, by inhalation, intramuscularly, topically or intradermally to a subject. In a specific embodiment, the chimeric NDV is administered via the hepatic artery, by, e.g., hepatic artery injection, which can be performed by interventional radiology or through placement of an arterial infusion pump. In another specific embodiment, the chimeric NDV is administered intraoperatively, laparoscopically, endoscopically, or by image-guidance. In a specific embodiment, intraperitoneal administration of the chimeric NDV is performed by direct injection, infusion via catheter, or injection during laparoscopy. In a specific embodiment, the chimeric NDV is administered intratumorally. In certain embodiments, image-guidance is used to administer the chimeric NDV. In a specific embodiment, the chimeric NDV is administered intravenously. In another specific embodiment, the chimeric NDV is administered intra-nodally.

In certain embodiments, the methods described herein include the treatment of cancer for which no treatment is available. In some embodiments, a chimeric NDV described herein (e.g., a chimeric NDV described in Section 5.2, supra) or a composition thereof is administered to a subject to treat cancer as an alternative to other conventional therapies.

In one embodiment, provided herein is a method for treating cancer, comprising administering to a subject in need thereof a chimeric NDV described herein (e.g., a chimeric NDV described in Section 5.2, supra) or a composition thereof and one or more additional therapies, such as described in Section 5.7.6, infra. In a particular embodiment, one or more therapies are administered to a subject in combination with a chimeric NDV described herein (e.g., a chimeric NDV described in Section 5.2, supra) or a composition thereof to treat cancer. In a specific embodiment, the additional therapies are currently being used, have been used or are known to be useful in treating cancer. In another embodiment, a chimeric NDV described herein (e.g., a chimeric NDV described in Section 5.2, supra) or a composition thereof is administered to a subject in combination with a supportive therapy, a pain relief therapy, or other therapy that does not have a therapeutic effect on cancer. In a specific embodiment, the one or more additional therapies administered in combination with a chimeric NDV described herein (e.g., a chimeric NDV described in Section 5.2, supra) is one or more of the therapies described in Section 5.7.6.1, infra. In certain embodiments, a chimeric NDV described herein (e.g., a chimeric NDV described in Section 5.2, supra) and one or more additional therapies are administered in the same composition. In other embodiments, a chimeric NDV and one or more additional therapies are administered in different compositions.

In certain embodiments, two, three or multiple NDVs (including one, two or more chimeric NDVs described herein, such as one, two or more of the chimeric NDVs described in Section 5.2, supra) are administered to a subject to treat cancer. The second or more chimeric NDVs used in accordance with methods described herein that comprise administration of two, three or multiple NDVs to a subject to treat cancer may be naturally occurring chimeric NDVs or engineered chimeric NDVs that have been engineered to express heterologous amino acid sequence (e.g., a cytokine). The first and second chimeric NDVs may be part of the same pharmaceutical composition or different pharmaceutical compositions. In certain embodiments, the first chimeric NDV and the second chimeric NDV are administered by the same route of administration (e.g., both are administered intratumorally or intravenously). In other embodiments, the first chimeric NDV and the second chimeric NDV are administered by different routes of administration (e.g., one is administered intratumorally and the other is administered intravenously).

In another aspect, an NDV described herein (e.g., an NDV described in Section 5.1, supra) may be used in combination with one or more additional therapies, such as described herein in Section 5.7.6, infra (e.g., Section 5.7.6.1, infra), in the treatment of cancer. In one embodiment, provided herein are methods for treating cancer, comprising administering to a subject in need thereof an NDV described herein (e.g., an NDV described in Section 5.1, supra) or a composition thereof and one or more additional therapies, such as described herein in Section 5.7.6, infra. (e.g., Section 5.7.6.1). In a specific embodiment, provided herein is a method for treating cancer, comprising administering to a subject in need thereof an effective amount of an NDV described herein (e.g., an NDV described in Section 5.1, supra) or a composition thereof and an effective amount of one or more additional therapies, such as described in Section 5.7.6, infra. (e.g., Section 5.7.6.1). In certain embodiments, an NDV described herein (e.g., an NDV described in Section 5.1, supra) and one or more additional therapies, such as described in Section 5.7.6, infra (e.g., Section 5.7.6.1), are administered in the same composition. In other embodiments, an NDV (e.g., an NDV described in Section 5.1, supra) and one or more additional therapies are administered in different compositions.

The NDV used in combination with one or more additional therapies can be administered systemically or locally. For example, the NDV or composition thereof may be administered parenterally (e.g., intravenously, intraarterially, or subcutaneously), intratumorally, intra-nodally, intrapleurally, intranasally, intraperitoneally, intracranially, orally, rectally, by inhalation, intramuscularly, topically or intradermally to a subject. In a specific embodiment, the NDV is administered via the hepatic artery, by, e.g., hepatic artery injection, which can be performed by interventional radiology or through placement of an arterial infusion pump. In another specific embodiment, the NDV is administered intraoperatively, laparoscopically, or endoscopically. In a specific embodiment, intraperitoneal administration of the NDV is performed by direct injection, infusion via catheter, or injection during laparoscopy.

An NDV (e.g., an NDV described in Section 5.1, supra) described herein or a composition thereof, an oncolysate vaccine, or a whole cell cancer vaccine in combination with one or more additional therapies, such as described herein in Section 5.7.6, infra, may be used as any line of therapy (e.g., a first, second, third, fourth or fifth line therapy) for treating cancer in accordance with a method described herein.

In another aspect, whole cancer cells infected with a chimeric NDV described herein (e.g., a chimeric NDV described in Section 5.2, supra) can be used to treat cancer. In a specific embodiment, a chimeric NDV described herein (e.g., a chimeric NDV described in Section 5.2, supra) may be contacted with a cancer cell or a population of cancer cells and the infected cancer cell or population of cancer cells may be administered to a subject to treat cancer. In one embodiment, the cancer cells are subjected to gamma radiation prior to infection with a chimeric NDV described herein (e.g., a chimeric NDV described in Section 5.2, supra). In another embodiment, the cancer cells are subjected to gamma radiation after infection with a chimeric NDV described herein (e.g., a chimeric NDV described in Section 5.2, supra). In a particular embodiment, the cancer cells are treated prior to administration to a subject so that the cancer cells cannot multiply in the subject. In a specific embodiment, the cancer cells cannot multiply in the subject and the virus cannot infect the subject. In one embodiment, the cancer cells are subjected to gamma radiation prior to administration to subject. In another embodiment, the cancer cells are sonicated prior to administration to a subject. In another embodiment, the cancer cells are treated with mitomycin C prior to administration to a subject. In another embodiment, the cancer cells are treated by freezing and thawing prior to administration to a subject. In another embodiment, the cancer cells are treated with heat treatment prior to administration to a subject. The cancer cells may be administered locally or systemically to a subject. For example, the cancer cells may be administered parenterally (e.g., intravenously or subcutaneously), intratumorally, intra-nodally, intranasally, orally, by inhalation, intrapleurally, topically or intradermally to a subject. In a specific embodiment, the cancer cells are administered intratumorally or to the skin (e.g., intradermally) of a subject. The cancer cells used may be autologous or allogeneic. In a specific embodiment, the backbone of the chimeric NDV is a non-lytic strain. The cancer cells may be administered to a subject alone or in combination with an additional therapy. The cancer cells are preferably in a pharmaceutical composition. In certain embodiments, the cancer cells are administered in combination with one or more additional therapies, such as described in Section 5.7.6, infra. In certain embodiments, the cancer cells and one or more additional therapies are administered in the same composition. In other embodiments, the cancer cells and one or more additional therapies are administered in different compositions.

In another aspect, whole cancer cells infected with an NDV described herein (e.g., an NDV described in Section 5.1, supra) may be used in combination with one or more additional therapies described herein in Section 5.7.6, infra, in the treatment of cancer. In one embodiment, provided herein are methods for treating cancer, comprising administering to a subject in need thereof whole cancer cells infected with an NDV described herein (e.g., an NDV described in Section 5.1, supra) in combination with one or more additional therapies described herein in Section 5.7.6, infra. In a specific embodiment, provided herein is a method for treating cancer, comprising administering to a subject in need thereof an effective amount of whole cancer cells infected with an NDV described herein (e.g., an NDV described in Section 5.1, supra) in combination with an effective amount of one or more additional therapies described in Section 5.7.6, infra. In certain embodiments, whole cancer cells infected with an NDV described herein (e.g., an NDV described in Section 5.1, supra) and one or more additional therapies described in Section 5.7.6.1, infra, are administered in the same composition. In other embodiments, whole cancer cells infected with an NDV described herein (e.g., an NDV described in Section 5.1, supra) and one or more additional therapies are administered in different compositions.

In another aspect, a protein concentrate or plasma membrane preparation from lysed cancer cells infected with a chimeric NDV (e.g., a chimeric NDV described in Section 5.2, supra) can be used to treat cancer. In one embodiment, a plasma membrane preparation comprising fragments from cancer cells infected with a chimeric NDV described herein can be used to treat cancer. In another embodiment, a protein concentrate from cancer cells infected with a chimeric NDV described herein can be used to treat cancer. Techniques known to one of skill in the art may be used to produce the protein concentrate or plasma membrane preparation. In a specific embodiment, a chimeric NDV described herein (e.g., a chimeric NDV described in Section 5.2, supra) may be contacted with a cancer cell or a population of cancer cells and the infected cancer cell or population of cancer cells may be lysed using techniques known to one of skill in the art to obtain protein concentrate or plasma membrane fragments of the NDV-infected cancer cells, and the protein concentrate or plasma membrane fragments of the NDV-infected cancer cells may be administered to a subject to treat cancer. The protein concentrate or plasma membrane fragments may be administered locally or systemically to a subject. For example, the protein concentrate or plasma membrane fragments may be administered parenterally, intratumorally, intra-nodally, intranasally, intrapleurally, orally, by inhalation, topically or intradermally to a subject. In a specific embodiment, such a protein concentrate or plasma membrane preparation is administered intratumorally or to the skin (e.g., intradermally) of a subject. The cancer cells used to produce the protein concentrate or plasma membrane preparation may be autologous or allogeneic. In a specific embodiment, the backbone of the chimeric NDV is a lytic strain. The protein concentrate or plasma membrane preparation may be administered to a subject alone or in combination with an additional therapy. The protein concentrate or plasma membrane preparation is preferably in a pharmaceutical composition. In certain embodiments, the protein concentrate or plasma membrane preparation is administered in combination with one or more additional therapies, such as described in Section 5.7.6, infra (e.g., Section 5.7.6.1) In certain embodiments, the protein concentrate or plasma membrane preparation and one or more additional therapies are administered in the same composition. In other embodiments, the protein concentrate or plasma membrane preparation and one or more additional therapies are administered in different compositions.

In another aspect, a protein concentrate or plasma membrane preparation from lysed cancer cells infected with an NDV (e.g., an NDV described in Section 5.1, supra) may be used in combination with one or more additional therapies, such as described herein in Section 5.7.6, infra (e.g., Section 5.7.6.1), in the treatment of cancer. In one embodiment, provided herein are methods for treating cancer, comprising administering to a subject in need thereof a protein concentrate or plasma membrane preparation from lysed cancer cells infected with an NDV (e.g., an NDV described in Section 5.1, supra) in combination with one or more additional therapies, such as described herein in Section 5.7.6, infra. (e.g., Section 5.7.6.1). In a specific embodiment, provided herein is a method for treating cancer, comprising administering to a subject in need thereof an effective amount of a protein concentrate or plasma membrane preparation from lysed cancer cells infected with an NDV (e.g., an NDV described in Section 5.1, supra) in combination with an effective amount of one or more additional therapies, such as described in Section 5.7.6, infra. (e.g., Section 5.7.6.1). In certain embodiments, the protein concentrate or plasma membrane preparation and one or more additional therapies, such as described in Section 5.7.6, infra, are administered in the same composition. In other embodiments, the protein concentrate or plasma membrane preparation and one or more additional therapies are administered in different compositions.

In certain embodiments, the methods for treating cancer include those described in Section 5.6 of International Patent Application Publication No. WO 2014/158811 and U.S. Patent Application Publication Nos. 2016/0015760 A1 and 2014/0271677 A1, each of which is incorporated by reference herein in its entirety.

### 5.7.3 Patient Population

In some embodiments, an NDV (e.g., a chimeric NDV) described herein or a composition thereof, an oncolysate vaccine described herein, or a whole cell vaccine described herein, or a combination therapy described herein is administered to a subject suffering from cancer. In other embodiments, an NDV (e.g., a chimeric NDV) described herein or a composition thereof, an oncolysate vaccine described herein, or a whole cell vaccine described herein, or a combination therapy described herein is administered to a subject predisposed or susceptible to cancer. In some embodiments, an NDV (e.g., a chimeric NDV) or a composition thereof, an oncolysate vaccine described herein, or a whole cell vaccine described herein, or a combination therapy described herein is administered to a subject diagnosed with cancer. Specific examples of the types of cancer are described herein (see, e.g., Section 5.7.5 and Section 6). In an embodiment, the subject has metastatic cancer. In another embodiment, the subject has stage 1, stage 2, stage 3, or stage 4 cancer. In another embodiment, the subject is in remission. In yet another embodiment, the subject has a recurrence of cancer.

In certain embodiments, an NDV (e.g., a chimeric NDV) or a composition thereof, an oncolysate vaccine described herein, or a whole cell vaccine described herein, or a combination therapy described herein is administered to a human that is 0 to 6 months old, 6 to 12 months old, 6 to 18 months old, 18 to 36 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, an NDV (e.g., a chimeric NDV) or a composition thereof, an oncolysate vaccine described herein, or a whole cell vaccine described herein, or a combination therapy described herein is administered to a pediatric patient that is, e.g., 1 years old, 2 years old, 3 years, 4 years old, 5 years old, 6 years old, 7 years old, 8 years old, 9 years old, 10 years old, 11 years old, 12 years old, 13 years old, 14 years old, 15 years old, 16 years or 17 years old. In certain embodiments, an NDV (e.g., a chimeric NDV) or a composition thereof, an oncolysate vaccine described herein, or a whole cell vaccine described herein, or a combination therapy described herein is administered to a human pediatric patient that is 1 to 5 years old, 2 to 5 years old, 1 to 10 years old, 2 to 10 years old, 5 to 10 years old, 1 to 18 years old, 2 to 18 years old, 5 to 18 years old, or 10 to 18 years old. In some embodiments, an NDV (e.g., a chimeric NDV) or a composition thereof, an oncolysate vaccine described herein, or a whole cell vaccine described herein, or a combination therapy described herein is administered to a human infant. In other embodiments, an NDV (e.g., a chimeric NDV) or a composition thereof, an oncolysate vaccine described herein, or a whole cell vaccine described herein, or a combination therapy described herein is administered to a human toddler. In other embodiments, an NDV (e.g., a chimeric NDV) or a composition thereof, an oncolysate vaccine described herein, or a whole cell vaccine described herein, or a combination therapy described herein is administered to a human child. In some embodiments, an NDV (e.g., a chimeric NDV) or a composition thereof, an oncolysate vaccine described herein, or a whole cell vaccine described herein, or a combination therapy described herein is administered to an adult patient that is 18 years of age or older. In other embodiments, an NDV (e.g., a chimeric NDV) or a composition thereof, an oncolysate vaccine described herein, or a whole cell vaccine described herein, or a combination therapy described herein is administered to a human adult. In yet other embodiments, an NDV (e.g., a chimeric NDV) or a composition thereof, an oncolysate vaccine described herein, or a whole cell vaccine described herein, or a combination therapy described herein is administered to an elderly human. In a specific embodiment, an NDV (e.g., a chimeric NDV) or a composition thereof, an oncolysate vaccine described herein, or a whole cell vaccine described herein, or a combination therapy described herein is administered to a patient exhibits cutaneous or subcutaneous tumors or tumors within the lymp node.

In certain embodiments, an NDV (e.g., a chimeric NDV) or a composition thereof, an oncolysate vaccine described herein, or a whole cell vaccine described herein, or a combination therapy described herein is administered to a subject in an immunocompromised state or immunosuppressed state or at risk for becoming immunocompromised or immunosuppressed. In certain embodiments, an NDV (e.g., a chimeric NDV) or a composition thereof, an oncolysate vaccine described herein, or a whole cell vaccine described herein, or a combination therapy described herein is administered to a subject receiving or recovering from immunosuppressive therapy. In certain embodiments, an NDV (e.g., a chimeric NDV) or a composition thereof, an oncolysate vaccine described herein, or a whole cell vaccine described herein, or a combination therapy described herein is administered to a subject that has or is at risk of getting cancer. In certain embodiments, the subject is, will or has undergone surgery, chemotherapy and/or radiation therapy. In certain embodiments, the patient has undergone surgery to remove the tumor or neoplasm. In specific embodiments, the patient is administered an NDV (e.g., a chimeric NDV) or a composition thereof, an oncolysate vaccine described herein, or a whole cell vaccine described herein, or a combination therapy described herein following surgery to remove a tumor or neoplasm. In other embodiment, the patient is administered an NDV (e.g., a chimeric NDV) or a composition thereof, an oncolysate vaccine described herein, or a whole cell vaccine described herein, or a combination therapy described herein prior to undergoing surgery to remove a tumor or neoplasm. In certain embodiments, an NDV (e.g., a chimeric NDV) or a composition thereof, an oncolysate vaccine described herein, or a whole cell vaccine described herein, or a combination therapy described herein is administered to a subject that has, will have or had a tissue transplant, organ transplant or transfusion.

In some embodiments, an NDV (e.g., a chimeric NDV) or a composition thereof, an oncolysate vaccine described herein, or a whole cell vaccine described herein, or a combination therapy described herein is administered to a patient who has proven refractory to therapies other than the chimeric NDV or composition thereof, oncolysate, whole cell vaccine, or a combination therapy but are no longer on these therapies. In a specific embodiment, an NDV (e.g., a chimeric NDV) or a composition thereof, an oncolysate vaccine described herein, or a whole cell vaccine described herein, or a combination therapy described herein is administered to a patient who has proven refractory to chemotherapy. In a specific embodiment, an NDV (e.g., a chimeric NDV) or a composition thereof, an oncolysate vaccine described herein, or a whole cell vaccine described herein, or a combination therapy described herein is administered to a patient who has proven refractory or unresponsive to treatment with an antagonist of PD-1 or a ligand thereof. In a specific embodiment, an NDV (e.g., a chimeric NDV) or a composition thereof, an oncolysate vaccine described herein, or a whole cell vaccine described herein, or a combination therapy described herein is administered to a patient who has proven refractory or unresponsive to monotherapy treatment with an antagonist of PD-1 or a ligand thereof. In a specific embodiment, an NDV (e.g., a chimeric NDV) or a composition thereof, an oncolysate vaccine described herein, or a whole cell vaccine described herein, or a combination therapy described herein is administered to a patient who has proven refractory or unresponsive to treatment with a PD-1-blocking antibody (e.g., pembrolizumab or nivolumab). In a specific embodiment, an NDV (e.g., a chimeric NDV) or a composition thereof, an oncolysate vaccine described herein, or a whole cell vaccine described herein, or a combination therapy described herein is administered to a patient who has proven refractory or unresponsive to monotherapy treatment with a PD-1-blocking antibody (e.g., pembrolizumab or nivolumab). In a specific embodiment, an NDV (e.g., a chimeric NDV) or a composition thereof, an oncolysate vaccine described herein, or a whole cell vaccine described herein, or a combination therapy described herein is administered to a patient who has proven refractory or unresponsive to treatment with a PD-L1-blocking antibody (e.g., atezolizumab). In another specific embodiment, an NDV (e.g., a chimeric NDV) or a composition thereof, an oncolysate vaccine described herein, or a whole cell vaccine described herein, or a combination therapy described herein is administered to a patient who has proven refractory or unresponsive to monotherapy treatment with a PD-L1-blocking antibody (e.g., atezolizumab). In a specific embodiment, the therapy to which the patient has proven refractory is part of the combination therapy. For example, in a specific embodiment, the patient has proven refractory to treatment with an antagonist of PD-1 or a ligand thereof; however, without being bound to any particular theory, the patient is responsive to treatment with the antagonist of PD-1 or a ligand thereof in combination with an NDV (e.g., a chimeric NDV) or a composition thereof, an oncolysate vaccine described herein, or a whole cell vaccine described herein, or a combination therapy described herein. The determination of whether cancer is refractory can be made by any method known in the art. In a certain embodiment, refractory patient is a patient refractory to a standard therapy. In some embodiments, a patient with cancer is initially responsive to therapy, but subsequently becomes refractory.

In certain embodiments, the patient to be treated in accordance with the methods described herein is a patient that has relapsed after treatment with therapies other than the chimeric NDV or composition thereof, oncolysate, whole cell vaccine, or a combination therapy. In some embodiments, the patient to be treated in accordance with the methods described herein is a patient that has relapsed after treatment with an antagonist of PD-1 or a ligand thereof. In certain embodiments, the patient to be treated in accordance with the methods described herein is a patient that has relapsed after monotherapy treatment with an antagonist of PD-1 or a ligand thereof. In some embodiments, the patient to be treated in accordance with the methods described herein is a patient that has relapsed after treatment with a PD-1-blocking antibody (e.g., pembrolizumab or nivolumab). In certain embodiments, the patient to be treated in accordance with the methods described herein is a patient that has relapsed after monotherapy treatment with a PD-1-blocking antibody (e.g., pembrolizumab or nivolumab). In some embodiments, the patient to be treated in accordance with the methods described herein is a patient that has relapsed after treatment with a PD-L1-blocking antibody (e.g., atezolizumab). In certain embodiments, the patient to be treated in accordance with the methods described herein is a patient that has relapsed after monotherapy treatment with a PD-L1-blocking antibody (e.g., atezolizumab).

In certain embodiments, the patient to be treated in accordance with the methods described herein is a patient that has relapsed after treatment with therapies other than the chimeric NDV or composition thereof, oncolysate, whole cell vaccine, or a combination therapy and is refractory or unresponsive to treatment with an antagonist of PD-1 or a ligand thereof. In some embodiments, the patient to be treated in accordance with the methods described herein is a patient that has relapsed after treatment with an antagonist of PD-1 or a ligand thereof and is refractory or unresponsive to monotherapy treatment with an antagonist of PD-1 or a ligand thereof. In certain embodiments, the patient to be treated in accordance with the methods described herein is a patient that has relapsed after treatment with a PD-1-blocking antibody (e.g., pembrolizumab or nivolumab) and is refractory or unresponsive to monotherapy treatment with a PD-1 blocking antibody. In certain embodiments, the patient to be treated in accordance with the methods described herein is a patient that has relapsed after treatment with with a PD-L1-blocking antibody (e.g., atezolizumab) and is refractory or unresponsive to monotherapy treatment with a PD-L1-blocking antibody.

In certain embodiments, the patient to be treated in accordance with the methods described herein is a patient already being treated with antibiotics, anti-virals, anti-fungals, or other biological therapy/immunotherapy or anti-cancer therapy. Among these patients are refractory patients, and patients who are too young for conventional therapies. In some embodiments, the subject being administered an NDV (e.g., a chimeric NDV), an oncolysate vaccine described herein, or a whole cell vaccine described herein, or a combination therapy described herein has not received therapy prior to the administration of the chimeric NDV or composition, the oncolysate vaccine, or the whole cell vaccine, or the combination therapy.

In some embodiments, an NDV (e.g., a chimeric NDV) or a composition thereof, an oncolysate vaccine described herein, or a whole cell vaccine described herein, or a combination therapy described herein is administered to a patient to prevent the onset of cancer in a patient at risk of developing cancer. In some embodiments, compounds are administered to a patient who are susceptible to adverse reactions to conventional therapies.

In some embodiments, the subject being administered an NDV (e.g., a chimeric NDV) or a composition thereof, an oncolysate vaccine described herein, or a whole cell vaccine described herein, or a combination therapy described herein has not received prior therapy. In other embodiments, an NDV (e.g., a chimeric NDV) or a composition thereof, an oncolysate vaccine described herein, or a whole cell vaccine described herein, or a combination therapy described herein is administered to a subject who has received a therapy prior to administration of the NDV (e.g., a chimeric NDV) or composition, the oncolysate vaccine, the whole cell vaccine, or the combination therapy. In some embodiments, the subject administered an NDV (e.g., a chimeric NDV) or a composition thereof, an oncolysate vaccine described herein, or a whole cell vaccine described herein, or a combination therapy described herein experienced adverse side effects to a prior therapy or a prior therapy was discontinued due to unacceptable levels of toxicity to the subject.

In a specific embodiment, the subject being administered an NDV (e.g., a chimeric NDV) or a composition thereof, an oncolysate vaccine described herein, or a whole cell vaccine described herein, or a combination therapy described herein has one or more PD-L1 positive tumors or malignancies. In a specific embodiment, a tumor or malignancy is PD-L1-positive. In a specific embodiment, a tumor or malignancy is PD-L1-positive if the tumor proportion score (TPS) of a biopsy of the tumor or malignancy, the percentage of cells staining for PD-L1 is at least 1%, 2%, 3%, 5%, 7%, 8%, 10%, 15%, 20%, 25%, 30%, 35%, 40%, 45%, 50%, 55%, 60%, 65%, 70%, 75%, 80%, 85%, 90%, 95%, 98%, or 100% as assessed by a technique known in the art or described herein such as immunohistochemistry or the PD-L1 IHC 22C3 pharmDx (Agilent Technologies Inc.). In another specific embodiment, a tumor or malignancy is PD-L1-positive if the tumor proportion score (TPS) of a biopsy of the tumor or malignancy, the percentage of cells staining for PD-L1 is 1% to 100%, 25% to 50%, 25% to 100%, 50% to 75%, 50% to 100%, or 75% to 100%, as assessed by a technique known in the art or described herein such as immunohistochemistry or the PD-L1 IHC 22C3 pharmDx (Agilent Technologies Inc.).

In a specific embodiment, a tumor or malignancy is PD-L1-positive if the combined positive score (CPS) of a biopsy of the tumor or malignancy, the percentage of cells staining for PD-L1 is at least 1%, 2%, 3%, 5%, 7%, 8%, 10%, 15%, 20%, 25%, 30%, 35%, 40%, 45%, 50%, 55%, 60%, 65%, 70%, 75%, 80%, 85%, 90%, 95%, 98%, or 100% as assessed by a technique known in the art or described herein such as immunohistochemistry or the PD-L1 IHC 22C3 pharmDx (Agilent Technologies Inc.). In another specific embodiment, a tumor or malignancy is PD-L1-positive if the combined positive score (CPS) of a biopsy of the tumor or malignancy, the percentage of cells staining for PD-L1 is 1% to 100%, 25% to 50%, 25% to 100%, 50% to 75%, 50% to 100%, or 75% to 100%, as assessed by a technique known in the art or described herein such as immunohistochemistry or the PD-L1 IHC 22C3 pharmDx (Agilent Technologies Inc.). In order to determine the CPS, the number of viable PD-L1 positive tumor cells, the number of viable PD-L1 negative tumor cells, and the number of viable PD-L1 positive mononuclear inflammatory cells (MIC) in a tumor tissue sample from a subject is determined, and then the formula is used to calculate the combined positive score (CPS): CPS=(number of PD-L1 positive tumor cells+number of PD-L1 positive mononuclear inflammatory cells (MIC)/number of PD-L-1 positive tumor cells+PD-L1 negative tumor cells)×100%. See, e.g., U.S. Patent Application Publication No. 2017/0285037 and Kulangara et al., Journal of Clinical Oncology 2017 35: 15_suppl, e14589-e14589 for a description of the combined positive score (CPS), each of which is incorporated herein by reference in its entirety.

In some embodiments, the subject being administered an NDV (e.g., a chimeric NDV) or a composition thereof, an oncolysate vaccine described herein, or a whole cell vaccine described herein, or a combination therapy described herein has one or more PD-L1 negative tumors or malignancies. In a specific embodiment, a tumor or malignancy is PD-L1 negative. In a specific embodiment, a biopsy of the cancer is PD-L1-negative as assessed by a technique known in the art or described herein, such as immunohistochemistry or the PD-L1 IHC 22C3 pharmDx (Agilent Technologies Inc.). In a specific embodiment, a tumor or malignancy is PD-L1-negative if the tumor proportion score (TPS) of a biopsy of the tumor or malignancy, the percentage of cells staining for PD-L1 is less than 1% as assessed by a technique known in the art or described herein, such as immunohistochemistry or the PD-L1 IHC 22C3 pharmDx (Agilent Technologies Inc.).

In a specific embodiment, a tumor or malignancy is PD-L1-negative if the combined positive score (CPS) of a biopsy of the tumor or malignancy, the percentage of cells staining for PD-L1 is less than 1% as assessed by a technique known in the art or described herein, such as immunohistochemistry or the PD-L1 IHC 22C3 pharmDx (Agilent Technologies Inc.).

In a specific embodiment, the subject being administered an NDV (e.g., a chimeric NDV) or a composition thereof, an oncolysate vaccine described herein, or a whole cell vaccine described herein, or a combination therapy described herein has one or more tumors or malignancies that has low levels of PD-L1. In a specific embodiment, a tumor or malignancy has low levels of PD-L1 expression if a biopsy of the tumor or malignancy has the tumor proportion score (TPS) of between 1% to 50%, or 1% to 40%, 1% to 30%, 1% to 25%, 1% to 15%, or 1% to 10% as assessed by a technique known in the art or described herein, such as immunohistochemistry or the PD-L1 IHC 22C3 pharmDx (Agilent Technologies Inc.). In a specific embodiment, a tumor or malignancy has low levels of PD-L1 expression if a biopsy of the tumor or malignancy has the tumor proportion score (TPS) of less than 50% but 1% or greater as assessed by a technique known in the art or described herein, such as immunohistochemistry or the PD-L1 IHC 22C3 pharm Dx (Agilent Technologies Inc.)

In a specific embodiment, a tumor or malignancy has low levels of PD-L1 expression if a biopsy of the tumor or malignancy has the combined positive score (CPS) of between 1% to 50%, or 1% to 40%, 1% to 30%, 1% to 25%, 1% to 15%, or 1% to 10% as assessed by a technique known in the art or described herein, such as immunohistochemistry or the PD-L1 IHC 22C3 pharmDx (Agilent Technologies Inc.). In a specific embodiment, a tumor or malignancy has low levels of PD-L1 expression if a biopsy of the tumor or malignancy has the combined positive score (CPS) of less than 50% but 1% or greater as assessed by a technique known in the art or described herein, such as immunohistochemistry or the PD-L1 IHC 22C3 pharmDx (Agilent Technologies Inc.)

In a specific embodiment, an ELISA is used to determine if tumor cells are PD-L1-positive and/or to determine if a tumor or malignancy has low levels of PD-L1. In a specific embodiment, immunohistochemistry is used to determine if tumor cells are PD-L1-negative, PD-L1-positive and/or if a tumor or malignancy has low levels of PD-L1. In a specific embodiment, a tumor or malignancy (or a biopsy thereof) is determined to be PD-L1-negative, PD-L1-positive, and/or have low levels of PD-L1 according to one or more assays approved by the U.S. Food and Drug Administration for determining the level of PD-L1. Nonlimiting examples of U.S. Food and Drug Administration-approved assays for determining the level of PD-L1 include PD-L1 IHC 22C3 pharmDx (manufactured by Dako North America, Inc.) and Ventana PD-L1 (SP142) Assay (manufactured by Ventana Medical Systems, Inc.). In another specific embodiment, the level of PD-L1 in a tumor or malignancy (or a biopsy thereof) is determined according to a laboratory-developed test performed in a Clinical Laboratory Improvement Amendments-certified laboratory.

### 5.7.4 Dosage & Frequency

The amount of an NDV or a composition thereof, an oncolysate vaccine, or a whole cell vaccine which will be effective in the treatment of cancer will depend on the nature of the cancer, the route of administration, the general health of the subject, etc. and should be decided according to the judgment of a medical practitioner. Standard clinical techniques, such as in vitro assays, may optionally be employed to help identify optimal dosage ranges. However, suitable dosage ranges of an NDV for administration are generally about 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and most preferably about 10⁴ to about 10¹², 10⁶ to 10¹², 10⁸ to 10¹², 10⁹ to 10¹² or 10⁹ to 10¹¹ pfu, and can be administered to a subject once, twice, three, four or more times with intervals as often as needed. Dosage ranges of oncolysate vaccines for administration may include 0.001 mg, 0.005 mg, 0.01 mg, 0.05 mg. 0.1 mg. 0.5 mg, 1.0 mg, 2.0 mg. 3.0 mg, 4.0 mg, 5.0 mg, 10.0 mg, 0.001 mg to 10.0 mg, 0.01 mg to 1.0 mg, 0.1 mg to 1 mg, and 0.1 mg to 5.0 mg, and can be administered to a subject once, twice, three or more times with intervals as often as needed. Dosage ranges of whole cell vaccines for administration may include 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² cells, and can be administered to a subject once, twice, three or more times with intervals as often as needed. In certain embodiments, dosages similar to those currently being used in clinical trials for NDV, oncolysate vaccines or whole cell vaccines are administered to a subject. Effective doses may be extrapolated from dose response curves derived from in vitro or animal model test systems.

In certain embodiments, an NDV (e.g., a chimeric NDV) or a composition thereof is administered to a subject as a single dose followed by a second dose 1 to 6 weeks, 1 to 5 weeks, 1 to 4 weeks, 1 to 3 weeks, 1 to 2 weeks later. In accordance with these embodiments, booster inoculations may be administered to the subject at 6 to 12 month intervals following the second inoculation. In certain embodiments, an oncolysate vaccine or a whole cell vaccine is administered to a subject as a single dose followed by a second dose 1 to 6 weeks, 1 to 5 weeks, 1 to 4 weeks, 1 to 3 weeks, 1 to 2 weeks later.

In certain embodiments, administration of the same NDV (e.g., chimeric NDV) or a composition thereof, oncolysate vaccine, or whole cell vaccine may be repeated and the administrations may be separated by at least 1 day, 2 days, 3 days, 5 days, 6 says, 7 days, 10 days, 14 days, 15 days, 21 days, 28 days, 30 days, 45 days, 2 months, 75 days, 3 months, or at least 6 months. In other embodiments, administration of the same NDV (e.g., a NDV) or a composition thereof, oncolysate vaccine, or whole cell vaccine may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first NDV (e.g., a first chimeric NDV) or a composition thereof is administered to a subject followed by the administration of a second NDV (e.g., a second chimeric NDV) or a composition thereof. In certain embodiments, the first and second NDVs (e.g., the first and second chimeric NDVs) or compositions thereof may be separated by at least 1 day, 2 days, 3 days, 5 days, 6 days, 7 days, 10 days, 14 days, 15 days, 21 days, 28 days, 30 days, 45 days, 2 months, 75 days, 3 months, or at least 6 months. In other embodiments, the first and second NDVs (e.g., the first and second chimeric NDVs) or compositions thereof may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months.

In certain embodiments, an NDV or composition thereof, or oncolysate vaccine or whole cell vaccine is administered to a subject in combination with one or more additional therapies, such as a therapy described in Section 5.7.6, infra. The dosage of the other one or more additional therapies will depend upon various factors including, e.g., the therapy, the nature of the cancer, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. In other embodiments, the dose of the other therapy is a lower dose and/or less frequent administration of the therapy than recommended for the therapy for use as a single agent is used in accordance with the methods disclosed herein. Recommended doses for approved therapies can be found in the Physician's Desk Reference.

In certain embodiments, an NDV or composition thereof, or oncolysate vaccine or whole cell vaccine is administered to a subject concurrently with the administration of one or more additional therapies. In other embodiments, an NDV or composition thereof, or oncolysate vaccine or whole cell vaccine is administered to a subject every 3 to 7 days, 1 to 6 weeks, 1 to 5 weeks, 1 to 4 weeks, 2 to 4 weeks, 1 to 3 weeks, or 1 to 2 weeks and one or more additional therapies (such as described in Section 5.7.6, infra) is administered every 3 to 7 days, 1 to 6 weeks, 1 to 5 weeks, 1 to 4 weeks, 1 to 3 weeks, or 1 to 2 weeks. In certain embodiments, an NDV or composition thereof, or oncolysate vaccine or whole cell vaccine is administered to a subject every 1 to 2 weeks and one or more additional therapies (such as described in Section 5.7.6, infra) is administered every 2 to 4 weeks. In some embodiments, an NDV or composition thereof, or oncolysate vaccine or whole cell vaccine is administered to a subject every week and one or more additional therapies (such as described in Section 5.7.6, infra) is administered every 2 weeks.

The dosage of the antagonist of PD-1 or a ligand thereof used to treat a subject will depend upon various factors including, e.g., the therapy, the nature of the cancer, the route of administration, the general health of the subject, etc. and should be decided according to the judgment of a medical practitioner. In specific embodiments, the dose of the antagonist of PD-1 or a ligand thereof is the dose and/or frequency of administration of the antagonist of PD-1 or a ligand thereof recommended for the antagonist of PD-1 or a ligand thereof for use as a single agent is used in accordance with the methods disclosed herein. In some embodiments, the dose of the antagonist of PD-1 or a ligand thereof is a lower dose and/or less frequent administration of the therapy than recommended for the antagonist of PD-1 or a ligand thereof for use as a single agent is used in accordance with the methods disclosed herein. Recommended doses for approved therapies can be found in the Physician's Desk Reference.

In a specific embodiment in which the antagonist of PD-1 or a ligand thereof is nivolumab, the dosage of nivolumab may be 240 mg as an intravenous infusion over a period of time, e.g., 30 minutes, every two weeks. See, e.g., Full Prescribing Information for OPDIVO, as revised April 2018, which is incorporated by reference herein in its entirety. In another specific embodiment in which the antagonist of PD-1 or a ligand thereof is nivolumab, the dosage of nivolumab may be 480 mg as an intravenous infusion over a period of time, e.g., 30 minutes, every four weeks. Id. In another specific embodiment in which the antagonist of PD-1 or a ligand thereof is nivolumab, the dosage of nivolumab may be 3 mg/kg as an intravenous infusion over a period of time, e.g., 60 minutes, every two weeks. See, e.g., Full Prescribing Information for OPDIVO, Reference ID: 3677021, which is incorporated by reference herein in its entirety. In a specific embodiment in which the antagonist of PD-1 or a ligand thereof is pembrolizumab, the dosage of pembrolizumab may be 200 mg as an intravenous infusion over a period of time, e.g., 30 minutes, every three weeks. See, e.g., Full Prescribing Information for KEYTRUDA, as revised November 2017, which is incorporated by reference herein in its entirety. In another specific embodiment in which the antagonist of PD-1 or a ligand thereof is pembrolizumab, the dosage of pembrolizumab may be 2 mg/kg as an intravenous infusion over a period of time, e.g., 30 minutes, every three weeks. See, e.g., Full Prescribing Information for KEYTRUDA, Reference ID: 3862712, which is incorporated by reference herein in its entirety. In another specific embodiment in which the antagonist of PD-1 or a ligand thereof is pembrolizumab, the dosage of pembrolizumab may be between 2 mg/kg and up to 200 mg/kg as an intravenous infusion over a period of time, e.g., 30 minutes, every three weeks. See, e.g., Full Prescribing Information for KEYTRUDA, Reference ID: 3862712, which is incorporated by reference herein in its entirety. In a specific embodiment in which the antagonist of PD-1 or a ligand thereof is atezolizumab, the dosage of atezolizumab may be 1,200 mg as an intravenous infusion over a period of time, e.g., 60 minutes, every three weeks. See, e.g., Full Prescribing Information for TECENTRIQ, Reference ID: 4000525, which is incorporated by reference herein in its entirety.

### 5.7.5 Types of Cancer

Specific examples of cancers that can be treated in accordance with the methods described herein include, but are not limited to: leukemias, such as but not limited to, acute leukemia, acute lymphocytic leukemia, acute myelocytic leukemias, such as, myeloblastic, promyelocytic, myelomonocytic, monocytic, and erythroid leukemias and myelodysplastic syndrome; myelofibrisis, chronic leukemias, such as but not limited to, chronic myelocytic (granulocytic) leukemia, chronic lymphocytic leukemia, hairy cell leukemia; polycythemia vera; lymphomas such as but not limited to Hodgkin disease, non-Hodgkin disease; multiple myelomas such as but not limited to smoldering multiple myeloma, nonsecretory myeloma, osteosclerotic myeloma, placancer cell leukemia, solitary placancercytoma and extramedullary placancercytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, glioblastoma multiforme, acoustic neurinoma, craniopharyngioma, medulloblastoma, meningioma, pineocytoma, pineoblastoma, primary brain lymphoma; breast cancer including but not limited to triple negative breast cancer, ER+/HER2-breast cancer, ductal carcinoma, adenocarcinoma, lobular (cancer cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, placancercytoma, verrucous carcinoma, and oat cell (cancer cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and cancer-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In a specific embodiment, the chimeric NDVs described herein or compositions thereof, an oncolysate vaccine described herein, a whole cell vaccine herein, or a combination therapy described herein are useful in the treatment of a variety of cancers and abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma.

In some embodiments, cancers associated with aberrations in apoptosis are treated in accordance with the methods described herein. Such cancers may include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus are treated in accordance with the methods described herein. In other specific embodiments, a sarcoma or melanoma is treated in accordance with the methods described herein.

In a specific embodiment, the cancer being treated in accordance with the methods described herein is leukemia, lymphoma or myeloma (e.g., multiple myeloma). Specific examples of leukemias and other blood-borne cancers that can be treated in accordance with the methods described herein include, but are not limited to, acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, and hairy cell leukemia.

Specific examples of lymphomas that can be treated in accordance with the methods described herein include, but are not limited to, Hodgkin disease, non-Hodgkin lymphoma such as diffuse large B-cell lymphoma, multiple myeloma, Waldenström's macroglobulinemia, heavy chain disease, and polycythemia vera.

In another embodiment, the cancer being treated in accordance with the methods described herein is a solid tumor. Examples of solid tumors that can be treated in accordance with the methods described herein include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, cancer cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma. In another embodiment, the cancer being treated in accordance with the methods described herein is a metastatic. In another embodiment, the cancer being treated in accordance with the methods described herein is malignant.

In a specific embodiment, the cancer being treated in accordance with the methods described herein is melanoma (e.g. advanced melanoma), non-small cell lung cancer (NSCLC), head and neck squamous cell cancer (HNSCC), classical Hodgkin lymphoma, advanced urothelial cancer, a microsatellite instability-high cancer, or gastric or gastroesophageal junction adenocarcinoma. In a specific embodiment, the cancer being treated in accordance with the methods described herein is uterine cancer, gastric cancer, esophageal cancer, liver cancer, brain cancer, or sarcoma. In a specific embodiment, the cancer being treated in accordance with the methods described herein is (1) refractory classical Hodgkin lymphoma, (2) recurrent or metastatic head and neck squamous cell cancer, (3) unresectable or metastatic melanoma, (4) locally or advanced or metastatic urothelial carcinoma, (5) recurrent locally advanced or metastatic gastric or gastroesophageal adenocarcinoma with tumors expressing programmed death-ligand 1 (“PD-L1”) (e.g., tumors having a CPS≥1), (6) unresectable or metastatic, microsatellite instability-high cancer or mismatch repair deficient solid tumors that have progressed following prior treatment and who have no satisfactory alternative treatment options, or colorectal cancer that has progessed following treatment with a fluoropyrimidine, oxaliplatin and irinotecan, or (7) metastatic non-small cell lung cancers having tumors which express PD-L1 (e.g., tumors having a TPS≥1% or 50%).

In a specific embodiment, the cancer being treated in accordance with the methods described herein is melanoma, non-small cell lung cancer, head and neck cancer (HNSCC head and neck squamous cell carcinoma), Urothelial cancer, Triple negative breast cancer, gastric cancer, classical Hodgkin lymphoma, non-Hodgkin lymphoma, primary mediastinal B-cell lymphoma, mesothelioma, ovarian cancer, small cell lung cancer, esophageal cancer, nasopharyngeal cancer, anal cancer, biliary tract cancer, colorectal cancer, ER+/HER2-breast cancer, cervical cancer, thyroid cancer, salivary cancer, endometrial cancer, prostate cancer, glioblastoma, microsatellite instability-high (MSI-H) or mismatch repair deficient cancer (tissue agnostic), or tumors with high tumor mutational burden (tissue agnostic).

In a specific embodiment, the cancer being treated in accordance with the methods described herein is a cancer that has a poor prognosis and/or has a poor response to conventional therapies, such as chemotherapy and radiation. In another specific embodiment, the cancer being treated in accordance with the methods described herein is malignant melanoma, malignant glioma, renal cell carcinoma, pancreatic adenocarcinoma, malignant pleural mesothelioma, lung adenocarcinoma, lung small cell carcinoma, lung squamous cell carcinoma, anaplastic thyroid cancer, and head and neck squamous cell carcinoma. In another specific embodiment, the cancer being treated in accordance with the methods described herein is a type of cancer described in Section 6, infra.

In a specific embodiment, the cancer being treated in accordance with the methods described herein is refractory Hodgkin lymphoma, recurrent or metastatic head and neck squamous cell cancer, unresectable or metastatic melanoma, or metastatic non-small cell lung cancer.

In a specific embodiment, a cancer being treated in accordance with the methods described herein is PD-L1-positive. In a specific embodiment, a cancer is PD-L1-positive if the tumor proportion score (TPS), the percentage of cells staining for PD-L1 in a biopsy of the cancer is at least 1%, 2%, 3%, 5%, 7%, 8%, 10%, 15%, 20%, 25%, 30%, 35%, 40%, 45%, 50%, 55%, 60%, 65%, 70%, 75%, 80%, 85%, 90%, 95%, 98%, or 100% as assessed by a technique known in the art or described herein such as immunohistochemistry or the PD-L1 IHC 22C3 pharmDx (Agilent Technologies Inc.). In another specific embodiment, a cancer is PD-L1-positive if the TPS, the percentage of cells staining for PD-L1 in a biopsy of the cancer is at least 1% or 1% to 100%, as assessed by a technique known in the art or described herein such as immunohistochemistry or the PD-L1 IHC 22C3 pharmDx (Agilent Technologies Inc.). In another specific embodiment, a cancer is PD-L1-positive if the TPS, the percentage of cells staining for PD-L1 in a biopsy of the cancer is 1% to 100%, 25% to 50%, 25% to 100%, 50% to 75%, 50% to 100%, or 75% to 100%, as assessed by a technique known in the art or described herein such as immunohistochemistry or the PD-L1 IHC 22C3 pharmDx (Agilent Technologies Inc.). In a specific embodiment, the cancer that is determined to be PD-L1 positive using the TPS score is non-small cell lung cancer.

In a specific embodiment, a cancer is PD-L1-positive if the combined positive score (CPS), the percentage of cells staining for PD-L1 in a biopsy of the cancer is at least 1%, 2%, 3%, 5%, 7%, 8%, 10%, 15%, 20%, 25%, 30%, 35%, 40%, 45%, 50%, 55%, 60%, 65%, 70%, 75%, 80%, 85%, 90%, 95%, 98%, or 100% as assessed by a technique known in the art or described herein such as immunohistochemistry or the PD-1.1 IHC 22C3 pharmDx (Agilent Technologies Inc.). In another specific embodiment, a cancer is PD-L1-positive if the CPS, the percentage of cells staining for PD-L1 in a biopsy of the cancer is at least 1% or 1% to 100%, as assessed by a technique known in the art or described herein such as immunohistochemistry or the PD-L1 IHC 22C3 pharmDx (Agilent Technologies Inc.). In another specific embodiment, a cancer is PD-L1-positive if the CPS, the percentage of cells staining for PD-L1 in a biopsy of the cancer is 1% to 100%, 25% to 50%, 25% to 100%, 50% to 75%, 50% to 100%, or 75% to 100%, as assessed by a technique known in the art or described herein such as immunohistochemistry or the PD-L1 IHC 22C3 pharmDx (Agilent Technologies Inc.). In a specific embodiment, the cancer that is determined to be PD-L1 positive using the CPS score is gastric cancer (e.g., recurrent locally advanced metastatic gastric or gastroesophageal junction adenocarcinoma).

In some specific embodiments, a cancer being treated in accordance with the methods described herein is PD-L1-negative. In a specific embodiment, a cancer is PD-L1-negative if the TPS, the percentage of cells staining for PD-L1 in a biopsy of the cancer is less than 1% as assessed by a technique known in the art or described herein, such as immunohistochemistry or the PD-L1 IHC 22C3 pharmDx (Agilent Technologies Inc.). In a specific embodiment, the cancer that is determined to be PD-L1 negative using the TPS score is non-small cell lung cancer.

In specific embodiments, a cancer being treated in accordance with the methods described herein has low levels of PD-L1 expression. In a specific embodiment, a cancer has low levels of PD-L1 expression if the TPS, the percentage of cells staining for PD-L1 in a biopsy of the cancer is between 1% to 50%, or 1% to 40%, 1% to 30%, 1% to 25%, 1% to 15%, or 1% to 10% as assessed by a technique known in the art or described herein, such as immunohistochemistry or the PD-L1 IHC 22C3 pharmDx (Agilent Technologies Inc.). In a specific embodiment, a cancer has low levels of PD-L1 expression if the TPS, the percentage of cells staining for PDL1 in a biopsy of the cancer is less than 50% but 1% or greater as assessed by a technique known in the art or described herein, such as immunohistochemistry or the PD-L1 IHC 22C3 pharmDx (Agilent Technologies Inc.)

In a specific embodiment, ELISA is used to determine if a biopsy of cancer is PD-L1-positive, PD-L1-negative, and/or have low levels of PD-L1 expression. In a specific embodiment, immunohistochemistry is used to determine if a biopsy of cancer is PD-L1-positive, PD-L1-negative, and/or have low levels of PD-L1 expression. In a specific embodiment, a tumor or malignancy (or a biopsy thereof) is determined to be PD-L1-positive, PD-L1 negative, and/or have low levels of PD-L1 expression according to one or more assays approved by the U.S. Food and Drug Administration for determining the level of PD-L1. Non-limiting examples of U.S. Food and Drug Administration-approved assays for determining the level of PD-L1 include PD-L1 IHC 22C3 pharmDx (manufactured by Dako North America, Inc.) and Ventana PD-L1 (SP142) Assay (manufactured by Ventana Medical Systems, Inc.). In another specific embodiment, the level of PD-L1 in a tumor or malignancy (or a biopsy thereof) is determined according to a laboratory-developed test performed in a Clinical Laboratory Improvement Amendments-certified laboratory. In another specific embodiment, the level of PD-L1 in a tumor or malignancy (or a biopsy thereof) is determined using PCR. In another specific embodiment, the level of PD-L1 in a tumor or malignancy (or a biopsy thereof) is determined by assessing the gene expression profile of certain tumor-associated genes using kits from NanoString Technologies.

In specific embodiments, a cancer being treated in accordance with the methods described herein is refractory or unresponsive to monotherapy treatment with an antagonist of PD-1 or a ligand thereof. In a specific embodiment, a cancer being treated in accordance with the methods described herein is refractory or unresponsive to monotherapy treatment with an PD-1 blocking antibody (e.g., nivolumab or pembrolizumab). In specific embodiments, a cancer being treated in accordance with the methods described herein is refractory or unresponsive to monotherapy treatment with a PD-L1 blocking antibody (e.g., avelumab).

In specific embodiments, a cancer being treated in accordance with the methods described herein is PD-L1-negative and refractory or unresponsive to monotherapy treatment with an antagonist of PD-1 or a ligand thereof. In a specific embodiment, a cancer being treated in accordance with the methods described herein is PD-L1-negative and refractory or unresponsive to monotherapy treatment with a PD-1 blocking antibody (e.g., nivolumab or pembrolizumab). In specific embodiments, a cancer being treated in accordance with the methods described herein is PD-L1-negative and refractory or unresponsive to monotherapy treatment with a PD-L1 blocking antibody (e.g., avelumab).

In specific embodiments, a cancer being treated in accordance with the methods described herein has low levels of PD-L1 expression and is refractory or unresponsive to monotherapy treatment with an antagonist of PD-1 or a ligand thereof. In a specific embodiment, a cancer being treated in accordance with the methods described herein has low levels of PD-L1 expression and is refractory or unresponsive to monotherapy treatment with a PD-1 blocking antibody (e.g., nivolumab or pembrolizumab). In specific embodiments, a cancer being treated in accordance with the methods described herein has low levels of PD-L1 expression and is refractory or unresponsive to monotherapy treatment with a PD-L1 blocking antibody (e.g., avelumab).

In specific embodiments, a cancer being treated in accordance with the methods described herein is relapsed. In a specific embodiment, a cancer being treated in accordance with the methods described herein is relapsed and refractory or unresponsive to monotherapy treatment with an antagonist of PD-1 or a ligand thereof. In another specific embodiment, a cancer being treated in accordance with the methods described herein is relapsed and refractory or unresponsive to monotherapy treatment with a PD-1 blocking antibody (e.g., nivolumab or pembrolizumab). In specific embodiments, a cancer being treated in accordance with the methods described herein is relapsed and refractory or unresponsive to monotherapy treatment with a PD-L1 blocking antibody (e.g., avelumab).

In specific embodiments, a cancer being treated in accordance with the methods described herein is PD-L1-negative and relapsed. In specific embodiments, a cancer being treated in accordance with the methods described herein has low levels of PD-L1 expression and is relapsed.

In a specific embodiment, a cancer being treated in accordance with the methods described herein is relapsed, PD-L1-negative and refractory or unresponsive to monotherapy treatment with an antagonist of PD-1 or a ligand thereof. In a specific embodiment, a cancer being treated in accordance with the methods described herein is relapsed, PD-L I-negative and refractory or unresponsive to monotherapy treatment with a PD-1 blocking antibody (e.g., nivolumab or pembrolizumab). In specific embodiments, a cancer being treated in accordance with the methods described herein is relapsed, PD-L1-negative and refractory or unresponsive to monotherapy treatment with a PD-L1 blocking antibody (e.g., avelumab).

In specific embodiments, a cancer being treated in accordance with the methods described herein has low levels of PD-L1 expression, and is relapsed and refractory or unresponsive to monotherapy treatment with an antagonist of PD-1 or a ligand thereof. In a specific embodiment, a cancer being treated in accordance with the methods described herein has low levels of PD-L1 expression, and is relapsed and is refractory or unresponsive to monotherapy treatment with a PD-1 blocking antibody (e.g., nivolumab or pembrolizumab). In specific embodiments, a cancer being treated in accordance with the methods described herein has low levels of PD-L1 expression, and is relapsed and is refractory or unresponsive to monotherapy treatment with a PD-L1 blocking antibody (e.g., avelumab).

In specific embodiments, a cancer being treated in accordance with the methods described herein is refractory or unresponsive to monotherapy treatment with an antagonist of PD-1 or a ligand thereof. In a specific embodiment, a cancer being treated in accordance with the methods described herein is refractory or unresponsive to monotherapy treatment with a PD-1 blocking antibody (e.g., nivolumab or pembrolizumab). In specific embodiments, a cancer being treated in accordance with the methods described herein is refractory or unresponsive to monotherapy treatment with a PD-L1 blocking antibody (e.g., avelumab).

In a specific embodiment, the cancer being treated in accordance with the methods described herein is a cancer that is metastatic. In a specific embodiment, the cancer comprises a dermal, subcutaneous, or nodal metastasis. In a specific embodiment, the cancer comprises peritoneal or pleural metastasis. In a specific embodiment, the cancer comprises visceral organ metastasis, such as liver, kidney, spleen, or lung metastasis.

In a specific embodiment, the cancer being treated in accordance with the methods described herein is relapsed/refractory solid tumor types, such as melanoma, sarcoma, squamous cell cancer of the head and neck (SSCHN), breast carcinoma with dermal metastases and other malignancies with accessible dermal/SC/nodal metastases

In a specific embodiment, the cancer being treated in accordance with the methods described herein is a cancer that is unresectable. Any method known to the skilled artisan may be utilized to determine if a cancer is unresectable.

### 5.7.6 Additional Therapies

Additional therapies that can be used in a combination with an NDV described herein or a composition thereof, an oncolysate vaccine, or a whole cell vaccine for the treatment of cancer include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. In a specific embodiment, the additional therapy is a chemotherapeutic agent.

In some embodiments, an NDV described herein or a composition thereof, an oncolysate vaccine, or a whole cell vaccine is used in combination with radiation therapy comprising the use of x-rays, gamma rays and other sources of radiation to destroy cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer cells and/or a tumor mass.

In certain embodiments, an NDV described herein or a composition thereof, an oncolysate vaccine, or a whole cell cancer vaccine is used in combination with adoptive T cell therapy. In a specific embodiment, the T cells utilized in the adoptive T cell therapy are tumor infiltrating lymphocytes that have been isolated from a subject and a particular T cell or clone has been expanded for use thereof. In some embodiments, the T cells utilized in the adoptive T cell therapy are T cells taken from a patient's blood after they have received a cancer vaccine and expanded in vitro before use. In another specific embodiment, the T cells utilized in the adoptive T cell therapy are T cells that have been influenced to potently recognize and attack tumors. In another specific embodiment, the T cells utilized in the adoptive T cell therapy have been genetically modified to express tumor-antigen specific T cell receptor or a chimeric antigen receptor (CAR). In a specific embodiment, the adoptive T cell therapy utilized is analogous to that described in Section 6.2 of International Publication No. WO 2014/158811 and U.S. Patent Application Publication No. 2016/0015760, each of which is incorporated by reference herein in its entirety.

In certain embodiments, an NDV described herein or a composition thereof, an oncolysate vaccine, or a whole cell cancer vaccine is used in combination with a cytokine. In a specific embodiment, an NDV described herein or a composition thereof, an oncolysate vaccine, or a whole cell cancer vaccine is used in combination with interferon (e.g., IFN-γ).

Currently available cancer therapies and their dosages, routes of administration and recommended usage are known in the art and have been described in such literature as the Physician's Desk Reference (67th ed., 2013).

Specific examples of anti-cancer agents that may be used in combination with an NDV described herein or a composition thereof include: hormonal agents (e.g., aromatase inhibitor, selective estrogen receptor modulator (SERM), and estrogen receptor antagonist), chemotherapeutic agents (e.g., microtubule disassembly blocker, antimetabolite, topoisomerase inhibitor, and DNA crosslinker or damaging agent), anti-angiogenic agents (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that may be used in combination with an NDV described herein or a composition thereof include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that may be used in combination with an NDV described herein or a composition thereof, an oncolysate vaccine, or a whole cell vaccine include microtubule disassembly blocker, antimetabolite, topoisomerase inhibitor, and DNA crosslinker or damaging agent. Chemotherapeutic agents that are microtubule disassembly blockers include, but are not limited to, taxenes (e.g., paclitaxel (branded/marketed as TAXOLR), docetaxel, abraxane, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapeutic agents that are antimetabolites include, but are not limited to, folate antimetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capecitabine, gemcitabine (GEMZARR), cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATINR), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

### 5.7.6.1 Immune Modulators

In specific embodiments, an NDV described herein (e.g., a chimeric NDV) or a composition thereof, an oncolysate vaccine, or a whole cell vaccine are administered to a subject in combination with one or more of the following: any agonist of a co-stimulatory signal of an immune cell (such as, e.g., a T-lymphocyte, NK cell or antigen-presenting cell (e.g., a dendritic cell or macrophage) and/or any antagonist of an inhibitory signal of an immune cell (such as, e.g., a T-lymphocyte, NK cell or antigen-presenting cell (e.g., a dendritic cell or macrophage), known to one of skill in the art.

In specific embodiments, the agonist and/or antagonist is an agonist of a human co-stimulatory signal of an immune cell and/or antagonist of a human inhibitory signal of an immune cell.

In certain embodiments, the agonist of a co-stimulatory signal is an agonist of a co-stimulatory molecule (e.g., co-stimulatory receptor) found on immune cells, such as, e.g., T-lymphocytes (e.g., CD4+ or CD8+T-lymphocytes), NK cells and/or antigen-presenting cells (e.g., dendritic cells or macrophages). Specific examples of co-stimulatory molecules include glucocorticoid-induced tumor necrosis factor receptor (GITR), Inducible T-cell costimulator (ICOS or CD278), OX40 (CD134), CD27, CD28, 4-1BB (CD137), CD40, lymphotoxin alpha (LT alpha), LIGHT (lymphotoxin-like, exhibits inducible expression, and competes with herpes simplex virus glycoprotein D for HVEM, a receptor expressed by T lymphocytes), CD226, cytotoxic and regulatory T cell molecule (CRTAM), death receptor 3 (DR3), lymphotoxin-beta receptor (LTBR), transmembrane activator and CAML interactor (TACI), B cell-activating factor receptor (BAFFR), and B cell maturation protein (BCMA). In specific embodiments, the agonist is an agonist of a human co-stimulatory receptor of an immune cell. In certain embodiments, the agonist of a co-stimulatory receptor is not an agonist of ICOS.

In a specific embodiment, the agonist of a co-stimulatory receptor is an antibody or antigen-binding fragment thereof that specifically binds to the co-stimulatory receptor. Specific examples of co-stimulatory receptors include GITR, ICOS, OX40, CD27, CD28, 4-1BB, CD40, LT alpha, LIGHT, CD226, CRTAM, DR3, LTBR, TACI, BAFFR, and BCMA. In certain specific embodiments, the antibody is a monoclonal antibody. In other specific embodiments, the antibody is an sc-Fv. In other specific embodiments, the antibody is a camelized antibody. In a specific embodiment, the antibody is a bispecific antibody that binds to two receptors on an immune cell. In other embodiments, the bispecific antibody binds to a receptor on an immune cell and to another receptor on a cancer cell. In specific embodiments, the antibody is a human or humanized antibody. In some embodiments, the antibody is expressed as a chimeric protein with NDV F protein or fragment thereof, or NDV HN protein or fragment thereof. See, e.g., U.S. patent application Publication No. 2012/0122185, which is incorporated herein by reference for a description regarding generation of chimeric F or chimeric HN proteins.

In another embodiment, the agonist of a co-stimulatory receptor is a ligand of the co-stimulatory receptor. In certain embodiments, the ligand is fragment of a native ligand. Specific examples of native ligands include ICOSL, B7RP1, CD137L, OX40L, CD70, herpes virus entry mediator (HVEM), CD80, and CD86. The nucleotide sequences encoding native ligands as well as the amino acid sequences of native ligands are known in the art. For example, the nucleotide and amino acid sequences of B7RP1 (otherwise known as ICOSL; GenBank human: NM_015259.4, NP 056074.1 murine: NM_015790.3, NP 056605.1), CD137L (GenBank human: NM_003811.3, NP 003802.1, murine: NM_009404.3, NP 033430.1), OX40L (GenBank human: NM_003326.3, NP_003317.1, murine: NM_009452.2, NP_033478.1), CD70 (GenBank human: NM_001252.3, NP_001243.1, murine: NM_011617.2, AAD00274.1), CD80 (GenBank human: NM_005191.3, NP_005182.1, murine: NM_009855.2, NP_033985.3), and CD86 (GenBank human: NM_005191.3, CAG46642.1, murine: NM_019388.3, NP_062261.3) can be found in GenBank. In other embodiments, the ligand is a derivative of a native ligand. In some embodiments, the ligand is a fusion protein comprising at least a portion of the native ligand or a derivative of the native ligand that specifically binds to the co-stimulatory receptor, and a heterologous amino acid sequence. In specific embodiments, the fusion protein comprises at least a portion of the native ligand or a derivative of the native ligand that specifically binds to the co-stimulatory receptor, and the Fc portion of an immunoglobulin or a fragment thereof. An example of a ligand fusion protein is a 4-1BB ligand fused to Fc portion of immunoglobulin (described by Meseck M et al., J Immunother. 2011 34:175-82).

In some embodiments, the antagonist is an antagonist of an inhibitory molecule (e.g., inhibitory receptor) found on immune cells, such as, e.g., T-lymphocytes (e.g., CD4+ or CD8+T-lymphocytes), NK cells and/or antigen-presenting cells (e.g., dendritic cells or macrophages). Specific examples of inhibitory molecules include cytotoxic T-lymphocyte-associated antigen 4 (CTLA-4 or CD52), programmed cell death protein 1 (PD-1 or CD279), B and T-lymphocyte attenuator (BTLA), killer cell immunoglobulin-like receptor (KIR), lymphocyte activation gene 3 (LAG3), T-cell membrane protein 3 (TIM3), CD160, adenosine A2a receptor (A2aR), T cell immunoreceptor with immunoglobulin and ITIM domains (TIGIT), leukocyte-associated immunoglobulin-like receptor 1 (LAIR1), and CD160. In specific embodiments, the antagonist is an antagonist of a human inhibitory receptor of an immune cell. In a specific embodiment, the antagonist of an inhibitory molecule is an antagonist of PD-1 or a ligand thereof (such as, e.g., as described in Section 5.5, supra).

In another embodiment, the antagonist of an inhibitory receptor is an antibody (or an antigen-binding fragment) or a soluble receptor that specifically binds to the native ligand for the inhibitory receptor and blocks the native ligand from binding to the inhibitory receptor and transducing an inhibitory signal(s). Specific examples of native ligands for inhibitory receptors include PDL-1, PDL-2, B7-H3, B7-H4, HVEM, Gal9 and adenosine. Specific examples of inhibitory receptors that bind to a native ligand include CTLA-4, PD-1, BTLA, KIR, LAG3, TIM3, and A2aR.

In specific embodiments, the antagonist of an inhibitory receptor is a soluble receptor that specifically binds to the native ligand for the inhibitory receptor and blocks the native ligand from binding to the inhibitory receptor and transducing an inhibitory signal(s). In certain embodiments, the soluble receptor is a fragment of a native inhibitory receptor or a fragment of a derivative of a native inhibitory receptor that specifically binds to native ligand (e.g., the extracellular domain of a native inhibitory receptor or a derivative of an inhibitory receptor). In some embodiments, the soluble receptor is a fusion protein comprising at least a portion of the native inhibitory receptor or a derivative of the native inhibitory receptor (e.g., the extracellular domain of the native inhibitory receptor or a derivative of the native inhibitory receptor), and a heterologous amino acid sequence. In specific embodiments, the fusion protein comprises at least a portion of the native inhibitory receptor or a derivative of the native inhibitory receptor, and the Fc portion of an immunoglobulin or a fragment thereof. An example of a soluble receptor fusion protein is a LAG3-Ig fusion protein (described by Huard B et al., Eur J Immunol. 1995 25:2718-21).

In specific embodiments, the antagonist of an inhibitory receptor is an antibody (or an antigen-binding fragment) that specifically binds to the native ligand for the inhibitory receptor and blocks the native ligand from binding to the inhibitory receptor and transducing an inhibitory signal(s). In certain specific embodiments, the antibody is a monoclonal antibody. In other specific embodiments, the antibody is an scFv. In particular embodiments, the antibody is a human or humanized antibody. A specific example of an antibody to inhibitory ligand is anti-PD-L1 antibody (Iwai Y, et al. PNAS 2002; 99:12293-12297).

In another embodiment, the antagonist of an inhibitory receptor is an antibody (or an antigen-binding fragment) or ligand that binds to the inhibitory receptor, but does not transduce an inhibitory signal(s). Specific examples of inhibitory receptors include CTLA-4, PD-1, BTLA, KIR, LAG3, TIM3, and A2aR. In certain specific embodiments, the antibody is a monoclonal antibody. In other specific embodiments, the antibody is an scFv. In particular embodiments, the antibody is a human or humanized antibody. A specific example of an antibody to inhibitory receptor is anti-CTLA-4 antibody (Leach D R, et al. Science 1996; 271:1734-1736). Another example of an antibody to inhibitory receptor is anti-PD-1 antibody (Topalian S L, NEJM 2012; 28:3167-75).

In certain embodiments, an antagonist of an inhibitory receptor is an antagonist of CTLA-4, such as, e.g., Ipilimumab or Tremelimumab. In certain embodiments, the antagonist of an inhibitory receptor is an antagonist of PD-1, such as, e.g., MDX-1106 (BMS-936558), MK3475, CT-011, AMP-224, or MDX-1105. In certain embodiments, an antagonist of an inhibitory receptor is an antagonist of LAG3, such as, e.g., IMP321. In certain embodiments, an antagonist of a inhibitory receptor is an antibody (e.g., a monoclonal antibody or an antigen-binding fragment thereof, or scFv) that binds to B7-H3, such as, e.g., MGA271. In specific embodiments, an agonist of a co-stimulatory receptor is anti-CD28 scvFv, ICOSL, CD40L, OX40L, CD137L, GITRL, and/or CD70.

In certain embodiments, an agonist of a co-stimulatory signal of an immune cell induces (e.g., selectively) induces one or more of the signal transduction pathways induced by the binding of a co-stimulatory receptor to its ligand. In specific embodiments, an agonist of a co-stimulatory receptor induces one or more of the signal transduction pathways induced by the binding of the co-stimulatory receptor to one or more of its ligands by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of the co-stimulatory receptor to one or more of its ligands in the absence of the agonist. In specific embodiments, an agonist of a co-stimulatory receptor: (i) induces one or more of the signal transduction pathways induced by the binding of the co-stimulatory receptor to one particular ligand by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of the co-stimulatory receptor to the particular ligand in the absence of the agonist; and (ii) does not induce, or induces one or more of the signal transduction pathways induced by the binding of the co-stimulatory receptor to one or more other ligands by less than 20%, 15%, 10%, 5%, or 2%, or in the range of between 2% to 5%, 2% to 10%, 5% to 10%, 5% to 15%, 5% to 20%, 10% to 15%, or 15% to 20% relative to the one or more signal transduction pathways induced by the binding of the co-stimulatory receptor to such one or more other ligands in the absence of the agonist.

In certain embodiments, an agonist of a co-stimulatory signal of an immune cell activates or enhances (e.g., selectively activates or enhances) one or more of the signal transduction pathways induced by the binding of a co-stimulatory receptor to its ligand. In specific embodiments, an agonist of a co-stimulatory receptor activates or enhances one or more of the signal transduction pathways induced by the binding of the co-stimulatory receptor to one or more of its ligands by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of co-stimulatory receptor to one or more of its ligands in the absence of the agonist. In specific embodiments, an agonist of a co-stimulatory receptor: (i) an agonist of a co-stimulatory signal activates or enhances one or more of the signal transduction pathways induced by the binding of the co-stimulatory receptor to one particular ligand by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of the co-stimulatory receptor to the particular ligand in the absence of the agonist; and (ii) does not activate or enhance, or activates or enhances one or more of the signal transduction pathways induced by the binding of the co-stimulatory receptor to one or more other ligands by less than 20%, 15%, 10%, 5%, or 2%, or in the range of between 2% to 5%, 2% to 10%, 5% to 10%, 5% to 15%, 5% to 20%, 10% to 15%, or 15% to 20% relative to the one or more signal transduction pathways induced by the binding of the co-stimulatory receptor to such one or more other ligands in the absence of the agonist.

In some embodiments, an antagonist of an inhibitory signal of an immune cell (e.g., selectively) inhibits or reduces one or more of the signal transduction pathways induced by the binding of an inhibitory receptor to its ligand. In specific embodiments, an antagonist of an inhibitory receptor inhibits or reduces one or more of the signal transduction pathways induced by the binding of the inhibitory receptor to one or more of its ligands by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of the inhibitory receptor to one or more of its ligands in the absence of the antagonist. In specific embodiments, an antagonist of an inhibitory receptor: (i) inhibits or reduces one or more of the signal transduction pathways induced by the binding of the inhibitory receptor to one particular ligand by at least 25%, 30%, 40%, 50%, 60%, 75%, 80%, 85%, 90%, 95%, 98% or 99%, or in the range of between 25% to 50%, 25% to 75%, 50% to 75%, 50% to 95%, 75% to 95%, or 75% to 100% relative to the one or more signal transduction pathways induced by the binding of the inhibitory receptor to the one particular ligand in the absence of the antagonist; and (ii) does not inhibit or reduce, or inhibits or reduces one or more of the signal transduction pathways induced by the binding of the inhibitory receptor to one or more other ligands by less than 20%, 15%, 10%, 5%, or 2%, or in the range of between 2% to 5%, 2% to 10%, 5% to 10%, 5% to 15%, 5% to 20%, 10% to 15%, or 15% to 20% relative to the one or more signal transduction pathways induced by the binding of inhibitory receptor to such one or more other ligands in the absence of the antagonist.

In specific embodiments, an agonist of a co-stimulatory signal of an immune cell and/or an antagonist of an inhibitory signal of an immune cell induces, activates and/or enhances one or more immune activities, functions or responses. The one or more immune activities, functions or responses can be in the form of, e.g., an antibody response (humoral response) or a cellular immune response, e.g., cytokine secretion (e.g., interferon-gamma), helper activity or cellular cytotoxicity. In one embodiment, expression of an activation marker on immune cells (e.g., CD44, Granzyme, or Ki-67), expression of a co-stimulatory receptor on immune cells (e.g., ICOS, CD28, OX40, or CD27), expression of a ligand for a co-stimulatory receptor (e.g., B7HRP1, CD80, CD86, OX40L, or CD70), cytokine secretion, infiltration of immune cells (e.g., T-lymphocytes, B lymphocytes and/or NK cells) to a tumor, antibody production, effector function, T cell activation, T cell differentiation, T cell proliferation, B cell differentiation, B cell proliferation, and/or NK cell proliferation is induced, activated and/or enhanced following contact with an agonist of a co-stimulatory signal of an immune cell and/or an antagonist of an inhibitory signal of an immune cell. In another embodiment, myeloid-derived suppressor cell (MDSC) tumor infiltration and proliferation, Treg tumor infiltration, activation and proliferation, peripheral blood MDSC and Treg counts are inhibited following contact with an agonist of a co-stimulatory signal of an immune cell and/or an antagonist of an inhibitory signal of an immune cell.

### 5.8 Biological Assays

In certain embodiments, an assay described in Section 6, infra, is used to characterize/assess, e.g., the production of a chimeric NDV, the expression, function or both of IL-12 expressed by a chimeric NDV, or the efficacy of a method described herein.

### 5.8.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the NDVs described herein can be assessed by any method known in the art or described herein (e.g., in cell culture (e.g., cultures of chicken embryonic kidney cells or cultures of chicken embryonic fibroblasts (CEF)) (see, e.g., Section 6). Viral titer may be determined by inoculating serial dilutions of a NDV described herein into cell cultures (e.g., CEF, MDCK, EFK-2 cells, Vero cells, primary human umbilical vein endothelial cells (HUVEC), H292 human epithelial cell line or HeLa cells), chick embryos, or live animals (e.g., avians). After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50). An exemplary method of assessing viral titer is described in Section 6, below.

Incorporation of nucleotide sequences encoding a heterologous peptide or protein (e.g., a cytokine, a mutated F protein, a mutated V protein, or miRNA target site into the genome of a chimeric NDV described herein can be assessed by any method known in the art or described herein (e.g., in cell culture, an animal model or viral culture in embryonated eggs). For example, viral particles from cell culture of the allantoic fluid of embryonated eggs can be purified by centrifugation through a sucrose cushion and subsequently analyzed for fusion protein expression by Western blotting using methods well known in the art.

Immunofluorescence-based approaches may also be used to detect virus and assess viral growth. Such approaches are well known to those of skill in the art, e.g., fluorescence microscopy and flow cytometry (see Section 6, infra). Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

### 5.8.2 IFN Assays

IFN induction and release by an NDV described herein may be determined using techniques known to one of skill in the art or described herein (see, e.g., Section 6). For example, the amount of IFN induced in cells following infection with an NDV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. (See, e.g., the methods described in Section 6, below.). Further, the induction and release of cytokines and/or interferon-stimulated genes may be determined by, e.g., an immunoassay or ELISPOT assay at the protein level and/or quantitative RT-PCR or northern blots at the RNA level. See Section 6, infra, regarding assays to measure cytokine and/or interferon-stimulated gene induction and release.

### 5.8.3 Activation Marker Assays

Techniques for assessing the expression of activation marker, co-stimulatory molecule, ligand, or inhibitory molecule by immune cells are known to one of skill in the art. For example, the expression of an activation marker, co-stimulatory molecule, ligand, or inhibitory molecule by an immune cell (e.g., T lymphocyte or NK cell) can be assessed by flow cytometry. In a specific embodiment, techniques described in Section 6, infra, are used to assess the expression of an activation marker, co-stimulatory molecule, ligand, or inhibitory molecule by an immune cell.

### 5.8.4 Immune Cell Infiltration Assays

Techniques for assessing immune cell infiltration are known to one of skill in the art. In a specific embodiment, techniques described in Section 6, infra, are used to assess immune cell infiltration.

### 5.8.5 Toxicity Studies

In some embodiments, the NDVs described herein or compositions thereof, oncolysate vaccines described herein, whole cell vaccines described herein, or combination therapies described herein are tested for cytotoxicity in mammalian, preferably human, cell lines (see, e.g., the cytotoxicity assay described in Section 6, infra). In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In certain embodiments, cytotoxicity is assessed in various cancer cells. In some embodiments, the ToxLite assay is used to assess cytotoxicity.

Many assays well-known in the art can be used to assess viability of cells or cell lines following infection with an NDV described herein or composition thereof, or treatment with an oncolysate vaccine described herein, a whole cell vaccine described herein, or a combination therapy described herein and, thus, determine the cytotoxicity of the NDV or composition thereof, oncolysate vaccine, whole cell vaccine, or combination therapy. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, an NDV described herein or composition thereof, oncolysate vaccine, whole cell vaccine, or combination therapy kills cancer cells but does not kill healthy (i.e., non-cancerous) cells. In one embodiment, an NDV described herein or composition thereof, oncolysate vaccine, whole cell vaccine, or combination therapy preferentially kills cancer cells but does not kill healthy (i.e., non-cancerous) cells.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The NDVs described herein or compositions thereof, oncolysate vaccines, whole cell vaccines or combination therapies can be tested for in vivo toxicity in animal models (see, e.g., the animal models described in Section 6, below). For example, animal models, described herein and/or others known in the art, used to test the effects of compounds on cancer can also be used to determine the in vivo toxicity of the NDVs described herein or compositions thereof, oncolysate vaccines, whole cell vaccines, or combination therapies. For example, animals are administered a range of pfu of an NDV described herein (e.g., a chimeric NDV described in Section 5.2, infra). Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of an NDV described herein or a composition thereof, an oncolysate vaccine described herein, a whole cell vaccine described herein, or a combination therapy described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibits large therapeutic indices are preferred. While therapies that exhibits toxic side effects may be used, care should be taken to design a delivery system that targets such therapies to the site of affected tissue in order to minimize potential damage to noncancerous cells and, thereby, reduce side effects.

The data obtained from the cell culture assays and animal studies can be used in formulating a range of dosage of the therapies for use in subjects. The dosage of such agents lies preferably within a range of circulating concentrations that include the ED50 with little or no toxicity. The dosage may vary within this range depending upon the dosage form employed and the route of administration utilized. For any therapy described herein, the therapeutically effective dose can be estimated initially from cell culture assays. A dose may be formulated in animal models to achieve a circulating plasma concentration range that includes the IC50 (i.e., the concentration of the chimeric NDV that achieves a half-maximal inhibition of symptoms) as determined in cell culture. Such information can be used to more accurately determine useful doses in subjects. Levels in plasma may be measured, for example, by high performance liquid chromatography.

### 5.8.6 Anti-Cancer Studies

The NDVs described herein or compositions thereof, oncolysate vaccines described herein, whole cell vaccines described herein, or combination therapies described herein can be tested for biological activity using animal models for cancer (see, e.g., Section 6). Such animal model systems include, but are not limited to, rats, mice, chicken, cows, monkeys, pigs, dogs, rabbits, etc. In a specific embodiment, the anti-cancer activity of an NDV described herein or combination therapy is tested in a mouse model system. Such model systems are widely used and well-known to the skilled artisan such as the SCID mouse model or transgenic mice.

The anti-cancer activity of an NDV described herein or a composition thereof, oncolysate vaccine described herein, whole cell vaccine described herein, or a combination therapy described herein can be determined by administering the NDV or composition thereof, oncolysate vaccine, whole cell vaccine, or combination therapy to an animal model and verifying that the NDV or composition thereof, oncolysate vaccine, whole cell vaccine, or combination therapy is effective in reducing the severity of cancer, reducing the symptoms of cancer, reducing cancer metastasis, and/or reducing the size of a tumor in said animal model (see, e.g., Section 6, below). Examples of animal models for cancer in general include, include, but are not limited to, animal models refractory or unresponsive to treatment with an antagonist of PD-1 or a ligand thereof, such as, e.g., the B16F10 mouse model (e.g., as described in Section 6), and spontaneously occurring tumors of companion animals (see, e.g., Vail & MacEwen, 2000, Cancer Invest 18 (8): 781-92). Examples of animal models for lung cancer include, but are not limited to, lung cancer animal models described by Zhang & Roth (1994, In-vivo 8 (5): 755-69) and a transgenic mouse model with disrupted p53 function (see, e.g.e.g., Morris et al., 1998, J La State Med Soc 150 (4): 179-85). An example of an animal model for breast cancer includes, but is not limited to, a transgenic mouse that over expresses cyclin D1 (see, e.g., Hosokawa et al., 2001, Transgenic Res 10 (5): 471-8). An example of an animal model for colon cancer includes, but is not limited to, a TCR b and p53 double knockout mouse (see, e.g., Kado et al., 2001, Cancer Res. 61 (6): 2395-8). Examples of animal models for pancreatic cancer include, but are not limited to, a metastatic model of PancO2 murine pancreatic adenocarcinoma (see, e.g., Wang et al., 2001, Int. J. Pancreatol. 29 (1): 37-46) and nu-nu mice generated in subcutaneous pancreatic tumors (see, e.g., Ghaneh et al., 2001, Gene Ther. 8 (3): 199-208). Examples of animal models for non-Hodgkin lymphoma include, but are not limited to, a severe combined immunodeficiency (“SCID”) mouse (see, e.g., Bryant et al., 2000, Lab Invest 80 (4): 553-73) and an IgHmu-HOX11 transgenic mouse (see, e.g., Hough et al., 1998, Proc. Natl. Acad. Sci. USA 95 (23): 13853-8). An example of an animal model for esophageal cancer includes, but is not limited to, a mouse transgenic for the human papillomavirus type 16 E7 oncogene (see, e.g., Herber et al., 1996, J. Virol. 70 (3): 1873-81). Examples of animal models for colorectal carcinomas include, but are not limited to, Apc mouse models (see, e.g., Fodde & Smits, 2001, Trends Mol Med 7 (8): 369 73 and Kuraguchi et al., 2000) and ID8 ovarian cancer model. In a specific embodiment, the animal models for cancer described in Section 6, infra, are used to assess efficacy of an NDV or composition thereof, an oncolysate, a whole cell vaccine, or a combination therapy.

### 5.8.7 Expression of IL-12

Assays for testing the expression of IL-12 or a derivative thereof in cells infected with a chimeric NDV comprising a packaged genome comprising a transgene encoding IL-12 or a derivative thereof may be conducted using any assay known in the art, such as, e.g., western blot, immunofluorescence, and ELISA, or any assay described herein (see, e.g., Section 6).

In a specific aspect, ELISA is utilized to detect expression of IL-12 or a derivative thereof in cells infected with a chimeric NDV comprising a packaged genome comprising a transgene encoding IL-12 or a derivative thereof. For example, cells (e.g., Vero cells) are seeded in a tissue culture plate (e.g., a 96-well plate) at the appropriate concentration (e.g., 1×10⁴ vero cells/well of a 96-well plate) in serum free medium (e.g., OptiPRO serum free medium (Gibco, Cat. No. 12309-019) supplemented with 2% glutamine (e.g., Corning, Cat. No. 25-005-CI) and incubated under standard conditions (e.g., 37±2° C., 5±2% CO₂) for a period of time (e.g., approximately 24 hours). Test samples of the chimeric NDV are pre-diluted in reduced serum medium (e.g., Opti-MEM (1λ) reduced serum medium (Gibco, Cat. No. 31985-070)) to a desired titer (e.g., 2×10⁴ pfu/mL). A volume of the pre-diluted test sample (e.g., 300 μL) is added to, e.g., a row of, a 0.5 mL Assay Block (Costar, Cat. No. 3956) and serial dilutions (e.g., 2-fold serial dilutions) are performed across rows by transferring a volume of sample into a volume of reduced serum medium (e.g., 150 μL of sample into 150 μL Opti-MEM (1×) reduced serum medium). Two replicates per sample may be prepared. The tissue culture plates (e.g., 96-well plates) containing the cells (e.g., Vero cells) are removed from the incubator a period of time after seeding (e.g., approximately 24 hours post seeding) and spent medium is removed from the plate. The cells are inoculated with a volume (e.g., 100 μL) of the serially diluted test samples and incubated under standard conditions (e.g., 37° C., 5% CO₂). After a period of time of incubation (e.g., approximately 24 hours), a volume of the supernatant fluid (e.g., 90 μL) is removed from the infection plates, transferred to an ELISA plate pre-coated with anti-human IL-12 p70 capture antibody (e.g., Affymetrix eBioscience, Cat. No. 14-7128-68) and incubated for a period of time (e.g., two hours at room temperature). The captured hulL-12 is detected with anti-human IL-12 p70 detection antibody (e.g., Affymetrix eBioscience, Cat. No. 33-8261-68A) and, e.g., Avidin-HRP and visualized with, e.g., a HRP substrate TMB following vendor's procedure (Affymetrix eBioscience, Human IL-12 p70 ELISA Ready-SET-Go! ELISA kit, Cat. No. 88-7126-88). In a specific embodiment, human IL-12 is quantitied using an ELISA such as described in Section 6.3.1.19, infra.

In one embodiment, an IL-12 or a derivative thereof encoded by a packaged genome of a chimeric NDV described herein is assayed for proper folding and functionality by testing its ability to bind specifically to an anti-IL-12 antibody using any assay for antibody-antigen interaction known in the art. In another embodiment, an IL-12 or a derivative thereof encoded by a packaged genome of a chimeric NDV described herein is assayed for proper folding by determination of the structure or conformation of the IL-12 or derivative thereof using any method known in the art such as, e.g., NMR, X-ray crystallographic methods, or secondary structure prediction methods, e.g., circular dichroism.

Assays for testing the functionality of IL-12 or a derivative thereof in cells infected with a chimeric NDV comprising a packaged genome comprising a transgene encoding IL-12 or a derivative thereof may be conducted using any assay known in the art, such as, e.g., PathHunter® Bioassay Detection Kit (DiscoverX, Cat #93-0933). For example, to evaluate the functionality of huIL-12 produced from a chimeric NDV comprising a packaged genome comprising a transgene encoding hull-12 or a derivative thereof in cells infected with the chimeric NDV, cells (e.g., vero cells) are seeded in plates (e.g., 5×10⁵ vero cells/well of a 6-well tissue culture plate) and incubated for a period of time, e.g., 24 hours, under standard conditions known to the skilled artisan for the cell type (e.g., 37° C., 5% CO₂). Test samples of the chimeric NDV are diluted (e.g., to 1×10⁶ pfu/mL) in reduced serum medium (e.g., Opti-MEM (1×) reduced serum medium) and various amounts of the diluted samples are transferred to the cell plate to target MOI between 0.03-1. Medium is then then added to each well to a final volume appropriate for the size of the tissue culture plate (e.g., 2 mL per well of a 6-well tissue culture plate). The infected cell plate is incubated at 37° C., 5% CO₂ for 24 hours and the function of the produced huIL-12 or derivative thereof in the supernatant is assayed using the PathHunter® Bioassay Detection kit (DiscoverX, Cat #93-0933) according to the manufacturer's instructions. Briefly, U2OS IL12RB1/IL12RB2 cells are seeded in a 96-well cell plate (e.g., at 5×10³ cells/well) and are incubated under standard conditions (e.g., 37° C., 5% CO₂) for a period of time (e.g., 4-6 hours). A volume of supernatant fluids (e.g., 60 μL) from each plate of the chimeric NDV-infected plate is transferred to a second column of a 96-well sample dilution plate and a 3-fold serial dilution in AssayComplete Cell Plating Reagent (DiscoverX, 93-0563R5A) is carried out. A portion (e.g., 10 μL) of each diluted supernatant is transferred to the U2OS cell plate and the plate is incubated under standard conditions (e.g., at 37° C., 5% CO₂) for a period of time (e.g., 16-20 hours). Detection Reagent 1 (e.g., 10 μL) is added to each well and the plate is incubated for a period of time (e.g., 15 minutes) at room temperature. Detection Reagent 2 (e.g., 40 μL) is added to each well and the plate is further incubated for a period of time (e.g., 60 minutes) at room temperature. The chemiluminescence signal is detected using a plate reader (e.g., a SpectraMax M5 plate reader). In a specific embodiment, the functionality of human IL-12 is assessed using an assay described in Section 6.3.1.18, infra.

### 5.9 Kits

In one aspect, provided herein is a pharmaceutical pack or kit comprising one or more containers filled with one or more of the ingredients of a composition (e.g., a pharmaceutical compositions) described herein. In a specific embodiment, provided herein is a pharmaceutical pack or kit comprising a first container and a second container, wherein the first container comprises an antagonist of PD-1 or a ligand thereof as described herein, or a pharmaceutical composition comprising the antagonist, and the second container comprises a chimeric NDV comprising a packaged genome comprising a transgene encoding IL-12 (e.g., human IL-12) or a derivative thereof, or a pharmaceutical composition comprising the chimeric NDV. In another specific embodiment, provided herein is a pharmaceutical pack or kit comprising a first container and a second container, wherein the first container comprises a PD-1 blocking antibody as described herein, or a pharmaceutical composition comprising the PD-1 blocking antibody, and the second container comprises a chimeric NDV comprising a packaged genome comprising a transgene encoding IL-12 (e.g., human IL-12) or a derivative thereof, or a pharmaceutical composition comprising the chimeric NDV. In one embodiment, the PD-1 blocking antibody is nivolumab. In a preferred embodiment, the PD-1 blocking antibody is pembrolizumab. In another specific embodiment, provided herein is a pharmaceutical pack or kit comprising a first container and a second container, wherein the first container comprises a PD-L1 blocking antibody as described herein, or a pharmaceutical composition comprising the PD-L1 blocking antibody, and the second container comprises a chimeric NDV comprising a packaged genome comprising a transgene encoding IL-12 (e.g., human IL-12) or a derivative thereof, or a pharmaceutical composition comprising the chimeric NDV. In certain embodiments, the PD-L1 blocking antibody is duralumab or avelumab. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.10 Sequences

TABLE 1
Exemplary p40 sequences (amino acid)

| | | SEQ ID |
|---|---|---|
| Name | Sequence | NO. |
| Version 1: | MGHQQLVISWFSLVFLASPLVAIWELKKDVYVVELDWY | 23 |
| human IL12B | PDAPGEMVVLTCDTPEEDGITWTLDQSSEVLGSGKTLTIQ | |
| (p40 subunit) | VKEFGDAGQYTCHKGGEVLSHSLLLLHKKEDGIWSTDILK | |
| with signal | DQKEPKNKTFLRCEAKNYSGRFTCWWLTTISTDLTFSVKS | |
| peptide (bold) | SRGSSDPQGVTCGAATLSAERVRGDNKEYEYSVECQEDS | |
| | ACPAAEESLPIEVMVDAVHKLKYENYTSSFFIRDIIKPDPPK | |
| | NLQLKPLKNSRQVEVSWEYPDTWSTPHSYFSLTFCVQVQ | |
| | GKSKREKKDRVFTDKTSATVICRKNASISVRAQDRYYSSS | |
| | WSEWASVPCS | |
| Version | IWELKKDVYVVELDWYPDAPGEMVVLTCDTPEEDGITWT | 38 |
| 1/Version | LDQSSEVLGSGKTLTIQVKEFGDAGQYTCHKGGEVLSHSL | |
| 2.1/Version 3: | LLLHKKEDGIWSTDILKDQKEPKNKTFLRCEAKNYSGRFT | |
| human IL12B | CWWLTTISTDLTFSVKSSRGSSDPQGVTCGAATLSAERVR | |
| (p40 subunit) | GDNKEYEYSVECQEDSACPAAEESLPIEVMVDAVHKLKY | |
| without signal | ENYTSSFFIRDIIKPDPPKNLQLKPLKNSRQVEVSWEYPDT | |
| peptide | WSTPHSYFSLTFCVQVQGKSKREKKDRVFTDKTSATVICR | |
| | KNASISVRAQDRYYSSSWSEWASVPCS | |
| Version | MCHQQLVISWFSLVFLASPLVAIWELKKDVYVVELDWY | 40 |
| 2.1/Version 3: | PDAPGEMVVLTCDTPEEDGITWTLDQSSEVLGSGKTLTIQ | |
| human IL12B | VKEFGDAGQYTCHKGGEVLSHSLLLLHKKEDGIWSTDILK | |
| (p40 subunit) | DQKEPKNKTFLRCEAKNYSGRFTCWWLTTISTDLTFSVKS | |
| with signal | SRGSSDPQGVTCGAATLSAERVRGDNKEYEYSVECQEDS | |
| peptide (bold) | ACPAAEESLPIEVMVDAVHKLKYENYTSSFFIRDIIKPDPPK | |
| | NLQLKPLKNSRQVEVSWEYPDTWSTPHSYFSLTFCVQVQ | |
| | GKSKREKKDRVFTDKTSATVICRKNASISVRAQDRYYSSS | |
| | WSEWASVPCS | |

TABLE 2
Exemplary p40 sequences (nucleic acid)

| | | SEQ ID |
|---|---|---|
| Name | Sequence | NO. |
| Version 1: | ATGGGTCACCAGCAGTTGGTCATCTCTTGGTTTTCCCTG | 27 |
| nucleic acid | GTTTTTCTGGCATCTCCCCTCGTGGCCATATGGGAACTG | |
| sequence | AAGAAAGATGTTTATGTCGTAGAATTGGATTGGTATCC | |
| encoding human | GGATGCCCCTGGAGAAATGGTGGTCCTCACCTGTGACA | |
| IL12B (p40 | CCCCTGAAGAAGATGGTATCACCTGGACCTTGGACCAG | |
| subunit) with | AGCAGTGAGGTCTTAGGCTCTGGCAAAACCCTGACCAT | |
| signal peptide | CCAAGTCAAAGAGTTTGGAGATGCTGGCCAGTACACCT | |
| (exemplary | GTCACAAAGGAGGCGAGGTTCTAAGCCATTCGCTCCTG | |
| nucleic acid | CTGCTTCACAAAAAGGAAGATGGAATTTGGTCCACTGA | |
| sequence | TATTTTAAAGGACCAGAAAGAACCCAAAAATAAGACCT | |
| encoding SEQ | TTCTAAGATGCGAGGCCAAGAATTATTCTGGACGTTTCA | |
| ID NO: 23) | CCTGCTGGTGGCTGACGACAATCAGTACTGATTTGACAT | |
| | TCAGTGTCAAAAGCAGCAGAGGCTCTTCTGACCCCCAA | |
| | GGGGTGACGTGCGGAGCTGCTACACTCTCTGCAGAGAG | |
| | AGTCAGAGGGGACAACAAGGAGTATGAGTACTCAGTG | |
| | GAGTGCCAGGAGGACAGTGCCTGCCCAGCTGCTGAGGA | |
| | GAGTCTGCCCATTGAGGTCATGGTGGATGCCGTTCACA | |
| | AGCTCAAGTATGAAAACTACACCAGCAGCTTCTTCATC | |
| | AGGGACATCATCAAACCTGACCCACCCAAGAACTTGCA | |
| | GCTGAAGCCATTAAAGAATTCTCGGCAGGTGGAGGTCA | |
| | GCTGGGAGTACCCTGACACCTGGAGTACTCCACATTCCT | |
| | ACTTCTCCCTGACATTCTGCGTTCAGGTCCAGGGCAAGA | |
| | GCAAGAGAGAAAAGAAAGATAGAGTCTTCACGGACAA | |
| | GACCTCAGCCACGGTCATCTGCCGCAAAAATGCCAGCA | |
| | TTAGCGTGCGGGCCCAGGACCGCTACTATAGCTCATCTT | |
| | GGAGCGAATGGGCATCTGTGCCCTGCAGT | |
| Version | ATATGGGAACTGAAGAAAGATGTTTATGTCGTAGAATT | 57 |
| 1/Version 3: | GGATTGGTATCCGGATGCCCCTGGAGAAATGGTGGTCC | |
| nucleic acid | TCACCTGTGACACCCCTGAAGAAGATGGTATCACCTGG | |
| sequence | ACCTTGGACCAGAGCAGTGAGGTCTTAGGCTCTGGCAA | |
| encoding human | AACCCTGACCATCCAAGTCAAAGAGTTTGGAGATGCTG | |
| IL12B (p40 | GCCAGTACACCTGTCACAAAGGAGGCGAGGTTCTAAGC | |
| subunit) without | CATTCGCTCCTGCTGCTTCACAAAAAGGAAGATGGAAT | |
| signal peptide | TTGGTCCACTGATATTTTAAAGGACCAGAAAGAACCCA | |
| (exemplary | AAAATAAGACCTTTCTAAGATGCGAGGCCAAGAATTAT | |
| nucleic acid | TCTGGACGTTTCACCTGCTGGTGGCTGACGACAATCAGT | |
| sequence | ACTGATTTGACATTCAGTGTCAAAAGCAGCAGAGGCTC | |
| encoding SEQ | TTCTGACCCCCAAGGGGTGACGTGCGGAGCTGCTACAC | |
| ID NO: 38) | TCTCTGCAGAGAGAGTCAGAGGGGACAACAAGGAGTAT | |
| | GAGTACTCAGTGGAGTGCCAGGAGGACAGTGCCTGCCC | |
| | AGCTGCTGAGGAGAGTCTGCCCATTGAGGTCATGGTGG | |
| | ATGCCGTTCACAAGCTCAAGTATGAAAACTACACCAGC | |
| | AGCTTCTTCATCAGGGACATCATCAAACCTGACCCACCC | |
| | AAGAACTTGCAGCTGAAGCCATTAAAGAATTCTCGGCA | |
| | GGTGGAGGTCAGCTGGGAGTACCCTGACACCTGGAGTA | |
| | CTCCACATTCCTACTTCTCCCTGACATTCTGCGTTCAGG | |
| | TCCAGGGCAAGAGCAAGAGAGAAAAGAAAGATAGAGT | |
| | CTTCACGGACAAGACCTCAGCCACGGTCATCTGCCGCA | |
| | AAAATGCCAGCATTAGCGTGCGGGCCCAGGACCGCTAC | |
| | TATAGCTCATCTTGGAGCGAATGGGCATCTGTGCCCTGC | |
| | AGT | |
| Version 2.1: | ATGTGCCATCAGCAGCTGGTCATCTCATGGTTCTCCCTG | 54 |
| codon-optimized | GTGTTTCTGGCCTCACCTCTGGTCGCAATCTGGGAACTG | |
| nucleic acid | AAAAAGGATGTGTACGTGGTGGAGCTGGACTGGTATCC | |
| sequence | CGATGCCCCTGGCGAGATGGTGGTGCTGACCTGCGACA | |
| encoding human | CACCCGAGGAGGATGGCATCACCTGGACACTGGATCAG | |
| IL12B (p40 | AGCTCCGAGGTGCTGGGAAGCGGCAAGACCCTGACAAT | |
| subunit) with | CCAGGTGAAGGAGTTCGGCGACGCCGGCCAGTACACCT | |
| signal peptide | GTCACAAGGGAGGAGAGGTGCTGAGCCACTCCCTGCTG | |
| (exemplary | CTGCTGCACAAGAAGGAGGATGGCATCTGGTCCACAGA | |
| codon-optimized | CATCCTGAAGGATCAGAAGGAGCCAAAGAACAAGACC | |
| nucleic acid | TTCCTGCGGTGCGAGGCCAAGAATTATAGCGGCCGGTT | |
| sequence | CACCTGTTGGTGGCTGACCACAATCTCCACCGATCTGAC | |
| encoding SEQ | ATTTTCTGTGAAGTCTAGCAGGGGATCCTCTGACCCACA | |
| ID NO: 40) | GGGAGTGACATGCGGAGCAGCCACCCTGAGCGCCGAG | |
| | AGGGTGCGCGGCGATAACAAGGAGTACGAGTATTCCGT | |
| | GGAGTGCCAGGAGGACTCTGCCTGTCCAGCAGCAGAGG | |
| | AGTCCCTGCCTATCGAAGTGATGGTGGATGCCGTGCAC | |
| | AAGCTGAAGTACGAGAATTATACCAGCTCCTTCTTTATC | |
| | CGGGACATCATCAAGCCCGATCCCCCTAAGAACCTGCA | |
| | GCTGAAGCCTCTGAAGAATAGCAGACAGGTGGAGGTGT | |
| | CCTGGGAGTACCCTGACACCTGGAGCACACCACACTCC | |
| | TATTTCTCTCTGACCTTTTGCGTGCAGGTGCAGGGCAAG | |
| | TCCAAGCGGGAGAAGAAGGACAGAGTGTTCACCGATA | |
| | AGACATCTGCCACCGTGATCTGTAGAAAGAACGCCTCT | |
| | ATCAGCGTGAGGGCCCAGGACCGCTACTATTCTAGCTC | |
| | CTGGTCCGAGTGGGCCTCTGTGCCTTGCAGC | |
| Version 2.1: | atctgggaactgaaaaaggatgtgtacgtggtggagctggactggtatcccgatgcccctgg | 59 |
| nucleic acid | cgagatggtggtgctgacctgcgacacacccgaggaggatggcatcacctggacactgga | |
| sequence | tcagagctccgaggtgctgggaagcggcaagaccctgacaatccaggtgaaggagttcgg | |
| encoding human | cgacgccggccagtacacctgtcacaagggaggagaggtgctgagccactccctgctgct | |
| IL12B (p40 | gctgcacaagaaggaggatggcatctggtccacagacatcctgaaggatcagaaggagcc | |
| subunit) without | aaagaacaagaccttcctgcggtgcgaggccaagaattatagcggccggttcacctgttggt | |
| signal peptide | ggctgaccacaatctccaccgatctgacattttctgtgaagtctagcaggggatcctctgacc | |
| (exemplary | cacagggagtgacatgcggagcagccaccctgagcgccgagagggtgcgcggcgataa | |
| nucleic acid | caaggagtacgagtattccgtggagtgccaggaggactctgcctgtccagcagcagagga | |
| sequence | gtccctgcctatcgaagtgatggtggatgccgtgcacaagctgaagtacgagaattatacca | |
| encoding SEQ | gctccttctttatccgggacatcatcaagcccgatccccctaagaacctgcagctgaagcctc | |
| ID NO: 38) | tgaagaatagcagacaggtggaggtgtcctgggagtaccctgacacctggagcacaccac | |
| | actcctatttctctctgaccttttgcgtgcaggtgcagggcaagtccaagcgggagaagaag | |
| | gacagagtgttcaccgataagacatctgccaccgtgatctgtagaaagaacgcctctatcag | |
| | cgtgagggcccaggaccgctactattctagctcctggtccgagtgggcctctgtgccttgca | |
| | gc | |
| Version 3: (non- | atgtgtcaccagcagttggtcatctcttggttttccctggtttttctggcatctcccctcgtggcca | 64 |
| codon- | tatgggaactgaagaaagatgtttatgtcgtagaattggattggtatccggatgcccctggag | |
| optimized) | aaatggtggtcctcacctgtgacacccctgaagaagatggtatcacctggaccttggaccag | |
| nucleic acid | agcagtgaggtcttaggctctggcaaaaccctgaccatccaagtcaaagagtttggagatgc | |
| sequence | tggccagtacacctgtcacaaaggaggcgaggttctaagccattcgctcctgctgcttcaca | |
| encoding human | aaaaggaagatggaatttggtccactgatattttaaaggaccagaaagaacccaaaaataag | |
| IL12B (p40 | acctttctaagatgcgaggccaagaattattctggacgtttcacctgctggtggctgacgaca | |
| subunit) with | atcagtactgatttgacattcagtgtcaaaagcagcagaggctcttctgacccccaaggggtg | |
| signal peptide | acgtgcggagctgctacactctctgcagagagagtcagaggggacaacaaggagtatgag | |
| (exemplary non- | tactcagtggagtgccaggaggacagtgcctgcccagctgctgaggagagtctgcccattg | |
| codon-optimized | aggtcatggtggatgccgttcacaagctcaagtatgaaaactacaccagcagcttcttcatca | |
| nucleic acid | gggacatcatcaaacctgacccacccaagaacttgcagctgaagccattaaagaattctcgg | |
| sequence | caggtggaggtcagctgggagtaccctgacacctggagtactccacattcctacttctccctg | |
| encoding SEQ | acattctgcgttcaggtccagggcaagagcaagagagaaaagaaagatagagtcttcacgg | |
| ID NO: 40) | acaagacctcagccacggtcatctgccgcaaaaatgccagcattagcgtgcgggcccagg | |
| | accgctactatagctcatcttggagcgaatgggcatctgtgccctgcagt | |

TABLE 3
Exemplary p35 sequences (amino acid)

| | | SEQ ID |
|---|---|---|
| Name | Sequence | NO. |
| Version 1: | RNLPVATPDPGMFPCLHHSQNLLRAVSNMLQKARQTLEF | 25 |
| human IL12A | YPCTSEEIDHEDITKDKTSTVEACLPLELTKNESCLNSRETS | |
| (p35 subunit) | FITNGSCLASRKTSFMMALCLSSIYEDSKMYQVEFKTMNA | |
| | KLLMDPKRQIFLDQNMLAVIDELMQALNFNSETVPQKSSL | |
| | EEPDFYKTKIKLCILLHAFRIRAVTIDRVMSYLNAS | |
| Version | RNLPVATPDPGMFPCLHHSQNLLRAVSNMLQKARQTLEF | 41 |
| 2.1/Version 3: | YPCTSEEIDHEDITKDKTSTVEACLPLELTKNESCLNSRETS | |
| human IL12A | FITNGSCLASRKTSFMMALCLSSIYEDLKMYQVEFKTMNA | |
| (p35 subunit) | KLLMDPKRQIFLDQNMLAVIDELMQALNFNSETVPQKSSL | |
| | EEPDFYKTKIKLCILLHAFRIRAVTIDRVMSYLNAS | |

TABLE 4
Exemplary p35 sequences (nucleic acid)

| | | SEQ ID |
|---|---|---|
| Name | Sequence | NO. |
| Version 1: | agaaacctccccgtggccactccagacccaggaatgttcccatgccttcaccactcccaaaa | 29 |
| nucleic acid | cctgctgagggccgtcagcaacatgctccagaaggccagacaaactctagaattttaccctt | |
| sequence | gcacttctgaagagattgatcatgaagatatcacaaaagataaaaccagcacagtggaggc | |
| encoding human | ctgtttaccattggaattaaccaagaatgagagttgcctaaattccagagagacctctttcataa | |
| IL12A (p35 | ctaatgggagttgcctggcctccagaaagacctcttttatgatggccctgtgccttagtagtatt | |
| subunit) | tatgaagactcgaagatgtaccaggtggagttcaagaccatgaatgcaaagcttctgatggat | |
| (exemplary | cctaagaggcagatctttctagatcaaaacatgctggcagttattgatgagctgatgcaggcc | |
| nucleic acid | ctgaatttcaacagtgagactgtgccacaaaaatcctcccttgaagaaccggatttttataaaa | |
| sequence | ctaaaatcaagctctgcatacttcttcatgctttcagaattcgggcagtgactattgatagagtg | |
| encoding SEQ | atgagctatctgaatgcttcctaa | |
| ID NO: 25) | | |
| Version 2.1: | aggaatctgccagtggcaacccctgacccaggcatgttcccctgcctgcaccacagccaga | 55 |
| codon-optimized | acctgctgagggccgtgtccaatatgctgcagaaggcccgccagacactggagttttaccct | |
| nucleic acid | tgtaccagcgaggagatcgaccacgaggacatcacaaaggataagacctccacagtggag | |
| sequence | gcctgcctgccactggagctgaccaagaacgagtcctgtctgaacagccgggagacaagc | |
| encoding human | ttcatcaccaacggctcctgcctggcctctagaaagacaagctttatgatggccctgtgcctgt | |
| IL12A (p35 | ctagcatctacgaggacctgaagatgtatcaggtggagttcaagaccatgaacgccaagct | |
| subunit) | gctgatggaccccaagaggcagatctttctggatcagaatatgctggccgtgatcgacgagc | |
| (exemplary | tgatgcaggccctgaacttcaatagcgagacagtgcctcagaagtcctctctggaggagcc | |
| codon-optimized | agatttctacaagaccaagatcaagctgtgcatcctgctgcacgcctttcggatcagagccgt | |
| nucleic acid | gacaatcgaccgcgtgatgtcctatctgaatgcttcctaa | |
| sequence | | |
| encoding SEQ | | |
| ID NO: 41) | | |
| Version 3: non- | agaaacctccccgtggccactccagacccaggaatgttcccatgccttcaccactcccaaaa | 65 |
| codon-optimized | cctgctgagggccgtcagcaacatgctccagaaggccagacaaactctagaattttaccctt | |
| nucleic acid | gcacttctgaagagattgatcatgaagatatcacaaaagataaaaccagcacagtggaggc | |
| sequence | ctgtttaccattggaattaaccaagaatgagagttgcctaaattccagagagacctctttcataa | |
| encoding human | ctaatgggagttgcctggcctccagaaagacctcttttatgatggccctgtgccttagtagtatt | |
| IL12A (p35 | tatgaagacttgaagatgtaccaggtggagttcaagaccatgaatgcaaagcttctgatggat | |
| subunit) | cctaagaggcagatctttctagatcaaaacatgctggcagttattgatgagctgatgcaggcc | |
| (exemplary non- | ctgaatttcaacagtgagactgtgccacaaaaatcctcccttgaagaaccggatttttataaaa | |
| codon-optimized | ctaaaatcaagctctgcatacttcttcatgctttcagaattcgggcagtgactattgatagagtg | |
| nucleic acid | atgagctatctgaatgcttcctaa | |
| sequence | | |
| encoding SEQ | | |
| ID NO: 41) | | |

TABLE 5
Exemplary linker sequences (amino acid)

| | | SEQ ID |
|---|---|---|
| Name | Sequence | NO. |
| GS Linker | GGGGGGS | 24 |
| Elastin-like | VPGXG, wherein X is any amino | 44 |
| polypeptide | acid except proline | |
| linker | | |
| Elastin-like | VPGX₁GVPGX₂G, wherein | 45 |
| | X₁ is any amino | |
| polypeptide | acid except proline | |
| linker | and X₂ is any amino | |
| (multimerized) | acid except proline | |
| G₄S linker | GGGGS | 46 |
| (G₄S)₂ linker | GGGGSGGGGS | 47 |
| (G₄S)₃ linker | GGGGSGGGGSGGGGS | 48 |
| (G₄S)₄ linker | GGGGSGGGGSGGGGSGGGGS | 49 |

TABLE 6
Exemplary linker sequences (nucleic acid)

| | | SEQ ID |
|---|---|---|
| Name | Sequence | NO. |
| Exemplary | GGTGGCGGTGGCGGCGGATCT | 28 |
| nucleic acid | | |
| sequence | | |
| encoding GS | | |
| Linker of SEQ | | |
| ID NO: 24 | | |

TABLE 7
Exemplary IL-12 transgene sequences (amino acid)

| | | SEQ ID |
|---|---|---|
| Name | Sequence | NO. |
| Version 1: | MGHQQLVISWFSLVFLASPLVAIWELKKDVYVVELDWY | 22 |
| Human IL12 | PDAPGEMVVLTCDTPEEDGITWTLDQSSEVLGSGKTLTIQ | |
| transgene | VKEFGDAGQYTCHKGGEVLSHSLLLLHKKEDGIWSTDILK | |
| (Signal peptide- | DQKEPKNKTFLRCEAKNYSGRFTCWWLTTISTDLTFSVKS | |
| IL12B (P40 | SRGSSDPQGVTCGAATLSAERVRGDNKEYEYSVECQEDS | |
| Subunit)-GS | ACPAAEESLPIEVMVDAVHKLKYENYTSSFFIRDIIKPDPPK | |
| Linker-IL12A | NLQLKPLKNSRQVEVSWEYPDTWSTPHSYFSLTFCVQVQ | |
| (P35 Subunit)) | GKSKREKKDRVFTDKTSATVICRKNASISVRAQDRYYSSS | |
| | WSEWASVPCSGGGGGGSRNLPVATPDPGMFPCLHHSQNL | |
| | LRAVSNMLQKARQTLEFYPCTSEEIDHEDITKDKTSTVEACLP | |
| | LELTKNESCLNSRETSFITNGSCLASRKTSFMMALCLSSIYEDS | |
| | KMYQVEFKTMNAKLLMDPKRQIFLDQNMLAVIDELMQALNF | |
| | NSETVPQKSSLEEPDFYKTKIKLCILLHAFRIRAVTIDRVMSYLN | |
| | AS | |
| Version 1: | IWELKKDVYVVELDWYPDAPGEMVVLTCDTPEEDGITWT | 39 |
| Human IL12 | LDQSSEVLGSGKTLTIQVKEFGDAGQYTCHKGGEVLSHSL | |
| transgene | LLLHKKEDGIWSTDILKDQKEPKNKTFLRCEAKNYSGRFT | |
| (IL12B (P40 | CWWLTTISTDLTFSVKSSRGSSDPQGVTCGAATLSAERVR | |
| Subunit)-GS | GDNKEYEYSVECQEDSACPAAEESLPIEVMVDAVHKLKY | |
| Linker-IL12A | ENYTSSFFIRDIIKPDPPKNLQLKPLKNSRQVEVSWEYPDT | |
| (P35 Subunit)) | WSTPHSYFSLTFCVQVQGKSKREKKDRVFTDKTSATVICR | |
| | KNASISVRAQDRYYSSSWSEWASVPCSGGGGGGSRNLPV | |
| | ATPDPGMFPCLHHSQNLLRAVSNMLQKARQTLEFYPCTSEEI | |
| | DHEDITKDKTSTVEACLPLELTKNESCLNSRETSFITNGSCLAS | |
| | RKTSFMMALCLSSIYEDSKMYQVEFKTMNAKLLMDPKRQIFL | |
| | DQNMLAVIDELMQALNFNSETVPQKSSLEEPDFYKTKIKLCIL | |
| | LHAFRIRAVTIDRVMSYLNAS | |
| Version | MCHQQLVISWFSLVFLASPLVAIWELKKDVYVVELDWY | 42 |
| 2.1/Version 3: | PDAPGEMVVLTCDTPEEDGITWTLDQSSEVLGSGKTLTIQ | |
| Human IL12 | VKEFGDAGQYTCHKGGEVLSHSLLLLHKKEDGIWSTDILK | |
| transgene | DQKEPKNKTFLRCEAKNYSGRFTCWWLTTISTDLTFSVKS | |
| (Signal peptide- | SRGSSDPQGVTCGAATLSAERVRGDNKEYEYSVECQEDS | |
| IL12B (P40 | ACPAAEESLPIEVMVDAVHKLKYENYTSSFFIRDIIKPDPPK | |
| Subunit)-GS | NLQLKPLKNSRQVEVSWEYPDTWSTPHSYFSLTFCVQVQ | |
| Linker-IL12A | GKSKREKKDRVFTDKTSATVICRKNASISVRAQDRYYSSS | |
| (P35 Subunit)) | WSEWASVPCSGGGGGGSRNLPVATPDPGMFPCLHHSQNL | |
| | LRAVSNMLQKARQTLEFYPCTSEEIDHEDITKDKTSTVEACLP | |
| | LELTKNESCLNSRETSFITNGSCLASRKTSFMMALCLSSIYEDL | |
| | KMYQVEFKTMNAKLLMDPKRQIFLDQNMLAVIDELMQALNF | |
| | NSETVPQKSSLEEPDFYKTKIKLCILLHAFRIRAVTIDRVMSYLN | |
| | AS | |
| Version | IWELKKDVYVVELDWYPDAPGEMVVLTCDTPEEDGITWT | 43 |
| 2.1/Version 3: | LDQSSEVLGSGKTLTIQVKEFGDAGQYTCHKGGEVLSHSL | |
| Human IL12 | LLLHKKEDGIWSTDILKDQKEPKNKTFLRCEAKNYSGRFT | |
| transgene | CWWLTTISTDLTFSVKSSRGSSDPQGVTCGAATLSAERVR | |
| (IL12B (P40 | GDNKEYEYSVECQEDSACPAAEESLPIEVMVDAVHKLKY | |
| Subunit)-GS | ENYTSSFFIRDIIKPDPPKNLQLKPLKNSRQVEVSWEYPDT | |
| Linker-IL12A | WSTPHSYFSLTFCVQVQGKSKREKKDRVFTDKTSATVICR | |
| (P35 Subunit)) | KNASISVRAQDRYYSSSWSEWASVPCSGGGGGGSRNLPV | |
| | ATPDPGMFPCLHHSQNLLRAVSNMLQKARQTLEFYPCTSEEI | |
| | DHEDITKDKTSTVEACLPLELTKNESCLNSRETSFITNGSCLAS | |
| | RKTSFMMALCLSSIYEDLKMYQVEFKTMNAKLLMDPKRQIFL | |
| | DQNMLAVIDELMQALNFNSETVPQKSSLEEPDFYKTKIKLCIL | |
| | LHAFRIRAVTIDRVMSYLNAS | |

TABLE 8
Exemplary IL-12 transgene sequences (nucleic acid)

| | | SEQ ID |
|---|---|---|
| Name | Sequence | NO. |
| Version 1: | atgggtcaccagcagttggtcatctcttggttttccctggtttttctggcatctcccctcgtggcc | 26 |
| nucleic acid | atatgggaactgaagaaagatgtttatgtcgtagaattggattggtatccggatgcccctgga | |
| sequence of | gaaatggtggtcctcacctgtgacacccctgaagaagatggtatcacctggaccttggacca | |
| human IL12 | gagcagtgaggtcttaggctctggcaaaaccctgaccatccaagtcaaagagtttggagatg | |
| transgene | ctggccagtacacctgtcacaaaggaggcgaggttctaagccattcgctcctgctgcttcac | |
| (Human IL12 | aaaaaggaagatggaatttggtccactgatattttaaaggaccagaaagaacccaaaaataa | |
| transgene | gacctttctaagatgcgaggccaagaattattctggacgtttcacctgctggtggctgacgac | |
| (Signal peptide- | aatcagtactgatttgacattcagtgtcaaaagcagcagaggctcttctgacccccaaggggt | |
| IL12B (P40 | gacgtgcggagctgctacactctctgcagagagagtcagaggggacaacaaggagtatga | |
| Subunit)-GS | gtactcagtggagtgccaggaggacagtgcctgcccagctgctgaggagagtctgcccatt | |
| Linker-IL12A | gaggtcatggtggatgccgttcacaagctcaagtatgaaaactacaccagcagcttcttcatc | |
| (P35 Subunit)) | agggacatcatcaaacctgacccacccaagaacttgcagctgaagccattaaagaattctcg | |
| (exemplary | gcaggtggaggtcagctgggagtaccctgacacctggagtactccacattcctacttctccct | |
| nucleic acid | gacattctgcgttcaggtccagggcaagagcaagagagaaaagaaagatagagtcttcacg | |
| sequence | gacaagacctcagccacggtcatctgccgcaaaaatgccagcattagcgtgcgggcccag | |
| encoding SEQ | gaccgctactatagctcatcttggagcgaatgggcatctgtgccctgcagtggtggcggtgg | |
| ID NO: 22) | cggcggatctagaaacctccccgtggccactccagacccaggaatgttcccatgccttca | |
| | ccactcccaaaacctgctgagggccgtcagcaacatgctccagaaggccagacaaact | |
| | ctagaattttacccttgcacttctgaagagattgatcatgaagatatcacaaaagataaaa | |
| | ccagcacagtggaggcctgtttaccattggaattaaccaagaatgagagttgcctaaattc | |
| | cagagagacctctttcataactaatgggagttgcctggcctccagaaagacctcttttatga | |
| | tggccctgtgccttagtagtatttatgaagactcgaagatgtaccaggtggagttcaagac | |
| | catgaatgcaaagcttctgatggatcctaagaggcagatctttctagatcaaaacatgctg | |
| | gcagttattgatgagctgatgcaggccctgaatttcaacagtgagactgtgccacaaaaa | |
| | tcctcccttgaagaaccggatttttataaaactaaaatcaagctctgcatacttcttcatgctt | |
| | tcagaattcgggcagtgactattgatagagtgatgagctatctgaatgcttcctaa | |
| Version 1: | atatgggaactgaagaaagatgtttatgtcgtagaattggattggtatccggatgcccctgga | 61 |
| nucleic acid | gaaatggtggtcctcacctgtgacacccctgaagaagatggtatcacctggaccttggacca | |
| sequence of | gagcagtgaggtcttaggctctggcaaaaccctgaccatccaagtcaaagagtttggagatg | |
| human IL12 | ctggccagtacacctgtcacaaaggaggcgaggttctaagccattcgctcctgctgcttcac | |
| transgene | aaaaaggaagatggaatttggtccactgatattttaaaggaccagaaagaacccaaaaataa | |
| (IL12B (P40 | gacctttctaagatgcgaggccaagaattattctggacgtttcacctgctggtggctgacgac | |
| Subunit)-GS | aatcagtactgatttgacattcagtgtcaaaagcagcagaggctcttctgacccccaaggggt | |
| Linker-IL12A | gacgtgcggagctgctacactctctgcagagagagtcagaggggacaacaaggagtatga | |
| (P35 Subunit)) | gtactcagtggagtgccaggaggacagtgcctgcccagctgctgaggagagtctgcccatt | |
| (exemplary | gaggtcatggtggatgccgttcacaagctcaagtatgaaaactacaccagcagcttcttcatc | |
| nucleic acid | agggacatcatcaaacctgacccacccaagaacttgcagctgaagccattaaagaattctcg | |
| sequence | gcaggtggaggtcagctgggagtaccctgacacctggagtactccacattcctacttctccct | |
| encoding SEQ | gacattctgcgttcaggtccagggcaagagcaagagagaaaagaaagatagagtcttcacg | |
| ID NO: 39) | gacaagacctcagccacggtcatctgccgcaaaaatgccagcattagcgtgcgggcccag | |
| | gaccgctactatagctcatcttggagcgaatgggcatctgtgccctgcagtggtggcggtgg | |
| | cggcggatctagaaacctccccgtggccactccagacccaggaatgttcccatgccttca | |
| | ccactcccaaaacctgctgagggccgtcagcaacatgctccagaaggccagacaaact | |
| | ctagaattttacccttgcacttctgaagagattgatcatgaagatatcacaaaagataaaa | |
| | ccagcacagtggaggcctgtttaccattggaattaaccaagaatgagagttgcctaaattc | |
| | cagagagacctctttcataactaatgggagttgcctggcctccagaaagacctcttttatga | |
| | tggccctgtgccttagtagtatttatgaagactcgaagatgtaccaggtggagttcaagac | |
| | catgaatgcaaagcttctgatggatcctaagaggcagatctttctagatcaaaacatgctg | |
| | gcagttattgatgagctgatgcaggccctgaatttcaacagtgagactgtgccacaaaaa | |
| | tcctcccttgaagaaccggatttttataaaactaaaatcaagctctgcatacttcttcatgctt | |
| | tcagaattcgggcagtgactattgatagagtgatgagctatctgaatgcttcctaa | |
| Version 2.1: | atgtgccatcagcagctggtcatctcatggttctccctggtgtttctggcctcacctctggtcgc | 53 |
| codon-optimized | aatctgggaactgaaaaaggatgtgtacgtggtggagctggactggtatcccgatgcccctg | |
| nucleic acid | gcgagatggtggtgctgacctgcgacacacccgaggaggatggcatcacctggacactgg | |
| sequence of | atcagagctccgaggtgctgggaagcggcaagaccctgacaatccaggtgaaggagttcg | |
| human IL12 | gcgacgccggccagtacacctgtcacaagggaggagaggtgctgagccactccctgctgc | |
| transgene | tgctgcacaagaaggaggatggcatctggtccacagacatcctgaaggatcagaaggagc | |
| (Signal peptide- | caaagaacaagaccttcctgcggtgcgaggccaagaattatagcggccggttcacctgttg | |
| IL12B (P40 | gtggctgaccacaatctccaccgatctgacattttctgtgaagtctagcaggggatcctctgac | |
| Subunit)-GS | ccacagggagtgacatgcggagcagccaccctgagcgccgagagggtgcgcggcgata | |
| Linker-IL12A | acaaggagtacgagtattccgtggagtgccaggaggactctgcctgtccagcagcagagg | |
| (P35 Subunit)) | agtccctgcctatcgaagtgatggtggatgccgtgcacaagctgaagtacgagaattatacc | |
| (exemplary | agctccttctttatccgggacatcatcaagcccgatccccctaagaacctgcagctgaagcct | |
| codon-optimized | ctgaagaatagcagacaggtggaggtgtcctgggagtaccctgacacctggagcacacca | |
| nucleic acid | cactcctatttctctctgaccttttgcgtgcaggtgcagggcaagtccaagcgggagaagaa | |
| sequence | ggacagagtgttcaccgataagacatctgccaccgtgatctgtagaaagaacgcctctatca | |
| encoding SEQ | gcgtgagggcccaggaccgctactattctagctcctggtccgagtgggcctctgtgccttgc | |
| ID NO: 42) | agcggcggaggaggaggaggatctaggaatctgccagtggcaacccctgacccaggc | |
| | atgttcccctgcctgcaccacagccagaacctgctgagggccgtgtccaatatgctgcag | |
| | aaggcccgccagacactggagttttacccttgtaccagcgaggagatcgaccacgagg | |
| | acatcacaaaggataagacctccacagtggaggcctgcctgccactggagctgaccaa | |
| | gaacgagtcctgtctgaacagccgggagacaagcttcatcaccaacggctcctgcctgg | |
| | cctctagaaagacaagctttatgatggccctgtgcctgtctagcatctacgaggacctgaa | |
| | gatgtatcaggtggagttcaagaccatgaacgccaagctgctgatggaccccaagagg | |
| | cagatctttctggatcagaatatgctggccgtgatcgacgagctgatgcaggccctgaact | |
| | tcaatagcgagacagtgcctcagaagtcctctctggaggagccagatttctacaagacca | |
| | agatcaagctgtgcatcctgctgcacgcctttcggatcagagccgtgacaatcgaccgcg | |
| | tgatgtcctatctgaatgcttcctaa | |
| Version 2.1: | atctgggaactgaaaaaggatgtgtacgtggtggagctggactggtatcccgatgcccctgg | 63 |
| codon-optimized | cgagatggtggtgctgacctgcgacacacccgaggaggatggcatcacctggacactgga | |
| nucleic acid | tcagagctccgaggtgctgggaagcggcaagaccctgacaatccaggtgaaggagttcgg | |
| sequence of | cgacgccggccagtacacctgtcacaagggaggagaggtgctgagccactccctgctgct | |
| human IL12 | gctgcacaagaaggaggatggcatctggtccacagacatcctgaaggatcagaaggagcc | |
| transgene | aaagaacaagaccttcctgcggtgcgaggccaagaattatagcggccggttcacctgttggt | |
| (IL12B (P40 | ggctgaccacaatctccaccgatctgacattttctgtgaagtctagcaggggatcctctgacc | |
| Subunit)-GS | cacagggagtgacatgcggagcagccaccctgagcgccgagagggtgcgcggcgataa | |
| Linker-IL12A | caaggagtacgagtattccgtggagtgccaggaggactctgcctgtccagcagcagagga | |
| (P35 Subunit)) | gtccctgcctatcgaagtgatggtggatgccgtgcacaagctgaagtacgagaattatacca | |
| (exemplary | gctccttctttatccgggacatcatcaagcccgatccccctaagaacctgcagctgaagcctc | |
| codon-optimized | tgaagaatagcagacaggtggaggtgtcctgggagtaccctgacacctggagcacaccac | |
| nucleic acid | actcctatttctctctgaccttttgcgtgcaggtgcagggcaagtccaagcgggagaagaag | |
| sequence | gacagagtgttcaccgataagacatctgccaccgtgatctgtagaaagaacgcctctatcag | |
| encoding SEQ | cgtgagggcccaggaccgctactattctagctcctggtccgagtgggcctctgtgccttgca | |
| ID NO: 43) | gcggcggaggaggaggaggatctaggaatctgccagtggcaacccctgacccaggca | |
| | tgttcccctgcctgcaccacagccagaacctgctgagggccgtgtccaatatgctgcaga | |
| | aggcccgccagacactggagttttacccttgtaccagcgaggagatcgaccacgagga | |
| | catcacaaaggataagacctccacagtggaggcctgcctgccactggagctgaccaag | |
| | aacgagtcctgtctgaacagccgggagacaagcttcatcaccaacggctcctgcctggc | |
| | ctctagaaagacaagctttatgatggccctgtgcctgtctagcatctacgaggacctgaag | |
| | atgtatcaggtggagttcaagaccatgaacgccaagctgctgatggaccccaagaggc | |
| | agatctttctggatcagaatatgctggccgtgatcgacgagctgatgcaggccctgaactt | |
| | caatagcgagacagtgcctcagaagtcctctctggaggagccagatttctacaagacca | |
| | agatcaagctgtgcatcctgctgcacgcctttcggatcagagccgtgacaatcgaccgcg | |
| | tgatgtcctatctgaatgcttcctaa | |
| Version 3: non- | atgtgtcaccagcagttggtcatctcttggttttccctggtttttctggcatctcccctcgtggcca | 66 |
| codon-optimized | tatgggaactgaagaaagatgtttatgtcgtagaattggattggtatccggatgcccctggag | |
| nucleic acid | aaatggtggtcctcacctgtgacacccctgaagaagatggtatcacctggaccttggaccag | |
| sequence of | agcagtgaggtcttaggctctggcaaaaccctgaccatccaagtcaaagagtttggagatgc | |
| human IL12 | tggccagtacacctgtcacaaaggaggcgaggttctaagccattcgctcctgctgcttcaca | |
| transgene | aaaaggaagatggaatttggtccactgatattttaaaggaccagaaagaacccaaaaataag | |
| (Signal peptide- | acctttctaagatgcgaggccaagaattattctggacgtttcacctgctggtggctgacgaca | |
| IL12B (P40 | atcagtactgatttgacattcagtgtcaaaagcagcagaggctcttctgacccccaaggggtg | |
| Subunit)-GS | acgtgcggagctgctacactctctgcagagagagtcagaggggacaacaaggagtatgag | |
| Linker-IL12A | tactcagtggagtgccaggaggacagtgcctgcccagctgctgaggagagtctgcccattg | |
| (P35 Subunit)) | aggtcatggtggatgccgttcacaagctcaagtatgaaaactacaccagcagcttcttcatca | |
| (exemplary non- | gggacatcatcaaacctgacccacccaagaacttgcagctgaagccattaaagaattctcgg | |
| codon-optimized | caggtggaggtcagctgggagtaccctgacacctggagtactccacattcctacttctccctg | |
| nucleic acid | acattctgcgttcaggtccagggcaagagcaagagagaaaagaaagatagagtcttcacgg | |
| sequence | acaagacctcagccacggtcatctgccgcaaaaatgccagcattagcgtgcgggcccagg | |
| encoding SEQ | accgctactatagctcatcttggagcgaatgggcatctgtgccctgcagtggtggcggtggc | |
| ID NO: 42) | ggcggatctagaaacctccccgtggccactccagacccaggaatgttcccatgccttcacc | |
| | actcccaaaacctgctgagggccgtcagcaacatgctccagaaggccagacaaactctag | |
| | aattttacccttgcacttctgaagagattgatcatgaagatatcacaaaagataaaaccagcac | |
| | agtggaggcctgtttaccattggaattaaccaagaatgagagttgcctaaattccagagagac | |
| | ctctttcataactaatgggagttgcctggcctccagaaagacctcttttatgatggccctgtgcc | |
| | ttagtagtatttatgaagacttgaagatgtaccaggtggagttcaagaccatgaatgcaaagct | |
| | tctgatggatcctaagaggcagatctttctagatcaaaacatgctggcagttattgatgagctg | |
| | atgcaggccctgaatttcaacagtgagactgtgccacaaaaatcctcccttgaagaaccgga | |
| | tttttataaaactaaaatcaagctctgcatacttcttcatgctttcagaattcgggcagtgactatt | |
| | gatagagtgatgagctatctgaatgcttcctaa | |
| Version 3: non- | atatgggaactgaagaaagatgtttatgtcgtagaattggattggtatccggatgcccctgga | 68 |
| codon-optimized | gaaatggtggtcctcacctgtgacacccctgaagaagatggtatcacctggaccttggacca | |
| nucleic acid | gagcagtgaggtcttaggctctggcaaaaccctgaccatccaagtcaaagagtttggagatg | |
| sequence of | ctggccagtacacctgtcacaaaggaggcgaggttctaagccattcgctcctgctgcttcac | |
| human IL12 | aaaaaggaagatggaatttggtccactgatattttaaaggaccagaaagaacccaaaaataa | |
| transgene | gacctttctaagatgcgaggccaagaattattctggacgtttcacctgctggtggctgacgac | |
| (IL12B (P40 | aatcagtactgatttgacattcagtgtcaaaagcagcagaggctcttctgacccccaaggggt | |
| Subunit)-GS | gacgtgcggagctgctacactctctgcagagagagtcagaggggacaacaaggagtatga | |
| Linker-IL12A | gtactcagtggagtgccaggaggacagtgcctgcccagctgctgaggagagtctgcccatt | |
| (P35 Subunit)) | gaggtcatggtggatgccgttcacaagctcaagtatgaaaactacaccagcagcttcttcatc | |
| (exemplary non- | agggacatcatcaaacctgacccacccaagaacttgcagctgaagccattaaagaattctcg | |
| codon-optimized | gcaggtggaggtcagctgggagtaccctgacacctggagtactccacattcctacttctccct | |
| nucleic acid | gacattctgcgttcaggtccagggcaagagcaagagagaaaagaaagatagagtcttcacg | |
| sequence | gacaagacctcagccacggtcatctgccgcaaaaatgccagcattagcgtgcgggcccag | |
| encoding SEQ | gaccgctactatagctcatcttggagcgaatgggcatctgtgccctgcagtggtggcggtgg | |
| ID NO: 43) | cggcggatctagaaacctccccgtggccactccagacccaggaatgttcccatgccttcacc | |
| | actcccaaaacctgctgagggccgtcagcaacatgctccagaaggccagacaaactctag | |
| | aattttacccttgcacttctgaagagattgatcatgaagatatcacaaaagataaaaccagcac | |
| | agtggaggcctgtttaccattggaattaaccaagaatgagagttgcctaaattccagagagac | |
| | ctctttcataactaatgggagttgcctggcctccagaaagacctcttttatgatggccctgtgcc | |
| | ttagtagtatttatgaagacttgaagatgtaccaggtggagttcaagaccatgaatgcaaagct | |
| | tctgatggatcctaagaggcagatctttctagatcaaaacatgctggcagttattgatgagctg | |
| | atgcaggccctgaatttcaacagtgagactgtgccacaaaaatcctcccttgaagaaccgga | |
| | tttttataaaactaaaatcaagctctgcatacttcttcatgctttcagaattcgggcagtgactatt | |
| | gatagagtgatgagctatctgaatgcttcctaa | |
| Nucleic acid | atgtctgcacttctgatcctagctcttgttggagctgcagttgctgactacaaagacgatgacg | 30 |
| sequence of | acaagctttgggagctggagaaagacgtttatgttgtagaggtggactggactcccgatgcc | |
| mouse IL12 | cctggagaaacagtgaacctcacctgtgacacgcctgaagaagatgacatcacctggacct | |
| transgene | cagaccagagacatggagtcataggctctggaaagaccctgaccatcactgtcaaagagttt | |
| | ctggatgctggccagtacacctgccacaaaggaggcgagactctgagccactcacatctgc | |
| | tgctccacaagaaggaaaatggaatttggtccactgaaattttaaaaaatttcaaaaacaaga | |
| | ctttcctgaagtgtgaagcaccaaattactccggacggttcacgtgctcatggctggtgcaaa | |
| | gaaacatggacttgaagttcaacatcaagagcagtagcagttcccctgactctcgggcagtg | |
| | acatgtggaatggcgtctctgtctgcagagaaggtcacactggaccaaagggactatgaga | |
| | agtattcagtgtcctgccaggaggatgtcacctgcccaactgctgaggagaccctgcccatt | |
| | gaactggcgttggaagcacggcagcagaataaatatgagaactacagcaccagcttcttcat | |
| | cagggacatcatcaaaccagacccgcccaagaacttgcagatgaagcctttgaagaactca | |
| | caggtggaggtcagctgggagtaccctgactcctggagcactccccattcctacttctccctc | |
| | aagttctttgttcgaatccagcgcaagaaagaaaagatgaaggagacagaggaggggtgta | |
| | accagaaaggtgcgttcctcgtagagaagacatctaccgaagtccaatgcaaaggcggga | |
| | atgtctgcgtgcaagctcaggatcgctattacaattcctcgtgcagcaagtgggcatgtgttcc | |
| | ctgcagggtccgatcctctagaggtagtggatccggtggcagtggaggttctggatctggta | |
| | agcttagggtcattccagtctctggacctgccaggtgtcttagccagtcccgaaacctgctga | |
| | agaccacagatgacatggtgaagacggccagagaaaaactgaaacattattcctgcactgc | |
| | tgaagacatcgatcatgaagacatcacacgggaccaaaccagcacattgaagacctgtttac | |
| | cactggaactacacaagaacgagagttgcctggctactagagagacttcttccacaacaaga | |
| | gggagctgcctgcccccacagaagacgtctttgatgatgaccctgtgccttggtagcatctat | |
| | gaggacttgaagatgtaccagacagagttccaggccatcaacgcagcacttcagaatcaca | |
| | accatcagcagatcattctagacaagggcatgctggtggccatcgatgagctgatgcagtct | |
| | ctgaatcataatggcgagactctgcgccagaaacctcctgtgggagaagcagacccttaca | |
| | gagtgaaaatgaagctctgcatcctgcttcacgccttcagcacccgcgtcgtgaccatcaac | |
| | agggtgatgggctatctgagctccgcctaa | |

TABLE 9
Exemplary chimeric NDV genomic RNA sequences

| | | SEQ ID |
|---|---|---|
| Name | Sequence | NO. |
| NDV La Sota | accaaacaaagauuuggugaaugacgagacuacacucaagaauaauugugcgcaac | 50 |
| genomic RNA | cuuuuuuuaagacauuuauuugaguucgaauucgaguccuaaggagucaggguuc | |
| | aacuuuuuucuaacauaauaugauuaaaucaacaagaauacaauuggccaaaaaag | |
| | gagccuauuaauaugugauuuucguuaagagucacaguuacuguaauauccuuug | |
| | acugcauugccuauaguuuucauguagaauuuuuguugagcacgagucaaguaca | |
| | guacagaagugucuguaaacauuucuuugaguuugguaauaccuaggacagccuu | |
| | caaauauuuagggcagguacuaugcuucaaguauguccuuagugggauaaggucc | |
| | uccauggagaucaugccuuuaagcacuaggcugauuauaucgccuauuuuauuga | |
| | gauuuucgacucuaagaccuaguauuguaaccucuaggaucugucucgugcacug | |
| | uuuaagugauguccucuuuuucccgucaguagagagauuguaagggguaaauaga | |
| | aauacugugucagcgagaucacccucagcuuccauauauaucacagaccggauaac | |
| | ugugucaaugugacuagcgaugaucuggguuaucugaguuauguucgcuagcgu | |
| | gcucaccagacucuccgcacagaauggacggacgggcugucccccggcuucaauca | |
| | gcgcagugucaauauucuuccucaaguacuuuauuaaucuugguaaaggacugga | |
| | uaggaugucugucacacgcugccgcugugaggugaauaaccuggucagugugauc | |
| | ucaucugauuuagacaaaagcguaccgugccgcugcaccagaguuuucgccauccu | |
| | caccaccucauguacaaauguaggcccgcccagguaacccaugacaaauaccaggua | |
| | acacuccauaucuccucgacaugcauaaccauuagagagaauauauccuuuugugg | |
| | aacacggagcaaacaaguucaugaguagaugaaaguaguaucccauugcauacaac | |
| | acuuugaugauuacuaccccgcccucccuuacagaaugcauggcaaucagagauaa | |
| | auugauagcuaguugaucuaguaagcuuugauuggacccuggaggaauuucaaug | |
| | ucacaaugcagcaaugauacagaucuguagggcacugcagauguaauauaccccac | |
| | uacuuuaucugaggucaggucgcuuuccucuguauuuucucuccauaauggacgg | |
| | aacucuuggacaaauccaucuuugcauguuaccuccgccuguagauuccuauaaac | |
| | aaccgaauucaaaaacugaguuggggucggcccgaaaugucguugcgggggguuc | |
| | aucucauuugaaaagagcguauuguaauagauaguuucaugugguacaugcaguu | |
| | cgagaagacucaugauggcuccgcucccuucagcuaaguauaaggaguucccgugu | |
| | cuugcacaucuuaccucggguacagaaaggagaugagaugccuuauaccaagagga | |
| | agaugcagucccuaucccucugaacaaguaucguacuaaguagucuuccuccggau | |
| | uuggagaugugaguucaggaugaaucugacuaagugugaaugcgucauaccuugc | |
| | uggagcacucaaaucuaacucuugcaaaaaugccgcagguugucgggugaaugga | |
| | ucuuucacucgagcaccaauaucuugcacagaagggaacucuaauaauggcucuug | |
| | ggggaacaacaacgccaggauaguaucccuguccucccuuucccugaucaaauuga | |
| | ggcucuuccgagacauguaguacagauuagcugggaauguaauuauguuaggaga | |
| | caugauagagcucacuugaucggggcuaaguaaugguuucacagcauccgacagua | |
| | aauacucagugaguauugaacauuucucuucugcaguuaagccucuuauuuucgg | |
| | gauuucucuuguuguagcaaagaguaccguguacagacagcauaaccgggauauca | |
| | gcugaagcaucuucucauucagguuaucaucuaagacagaugggaacagcagauca | |
| | uacuuauuuccugaauauaagccggagaucacacgucgggugcaagauacuaauag | |
| | uuuugcagacauuucgauaaaauccguaucugcaaguuggugugauccgucaugg | |
| | uugaccaggcccacugcauguaaccuugaaugaaugacgggaugagauauuguag | |
| | cugcaauguuggaaaguagaauuccuggcauauucuuguauaaaucacccauauau | |
| | aagacaauauuguccaggccucuuacucucagguaauagaguugguaagaacaguc | |
| | gaggagcacuucaagugcugcauauucaaauaggcggaccacaucugaauucugag | |
| | cuucacugauccaauuucggguauugucauacacuauuauggcgucauucuuuau | |
| | ggagguaucuucaucauaagaaaccacagacuggccaaucaacuucccgcuggaua | |
| | uugaaagaauguucauuagcucuaucgugggauaugacuccagauuaagcucaua | |
| | acucuugaagauagcuaagucaagucucgcaaagucucccuccgauacagggcuag | |
| | gaucauacauaaacuuauuugaggucacuguccucaguuccgguaccaccccaagu | |
| | agcucgaaaggaaccgcaacaggugcuucucugauacagcaacuaaauuuacuaug | |
| | uaggugcagugugaucucaucauauguccugguuguugucauuggaaagaucgau | |
| | ucgauuagagauaaacccaagagcaugaucuguugguaaaccacauuccccucuuu | |
| | gacuccuucuucagugaacagccuuugagaaucauuggauaugugaauguaaggu | |
| | gacacccuguagagagaugcaggggugaaugucaucugaguuauaccaucaucua | |
| | gucuauguugaagauucccagccguggguaaaggggacaguaaccgaagauacuc | |
| | uaaguuuacauuacaccgagauuuugcaaucguaagagcagcaguccaauuuacuu | |
| | cauuauccccauaagcccagaucaacacggaugaugcccuuagggcagccuuuaca | |
| | uguggcgacauaugagcuauuuuugcaagugaggcagcucuccucuccugugucu | |
| | uugacccgagauaugguacccucaucggaggauucuugcuggugucaucggucaa | |
| | uucuauauugcuuggaagauggaaccaaguaaauuguucaucuccgcugucacau | |
| | cuuguacacccuccgcuuacacuaagaaucucacccucuacgaguucuaucguauc | |
| | aggauuagauacacccaguauuuuccugccucccgucaaaggugaccagcuucuau | |
| | uccgugcauagucugccagugucagagaacacauauuagaagagacuaaggggug | |
| | guuggaucuacuggaggaaaaaacaucgucucuaaacagcauugcaugcaugcuag | |
| | aauaauugacuauccgcaucagccucuugaugccuaauggccuccuaguaagcgca | |
| | aucuuaauuacgguguuuguugugucaacaagcccuugaauuugcuuucuccuac | |
| | cuacagagcuugccuccaugauggcaugcgcaacgcggggaugaaucaccucuuga | |
| | uuaagcaagaauucagccaaugccuucucuucugccucauuauccucugugugcac | |
| | uccagacaauaagggauuugaacaaguuucaaauaggacucuuugcguauguuuc | |
| | uuaagaacaauauuugggcuugcaacagucucaaaauugaaagaguaugggucgu | |
| | ugcacagacuggcccaaucuccauucccaggcggccuaguuaagauauuagucaua | |
| | auguuaggacucaguaaucccacugcuucuagucgcuugaucucugcaaaagcagu | |
| | aguccccgggucaccgauauuucuaguguagagccuugaguauugaagguuacuc | |
| | agucccccuaauugggcaggagucagaacauaugagugcacaaaagagauguccuc | |
| | aauccacgacugauuaagaucggggugcgaauuguuggugauggagaacucagag | |
| | ucaaaguaugucugcacacaacucauuauauaguuuaaauaguaacagaagucuuu | |
| | gggaagcccguucucgcauagccgugcuacaguagaggcaauguuggcacaggaca | |
| | uuacgguguuuucacugagaucaccugacacuagcacuaauuuagaugaauuuuu | |
| | gaggacuugacugaggauugcuccaucuuugaagauucguuugcuguauaugaag | |
| | aaugugucugaccugaugguuucacgauccuucaaauuauggccaaucaaaugau | |
| | ugacaugaauuaauuccuugaagaaauuaucacuggcuugaugcaacugugucaac | |
| | accaucuccggagagucgucugaucuuaccucucucguuacugcuauuacuugau | |
| | uaucacccuguaccauacaggcaacacgacaaugcgaucuagcugcagcaaguugg | |
| | auugcagcaauugagaucauuguccauagcuucuggcauaauccuucgauaccccc | |
| | ucuggcacugacaauauauaugucaucauuagggacucuugagaggucacaguca | |
| | guagggucacuuggaggauugaaagggucuccuacgaacaucguaguguccauca | |
| | gucuuaggugaauccauucgaagaagugagguaggcccaucaacugauugauggc | |
| | augagcgaacaauuugauugucugauaucuccaauuaagacaguacuuuugcagg | |
| | ucaguuguuaugaagguugcaacucuccgacgguucuugcuuuucggaucaugau | |
| | ugcgguuugaagauacucuuucuuuacagucagugauacguuucuuauugcuguu | |
| | aaaagacaguugacucaucgcuagcauacucuuggucaaggauaugcuauccugaa | |
| | ugacuccauuucccugaaagaaaggugcaaucugaucggcuaggaucccuuccgcc | |
| | aucaccugacaguuccuuaacuucuuugucagcuuagcgaagauccguccauuaac | |
| | uuucacuuccuucuccuugagcgaguaugauacugccacauugucaucucuaagg | |
| | uacucaagggucgucagauauuccaucucuuuauauggaucaaaaucauuugacuc | |
| | uaaaaacucuaucaagaggcgauuagucgaaguugcuucuuuuacauguuucuuc | |
| | uggucuucggagagaagguuccgccuaaacgaggcaagccaauuaucguuggggu | |
| | gugcgauugccuugucuuuuaggaacaugcucagguuggugacagggucauauuc | |
| | uauacauggcucaaauucaagugcagauaaacucuuauacucucucaacaugauau | |
| | cgugugaaaucucugcugaaucugcauguaguugcccaaugaccuucccauauau | |
| | uguauccacuuugacucgcggccacacaccugcauucuucuuucuguacccguuga | |
| | ugauuguucccuugaagaaagacaguaccugaaggaucauaucaaagucuaccauu | |
| | uucggugcgcacauuuggcuccugacugccuuugcugcaauacgggacucaagca | |
| | gugggugaccccacagacgcaacagacacaacaucucagcugcuugauucuguucu | |
| | aaaccagagaauacaguagcgauugcaugagucacggauucugcuauaucauuggg | |
| | gaggaggccaauuagaaugucuuuaagcuccugcagguugaaugcgaagaaaucu | |
| | ccugcaaauguaccugacggcucgaguagcuggacagcuccguaugcaaaucccuc | |
| | cauuagugauacaacaucguagacuugauuacccaagucuuuugccagagcgucua | |
| | uuaaccgcaaaaugucaucaauuuucucugauaagcuucugagaugcaccgccgug | |
| | guugauauuauguugaccauaucucugcccuccaucauaucugcauacaucaauac | |
| | aaguuccuggguaagacaugugaacuuguucucauucguaugcgucacaacgacaa | |
| | guucaggagugacaaagacuuggccuaccuuauggguuagcaucaccaauuuguu | |
| | ggccgcagaccuuguccuagcugccaccaucagaugccucuggaucuguuuuaua | |
| | uggagccaugcaaacuuggcuguggaccauuuugagugaaaccagaaugccggau | |
| | ccguacgaaugcugcugaacuccucugaccgggggacauuguuagaccaagaugac | |
| | cccagcaguuucuucucuauaugcguacacagccuugugaacaguucuccauaucu | |
| | cguguugugaauuuggaucuucuucucaaucuuccgaaauuuguugguugaaucu | |
| | gggaccucaauauuagccaguucuucuaaacaccugggguggagcacuccgguua | |
| | uucuggaauugugguuaagaguuugguguacugcccuuccgaguuuuaucauuc | |
| | ucucaguaucaggagaggccgauucaaguauuuuuuuccauugucggcugagaau | |
| | gagguggucgaagucacauucaucaggaagcgguagcccaguuaauuuccaguaa | |
| | uagaguaguuugugcuugaccaauggugaagacaggugugacucugguaggauaa | |
| | ucugaugcucugcccuuucaggaccggagcucgccauguccuacccguauuguua | |
| | accaugagcuguuuugccuuguaucucauugccacuuacauuuuuucuuaaucaa | |
| | gagacuauugacaaggcuuaaucugaucgcaugagcacuggcugauuguggucaa | |
| | cuggcaacauggaauucgagcacgcgccggcauucgguuugauucuugaugucgc | |
| | agaagauaggugauacaaugccaucuuuccaacuccuuuauaauugacucaacuag | |
| | ccagaccuggcuucucuaaccccgucaucuuugaggaucucaacuaguaacgggac | |
| | gauucugaauucuccgaagagaguauuagauauuucagcaaugcugagacaauag | |
| | gucuuauuagucuugaccacuuuaaaacaaguugauguuguguaugcugcuuugg | |
| | uacugcuugaacucacucgaguaaugcgacugcgggaugugcuaucgaauacugc | |
| | agacgcaggguuaagucuugcuuguacaccaucaagcauugucccgaauaccccuc | |
| | gcaaggugugguuucuauagaagauuaggggauauggaucuguauagacuccagu | |
| | aacacacgaguuggggcaucuugcugaagccuggcaagggauacuaccuggccgag | |
| | ugaaggcauugaauguauaaggacuaugaagaguggcuguuuuguugcugacugu | |
| | cauaggauauaauaacgcgggagagaaguaugaugacccucguugauacaagaaau | |
| | gagaugucccuacugugagaauucugccuucggcccccaugagugugacuguguu | |
| | gggcgguacagucaguaccgggucuucgccuaaggauguugacaccuugauagau | |
| | aagauagccugcuguaugcguuucccaccaaaccguccaggcuuauacgaagacuu | |
| | ggccauucgaaucugguagucuugcucaucugggcaugugucauuguaucgcuug | |
| | uauaucacauauuucccuuccuguacagugucacugggugaauuggguuuuaacc | |
| | cuccguagacugagaaccauacgcggcugucaauaaaagauccacccccuacuccu | |
| | ggguaguuggccacccaguccccgaauaauguugugacaucuagguccuuuucgu | |
| | gguacuggccgucgaacccuaaccucccauguaccauccgcguagggacagcugag | |
| | uuauaaucuucuuccucugucuccgugacuuucgagcacagcauaucacaacccag | |
| | gggaguugcacucacacugcaagacuuccgauuuugggugucguccagguugaug | |
| | gaacgcagaguagaaaagaauacccucccuguugcagauguccggagcacaccaag | |
| | ugcuaaauacugauaugaaugugagugaucucugcauccagacaauauuacauuau | |
| | ggguguagcaguaauggguagcacucaugucaaaugaggguauucgagugcaacc | |
| | ugauccuguaguaggcgccgggauaaaauucagauguucuugaaaugcagaggga | |
| | uagaaugaugugacaucacuagcaucaucuacaaugaguucuuugccuaucccccc | |
| | uauauaaucugggucauggauaggugccccccacccacuguuguuugcagcucca | |
| | uuaaucugauaagagagagauguuauugcguucauaauuguggucucaguauuua | |
| | acaaugccaacggagacucaagggccacuugcuuauauauccuaucuacuacaucu | |
| | ugauuggaaccaaguguagauguaaucuuuucuucugcccuggaaauccuagucg | |
| | guaugccuacaagaucgcuaggugugcuagcccccaugcuauauaaaagggaggcu | |
| | acagauauagccaaggucacuacuguuaagaauaagauugcaauccggaauaucaa | |
| | gcgccauguauuuuuugccucucuuucaucauucucuaacgcaacuuggcuaacg | |
| | gcgcgguccaugauugaggacuguugucggugaagcgguagaacggagguuguga | |
| | agccuggcucgcaauugaggggcggccucucuuaccguucuacccguauaucguc | |
| | cuuuggucaucuacaaccgguaguuuuuucuuaacucucugaacugacagacuacc | |
| | agaacuuucacacaaauuacuauuagggaaaccuucguuccucaucuguguucaca | |
| | uuuuuguaguggcucucaucugaucuagaguauuauucccaagccauaauaaggu | |
| | cuuuuguugcgccuuuugcuuguacauuagguagcaugcuagaaucaggcuaagu | |
| | auaccaaaaacaagagauaugauagucaaaacgauauagguaaugagagcagaugu | |
| | gcuagucaguuugacauugacuuugucuaguuuucuguugcuuuccucuaacuua | |
| | uucaaagcauuacugaucgaguuguugacauucccaagcucaguugagauaucaag | |
| | auugccuguuauuauuacuugagaaucuuguauugagauauucuucugauaaguu | |
| | acaucgaauuccccacugagccuuaaaguuaucccgccuaaggauaaaacauugca | |
| | ugauuguuuaucuauuagagacacggcuucuccauaguuuugcgauaugauaccc | |
| | gggggguuuacacaucuacauguugucaucuugcaguuggcgaugacugaaccuu | |
| | ugauagucauguaugguguaguaagugcgccuucggucuuugaguacauacaggc | |
| | cgacguauugccgcucaagcaggaauaaauaccaggggacauagggaacguuacua | |
| | uucuuguacaauauaaaucuaagucaguuucuauacaguaugaggugucaaguuc | |
| | uucuaucacagaaccgaccugugucaccacuuuugggacaagugccgaggcaaauc | |
| | cccugguugugcuuacggauaagguuuccaaguagguggcacgcauauuauuuag | |
| | guucccgacugaaggugcaguuaccuguauacccaagaguugagucugugagucg | |
| | uauagaauaggguuaccggugauuaagccgcuaccgauuaaugagcugaguugau | |
| | uguucccuacaccuaacuuagucaauaaguaauccauauuuccaccagcuagauug | |
| | uaaagugccugaauagucagcuuguuuaaagcaggugaagugauuugugguccga | |
| | auacuguagucaauucgguuagguacagguugagcucuacaccaacuugcugugc | |
| | aauuuugaugcagucuaauuccugagcuguuuuauuaaauuggucauuaacaaac | |
| | ugcugcaucuucccaacugccacugcuaguugcgauaauccgucagugaccucaug | |
| | cacagccucauugguugcggcaaugcucucuuuaagucggaggauguuggcagca | |
| | uuuuguuuggcuuguaucagagcugcggccgcuguuauuugugcggcaguugca | |
| | accccaagagccacaccgccaauaauggcgccuauaaggcgccccugucuccccccu | |
| | ccagauguagucacagacucuuguauccuacggauagagucaccaagggggguga | |
| | gcaaaguggucaauguccuguuguaugcauccaagggggcuuucgcacaugccuc | |
| | cuuauccuugggcagauucgggaggagcuuaacuaugauugauccugucugggau | |
| | gagguguauauguugacggcuuugucuccuguaaccacaauuccugcagcugcaa | |
| | gaggccugccaucaauggaguuugccggacagaugcaacucaguaccagcgcaacc | |
| | cggauagucagcaucauaggugcuggguucuugguagaaggucuggagcccaucu | |
| | ugcaccuggagggcgccaaccgggauccagaaucuucuacccguguuuuuucuaac | |
| | uugauagacagguaaacuaacuguaaauaaucaagacagauuaguuuuuuguguc | |
| | augaugaucugggugagugggcggagcgcaaucucagagacgcagcuuauuucuu | |
| | aaaaggauuguauuuggcaagggugugccccuucuccagcuuaguagagguaacc | |
| | ucguggucggcggucacugcgacagcgcguuggguaccugcuuggauaaugauuu | |
| | uaacgcuccgcaggcacgcgguuugacuccagaguaucuuggccaccugaggagaa | |
| | gcauuugcuaugggauagcaggcugucccacugcuagagaagaaaggugccaaaag | |
| | cuuaguccgugcaccucuugcuuuuaccaacacggaaggcccgagcacaucacuga | |
| | gcccgacagauagaucaaggcuccuuauuuucuuuuccagcuugucaaaugucacu | |
| | uucuuccccuuccuaucuacaguggucauaaguccaauaugcaagaagagguuagc | |
| | auaguauccgcugucagacuuagacagagauuuaaccaaaggacuccucgggucua | |
| | ccuccacauuaauagugacauugagcgcaagauuguacagacucgagccagaaacc | |
| | uucaauacugcagcugggaucuuguagacaucccucuucgguaccacagucaagga | |
| | gacaaaguucaccuuguauucuaggguuccacucccgggaaucuucucuggcgcu | |
| | uucacgugcuugacugcauucacugaugaguauuuguuugccacaacccuacagc | |
| | uuugcagcacuuggggugccugcacuacugagaaaaccauucucucaguauuagu | |
| | ugcacucuucuugcaugugacuaucauagugagacaggcccuugccagcucaauaa | |
| | ggucuccgguauuugggacgcuuccuaggcagagcaucgcagcggaaaguaacuc | |
| | gcgcuuggguuuaucaucgaucaugccgacgguggcuucuucauucccaacuuga | |
| | aagaugaauccauagguggugaugaauacugaguccuccuuacuaucaguccacaa | |
| | gucaaggcgcuggauccuauauugcggggcgauuugcuucuucccaucuccugug | |
| | ucuuguaggacgaucggaaaugcuaacagguugcuagaagaaugggcagaaucaaa | |
| | guacagcccaauuguccuagaugaguccaucuuggcacaauuggggcacuccaauu | |
| | cuacccguauuuuuucuuaaucuuaaauguagcuagauuaauuacgguuacgcga | |
| | ucauucaguggggcugaggaagcgagagaggauugccgcuuggagaguuggaccu | |
| | uggguccgcgggggggaacucggugcagauuccgugugaugccgaguggacaggg | |
| | acccgcuacguguggcaguaguaauuagccauuuagagcaaggcgcuugauuuuc | |
| | cugauuuccucgaucgacccggcugcaucuaacuugcuuaggagcuuggcugaag | |
| | aacucgggugcauugggcgugacaugaucaaugcacggacaguguccuuuuccac | |
| | uccuauaucaggcccgcaugcaguggcggguuuaaucaauucagauggauguggc | |
| | acugguugcgaaaguuuauuaagugccauuucgccuccuugugucacauagggag | |
| | aggggucuccagggccugaaacuaaaaccgggugagaucgggcaacugcccguaga | |
| | ucacucagagaugaaauguuggcacaaccgggauccagaaucuucaucauucccaa | |
| | guuggcuuccaugacugcaacagauguuuucagcuguuggauuucggaccgcauc | |
| | auagggauggaggaugucuguuucaagacaagaucuagcugauagucaaccuuac | |
| | uuacucucugugauaucgccuccaucauagacaucaucgcuugcacaaagucuaca | |
| | gguggcuggacaugauccgcagauacaaggguauugucuuggcucugccuugauc | |
| | ggagagcaugagggguugcaccagcugauaguugugacuccucccauuguccaug | |
| | auaugcuguguucacgucugugcccugguuuccaggggcggccuugacuugguuc | |
| | ugcggucuuuccugacuguuuccgcgacuggguugacuccccugcuguugagucg | |
| | gacguuggugauuccccucuugggggcucgaccaugggcccuuuuuagcauugga | |
| | cgauuuauugcugagcuugucaagcaucaacagcagagaguugcuugcuccgguc | |
| | cugagcugugugucgacggcuucgucuguggccuggguggggggcuggucggcg | |
| | gauguggccggcgggcugucaugcggggucguuugcucggguguggaugguugu | |
| | uugucagaucuguccugucgaucgggguugucuugacuggccgguggcuggaug | |
| | cucccaugcuucucccaugcugcgcucagcaccuuggucuugccuugugggauug | |
| | cacuccuuccaacagucucugcugguuuacccugggcuguaauuauguugucaau | |
| | gacaguuccacuugucucaaauagcucgucgaucucugcaucuguaaagguggcca | |
| | uguuuguccuggucuaucagguagaggagagagcagagacucgggagacuugccc | |
| | ugaucucugaauaucccucuucuacccguacuuuuuucuaauucccucggcucug | |
| | uuuugauuguuagugagccgcauugugccugugguaucuagggcguacguaguu | |
| | guacagcagggggagggaggaaagagggggauguuuguuugaggguguggucau | |
| | auagagccgcaaaucgaggggucgacuacgggugagggcauugggauguuuuugu | |
| | ggaagcaggcuggguuuuguccaucaauacccccagucggugucguuaucuuggg | |
| | auggcccaggaguugggggaggccccgauuggggagugcccugugcagaguuugg | |
| | cgccucccucaugcuauuugcuaccgcucucaucagauccaggaauugggucuccc | |
| | cauccccggcuucugguugcccuugcgaucuauucgauccgccuuguagagcuug | |
| | ggacccccccucgcuaagcccagugaggacuccgacuuguugaguaggcaugucua | |
| | ugcugcuggucuccucggagacccguugggcagcagcugccaggccccuccuugc | |
| | ugcugggguuagcuuuagcucggcagccauauccucguuaaugcuacuucccuga | |
| | gccugagcguacucuacuccaagucuccagaaugaugugcucauaaagucccuggc | |
| | aaauugguauuucccaguaccuuuaucuaggacugaugccauacccauggcaaagg | |
| | aguaaaguugugcauacucggcaggcgcaaagcucaucuggucacuaucaccaagu | |
| | aaugucauguacggcgcauuaucuccuuucauccgauacaaacgcaugagcugcuu | |
| | caucuucuggaugucgccugagaggcuacuaagugcaagggcugaugucuuggug | |
| | uugauuccguacuugagugucaagaagaaugcaguaagcccgguauuccugaugu | |
| | augagucuacguccccuaccagguuauaauaaguagagguaccaccugccguguug | |
| | cggccucucuugagcucgcuaaccaaaaagaugcggacugcaagagacugucugau | |
| | cgugaguuggauugugcuccugcauacgggguagaggauguauuucuuuuggac | |
| | ccugccuugcugcauauacuuauugauucgccuuguuuccgacucaucugcaguc | |
| | ucauacgcagucauggcuuuugcuacugugacccauacuugagccuggauagaga | |
| | ggauccucuccaggguaucggugaugucuucuggugcaucaucuucggccccggc | |
| | ugugacgaacgggguuccguugcugcaugcccgagggagagauccugcuaucauc | |
| | gcaaaucucugugcucucucuucagacacuccacuccuauuguugaacuggggcg | |
| | ugccguuggcaaagccaucaaucucaagcacggccaauguggcuucauucuguuuc | |
| | ccugcaagggcaacaugguuccucauuaccugugagugggagcauaaaagagauau | |
| | gagagcaccuugccugagugguuuguuggcaucuucgcuaacagcaauccggagg | |
| | cagaauaccacaaagcuccaucuaucuucugggucaucacuguuaagagugaauac | |
| | cgggacgucuacuuuuaagguacucccuuuuucucccccuccaugagcuccauug | |
| | gggcgagucugagccgcgaggagcuguucguacucaucaaauacggaagacaugu | |
| | uggcagaaggcuuucucgaguuugugcuucgggcucgcacucgagauucacaccu | |
| | ucuacccgugcgacuucaauugcuccuucgccuuuuaucguaacucacggauucuc | |
| | uguuuggu | |
| Exemplary | accaaacaaagauuuggugaaugacgagacuacacucaagaauaauugugcgcaac | 51 |
| NDV-huIL-12 | cuuuuuuuaagacauuuauuugaguucgaauucgaguccuaaggagucaggguuc | |
| genomic RNA | aacuuuuuucuaacauaauaugauuaaaucaacaagaauacaauuggccaaaaaag | |
| sequence | gagccuauuaauaugugauuuucguuaagagucacaguuacuguaauauccuuug | |
| (Version 1) | acugcauugccuauaguuuucauguagaauuuuuguugagcacgagucaaguaca | |
| | guacagaagugucuguaaacauuucuuugaguuugguaauaccuaggacagccuu | |
| | caaauauuuagggcagguacuaugcuucaaguauguccuuagugggauaaggucc | |
| | uccauggagaucaugccuuuaagcacuaggcugauuauaucgccuauuuuauuga | |
| | gauuuucgacucuaagaccuaguauuguaaccucuaggaucugucucgugcacug | |
| | uuuaagugauguccuuuuuuucccgucaguagagagauuguaagggguaaauaga | |
| | aauacugugucagcgagaucacccucagcuuccauauauaucacagaccggauaac | |
| | ugugucaaugugacuagcgaugaucuggguuaucugaguuauguucgcuagcgu | |
| | gcucaccagacucuccgcacagaauggacggacgggcugucccccggcuucaauca | |
| | gcgcagugucaauauucuuccucaaguacuuuauuaaucuugguaaaggacugga | |
| | uaggaugucugucacacgcugccgcugugaggugaauaaccuggucagugugauc | |
| | ucaucugauuuagacaaaagcguaccgugccgcugcaccagaguuuucgccauccu | |
| | caccaccucauguacaaauguaggcccgcccagguaacccaugacaaauaccaggua | |
| | acacuccauaucuccucgacaugcauaaccauuagagagaauauauccuuuugugg | |
| | aacacggagcaaacaaguucaugaguagaugaaaguaguaucccauugcauacaac | |
| | acuuugaugauuacuaccccgcccucccuuacagaaugcauggcaaucagagauaa | |
| | auugauagcuaguugaucuaguaagcuuugauuggacccuggaggaauuucaaug | |
| | ucacaaugcagcaaugauacagaucuguagggcacugcagauguaauauaccccac | |
| | uacuuuaucugaggucaggucgcuuuccucuguauuuucucuccauaauggacgg | |
| | aacucuuggacaaauccaucuuugcauguuaccuccgccuguagauuccuauaaac | |
| | aaccgaauucaaaaacugaguuggggucggcccgaaaugucguugcgggggguuc | |
| | aucucauuugaaaagagcguauuguaauagauaguuucaugugguacaugcaguu | |
| | cgagaagacucaugauggcuccgcucccuucagcuaaguauaaggaguucccgugu | |
| | cuugcacaucuuaccucggguacagaaaggagaugagaugccuuauaccaagagga | |
| | agaugcagucccuaucccucugaacaaguaucguacuaaguagucuuccuccggau | |
| | uuggagaugugaguucaggaugaaucugacuaagugugaaugcgucauaccuugc | |
| | uggagcacucaaaucuaacucuugcaaaaaugccgcagguugucgggugaaugga | |
| | ucuuucacucgagcaccaauaucuugcacagaagggaacucuaauaauggcucuug | |
| | ggggaacaacaacgccaggauaguaucccuguccucccuuucccugaucaaauuga | |
| | ggcucuuccgagacauguaguacagauuagcugggaauguaauuauguuaggaga | |
| | caugauagagcucacuugaucggggcuaaguaaugguuucacagcauccgacagua | |
| | aauacucagugaguauugaacauuucucuucugcaguuaagccucuuauuuucgg | |
| | gauuucucuuguuguagcaaagaguaccguguacagacagcauaaccgggauauca | |
| | gcugaagcaucuucucauucagguuaucaucuaagacagaugggaacagcagauca | |
| | uacuuauuuccugaauauaagccggagaucacacgucgggugcaagauacuaauag | |
| | uuuugcagacauuucgauaaaauccguaucugcaaguuggugugauccgucaugg | |
| | uugaccaggcccacugcauguaaccuugaaugaaugacgggaugagauauuguag | |
| | cugcaauguuggaaaguagaauuccuggcauauucuuguauaaaucacccauauau | |
| | aagacaauauuguccaggccucuuacucucagguaauagaguugguaagaacaguc | |
| | gaggagcacuucaagugcugcauauucaaauaggcggaccacaucugaauucugag | |
| | cuucacugauccaauuucggguauugucauacacuauuauggcgucauucuuuau | |
| | ggagguaucuucaucauaagaaaccacagacuggccaaucaacuucccgcuggaua | |
| | uugaaagaauguucauuagcucuaucgugggauaugacuccagauuaagcucaua | |
| | acucuugaagauagcuaagucaagucucgcaaagucucccuccgauacagggcuag | |
| | gaucauacauaaacuuauuugaggucacuguccucaguuccgguaccaccccaagu | |
| | agcucgaaaggaaccgcaacaggugcuucucugauacagcaacuaaauuuacuaug | |
| | uaggugcagugugaucucaucauauguccugguuguugucauuggaaagaucgau | |
| | ucgauuagagauaaacccaagagcaugaucuguugguaaaccacauuccccucuuu | |
| | gacuccuucuucagugaacagccuuugagaaucauuggauaugugaauguaaggu | |
| | gacacccuguagagagaugcaggggugaaugucaucugaguuauaccaucaucua | |
| | gucuauguugaagauucccagccguggguaaaggggacaguaaccgaagauacuc | |
| | uaaguuuacauuacaccgagauuuugcaaucguaagagcagcaguccaauuuacuu | |
| | cauuauccccauaagcccagaucaacacggaugaugcccuuagggcagccuuuaca | |
| | uguggcgacauaugagcuauuuuugcaagugaggcagcucuccucuccugugucu | |
| | uugacccgagauaugguacccucaucggaggauucuugcuggugucaucggucaa | |
| | uucuauauugcuuggaagauggaaccaaguaaauuguucaucuccgcugucacau | |
| | cuuguacacccuccgcuuacacuaagaaucucacccucuacgaguucuaucguauc | |
| | aggauuagauacacccaguauuuuccugccucccgucaaaggugaccagcuucuau | |
| | uccgugcauagucugccagugucagagaacacauauuagaagagacuaaggggug | |
| | guuggaucuacuggaggaaaaaacaucgucucuaaacagcauugcaugcaugcuag | |
| | aauaauugacuauccgcaucagccucuugaugccuaauggccuccuaguaagcgca | |
| | aucuuaauuacgguguuuguugugucaacaagcccuugaauuugcuuucuccuac | |
| | cuacagagcuugccuccaugauggcaugcgcaacgcggggaugaaucaccucuuga | |
| | uuaagcaagaauucagccaaugccuucucuucugccucauuauccucugugugcac | |
| | uccagacaauaagggauuugaacaaguuucaaauaggacucuuugcguauguuuc | |
| | uuaagaacaauauuugggcuugcaacagucucaaaauugaaagaguaugggucgu | |
| | ugcacagacuggcccaaucuccauucccaggcggccuaguuaagauauuagucaua | |
| | auguuaggacucaguaaucccacugcuucuagucgcuugaucucugcaaaagcagu | |
| | aguccccgggucaccgauauuucuaguguagagccuugaguauugaagguuacuc | |
| | agucccccuaauugggcaggagucagaacauaugagugcacaaaagagauguccuc | |
| | aauccacgacugauuaagaucggggugcgaauuguuggugauggagaacucagag | |
| | ucaaaguaugucugcacacaacucauuauauaguuuaaauaguaacagaagucuuu | |
| | gggaagcccguucucgcauagccgugcuacaguagaggcaauguuggcacaggaca | |
| | uuacgguguuuucacugagaucaccugacacuagcacuaauuuagaugaauuuuu | |
| | gaggacuugacugaggauugcuccaucuuugaagauucguuugcuguauaugaag | |
| | aaugugucugaccugaugguuucacgauccuucaaauuauggccaaucaaaugau | |
| | ugacaugaauuaauuccuugaagaaauuaucacuggcuugaugcaacugugucaac | |
| | accaucuccggagagucgucugaucuuaccucucucguuacugcuauuacuugau | |
| | uaucacccuguaccauacaggcaacacgacaaugcgaucuagcugcagcaaguugg | |
| | auugcagcaauugagaucauuguccauagcuucuggcauaauccuucgauaccccc | |
| | ucuggcacugacaauauauaugucaucauuagggacucuugagaggucacaguca | |
| | guagggucacuuggaggauugaaagggucuccuacgaacaucguaguguccauca | |
| | gucuuaggugaauccauucgaagaagugagguaggcccaucaacugauugauggc | |
| | augagcgaacaauuugauugucugauaucuccaauuaagacaguacuuuugcagg | |
| | ucaguuguuaugaagguugcaacucuccgacgguucuugcuuuucggaucaugau | |
| | ugcgguuugaagauacucuuucuuuacagucagugauacguuucuuauugcuguu | |
| | aaaagacaguugacucaucgcuagcauacucuuggucaaggauaugcuauccugaa | |
| | ugacuccauuucccugaaagaaaggugcaaucugaucggcuaggaucccuuccgcc | |
| | aucaccugacaguuccuuaacuucuuugucagcuuagcgaagauccguccauuaac | |
| | uuucacuuccuucuccuugagcgaguaugauacugccacauugucaucucuaagg | |
| | uacucaagggucgucagauauuccaucucuuuauauggaucaaaaucauuugacuc | |
| | uaaaaacucuaucaagaggcgauuagucgaaguugcuucuuuuacauguuucuuc | |
| | uggucuucggagagaagguuccgccuaaacgaggcaagccaauuaucguuggggu | |
| | gugcgauugccuugucuuuuaggaacaugcucagguuggugacagggucauauuc | |
| | uauacauggcucaaauucaagugcagauaaacucuuauacucucucaacaugauau | |
| | cgugugaaaucucugcugaaucugcauguaguugcccaaugaccuucccauauau | |
| | uguauccacuuugacucgcggccacacaccugcauucuucuuucuguacccguuga | |
| | ugauuguucccuugaagaaagacaguaccugaaggaucauaucaaagucuaccauu | |
| | uucggugcgcacauuuggcuccugacugccuuugcugcaauacgggacucaagca | |
| | gugggugaccccacagacgcaacagacacaacaucucagcugcuugauucuguucu | |
| | aaaccagagaauacaguagcgauugcaugagucacggauucugcuauaucauuggg | |
| | gaggaggccaauuagaaugucuuuaagcuccugcagguugaaugcgaagaaaucu | |
| | ccugcaaauguaccugacggcucgaguagcuggacagcuccguaugcaaaucccuc | |
| | cauuagugauacaacaucguagacuugauuacccaagucuuuugccagagcgucua | |
| | uuaaccgcaaaaugucaucaauuuucucugauaagcuucugagaugcaccgccgug | |
| | guugauauuauguugaccauaucucugcccuccaucauaucugcauacaucaauac | |
| | aaguuccuggguaagacaugugaacuuguucucauucguaugcgucacaacgacaa | |
| | guucaggagugacaaagacuuggccuaccuuauggguuagcaucaccaauuuguu | |
| | ggccgcagaccuuguccuagcugccaccaucagaugccucuggaucuguuuuaua | |
| | uggagccaugcaaacuuggcuguggaccauuuugagugaaaccagaaugccggau | |
| | ccguacgaaugcugcugaacuccucugaccgggggacauuguuagaccaagaugac | |
| | cccagcaguuucuucucuauaugcguacacagccuugugaacaguucuccauaucu | |
| | cguguugugaauuuggaucuucuucucaaucuuccgaaauuuguugguugaaucu | |
| | gggaccucaauauuagccaguucuucuaaacaccugggguggagcacuccgguua | |
| | uucuggaauugugguuaagaguuugguguacugcccuuccgaguuuuaucauuc | |
| | ucucaguaucaggagaggccgauucaaguauuuuuuuccauugucggcugagaau | |
| | gagguggucgaagucacauucaucaggaagcgguagcccaguuaauuuccaguaa | |
| | uagaguaguuugugcuugaccaauggugaagacaggugugacucugguaggauaa | |
| | ucugaugcucugcccuuucaggaccggagcucgccauguccuacccguauuguua | |
| | accaugagcuguuuugccuuguaucucauugccacuuacauuuuuucuuaaucaa | |
| | gagacuauugacaaggcuugaucugaucgcaugagcacuggcugauuguggucaa | |
| | cuggcaacauggaauucgagcacgcgccggcauucgguuugauucuugaugucgc | |
| | agaagauaggugauacaaugccaucuuuccaacuccuuuauaauugacucaacuag | |
| | ccagaccuggcuucucuaaccccgucaucuuugaggaucucaacuaguaacgggac | |
| | gauucugaauucuccgaagagaguauuagauauuucagcaaugcugagacaauag | |
| | gucuuauuagucuugaccacuuuaaaacaaguugauguuguguaugcugcuuugg | |
| | uacugcuugaacucacucgaguaaugcgacugcgggaugugcuaucgaauacugc | |
| | agacgcaggguuaagucuugcuuguacaccaucaagcauugucccgaauaccccuc | |
| | gcaaggugugguuucuauagaagauuaggggauauggaucuguauagacuccagu | |
| | aacacacgaguuggggcaucuugcugaagccuggcaagggauacuaccuggccgag | |
| | ugaaggcauugaauguauaaggacuaugaagaguggcuguuuuguugcugacugu | |
| | cauaggauauaauaacgcgggagagaaguaugaugacccucguugauacaagaaau | |
| | gagaugucccuacugugagaauucugccuucggcccccaugagugugacuguguu | |
| | gggcgguacagucaguaccgggucuucgccuaaggauguugacaccuugauagau | |
| | aagauagccugcuguaugcguuucccaccaaaccguccaggcuuauacgaagacuu | |
| | ggccauucgaaucugguagucuugcucaucugggcaugugucauuguaucgcuug | |
| | uauaucacauauuucccuuccuguacagugucacugggugaauuggguuuuaacc | |
| | cuccguagacugagaaccauacgcggcugucaauaaaagauccacccccuacuccu | |
| | ggguaguuggccacccaguccccgaauaauguugugacaucuagguccuuuucgu | |
| | gguacuggccgucgaacccuaaccucccauguaccauccgcguagggacagcugag | |
| | uuauaaucuucuuccucugucuccgugacuuucgagcacagcauaucacaacccag | |
| | gggaguugcacucacacugcaagacuuccgauuuugggugucguccagguugaug | |
| | gaacgcagaguagaaaagaauacccucccuguugcagauguccggagcacaccaag | |
| | ugcuaaauacugauaugaaugugagugaucucugcauccagacaauauuacauuau | |
| | ggguguagcaguaauggguagcacucaugucaaaugaggguauucgagugcaacc | |
| | ugauccuguaguaggcgccgggauaaaauucagauguucuugaaaugcagaggga | |
| | uagaaugaugugacaucacuagcaucaucuacaaugaguucuuugccuaucccccc | |
| | uauauaaucugggucauggauaggugccccccacccacuguuguuugcagcucca | |
| | uuaaucugauaagagagagauguuauugcguucauaauuguggucucaguauuua | |
| | acaaugccaacggagacucaagggccacuugcuuauauauccuaucuacuacaucu | |
| | ugauuggaaccaaguguagauguaaucuuuucuucugcccuggaaauccuagucg | |
| | guaugccuacaagaucgcuaggugugcuagcccccaugcuauauaaaagggaggcu | |
| | acagauauagccaaggucacuacuguuaagaauaagauugcaauccggaauaucaa | |
| | gcgccauguauuuuuugccucucuuucaucauucucuaacgcaacuuggcuaacg | |
| | gcgcgguccaugauugaggacuguugucggugaagcgguagaacggagguuguga | |
| | agccuggcucgcaauugaggggcggccucucuuaccguucuacccguauaucguc | |
| | cuuuggucaucuacaaccgguaguuuuuucuuaacucucugaacugacagacuacc | |
| | agaacuuucacacaaauuacuauuagggaaaccuucguuccucaucuguguucaca | |
| | uuuuuguaguggcucucaucugaucuagaguauuauucccaagccauaauaaggu | |
| | cuuuuguugcgccuuuugcuuguacauuagguagcaugcuagaaucaggcuaagu | |
| | auaccaaaaacaagagauaugauagucaaaacgauauagguaaugagagcagaugu | |
| | gcuagucaguuugacauugacuuugucuaguuuucuguugcuuuccucuaacuua | |
| | uucaaagcauuacugaucgaguuguugacauucccaagcucaguugagauaucaag | |
| | auugccuguuauuauuacuugagaaucuuguauugagauauucuucugauaaguu | |
| | acaucgaauuccccacugagccuuaaaguuaucccgccuaaggauaaaacauugca | |
| | ugauuguuuaucuauuagagacacggcuucuccauaguuuugcgauaugauaccc | |
| | gggggguuuacacaucuacauguugucaucuugcaguuggcgaugacugaaccuu | |
| | ugauagucauguaugguguaguaagugcgccuucggucuuugaguacauacaggc | |
| | cgacguauugccgcucaagcaggaauaaauaccaggggacauagggaacguuacua | |
| | uucuuguacaauauaaaucuaagucaguuucuauacaguaugaggugucaaguuc | |
| | uucuaucacagaaccgaccugugucaccacuuuugggacaagugccgaggcaaauc | |
| | cccugguugugcuuacggauaagguuuccaaguagguggcacgcauauuauuuag | |
| | guucccgacugaaggggcaguuaccuguauacccaagaguugagucugugagucg | |
| | uauagaauaggguuaccggugauuaagccgcuaccgauuaaugagcugaguugau | |
| | uguucccuacaccuaacuuagucaauaaguaauccauauuuccaccagcuagauug | |
| | uaaagugccugaauagucagcuuguuuaaagcaggugaagugauuugugguccga | |
| | auacuguagucaauucgguuagguacagguugagcucuacaccaacuugcugugc | |
| | aauuuugaugcagucuaauuccugagcuguuuuauuaaauuggucauuaacaaac | |
| | ugcugcaucuucccaacugccacugcuaguugcgauaauccgucagugaccucaug | |
| | cacagccucauugguugcggcaaugcucucuuuaagucggaggauguuggcagca | |
| | uuuuguuuggcuuguaucagagcugcggccgcuguuauuugugcggcaguugca | |
| | accccaagagccacaccgccaauaauggcgccuauaaggcgccccugucuccccccu | |
| | ccagauguagucacagacucuuguauccuacggauagagucaccaagggggguga | |
| | gcaaaguggucaauguccuguuguaugcauccaagggggcuuucgcacaugccuc | |
| | cuuauccuugggcagauucgggaggagcuuaacuaugauugauccugucugggau | |
| | gagguguauauguugacggcuuugucuccuguaaccacaauuccugcagcugcaa | |
| | gaggccugccaucaauggaguuugccggacagaugcaacucaguaccagcgcaacc | |
| | cggauagucagcaucauaggugcuggguucuugguagaaggucuggagcccaucu | |
| | ugcaccuggagggcgccaaccgggauccagaaucuucuacccguguuuuuucuaac | |
| | uugauagacagguaaacuaacuguaaauaaucaagacagauuaguuuuuuguguc | |
| | augaugaucugggugagugggcggagcgcaaucucagagacgcagcuuauuucuu | |
| | aaaaggauuguauuuggcaagggugugccccuucuccagcuuaguagagguaacc | |
| | ucguggucggcggucacugcgacagcgcguuggguaccugcuuggauaaugauuu | |
| | uaacgcuccgcaggcacgcgguuugacuccagaguaucuuggccaccugaggagaa | |
| | gcauuugcuaugggauagcaggcugucccacugcuagagaagaaaggugccaaaag | |
| | cuuaguccgugcaccucuugcuuuuaccaacacggaaggcccgagcacaucacuga | |
| | gcccgacagauagaucaaggcuccuuauuuucuuuuccagcuugucaaaugucacu | |
| | uucuuccccuuccuaucuacaguggucauaaguccaauaugcaagaagagguuagc | |
| | auaguauccgcugucagacuuagacagagauuuaaccaaaggacuccucgggucua | |
| | ccuccacauuaauagugacauugagcgcaagauuguacagacucgagccagaaacc | |
| | uucaauacugcagcugggaucuuguagacaucccucuucgguaccacagucaagga | |
| | gacaaaguucaccuuguauucuaggguuccacucccgggaaucuucucuggcgcu | |
| | uucacgugcuugacugcauucacugaugaguauuuguuugccacaacccuacagc | |
| | uuugcagcacuuggggugccugcacuacugagaaaaccauucucucaguauuagu | |
| | ugcacucuucuugcaugugacuaucauagugagacaggcccuugccagcucaauaa | |
| | ggucuccgguauuugggacgcuuccuaggcagagcaucgcagcggaaaguaacuc | |
| | gcgcuuggguuuaucaucgaucaugccgacgguggcuucuucauucccaacuuga | |
| | aagaugaauccauagguggugaugaauacugaguccuccuuacuaucaguccacaa | |
| | gucaaggcgcuggauccuauauugcggggcgauuugcuucuucccaucuccugug | |
| | ucuuguaggacgaucggaaaugcuaacagguugcuagaagaaugggcagaaucaaa | |
| | guacagcccaauuguccuagaugaguccaucuuggcacaauuggggcacuccaauu | |
| | cuacccguauuuuuucuuaaucuuaaauguagcuagauuaauuacgguuacgcga | |
| | ucauucaguggggcugaggaagcgagagaggauugccgcuuggagaguuggaccu | |
| | uggguccgcggaucauuaggaagcauucagauagcucaucacucuaucaauaguca | |
| | cugcccgaauucugaaagcaugaagaaguaugcagagcuugauuuuaguuuuaua | |
| | aaaauccgguucuucaagggaggauuuuuguggcacagucucacuguugaaauuc | |
| | agggccugcaucagcucaucaauaacugccagcauguuuugaucuagaaagaucug | |
| | ccucuuaggauccaucagaagcuuugcauucauggucuugaacuccaccugguaca | |
| | ucuucgagucuucauaaauacuacuaaggcacagggccaucauaaaagaggucuuu | |
| | cuggaggccaggcaacucccauuaguuaugaaagaggucucucuggaauuuaggc | |
| | aacucucauucuugguuaauuccaaugguaaacaggccuccacugugcugguuuu | |
| | aucuuuugugauaucuucaugaucaaucucuucagaagugcaaggguaaaauucu | |
| | agaguuugucuggccuucuggagcauguugcugacggcccucagcagguuuuggg | |
| | aguggugaaggcaugggaacauuccugggucuggaguggccacggggagguuucu | |
| | agauccgccgccaccgccaccacugcagggcacagaugcccauucgcuccaagauga | |
| | gcuauaguagcgguccugggcccgcacgcuaaugcuggcauuuuugcggcagaug | |
| | accguggcugaggucuuguccgugaagacucuaucuuucuuuucucucuugcucu | |
| | ugcccuggaccugaacgcagaaugucagggagaaguaggaauguggaguacucca | |
| | ggugucaggguacucccagcugaccuccaccugccgagaauucuuuaauggcuuca | |
| | gcugcaaguucuugggugggucagguuugaugaugucccugaugaagaagcugcu | |
| | gguguaguuuucauacuugagcuugugaacggcauccaccaugaccucaaugggc | |
| | agacucuccucagcagcugggcaggcacuguccuccuggcacuccacugaguacuc | |
| | auacuccuuguuguccccucugacucucucugcagagaguguagcagcuccgcacg | |
| | ucaccccuugggggucagaagagccucugcugcuuuugacacugaaugucaaauca | |
| | guacugauugucgucagccaccagcaggugaaacguccagaauaauucuuggccuc | |
| | gcaucuuagaaaggucuuauuuuuggguucuuucugguccuuuaaaauaucagug | |
| | gaccaaauuccaucuuccuuuuugugaagcagcaggagcgaauggcuuagaaccuc | |
| | gccuccuuugugacagguguacuggccagcaucuccaaacucuuugacuuggaug | |
| | gucaggguuuugccagagccuaagaccucacugcucugguccaagguccagguga | |
| | uaccaucuucuucaggggugucacaggugaggaccaccauuucuccaggggcaucc | |
| | ggauaccaauccaauucuacgacauaaacaucuuucuucaguucccauauggccac | |
| | gaggggagaugccagaaaaaccagggaaaaccaagagaugaccaacugcuggugac | |
| | ccaugguggcgguucuacccguauuuuuucuaaccgcgggggggaacucggugca | |
| | gauuccgugugaugccgaguggacagggacccgcuacguguggcaguaguaauua | |
| | gccauuuagagcaaggcgcuugauuuuccugauuuccucgaucgacccggcugca | |
| | ucuaacuugcuuaggagcuuggcugaagaacucgggugcauugggcgugacauga | |
| | ucaaugcacggacaguguccuuuuccacuccuauaucaggcccgcaugcaguggcg | |
| | gguuuaaucaauucagauggauguggcacugguugcgaaaguuuauuaagugcca | |
| | uuucgccuccuugugucacauagggagaggggucuccagggccugaaacuaaaacc | |
| | gggugagaucgggcaacugcccguagaucacucagagaugaaauguuggcacaacc | |
| | gggauccagaaucuucaucauucccaaguuggcuuccaugacugcaacagauguuu | |
| | ucagcuguuggauuucggaccgcaucauagggauggaggaugucuguuucaagac | |
| | aagaucuagcugauagucaaccuuacuuacucucugugauaucgccuccaucauag | |
| | acaucaucgcuugcacaaagucuacagguggcuggacaugauccgcagauacaagg | |
| | guauugucuuggcucugccuugaucggagagcaugagggguugcaccagcugaua | |
| | guugugacuccucccauuguccaugauaugcuguguucacgucugugcccugguu | |
| | uccaggggcggccuugacuugguucugcggucuuuccugacuguuuccgcgacug | |
| | gguugacuccccugcuguugagucggacguuggugauuccccucuugggggcucg | |
| | accaugggcccuuuuuagcauuggacgauuuauugcugagcuugucaagcaucaa | |
| | cagcagagaguugcuugcuccgguccugagcugugugucgacggcuucgucugug | |
| | gccuggguggggggcuggucggcggauguggccggcgggcugucaugcgggguc | |
| | guuugcucggguguggaugguuguuugucagaucuguccugucgaucgggguug | |
| | ucuugacuggccgguggcuggaugcucccaugcuucucccaugcugcgcucagca | |
| | ccuuggucuugccuugugggauugcacuccuuccaacagucucugcugguuuacc | |
| | cugggcuguaauuauguugucaaugacaguuccacuugucucaaauagcucgucg | |
| | aucucugcaucuguaaagguggccauguuuguccuggucuaucagguagaggaga | |
| | gagcagagacucgggagacuugcccugaucucugaauaucccucuucuacccguac | |
| | uuuuuucuaauucccucggcucuguuuugauuguuagugagccgcauugugccu | |
| | gugguaucuagggcguacguaguuguacagcagggggagggaggaaagaggggga | |
| | uguuuguuugaggguguggucauauagagccgcaaaucgaggggucgacuacggg | |
| | ugagggcauugggauguuuuuguggaagcaggcuggguuuuguccaucaauaccc | |
| | ccagucggugucguuaucuugggauggcccaggaguugggggaggccccgauugg | |
| | ggagugcccugugcagaguuuggcgccucccucaugcuauuugcuaccgcucuca | |
| | ucagauccaggaauugggucuccccauccccggcuucugguugcccuugcgaucu | |
| | auucgauccgccuuguagagcuugggacccccccucgcuaagcccagugaggacuc | |
| | cgacuuguugaguaggcaugucuaugcugcuggucuccucggagacccguugggc | |
| | agcagcugccaggccccuccuugcugcugggguuagcuuuagcucggcagccaua | |
| | uccucguuaaugcuacuucccugagccugagcguacucuacuccaagucuccagaa | |
| | ugaugugcucauaaagucccuggcaaauugguauuucccaguaccuuuaucuagg | |
| | acugaugccauacccauggcaaaggaguaaaguugugcauacucggcaggcgcaaa | |
| | gcucaucuggucacuaucaccaaguaaugucauguacggcgcauuaucuccuuuca | |
| | uccgauacaaacgcaugagcugcuucaucuucuggaugucgccugagaggcuacua | |
| | agugcaagggcugaugucuugguguugauuccguacuugagugucaagaagaaug | |
| | caguaagcccgguauuccugauguaugagucuacguccccuaccagguuauaauaa | |
| | guagagguaccaccugccguguugcggccucucuugagcucgcuaaccaaaaagau | |
| | gcggacugcaagagacugucugaucgugaguuggauugugcuccugcauacgggg | |
| | uagaggauguauuucuuuuggacccugccuugcugcauauacuuauugauucgcc | |
| | uuguuuccgacucaucugcagucucauacgcagucauggcuuuugcuacugugac | |
| | ccauacuugagccuggauagagaggauccucuccaggguaucggugaugucuucu | |
| | ggugcaucaucuucggccccggcugugacgaacgggguuccguugcugcaugccc | |
| | gagggagagauccugcuaucaucgcaaaucucugugcucucucuucagacacucca | |
| | cuccuauuguugaacuggggcgugccguuggcaaagccaucaaucucaagcacggc | |
| | caauguggcuucauucuguuucccugcaagggcaacaugguuccucauuaccugu | |
| | gagugggagcauaaaagagauaugagagcaccuugccugagugguuuguuggcau | |
| | cuucgcuaacagcaauccggaggcagaauaccacaaagcuccaucuaucuucuggg | |
| | ucaucacuguuaagagugaauaccgggacgucuacuuuuaagguacucccuuuuu | |
| | cucccccuccaugagcuccauuggggcgagucugagccgcgaggagcuguucgua | |
| | cucaucaaauacggaagacauguuggcagaaggcuuucucgaguuugugcuucgg | |
| | gcucgcacucgagauucacaccuucuacccgugcgacuucaauugcuccuucgccu | |
| | uuuaucguaacucacggauucucuguuuggu | |
| Exemplary | accaaacaaagauuuggugaaugacgagacuacacucaagaauaauugugcgcaac | 52 |
| NDV-huIL-12 | cuuuuuuuaagacauuuauuugaguucgaauucgaguccuaaggagucaggguuc | |
| genomic RNA | aacuuuuuucuaacauaauaugauuaaaucaacaagaauacaauuggccaaaaaag | |
| sequence | gagccuauuaauaugugauuuucguuaagagucacaguuacuguaauauccuuug | |
| (codon- | acugcauugccuauaguuuucauguagaauuuuuguugagcacgagucaaguaca | |
| optimized) | guacagaagugucuguaaacauuucuuugaguuugguaauaccuaggacagccuu | |
| (Version 2.1) | caaauauuuagggcagguacuaugcuucaaguauguccuuagugggauaaggucc | |
| | uccauggagaucaugccuuuaagcacuaggcugauuauaucgccuauuuuauuga | |
| | gauuuucgacucuaagaccuaguauuguaaccucuaggaucugucucgugcacug | |
| | uuuaagugauguccucuuuuucccgucaguagagagauuguaagggguaaauaga | |
| | aauacugugucagcgagaucacccucagcuuccauauauaucacagaccggauaac | |
| | ugugucaaugugacuagcgaugaucuggguuaucugaguuauguucgcuagcgu | |
| | gcucaccagacucuccgcacagaauggacggacgggcugucccccggcuucaauca | |
| | gcgcagugucaauauucuuccucaaguacuuuauuaaucuugguaaaggacugga | |
| | uaggaugucugucacacgcugccgcugugaggugaauaaccuggucagugugauc | |
| | ucaucugauuuagacaaaagcguaccgugccgcugcaccagaguuuucgccauccu | |
| | caccaccucauguacaaauguaggcccgcccagguaacccaugacaaauaccaggua | |
| | acacuccauaucuccucgacaugcauaaccauuagagagaauauauccuuuugugg | |
| | aacacggagcaaacaaguucaugaguagaugaaaguaguaucccauugcauacaac | |
| | acuuugaugauuacuaccccgcccucccuuacagaaugcauggcaaucagagauaa | |
| | auugauagcuaguugaucuaguaagcuuugauuggacccuggaggaauuucaaug | |
| | ucacaaugcagcaaugauacagaucuguagggcacugcagauguaauauaccccac | |
| | uacuuuaucugaggucaggucgcuuuccucuguauuuucucuccauaauggacgg | |
| | aacucuuggacaaauccaucuuugcauguuaccuccgccuguagauuccuauaaac | |
| | aaccgaauucaaaaacugaguuggggucggcccgaaaugucguugcgggggguuc | |
| | aucucauuugaaaagagcguauuguaauagauaguuucaugugguacaugcaguu | |
| | cgagaagacucaugauggcuccgcucccuucagcuaaguauaaggaguucccgugu | |
| | cuugcacaucuuaccucggguacagaaaggagaugagaugccuuauaccaagagga | |
| | agaugcagucccuaucccucugaacaaguaucguacuaaguagucuuccuccggau | |
| | uuggagaugugaguucaggaugaaucugacuaagugugaaugcgucauaccuugc | |
| | uggagcacucaaaucuaacucuugcaaaaaugccgcagguugucgggugaaugga | |
| | ucuuucacucgagcaccaauaucuugcacagaagggaacucuaauaauggcucuug | |
| | ggggaacaacaacgccaggauaguaucccuguccucccuuucccugaucaaauuga | |
| | ggcucuuccgagacauguaguacagauuagcugggaauguaauuauguuaggaga | |
| | caugauagagcucacuugaucggggcuaaguaaugguuucacagcauccgacagua | |
| | aauacucagugaguauugaacauuucucuucugcaguuaagccucuuauuuucgg | |
| | gauuucucuuguuguagcaaagaguaccguguacagacagcauaaccgggauauca | |
| | gcugaagcaucuucucauucagguuaucaucuaagacagaugggaacagcagauca | |
| | uacuuauuuccugaauauaagccggagaucacacgucgggugcaagauacuaauag | |
| | uuuugcagacauuucgauaaaauccguaucugcaaguuggugugauccgucaugg | |
| | uugaccaggcccacugcauguaaccuugaaugaaugacgggaugagauauuguag | |
| | cugcaauguuggaaaguagaauuccuggcauauucuuguauaaaucacccauauau | |
| | aagacaauauuguccaggccucuuacucucagguaauagaguugguaagaacaguc | |
| | gaggagcacuucaagugcugcauauucaaauaggcggaccacaucugaauucugag | |
| | cuucacugauccaauuucggguauugucauacacuauuauggcgucauucuuuau | |
| | ggagguaucuucaucauaagaaaccacagacuggccaaucaacuucccgcuggaua | |
| | uugaaagaauguucauuagcucuaucgugggauaugacuccagauuaagcucaua | |
| | acucuugaagauagcuaagucaagucucgcaaagucucccuccgauacagggcuag | |
| | gaucauacauaaacuuauuugaggucacuguccucaguuccgguaccaccccaagu | |
| | agcucgaaaggaaccgcaacaggugcuucucugauacagcaacuaaauuuacuaug | |
| | uaggugcagugugaucucaucauauguccugguuguugucauuggaaagaucgau | |
| | ucgauuagagauaaacccaagagcaugaucuguugguaaaccacauuccccucuuu | |
| | gacuccuucuucagugaacagccuuugagaaucauuggauaugugaauguaaggu | |
| | gacacccuguagagagaugcaggggugaaugucaucugaguuauaccaucaucua | |
| | gucuauguugaagauucccagccguggguaaaggggacaguaaccgaagauacuc | |
| | uaaguuuacauuacaccgagauuuugcaaucguaagagcagcaguccaauuuacuu | |
| | cauuauccccauaagcccagaucaacacggaugaugcccuuagggcagccuuuaca | |
| | uguggcgacauaugagcuauuuuugcaagugaggcagcucuccucuccugugucu | |
| | uugacccgagauaugguacccucaucggaggauucuugcuggugucaucggucaa | |
| | uucuauauugcuuggaagauggaaccaaguaaauuguucaucuccgcugucacau | |
| | cuuguacacccuccgcuuacacuaagaaucucacccucuacgaguucuaucguauc | |
| | aggauuagauacacccaguauuuuccugccucccgucaaaggugaccagcuucuau | |
| | uccgugcauagucugccagugucagagaacacauauuagaagagacuaaggggug | |
| | guuggaucuacuggaggaaaaaacaucgucucuaaacagcauugcaugcaugcuag | |
| | aauaauugacuauccgcaucagccucuugaugccuaauggccuccuaguaagcgca | |
| | aucuuaauuacgguguuuguugugucaacaagcccuugaauuugcuuucuccuac | |
| | cuacagagcuugccuccaugauggcaugcgcaacgcggggaugaaucaccucuuga | |
| | uuaagcaagaauucagccaaugccuucucuucugccucauuauccucugugugcac | |
| | uccagacaauaagggauuugaacaaguuucaaauaggacucuuugcguauguuuc | |
| | uuaagaacaauauuugggcuugcaacagucucaaaauugaaagaguaugggucgu | |
| | ugcacagacuggcccaaucuccauucccaggcggccuaguuaagauauuagucaua | |
| | auguuaggacucaguaaucccacugcuucuagucgcuugaucucugcaaaagcagu | |
| | aguccccgggucaccgauauuucuaguguagagccuugaguauugaagguuacuc | |
| | agucccccuaauugggcaggagucagaacauaugagugcacaaaagagauguccuc | |
| | aauccacgacugauuaagaucggggugcgaauuguuggugauggagaacucagag | |
| | ucaaaguaugucugcacacaacucauuauauaguuuaaauaguaacagaagucuuu | |
| | gggaagcccguucucgcauagccgugcuacaguagaggcaauguuggcacaggaca | |
| | uuacgguguuuucacugagaucaccugacacuagcacuaauuuagaugaauuuuu | |
| | gaggacuugacugaggauugcuccaucuuugaagauucguuugcuguauaugaag | |
| | aaugugucugaccugaugguuucacgauccuucaaauuauggccaaucaaaugau | |
| | ugacaugaauuaauuccuugaagaaauuaucacuggcuugaugcaacugugucaac | |
| | accaucuccggagagucgucugaucuuaccucucucguuacugcuauuacuugau | |
| | uaucacccuguaccauacaggcaacacgacaaugcgaucuagcugcagcaaguugg | |
| | auugcagcaauugagaucauuguccauagcuucuggcauaauccuucgauaccccc | |
| | ucuggcacugacaauauauaugucaucauuagggacucuugagaggucacaguca | |
| | guagggucacuuggaggauugaaagggucuccuacgaacaucguaguguccauca | |
| | gucuuaggugaauccauucgaagaagugagguaggcccaucaacugauugauggc | |
| | augagcgaacaauuugauugucugauaucuccaauuaagacaguacuuuugcagg | |
| | ucaguuguuaugaagguugcaacucuccgacgguucuugcuuuucggaucaugau | |
| | ugcgguuugaagauacucuuucuuuacagucagugauacguuucuuauugcuguu | |
| | aaaagacaguugacucaucgcuagcauacucuuggucaaggauaugcuauccugaa | |
| | ugacuccauuucccugaaagaaaggugcaaucugaucggcuaggaucccuuccgcc | |
| | aucaccugacaguuccuuaacuucuuugucagcuuagcgaagauccguccauuaac | |
| | uuucacuuccuucuccuugagcgaguaugauacugccacauugucaucucuaagg | |
| | uacucaagggucgucagauauuccaucucuuuauauggaucaaaaucauuugacuc | |
| | uaaaaacucuaucaagaggcgauuagucgaaguugcuucuuuuacauguuucuuc | |
| | uggucuucggagagaagguuccgccuaaacgaggcaagccaauuaucguuggggu | |
| | gugcgauugccuugucuuuuaggaacaugcucagguuggugacagggucauauuc | |
| | uauacauggcucaaauucaagugcagauaaacucuuauacucucucaacaugauau | |
| | cgugugaaaucucugcugaaucugcauguaguugcccaaugaccuucccauauau | |
| | uguauccacuuugacucgcggccacacaccugcauucuucuuucuguacccguuga | |
| | ugauuguucccuugaagaaagacaguaccugaaggaucauaucaaagucuaccauu | |
| | uucggugcgcacauuuggcuccugacugccuuugcugcaauacgggacucaagca | |
| | gugggugaccccacagacgcaacagacacaacaucucagcugcuugauucuguucu | |
| | aaaccagagaauacaguagcgauugcaugagucacggauucugcuauaucauuggg | |
| | gaggaggccaauuagaaugucuuuaagcuccugcagguugaaugcgaagaaaucu | |
| | ccugcaaauguaccugacggcucgaguagcuggacagcuccguaugcaaaucccuc | |
| | cauuagugauacaacaucguagacuugauuacccaagucuuuugccagagcgucua | |
| | uuaaccgcaaaaugucaucaauuuucucugauaagcuucugagaugcaccgccgug | |
| | guugauauuauguugaccauaucucugcccuccaucauaucugcauacaucaauac | |
| | aaguuccuggguaagacaugugaacuuguucucauucguaugcgucacaacgacaa | |
| | guucaggagugacaaagacuuggccuaccuuauggguuagcaucaccaauuuguu | |
| | ggccgcagaccuuguccuagcugccaccaucagaugccucuggaucuguuuuaua | |
| | uggagccaugcaaacuuggcuguggaccauuuugagugaaaccagaaugccggau | |
| | ccguacgaaugcugcugaacuccucugaccgggggacauuguuagaccaagaugac | |
| | cccagcaguuucuucucuauaugcguacacagccuugugaacaguucuccauaucu | |
| | cguguugugaauuuggaucuucuucucaaucuuccgaaauuuguugguugaaucu | |
| | gggaccucaauauuagccaguucuucuaaacaccugggguggagcacuccgguua | |
| | uucuggaauugugguuaagaguuugguguacugcccuuccgaguuuuaucauuc | |
| | ucucaguaucaggagaggccgauucaaguauuuuuuuccauugucggcugagaau | |
| | gagguggucgaagucacauucaucaggaagcgguagcccaguuaauuuccaguaa | |
| | uagaguaguuugugcuugaccaauggugaagacaggugugacucugguaggauaa | |
| | ucugaugcucugcccuuucaggaccggagcucgccauguccuacccguauuguua | |
| | accaugagcuguuuugccuuguaucucauugccacuuacauuuuuucuuaaucaa | |
| | gagacuauugacaaggcuuaaucugaucgcaugagcacuggcugauuguggucaa | |
| | cuggcaacauggaauucgagcacgcgccggcauucgguuugauucuugaugucgc | |
| | agaagauaggugauacaaugccaucuuuccaacuccuuuauaauugacucaacuag | |
| | ccagaccuggcuucucuaaccccgucaucuuugaggaucucaacuaguaacgggac | |
| | gauucugaauucuccgaagagaguauuagauauuucagcaaugcugagacaauag | |
| | gucuuauuagucuugaccacuuuaaaacaaguugauguuguguaugcugcuuugg | |
| | uacugcuugaacucacucgaguaaugcgacugcgggaugugcuaucgaauacugc | |
| | agacgcaggguuaagucuugcuuguacaccaucaagcauugucccgaauaccccuc | |
| | gcaaggugugguuucuauagaagauuaggggauauggaucuguauagacuccagu | |
| | aacacacgaguuggggcaucuugcugaagccuggcaagggauacuaccuggccgag | |
| | ugaaggcauugaauguauaaggacuaugaagaguggcuguuuuguugcugacugu | |
| | cauaggauauaauaacgcgggagagaaguaugaugacccucguugauacaagaaau | |
| | gagaugucccuacugugagaauucugccuucggcccccaugagugugacuguguu | |
| | gggcgguacagucaguaccgggucuucgccuaaggauguugacaccuugauagau | |
| | aagauagccugcuguaugcguuucccaccaaaccguccaggcuuauacgaagacuu | |
| | ggccauucgaaucugguagucuugcucaucugggcaugugucauuguaucgcuug | |
| | uauaucacauauuucccuuccuguacagugucacugggugaauuggguuuuaacc | |
| | cuccguagacugagaaccauacgcggcugucaauaaaagauccacccccuacuccu | |
| | ggguaguuggccacccaguccccgaauaauguugugacaucuagguccuuuucgu | |
| | gguacuggccgucgaacccuaaccucccauguaccauccgcguagggacagcugag | |
| | uuauaaucuucuuccucugucuccgugacuuucgagcacagcauaucacaacccag | |
| | gggaguugcacucacacugcaagacuuccgauuuugggugucguccagguugaug | |
| | gaacgcagaguagaaaagaauacccucccuguugcagauguccggagcacaccaag | |
| | ugcuaaauacugauaugaaugugagugaucucugcauccagacaauauuacauuau | |
| | ggguguagcaguaauggguagcacucaugucaaaugaggguauucgagugcaacc | |
| | ugauccuguaguaggcgccgggauaaaauucagauguucuugaaaugcagaggga | |
| | uagaaugaugugacaucacuagcaucaucuacaaugaguucuuugccuaucccccc | |
| | uauauaaucugggucauggauaggugccccccacccacuguuguuugcagcucca | |
| | uuaaucugauaagagagagauguuauugcguucauaauuguggucucaguauuua | |
| | acaaugccaacggagacucaagggccacuugcuuauauauccuaucuacuacaucu | |
| | ugauuggaaccaaguguagauguaaucuuuucuucugcccuggaaauccuagucg | |
| | guaugccuacaagaucgcuaggugugcuagcccccaugcuauauaaaagggaggcu | |
| | acagauauagccaaggucacuacuguuaagaauaagauugcaauccggaauaucaa | |
| | gcgccauguauuuuuugccucucuuucaucauucucuaacgcaacuuggcuaacg | |
| | gcgcgguccaugauugaggacuguugucggugaagcgguagaacggagguuguga | |
| | agccuggcucgcaauugaggggcggccucucuuaccguucuacccguauaucguc | |
| | cuuuggucaucuacaaccgguaguuuuuucuuaacucucugaacugacagacuacc | |
| | agaacuuucacacaaauuacuauuagggaaaccuucguuccucaucuguguucaca | |
| | uuuuuguaguggcucucaucugaucuagaguauuauucccaagccauaauaaggu | |
| | cuuuuguugcgccuuuugcuuguacauuagguagcaugcuagaaucaggcuaagu | |
| | auaccaaaaacaagagauaugauagucaaaacgauauagguaaugagagcagaugu | |
| | gcuagucaguuugacauugacuuugucuaguuuucuguugcuuuccucuaacuua | |
| | uucaaagcauuacugaucgaguuguugacauucccaagcucaguugagauaucaag | |
| | auugccuguuauuauuacuugagaaucuuguauugagauauucuucugauaaguu | |
| | acaucgaauuccccacugagccuuaaaguuaucccgccuaaggauaaaacauugca | |
| | ugauuguuuaucuauuagagacacggcuucuccauaguuuugcgauaugauaccc | |
| | gggggguuuacacaucuacauguugucaucuugcaguuggcgaugacugaaccuu | |
| | ugauagucauguaugguguaguaagugcgccuucggucuuugaguacauacaggc | |
| | cgacguauugccgcucaagcaggaauaaauaccaggggacauagggaacguuacua | |
| | uucuuguacaauauaaaucuaagucaguuucuauacaguaugaggugucaaguuc | |
| | uucuaucacagaaccgaccugugucaccacuuuugggacaagugccgaggcaaauc | |
| | cccugguugugcuuacggauaagguuuccaaguagguggcacgcauauuauuuag | |
| | guucccgacugaaggggcaguuaccuguauacccaagaguugagucugugagucg | |
| | uauagaauaggguuaccggugauuaagccgcuaccgauuaaugagcugaguugau | |
| | uguucccuacaccuaacuuagucaauaaguaauccauauuuccaccagcuagauug | |
| | uaaagugccugaauagucagcuuguuuaaagcaggugaagugauuugugguccga | |
| | auacuguagucaauucgguuagguacagguugagcucuacaccaacuugcugugc | |
| | aauuuugaugcagucuaauuccugagcuguuuuauuaaauuggucauuaacaaac | |
| | ugcugcaucuucccaacugccacugcuaguugcgauaauccgucagugaccucaug | |
| | cacagccucauugguugcggcaaugcucucuuuaagucggaggauguuggcagca | |
| | uuuuguuuggcuuguaucagagcugcggccgcuguuauuugugcggcaguugca | |
| | accccaagagccacaccgccaauaauggcgccuauaaggcgccccugucuccccccu | |
| | ccagauguagucacagacucuuguauccuacggauagagucaccaagggggguga | |
| | gcaaaguggucaauguccuguuguaugcauccaagggggcuuucgcacaugccuc | |
| | cuuauccuugggcagauucgggaggagcuuaacuaugauugauccugucugggau | |
| | gagguguauauguugacggcuuugucuccuguaaccacaauuccugcagcugcaa | |
| | gaggccugccaucaauggaguuugccggacagaugcaacucaguaccagcgcaacc | |
| | cggauagucagcaucauaggugcuggguucuugguagaaggucuggagcccaucu | |
| | ugcaccuggagggcgccaaccgggauccagaaucuucuacccguguuuuuucuaac | |
| | uugauagacagguaaacuaacuguaaauaaucaagacagauuaguuuuuuguguc | |
| | augaugaucugggugagugggcggagcgcaaucucagagacgcagcuuauuucuu | |
| | aaaaggauuguauuuggcaagggugugccccuucuccagcuuaguagagguaacc | |
| | ucguggucggcggucacugcgacagcgcguuggguaccugcuuggauaaugauuu | |
| | uaacgcuccgcaggcacgcgguuugacuccagaguaucuuggccaccugaggagaa | |
| | gcauuugcuaugggauagcaggcugucccacugcuagagaagaaaggugccaaaag | |
| | cuuaguccgugcaccucuugcuuuuaccaacacggaaggcccgagcacaucacuga | |
| | gcccgacagauagaucaaggcuccuuauuuucuuuuccagcuugucaaaugucacu | |
| | uucuuccccuuccuaucuacaguggucauaaguccaauaugcaagaagagguuagc | |
| | auaguauccgcugucagacuuagacagagauuuaaccaaaggacuccucgggucua | |
| | ccuccacauuaauagugacauugagcgcaagauuguacagacucgagccagaaacc | |
| | uucaauacugcagcugggaucuuguagacaucccucuucgguaccacagucaagga | |
| | gacaaaguucaccuuguauucuaggguuccacucccgggaaucuucucuggcgcu | |
| | uucacgugcuugacugcauucacugaugaguauuuguuugccacaacccuacagc | |
| | uuugcagcacuuggggugccugcacuacugagaaaaccauucucucaguauuagu | |
| | ugcacucuucuugcaugugacuaucauagugagacaggcccuugccagcucaauaa | |
| | ggucuccgguauuugggacgcuuccuaggcagagcaucgcagcggaaaguaacuc | |
| | gcgcuuggguuuaucaucgaucaugccgacgguggcuucuucauucccaacuuga | |
| | aagaugaauccauagguggugaugaauacugaguccuccuuacuaucaguccacaa | |
| | gucaaggcgcuggauccuauauugcggggcgauuugcuucuucccaucuccugug | |
| | ucuuguaggacgaucggaaaugcuaacagguugcuagaagaaugggcagaaucaaa | |
| | guacagcccaauuguccuagaugaguccaucuuggcacaauuggggcacuccaauu | |
| | cuacccguauuuuuucuuaaucuuaaauguagcuagauuaauuacgguuacgcga | |
| | ucauucaguggggcugaggaagcgagagaggauugccgcuuggagaguuggaccu | |
| | uggguccgcggaucauuaggaagcauucagauaggacaucacgcggucgauuguc | |
| | acggcucugauccgaaaggcgugcagcaggaugcacagcuugaucuuggucuugu | |
| | agaaaucuggcuccuccagagaggacuucugaggcacugucucgcuauugaaguu | |
| | cagggccugcaucagcucgucgaucacggccagcauauucugauccagaaagaucu | |
| | gccucuugggguccaucagcagcuuggcguucauggucuugaacuccaccugaua | |
| | caucuucagguccucguagaugcuagacaggcacagggccaucauaaagcuugucu | |
| | uucuagaggccaggcaggagccguuggugaugaagcuugucucccggcuguucag | |
| | acaggacucguucuuggucagcuccaguggcaggcaggccuccacuguggagguc | |
| | uuauccuuugugauguccucguggucgaucuccucgcugguacaaggguaaaacu | |
| | ccagugucuggcgggccuucugcagcauauuggacacggcccucagcagguucug | |
| | gcuguggugcaggcaggggaacaugccugggucagggguugccacuggcagauuc | |
| | cuagauccuccuccuccuccgccgcugcaaggcacagaggcccacucggaccagga | |
| | gcuagaauaguagcgguccugggcccucacgcugauagaggcguucuuucuacag | |
| | aucacgguggcagaugucuuaucggugaacacucuguccuucuucucccgcuugg | |
| | acuugcccugcaccugcacgcaaaaggucagagagaaauaggaguguggugugcuc | |
| | caggugucaggguacucccaggacaccuccaccugucugcuauucuucagaggcuu | |
| | cagcugcagguucuuagggggaucgggcuugaugaugucccggauaaagaaggag | |
| | cugguauaauucucguacuucagcuugugcacggcauccaccaucacuucgauagg | |
| | cagggacuccucugcugcuggacaggcagaguccuccuggcacuccacggaauacu | |
| | cguacuccuuguuaucgccgcgcacccucucggcgcucaggguggcugcuccgca | |
| | ugucacucccugugggucagaggauccccugcuagacuucacagaaaaugucagau | |
| | cgguggagauuguggucagccaccaacaggugaaccggccgcuauaauucuuggc | |
| | cucgcaccgcaggaaggucuuguucuuuggcuccuucugauccuucaggaugucu | |
| | guggaccagaugccauccuccuucuugugcagcagcagcagggaguggcucagcac | |
| | cucuccucccuugugacagguguacuggccggcgucgccgaacuccuucaccugga | |
| | uugucagggucuugccgcuucccagcaccucggagcucugauccaguguccaggu | |
| | gaugccauccuccucgggugugucgcaggucagcaccaccaucucgccaggggcau | |
| | cgggauaccaguccagcuccaccacguacacauccuuuuucaguucccagauugcg | |
| | accagaggugaggccagaaacaccagggagaaccaugagaugaccagcugcugaug | |
| | gcacaugguggcgguucuacccguauuuuuucuaaccgcgggggggaacucggug | |
| | cagauuccgugugaugccgaguggacagggacccgcuacguguggcaguaguaau | |
| | uagccauuuagagcaaggcgcuugauuuuccugauuuccucgaucgacccggcug | |
| | caucuaacuugcuuaggagcuuggcugaagaacucgggugcauugggcgugacau | |
| | gaucaaugcacggacaguguccuuuuccacuccuauaucaggcccgcaugcagugg | |
| | cggguuuaaucaauucagauggauguggcacugguugcgaaaguuuauuaagugc | |
| | cauuucgccuccuugugucacauagggagaggggucuccagggccugaaacuaaaa | |
| | ccgggugagaucgggcaacugcccguagaucacucagagaugaaauguuggcacaa | |
| | ccgggauccagaaucuucaucauucccaaguuggcuuccaugacugcaacagaugu | |
| | uuucagcuguuggauuucggaccgcaucauagggauggaggaugucuguuucaag | |
| | acaagaucuagcugauagucaaccuuacuuacucucugugauaucgccuccaucau | |
| | agacaucaucgcuugcacaaagucuacagguggcuggacaugauccgcagauacaa | |
| | ggguauugucuuggcucugccuugaucggagagcaugagggguugcaccagcuga | |
| | uaguugugacuccucccauuguccaugauaugcuguguucacgucugugcccugg | |
| | uuuccaggggcggccuugacuugguucugcggucuuuccugacuguuuccgcgac | |
| | uggguugacuccccugcuguugagucggacguuggugauuccccucuugggggc | |
| | ucgaccaugggcccuuuuuagcauuggacgauuuauugcugagcuugucaagcau | |
| | caacagcagagaguugcuugcuccgguccugagcugugugucgacggcuucgucu | |
| | guggccuggguggggggcuggucggcggauguggccggcgggcugucaugcggg | |
| | gucguuugcucggguguggaugguuguuugucagaucuguccugucgaucgggg | |
| | uugucuugacuggccgguggcuggaugcucccaugcuucucccaugcugcgcuca | |
| | gcaccuuggucuugccuugugggauugcacuccuuccaacagucucugcugguuu | |
| | acccugggcuguaauuauguugucaaugacaguuccacuugucucaaauagcucg | |
| | ucgaucucugcaucuguaaagguggccauguuuguccuggucuaucagguagagg | |
| | agagagcagagacucgggagacuugcccugaucucugaauaucccucuucuacccg | |
| | uacuuuuuucuaauucccucggcucuguuuugauuguuagugagccgcauugugc | |
| | cugugguaucuagggcguacguaguuguacagcagggggagggaggaaagagggg | |
| | gauguuuguuugaggguguggucauauagagccgcaaaucgaggggucgacuacg | |
| | ggugagggcauugggauguuuuuguggaagcaggcuggguuuuguccaucaaua | |
| | cccccagucggugucguuaucuugggauggcccaggaguugggggaggccccgau | |
| | uggggagugcccugugcagaguuuggcgccucccucaugcuauuugcuaccgcuc | |
| | ucaucagauccaggaauugggucuccccauccccggcuucugguugcccuugcga | |
| | ucuauucgauccgccuuguagagcuugggacccccccucgcuaagcccagugagga | |
| | cuccgacuuguugaguaggcaugucuaugcugcuggucuccucggagacccguug | |
| | ggcagcagcugccaggccccuccuugcugcugggguuagcuuuagcucggcagcc | |
| | auauccucguuaaugcuacuucccugagccugagcguacucuacuccaagucucca | |
| | gaaugaugugcucauaaagucccuggcaaauugguauuucccaguaccuuuaucu | |
| | aggacugaugccauacccauggcaaaggaguaaaguugugcauacucggcaggcgc | |
| | aaagcucaucuggucacuaucaccaaguaaugucauguacggcgcauuaucuccuu | |
| | ucauccgauacaaacgcaugagcugcuucaucuucuggaugucgccugagaggcua | |
| | cuaagugcaagggcugaugucuugguguugauuccguacuugagugucaagaaga | |
| | augcaguaagcccgguauuccugauguaugagucuacguccccuaccagguuauaa | |
| | uaaguagagguaccaccugccguguugcggccucucuugagcucgcuaaccaaaaa | |
| | gaugcggacugcaagagacugucugaucgugaguuggauugugcuccugcauacg | |
| | ggguagaggauguauuucuuuuggacccugccuugcugcauauacuuauugauuc | |
| | gccuuguuuccgacucaucugcagucucauacgcagucauggcuuuugcuacugu | |
| | gacccauacuugagccuggauagagaggauccucuccaggguaucggugaugucu | |
| | ucuggugcaucaucuucggccccggcugugacgaacgggguuccguugcugcaug | |
| | cccgagggagagauccugcuaucaucgcaaaucucugugcucucucuucagacacu | |
| | ccacuccuauuguugaacuggggcgugccguuggcaaagccaucaaucucaagcac | |
| | ggccaauguggcuucauucuguuucccugcaagggcaacaugguuccucauuacc | |
| | ugugagugggagcauaaaagagauaugagagcaccuugccugagugguuuguugg | |
| | caucuucgcuaacagcaauccggaggcagaauaccacaaagcuccaucuaucuucu | |
| | gggucaucacuguuaagagugaauaccgggacgucuacuuuuaagguacucccuu | |
| | uuucucccccuccaugagcuccauuggggcgagucugagccgcgaggagcuguuc | |
| | guacucaucaaauacggaagacauguuggcagaaggcuuucucgaguuugugcuu | |
| | cgggcucgcacucgagauucacaccuucuacccgugcgacuucaauugcuccuucg | |
| | ccuuuuaucguaacucacggauucucuguuuggu | |
| Exemplary | accaaacaaagauuuggugaaugacgagacuacacucaagaauaauugugcgcaac | 60 |
| NDV-huIL-12 | cuuuuuuuaagacauuuauuugaguucgaauucgaguccuaaggagucaggguuc | |
| genomic RNA | aacuuuuuucuaacauaauaugauuaaaucaacaagaauacaauuggccaaaaaag | |
| sequence (non- | gagccuauuaauaugugauuuucguuaagagucacaguuacuguaauauccuuug | |
| codon- | acugcauugccuauaguuuucauguagaauuuuuguugagcacgagucaaguaca | |
| optimized) | guacagaagugucuguaaacauuucuuugaguuugguaauaccuaggacagccuu | |
| (Version 3) | caaauauuuagggcagguacuaugcuucaaguauguccuuagugggauaaggucc | |
| | uccauggagaucaugccuuuaagcacuaggcugauuauaucgccuauuuuauuga | |
| | gauuuucgacucuaagaccuaguauuguaaccucuaggaucugucucgugcacug | |
| | uuuaagugauguccucuuuuucccgucaguagagagauuguaagggguaaauaga | |
| | aauacugugucagcgagaucacccucagcuuccauauauaucacagaccggauaac | |
| | ugugucaaugugacuagcgaugaucuggguuaucugaguuauguucgcuagcgu | |
| | gcucaccagacucuccgcacagaauggacggacgggcugucccccggcuucaauca | |
| | gcgcagugucaauauucuuccucaaguacuuuauuaaucuugguaaaggacugga | |
| | uaggaugucugucacacgcugccgcugugaggugaauaaccuggucagugugauc | |
| | ucaucugauuuagacaaaagcguaccgugccgcugcaccagaguuuucgccauccu | |
| | caccaccucauguacaaauguaggcccgcccagguaacccaugacaaauaccaggua | |
| | acacuccauaucuccucgacaugcauaaccauuagagagaauauauccuuuugugg | |
| | aacacggagcaaacaaguucaugaguagaugaaaguaguaucccauugcauacaac | |
| | acuuugaugauuacuaccccgcccucccuuacagaaugcauggcaaucagagauaa | |
| | auugauagcuaguugaucuaguaagcuuugauuggacccuggaggaauuucaaug | |
| | ucacaaugcagcaaugauacagaucuguagggcacugcagauguaauauaccccac | |
| | uacuuuaucugaggucaggucgcuuuccucuguauuuucucuccauaauggacgg | |
| | aacucuuggacaaauccaucuuugcauguuaccuccgccuguagauuccuauaaac | |
| | aaccgaauucaaaaacugaguuggggucggcccgaaaugucguugcgggggguuc | |
| | aucucauuugaaaagagcguauuguaauagauaguuucaugugguacaugcaguu | |
| | cgagaagacucaugauggcuccgcucccuucagcuaaguauaaggaguucccgugu | |
| | cuugcacaucuuaccucggguacagaaaggagaugagaugccuuauaccaagagga | |
| | agaugcagucccuaucccucugaacaaguaucguacuaaguagucuuccuccggau | |
| | uuggagaugugaguucaggaugaaucugacuaagugugaaugcgucauaccuugc | |
| | uggagcacucaaaucuaacucuugcaaaaaugccgcagguugucgggugaaugga | |
| | ucuuucacucgagcaccaauaucuugcacagaagggaacucuaauaauggcucuug | |
| | ggggaacaacaacgccaggauaguaucccuguccucccuuucccugaucaaauuga | |
| | ggcucuuccgagacauguaguacagauuagcugggaauguaauuauguuaggaga | |
| | caugauagagcucacuugaucggggcuaaguaaugguuucacagcauccgacagua | |
| | aauacucagugaguauugaacauuucucuucugcaguuaagccucuuauuuucgg | |
| | gauuucucuuguuguagcaaagaguaccguguacagacagcauaaccgggauauca | |
| | gcugaagcaucuucucauucagguuaucaucuaagacagaugggaacagcagauca | |
| | uacuuauuuccugaauauaagccggagaucacacgucgggugcaagauacuaauag | |
| | uuuugcagacauuucgauaaaauccguaucugcaaguuggugugauccgucaugg | |
| | uugaccaggcccacugcauguaaccuugaaugaaugacgggaugagauauuguag | |
| | cugcaauguuggaaaguagaauuccuggcauauucuuguauaaaucacccauauau | |
| | aagacaauauuguccaggccucuuacucucagguaauagaguugguaagaacaguc | |
| | gaggagcacuucaagugcugcauauucaaauaggcggaccacaucugaauucugag | |
| | cuucacugauccaauuucggguauugucauacacuauuauggcgucauucuuuau | |
| | ggagguaucuucaucauaagaaaccacagacuggccaaucaacuucccgcuggaua | |
| | uugaaagaauguucauuagcucuaucgugggauaugacuccagauuaagcucaua | |
| | acucuugaagauagcuaagucaagucucgcaaagucucccuccgauacagggcuag | |
| | gaucauacauaaacuuauuugaggucacuguccucaguuccgguaccaccccaagu | |
| | agcucgaaaggaaccgcaacaggugcuucucugauacagcaacuaaauuuacuaug | |
| | uaggugcagugugaucucaucauauguccugguuguugucauuggaaagaucgau | |
| | ucgauuagagauaaacccaagagcaugaucuguugguaaaccacauuccccucuuu | |
| | gacuccuucuucagugaacagccuuugagaaucauuggauaugugaauguaaggu | |
| | gacacccuguagagagaugcaggggugaaugucaucugaguuauaccaucaucua | |
| | gucuauguugaagauucccagccguggguaaaggggacaguaaccgaagauacuc | |
| | uaaguuuacauuacaccgagauuuugcaaucguaagagcagcaguccaauuuacuu | |
| | cauuauccccauaagcccagaucaacacggaugaugcccuuagggcagccuuuaca | |
| | uguggcgacauaugagcuauuuuugcaagugaggcagcucuccucuccugugucu | |
| | uugacccgagauaugguacccucaucggaggauucuugcuggugucaucggucaa | |
| | uucuauauugcuuggaagauggaaccaaguaaauuguucaucuccgcugucacau | |
| | cuuguacacccuccgcuuacacuaagaaucucacccucuacgaguucuaucguauc | |
| | aggauuagauacacccaguauuuuccugccucccgucaaaggugaccagcuucuau | |
| | uccgugcauagucugccagugucagagaacacauauuagaagagacuaaggggug | |
| | guuggaucuacuggaggaaaaaacaucgucucuaaacagcauugcaugcaugcuag | |
| | aauaauugacuauccgcaucagccucuugaugccuaauggccuccuaguaagcgca | |
| | aucuuaauuacgguguuuguugugucaacaagcccuugaauuugcuuucuccuac | |
| | cuacagagcuugccuccaugauggcaugcgcaacgcggggaugaaucaccucuuga | |
| | uuaagcaagaauucagccaaugccuucucuucugccucauuauccucugugugcac | |
| | uccagacaauaagggauuugaacaaguuucaaauaggacucuuugcguauguuuc | |
| | uuaagaacaauauuugggcuugcaacagucucaaaauugaaagaguaugggucgu | |
| | ugcacagacuggcccaaucuccauucccaggcggccuaguuaagauauuagucaua | |
| | auguuaggacucaguaaucccacugcuucuagucgcuugaucucugcaaaagcagu | |
| | aguccccgggucaccgauauuucuaguguagagccuugaguauugaagguuacuc | |
| | agucccccuaauugggcaggagucagaacauaugagugcacaaaagagauguccuc | |
| | aauccacgacugauuaagaucggggugcgaauuguuggugauggagaacucagag | |
| | ucaaaguaugucugcacacaacucauuauauaguuuaaauaguaacagaagucuuu | |
| | gggaagcccguucucgcauagccgugcuacaguagaggcaauguuggcacaggaca | |
| | uuacgguguuuucacugagaucaccugacacuagcacuaauuuagaugaauuuuu | |
| | gaggacuugacugaggauugcuccaucuuugaagauucguuugcuguauaugaag | |
| | aaugugucugaccugaugguuucacgauccuucaaauuauggccaaucaaaugau | |
| | ugacaugaauuaauuccuugaagaaauuaucacuggcuugaugcaacugugucaac | |
| | accaucuccggagagucgucugaucuuaccucucucguuacugcuauuacuugau | |
| | uaucacccuguaccauacaggcaacacgacaaugcgaucuagcugcagcaaguugg | |
| | auugcagcaauugagaucauuguccauagcuucuggcauaauccuucgauaccccc | |
| | ucuggcacugacaauauauaugucaucauuagggacucuugagaggucacaguca | |
| | guagggucacuuggaggauugaaagggucuccuacgaacaucguaguguccauca | |
| | gucuuaggugaauccauucgaagaagugagguaggcccaucaacugauugauggc | |
| | augagcgaacaauuugauugucugauaucuccaauuaagacaguacuuuugcagg | |
| | ucaguuguuaugaagguugcaacucuccgacgguucuugcuuuucggaucaugau | |
| | ugcgguuugaagauacucuuucuuuacagucagugauacguuucuuauugcuguu | |
| | aaaagacaguugacucaucgcuagcauacucuuggucaaggauaugcuauccugaa | |
| | ugacuccauuucccugaaagaaaggugcaaucugaucggcuaggaucccuuccgcc | |
| | aucaccugacaguuccuuaacuucuuugucagcuuagcgaagauccguccauuaac | |
| | uuucacuuccuucuccuugagcgaguaugauacugccacauugucaucucuaagg | |
| | uacucaagggucgucagauauuccaucucuuuauauggaucaaaaucauuugacuc | |
| | uaaaaacucuaucaagaggcgauuagucgaaguugcuucuuuuacauguuucuuc | |
| | uggucuucggagagaagguuccgccuaaacgaggcaagccaauuaucguuggggu | |
| | gugcgauugccuugucuuuuaggaacaugcucagguuggugacagggucauauuc | |
| | uauacauggcucaaauucaagugcagauaaacucuuauacucucucaacaugauau | |
| | cgugugaaaucucugcugaaucugcauguaguugcccaaugaccuucccauauau | |
| | uguauccacuuugacucgcggccacacaccugcauucuucuuucuguacccguuga | |
| | ugauuguucccuugaagaaagacaguaccugaaggaucauaucaaagucuaccauu | |
| | uucggugcgcacauuuggcuccugacugccuuugcugcaauacgggacucaagca | |
| | gugggugaccccacagacgcaacagacacaacaucucagcugcuugauucuguucu | |
| | aaaccagagaauacaguagcgauugcaugagucacggauucugcuauaucauuggg | |
| | gaggaggccaauuagaaugucuuuaagcuccugcagguugaaugcgaagaaaucu | |
| | ccugcaaauguaccugacggcucgaguagcuggacagcuccguaugcaaaucccuc | |
| | cauuagugauacaacaucguagacuugauuacccaagucuuuugccagagcgucua | |
| | uuaaccgcaaaaugucaucaauuuucucugauaagcuucugagaugcaccgccgug | |
| | guugauauuauguugaccauaucucugcccuccaucauaucugcauacaucaauac | |
| | aaguuccuggguaagacaugugaacuuguucucauucguaugcgucacaacgacaa | |
| | guucaggagugacaaagacuuggccuaccuuauggguuagcaucaccaauuuguu | |
| | ggccgcagaccuuguccuagcugccaccaucagaugccucuggaucuguuuuaua | |
| | uggagccaugcaaacuuggcuguggaccauuuugagugaaaccagaaugccggau | |
| | ccguacgaaugcugcugaacuccucugaccgggggacauuguuagaccaagaugac | |
| | cccagcaguuucuucucuauaugcguacacagccuugugaacaguucuccauaucu | |
| | cguguugugaauuuggaucuucuucucaaucuuccgaaauuuguugguugaaucu | |
| | gggaccucaauauuagccaguucuucuaaacaccugggguggagcacuccgguua | |
| | uucuggaauugugguuaagaguuugguguacugcccuuccgaguuuuaucauuc | |
| | ucucaguaucaggagaggccgauucaaguauuuuuuuccauugucggcugagaau | |
| | gagguggucgaagucacauucaucaggaagcgguagcccaguuaauuuccaguaa | |
| | uagaguaguuugugcuugaccaauggugaagacaggugugacucugguaggauaa | |
| | ucugaugcucugcccuuucaggaccggagcucgccauguccuacccguauuguua | |
| | accaugagcuguuuugccuuguaucucauugccacuuacauuuuuucuuaaucaa | |
| | gagacuauugacaaggcuuaaucugaucgcaugagcacuggcugauuguggucaa | |
| | cuggcaacauggaauucgagcacgcgccggcauucgguuugauucuugaugucgc | |
| | agaagauaggugauacaaugccaucuuuccaacuccuuuauaauugacucaacuag | |
| | ccagaccuggcuucucuaaccccgucaucuuugaggaucucaacuaguaacgggac | |
| | gauucugaauucuccgaagagaguauuagauauuucagcaaugcugagacaauag | |
| | gucuuauuagucuugaccacuuuaaaacaaguugauguuguguaugcugcuuugg | |
| | uacugcuugaacucacucgaguaaugcgacugcgggaugugcuaucgaauacugc | |
| | agacgcaggguuaagucuugcuuguacaccaucaagcauugucccgaauaccccuc | |
| | gcaaggugugguuucuauagaagauuaggggauauggaucuguauagacuccagu | |
| | aacacacgaguuggggcaucuugcugaagccuggcaagggauacuaccuggccgag | |
| | ugaaggcauugaauguauaaggacuaugaagaguggcuguuuuguugcugacugu | |
| | cauaggauauaauaacgcgggagagaaguaugaugacccucguugauacaagaaau | |
| | gagaugucccuacugugagaauucugccuucggcccccaugagugugacuguguu | |
| | gggcgguacagucaguaccgggucuucgccuaaggauguugacaccuugauagau | |
| | aagauagccugcuguaugcguuucccaccaaaccguccaggcuuauacgaagacuu | |
| | ggccauucgaaucugguagucuugcucaucugggcaugugucauuguaucgcuug | |
| | uauaucacauauuucccuuccuguacagugucacugggugaauuggguuuuaacc | |
| | cuccguagacugagaaccauacgcggcugucaauaaaagauccacccccuacuccu | |
| | ggguaguuggccacccaguccccgaauaauguugugacaucuagguccuuuucgu | |
| | gguacuggccgucgaacccuaaccucccauguaccauccgcguagggacagcugag | |
| | uuauaaucuucuuccucugucuccgugacuuucgagcacagcauaucacaacccag | |
| | gggaguugcacucacacugcaagacuuccgauuuugggugucguccagguugaug | |
| | gaacgcagaguagaaaagaauacccucccuguugcagauguccggagcacaccaag | |
| | ugcuaaauacugauaugaaugugagugaucucugcauccagacaauauuacauuau | |
| | ggguguagcaguaauggguagcacucaugucaaaugaggguauucgagugcaacc | |
| | ugauccuguaguaggcgccgggauaaaauucagauguucuugaaaugcagaggga | |
| | uagaaugaugugacaucacuagcaucaucuacaaugaguucuuugccuaucccccc | |
| | uauauaaucugggucauggauaggugccccccacccacuguuguuugcagcucca | |
| | uuaaucugauaagagagagauguuauugcguucauaauuguggucucaguauuua | |
| | acaaugccaacggagacucaagggccacuugcuuauauauccuaucuacuacaucu | |
| | ugauuggaaccaaguguagauguaaucuuuucuucugcccuggaaauccuagucg | |
| | guaugccuacaagaucgcuaggugugcuagcccccaugcuauauaaaagggaggcu | |
| | acagauauagccaaggucacuacuguuaagaauaagauugcaauccggaauaucaa | |
| | gcgccauguauuuuuugccucucuuucaucauucucuaacgcaacuuggcuaacg | |
| | gcgcgguccaugauugaggacuguugucggugaagcgguagaacggagguuguga | |
| | agccuggcucgcaauugaggggcggccucucuuaccguucuacccguauaucguc | |
| | cuuuggucaucuacaaccgguaguuuuuucuuaacucucugaacugacagacuacc | |
| | agaacuuucacacaaauuacuauuagggaaaccuucguuccucaucuguguucaca | |
| | uuuuuguaguggcucucaucugaucuagaguauuauucccaagccauaauaaggu | |
| | cuuuuguugcgccuuuugcuuguacauuagguagcaugcuagaaucaggcuaagu | |
| | auaccaaaaacaagagauaugauagucaaaacgauauagguaaugagagcagaugu | |
| | gcuagucaguuugacauugacuuugucuaguuuucuguugcuuuccucuaacuua | |
| | uucaaagcauuacugaucgaguuguugacauucccaagcucaguugagauaucaag | |
| | auugccuguuauuauuacuugagaaucuuguauugagauauucuucugauaaguu | |
| | acaucgaauuccccacugagccuuaaaguuaucccgccuaaggauaaaacauugca | |
| | ugauuguuuaucuauuagagacacggcuucuccauaguuuugcgauaugauaccc | |
| | gggggguuuacacaucuacauguugucaucuugcaguuggcgaugacugaaccuu | |
| | ugauagucauguaugguguaguaagugcgccuucggucuuugaguacauacaggc | |
| | cgacguauugccgcucaagcaggaauaaauaccaggggacauagggaacguuacua | |
| | uucuuguacaauauaaaucuaagucaguuucuauacaguaugaggugucaaguuc | |
| | uucuaucacagaaccgaccugugucaccacuuuugggacaagugccgaggcaaauc | |
| | cccugguugugcuuacggauaagguuuccaaguagguggcacgcauauuauuuag | |
| | guucccgacugaaggggcaguuaccuguauacccaagaguugagucugugagucg | |
| | uauagaauaggguuaccggugauuaagccgcuaccgauuaaugagcugaguugau | |
| | uguucccuacaccuaacuuagucaauaaguaauccauauuuccaccagcuagauug | |
| | uaaagugccugaauagucagcuuguuuaaagcaggugaagugauuugugguccga | |
| | auacuguagucaauucgguuagguacagguugagcucuacaccaacuugcugugc | |
| | aauuuugaugcagucuaauuccugagcuguuuuauuaaauuggucauuaacaaac | |
| | ugcugcaucuucccaacugccacugcuaguugcgauaauccgucagugaccucaug | |
| | cacagccucauugguugcggcaaugcucucuuuaagucggaggauguuggcagca | |
| | uuuuguuuggcuuguaucagagcugcggccgcuguuauuugugcggcaguugca | |
| | accccaagagccacaccgccaauaauggcgccuauaaggcgccccugucuccccccu | |
| | ccagauguagucacagacucuuguauccuacggauagagucaccaagggggguga | |
| | gcaaaguggucaauguccuguuguaugcauccaagggggcuuucgcacaugccuc | |
| | cuuauccuugggcagauucgggaggagcuuaacuaugauugauccugucugggau | |
| | gagguguauauguugacggcuuugucuccuguaaccacaauuccugcagcugcaa | |
| | gaggccugccaucaauggaguuugccggacagaugcaacucaguaccagcgcaacc | |
| | cggauagucagcaucauaggugcuggguucuugguagaaggucuggagcccaucu | |
| | ugcaccuggagggcgccaaccgggauccagaaucuucuacccguguuuuuucuaac | |
| | uugauagacagguaaacuaacuguaaauaaucaagacagauuaguuuuuuguguc | |
| | augaugaucugggugagugggcggagcgcaaucucagagacgcagcuuauuucuu | |
| | aaaaggauuguauuuggcaagggugugccccuucuccagcuuaguagagguaacc | |
| | ucguggucggcggucacugcgacagcgcguuggguaccugcuuggauaaugauuu | |
| | uaacgcuccgcaggcacgcgguuugacuccagaguaucuuggccaccugaggagaa | |
| | gcauuugcuaugggauagcaggcugucccacugcuagagaagaaaggugccaaaag | |
| | cuuaguccgugcaccucuugcuuuuaccaacacggaaggcccgagcacaucacuga | |
| | gcccgacagauagaucaaggcuccuuauuuucuuuuccagcuugucaaaugucacu | |
| | uucuuccccuuccuaucuacaguggucauaaguccaauaugcaagaagagguuagc | |
| | auaguauccgcugucagacuuagacagagauuuaaccaaaggacuccucgggucua | |
| | ccuccacauuaauagugacauugagcgcaagauuguacagacucgagccagaaacc | |
| | uucaauacugcagcugggaucuuguagacaucccucuucgguaccacagucaagga | |
| | gacaaaguucaccuuguauucuaggguuccacucccgggaaucuucucuggcgcu | |
| | uucacgugcuugacugcauucacugaugaguauuuguuugccacaacccuacagc | |
| | uuugcagcacuuggggugccugcacuacugagaaaaccauucucucaguauuagu | |
| | ugcacucuucuugcaugugacuaucauagugagacaggcccuugccagcucaauaa | |
| | ggucuccgguauuugggacgcuuccuaggcagagcaucgcagcggaaaguaacuc | |
| | gcgcuuggguuuaucaucgaucaugccgacgguggcuucuucauucccaacuuga | |
| | aagaugaauccauagguggugaugaauacugaguccuccuuacuaucaguccacaa | |
| | gucaaggcgcuggauccuauauugcggggcgauuugcuucuucccaucuccugug | |
| | ucuuguaggacgaucggaaaugcuaacagguugcuagaagaaugggcagaaucaaa | |
| | guacagcccaauuguccuagaugaguccaucuuggcacaauuggggcacuccaauu | |
| | cuacccguauuuuuucuuaaucuuaaauguagcuagauuaauuacgguuacgcga | |
| | ucauucaguggggcugaggaagcgagagaggauugccgcuuggagaguuggaccu | |
| | uggguccgcggaucauuaggaagcauucagauagcucaucacucuaucaauaguca | |
| | cugcccgaauucugaaagcaugaagaaguaugcagagcuugauuuuaguuuuaua | |
| | aaaauccgguucuucaagggaggauuuuuguggcacagucucacuguugaaauuc | |
| | agggccugcaucagcucaucaauaacugccagcauguuuugaucuagaaagaucug | |
| | ccucuuaggauccaucagaagcuuugcauucauggucuugaacuccaccugguaca | |
| | ucuucaagucuucauaaauacuacuaaggcacagggccaucauaaaagaggucuuu | |
| | cuggaggccaggcaacucccauuaguuaugaaagaggucucucuggaauuuaggc | |
| | aacucucauucuugguuaauuccaaugguaaacaggccuccacugugcugguuuu | |
| | aucuuuugugauaucuucaugaucaaucucuucagaagugcaaggguaaaauucu | |
| | agaguuugucuggccuucuggagcauguugcugacggcccucagcagguuuuggg | |
| | aguggugaaggcaugggaacauuccugggucuggaguggccacggggagguuucu | |
| | agauccgccgccaccgccaccacugcagggcacagaugcccauucgcuccaagauga | |
| | gcuauaguagcgguccugggcccgcacgcuaaugcuggcauuuuugcggcagaug | |
| | accguggcugaggucuuguccgugaagacucuaucuuucuuuucucucuugcucu | |
| | ugcccuggaccugaacgcagaaugucagggagaaguaggaauguggaguacucca | |
| | ggugucaggguacucccagcugaccuccaccugccgagaauucuuuaauggcuuca | |
| | gcugcaaguucuugggugggucagguuugaugaugucccugaugaagaagcugcu | |
| | gguguaguuuucauacuugagcuugugaacggcauccaccaugaccucaaugggc | |
| | agacucuccucagcagcugggcaggcacuguccuccuggcacuccacugaguacuc | |
| | auacuccuuguuguccccucugacucucucugcagagaguguagcagcuccgcacg | |
| | ucaccccuugggggucagaagagccucugcugcuuuugacacugaaugucaaauca | |
| | guacugauugucgucagccaccagcaggugaaacguccagaauaauucuuggccuc | |
| | gcaucuuagaaaggucuuauuuuuggguucuuucugguccuuuaaaauaucagug | |
| | gaccaaauuccaucuuccuuuuugugaagcagcaggagcgaauggcuuagaaccuc | |
| | gccuccuuugugacagguguacuggccagcaucuccaaacucuuugacuuggaug | |
| | gucaggguuuugccagagccuaagaccucacugcucugguccaagguccagguga | |
| | uaccaucuucuucaggggugucacaggugaggaccaccauuucuccaggggcaucc | |
| | ggauaccaauccaauucuacgacauaaacaucuuucuucaguucccauauggccac | |
| | gaggggagaugccagaaaaaccagggaaaaccaagagaugaccaacugcuggugac | |
| | acaugguggcgguucuacccguauuuuuucuaaccgcgggggggaacucggugca | |
| | gauuccgugugaugccgaguggacagggacccgcuacguguggcaguaguaauua | |
| | gccauuuagagcaaggcgcuugauuuuccugauuuccucgaucgacccggcugca | |
| | ucuaacuugcuuaggagcuuggcugaagaacucgggugcauugggcgugacauga | |
| | ucaaugcacggacaguguccuuuuccacuccuauaucaggcccgcaugcaguggcg | |
| | gguuuaaucaauucagauggauguggcacugguugcgaaaguuuauuaagugcca | |
| | uuucgccuccuugugucacauagggagaggggucuccagggccugaaacuaaaacc | |
| | gggugagaucgggcaacugcccguagaucacucagagaugaaauguuggcacaacc | |
| | gggauccagaaucuucaucauucccaaguuggcuuccaugacugcaacagauguuu | |
| | ucagcuguuggauuucggaccgcaucauagggauggaggaugucuguuucaagac | |
| | aagaucuagcugauagucaaccuuacuuacucucugugauaucgccuccaucauag | |
| | acaucaucgcuugcacaaagucuacagguggcuggacaugauccgcagauacaagg | |
| | guauugucuuggcucugccuugaucggagagcaugagggguugcaccagcugaua | |
| | guugugacuccucccauuguccaugauaugcuguguucacgucugugcccugguu | |
| | uccaggggcggccuugacuugguucugcggucuuuccugacuguuuccgcgacug | |
| | gguugacuccccugcuguugagucggacguuggugauuccccucuugggggcucg | |
| | accaugggcccuuuuuagcauuggacgauuuauugcugagcuugucaagcaucaa | |
| | cagcagagaguugcuugcuccgguccugagcugugugucgacggcuucgucugug | |
| | gccuggguggggggcuggucggcggauguggccggcgggcugucaugcgggguc | |
| | guuugcucggguguggaugguuguuugucagaucuguccugucgaucgggguug | |
| | ucuugacuggccgguggcuggaugcucccaugcuucucccaugcugcgcucagca | |
| | ccuuggucuugccuugugggauugcacuccuuccaacagucucugcugguuuacc | |
| | cugggcuguaauuauguugucaaugacaguuccacuugucucaaauagcucgucg | |
| | aucucugcaucuguaaagguggccauguuuguccuggucuaucagguagaggaga | |
| | gagcagagacucgggagacuugcccugaucucugaauaucccucuucuacccguac | |
| | uuuuuucuaauucccucggcucuguuuugauuguuagugagccgcauugugccu | |
| | gugguaucuagggcguacguaguuguacagcagggggagggaggaaagaggggga | |
| | uguuuguuugaggguguggucauauagagccgcaaaucgaggggucgacuacggg | |
| | ugagggcauugggauguuuuuguggaagcaggcuggguuuuguccaucaauaccc | |
| | ccagucggugucguuaucuugggauggcccaggaguugggggaggccccgauugg | |
| | ggagugcccugugcagaguuuggcgccucccucaugcuauuugcuaccgcucuca | |
| | ucagauccaggaauugggucuccccauccccggcuucugguugcccuugcgaucu | |
| | auucgauccgccuuguagagcuugggacccccccucgcuaagcccagugaggacuc | |
| | cgacuuguugaguaggcaugucuaugcugcuggucuccucggagacccguugggc | |
| | agcagcugccaggccccuccuugcugcugggguuagcuuuagcucggcagccaua | |
| | uccucguuaaugcuacuucccugagccugagcguacucuacuccaagucuccagaa | |
| | ugaugugcucauaaagucccuggcaaauugguauuucccaguaccuuuaucuagg | |
| | acugaugccauacccauggcaaaggaguaaaguugugcauacucggcaggcgcaaa | |
| | gcucaucuggucacuaucaccaaguaaugucauguacggcgcauuaucuccuuuca | |
| | uccgauacaaacgcaugagcugcuucaucuucuggaugucgccugagaggcuacua | |
| | agugcaagggcugaugucuugguguugauuccguacuugagugucaagaagaaug | |
| | caguaagcccgguauuccugauguaugagucuacguccccuaccagguuauaauaa | |
| | guagagguaccaccugccguguugcggccucucuugagcucgcuaaccaaaaagau | |
| | gcggacugcaagagacugucugaucgugaguuggauugugcuccugcauacgggg | |
| | uagaggauguauuucuuuuggacccugccuugcugcauauacuuauugauucgcc | |
| | uuguuuccgacucaucugcagucucauacgcagucauggcuuuugcuacugugac | |
| | ccauacuugagccuggauagagaggauccucuccaggguaucggugaugucuucu | |
| | ggugcaucaucuucggccccggcugugacgaacgggguuccguugcugcaugccc | |
| | gagggagagauccugcuaucaucgcaaaucucugugcucucucuucagacacucca | |
| | cuccuauuguugaacuggggcgugccguuggcaaagccaucaaucucaagcacggc | |
| | caauguggcuucauucuguuucccugcaagggcaacaugguuccucauuaccugu | |
| | gagugggagcauaaaagagauaugagagcaccuugccugagugguuuguuggcau | |
| | cuucgcuaacagcaauccggaggcagaauaccacaaagcuccaucuaucuucuggg | |
| | ucaucacuguuaagagugaauaccgggacgucuacuuuuaagguacucccuuuuu | |
| | cucccccuccaugagcuccauuggggcgagucugagccgcgaggagcuguucgua | |
| | cucaucaaauacggaagacauguuggcagaaggcuuucucgaguuugugcuucgg | |
| | gcucgcacucgagauucacaccuucuacccgugcgacuucaauugcuccuucgccu | |
| | uuuaucguaacucacggauucucuguuuggu | |

TABLE 10
Exemplary plasmid sequences encoding chimeric NDVs (nucleic acid)

| | | SEQ |
|---|---|---|
| Name | Sequence | ID NO. |
| Nucleic acid | tcagctcactcaaaggcggtaatacggttatccacagaatcaggggataacgcaggaaag | 31 |
| sequence of the | aacatgtgagcaaaaggccagcaaaaggccaggaaccgtaaaaaggccgcgttgctggc | |
| pT7NDV-LS- | gtttttccataggctccgcccccctgacgagcatcacaaaaatcgacgctcaagtcagagg | |
| L289A plasmid | tggcgaaacccgacaggactataaagataccaggcgtttccccctggaagctccctcgtg | |
| containing a | cgctctcctgttccgaccctgccgcttaccggatacctgtccgcctttctcccttcggga | |
| human IL-12 | agcgtggcgctttctcatagctcacgctgtaggtatctcagttcggtgtaggtcgttcgc | |
| transgene (gene | tccaagctgggctgtgtgcacgaaccccccgttcagcccgaccgctgcgccttatccggt | |
| open reading | aactatcgtcttgagtccaacccggtaagacacgacttatcgccactggcagcagccact | |
| frame in bold) | ggtaacaggattagcagagcgaggtatgtaggcggtgctacagagttcttgaagtggtgg | |
| (Version 1) | cctaactacggctacactagaagaacagtatttggtatctgcgctctgctgaagccagtt | |
| GCC = codon for | accttcggaaaaagagttggtagctcttgatccggcaaacaaaccaccgctggtagcggt | |
| L289A mutation | ggtttttttgtttgcaagcagcagattacgcgcagaaaaaaaggatctcaagaagatcct | |
| | ttgatcttttctacggggtctgacgctcagtggaacgaaaactcacgttaagggattttg | |
| | gtcatgagattatcaaaaaggatcttcacctagatccttttaaattaaaaatgaagtttt | |
| | aaatcaatctaaagtatatatgagtaaacttggtctgacagttaccaatgcttaatcagt | |
| | gaggcacctatctcagcgatctgtctatttcgttcatccatagttgcctgactccccgtc | |
| | gtgtagataactacgatacgggagggcttaccatctggccccagtgctgcaatgataccg | |
| | cgagacccacgctcaccggctccagatttatcagcaataaaccagccagccggaagggcc | |
| | gagcgcagaagtggtcctgcaactttatccgcctccatccagtctattaattgttgccgg | |
| | gaagctagagtaagtagttcgccagttaatagtttgcgcaacgttgttgccattgctaca | |
| | ggcatcgtggtgtcacgctcgtcgtttggtatggcttcattcagctccggttcccaacga | |
| | tcaaggcgagttacatgatcccccatgttgtgcaaaaaagcggttagctccttcggtcct | |
| | ccgatcgttgtcagaagtaagttggccgcagtgttatcactcatggttatggcagcactg | |
| | cataattctcttactgtcatgccatccgtaagatgcttttctgtgactggtgagtactca | |
| | accaagtcattctgagaatagtgtatgcggcgaccgagttgctcttgcccggcgtcaata | |
| | cgggataataccgcgccacatagcagaactttaaaagtgctcatcattggaaaacgttct | |
| | tcggggcgaaaactctcaaggatcttaccgctgttgagatccagttcgatgtaacccact | |
| | cgtgcacccaactgatcttcagcatcttttactttcaccagcgtttctgggtgagcaaaa | |
| | acaggaaggcaaaatgccgcaaaaaagggaataagggcgacacggaaatgttgaatactc | |
| | atactcttcctttttcaatattattgaagcatttatcagggttattgtctcatgagcgga | |
| | tacatatttgaatgtatttagaaaaataaacaaataggggttccgcgcacatttccccga | |
| | aaagtgccacctgacgtctaagaaaccattattatcatgacattaacctataaaaatagg | |
| | cgtatcacgaggccctttcgtctcgcgcgtttcggtgatgacggtgaaaacctctgacac | |
| | atgcagctcccggagacggtcacagcttgtctgtaagcggatgccgggagcagacaagcc | |
| | cgtcagggcgcgtcagcgggtgttggcgggtgtcggggctggcttaactatgcggcatca | |
| | gagcagattgtactgagagtgcaccataaaattgtaaacgttaatattttgttaaaattc | |
| | gcgttaaatttttgttaaatcagctcattttttaaccaataggccgaaatcggcaaaatc | |
| | ccttataaatcaaaagaatagcccgagatagggttgagtgttgttccagtttggaacaag | |
| | agtccactattaaagaacgtggactccaacgtcaaagggcgaaaaaccgtctatcagggc | |
| | gatggcccactacgtgaaccatcacccaaatcaagttttttggggtcgaggtgccgtaaa | |
| | gcactaaatcggaaccctaaagggagcccccgatttagagcttgacggggaaagccggcg | |
| | aacgtggcgagaaaggaagggaagaaagcgaaaggagcgggcgctagggcgctggca | |
| | agtgtagcggtcacgctgcgcgtaaccaccacacccgccgcgcttaatgcgccgctacag | |
| | ggcgcgtactatggttgctttgacgtatgcggtgtgaaataccgcacagatgcgtaaggaga | |
| | aaataccgcatcaggcgccattcgccattcaggctgcgcaactgttgggaagggcgatcgg | |
| | tgcgggcctcttcgctattacgccagctggcgaaagggggatgtgctgcaaggcgattaa | |
| | gttgggtaacgccagggttttcccagtcacgacgttgtaaaacgacggccagtgccaagc | |
| | tttaatacgactcactatagggaccaaacagagaatccgtgagttacgataaaaggcgaagg | |
| | agcaattgaagtcgcacgggtagaaggtgtgaatctcgagtgcgagcccgaagcacaaac | |
| | tcgagaaagccttctgccaacatgtcttccgtatttgatgagtacgaacagctcctcgcggctc | |
| | agactcgccccaatggagctcatggagggggagaaaaagggagtaccttaaaagtagacg | |
| | tcccggtattcactcttaacagtgatgacccagaagatagatggagctttgtggtattctgcctc | |
| | cggattgctgttagcgaagatgccaacaaaccactcaggcaaggtgctctcatatctcttttat | |
| | gctcccactcacaggtaatgaggaaccatgttgcccttgcagggaaacagaatgaagccac | |
| | attggccgtgcttgagattgatggctttgccaacggcacgccccagttcaacaataggagtg | |
| | gagtgtctgaagagagagcacagagatttgcgatgatagcaggatctctccctcgggcatg | |
| | cagcaacggaaccccgttcgtcacagccggggccgaagatgatgcaccagaagacatca | |
| | ccgataccctggagaggatcctctctatccaggctcaagtatgggtcacagtagcaaaagcc | |
| | atgactgcgtatgagactgcagatgagtcggaaacaaggcgaatcaataagtatatgcagc | |
| | aaggcagggtccaaaagaaatacatcctctaccccgtatgcaggagcacaatccaactcac | |
| | gatcagacagtctcttgcagtccgcatctttttggttagcgagctcaagagaggccgcaacac | |
| | ggcaggtggtacctctacttattataacctggtaggggacgtagactcatacatcaggaatac | |
| | cgggcttactgcattcttcttgacactcaagtacggaatcaacaccaagacatcagcccttgc | |
| | acttagtagcctctcaggcgacatccagaagatgaagcagctcatgcgtttgtatcggatgaa | |
| | aggagataatgcgccgtacatgacattacttggtgatagtgaccagatgagctttgcgcctgc | |
| | cgagtatgcacaactttactcctttgccatgggtatggcatcagtcctagataaaggtactggg | |
| | aaataccaatttgccagggactttatgagcacatcattctggagacttggagtagagtacgctc | |
| | aggctcagggaagtagcattaacgaggatatggctgccgagctaaagctaaccccagcag | |
| | caaggaggggcctggcagctgctgcccaacgggtctccgaggagaccagcagcatagac | |
| | atgcctactcaacaagtcggagtcctcactgggcttagcgagggggggtcccaagctctac | |
| | aaggcggatcgaatagatcgcaagggcaaccagaagccggggatggggagacccaattc | |
| | ctggatctgatgagagcggtagcaaatagcatgagggaggcgccaaactctgcacagggc | |
| | actccccaatcggggcctcccccaactcctgggccatcccaagataacgacaccgactgg | |
| | gggtattgatggacaaaacccagcctgcttccacaaaaacatcccaatgccctcacccgtag | |
| | tcgacccctcgatttgcggctctatatgaccacaccctcaaacaaacatccccctctttcctcc | |
| | ctccccctgctgtacaactacgtacgccctagataccacaggcacaatgcggctcactaaca | |
| | atcaaaacagagccgagggaattagaaaaaagtacgggtagaagagggatattcagagat | |
| | cagggcaagtctcccgagtctctgctctctcctctacctgatagaccaggacaaacatggcc | |
| | acctttacagatgcagagatcgacgagctatttgagacaagtggaactgtcattgacaacata | |
| | attacagcccagggtaaaccagcagagactgttggaaggagtgcaatcccacaaggcaag | |
| | accaaggtgctgagcgcagcatgggagaagcatgggagcatccagccaccggccagtca | |
| | agacaaccccgatcgacaggacagatctgacaaacaaccatccacacccgagcaaacga | |
| | ccccgcatgacagcccgccggccacatccgccgaccagccccccacccaggccacaga | |
| | cgaagccgtcgacacacagctcaggaccggagcaagcaactctctgctgttgatgcttgac | |
| | aagctcagcaataaatcgtccaatgctaaaaagggcccatggtcgagcccccaagagggg | |
| | aatcaccaacgtccgactcaacagcaggggagtcaacccagtcgcggaaacagtcagga | |
| | aagaccgcagaaccaagtcaaggccgcccctggaaaccagggcacagacgtgaacaca | |
| | gcatatcatggacaatgggaggagtcacaactatcagctggtgcaacccctcatgctctccg | |
| | atcaaggcagagccaagacaatacccttgtatctgcggatcatgtccagccacctgtagactt | |
| | tgtgcaagcgatgatgtctatgatggaggcgatatcacagagagtaagtaaggttgactatca | |
| | gctagatcttgtcttgaaacagacatcctccatccctatgatgcggtccgaaatccaacagctg | |
| | aaaacatctgttgcagtcatggaagccaacttgggaatgatgaagattctggatcccggttgt | |
| | gccaacatttcatctctgagtgatctacgggcagttgcccgatctcacccggttttagtttcagg | |
| | ccctggagacccctctccctatgtgacacaaggaggcgaaatggcacttaataaactttcgc | |
| | aaccagtgccacatccatctgaattgattaaacccgccactgcatgcgggcctgatatagga | |
| | gtggaaaaggacactgtccgtgcattgatcatgtcacgcccaatgcacccgagttcttcagc | |
| | caagctcctaagcaagttagatgcagccgggtcgatcgaggaaatcaggaaaatcaagcg | |
| | ccttgctctaaatggctaattactactgccacacgtagcgggtccctgtccactcggcatcaca | |
| | cggaatctgcaccgagttcccccCCGCGGTTAGAAAAAATACGGGT | |
| | AGAACCGCCACCATGGGTCACCAGCAGTTGGTCATCT | |
| | CTTGGTTTTCCCTGGTTTTTCTGGCATCTCCCCTCGT | |
| | GGCCATATGGGAACTGAAGAAAGATGTTTATGTCGTAG | |
| | AATTGGATTGGTATCCGGATGCCCCTGGAGAAATGGTG | |
| | GTCCTCACCTGTGACACCCCTGAAGAAGATGGTATCAC | |
| | CTGGACCTTGGACCAGAGCAGTGAGGTCTTAGGCTCTG | |
| | GCAAAACCCTGACCATCCAAGTCAAAGAGTTTGGAGAT | |
| | GCTGGCCAGTACACCTGTCACAAAGGAGGCGAGGTTCT | |
| | AAGCCATTCGCTCCTGCTGCTTCACAAAAAGGAAGATG | |
| | GAATTTGGTCCACTGATATTTTAAAGGACCAGAAAGAA | |
| | CCCAAAAATAAGACCTTTCTAAGATGCGAGGCCAAGAA | |
| | TTATTCTGGACGTTTCACCTGCTGGTGGCTGACGACAAT | |
| | CAGTACTGATTTGACATTCAGTGTCAAAAGCAGCAGAG | |
| | GCTCTTCTGACCCCCAAGGGGTGACGTGCGGAGCTGCT | |
| | ACACTCTCTGCAGAGAGAGTCAGAGGGGACAACAAGG | |
| | AGTATGAGTACTCAGTGGAGTGCCAGGAGGACAGTGCC | |
| | TGCCCAGCTGCTGAGGAGAGTCTGCCCATTGAGGTCAT | |
| | GGTGGATGCCGTTCACAAGCTCAAGTATGAAAACTACA | |
| | CCAGCAGCTTCTTCATCAGGGACATCATCAAACCTGAC | |
| | CCACCCAAGAACTTGCAGCTGAAGCCATTAAAGAATTC | |
| | TCGGCAGGTGGAGGTCAGCTGGGAGTACCCTGACACCT | |
| | GGAGTACTCCACATTCCTACTTCTCCCTGACATTCTGCG | |
| | TTCAGGTCCAGGGCAAGAGCAAGAGAGAAAAGAAAGA | |
| | TAGAGTCTTCACGGACAAGACCTCAGCCACGGTCATCT | |
| | GCCGCAAAAATGCCAGCATTAGCGTGCGGGCCCAGGAC | |
| | CGCTACTATAGCTCATCTTGGAGCGAATGGGCATCTGTG | |
| | CCCTGCAGTGGTGGCGGTGGCGGCGGATCTAGAAAC | |
| | CTCCCCGTGGCCACTCCAGACCCAGGAATGTTCCCATGC | |
| | CTTCACCACTCCCAAAACCTGCTGAGGGCCGTCAGCAA | |
| | CATGCTCCAGAAGGCCAGACAAACTCTAGAATTTTACC | |
| | CTTGCACTTCTGAAGAGATTGATCATGAAGATATCACA | |
| | AAAGATAAAACCAGCACAGTGGAGGCCTGTTTACCATT | |
| | GGAATTAACCAAGAATGAGAGTTGCCTAAATTCCAGAG | |
| | AGACCTCTTTCATAACTAATGGGAGTTGCCTGGCCTCCA | |
| | GAAAGACCTCTTTTATGATGGCCCTGTGCCTTAGTAGTA | |
| | TTTATGAAGACTCGAAGATGTACCAGGTGGAGTTCAAG | |
| | ACCATGAATGCAAAGCTTCTGATGGATCCTAAGAGGCA | |
| | GATCTTTCTAGATCAAAACATGCTGGCAGTTATTGATGA | |
| | GCTGATGCAGGCCCTGAATTTCAACAGTGAGACTGTGC | |
| | CACAAAAATCCTCCCTTGAAGAACCGGATTTTTATAAA | |
| | ACTAAAATCAAGCTCTGCATACTTCTTCATGCTTTCAGA | |
| | ATTCGGGCAGTGACTATTGATAGAGTGATGAGCTATCT | |
| | GAATGCTTCCTAATGATCCGCGGacccaaggtccaactctccaagcg | |
| | gcaatcctctctcgcttcctcagccccactgaatgatcgcgtaaccgtaattaatctagctacat | |
| | ttaagattaagaaaaaatacgggtagaattggagtgccccaattgtgccaagatggactcatc | |
| | taggacaattgggctgtactttgattctgcccattcttctagcaacctgttagcatttccgatcgt | |
| | cctacaagacacaggagatgggaagaagcaaatcgccccgcaatataggatccagcgcct | |
| | tgacttgtggactgatagtaaggaggactcagtattcatcaccacctatggattcatctttcaag | |
| | ttgggaatgaagaagccaccgtcggcatgatcgatgataaacccaagcgcgagttactttcc | |
| | gctgcgatgctctgcctaggaagcgtcccaaataccggagaccttattgagctggcaaggg | |
| | cctgtctcactatgatagtcacatgcaagaagagtgcaactaatactgagagaatggttttctc | |
| | agtagtgcaggcaccccaagtgctgcaaagctgtagggttgtggcaaacaaatactcatca | |
| | gtgaatgcagtcaagcacgtgaaagcgccagagaagattcccgggagtggaaccctagaa | |
| | tacaaggtgaactttgtctccttgactgtggtaccgaagagggatgtctacaagatcccagct | |
| | gcagtattgaaggtttctggctcgagtctgtacaatcttgcgctcaatgtcactattaatgtgga | |
| | ggtagacccgaggagtcctttggttaaatctctgtctaagtctgacagcggatactatgctaac | |
| | ctcttcttgcatattggacttatgaccactgtagataggaaggggaagaaagtgacatttgaca | |
| | agctggaaaagaaaataaggagccttgatctatctgtcgggctcagtgatgtgctcgggcctt | |
| | ccgtgttggtaaaagcaagaggtgcacggactaagcttttggcacctttcttctctagcagtgg | |
| | gacagcctgctatcccatagcaaatgcttctcctcaggtggccaagatactctggagtcaaac | |
| | cgcgtgcctgcggagcgttaaaatcattatccaagcaggtacccaacgcgctgtcgcagtg | |
| | accgccgaccacgaggttacctctactaagctggagaaggggcacacccttgccaaataca | |
| | atccttttaagaaataagctgcgtctctgagattgcgctccgcccactcacccagatcatcatg | |
| | acacaaaaaactaatctgtcttgattatttacagttagtttacctgtctatcaagttagaaaaaac | |
| | acgggtagaagattctggatcccggttggcgccctccaggtgcaagatgggctccagacct | |
| | tctaccaagaacccagcacctatgatgctgactatccgggttgcgctggtactgagttgcatct | |
| | gtccggcaaactccattgatggcaggcctcttgcagctgcaggaattgtggttacaggagac | |
| | aaagccgtcaacatatacacctcatcccagacaggatcaatcatagttaagctcctcccgaat | |
| | ctgcccaaggataaggaggcatgtgcgaaagcccccttggatgcatacaacaggacattga | |
| | ccactttgctcaccccccttggtgactctatccgtaggatacaagagtctgtgactacatctgg | |
| | aggggggagacaggggcgccttataggcgccattattggcggtgtggctcttggggttgca | |
| | actgccgcacaaataacagcggccgcagctctgatacaagccaaacaaaatgctgccaac | |
| | atcctccgacttaaagagagcattgccgcaaccaatgaggctgtgcatgaggtcactgacg | |
| | gattatcgcaactagcagtggcagttgggaagatgcagcagtttgttaatgaccaatttaataa | |
| | aacagctcaggaattagactgcatcaaaattgcacagcaagttggtgtagagctcaacctgta | |
| | cctaaccgaattgactacagtattcggaccacaaatcacttcacctgctttaaacaagctgact | |
| | attcaggcactttacaatctagctggtggaaatatggattacttattgactaagttaggtgtagg | |
| | gaacaatcaactcagctcattaatcggtagcggcttaatcaccggtaaccctattctatacgac | |
| | tcacagactcaactcttgggtatacaggtaactGCCccttcagtcgggaacctaaataatat | |
| | gcgtgccacctacttggaaaccttatccgtaagcacaaccaggggatttgcctcggcacttgt | |
| | cccaaaagtggtgacacaggtcggttctgtgatagaagaacttgacacctcatactgtataga | |
| | aactgacttagatttatattgtacaagaatagtaacgttccctatgtcccctggtatttattcctgct | |
| | tgagcggcaatacgtcggcctgtatgtactcaaagaccgaaggcgcacttactacaccatac | |
| | atgactatcaaaggttcagtcatcgccaactgcaagatgacaacatgtagatgtgtaaacccc | |
| | ccgggtatcatatcgcaaaactatggagaagccgtgtctctaatagataaacaatcatgcaat | |
| | gttttatccttaggcgggataactttaaggctcagtggggaattcgatgtaacttatcagaaga | |
| | atatctcaatacaagattctcaagtaataataacaggcaatcttgatatctcaactgagcttggg | |
| | aatgtcaacaactcgatcagtaatgctttgaataagttagaggaaagcaacagaaaactaga | |
| | caaagtcaatgtcaaactgactagcacatctgctctcattacctatatcgttttgactatcatatct | |
| | cttgtttttggtatacttagcctgattctagcatgctacctaatgtacaagcaaaaggcgcaaca | |
| | aaagaccttattatggcttgggaataatactctagatcagatgagagccactacaaaaatgtg | |
| | aacacagatgaggaacgaaggtttccctaatagtaatttgtgtgaaagttctggtagtctgtca | |
| | gttcagagagttaagaaaaaactaccggttgtagatgaccaaaggacgatatacgggtaga | |
| | acggtaagagaggccgcccctcaattgcgagccaggcttcacaacctccgttctaccgcttc | |
| | accgacaacagtcctcaatcatggaccgcgccgttagccaagttgcgttagagaatgatgaa | |
| | agagaggcaaaaaatacatggcgcttgatattccggattgcaatcttattcttaacagtagtga | |
| | ccttggctatatctgtagcctcccttttatatagcatgggggctagcacacctagcgatcttgta | |
| | ggcataccgactaggatttccagggcagaagaaaagattacatctacacttggttccaatcaa | |
| | gatgtagtagataggatatataagcaagtggcccttgagtctccgttggcattgttaaatactg | |
| | agaccacaattatgaacgcaataacatctctctcttatcagattaatggagctgcaaacaacag | |
| | tgggtggggggcacctatccatgacccagattatataggggggataggcaaagaactcatt | |
| | gtagatgatgctagtgatgtcacatcattctatccctctgcatttcaagaacatctgaattttatcc | |
| | cggcgcctactacaggatcaggttgcactcgaataccctcatttgacatgagtgctacccatt | |
| | actgctacacccataatgtaatattgtctggatgcagagatcactcacattcatatcagtatttag | |
| | cacttggtgtgctccggacatctgcaacagggagggtattcttttctactctgcgttccatcaac | |
| | ctggacgacacccaaaatcggaagtcttgcagtgtgagtgcaactcccctgggttgtgatat | |
| | gctgtgctcgaaagtcacggagacagaggaagaagattataactcagctgtccctacgcgg | |
| | atggtacatgggaggttagggttcgacggccagtaccacgaaaaggacctagatgtcacaa | |
| | cattattcggggactgggtggccaactacccaggagtagggggtggatcttttattgacagc | |
| | cgcgtatggttctcagtctacggagggttaaaacccaattcacccagtgacactgtacagga | |
| | agggaaatatgtgatatacaagcgatacaatgacacatgcccagatgagcaagactaccag | |
| | attcgaatggccaagtcttcgtataagcctggacggtttggtgggaaacgcatacagcaggc | |
| | tatcttatctatcaaggtgtcaacatccttaggcgaagacccggtactgactgtaccgcccaac | |
| | acagtcacactcatgggggccgaaggcagaattctcacagtagggacatctcatttcttgtat | |
| | caacgagggtcatcatacttctctcccgcgttattatatcctatgacagtcagcaacaaaacag | |
| | ccactcttcatagtccttatacattcaatgccttcactcggccaggtagtatcccttgccaggctt | |
| | cagcaagatgccccaactcgtgtgttactggagtctatacagatccatatcccctaatcttctat | |
| | agaaaccacaccttgcgaggggtattcgggacaatgcttgatggtgtacaagcaagacttaa | |
| | ccctgcgtctgcagtattcgatagcacatcccgcagtcgcattactcgagtgagttcaagcag | |
| | taccaaagcagcatacacaacatcaacttgttttaaagtggtcaagactaataagacctattgt | |
| | ctcagcattgctgaaatatctaatactctcttcggagaattcagaatcgtcccgttactagttga | |
| | gatcctcaaagatgacggggttagagaagccaggtctggctagttgagtcaattataaagga | |
| | gttggaaagatggcattgtatcacctatcttctgcgacatcaagaatcaaaccgaatgccggc | |
| | gcgtgctcgaattccatgttgccagttgaccacaatcagccagtgctcatgcgatcagattaa | |
| | gccttgtcaatagtctcttgattaagaaaaaatgtaagtggcaatgagatacaaggcaaaaca | |
| | gctcatggttaacaatacgggtaggacatggcgagctccggtcctgaaagggcagagcatc | |
| | agattatcctaccagagtcacacctgtcttcaccattggtcaagcacaaactactctattactgg | |
| | aaattaactgggctaccgcttcctgatgaatgtgacttcgaccacctcattctcagccgacaat | |
| | ggaaaaaaatacttgaatcggcctctcctgatactgagagaatgataaaactcggaagggca | |
| | gtacaccaaactcttaaccacaattccagaataaccggagtgctccaccccaggtgtttagaa | |
| | gaactggctaatattgaggtcccagattcaaccaacaaatttcggaagattgagaagaagatc | |
| | caaattcacaacacgagatatggagaactgttcacaaggctgtgtacgcatatagagaagaa | |
| | actgctggggtcatcttggtctaacaatgtcccccggtcagaggagttcagcagcattcgtac | |
| | ggatccggcattctggtttcactcaaaatggtccacagccaagtttgcatggctccatataaaa | |
| | cagatccagaggcatctgatggtggcagctaggacaaggtctgcggccaacaaattggtga | |
| | tgctaacccataaggtaggccaagtctttgtcactcctgaacttgtcgttgtgacgcatacgaa | |
| | tgagaacaagttcacatgtcttacccaggaacttgtattgatgtatgcagatatgatggagggc | |
| | agagatatggtcaacataatatcaaccacggcggtgcatctcagaagcttatcagagaaaatt | |
| | gatgacattttgcggttaatagacgctctggcaaaagacttgggtaatcaagtctacgatgttg | |
| | tatcactaatggagggatttgcatacggagctgtccagctactcgagccgtcaggtacatttg | |
| | caggagatttcttcgcattcaacctgcaggagcttaaagacattctaattggcctcctccccaa | |
| | tgatatagcagaatccgtgactcatgcaatcgctactgtattctctggtttagaacagaatcaag | |
| | cagctgagatgttgtgtctgttgcgtctgtggggtcacccactgcttgagtcccgtattgcagc | |
| | aaaggcagtcaggagccaaatgtgcgcaccgaaaatggtagactttgatatgatccttcagg | |
| | tactgtctttcttcaagggaacaatcatcaacgggtacagaaagaagaatgcaggtgtgtggc | |
| | cgcgagtcaaagtggatacaatatatgggaaggtcattgggcaactacatgcagattcagca | |
| | gagatttcacacgatatcatgttgagagagtataagagtttatctgcacttgaatttgagccatg | |
| | tatagaatatgaccctgtcaccaacctgagcatgttcctaaaagacaaggcaatcgcacacc | |
| | ccaacgataattggcttgcctcgtttaggcggaaccttctctccgaagaccagaagaaacatg | |
| | taaaagaagcaacttcgactaatcgcctcttgatagagtttttagagtcaaatgattttgatccat | |
| | ataaagagatggaatatctgacgacccttgagtaccttagagatgacaatgtggcagtatcat | |
| | actcgctcaaggagaaggaagtgaaagttaatggacggatcttcgctaagctgacaaagaa | |
| | gttaaggaactgtcaggtgatggcggaagggatcctagccgatcagattgcacctttctttca | |
| | gggaaatggagtcattcaggatagcatatccttgaccaagagtatgctagcgatgagtcaact | |
| | gtcttttaacagcaataagaaacgtatcactgactgtaaagaaagagtatcttcaaaccgcaat | |
| | catgatccgaaaagcaagaaccgtcggagagttgcaaccttcataacaactgacctgcaaa | |
| | agtactgtcttaattggagatatcagacaatcaaattgttcgctcatgccatcaatcagttgatg | |
| | ggcctacctcacttcttcgaatggattcacctaagactgatggacactacgatgttcgtaggag | |
| | accctttcaatcctccaagtgaccctactgactgtgacctctcaagagtccctaatgatgacat | |
| | atatattgtcagtgccagagggggtatcgaaggattatgccagaagctatggacaatgatctc | |
| | aattgctgcaatccaacttgctgcagctagatcgcattgtcgtgttgcctgtatggtacagggt | |
| | gataatcaagtaatagcagtaacgagagaggtaagatcagacgactctccggagatggtgtt | |
| | gacacagttgcatcaagccagtgataatttcttcaaggaattaattcatgtcaatcatttgattgg | |
| | ccataatttgaaggatcgtgaaaccatcaggtcagacacattcttcatatacagcaaacgaat | |
| | cttcaaagatggagcaatcctcagtcaagtcctcaaaaattcatctaaattagtgctagtgtca | |
| | ggtgatctcagtgaaaacaccgtaatgtcctgtgccaacattgcctctactgtagcacggctat | |
| | gcgagaacgggcttcccaaagacttctgttactatttaaactatataatgagttgtgtgcagac | |
| | atactttgactctgagttctccatcaccaacaattcgcaccccgatcttaatcagtcgtggattg | |
| | aggacatctcttttgtgcactcatatgttctgactcctgcccaattagggggactgagtaacctt | |
| | caatactcaaggctctacactagaaatatcggtgacccggggactactgcttttgcagagatc | |
| | aagcgactagaagcagtgggattactgagtcctaacattatgactaatatcttaactaggccg | |
| | cctgggaatggagattgggccagtctgtgcaacgacccatactctttcaattttgagactgttg | |
| | caagcccaaatattgttcttaagaaacatacgcaaagagtcctatttgaaacttgttcaaatccc | |
| | ttattgtctggagtgcacacagaggataatgaggcagaagagaaggcattggctgaattcttg | |
| | cttaatcaagaggtgattcatccccgcgttgcgcatgccatcatggaggcaagctctgtaggt | |
| | aggagaaagcaaattcaagggcttgttgacacaacaaacaccgtaattaagattgcgcttact | |
| | aggaggccattaggcatcaagaggctgatgcggatagtcaattattctagcatgcatgcaat | |
| | gctgtttagagacgatgttttttcctccagtagatccaaccaccccttagtctcttctaatatgtgt | |
| | tctctgacactggcagactatgcacggaatagaagctggtcacctttgacgggaggcagga | |
| | aaatactgggtgtatctaatcctgatacgatagaactcgtagagggtgagattcttagtgtaag | |
| | cggagggtgtacaagatgtgacagcggagatgaacaatttacttggttccatcttccaagcaa | |
| | tatagaattgaccgatgacaccagcaagaatcctccgatgagggtaccatatctcgggtcaa | |
| | agacacaggagaggagagctgcctcacttgcaaaaatagctcatatgtcgccacatgtaaa | |
| | ggctgccctaagggcatcatccgtgttgatctgggcttatggggataatgaagtaaattggac | |
| | tgctgctcttacgattgcaaaatctcggtgtaatgtaaacttagagtatcttcggttactgtcccc | |
| | tttacccacggctgggaatcttcaacatagactagatgatggtataactcagatgacattcacc | |
| | cctgcatctctctacagggtgtcaccttacattcacatatccaatgattctcaaaggctgttcact | |
| | gaagaaggagtcaaagaggggaatgtggtttaccaacagatcatgctcttgggtttatctcta | |
| | atcgaatcgatctttccaatgacaacaaccaggacatatgatgagatcacactgcacctacat | |
| | agtaaatttagttgctgtatcagagaagcacctgttgcggttcctttcgagctacttggggtggt | |
| | accggaactgaggacagtgacctcaaataagtttatgtatgatcctagccctgtatcggaggg | |
| | agactttgcgagacttgacttagctatcttcaagagttatgagcttaatctggagtcatatccca | |
| | cgatagagctaatgaacattctttcaatatccagcgggaagttgattggccagtctgtggtttct | |
| | tatgatgaagatacctccataaagaatgacgccataatagtgtatgacaatacccgaaattgg | |
| | atcagtgaagctcagaattcagatgtggtccgcctatttgaatatgcagcacttgaagtgctcc | |
| | tcgactgttcttaccaactctattacctgagagtaagaggcctggacaatattgtcttatatatgg | |
| | gtgatttatacaagaatatgccaggaattctactttccaacattgcagctacaatatctcatccc | |
| | gtcattcattcaaggttacatgcagtgggcctggtcaaccatgacggatcacaccaacttgca | |
| | gatacggattttatcgaaatgtctgcaaaactattagtatcttgcacccgacgtgtgatctccgg | |
| | cttatattcaggaaataagtatgatctgctgttcccatctgtcttagatgataacctgaatgagaa | |
| | gatgcttcagctgatatcccggttatgctgtctgtacacggtactctttgctacaacaagagaa | |
| | atcccgaaaataagaggcttaactgcagaagagaaatgttcaatactcactgagtatttactgt | |
| | cggatgctgtgaaaccattacttagccccgatcaagtgagctctatcatgtctcctaacataatt | |
| | acattcccagctaatctgtactacatgtctcggaagagcctcaatttgatcagggaaagggag | |
| | gacagggatactatcctggcgttgttgttcccccaagagccattattagagttcccttctgtgca | |
| | agatattggtgctcgagtgaaagatccattcacccgacaacctgcggcatttttgcaagagtta | |
| | gatttgagtgctccagcaaggtatgacgcattcacacttagtcagattcatcctgaactcacat | |
| | ctccaaatccggaggaagactacttagtacgatacttgttcagagggatagggactgcatctt | |
| | cctcttggtataaggcatctcatctcctttctgtacccgaggtaagatgtgcaagacacgggaa | |
| | ctccttatacttagctgaagggagcggagccatcatgagtcttctcgaactgcatgtaccacat | |
| | gaaactatctattacaatacgctcttttcaaatgagatgaaccccccgcaacgacatttcgggc | |
| | cgaccccaactcagtttttgaattcggttgtttataggaatctacaggcggaggtaacatgcaa | |
| | agatggatttgtccaagagttccgtccattatggagagaaaatacagaggaaagcgacctga | |
| | cctcagataaagtagtggggtatattacatctgcagtgccctacagatctgtatcattgctgcat | |
| | tgtgacattgaaattcctccagggtccaatcaaagcttactagatcaactagctatcaatttatct | |
| | ctgattgccatgcattctgtaagggagggcggggtagtaatcatcaaagtgttgtatgcaatg | |
| | ggatactactttcatctactcatgaacttgtttgctccgtgttccacaaaaggatatattctctcta | |
| | atggttatgcatgtcgaggagatatggagtgttacctggtatttgtcatgggttacctgggcgg | |
| | gcctacatttgtacatgaggtggtgaggatggcgaaaactctggtgcagcggcacggtacg | |
| | cttttgtctaaatcagatgagatcacactgaccaggttattcacctcacagcggcagcgtgtga | |
| | cagacatcctatccagtcctttaccaagattaataaagtacttgaggaagaatattgacactgc | |
| | gctgattgaagccgggggacagcccgtccgtccattctgtgcggagagtctggtgagcacg | |
| | ctagcgaacataactcagataacccagatcatcgctagtcacattgacacagttatccggtct | |
| | gtgatatatatggaagctgagggtgatctcgctgacacagtatttctatttaccccttacaatctc | |
| | tctactgacgggaaaaagaggacatcacttaaacagtgcacgagacagatcctagaggtta | |
| | caatactaggtcttagagtcgaaaatctcaataaaataggcgatataatcagcctagtgcttaa | |
| | aggcatgatctccatggaggaccttatcccactaaggacatacttgaagcatagtacctgccc | |
| | taaatatttgaaggctgtcctaggtattaccaaactcaaagaaatgtttacagacacttctgtact | |
| | gtacttgactcgtgctcaacaaaaattctacatgaaaactataggcaatgcagtcaaaggatat | |
| | tacagtaactgtgactcttaacgaaaatcacatattaataggctccttttttggccaattgtattctt | |
| | gttgatttaatcatattatgttagaaaaaagttgaaccctgactccttaggactcgaattcgaact | |
| | caaataaatgtcttaaaaaaaggttgcgcacaattattcttgagtgtagtctcgtcattcaccaa | |
| | atctttgtttggtgcgcgcggccggcatggtcccagcctcctcgctggcgccggctgggcaa | |
| | cattccgaggggaccgtcccctcggtaatggcgaatgggacgtcgactgctaacaaagccc | |
| | gaaaggaagctgagttggctgctgccaccgctgagcaataactagcataaccccttggggc | |
| | ctctaaacgggtcttgaggggttttttgctgaaaggaggaactatatgcgcgcagatctgtcat | |
| | gatgatcattgcaattggatccatatatagggcccgggttataattacctcaggtcgacgtccc | |
| | atggccattcgaattcgtaatcatggtcatagctgtttcctgtgtgaaattgttatccgctcacaa | |
| | ttccacacaacatacgagccggaagcataaagtgtaaagcctggggtgcctaatgagtgag | |
| | ctaactcacattaattgcgttgcgctcactgcccgctttccagtcgggaaacctgtcgtgccag | |
| | ctgcattaatgaatcggccaacgcgcggggagaggcggtttgcgtattgggcgc | |
| Exemplary | accaaacagagaatccgtgagttacgataaaaggcgaaggagcaattgaagtcgcacggg | 62 |
| nucleic acid | tagaaggtgtgaatctcgagtgcgagcccgaagcacaaactcgagaaagccttctgccaac | |
| sequence | atgtcttccgtatttgatgagtacgaacagctcctcgcggctcagactcgccccaatggagct | |
| encoding NDV | catggagggggagaaaaagggagtaccttaaaagtagacgtcccggtattcactcttaaca | |
| genome | gtgatgacccagaagatagatggagctttgtggtattctgcctccggattgctgttagcgaag | |
| encoding a | atgccaacaaaccactcaggcaaggtgctctcatatctcttttatgctcccactcacaggtaat | |
| human IL-12 | gaggaaccatgttgcccttgcagggaaacagaatgaagccacattggccgtgcttgagattg | |
| transgene | atggctttgccaacggcacgccccagttcaacaataggagtggagtgtctgaagagagagc | |
| (Version 2.1) | acagagatttgcgatgatagcaggatctctccctcgggcatgcagcaacggaaccccgttc | |
| | gtcacagccggggccgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaggaggggcctggca | |
| | gctgctgcccaacgggtctccgaggagaccagcagcatagacatgcctactcaacaagtc | |
| | ggagtcctcactgggcttagcgagggggggtcccaagctctacaaggcggatcgaataga | |
| | tcgcaagggcaaccagaagccggggatggggagacccaattcctggatctgatgagagc | |
| | ggtagcaaatagcatgagggaggcgccaaactctgcacagggcactccccaatcggggc | |
| | ctcccccaactcctgggccatcccaagataacgacaccgactgggggtattgatggacaaa | |
| | acccagcctgcttccacaaaaacatcccaatgccctcacccgtagtcgacccctcgatttgc | |
| | ggctctatatgaccacaccctcaaacaaacatccccctctttcctccctccccctgctgtacaa | |
| | ctacgtacgccctagataccacaggcacaatgcggctcactaacaatcaaaacagagccga | |
| | gggaattagaaaaaagtacgggtagaagagggatattcagagatcagggcaagtctcccg | |
| | agtctctgctctctcctctacctgatagaccaggacaaacatggccacctttacagatgcaga | |
| | gatcgacgagctatttgagacaagtggaactgtcattgacaacataattacagcccagggta | |
| | aaccagcagagactgttggaaggagtgcaatcccacaaggcaagaccaaggtgctgagc | |
| | gcagcatgggagaagcatgggagcatccagccaccggccagtcaagacaaccccgatcg | |
| | acaggacagatctgacaaacaaccatccacacccgagcaaacgaccccgcatgacagcc | |
| | cgccggccacatccgccgaccagccccccacccaggccacagacgaagccgtcgacac | |
| | acagctcaggaccggagcaagcaactctctgctgttgatgcttgacaagctcagcaataaat | |
| | cgtccaatgctaaaaagggcccatggtcgagcccccaagaggggaatcaccaacgtccga | |
| | ctcaacagcaggggagtcaacccagtcgcggaaacagtcaggaaagaccgcagaaccaa | |
| | gtcaaggccgcccctggaaaccagggcacagacgtgaacacagcatatcatggacaatgg | |
| | gaggagtcacaactatcagctggtgcaacccctcatgctctccgatcaaggcagagccaag | |
| | acaatacccttgtatctgcggatcatgtccagccacctgtagactttgtgcaagcgatgatgtc | |
| | tatgatggaggcgatatcacagagagtaagtaaggttgactatcagctagatcttgtcttgaaa | |
| | cagacatcctccatccctatgatgcggtccgaaatccaacagctgaaaacatctgttgcagtc | |
| | atggaagccaacttgggaatgatgaagattctggatcccggttgtgccaacatttcatctctga | |
| | gtgatctacgggcagttgcccgatctcacccggttttagtttcaggccctggagacccctctc | |
| | cctatgtgacacaaggaggcgaaatggcacttaataaactttcgcaaccagtgccacatcca | |
| | tctgaattgattaaacccgccactgcatgcgggcctgatataggagtggaaaaggacactgt | |
| | ccgtgcattgatcatgtcacgcccaatgcacccgagttcttcagccaagctcctaagcaagtt | |
| | agatgcagccgggtcgatcgaggaaatcaggaaaatcaagcgccttgctctaaatggctaa | |
| | ttactactgccacacgtagcgggtccctgtccactcggcatcacacggaatctgcaccgagtt | |
| | cccccCCGCGGTTAGAAAAAATACGGGTAGAACCGCCAC | |
| | CATGTGCCATCAGCAGCTGGTCATCTCATGGTTCTCCCT | |
| | GGTGTTTCTGGCCTCACCTCTGGTCGCAATCTGGGAACT | |
| | GAAAAAGGATGTGTACGTGGTGGAGCTGGACTGGTATC | |
| | CCGATGCCCCTGGCGAGATGGTGGTGCTGACCTGCGAC | |
| | ACACCCGAGGAGGATGGCATCACCTGGACACTGGATCA | |
| | GAGCTCCGAGGTGCTGGGAAGCGGCAAGACCCTGACAA | |
| | TCCAGGTGAAGGAGTTCGGCGACGCCGGCCAGTACACC | |
| | TGTCACAAGGGAGGAGAGGTGCTGAGCCACTCCCTGCT | |
| | GCTGCTGCACAAGAAGGAGGATGGCATCTGGTCCACAG | |
| | ACATCCTGAAGGATCAGAAGGAGCCAAAGAACAAGAC | |
| | CTTCCTGCGGTGCGAGGCCAAGAATTATAGCGGCCGGT | |
| | TCACCTGTTGGTGGCTGACCACAATCTCCACCGATCTGA | |
| | CATTTTCTGTGAAGTCTAGCAGGGGATCCTCTGACCCAC | |
| | AGGGAGTGACATGCGGAGCAGCCACCCTGAGCGCCGA | |
| | GAGGGTGCGCGGCGATAACAAGGAGTACGAGTATTCCG | |
| | TGGAGTGCCAGGAGGACTCTGCCTGTCCAGCAGCAGAG | |
| | GAGTCCCTGCCTATCGAAGTGATGGTGGATGCCGTGCA | |
| | CAAGCTGAAGTACGAGAATTATACCAGCTCCTTCTTTAT | |
| | CCGGGACATCATCAAGCCCGATCCCCCTAAGAACCTGC | |
| | AGCTGAAGCCTCTGAAGAATAGCAGACAGGTGGAGGTG | |
| | TCCTGGGAGTACCCTGACACCTGGAGCACACCACACTC | |
| | CTATTTCTCTCTGACCTTTTGCGTGCAGGTGCAGGGCAA | |
| | GTCCAAGCGGGAGAAGAAGGACAGAGTGTTCACCGAT | |
| | AAGACATCTGCCACCGTGATCTGTAGAAAGAACGCCTC | |
| | TATCAGCGTGAGGGCCCAGGACCGCTACTATTCTAGCT | |
| | CCTGGTCCGAGTGGGCCTCTGTGCCTTGCAGCGGCGGA | |
| | GGAGGAGGAGGATCTAGGAATCTGCCAGTGGCAACCCC | |
| | TGACCCAGGCATGTTCCCCTGCCTGCACCACAGCCAGA | |
| | ACCTGCTGAGGGCCGTGTCCAATATGCTGCAGAAGGCC | |
| | CGCCAGACACTGGAGTTTTACCCTTGTACCAGCGAGGA | |
| | GATCGACCACGAGGACATCACAAAGGATAAGACCTCCA | |
| | CAGTGGAGGCCTGCCTGCCACTGGAGCTGACCAAGAAC | |
| | GAGTCCTGTCTGAACAGCCGGGAGACAAGCTTCATCAC | |
| | CAACGGCTCCTGCCTGGCCTCTAGAAAGACAAGCTTTA | |
| | TGATGGCCCTGTGCCTGTCTAGCATCTACGAGGACCTGA | |
| | AGATGTATCAGGTGGAGTTCAAGACCATGAACGCCAAG | |
| | CTGCTGATGGACCCCAAGAGGCAGATCTTTCTGGATCA | |
| | GAATATGCTGGCCGTGATCGACGAGCTGATGCAGGCCC | |
| | TGAACTTCAATAGCGAGACAGTGCCTCAGAAGTCCTCT | |
| | CTGGAGGAGCCAGATTTCTACAAGACCAAGATCAAGCT | |
| | GTGCATCCTGCTGCACGCCTTTCGGATCAGAGCCGTGAC | |
| | AATCGACCGCGTGATGTCCTATCTGAATGCTTCCTAATG | |
| | ATCCGCGGacccaaggtccaactctccaagcggcaatcctctctcgcttcctcagccc | |
| | cactgaatgatcgcgtaaccgtaattaatctagctacatttaagattaagaaaaaatacgggta | |
| | gaattggagtgccccaattgtgccaagatggactcatctaggacaattgggctgtactttgatt | |
| | ctgcccattcttctagcaacctgttagcatttccgatcgtcctacaagacacaggagatggga | |
| | agaagcaaatcgccccgcaatataggatccagcgccttgacttgtggactgatagtaagga | |
| | ggactcagtattcatcaccacctatggattcatctttcaagttgggaatgaagaagccaccgtc | |
| | ggcatgatcgatgataaacccaagcgcgagttactttccgctgcgatgctctgcctaggaag | |
| | cgtcccaaataccggagaccttattgagctggcaagggcctgtctcactatgatagtcacatg | |
| | caagaagagtgcaactaatactgagagaatggttttctcagtagtgcaggcaccccaagtgc | |
| | tgcaaagctgtagggttgtggcaaacaaatactcatcagtgaatgcagtcaagcacgtgaaa | |
| | gcgccagagaagattcccgggagtggaaccctagaatacaaggtgaactttgtctccttgac | |
| | tgtggtaccgaagagggatgtctacaagatcccagctgcagtattgaaggtttctggctcgag | |
| | tctgtacaatcttgcgctcaatgtcactattaatgtggaggtagacccgaggagtcctttggtta | |
| | aatctctgtctaagtctgacagcggatactatgctaacctcttcttgcatattggacttatgacca | |
| | ctgtagataggaaggggaagaaagtgacatttgacaagctggaaaagaaaataaggagcc | |
| | ttgatctatctgtcgggctcagtgatgtgctcgggccttccgtgttggtaaaagcaagaggtgc | |
| | acggactaagcttttggcacctttcttctctagcagtgggacagcctgctatcccatagcaaat | |
| | gcttctcctcaggtggccaagatactctggagtcaaaccgcgtgcctgcggagcgttaaaat | |
| | cattatccaagcaggtacccaacgcgctgtcgcagtgaccgccgaccacgaggttacctct | |
| | actaagctggagaaggggcacacccttgccaaatacaatccttttaagaaataagctgcgtct | |
| | ctgagattgcgctccgcccactcacccagatcatcatgacacaaaaaactaatctgtcttgatt | |
| | atttacagttagtttacctgtctatcaagttagaaaaaacacgggtagaagattctggatcccg | |
| | gttggcgccctccaggtgcaagatgggctccagaccttctaccaagaacccagcacctatg | |
| | atgctgactatccgggttgcgctggtactgagttgcatctgtccggcaaactccattgatggca | |
| | ggcctcttgcagctgcaggaattgtggttacaggagacaaagccgtcaacatatacacctca | |
| | tcccagacaggatcaatcatagttaagctcctcccgaatctgcccaaggataaggaggcatg | |
| | tgcgaaagcccccttggatgcatacaacaggacattgaccactttgctcaccccccttggtga | |
| | ctctatccgtaggatacaagagtctgtgactacatctggaggggggagacaggggcgcctt | |
| | ataggcgccattattggcggtgtggctcttggggttgcaactgccgcacaaataacagcggc | |
| | cgcagctctgatacaagccaaacaaaatgctgccaacatcctccgacttaaagagagcattg | |
| | ccgcaaccaatgaggctgtgcatgaggtcactgacggattatcgcaactagcagtggcagtt | |
| | gggaagatgcagcagtttgttaatgaccaatttaataaaacagctcaggaattagactgcatc | |
| | aaaattgcacagcaagttggtgtagagctcaacctgtacctaaccgaattgactacagtattc | |
| | ggaccacaaatcacttcacctgctttaaacaagctgactattcaggcactttacaatctagctg | |
| | gtggaaatatggattacttattgactaagttaggtgtagggaacaatcaactcagctcattaatc | |
| | ggtagcggcttaatcaccggtaaccctattctatacgactcacagactcaactcttgggtatac | |
| | aggtaactGCCccttcagtcgggaacctaaataatatgcgtgccacctacttggaaacctta | |
| | tccgtaagcacaaccaggggatttgcctcggcacttgtcccaaaagtggtgacacaggtcg | |
| | gttctgtgatagaagaacttgacacctcatactgtatagaaactgacttagatttatattgtacaa | |
| | gaatagtaacgttccctatgtcccctggtatttattcctgcttgagcggcaatacgtcggcctgt | |
| | atgtactcaaagaccgaaggcgcacttactacaccatacatgactatcaaaggttcagtcatc | |
| | gccaactgcaagatgacaacatgtagatgtgtaaaccccccgggtatcatatcgcaaaacta | |
| | tggagaagccgtgtctctaatagataaacaatcatgcaatgttttatccttaggcgggataactt | |
| | taaggctcagtggggaattcgatgtaacttatcagaagaatatctcaatacaagattctcaagt | |
| | aataataacaggcaatcttgatatctcaactgagcttgggaatgtcaacaactcgatcagtaat | |
| | gctttgaataagttagaggaaagcaacagaaaactagacaaagtcaatgtcaaactgactag | |
| | cacatctgctctcattacctatatcgttttgactatcatatctcttgtttttggtatacttagcctgat | |
| | tctagcatgctacctaatgtacaagcaaaaggcgcaacaaaagaccttattatggcttgggaat | |
| | aatactctagatcagatgagagccactacaaaaatgtgaacacagatgaggaacgaaggttt | |
| | ccctaatagtaatttgtgtgaaagttctggtagtctgtcagttcagagagttaagaaaaaactac | |
| | cggttgtagatgaccaaaggacgatatacgggtagaacggtaagagaggccgcccctcaa | |
| | ttgcgagccaggcttcacaacctccgttctaccgcttcaccgacaacagtcctcaatcatgga | |
| | ccgcgccgttagccaagttgcgttagagaatgatgaaagagaggcaaaaaatacatggcgc | |
| | ttgatattccggattgcaatcttattcttaacagtagtgaccttggctatatctgtagcctccctttt | |
| | atatagcatgggggctagcacacctagcgatcttgtaggcataccgactaggatttccaggg | |
| | cagaagaaaagattacatctacacttggttccaatcaagatgtagtagataggatatataagca | |
| | agtggcccttgagtctccgttggcattgttaaatactgagaccacaattatgaacgcaataaca | |
| | tctctctcttatcagattaatggagctgcaaacaacagtgggtggggggcacctatccatgac | |
| | ccagattatataggggggataggcaaagaactcattgtagatgatgctagtgatgtcacatca | |
| | ttctatccctctgcatttcaagaacatctgaattttatcccggcgcctactacaggatcaggttgc | |
| | actcgaataccctcatttgacatgagtgctacccattactgctacacccataatgtaatattgtct | |
| | ggatgcagagatcactcacattcatatcagtatttagcacttggtgtgctccggacatctgcaa | |
| | cagggagggtattcttttctactctgcgttccatcaacctggacgacacccaaaatcggaagt | |
| | cttgcagtgtgagtgcaactcccctgggttgtgatatgctgtgctcgaaagtcacggagacag | |
| | aggaagaagattataactcagctgtccctacgcggatggtacatgggaggttagggttcgac | |
| | ggccagtaccacgaaaaggacctagatgtcacaacattattcggggactgggtggccaact | |
| | acccaggagtagggggtggatcttttattgacagccgcgtatggttctcagtctacggagggt | |
| | taaaacccaattcacccagtgacactgtacaggaagggaaatatgtgatatacaagcgatac | |
| | aatgacacatgcccagatgagcaagactaccagattcgaatggccaagtcttcgtataagcc | |
| | tggacggtttggtgggaaacgcatacagcaggctatcttatctatcaaggtgtcaacatcctta | |
| | ggcgaagacccggtactgactgtaccgcccaacacagtcacactcatgggggccgaagg | |
| | cagaattctcacagtagggacatctcatttcttgtatcaacgagggtcatcatacttctctcccg | |
| | cgttattatatcctatgacagtcagcaacaaaacagccactcttcatagtccttatacattcaatg | |
| | ccttcactcggccaggtagtatcccttgccaggcttcagcaagatgccccaactcgtgtgtta | |
| | ctggagtctatacagatccatatcccctaatcttctatagaaaccacaccttgcgaggggtatt | |
| | cgggacaatgcttgatggtgtacaagcaagacttaaccctgcgtctgcagtattcgatagcac | |
| | atcccgcagtcgcattactcgagtgagttcaagcagtaccaaagcagcatacacaacatcaa | |
| | cttgttttaaagtggtcaagactaataagacctattgtctcagcattgctgaaatatctaatactct | |
| | cttcggagaattcagaatcgtcccgttactagttgagatcctcaaagatgacggggttagaga | |
| | agccaggtctggctagttgagtcaattataaaggagttggaaagatggcattgtatcacctatc | |
| | ttctgcgacatcaagaatcaaaccgaatgccggcgcgtgctcgaattccatgttgccagttga | |
| | ccacaatcagccagtgctcatgcgatcagattaagccttgtcaatagtctcttgattaagaaaa | |
| | aatgtaagtggcaatgagatacaaggcaaaacagctcatggttaacaatacgggtaggacat | |
| | ggcgagctccggtcctgaaagggcagagcatcagattatcctaccagagtcacacctgtctt | |
| | caccattggtcaagcacaaactactctattactggaaattaactgggctaccgcttcctgatga | |
| | atgtgacttcgaccacctcattctcagccgacaatggaaaaaaatacttgaatcggcctctcct | |
| | gatactgagagaatgataaaactcggaagggcagtacaccaaactcttaaccacaattccag | |
| | aataaccggagtgctccaccccaggtgtttagaagaactggctaatattgaggtcccagattc | |
| | aaccaacaaatttcggaagattgagaagaagatccaaattcacaacacgagatatggagaa | |
| | ctgttcacaaggctgtgtacgcatatagagaagaaactgctggggtcatcttggtctaacaat | |
| | gtcccccggtcagaggagttcagcagcattcgtacggatccggcattctggtttcactcaaaa | |
| | tggtccacagccaagtttgcatggctccatataaaacagatccagaggcatctgatggtggc | |
| | agctaggacaaggtctgcggccaacaaattggtgatgctaacccataaggtaggccaagtc | |
| | tttgtcactcctgaacttgtcgttgtgacgcatacgaatgagaacaagttcacatgtcttaccca | |
| | ggaacttgtattgatgtatgcagatatgatggagggcagagatatggtcaacataatatcaac | |
| | cacggcggtgcatctcagaagcttatcagagaaaattgatgacattttgcggttaatagacgc | |
| | tctggcaaaagacttgggtaatcaagtctacgatgttgtatcactaatggagggatttgcatac | |
| | ggagctgtccagctactcgagccgtcaggtacatttgcaggagatttcttcgcattcaacctg | |
| | caggagcttaaagacattctaattggcctcctccccaatgatatagcagaatccgtgactcatg | |
| | caatcgctactgtattctctggtttagaacagaatcaagcagctgagatgttgtgtctgttgcgt | |
| | ctgtggggtcacccactgcttgagtcccgtattgcagcaaaggcagtcaggagccaaatgtg | |
| | cgcaccgaaaatggtagactttgatatgatccttcaggtactgtctttcttcaagggaacaatca | |
| | tcaacgggtacagaaagaagaatgcaggtgtgtggccgcgagtcaaagtggatacaatata | |
| | tgggaaggtcattgggcaactacatgcagattcagcagagatttcacacgatatcatgttgag | |
| | agagtataagagtttatctgcacttgaatttgagccatgtatagaatatgaccctgtcaccaacc | |
| | tgagcatgttcctaaaagacaaggcaatcgcacaccccaacgataattggcttgcctcgttta | |
| | ggcggaaccttctctccgaagaccagaagaaacatgtaaaagaagcaacttcgactaatcg | |
| | cctcttgatagagtttttagagtcaaatgattttgatccatataaagagatggaatatctgacgac | |
| | ccttgagtaccttagagatgacaatgtggcagtatcatactcgctcaaggagaaggaagtga | |
| | aagttaatggacggatcttcgctaagctgacaaagaagttaaggaactgtcaggtgatggcg | |
| | gaagggatcctagccgatcagattgcacctttctttcagggaaatggagtcattcaggatagc | |
| | atatccttgaccaagagtatgctagcgatgagtcaactgtcttttaacagcaataagaaacgta | |
| | tcactgactgtaaagaaagagtatcttcaaaccgcaatcatgatccgaaaagcaagaaccgt | |
| | cggagagttgcaaccttcataacaactgacctgcaaaagtactgtcttaattggagatatcag | |
| | acaatcaaattgttcgctcatgccatcaatcagttgatgggcctacctcacttcttcgaatggatt | |
| | cacctaagactgatggacactacgatgttcgtaggagaccctttcaatcctccaagtgaccct | |
| | actgactgtgacctctcaagagtccctaatgatgacatatatattgtcagtgccagagggggt | |
| | atcgaaggattatgccagaagctatggacaatgatctcaattgctgcaatccaacttgctgca | |
| | gctagatcgcattgtcgtgttgcctgtatggtacagggtgataatcaagtaatagcagtaacga | |
| | gagaggtaagatcagacgactctccggagatggtgttgacacagttgcatcaagccagtgat | |
| | aatttcttcaaggaattaattcatgtcaatcatttgattggccataatttgaaggatcgtgaaacc | |
| | atcaggtcagacacattcttcatatacagcaaacgaatcttcaaagatggagcaatcctcagt | |
| | caagtcctcaaaaattcatctaaattagtgctagtgtcaggtgatctcagtgaaaacaccgtaa | |
| | tgtcctgtgccaacattgcctctactgtagcacggctatgcgagaacgggcttcccaaagact | |
| | tctgttactatttaaactatataatgagttgtgtgcagacatactttgactctgagttctccatcacc | |
| | aacaattcgcaccccgatcttaatcagtcgtggattgaggacatctcttttgtgcactcatatgtt | |
| | ctgactcctgcccaattagggggactgagtaaccttcaatactcaaggctctacactagaaat | |
| | atcggtgacccggggactactgcttttgcagagatcaagcgactagaagcagtgggattact | |
| | gagtcctaacattatgactaatatcttaactaggccgcctgggaatggagattgggccagtct | |
| | gtgcaacgacccatactctttcaattttgagactgttgcaagcccaaatattgttcttaagaaac | |
| | atacgcaaagagtcctatttgaaacttgttcaaatcccttattgtctggagtgcacacagaggat | |
| | aatgaggcagaagagaaggcattggctgaattcttgcttaatcaagaggtgattcatccccgc | |
| | gttgcgcatgccatcatggaggcaagctctgtaggtaggagaaagcaaattcaagggcttgt | |
| | tgacacaacaaacaccgtaattaagattgcgcttactaggaggccattaggcatcaagaggc | |
| | tgatgcggatagtcaattattctagcatgcatgcaatgctgtttagagacgatgttttttcctcca | |
| | gtagatccaaccaccccttagtctcttctaatatgtgttctctgacactggcagactatgcacgg | |
| | aatagaagctggtcacctttgacgggaggcaggaaaatactgggtgtatctaatcctgatac | |
| | gatagaactcgtagagggtgagattcttagtgtaagcggagggtgtacaagatgtgacagc | |
| | ggagatgaacaatttacttggttccatcttccaagcaatatagaattgaccgatgacaccagca | |
| | agaatcctccgatgagggtaccatatctcgggtcaaagacacaggagaggagagctgcctc | |
| | acttgcaaaaatagctcatatgtcgccacatgtaaaggctgccctaagggcatcatccgtgtt | |
| | gatctgggcttatggggataatgaagtaaattggactgctgctcttacgattgcaaaatctcgg | |
| | tgtaatgtaaacttagagtatcttcggttactgtcccctttacccacggctgggaatcttcaacat | |
| | agactagatgatggtataactcagatgacattcacccctgcatctctctacagggtgtcacctt | |
| | acattcacatatccaatgattctcaaaggctgttcactgaagaaggagtcaaagaggggaat | |
| | gtggtttaccaacagatcatgctcttgggtttatctctaatcgaatcgatctttccaatgacaaca | |
| | accaggacatatgatgagatcacactgcacctacatagtaaatttagttgctgtatcagagaa | |
| | gcacctgttgcggttcctttcgagctacttggggtggtaccggaactgaggacagtgacctca | |
| | aataagtttatgtatgatcctagccctgtatcggagggagactttgcgagacttgacttagctat | |
| | cttcaagagttatgagcttaatctggagtcatatcccacgatagagctaatgaacattctttcaat | |
| | atccagcgggaagttgattggccagtctgtggtttcttatgatgaagatacctccataaagaat | |
| | gacgccataatagtgtatgacaatacccgaaattggatcagtgaagctcagaattcagatgtg | |
| | gtccgcctatttgaatatgcagcacttgaagtgctcctcgactgttcttaccaactctattacctg | |
| | agagtaagaggcctggacaatattgtcttatatatgggtgatttatacaagaatatgccaggaa | |
| | ttctactttccaacattgcagctacaatatctcatcccgtcattcattcaaggttacatgcagtgg | |
| | gcctggtcaaccatgacggatcacaccaacttgcagatacggattttatcgaaatgtctgcaa | |
| | aactattagtatcttgcacccgacgtgtgatctccggcttatattcaggaaataagtatgatctg | |
| | ctgttcccatctgtcttagatgataacctgaatgagaagatgcttcagctgatatcccggttatg | |
| | ctgtctgtacacggtactctttgctacaacaagagaaatcccgaaaataagaggcttaactgc | |
| | agaagagaaatgttcaatactcactgagtatttactgtcggatgctgtgaaaccattacttagcc | |
| | ccgatcaagtgagctctatcatgtctcctaacataattacattcccagctaatctgtactacatgt | |
| | ctcggaagagcctcaatttgatcagggaaagggaggacagggatactatcctggcgttgttg | |
| | ttcccccaagagccattattagagttcccttctgtgcaagatattggtgctcgagtgaaagatc | |
| | cattcacccgacaacctgcggcatttttgcaagagttagatttgagtgctccagcaaggtatga | |
| | cgcattcacacttagtcagattcatcctgaactcacatctccaaatccggaggaagactactta | |
| | gtacgatacttgttcagagggatagggactgcatcttcctcttggtataaggcatctcatctcct | |
| | ttctgtacccgaggtaagatgtgcaagacacgggaactccttatacttagctgaagggagcg | |
| | gagccatcatgagtcttctcgaactgcatgtaccacatgaaactatctattacaatacgctctttt | |
| | caaatgagatgaaccccccgcaacgacatttcgggccgaccccaactcagtttttgaattcg | |
| | gttgtttataggaatctacaggcggaggtaacatgcaaagatggatttgtccaagagttccgtc | |
| | cattatggagagaaaatacagaggaaagcgacctgacctcagataaagtagtggggtatatt | |
| | acatctgcagtgccctacagatctgtatcattgctgcattgtgacattgaaattcctccagggtc | |
| | caatcaaagcttactagatcaactagctatcaatttatctctgattgccatgcattctgtaaggga | |
| | gggcggggtagtaatcatcaaagtgttgtatgcaatgggatactactttcatctactcatgaact | |
| | tgtttgctccgtgttccacaaaaggatatattctctctaatggttatgcatgtcgaggagatatgg | |
| | agtgttacctggtatttgtcatgggttacctgggcgggcctacatttgtacatgaggtggtgag | |
| | gatggcgaaaactctggtgcagcggcacggtacgcttttgtctaaatcagatgagatcacact | |
| | gaccaggttattcacctcacagcggcagcgtgtgacagacatcctatccagtcctttaccaag | |
| | attaataaagtacttgaggaagaatattgacactgcgctgattgaagccgggggacagcccg | |
| | tccgtccattctgtgcggagagtctggtgagcacgctagcgaacataactcagataacccag | |
| | atcatcgctagtcacattgacacagttatccggtctgtgatatatatggaagctgagggtgatct | |
| | cgctgacacagtatttctatttaccccttacaatctctctactgacgggaaaaagaggacatca | |
| | cttaaacagtgcacgagacagatcctagaggttacaatactaggtcttagagtcgaaaatctc | |
| | aataaaataggcgatataatcagcctagtgcttaaaggcatgatctccatggaggaccttatc | |
| | ccactaaggacatacttgaagcatagtacctgccctaaatatttgaaggctgtcctaggtatta | |
| | ccaaactcaaagaaatgtttacagacacttctgtactgtacttgactcgtgctcaacaaaaattc | |
| | tacatgaaaactataggcaatgcagtcaaaggatattacagtaactgtgactcttaacgaaaat | |
| | cacatattaataggctccttttttggccaattgtattcttgttgatttaatcatattatgttagaaaaa | |
| | agttgaaccctgactccttaggactcgaattcgaactcaaataaatgtcttaaaaaaaggttgc | |
| | gcacaattattcttgagtgtagtctcgtcattcaccaaatctttgtttggt | |
| Exemplary | accaaacagagaatccgtgagttacgataaaaggcgaaggagcaattgaagtcgcacggg | 67 |
| nucleic acid | tagaaggtgtgaatctcgagtgcgagcccgaagcacaaactcgagaaagccttctgccaac | |
| sequence | atgtcttccgtatttgatgagtacgaacagctcctcgcggctcagactcgccccaatggagct | |
| encoding NDV | catggagggggagaaaaagggagtaccttaaaagtagacgtcccggtattcactcttaaca | |
| genome | gtgatgacccagaagatagatggagctttgtggtattctgcctccggattgctgttagcgaag | |
| encoding a | atgccaacaaaccactcaggcaaggtgctctcatatctcttttatgctcccactcacaggtaat | |
| human IL-12 | gaggaaccatgttgcccttgcagggaaacagaatgaagccacattggccgtgcttgagattg | |
| transgene | atggctttgccaacggcacgccccagttcaacaataggagtggagtgtctgaagagagagc | |
| (Version 3) | acagagatttgcgatgatagcaggatctctccctcgggcatgcagcaacggaaccccgttc | |
| | gtcacagccggggccgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaggaggggcctggca | |
| | gctgctgcccaacgggtctccgaggagaccagcagcatagacatgcctactcaacaagtc | |
| | ggagtcctcactgggcttagcgagggggggtcccaagctctacaaggcggatcgaataga | |
| | tcgcaagggcaaccagaagccggggatggggagacccaattcctggatctgatgagagc | |
| | ggtagcaaatagcatgagggaggcgccaaactctgcacagggcactccccaatcggggc | |
| | ctcccccaactcctgggccatcccaagataacgacaccgactgggggtattgatggacaaa | |
| | acccagcctgcttccacaaaaacatcccaatgccctcacccgtagtcgacccctcgatttgc | |
| | ggctctatatgaccacaccctcaaacaaacatccccctctttcctccctccccctgctgtacaa | |
| | ctacgtacgccctagataccacaggcacaatgcggctcactaacaatcaaaacagagccga | |
| | gggaattagaaaaaagtacgggtagaagagggatattcagagatcagggcaagtctcccg | |
| | agtctctgctctctcctctacctgatagaccaggacaaacatggccacctttacagatgcaga | |
| | gatcgacgagctatttgagacaagtggaactgtcattgacaacataattacagcccagggta | |
| | aaccagcagagactgttggaaggagtgcaatcccacaaggcaagaccaaggtgctgagc | |
| | gcagcatgggagaagcatgggagcatccagccaccggccagtcaagacaaccccgatcg | |
| | acaggacagatctgacaaacaaccatccacacccgagcaaacgaccccgcatgacagcc | |
| | cgccggccacatccgccgaccagccccccacccaggccacagacgaagccgtcgacac | |
| | acagctcaggaccggagcaagcaactctctgctgttgatgcttgacaagctcagcaataaat | |
| | cgtccaatgctaaaaagggcccatggtcgagcccccaagaggggaatcaccaacgtccga | |
| | ctcaacagcaggggagtcaacccagtcgcggaaacagtcaggaaagaccgcagaaccaa | |
| | gtcaaggccgcccctggaaaccagggcacagacgtgaacacagcatatcatggacaatgg | |
| | gaggagtcacaactatcagctggtgcaacccctcatgctctccgatcaaggcagagccaag | |
| | acaatacccttgtatctgcggatcatgtccagccacctgtagactttgtgcaagcgatgatgtc | |
| | tatgatggaggcgatatcacagagagtaagtaaggttgactatcagctagatcttgtcttgaaa | |
| | cagacatcctccatccctatgatgcggtccgaaatccaacagctgaaaacatctgttgcagtc | |
| | atggaagccaacttgggaatgatgaagattctggatcccggttgtgccaacatttcatctctga | |
| | gtgatctacgggcagttgcccgatctcacccggttttagtttcaggccctggagacccctctc | |
| | cctatgtgacacaaggaggcgaaatggcacttaataaactttcgcaaccagtgccacatcca | |
| | tctgaattgattaaacccgccactgcatgcgggcctgatataggagtggaaaaggacactgt | |
| | ccgtgcattgatcatgtcacgcccaatgcacccgagttcttcagccaagctcctaagcaagtt | |
| | agatgcagccgggtcgatcgaggaaatcaggaaaatcaagcgccttgctctaaatggctaa | |
| | ttactactgccacacgtagcgggtccctgtccactcggcatcacacggaatctgcaccgagtt | |
| | cccccCCGCGGTTAGAAAAAATACGGGTAGAACCGCCAC | |
| | Catgtgtcaccagcagttggtcatctcttggttttccctggtttttctggcatctcccctcgtggc | |
| | catatgggaactgaagaaagatgtttatgtcgtagaattggattggtatccggatgcccctgg | |
| | agaaatggtggtcctcacctgtgacacccctgaagaagatggtatcacctggaccttggacc | |
| | agagcagtgaggtcttaggctctggcaaaaccctgaccatccaagtcaaagagtttggagat | |
| | gctggccagtacacctgtcacaaaggaggcgaggttctaagccattcgctcctgctgcttca | |
| | caaaaaggaagatggaatttggtccactgatattttaaaggaccagaaagaacccaaaaata | |
| | agacctttctaagatgcgaggccaagaattattctggacgtttcacctgctggtggctgacga | |
| | caatcagtactgatttgacattcagtgtcaaaagcagcagaggctcttctgacccccaaggg | |
| | gtgacgtgcggagctgctacactctctgcagagagagtcagaggggacaacaaggagtat | |
| | gagtactcagtggagtgccaggaggacagtgcctgcccagctgctgaggagagtctgccc | |
| | attgaggtcatggtggatgccgttcacaagctcaagtatgaaaactacaccagcagcttcttc | |
| | atcagggacatcatcaaacctgacccacccaagaacttgcagctgaagccattaaagaattc | |
| | tcggcaggtggaggtcagctgggagtaccctgacacctggagtactccacattcctacttctc | |
| | cctgacattctgcgttcaggtccagggcaagagcaagagagaaaagaaagatagagtcttc | |
| | acggacaagacctcagccacggtcatctgccgcaaaaatgccagcattagcgtgcgggcc | |
| | caggaccgctactatagctcatcttggagcgaatgggcatctgtgccctgcagtggtggcgg | |
| | tggcggcggatctagaaacctccccgtggccactccagacccaggaatgttcccatgccttc | |
| | accactcccaaaacctgctgagggccgtcagcaacatgctccagaaggccagacaaactct | |
| | agaattttacccttgcacttctgaagagattgatcatgaagatatcacaaaagataaaaccagc | |
| | acagtggaggcctgtttaccattggaattaaccaagaatgagagttgcctaaattccagagag | |
| | acctctttcataactaatgggagttgcctggcctccagaaagacctcttttatgatggccctgtg | |
| | ccttagtagtatttatgaagacttgaagatgtaccaggtggagttcaagaccatgaatgcaaa | |
| | gcttctgatggatcctaagaggcagatctttctagatcaaaacatgctggcagttattgatgag | |
| | ctgatgcaggccctgaatttcaacagtgagactgtgccacaaaaatcctcccttgaagaacc | |
| | ggatttttataaaactaaaatcaagctctgcatacttcttcatgctttcagaattcgggcagtgac | |
| | tattgatagagtgatgagctatctgaatgcttcctaaTGATCCGCGGacccaaggtcc | |
| | aactctccaagcggcaatcctctctcgcttcctcagccccactgaatgatcgcgtaaccgtaa | |
| | ttaatctagctacatttaagattaagaaaaaatacgggtagaattggagtgccccaattgtgcc | |
| | aagatggactcatctaggacaattgggctgtactttgattctgcccattcttctagcaacctgtta | |
| | gcatttccgatcgtcctacaagacacaggagatgggaagaagcaaatcgccccgcaatata | |
| | ggatccagcgccttgacttgtggactgatagtaaggaggactcagtattcatcaccacctatg | |
| | gattcatctttcaagttgggaatgaagaagccaccgtcggcatgatcgatgataaacccaag | |
| | cgcgagttactttccgctgcgatgctctgcctaggaagcgtcccaaataccggagaccttatt | |
| | gagctggcaagggcctgtctcactatgatagtcacatgcaagaagagtgcaactaatactga | |
| | gagaatggttttctcagtagtgcaggcaccccaagtgctgcaaagctgtagggttgtggcaa | |
| | acaaatactcatcagtgaatgcagtcaagcacgtgaaagcgccagagaagattcccggga | |
| | gtggaaccctagaatacaaggtgaactttgtctccttgactgtggtaccgaagagggatgtct | |
| | acaagatcccagctgcagtattgaaggtttctggctcgagtctgtacaatcttgcgctcaatgt | |
| | cactattaatgtggaggtagacccgaggagtcctttggttaaatctctgtctaagtctgacagc | |
| | ggatactatgctaacctcttcttgcatattggacttatgaccactgtagataggaaggggaaga | |
| | aagtgacatttgacaagctggaaaagaaaataaggagccttgatctatctgtcgggctcagtg | |
| | atgtgctcgggccttccgtgttggtaaaagcaagaggtgcacggactaagcttttggcacctt | |
| | tcttctctagcagtgggacagcctgctatcccatagcaaatgcttctcctcaggtggccaagat | |
| | actctggagtcaaaccgcgtgcctgcggagcgttaaaatcattatccaagcaggtacccaac | |
| | gcgctgtcgcagtgaccgccgaccacgaggttacctctactaagctggagaaggggcaca | |
| | cccttgccaaatacaatccttttaagaaataagctgcgtctctgagattgcgctccgcccactc | |
| | acccagatcatcatgacacaaaaaactaatctgtcttgattatttacagttagtttacctgtctatc | |
| | aagttagaaaaaacacgggtagaagattctggatcccggttggcgccctccaggtgcaaga | |
| | tgggctccagaccttctaccaagaacccagcacctatgatgctgactatccgggttgcgctg | |
| | gtactgagttgcatctgtccggcaaactccattgatggcaggcctcttgcagctgcaggaatt | |
| | gtggttacaggagacaaagccgtcaacatatacacctcatcccagacaggatcaatcatagt | |
| | taagctcctcccgaatctgcccaaggataaggaggcatgtgcgaaagcccccttggatgcat | |
| | acaacaggacattgaccactttgctcaccccccttggtgactctatccgtaggatacaagagt | |
| | ctgtgactacatctggaggggggagacaggggcgccttataggcgccattattggcggtgt | |
| | ggctcttggggttgcaactgccgcacaaataacagcggccgcagctctgatacaagccaaa | |
| | caaaatgctgccaacatcctccgacttaaagagagcattgccgcaaccaatgaggctgtgca | |
| | tgaggtcactgacggattatcgcaactagcagtggcagttgggaagatgcagcagtttgttaa | |
| | tgaccaatttaataaaacagctcaggaattagactgcatcaaaattgcacagcaagttggtgta | |
| | gagctcaacctgtacctaaccgaattgactacagtattcggaccacaaatcacttcacctgctt | |
| | taaacaagctgactattcaggcactttacaatctagctggtggaaatatggattacttattgact | |
| | aagttaggtgtagggaacaatcaactcagctcattaatcggtagcggcttaatcaccggtaac | |
| | cctattctatacgactcacagactcaactcttgggtatacaggtaactGCCccttcagtcgg | |
| | gaacctaaataatatgcgtgccacctacttggaaaccttatccgtaagcacaaccaggggatt | |
| | tgcctcggcacttgtcccaaaagtggtgacacaggtcggttctgtgatagaagaacttgacac | |
| | ctcatactgtatagaaactgacttagatttatattgtacaagaatagtaacgttccctatgtcccct | |
| | ggtatttattcctgcttgagcggcaatacgtcggcctgtatgtactcaaagaccgaaggcgca | |
| | cttactacaccatacatgactatcaaaggttcagtcatcgccaactgcaagatgacaacatgta | |
| | gatgtgtaaaccccccgggtatcatatcgcaaaactatggagaagccgtgtctctaatagata | |
| | aacaatcatgcaatgttttatccttaggcgggataactttaaggctcagtggggaattcgatgta | |
| | acttatcagaagaatatctcaatacaagattctcaagtaataataacaggcaatcttgatatctc | |
| | aactgagcttgggaatgtcaacaactcgatcagtaatgctttgaataagttagaggaaagcaa | |
| | cagaaaactagacaaagtcaatgtcaaactgactagcacatctgctctcattacctatatcgttt | |
| | tgactatcatatctcttgtttttggtatacttagcctgattctagcatgctacctaatgtacaagcaa | |
| | aaggcgcaacaaaagaccttattatggcttgggaataatactctagatcagatgagagccact | |
| | acaaaaatgtgaacacagatgaggaacgaaggtttccctaatagtaatttgtgtgaaagttctg | |
| | gtagtctgtcagttcagagagttaagaaaaaactaccggttgtagatgaccaaaggacgatat | |
| | acgggtagaacggtaagagaggccgcccctcaattgcgagccaggcttcacaacctccgtt | |
| | ctaccgcttcaccgacaacagtcctcaatcatggaccgcgccgttagccaagttgcgttaga | |
| | gaatgatgaaagagaggcaaaaaatacatggcgcttgatattccggattgcaatcttattctta | |
| | acagtagtgaccttggctatatctgtagcctcccttttatatagcatgggggctagcacaccta | |
| | gcgatcttgtaggcataccgactaggatttccagggcagaagaaaagattacatctacacttg | |
| | gttccaatcaagatgtagtagataggatatataagcaagtggcccttgagtctccgttggcatt | |
| | gttaaatactgagaccacaattatgaacgcaataacatctctctcttatcagattaatggagctg | |
| | caaacaacagtgggtggggggcacctatccatgacccagattatataggggggataggca | |
| | aagaactcattgtagatgatgctagtgatgtcacatcattctatccctctgcatttcaagaacatc | |
| | tgaattttatcccggcgcctactacaggatcaggttgcactcgaataccctcatttgacatgagt | |
| | gctacccattactgctacacccataatgtaatattgtctggatgcagagatcactcacattcata | |
| | tcagtatttagcacttggtgtgctccggacatctgcaacagggagggtattcttttctactctgc | |
| | gttccatcaacctggacgacacccaaaatcggaagtcttgcagtgtgagtgcaactcccctg | |
| | ggttgtgatatgctgtgctcgaaagtcacggagacagaggaagaagattataactcagctgt | |
| | ccctacgcggatggtacatgggaggttagggttcgacggccagtaccacgaaaaggacct | |
| | agatgtcacaacattattcggggactgggtggccaactacccaggagtagggggtggatctt | |
| | ttattgacagccgcgtatggttctcagtctacggagggttaaaacccaattcacccagtgaca | |
| | ctgtacaggaagggaaatatgtgatatacaagcgatacaatgacacatgcccagatgagca | |
| | agactaccagattcgaatggccaagtcttcgtataagcctggacggtttggtgggaaacgca | |
| | tacagcaggctatcttatctatcaaggtgtcaacatccttaggcgaagacccggtactgactgt | |
| | accgcccaacacagtcacactcatgggggccgaaggcagaattctcacagtagggacatct | |
| | catttcttgtatcaacgagggtcatcatacttctctcccgcgttattatatcctatgacagtcagc | |
| | aacaaaacagccactcttcatagtccttatacattcaatgccttcactcggccaggtagtatcc | |
| | cttgccaggcttcagcaagatgccccaactcgtgtgttactggagtctatacagatccatatcc | |
| | cctaatcttctatagaaaccacaccttgcgaggggtattcgggacaatgcttgatggtgtacaa | |
| | gcaagacttaaccctgcgtctgcagtattcgatagcacatcccgcagtcgcattactcgagtg | |
| | agttcaagcagtaccaaagcagcatacacaacatcaacttgttttaaagtggtcaagactaat | |
| | aagacctattgtctcagcattgctgaaatatctaatactctcttcggagaattcagaatcgtccc | |
| | gttactagttgagatcctcaaagatgacggggttagagaagccaggtctggctagttgagtca | |
| | attataaaggagttggaaagatggcattgtatcacctatcttctgcgacatcaagaatcaaacc | |
| | gaatgccggcgcgtgctcgaattccatgttgccagttgaccacaatcagccagtgctcatgc | |
| | gatcagattaagccttgtcaatagtctcttgattaagaaaaaatgtaagtggcaatgagataca | |
| | aggcaaaacagctcatggttaacaatacgggtaggacatggcgagctccggtcctgaaag | |
| | ggcagagcatcagattatcctaccagagtcacacctgtcttcaccattggtcaagcacaaact | |
| | actctattactggaaattaactgggctaccgcttcctgatgaatgtgacttcgaccacctcattct | |
| | cagccgacaatggaaaaaaatacttgaatcggcctctcctgatactgagagaatgataaaac | |
| | tcggaagggcagtacaccaaactcttaaccacaattccagaataaccggagtgctccaccc | |
| | caggtgtttagaagaactggctaatattgaggtcccagattcaaccaacaaatttcggaagatt | |
| | gagaagaagatccaaattcacaacacgagatatggagaactgttcacaaggctgtgtacgc | |
| | atatagagaagaaactgctggggtcatcttggtctaacaatgtcccccggtcagaggagttca | |
| | gcagcattcgtacggatccggcattctggtttcactcaaaatggtccacagccaagtttgcatg | |
| | gctccatataaaacagatccagaggcatctgatggtggcagctaggacaaggtctgcggcc | |
| | aacaaattggtgatgctaacccataaggtaggccaagtctttgtcactcctgaacttgtcgttgt | |
| | gacgcatacgaatgagaacaagttcacatgtcttacccaggaacttgtattgatgtatgcagat | |
| | atgatggagggcagagatatggtcaacataatatcaaccacggcggtgcatctcagaagctt | |
| | atcagagaaaattgatgacattttgcggttaatagacgctctggcaaaagacttgggtaatcaa | |
| | gtctacgatgttgtatcactaatggagggatttgcatacggagctgtccagctactcgagccgt | |
| | caggtacatttgcaggagatttcttcgcattcaacctgcaggagcttaaagacattctaattgg | |
| | cctcctccccaatgatatagcagaatccgtgactcatgcaatcgctactgtattctctggtttag | |
| | aacagaatcaagcagctgagatgttgtgtctgttgcgtctgtggggtcacccactgcttgagt | |
| | cccgtattgcagcaaaggcagtcaggagccaaatgtgcgcaccgaaaatggtagactttga | |
| | tatgatccttcaggtactgtctttcttcaagggaacaatcatcaacgggtacagaaagaagaat | |
| | gcaggtgtgtggccgcgagtcaaagtggatacaatatatgggaaggtcattgggcaactac | |
| | atgcagattcagcagagatttcacacgatatcatgttgagagagtataagagtttatctgcactt | |
| | gaatttgagccatgtatagaatatgaccctgtcaccaacctgagcatgttcctaaaagacaag | |
| | gcaatcgcacaccccaacgataattggcttgcctcgtttaggcggaaccttctctccgaagac | |
| | cagaagaaacatgtaaaagaagcaacttcgactaatcgcctcttgatagagtttttagagtcaa | |
| | atgattttgatccatataaagagatggaatatctgacgacccttgagtaccttagagatgacaat | |
| | gtggcagtatcatactcgctcaaggagaaggaagtgaaagttaatggacggatcttcgctaa | |
| | gctgacaaagaagttaaggaactgtcaggtgatggcggaagggatcctagccgatcagatt | |
| | gcacctttctttcagggaaatggagtcattcaggatagcatatccttgaccaagagtatgctag | |
| | cgatgagtcaactgtcttttaacagcaataagaaacgtatcactgactgtaaagaaagagtatc | |
| | ttcaaaccgcaatcatgatccgaaaagcaagaaccgtcggagagttgcaaccttcataacaa | |
| | ctgacctgcaaaagtactgtcttaattggagatatcagacaatcaaattgttcgctcatgccatc | |
| | aatcagttgatgggcctacctcacttcttcgaatggattcacctaagactgatggacactacga | |
| | tgttcgtaggagaccctttcaatcctccaagtgaccctactgactgtgacctctcaagagtccc | |
| | taatgatgacatatatattgtcagtgccagagggggtatcgaaggattatgccagaagctatg | |
| | gacaatgatctcaattgctgcaatccaacttgctgcagctagatcgcattgtcgtgttgcctgta | |
| | tggtacagggtgataatcaagtaatagcagtaacgagagaggtaagatcagacgactctcc | |
| | ggagatggtgttgacacagttgcatcaagccagtgataatttcttcaaggaattaattcatgtca | |
| | atcatttgattggccataatttgaaggatcgtgaaaccatcaggtcagacacattcttcatatac | |
| | agcaaacgaatcttcaaagatggagcaatcctcagtcaagtcctcaaaaattcatctaaattag | |
| | tgctagtgtcaggtgatctcagtgaaaacaccgtaatgtcctgtgccaacattgcctctactgt | |
| | agcacggctatgcgagaacgggcttcccaaagacttctgttactatttaaactatataatgagtt | |
| | gtgtgcagacatactttgactctgagttctccatcaccaacaattcgcaccccgatcttaatcag | |
| | tcgtggattgaggacatctcttttgtgcactcatatgttctgactcctgcccaattagggggact | |
| | gagtaaccttcaatactcaaggctctacactagaaatatcggtgacccggggactactgctttt | |
| | gcagagatcaagcgactagaagcagtgggattactgagtcctaacattatgactaatatctta | |
| | actaggccgcctgggaatggagattgggccagtctgtgcaacgacccatactctttcaatttt | |
| | gagactgttgcaagcccaaatattgttcttaagaaacatacgcaaagagtcctatttgaaactt | |
| | gttcaaatcccttattgtctggagtgcacacagaggataatgaggcagaagagaaggcattg | |
| | gctgaattcttgcttaatcaagaggtgattcatccccgcgttgcgcatgccatcatggaggca | |
| | agctctgtaggtaggagaaagcaaattcaagggcttgttgacacaacaaacaccgtaattaa | |
| | gattgcgcttactaggaggccattaggcatcaagaggctgatgcggatagtcaattattctag | |
| | catgcatgcaatgctgtttagagacgatgttttttcctccagtagatccaaccaccccttagtctc | |
| | ttctaatatgtgttctctgacactggcagactatgcacggaatagaagctggtcacctttgacg | |
| | ggaggcaggaaaatactgggtgtatctaatcctgatacgatagaactcgtagagggtgagat | |
| | tcttagtgtaagcggagggtgtacaagatgtgacagcggagatgaacaatttacttggttcca | |
| | tcttccaagcaatatagaattgaccgatgacaccagcaagaatcctccgatgagggtaccat | |
| | atctcgggtcaaagacacaggagaggagagctgcctcacttgcaaaaatagctcatatgtcg | |
| | ccacatgtaaaggctgccctaagggcatcatccgtgttgatctgggcttatggggataatgaa | |
| | gtaaattggactgctgctcttacgattgcaaaatctcggtgtaatgtaaacttagagtatcttcg | |
| | gttactgtcccctttacccacggctgggaatcttcaacatagactagatgatggtataactcag | |
| | atgacattcacccctgcatctctctacagggtgtcaccttacattcacatatccaatgattctcaa | |
| | aggctgttcactgaagaaggagtcaaagaggggaatgtggtttaccaacagatcatgctctt | |
| | gggtttatctctaatcgaatcgatctttccaatgacaacaaccaggacatatgatgagatcaca | |
| | ctgcacctacatagtaaatttagttgctgtatcagagaagcacctgttgcggttcctttcgagct | |
| | acttggggtggtaccggaactgaggacagtgacctcaaataagtttatgtatgatcctagccc | |
| | tgtatcggagggagactttgcgagacttgacttagctatcttcaagagttatgagcttaatctgg | |
| | agtcatatcccacgatagagctaatgaacattctttcaatatccagcgggaagttgattggcca | |
| | gtctgtggtttcttatgatgaagatacctccataaagaatgacgccataatagtgtatgacaata | |
| | cccgaaattggatcagtgaagctcagaattcagatgtggtccgcctatttgaatatgcagcact | |
| | tgaagtgctcctcgactgttcttaccaactctattacctgagagtaagaggcctggacaatatt | |
| | gtcttatatatgggtgatttatacaagaatatgccaggaattctactttccaacattgcagctaca | |
| | atatctcatcccgtcattcattcaaggttacatgcagtgggcctggtcaaccatgacggatcac | |
| | accaacttgcagatacggattttatcgaaatgtctgcaaaactattagtatcttgcacccgacgt | |
| | gtgatctccggcttatattcaggaaataagtatgatctgctgttcccatctgtcttagatgataac | |
| | ctgaatgagaagatgcttcagctgatatcccggttatgctgtctgtacacggtactctttgctac | |
| | aacaagagaaatcccgaaaataagaggcttaactgcagaagagaaatgttcaatactcactg | |
| | agtatttactgtcggatgctgtgaaaccattacttagccccgatcaagtgagctctatcatgtct | |
| | cctaacataattacattcccagctaatctgtactacatgtctcggaagagcctcaatttgatcag | |
| | ggaaagggaggacagggatactatcctggcgttgttgttcccccaagagccattattagagtt | |
| | cccttctgtgcaagatattggtgctcgagtgaaagatccattcacccgacaacctgcggcattt | |
| | ttgcaagagttagatttgagtgctccagcaaggtatgacgcattcacacttagtcagattcatc | |
| | ctgaactcacatctccaaatccggaggaagactacttagtacgatacttgttcagagggatag | |
| | ggactgcatcttcctcttggtataaggcatctcatctcctttctgtacccgaggtaagatgtgca | |
| | agacacgggaactccttatacttagctgaagggagcggagccatcatgagtcttctcgaact | |
| | gcatgtaccacatgaaactatctattacaatacgctcttttcaaatgagatgaaccccccgcaa | |
| | cgacatttcgggccgaccccaactcagtttttgaattcggttgtttataggaatctacaggcgg | |
| | aggtaacatgcaaagatggatttgtccaagagttccgtccattatggagagaaaatacagag | |
| | gaaagcgacctgacctcagataaagtagtggggtatattacatctgcagtgccctacagatct | |
| | gtatcattgctgcattgtgacattgaaattcctccagggtccaatcaaagcttactagatcaact | |
| | agctatcaatttatctctgattgccatgcattctgtaagggagggcggggtagtaatcatcaaa | |
| | gtgttgtatgcaatgggatactactttcatctactcatgaacttgtttgctccgtgttccacaaaa | |
| | ggatatattctctctaatggttatgcatgtcgaggagatatggagtgttacctggtatttgtcatg | |
| | ggttacctgggcgggcctacatttgtacatgaggtggtgaggatggcgaaaactctggtgca | |
| | gcggcacggtacgcttttgtctaaatcagatgagatcacactgaccaggttattcacctcaca | |
| | gcggcagcgtgtgacagacatcctatccagtcctttaccaagattaataaagtacttgaggaa | |
| | gaatattgacactgcgctgattgaagccgggggacagcccgtccgtccattctgtgcggag | |
| | agtctggtgagcacgctagcgaacataactcagataacccagatcatcgctagtcacattga | |
| | cacagttatccggtctgtgatatatatggaagctgagggtgatctcgctgacacagtatttctat | |
| | ttaccccttacaatctctctactgacgggaaaaagaggacatcacttaaacagtgcacgagac | |
| | agatcctagaggttacaatactaggtcttagagtcgaaaatctcaataaaataggcgatataat | |
| | cagcctagtgcttaaaggcatgatctccatggaggaccttatcccactaaggacatacttgaa | |
| | gcatagtacctgccctaaatatttgaaggctgtcctaggtattaccaaactcaaagaaatgttta | |
| | cagacacttctgtactgtacttgactcgtgctcaacaaaaattctacatgaaaactataggcaat | |
| | gcagtcaaaggatattacagtaactgtgactcttaacgaaaatcacatattaataggctccttttt | |
| | tggccaattgtattcttgttgatttaatcatattatgttagaaaaaagttgaaccctgactccttag | |
| | gactcgaattcgaactcaaataaatgtcttaaaaaaaggttgcgcacaattattcttgagtgtag | |
| | tctcgtcattcaccaaatctttgtttggt | |

TABLE 11
NDV plasmid sequence

| | | SEQ ID |
|---|---|---|
| Name | Sequence | NO. |
| pT7NDV-LS- | tcttccgcttcctcgctcactgactcgctgcgctcggtcgttcggctgcggcgagcggtatca | 21 |
| L289A plasmid | gctcactcaaaggcggtaatacggttatccacagaatcaggggataacgcaggaaagaac | |
| | atgtgagcaaaaggccagcaaaaggccaggaaccgtaaaaaggccgcgttgctggcgttt | |
| | ttccataggctccgcccccctgacgagcatcacaaaaatcgacgctcaagtcagaggtggc | |
| | gaaacccgacaggactataaagataccaggcgtttccccctggaagctccctcgtgcgctct | |
| | cctgttccgaccctgccgcttaccggatacctgtccgcctttctcccttcgggaagcgtggcg | |
| | ctttctcatagctcacgctgtaggtatctcagttcggtgtaggtcgttcgctccaagctgggctg | |
| | tgtgcacgaaccccccgttcagcccgaccgctgcgccttatccggtaactatcgtcttgagtc | |
| | caacccggtaagacacgacttatcgccactggcagcagccactggtaacaggattagcaga | |
| | gcgaggtatgtaggcggtgctacagagttcttgaagtggtggcctaactacggctacactag | |
| | aagaacagtatttggtatctgcgctctgctgaagccagttaccttcggaaaaagagttggtag | |
| | ctcttgatccggcaaacaaaccaccgctggtagcggtggtttttttgtttgcaagcagcagatt | |
| | acgcgcagaaaaaaaggatctcaagaagatcctttgatcttttctacggggtctgacgctcag | |
| | tggaacgaaaactcacgttaagggattttggtcatgagattatcaaaaaggatcttcacctaga | |
| | tccttttaaattaaaaatgaagttttaaatcaatctaaagtatatatgagtaaacttggtctgacag | |
| | ttaccaatgcttaatcagtgaggcacctatctcagcgatctgtctatttcgttcatccatagttgc | |
| | ctgactccccgtcgtgtagataactacgatacgggagggcttaccatctggccccagtgctg | |
| | caatgataccgcgagacccacgctcaccggctccagatttatcagcaataaaccagccagc | |
| | cggaagggccgagcgcagaagtggtcctgcaactttatccgcctccatccagtctattaattg | |
| | ttgccgggaagctagagtaagtagttcgccagttaatagtttgcgcaacgttgttgccattgct | |
| | acaggcatcgtggtgtcacgctcgtcgtttggtatggcttcattcagctccggttcccaacgat | |
| | caaggcgagttacatgatcccccatgttgtgcaaaaaagcggttagctccttcggtcctccga | |
| | tcgttgtcagaagtaagttggccgcagtgttatcactcatggttatggcagcactgcataattct | |
| | cttactgtcatgccatccgtaagatgcttttctgtgactggtgagtactcaaccaagtcattctga | |
| | gaatagtgtatgcggcgaccgagttgctcttgcccggcgtcaatacgggataataccgcgcc | |
| | acatagcagaactttaaaagtgctcatcattggaaaacgttcttcggggcgaaaactctcaag | |
| | gatcttaccgctgttgagatccagttcgatgtaacccactcgtgcacccaactgatcttcagca | |
| | tcttttactttcaccagcgtttctgggtgagcaaaaacaggaaggcaaaatgccgcaaaaaag | |
| | ggaataagggcgacacggaaatgttgaatactcatactcttcctttttcaatattattgaagcatt | |
| | tatcagggttattgtctcatgagcggatacatatttgaatgtatttagaaaaataaacaaatagg | |
| | ggttccgcgcacatttccccgaaaagtgccacctgacgtctaagaaaccattattatcatgac | |
| | attaacctataaaaataggcgtatcacgaggccctttcgtctcgcgcgtttcggtgatgacggt | |
| | gaaaacctctgacacatgcagctcccggagacggtcacagcttgtctgtaagcggatgccg | |
| | ggagcagacaagcccgtcagggcgcgtcagcgggtgttggcgggtgtcggggctggctt | |
| | aactatgcggcatcagagcagattgtactgagagtgcaccataaaattgtaaacgttaatatttt | |
| | gttaaaattcgcgttaaatttttgttaaatcagctcattttttaaccaataggccgaaatcggcaa | |
| | aatcccttataaatcaaaagaatagcccgagatagggttgagtgttgttccagtttggaacaag | |
| | agtccactattaaagaacgtggactccaacgtcaaagggcgaaaaaccgtctatcagggcg | |
| | atggcccactacgtgaaccatcacccaaatcaagttttttggggtcgaggtgccgtaaagca | |
| | ctaaatcggaaccctaaagggagcccccgatttagagcttgacggggaaagccggcgaac | |
| | gtggcgagaaaggaagggaagaaagcgaaaggagcgggcgctagggcgctggcaagt | |
| | gtagcggtcacgctgcgcgtaaccaccacacccgccgcgcttaatgcgccgctacagggc | |
| | gcgtactatggttgctttgacgtatgcggtgtgaaataccgcacagatgcgtaaggagaaaat | |
| | accgcatcaggcgccattcgccattcaggctgcgcaactgttgggaagggcgatcggtgcg | |
| | ggcctcttcgctattacgccagctggcgaaagggggatgtgctgcaaggcgattaagttgg | |
| | gtaacgccagggttttcccagtcacgacgttgtaaaacgacggccagtgccaagctttaata | |
| | cgactcactatagggaccaaacagagaatccgtgagttacgataaaaggcgaaggagcaa | |
| | ttgaagtcgcacgggtagaaggtgtgaatctcgagtgcgagcccgaagcacaaactcgag | |
| | aaagccttctgccaacatgtcttccgtatttgatgagtacgaacagctcctcgcggctcagact | |
| | cgccccaatggagctcatggagggggagaaaaagggagtaccttaaaagtagacgtcccg | |
| | gtattcactcttaacagtgatgacccagaagatagatggagctttgtggtattctgcctccggat | |
| | tgctgttagcgaagatgccaacaaaccactcaggcaaggtgctctcatatctcttttatgctcc | |
| | cactcacaggtaatgaggaaccatgttgcccttgcagggaaacagaatgaagccacattgg | |
| | ccgtgcttgagattgatggctttgccaacggcacgccccagttcaacaataggagtggagtg | |
| | tctgaagagagagcacagagatttgcgatgatagcaggatctctccctcgggcatgcagca | |
| | acggaaccccgttcgtcacagccggggccgaagatgatgcaccagaagacatcaccgata | |
| | ccctggagaggatcctctctatccaggctcaagtatgggtcacagtagcaaaagccatgact | |
| | gcgtatgagactgcagatgagtcggaaacaaggcgaatcaataagtatatgcagcaaggca | |
| | gggtccaaaagaaatacatcctctaccccgtatgcaggagcacaatccaactcacgatcag | |
| | acagtctcttgcagtccgcatctttttggttagcgagctcaagagaggccgcaacacggcag | |
| | gtggtacctctacttattataacctggtaggggacgtagactcatacatcaggaataccgggc | |
| | ttactgcattcttcttgacactcaagtacggaatcaacaccaagacatcagcccttgcacttagt | |
| | agcctctcaggcgacatccagaagatgaagcagctcatgcgtttgtatcggatgaaaggag | |
| | ataatgcgccgtacatgacattacttggtgatagtgaccagatgagctttgcgcctgccgagt | |
| | atgcacaactttactcctttgccatgggtatggcatcagtcctagataaaggtactgggaaata | |
| | ccaatttgccagggactttatgagcacatcattctggagacttggagtagagtacgctcaggc | |
| | tcagggaagtagcattaacgaggatatggctgccgagctaaagctaaccccagcagcaag | |
| | gaggggcctggcagctgctgcccaacgggtctccgaggagaccagcagcatagacatgc | |
| | ctactcaacaagtcggagtcctcactgggcttagcgagggggggtcccaagctctacaagg | |
| | cggatcgaatagatcgcaagggcaaccagaagccggggatggggagacccaattcctgg | |
| | atctgatgagagcggtagcaaatagcatgagggaggcgccaaactctgcacagggcactc | |
| | cccaatcggggcctcccccaactcctgggccatcccaagataacgacaccgactgggggt | |
| | attgatggacaaaacccagcctgcttccacaaaaacatcccaatgccctcacccgtagtcga | |
| | cccctcgatttgcggctctatatgaccacaccctcaaacaaacatccccctctttcctccctcc | |
| | ccctgctgtacaactacgtacgccctagataccacaggcacaatgcggctcactaacaatca | |
| | aaacagagccgagggaattagaaaaaagtacgggtagaagagggatattcagagatcagg | |
| | gcaagtctcccgagtctctgctctctcctctacctgatagaccaggacaaacatggccaccttt | |
| | acagatgcagagatcgacgagctatttgagacaagtggaactgtcattgacaacataattaca | |
| | gcccagggtaaaccagcagagactgttggaaggagtgcaatcccacaaggcaagaccaa | |
| | ggtgctgagcgcagcatgggagaagcatgggagcatccagccaccggccagtcaagaca | |
| | accccgatcgacaggacagatctgacaaacaaccatccacacccgagcaaacgaccccg | |
| | catgacagcccgccggccacatccgccgaccagccccccacccaggccacagacgaag | |
| | ccgtcgacacacagctcaggaccggagcaagcaactctctgctgttgatgcttgacaagctc | |
| | agcaataaatcgtccaatgctaaaaagggcccatggtcgagcccccaagaggggaatcac | |
| | caacgtccgactcaacagcaggggagtcaacccagtcgcggaaacagtcaggaaagacc | |
| | gcagaaccaagtcaaggccgcccctggaaaccagggcacagacgtgaacacagcatatc | |
| | atggacaatgggaggagtcacaactatcagctggtgcaacccctcatgctctccgatcaagg | |
| | cagagccaagacaatacccttgtatctgcggatcatgtccagccacctgtagactttgtgcaa | |
| | gcgatgatgtctatgatggaggcgatatcacagagagtaagtaaggttgactatcagctagat | |
| | cttgtcttgaaacagacatcctccatccctatgatgcggtccgaaatccaacagctgaaaaca | |
| | tctgttgcagtcatggaagccaacttgggaatgatgaagattctggatcccggttgtgccaac | |
| | atttcatctctgagtgatctacgggcagttgcccgatctcacccggttttagtttcaggccctgg | |
| | agacccctctccctatgtgacacaaggaggcgaaatggcacttaataaactttcgcaaccag | |
| | tgccacatccatctgaattgattaaacccgccactgcatgcgggcctgatataggagtggaa | |
| | aaggacactgtccgtgcattgatcatgtcacgcccaatgcacccgagttcttcagccaagctc | |
| | ctaagcaagttagatgcagccgggtcgatcgaggaaatcaggaaaatcaagcgccttgctc | |
| | taaatggctaattactactgccacacgtagcgggtccctgtccactcggcatcacacggaatc | |
| | tgcaccgagttcccccccgcggacccaaggtccaactctccaagcggcaatcctctctcgct | |
| | tcctcagccccactgaatgatcgcgtaaccgtaattaatctagctacatttaagattaagaaaa | |
| | aatacgggtagaattggagtgccccaattgtgccaagatggactcatctaggacaattgggc | |
| | tgtactttgattctgcccattcttctagcaacctgttagcatttccgatcgtcctacaagacacag | |
| | gagatgggaagaagcaaatcgccccgcaatataggatccagcgccttgacttgtggactga | |
| | tagtaaggaggactcagtattcatcaccacctatggattcatctttcaagttgggaatgaagaa | |
| | gccaccgtcggcatgatcgatgataaacccaagcgcgagttactttccgctgcgatgctctg | |
| | cctaggaagcgtcccaaataccggagaccttattgagctggcaagggcctgtctcactatga | |
| | tagtcacatgcaagaagagtgcaactaatactgagagaatggttttctcagtagtgcaggcac | |
| | cccaagtgctgcaaagctgtagggttgtggcaaacaaatactcatcagtgaatgcagtcaag | |
| | cacgtgaaagcgccagagaagattcccgggagtggaaccctagaatacaaggtgaactttg | |
| | tctccttgactgtggtaccgaagagggatgtctacaagatcccagctgcagtattgaaggtttc | |
| | tggctcgagtctgtacaatcttgcgctcaatgtcactattaatgtggaggtagacccgaggagt | |
| | cctttggttaaatctctgtctaagtctgacagcggatactatgctaacctcttcttgcatattggac | |
| | ttatgaccactgtagataggaaggggaagaaagtgacatttgacaagctggaaaagaaaat | |
| | aaggagccttgatctatctgtcgggctcagtgatgtgctcgggccttccgtgttggtaaaagc | |
| | aagaggtgcacggactaagcttttggcacctttcttctctagcagtgggacagcctgctatcc | |
| | catagcaaatgcttctcctcaggtggccaagatactctggagtcaaaccgcgtgcctgcgga | |
| | gcgttaaaatcattatccaagcaggtacccaacgcgctgtcgcagtgaccgccgaccacga | |
| | ggttacctctactaagctggagaaggggcacacccttgccaaatacaatccttttaagaaata | |
| | agctgcgtctctgagattgcgctccgcccactcacccagatcatcatgacacaaaaaactaat | |
| | ctgtcttgattatttacagttagtttacctgtctatcaagttagaaaaaacacgggtagaagattct | |
| | ggatcccggttggcgccctccaggtgcaagatgggctccagaccttctaccaagaacccag | |
| | cacctatgatgctgactatccgggttgcgctggtactgagttgcatctgtccggcaaactccat | |
| | tgatggcaggcctcttgcagctgcaggaattgtggttacaggagacaaagccgtcaacatat | |
| | acacctcatcccagacaggatcaatcatagttaagctcctcccgaatctgcccaaggataag | |
| | gaggcatgtgcgaaagcccccttggatgcatacaacaggacattgaccactttgctcacccc | |
| | ccttggtgactctatccgtaggatacaagagtctgtgactacatctggaggggggagacagg | |
| | ggcgccttataggcgccattattggcggtgtggctcttggggttgcaactgccgcacaaataa | |
| | cagcggccgcagctctgatacaagccaaacaaaatgctgccaacatcctccgacttaaaga | |
| | gagcattgccgcaaccaatgaggctgtgcatgaggtcactgacggattatcgcaactagca | |
| | gtggcagttgggaagatgcagcagtttgttaatgaccaatttaataaaacagctcaggaatta | |
| | gactgcatcaaaattgcacagcaagttggtgtagagctcaacctgtacctaaccgaattgact | |
| | acagtattcggaccacaaatcacttcacctgctttaaacaagctgactattcaggcactttaca | |
| | atctagctggtggaaatatggattacttattgactaagttaggtgtagggaacaatcaactcag | |
| | ctcattaatcggtagcggcttaatcaccggtaaccctattctatacgactcacagactcaactct | |
| | tgggtatacaggtaactgccccttcagtcgggaacctaaataatatgcgtgccacctacttgg | |
| | aaaccttatccgtaagcacaaccaggggatttgcctcggcacttgtcccaaaagtggtgaca | |
| | caggtcggttctgtgatagaagaacttgacacctcatactgtatagaaactgacttagatttata | |
| | ttgtacaagaatagtaacgttccctatgtcccctggtatttattcctgcttgagcggcaatacgtc | |
| | ggcctgtatgtactcaaagaccgaaggcgcacttactacaccatacatgactatcaaaggttc | |
| | agtcatcgccaactgcaagatgacaacatgtagatgtgtaaaccccccgggtatcatatcgc | |
| | aaaactatggagaagccgtgtctctaatagataaacaatcatgcaatgttttatccttaggcgg | |
| | gataactttaaggctcagtggggaattcgatgtaacttatcagaagaatatctcaatacaagatt | |
| | ctcaagtaataataacaggcaatcttgatatctcaactgagcttgggaatgtcaacaactcgat | |
| | cagtaatgctttgaataagttagaggaaagcaacagaaaactagacaaagtcaatgtcaaac | |
| | tgactagcacatctgctctcattacctatatcgttttgactatcatatctcttgtttttggtatacttag | |
| | cctgattctagcatgctacctaatgtacaagcaaaaggcgcaacaaaagaccttattatggctt | |
| | gggaataatactctagatcagatgagagccactacaaaaatgtgaacacagatgaggaacg | |
| | aaggtttccctaatagtaatttgtgtgaaagttctggtagtctgtcagttcagagagttaagaaa | |
| | aaactaccggttgtagatgaccaaaggacgatatacgggtagaacggtaagagaggccgc | |
| | ccctcaattgcgagccaggcttcacaacctccgttctaccgcttcaccgacaacagtcctcaa | |
| | tcatggaccgcgccgttagccaagttgcgttagagaatgatgaaagagaggcaaaaaatac | |
| | atggcgcttgatattccggattgcaatcttattcttaacagtagtgaccttggctatatctgtagc | |
| | ctcccttttatatagcatgggggctagcacacctagcgatcttgtaggcataccgactaggatt | |
| | tccagggcagaagaaaagattacatctacacttggttccaatcaagatgtagtagataggata | |
| | tataagcaagtggcccttgagtctccgttggcattgttaaatactgagaccacaattatgaacg | |
| | caataacatctctctcttatcagattaatggagctgcaaacaacagtgggtggggggcaccta | |
| | tccatgacccagattatataggggggataggcaaagaactcattgtagatgatgctagtgatg | |
| | tcacatcattctatccctctgcatttcaagaacatctgaattttatcccggcgcctactacaggat | |
| | caggttgcactcgaataccctcatttgacatgagtgctacccattactgctacacccataatgta | |
| | atattgtctggatgcagagatcactcacattcatatcagtatttagcacttggtgtgctccggac | |
| | atctgcaacagggagggtattcttttctactctgcgttccatcaacctggacgacacccaaaat | |
| | cggaagtcttgcagtgtgagtgcaactcccctgggttgtgatatgctgtgctcgaaagtcacg | |
| | gagacagaggaagaagattataactcagctgtccctacgcggatggtacatgggaggttag | |
| | ggttcgacggccagtaccacgaaaaggacctagatgtcacaacattattcggggactgggt | |
| | ggccaactacccaggagtagggggtggatcttttattgacagccgcgtatggttctcagtcta | |
| | cggagggttaaaacccaattcacccagtgacactgtacaggaagggaaatatgtgatataca | |
| | agcgatacaatgacacatgcccagatgagcaagactaccagattcgaatggccaagtcttc | |
| | gtataagcctggacggtttggtgggaaacgcatacagcaggctatcttatctatcaaggtgtc | |
| | aacatccttaggcgaagacccggtactgactgtaccgcccaacacagtcacactcatgggg | |
| | gccgaaggcagaattctcacagtagggacatctcatttcttgtatcaacgagggtcatcatact | |
| | tctctcccgcgttattatatcctatgacagtcagcaacaaaacagccactcttcatagtccttata | |
| | cattcaatgccttcactcggccaggtagtatcccttgccaggcttcagcaagatgccccaact | |
| | cgtgtgttactggagtctatacagatccatatcccctaatcttctatagaaaccacaccttgcga | |
| | ggggtattcgggacaatgcttgatggtgtacaagcaagacttaaccctgcgtctgcagtattc | |
| | gatagcacatcccgcagtcgcattactcgagtgagttcaagcagtaccaaagcagcatacac | |
| | aacatcaacttgttttaaagtggtcaagactaataagacctattgtctcagcattgctgaaatatc | |
| | taatactctcttcggagaattcagaatcgtcccgttactagttgagatcctcaaagatgacggg | |
| | gttagagaagccaggtctggctagttgagtcaattataaaggagttggaaagatggcattgta | |
| | tcacctatcttctgcgacatcaagaatcaaaccgaatgccggcgcgtgctcgaattccatgtt | |
| | gccagttgaccacaatcagccagtgctcatgcgatcagattaagccttgtcaatagtctcttga | |
| | ttaagaaaaaatgtaagtggcaatgagatacaaggcaaaacagctcatggttaacaatacgg | |
| | gtaggacatggcgagctccggtcctgaaagggcagagcatcagattatcctaccagagtca | |
| | cacctgtcttcaccattggtcaagcacaaactactctattactggaaattaactgggctaccgct | |
| | tcctgatgaatgtgacttcgaccacctcattctcagccgacaatggaaaaaaatacttgaatcg | |
| | gcctctcctgatactgagagaatgataaaactcggaagggcagtacaccaaactcttaacca | |
| | caattccagaataaccggagtgctccaccccaggtgtttagaagaactggctaatattgaggt | |
| | cccagattcaaccaacaaatttcggaagattgagaagaagatccaaattcacaacacgagat | |
| | atggagaactgttcacaaggctgtgtacgcatatagagaagaaactgctggggtcatcttggt | |
| | ctaacaatgtcccccggtcagaggagttcagcagcattcgtacggatccggcattctggtttc | |
| | actcaaaatggtccacagccaagtttgcatggctccatataaaacagatccagaggcatctg | |
| | atggtggcagctaggacaaggtctgcggccaacaaattggtgatgctaacccataaggtag | |
| | gccaagtctttgtcactcctgaacttgtcgttgtgacgcatacgaatgagaacaagttcacatg | |
| | tcttacccaggaacttgtattgatgtatgcagatatgatggagggcagagatatggtcaacata | |
| | atatcaaccacggcggtgcatctcagaagcttatcagagaaaattgatgacattttgcggttaa | |
| | tagacgctctggcaaaagacttgggtaatcaagtctacgatgttgtatcactaatggagggatt | |
| | tgcatacggagctgtccagctactcgagccgtcaggtacatttgcaggagatttcttcgcattc | |
| | aacctgcaggagcttaaagacattctaattggcctcctccccaatgatatagcagaatccgtg | |
| | actcatgcaatcgctactgtattctctggtttagaacagaatcaagcagctgagatgttgtgtct | |
| | gttgcgtctgtggggtcacccactgcttgagtcccgtattgcagcaaaggcagtcaggagcc | |
| | aaatgtgcgcaccgaaaatggtagactttgatatgatccttcaggtactgtctttcttcaaggga | |
| | acaatcatcaacgggtacagaaagaagaatgcaggtgtgtggccgcgagtcaaagtggat | |
| | acaatatatgggaaggtcattgggcaactacatgcagattcagcagagatttcacacgatatc | |
| | atgttgagagagtataagagtttatctgcacttgaatttgagccatgtatagaatatgaccctgt | |
| | caccaacctgagcatgttcctaaaagacaaggcaatcgcacaccccaacgataattggcttg | |
| | cctcgtttaggcggaaccttctctccgaagaccagaagaaacatgtaaaagaagcaacttcg | |
| | actaatcgcctcttgatagagtttttagagtcaaatgattttgatccatataaagagatggaatat | |
| | ctgacgacccttgagtaccttagagatgacaatgtggcagtatcatactcgctcaaggagaa | |
| | ggaagtgaaagttaatggacggatcttcgctaagctgacaaagaagttaaggaactgtcagg | |
| | tgatggcggaagggatcctagccgatcagattgcacctttctttcagggaaatggagtcattc | |
| | aggatagcatatccttgaccaagagtatgctagcgatgagtcaactgtcttttaacagcaataa | |
| | gaaacgtatcactgactgtaaagaaagagtatcttcaaaccgcaatcatgatccgaaaagca | |
| | agaaccgtcggagagttgcaaccttcataacaactgacctgcaaaagtactgtcttaattgga | |
| | gatatcagacaatcaaattgttcgctcatgccatcaatcagttgatgggcctacctcacttcttc | |
| | gaatggattcacctaagactgatggacactacgatgttcgtaggagaccctttcaatcctcca | |
| | agtgaccctactgactgtgacctctcaagagtccctaatgatgacatatatattgtcagtgcca | |
| | gagggggtatcgaaggattatgccagaagctatggacaatgatctcaattgctgcaatccaa | |
| | cttgctgcagctagatcgcattgtcgtgttgcctgtatggtacagggtgataatcaagtaatag | |
| | cagtaacgagagaggtaagatcagacgactctccggagatggtgttgacacagttgcatca | |
| | agccagtgataatttcttcaaggaattaattcatgtcaatcatttgattggccataatttgaaggat | |
| | cgtgaaaccatcaggtcagacacattcttcatatacagcaaacgaatcttcaaagatggagca | |
| | atcctcagtcaagtcctcaaaaattcatctaaattagtgctagtgtcaggtgatctcagtgaaaa | |
| | caccgtaatgtcctgtgccaacattgcctctactgtagcacggctatgcgagaacgggcttcc | |
| | caaagacttctgttactatttaaactatataatgagttgtgtgcagacatactttgactctgagttc | |
| | tccatcaccaacaattcgcaccccgatcttaatcagtcgtggattgaggacatctcttttgtgca | |
| | ctcatatgttctgactcctgcccaattagggggactgagtaaccttcaatactcaaggctctac | |
| | actagaaatatcggtgacccggggactactgcttttgcagagatcaagcgactagaagcagt | |
| | gggattactgagtcctaacattatgactaatatcttaactaggccgcctgggaatggagattgg | |
| | gccagtctgtgcaacgacccatactctttcaattttgagactgttgcaagcccaaatattgttctt | |
| | aagaaacatacgcaaagagtcctatttgaaacttgttcaaatcccttattgtctggagtgcaca | |
| | cagaggataatgaggcagaagagaaggcattggctgaattcttgcttaatcaagaggtgatt | |
| | catccccgcgttgcgcatgccatcatggaggcaagctctgtaggtaggagaaagcaaattc | |
| | aagggcttgttgacacaacaaacaccgtaattaagattgcgcttactaggaggccattaggc | |
| | atcaagaggctgatgcggatagtcaattattctagcatgcatgcaatgctgtttagagacgatg | |
| | ttttttcctccagtagatccaaccaccccttagtctcttctaatatgtgttctctgacactggcaga | |
| | ctatgcacggaatagaagctggtcacctttgacgggaggcaggaaaatactgggtgtatcta | |
| | atcctgatacgatagaactcgtagagggtgagattcttagtgtaagcggagggtgtacaagat | |
| | gtgacagcggagatgaacaatttacttggttccatcttccaagcaatatagaattgaccgatga | |
| | caccagcaagaatcctccgatgagggtaccatatctcgggtcaaagacacaggagaggag | |
| | agctgcctcacttgcaaaaatagctcatatgtcgccacatgtaaaggctgccctaagggcatc | |
| | atccgtgttgatctgggcttatggggataatgaagtaaattggactgctgctcttacgattgcaa | |
| | aatctcggtgtaatgtaaacttagagtatcttcggttactgtcccctttacccacggctgggaat | |
| | cttcaacatagactagatgatggtataactcagatgacattcacccctgcatctctctacaggg | |
| | tgtcaccttacattcacatatccaatgattctcaaaggctgttcactgaagaaggagtcaaaga | |
| | ggggaatgtggtttaccaacagatcatgctcttgggtttatctctaatcgaatcgatctttccaat | |
| | gacaacaaccaggacatatgatgagatcacactgcacctacatagtaaatttagttgctgtatc | |
| | agagaagcacctgttgcggttcctttcgagctacttggggtggtaccggaactgaggacagt | |
| | gacctcaaataagtttatgtatgatcctagccctgtatcggagggagactttgcgagacttgac | |
| | ttagctatcttcaagagttatgagcttaatctggagtcatatcccacgatagagctaatgaacat | |
| | tctttcaatatccagcgggaagttgattggccagtctgtggtttcttatgatgaagatacctccat | |
| | aaagaatgacgccataatagtgtatgacaatacccgaaattggatcagtgaagctcagaattc | |
| | agatgtggtccgcctatttgaatatgcagcacttgaagtgctcctcgactgttcttaccaactct | |
| | attacctgagagtaagaggcctggacaatattgtcttatatatgggtgatttatacaagaatatg | |
| | ccaggaattctactttccaacattgcagctacaatatctcatcccgtcattcattcaaggttacat | |
| | gcagtgggcctggtcaaccatgacggatcacaccaacttgcagatacggattttatcgaaat | |
| | gtctgcaaaactattagtatcttgcacccgacgtgtgatctccggcttatattcaggaaataagt | |
| | atgatctgctgttcccatctgtcttagatgataacctgaatgagaagatgcttcagctgatatcc | |
| | cggttatgctgtctgtacacggtactctttgctacaacaagagaaatcccgaaaataagaggc | |
| | ttaactgcagaagagaaatgttcaatactcactgagtatttactgtcggatgctgtgaaaccatt | |
| | acttagccccgatcaagtgagctctatcatgtctcctaacataattacattcccagctaatctgta | |
| | ctacatgtctcggaagagcctcaatttgatcagggaaagggaggacagggatactatcctgg | |
| | cgttgttgttcccccaagagccattattagagttcccttctgtgcaagatattggtgctcgagtg | |
| | aaagatccattcacccgacaacctgcggcatttttgcaagagttagatttgagtgctccagca | |
| | aggtatgacgcattcacacttagtcagattcatcctgaactcacatctccaaatccggaggaa | |
| | gactacttagtacgatacttgttcagagggatagggactgcatcttcctcttggtataaggcatc | |
| | tcatctcctttctgtacccgaggtaagatgtgcaagacacgggaactccttatacttagctgaa | |
| | gggagcggagccatcatgagtcttctcgaactgcatgtaccacatgaaactatctattacaat | |
| | acgctcttttcaaatgagatgaaccccccgcaacgacatttcgggccgaccccaactcagttt | |
| | ttgaattcggttgtttataggaatctacaggcggaggtaacatgcaaagatggatttgtccaag | |
| | agttccgtccattatggagagaaaatacagaggaaagcgacctgacctcagataaagtagtg | |
| | gggtatattacatctgcagtgccctacagatctgtatcattgctgcattgtgacattgaaattcct | |
| | ccagggtccaatcaaagcttactagatcaactagctatcaatttatctctgattgccatgcattct | |
| | gtaagggagggcggggtagtaatcatcaaagtgttgtatgcaatgggatactactttcatcta | |
| | ctcatgaacttgtttgctccgtgttccacaaaaggatatattctctctaatggttatgcatgtcga | |
| | ggagatatggagtgttacctggtatttgtcatgggttacctgggcgggcctacatttgtacatg | |
| | aggtggtgaggatggcgaaaactctggtgcagcggcacggtacgcttttgtctaaatcagat | |
| | gagatcacactgaccaggttattcacctcacagcggcagcgtgtgacagacatcctatccag | |
| | tcctttaccaagattaataaagtacttgaggaagaatattgacactgcgctgattgaagccggg | |
| | ggacagcccgtccgtccattctgtgcggagagtctggtgagcacgctagcgaacataactc | |
| | agataacccagatcatcgctagtcacattgacacagttatccggtctgtgatatatatggaagc | |
| | tgagggtgatctcgctgacacagtatttctatttaccccttacaatctctctactgacgggaaaa | |
| | agaggacatcacttaaacagtgcacgagacagatcctagaggttacaatactaggtcttaga | |
| | gtcgaaaatctcaataaaataggcgatataatcagcctagtgcttaaaggcatgatctccatg | |
| | gaggaccttatcccactaaggacatacttgaagcatagtacctgccctaaatatttgaaggct | |
| | gtcctaggtattaccaaactcaaagaaatgtttacagacacttctgtactgtacttgactcgtgct | |
| | caacaaaaattctacatgaaaactataggcaatgcagtcaaaggatattacagtaactgtgac | |
| | tcttaacgaaaatcacatattaataggctccttttttggccaattgtattcttgttgatttaatcatatt | |
| | atgttagaaaaaagttgaaccctgactccttaggactcgaattcgaactcaaataaatgtcttaa | |
| | aaaaaggttgcgcacaattattcttgagtgtagtctcgtcattcaccaaatctttgtttggtgcgc | |
| | gcggccggcatggtcccagcctcctcgctggcgccggctgggcaacattccgaggggac | |
| | cgtcccctcggtaatggcgaatgggacgtcgactgctaacaaagcccgaaaggaagctga | |
| | gttggctgctgccaccgctgagcaataactagcataaccccttggggcctctaaacgggtctt | |
| | gaggggttttttgctgaaaggaggaactatatgcgcgcagatctgtcatgatgatcattgcaat | |
| | tggatccatatatagggcccgggttataattacctcaggtcgacgtcccatggccattcgaatt | |
| | cgtaatcatggtcatagctgtttcctgtgtgaaattgttatccgctcacaattccacacaacatac | |
| | gagccggaagcataaagtgtaaagcctggggtgcctaatgagtgagctaactcacattaatt | |
| | gcgttgcgctcactgcccgctttccagtcgggaaacctgtcgtgccagctgcattaatgaatc | |
| | ggccaacgcgcggggagaggcggtttgcgtattgggcgc | |

TABLE 12
Exemplary antibody-related sequences

| | | SEQ ID |
|---|---|---|
| Name | Sequence | NO. |
| Pembrolizumab | RASKGVSTSGYSYLH | 1 |
| Light Chain | | |
| CDR1 | | |
| Pembrolizumab | LASYLES | 2 |
| Light Chain | | |
| CDR2 | | |
| Pembrolizumab | QHSRDLPLT | 3 |
| Light Chain | | |
| CDR3 | | |
| Pembrolizumab | EIVLTQSPATLSLSPGERATLSCRASKGVSTSGYSYLHWYQ | 4 |
| Light Chain | QKPGQAPRLLIYLASYLESGVPARFSGSGSGTDFTLTISSLE | |
| Variable Region | PEDFAVYYCQHSRDLPLTFGGGTKVEIK | |
| Pembrolizumab | EIVLTQSPATLSLSPGERATLSCRASKGVSTSGYSYLHWYQ | 5 |
| Light Chain | QKPGQAPRLLIYLASYLESGVPARFSGSGSGTDFTLTISSLE | |
| | PEDFAVYYCQHSRDLPLTFGGGTKVEIKRTVAAPSVFIFPP | |
| | SDEQLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |
| Pembrolizumab | NYYMY | 6 |
| Heavy Chain | | |
| CDR1 | | |
| Pembrolizumab | GINPSNGGTNFNEKFKN | 7 |
| Heavy Chain | | |
| CDR2 | | |
| Pembrolizumab | RDYRFDMGFDY | 8 |
| Heavy Chain | | |
| CDR3 | | |
| Pembrolizumab | QVQLVQSGVEVKKPGASVKVSCKASGYTFTNYYMYWVR | 9 |
| Heavy Chain | QAPGQGLEWMGGINPSNGGTNFNEKFKNRVTLTTDSSTTT | |
| Variable Region | AYMELKSLQFDDTAVYYCARRDYRFDMGFDYWGQGTTV | |
| | TVSS | |
| Pembrolizumab | QVQLVQSGVEVKKPGASVKVSCKASGYTFTNYYMYWVR | 10 |
| Heavy Chain | QAPGQGLEWMGGINPSNGGTNFNEKFKNRVTLTTDSSTTT | |
| | AYMELKSLQFDDTAVYYCARRDYRFDMGFDYWGQGTTV | |
| | TVSSASTKGPSVFPLAPCSRSTSESTAALGCLVKDYFPEPV | |
| | TVSWNSGALTSGVHTFPAVLQSSGLYSLSSVVTVPSSSLGT | |
| | KTYTCNVDHKPSNTKVDKRVESKYGPPCPPCPAPEFLGGP | |
| | SVFLFPPKPKDTLMISRTPEVTCVVVDVSQEDPEVQFNWY | |
| | VDGVEVHNAKTKPREEQFNSTYRVVSVLTVLHQDWLNG | |
| | KEYKCKVSNKGLPSSIEKTISKAKGQPREPQVYTLPPSQEE | |
| | MTKNQVSLTCLVKGFYPSDIAVEWESNGQPENNYKTTPP | |
| | VLDSDGSFFLYSRLTVDKSRWQEGNVFSCSVMHEALHNH | |
| | YTQKSLSLSLGK | |
| Nivolumab Light | RASQSVSSYLA | 11 |
| Chain CDR1 | | |
| Nivolumab Light | DASNRAT | 12 |
| Chain CDR2 | | |
| Nivolumab Light | QQSSNWPRT | 13 |
| Chain CDR3 | | |
| Nivolumab Light | EIVLTQSPATLSLSPGERATLSCRASQSVSSYLAWYQQKPG | 14 |
| Chain Variable | QAPRLLIYDASNRATGIPARFSGSGSGTDFTLTISSLEPEDF | |
| Region | AVYYCQQSSNWPRTFGQGTKVEIK | |
| Nivolumab Light | EIVLTQSPATLSLSPGERATLSCRASQSVSSYLAWYQQKPG | 15 |
| Chain | QAPRLLIYDASNRATGIPARFSGSGSGTDFTLTISSLEPEDF | |
| | AVYYCQQSSNWPRTFGQGTKVEIKRTVAAPSVFIFPPSDE | |
| | QLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| Nivolumab | NSGMH | 16 |
| Heavy Chain | | |
| CDR1 | | |
| Nivolumab | VIWYDGSKRYYADSVKG | 17 |
| Heavy Chain | | |
| CDR2 | | |
| Nivolumab | NDDY | 18 |
| Heavy Chain | | |
| CDR3 | | |
| Nivolumab | QVQLVESGGGVVQPGRSLRLDCKASGITFSNSGMHWVRQ | 19 |
| Heavy Chain | APGKGLEWVAVIWYDGSKRYYADSVKGRFTISRDNSKNT | |
| Variable Region | LFLQMNSLRAEDTAVYYCATNDDYWGQGTLVTVSS | |
| Nivolumab | QVQLVESGGGVVQPGRSLRLDCKASGITFSNSGMHWVRQ | 20 |
| Heavy Chain | APGKGLEWVAVIWYDGSKRYYADSVKGRFTISRDNSKNT | |
| | LFLQMNSLRAEDTAVYYCATNDDYWGQGTLVTVSSAST | |
| | KGPSVFPLAPCSRSTSESTAALGCLVKDYFPEPVTVSWNSG | |
| | ALTSGVHTFPAVLQSSGLYSLSSVVTVPSSSLGTKTYTCNV | |
| | DHKPSNTKVDKRVESKYGPPCPPCPAPEFLGGPSVFLFPPK | |
| | PKDTLMISRTPEVTCVVVDVSQEDPEVQFNWYVDGVEVH | |
| | NAKTKPREEQFNSTYRVVSVLTVLHQDWLNGKEYKCKVS | |
| | NKGLPSSIEKTISKAKGQPREPQVYTLPPSQEEMTKNQVSL | |
| | TCLVKGFYPSDIAVEWESNGQPENNYKTTPPVLDSDGSFF | |
| | LYSRLTVDKSRWQEGNVFSCSVMHEALHNHYTQKSLSLS | |
| | LGK | |

TABLE 13
Primer sequences

| | | SEQ ID |
|---|---|---|
| Name | Sequence | NO. |
| Human IL-12 | GCACCGAGTTCCCCCCCGCGGTTAGAAA | 32 |
| Forward primer | AAATACGGGTAGAACCGCCACCATGGGT | |
| | CACCAGCAG | |
| Human IL-12 | AGTTGGACCTTGGGTCCGCGGATTAGGA | 33 |
| Reverse primer | AGCATTCAG | |
| muIL-2 Forward | GCACCGAGTTCCCCCCCGCGGTTAGAAA | 34 |
| Primer | AAATACGGGTAGAACCGCCACCATGTACA | |
| | GCATGCAG | |
| muIL-2 Reverse | AGTTGGACCTTGGGTCCGCGGATTATTG | 35 |
| Primer | AGGGCTTGT | |
| Primer | TCGATCGAGGAAATCAGG | 36 |
| Primer | GTACAGCCCAATTGTCC | 37 |

TABLE 15
Cleavage sequences

| | | SEQ ID |
|---|---|---|
| Name | Sequence | NO. |
| S116 F protein | HNRTKSF | 56 |
| cleavage site | | |
| S116K F protein | HNKTKSF | 58 |
| cleavage site | | |
| S116m F protein | HNRMKSF | 69 |
| cleavage site | | |
| S116KMF | HNKMSFI | 70 |
| protein cleavage | | |
| site | | |
| R116 F protein | HNRTKRFI | 71 |
| cleavage site | | |
| LaSota strain F | GRQGRL | 72 |
| protein cleavage | | |
| site | | |

### 6. EXAMPLES

The working examples of International Patent Application Publication No. WO 2014/158811 (i.e., Sections 6 and 7 of International Patent Application Publication No. WO 2014/158811) and U.S. Patent Application Publication Nos. 2016/0015760 A1 (i.e., Section 6 of U.S. Patent Application Publication No. 2016/0015760 A1) and 2014/0271677 A1 (i.e., Sections 6 and 7 of U.S. Patent Application Publication No. 2014/0271677 A1) are incorporated by reference herein in their entireties.

### 6.1 Example 1

This example demonstrates the therapeutic efficacy of NDV therapy in combination with immune checkpoint modulators that are immunostimulatory in the treatment of cancer.

### 6.1.1 Materials & Methods

### 6.1.1.1 Mice

BALB/c mice (6-8 weeks old), and WT C57BL/6 mice were purchased from Jackson Laboratory. All mice were maintained in microisolator cages and treated in accordance with the NIH and American Association of Laboratory Animal Care regulations. All mouse procedures and experiments for this study were approved by the Memorial Sloan-Kettering Cancer Center Institutional Animal Care and Use Committee.

### 6.1.1.2 Cell Lines

The murine cancer cell lines for melanoma (B16F10), and colon carcinoma (CT26 and MC38) were maintained in RPMI medium supplemented with 10% fetal calf serum and penicillin with streptomycin. The murine prostate cancer cell line TRAMP-C2 was maintained in DMEM medium supplemented with 5% fetal calf serum (FCS; Mediatech, Inc.), 5% Nu Serum IV (BD Biosciences) HEPES, 2-ME, pen/strep, L-glut, 5 μg/mL insulin (Sigma), and 10 nmol/L DHT (Sigma).

### 6.1.1.3 Antibodies

Therapeutic anti-PD-1 (clone RMP1-14) and anti-PD-L1 monoclonal antibodies were produced by BioXcell. Antibodies used for flow cytometry were purchased from eBioscience, Biolegend, Invitrogen, and BD Pharmingen.

### 6.1.1.4 Viruses and Cloning

Recombinant lentogenic NDV LaSota strain was used for all experiments. Viruses were rescued from cDNA using methods described previously and sequenced by reverse transcription PCR for insert fidelity. Virus titers were determined by serial dilution and immunofluorescence in Vero cells.

### 6.1.1.5 In Vitro Infection Experiments

For evaluation of upregulation of surface MHC-I, MHC-II, and ICAM-1 by NDV, and for evaluation of surface expression of the ICOSL transgene from the NDV-ICOSL virus, B16F10 cells were infected in 6-well dishes at MOI 2 in triplicate. Twenty-four hours later, the cells were harvested by mechanical scraping and processed for surface labeling and quantification by flow cytometry. For virus growth curve experiments, B16F10 cells were incubated at room temperature with the virus in 6-well culture dishes at the indicated MOIs in a total volume of 100 μl. One hour after the incubation, the infection media was aspirated and the cells were incubated at 37° C. in 1 ml of DMEM with 10% chick allantoic fluid. After 24, 48, and 72 hours, the supernatants were collected and virus titers were determined as above. For in vitro cytotoxicity experiments, the infections were carried out in a similar fashion. At 24, 48, 72, and 96 hours post infection the cells were washed and incubated with 1% Triton X-100 at 37° C. for 30 minutes. LDH activity in the lysates was determined using the Promega CytoTox 96 assay kit, according to the manufacturer's instructions.

### 6.1.1.6 Tumor Challenge Survival Experiments

Bilateral flank tumor models were established to monitor for therapeutic efficacy in both injected and systemic tumors. Treatment schedules and cell doses were established for each tumor model to achieve 10-20% tumor clearance by NDV or anti-PD-1 as single agents. For experiments evaluating combination therapy of wild-type NDV (NDV-WT) with immune checkpoint blockade, B16F10 tumors were implanted by injection of 2×10⁵ B16F10 cells in the right flank i.d. on day 0 and 5×10⁴ cells in the left flank on day 4. On days 7, 10, 13, and 16 the mice were treated with 4 intratumoral injections of 2×10⁷ pfu of NDV in PBS in a total volume of 100 μl. Concurrently, on days 7, 10, 13, and 16 the mice received 4 i.p. injections of anti-PD-1 antibody (250 μg). Control groups received a corresponding dose of isotype antibody i.p. and intratumoral injection of PBS. Tumor size and incidence were monitored over time by measurement with a caliper.

For the TRAMP-C2 model, 5×10⁵ cells were implanted in right flank on day 0 and 5×10⁵ cells were implanted in the left flank on day 8. Treatment was performed on days 11, 14, 17, and 20 in the similar fashion to above.

For the CT26 model, tumors were implanted by injection of 1×10⁶ CT26 cells in the right flank i.d. on day 0 and 1×10⁶ cells in the left flank on day 2. Treatment was carried out as above on days 6, 9, and 12.

### 6.1.1.7 Isolation of Tumor-Infiltrating Lymphocytes

B16F10 tumors were implanted by injection of 2×10⁵ B16F10 cells in the right flank i.d. on day 0 and 2×10⁵ cells in the left flank on day 4. On days 7, 10, and 13 the mice were treated with 3 intratumoral injections of 2×10⁷ pfu of NDV, and 250 μg of i.p. anti-PD-1 antibody, where specified. On day 15, mice were sacrificed by CO₂ inhalation. Tumors and tumor-draining lymph nodes were removed using forceps and surgical scissors and weighed. Tumors from each group were minced with scissors prior to incubation with 1.67 Wünsch U/mL Liberase and 0.2 mg/mL DNase for 30 minutes at 37° C. Tumors were homogenized by repeated pipetting and filtered through a 70-μm nylon filter. Cell suspensions were washed once with complete RPMI and purified on a Ficoll gradient to eliminate dead cells. Cells from tumor draining lymph nodes were isolated by grinding the lymph nodes through a 70-μm nylon filter.

### 6.1.1.8 Flow Cytometry

Cells isolated from tumors or tumor-draining lymph nodes were processed for surface labeling with several antibody panels staining CD45, CD3, CD4, CD8, CD44, PD-1, ICOS, CD11c, CD19, NK1.1, CD11b, F4/80, Ly6C and Ly6G. Fixable viability dye eFluor780 (eBioscience) was used to distinguish the live cells. Cells were further permeabilized using FoxP3 fixation and permeabilization kit (eBioscience) and stained for Ki-67, FoxP3, Granzyme B, CTLA-4, and IFN gamma. Data was acquired using the LSRII Flow cytometer (BD Biosciences) and analyzed using FlowJo software (Treestar).

### 6.1.1.9 DC Purification and Loading

Spleens from naïve mice were isolated and digested with 1.67 Wünsch U/mL Liberase and 0.2 mg/mL DNase for 30 minutes at 37° C. The resulting cell suspensions were filtered through 70 μm nylon filter and washed once with complete RPMI. CD11c+ dendritic cells were purified by positive selection using Miltenyi magnetic beads. Isolated dendritic cells were cultured overnight with recombinant GM-CSF and B16F10 tumor lysates and were purified on Ficoll gradient.

### 6.1.1.10 Analysis of Cytokine Production

Cell suspensions from tumors or tumor-draining lymph nodes were pooled and enriched for T cells using a Miltenyi T-cell purification kit. Isolated T cells were counted and co-cultured for 8 hours with dendritic cells loaded with B16F10 tumor cell lysates in the presence of 20 U/ml IL-2 (R and D) plus Brefeldin A (BD Bioscience). After restimulation, lymphocytes were processed for flow cytometry as above.

### 6.1.1.11 Statistics

Data were analyzed by 2-tailed Student's t test, and P<0.05 was considered statistically significant.

### 6.1.2 Results

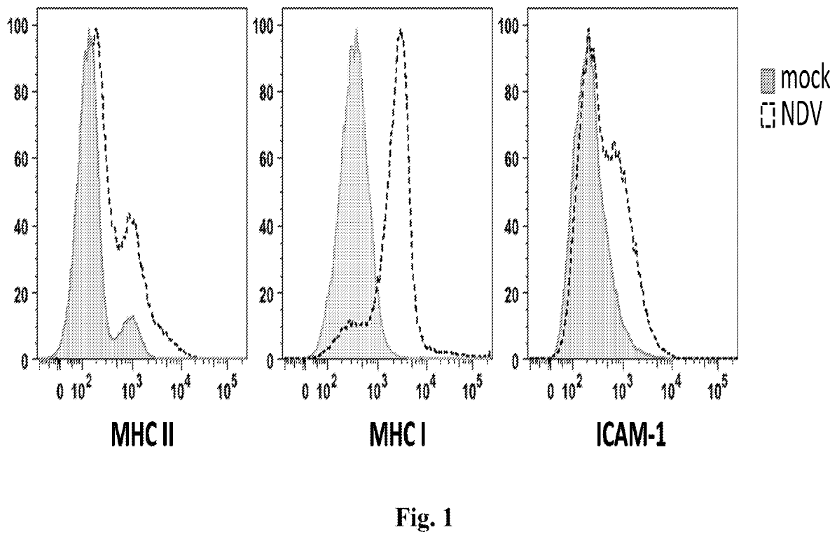

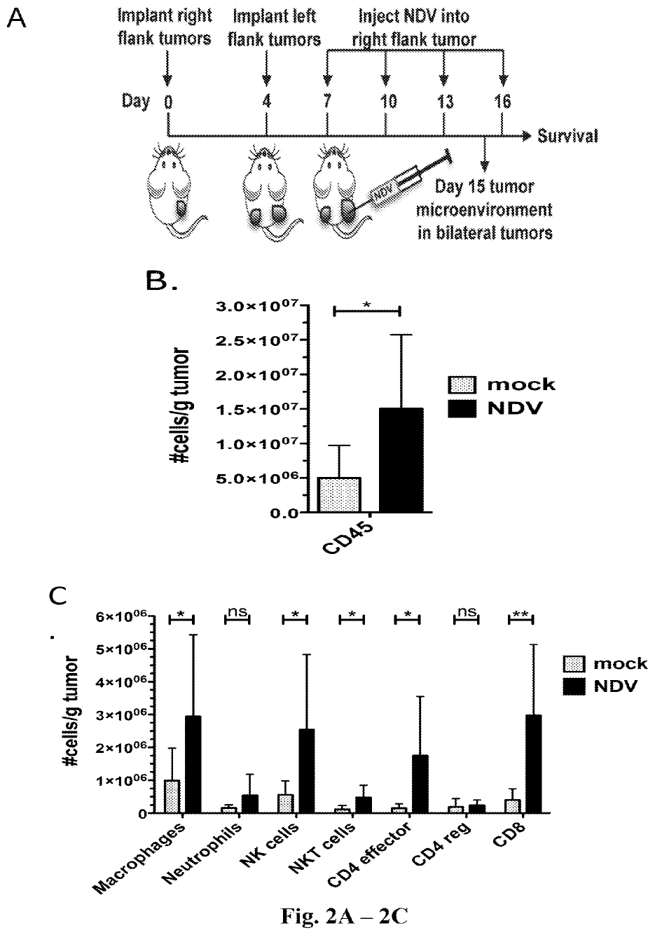

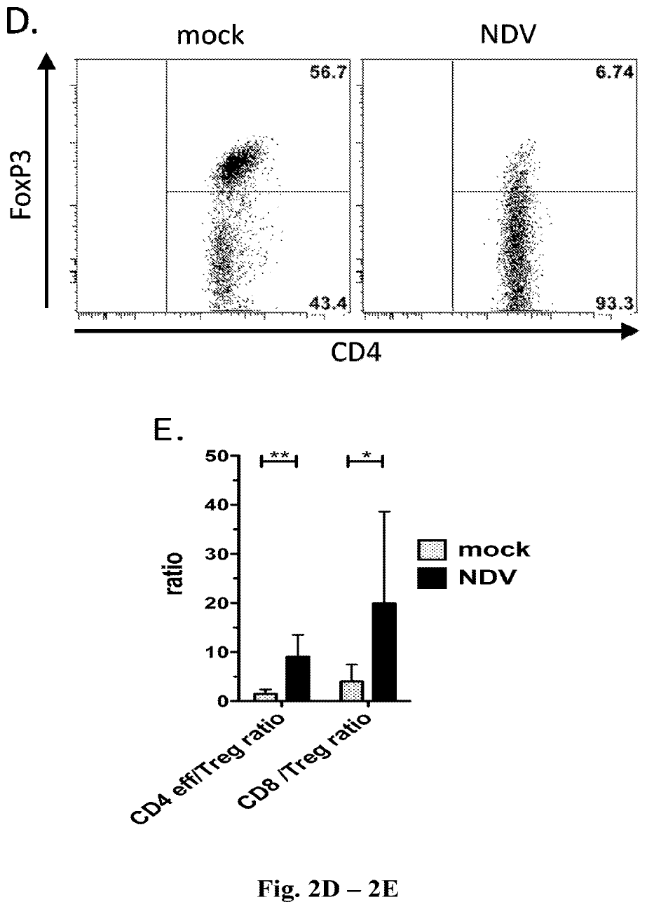

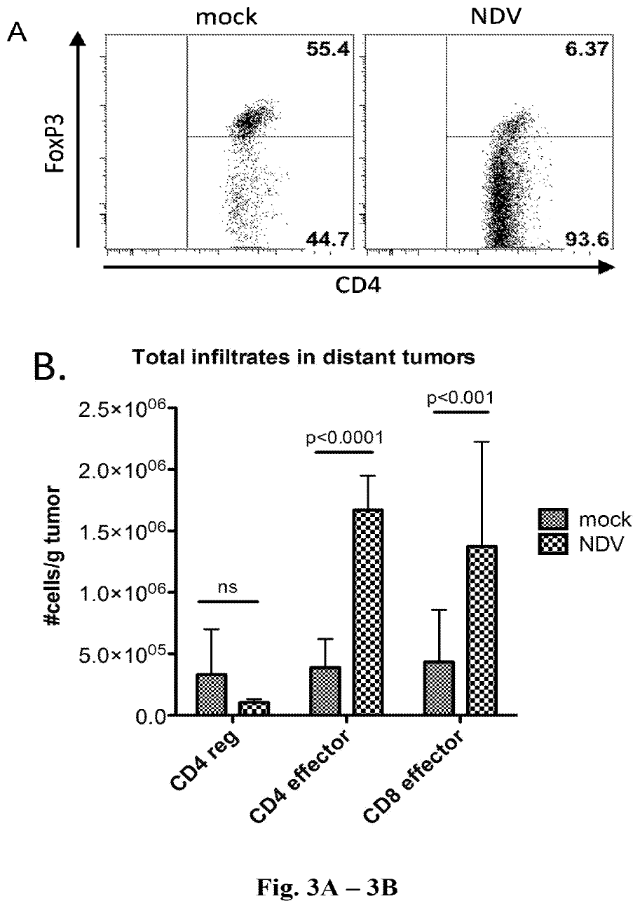

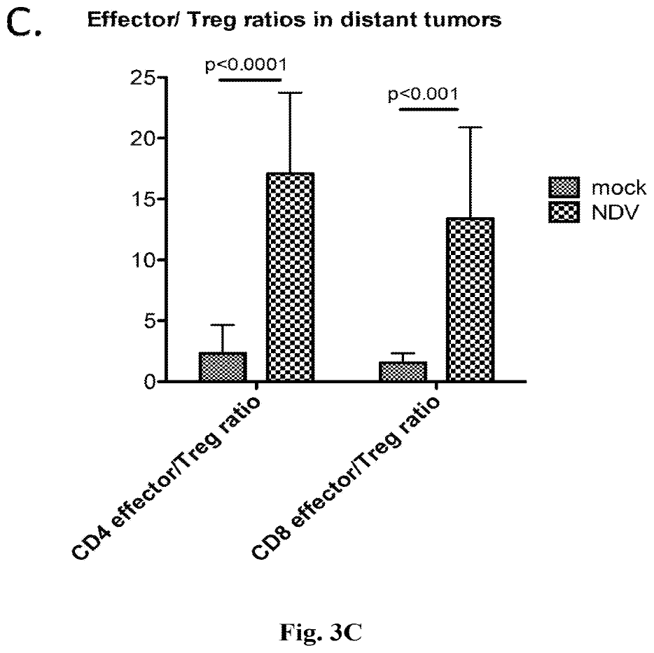

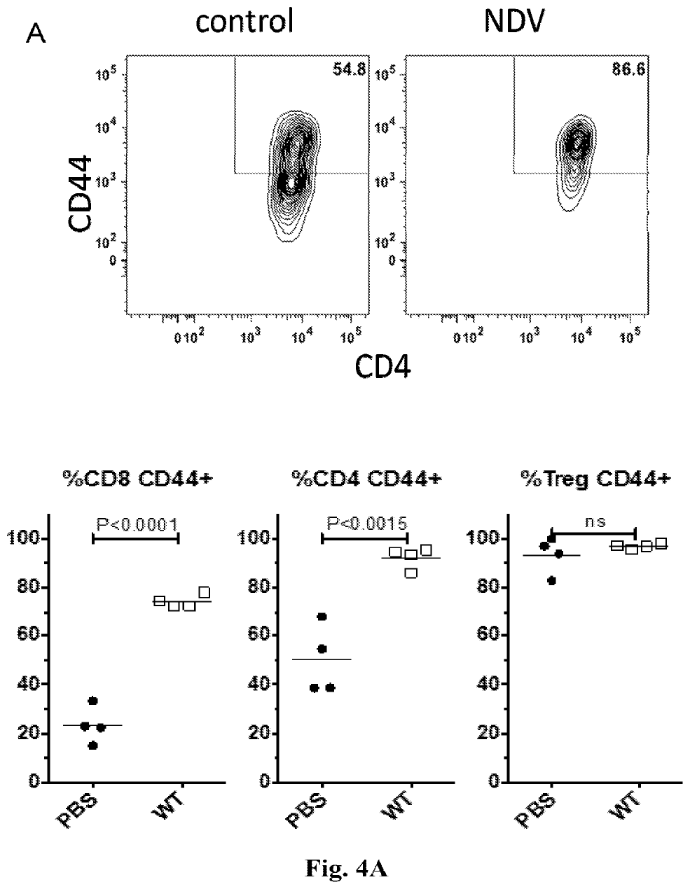

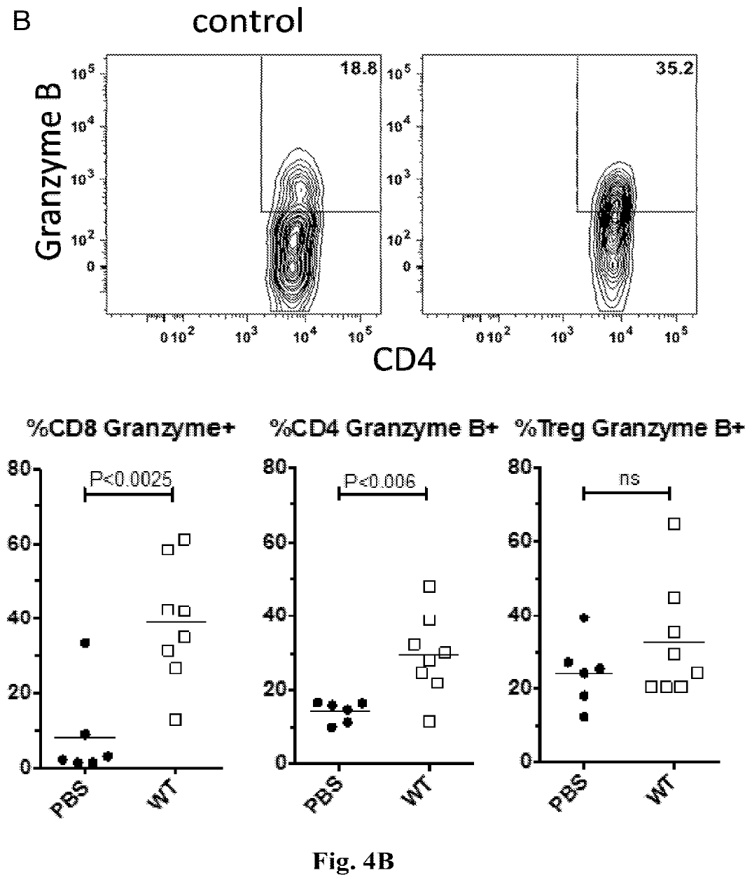

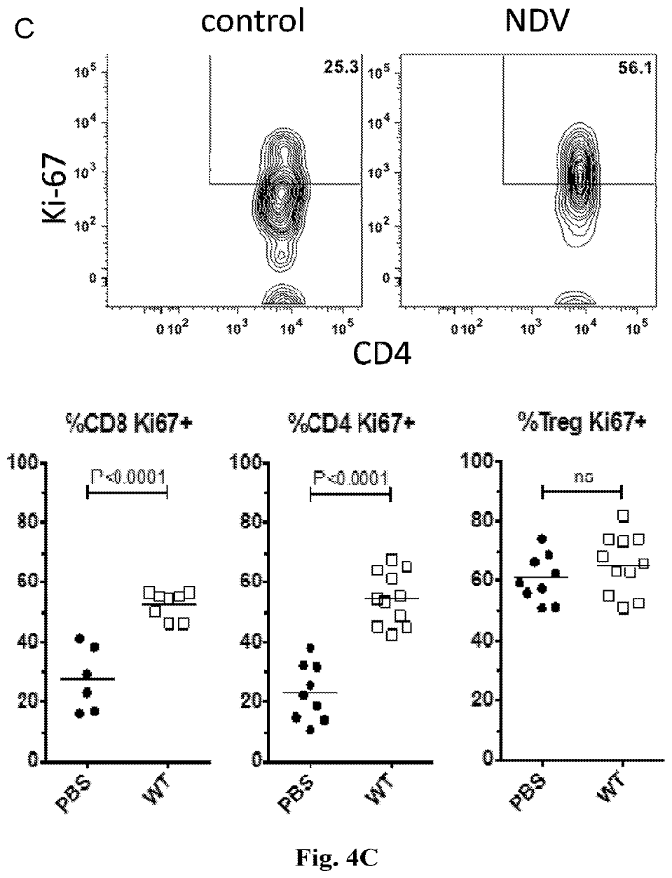

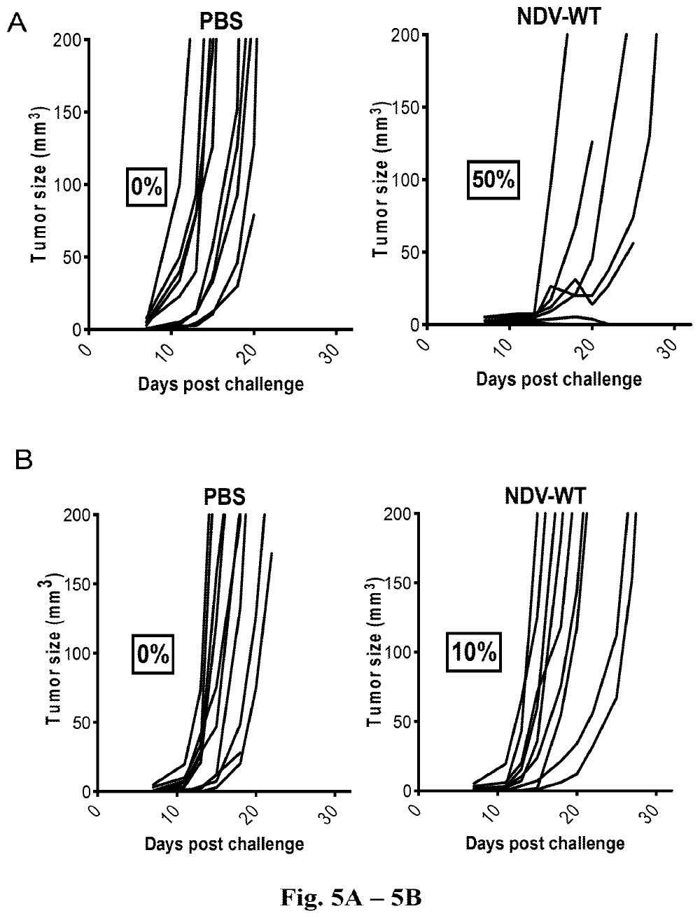

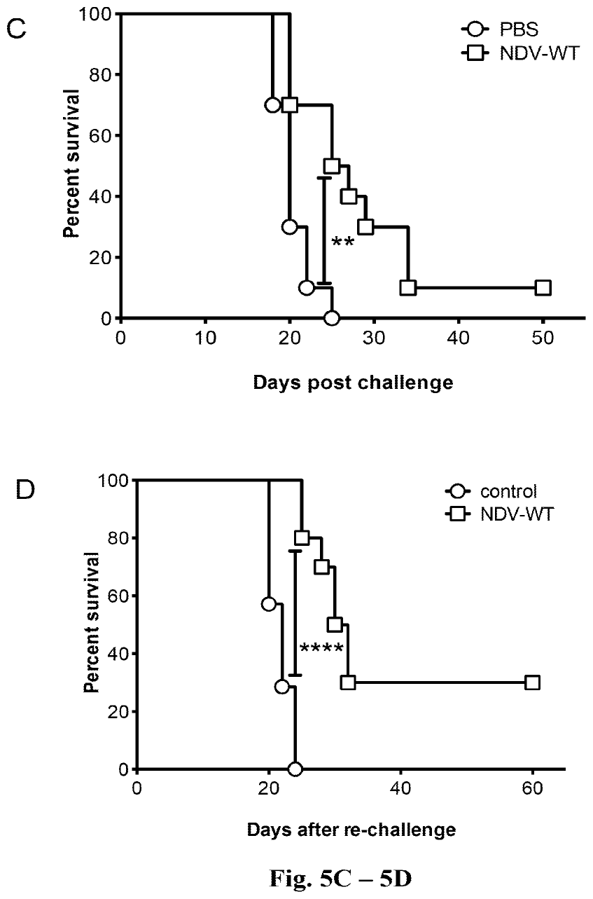

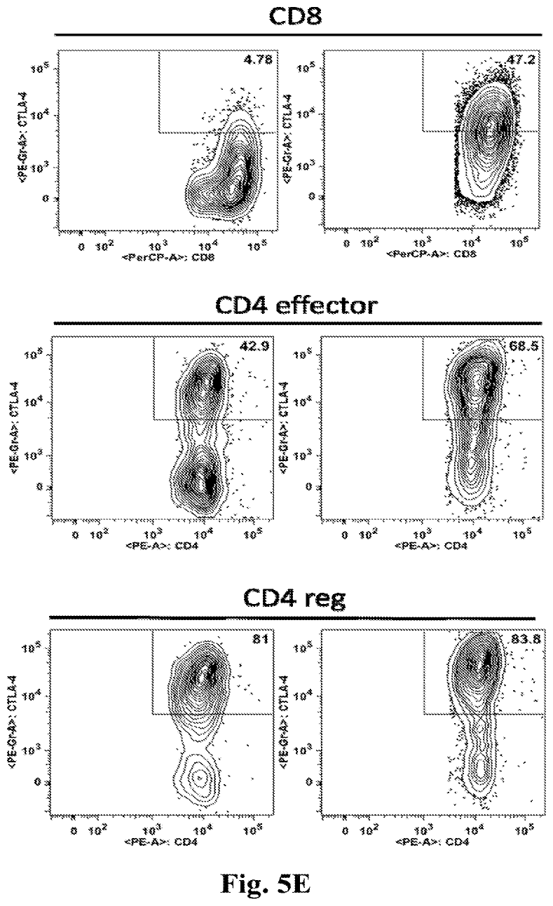

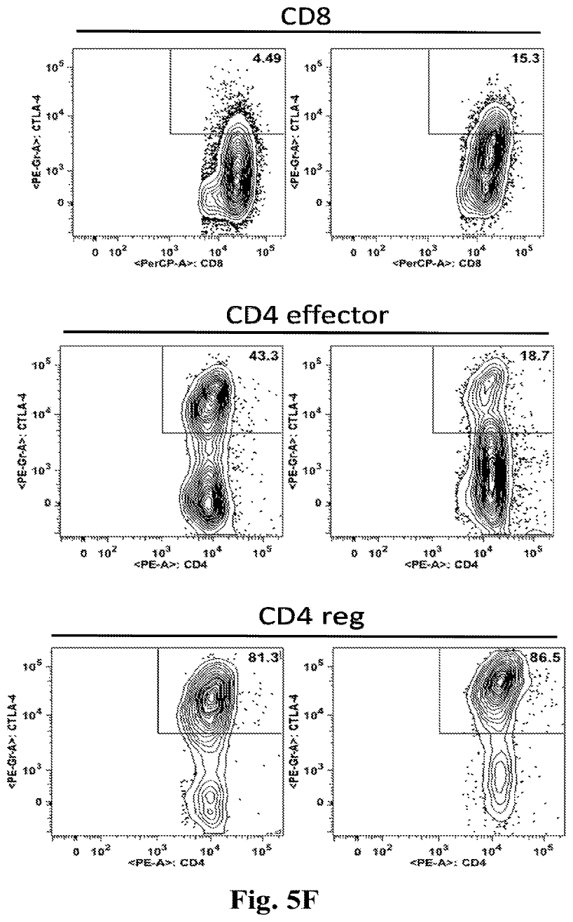

In order to characterize the anti-tumor immune response induced by Newcastle disease virus (NDV) infection, the expression of MHC I and MHC II molecules as well as ICAM-1 on the surface of in vitro infected cells was assessed. As shown in FIG. 1, NDV infection in B16 melanoma cells induces upregulation of MHC class I and II molecules as well as adhesion molecule ICAM-1, all of which are thought to be important for recruitment of tumor-specific lymphocytes and activation of anti-tumor immune response. Next, the anti-tumor immune response induced by NDV infection in vivo was assessed in a murine melanoma model and an established 2-flank model that allowed for monitoring of responses both in the virus-injected tumors as well as distant tumors which do not receive the virus. As shown in FIGS. 2A-2E, the virus-infected tumors show dramatic infiltration with immune cells such as NK cells, macrophages, and CD8 and CD4 cells, but not regulatory T cells. Since part of this immune response could be a response to virus, rather than tumor, the immune response with respect to contralateral tumors was assessed (FIGS. 3A-3C). Interestingly, these tumors demonstrated a similar degree of increased CD8 and CD4 effector, but not T reg infiltrate. Analysis of these cells revealed that they upregulate activation, proliferation, and lytic markers (FIGS. 4A-4C). NDV monotherapy was effective in controlling the treated tumors (FIG. 5A), but only marginally slowed down the growth of the contralateral tumors (FIG. 5B). Mice that cleared the tumors, however, demonstrated some degree of protection against further tumor challenge (FIGS. 5C-5D), suggesting that NDV therapy can induce a lasting immunity.

Next, it was assessed whether additional mechanisms could be targeted to enhance the anti-tumor effect generated by NDV. Characterization of tumor-infiltrating lymphocytes from both NDV-injected and non-injected tumors revealed upregulation of the inhibitory receptor CTLA-4 on lymphocytes (FIG. 5E-F).

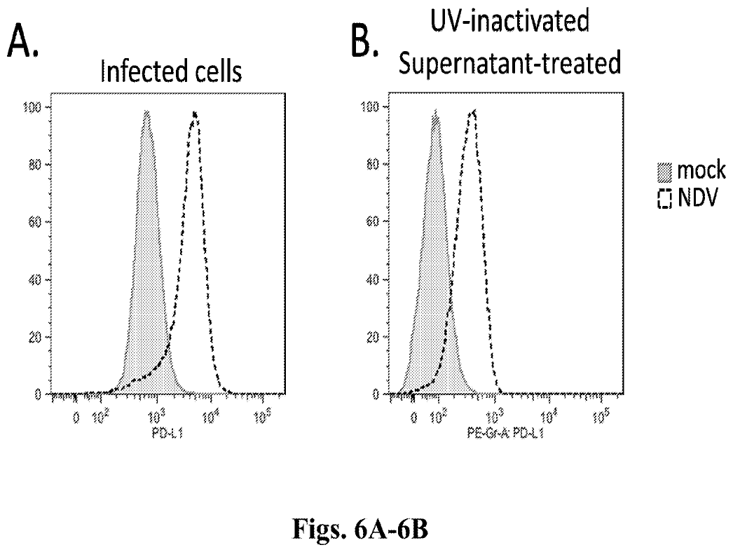

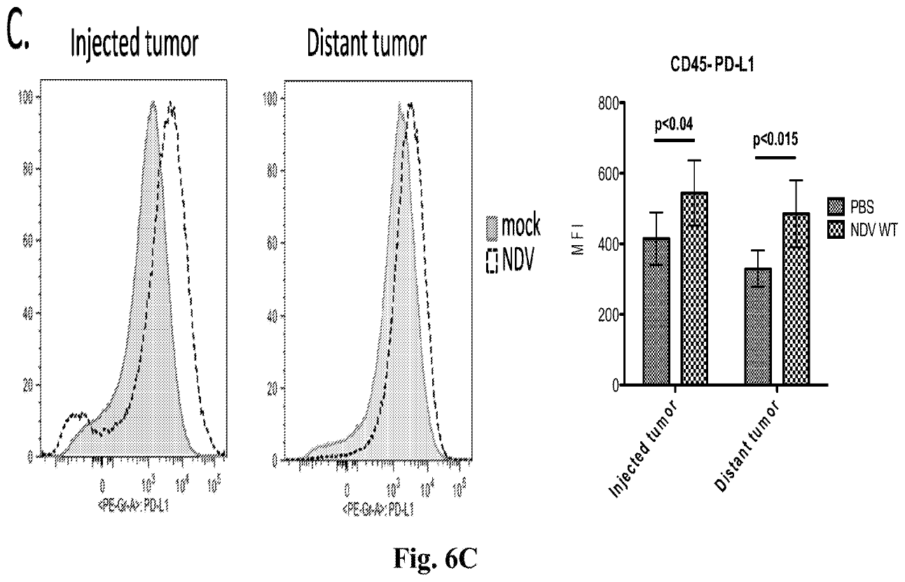

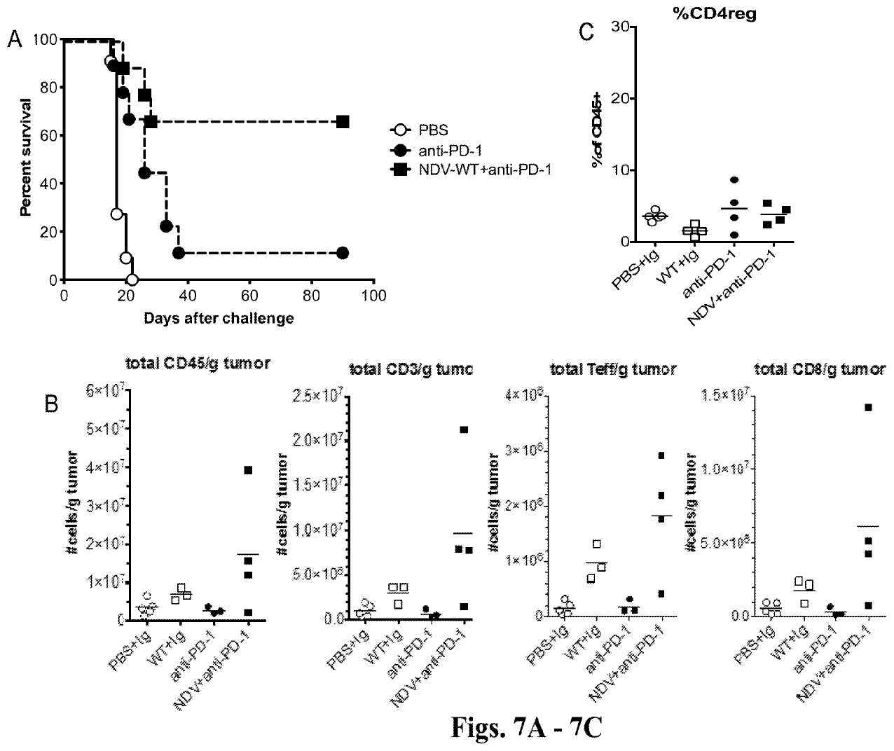

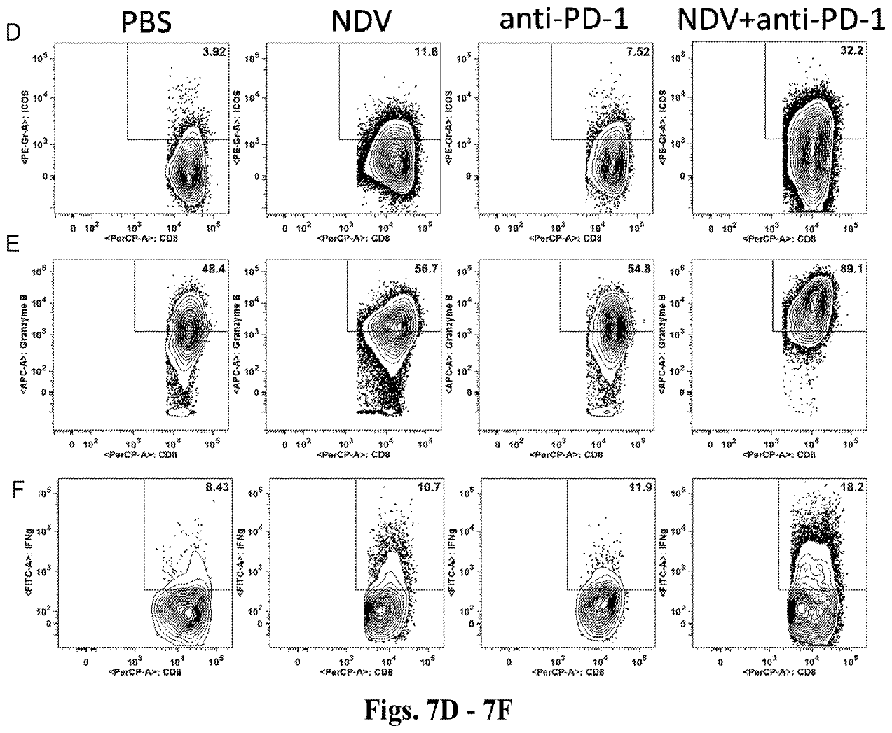

To determine whether targeting immune checkpoints in combination with NDV therapy could be beneficial, the effect on the PD-1-PD-L1 pathway following NDV infection was assessed. As shown in FIGS. 6A-6C, NDV infected tumor cells both in vitro and in vivo had upregulated the expression of the inhibitory PD-L1 ligand on the surface of the cells. This effect was not just a result of a direct virus infection, but was also seen when non-infected cells were treated with UV-inactivated supernatants from the virus infected cells (FIG. 6B) and in contralateral, noninfected, tumors (FIG. 6C). This prompted testing combination therapy with NDV and anti-PD-1 antibody. NDV therapy in combination with anti-PD-1 in the aggressive B16 melanoma model resulted in cures in the majority of animals, an effect that was associated with increased tumor infiltration with activated effector lymphocytes (FIGS. 7A-7F).

Throughout the studies conducted, the therapeutic efficacy of a combination therapy decreased when larger tumor challenge was used. Next, activation markers that could predict a better response and could be targeted for further improvement in therapeutic efficacy were assessed. ICOS upregulation has been previously been shown to be associated with more durable therapeutic responses and increased survival in patients treated with anti-CTLA-4 therapy for malignant melanoma.

Overall, these studies demonstrate that combination of NDV with immune checkpoint regulatory antibodies can be used as a strategy to circumvent the limitation of both oncolytic virus therapy and antibody therapy. This finding has clinical application.

### 6.2 Example 2

This example demonstrates the anti-tumor immune responses induced by oncolytic NDV and the anti-tumor responses induced by NDV in combination with PD-1 blockade.

### 6.2.1 Materials & Methods

### 6.2.1.1 Mice

C57BL/6J and Balb/C mice were purchased from Jackson Laboratory. IFNAR−/− mice on C57BL/6J background were a kind gift of Dr. Eric Pamer. Pmel-1 and Trp=1 TCR transgenic mice have been reported (Overwijk et al., 2003, J. Exp. Med, 198:568, Muransky et al., 2008, Blood 112:362) and were provided by N. Restifo (National Cancer Institute, Bethesda, MD). Trp1 mice were crossed to CD2: luciferase mice provided by Patrick Hwu at MD Anderson Cancer Center (Houston, TX) to create Trp1 Luciferase+ (Trp1-Fluc) mice. All mice were maintained in microisolator cages and treated in accordance with the NIH and American Association of Laboratory Animal Care regulations. All mouse procedures and experiments for this study were approved by the Memorial Sloan-Kettering Cancer Center Institutional Animal Care and Use Committee.

### 6.2.1.2 Cell Lines

The murine cancer cell lines for melanoma (B16F10), and colon carcinoma (CT26 and MC38) were maintained in RPMI medium supplemented with 10% fetal calf serum and penicillin with streptomycin. The murine prostate cancer cell line TRAMP-C2 was maintained in DMEM medium supplemented with 5% fetal calf serum (FCS; Mediatech, Inc.), 5% Nu Serum IV (BD Biosciences) HEPES, 2-ME, pen/strep, L-glut, 5 μg/mL insulin (Sigma), and 10 nmol/L DHT (Sigma).

### 6.2.1.3 Antibodies

Therapeutic anti-PD-1 (clone RMP1-14), anti-PD-L1 (clone 9G2), anti-CD8 (clone 2.43), anti-CD4 (clone GK1.5), anti-IFN-gamma (clone XMG1.2), and anti-NK1.1 (clone PK136) monoclonal antibodies were produced by BioXcell. Antibodies used for flow cytometry were purchased from eBioscience, Biolegend, Invitrogen, and BD Pharmingen.

### 6.2.1.4 Viruses and Cloning

Recombinant lentogenic NDV LaSota strain was used for all experiments. Viruses were rescued from cDNA using methods described previously and sequenced by reverse transcription PCR for insert fidelity. Virus titers were determined by serial dilution and immunofluorescence in Vero cells.

### 6.2.1.5 In Vitro Infection Experiments

For cell surface labeling, cells were infected in 6-well dishes at MOI 2 (B16F10) or MOI 5 (TRAMP C2) in triplicate. Twenty-four hours later, the cells were harvested by scraping and processed for surface labeling and quantification by flow cytometry. For in vitro cytotoxicity experiments, cells were infected at the indicated MOI's and incubated at 37° C. in serum-free media in presence of 250 ng/ml TPCK trypsin. At 24, 48, 72, and 96 hours post infection the cells were washed and incubated with 1% Triton X-100 at 37° C. for 30 minutes. LDH activity in the lysates was determined using the Promega CytoTox 96 assay kit, according to the manufacturer's instructions.

### 6.2.1.6 Tumor Challenge Survival Experiments

Bilateral flank tumor models were established to monitor for therapeutic efficacy in both injected and systemic tumors. Treatment schedules and cell doses were established for each tumor model to achieve 10-20% tumor clearance by NDV. For experiments evaluating combination therapy of NDV with anti-PD-1 antibody, B16F10 tumors were implanted by injection of 2×10⁵ B16F10F10 cells in the right flank intradermally (i.d.) on day 0 and 5×10⁴ cells in the left flank on day 4. On days 7, 9, 11, and 13 the mice were treated with intratumoral injections of 2×10⁷ pfu of NDV in PBS in a total volume of 100 μl. Concurrently, on days 7, 9, 11, and 13 the mice received intraperitoneal (i.p.) injections of anti-PD-1 antibody (250 μg) or anti-PD-L1 antibody (250 μg). Control groups received a corresponding dose of isotype antibody i.p. and intratumoral injection of PBS. The animals were euthanized for signs of distress or when the total tumor volume reached 1000 mm³. For depletion of immune cells, mice were injected i.p. with 500 μg of monoclonal antibodies to CD8+, CD4+, NK1.1 or IFNγ one day before and two days after tumor challenge, followed by injection of 250 μg every 5 days throughout the experiment. For the TRAMP-C2 model, 1×10⁶ cells were implanted in the right flank on day 0 and 5×10⁵ cells were implanted in the left flank on day 4. Treatment was performed on days 7, 10, 13, and 16 in the similar fashion to above. For the CT26 model, tumors were implanted by injection of 1×10⁶ CT26 cells in the right flank i.d. on day 0 and 1×10⁶ cells in the left flank on day 2. Treatment was carried out as above on days 6, 9, and 12. Isolation of Trp1 and Pmel lymphocytes and adoptive transfer

Spleens and lymph nodes from transgenic mice were isolated and grinded through 70-um nylon filters. CD4+ and CD8+ cells were purified by positive selection using Miltenyi magnetic beads.

The isolated Trp1 or Pmel cells were injected into recipient animals via the tail vein at the indicated schedule at 2.5×10⁴ cells per mouse and 1×10⁶ cells per mouse, respectively.

### 6.2.1.7 Serum Transfer Experiments

Groups of tumor-bearing mice were treated intratumorally with single injection of NDV or PBS. On day 4, blood was collected by terminal bleeding and serum was isolated by centrifugation. Sera were pooled from each group and UV-treated in Stratalinker 1800 with six pulses of 300mJ/cm² UV light to inactivate any virus that could be potentially present. Undiluted 100 μl of serum was injected intratumorally into naïve B16F10 tumor-bearing mice for a total of 3 injections given every other day. Tumors were removed 3 days after the last injection and processed for isolation of tumor-infiltrating lymphocytes as described below.

### 6.2.1.8 Bioluminescence Imaging

Mice were imaged every 2-3 days starting on day 6. Mice were injected retro-orbitally with 50 μl of 40 mg/ml D-luciferin (Caliper Life Sciences) in PBS and imaged immediately using the IVIS Imaging System (Caliper Life Sciences). Gray-scale photographic images and bioluminescence color images were superimposed using The Living Image, version 4.0 (Caliper Life Sciences) software overlay. A region of interest (ROI) was manually selected over the tumor and the area of the ROI was kept constant.

### 6.2.1.9 Isolation of Tumor-Infiltrating Lymphocytes

B16F10 tumors were implanted by injection of 2×10⁵ B16F10 cells in the right flank i.d. on day 0 and 2×10″ cells in the left flank on day 4. On days 7, 9, and 11 the mice were treated with intratumoral injections of 2×10⁷ pfu of NDV, and i.p. anti-PD-1 antibody where specified. Rare animals that died from tumor burden (always in untreated control groups) or animals that completely cleared the tumors (always in treatment groups) were not used for the analysis. On day 15, mice were sacrificed and tumors and tumor-draining lymph nodes were removed using forceps and surgical scissors and weighed. Tumors from each group were minced with scissors prior to incubation with 1.67 Wünsch U/mL Liberase and 0.2 mg/mL DNase for 30 minutes at 37° C. Tumors were homogenized by repeated pipetting and filtered through a 70-μm nylon filter. Cell suspensions were washed once with complete RPMI and purified on a Ficoll gradient to eliminate dead cells. Cells from tumor draining lymph nodes were isolated by grinding the lymph nodes through a 70-μm nylon filter.

### 6.2.1.10 Flow Cytometry

Cells isolated from tumors or tumor-draining lymph nodes were processed for surface labeling with several antibody panels staining for CD45, CD3, CD4, CD8, CD44, ICOS, CD11c, CD19, NK1.1, CD11b, F4/80, Ly6C and Ly6G. Fixable viability dye eFluor506 (eBioscience) was used to distinguish the live cells. Cells were further permeabilized using FoxP3 fixation and permeabilization kit (eBioscience) and stained for Ki-67, FoxP3, Granzyme B, CTLA-4, and IFNγ. Data was acquired using the LSRII Flow cytometer (BD Biosciences) and analyzed using FlowJo software (Treestar).

### 6.2.1.11 DC Purification and Loading

Spleens from naïve mice were isolated and digested with 1.67 Wünsch U/mL Liberase and 0.2 mg/mL DNase for 30 minutes at 37° C. The resulting cell suspensions were filtered through 70 μm nylon filter and washed once with complete RPMI. CD11c+DC's were purified by positive selection using Miltenyi magnetic beads. Isolated DC's were cultured overnight with recombinant GM-CSF and B16F10 tumor lysates and were purified on Ficoll gradient.

### 6.2.1.12 Analysis of Cytokine Production

Cell suspensions from tumors or tumor-draining lymph nodes were pooled and enriched for T cells using a Miltenyi T-cell purification kit. Isolated T cells were counted and co-cultured for 8 hours with DC's loaded with B16F10 tumor cell lysates in the presence of 20 U/ml IL-2 (R and D) plus Brefeldin A (BD Bioscience). After restimulation, lymphocytes were processed for flow cytometry as above.

### 6.2.1.13 Immunofluorescence and Microscopy

Tumors were dissected from the mice, washed in PBS, fixed in 4% paraformaldehyde, and processed for paraffin embedding according to protocols described previously. Sections were cut using a microtome, mounted on slides, and processed for staining with hematoxylin and cosin (H&E) or with anti-CD3 and anti-FoxP3 antibody. Slides were analyzed on Zeiss Axio 2 wide-field microscope using 10× and 20× objectives.

### 6.2.1.14 Statistics

Data were analyzed by 2-tailed Student's t test (for comparisons of 2 groups) and ANOVA where appropriate. Data for survival were analyzed by Log-Rank (Mantel-Cox) Test. Two-sided p<0.05 was considered statistically significant (P≤0.05 (*), P≤0.01 (**), P<0.001 (***), P<0.0001 (****)).

### 6.2.2 RESULTS

### 6.2.2.1 NDV Replication is Restricted to the Injected Tumor Site

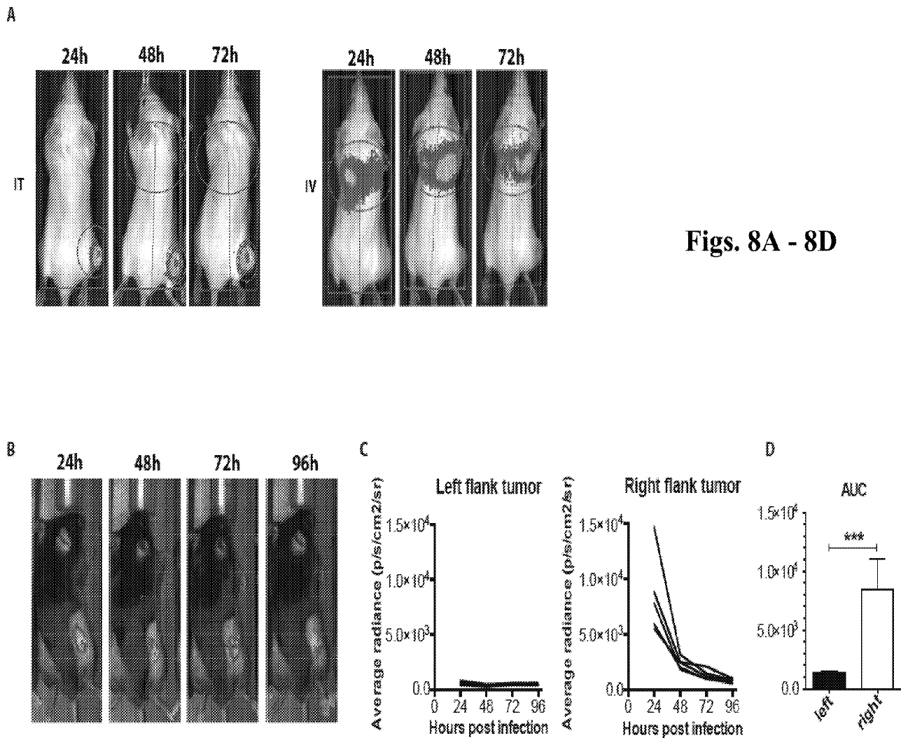

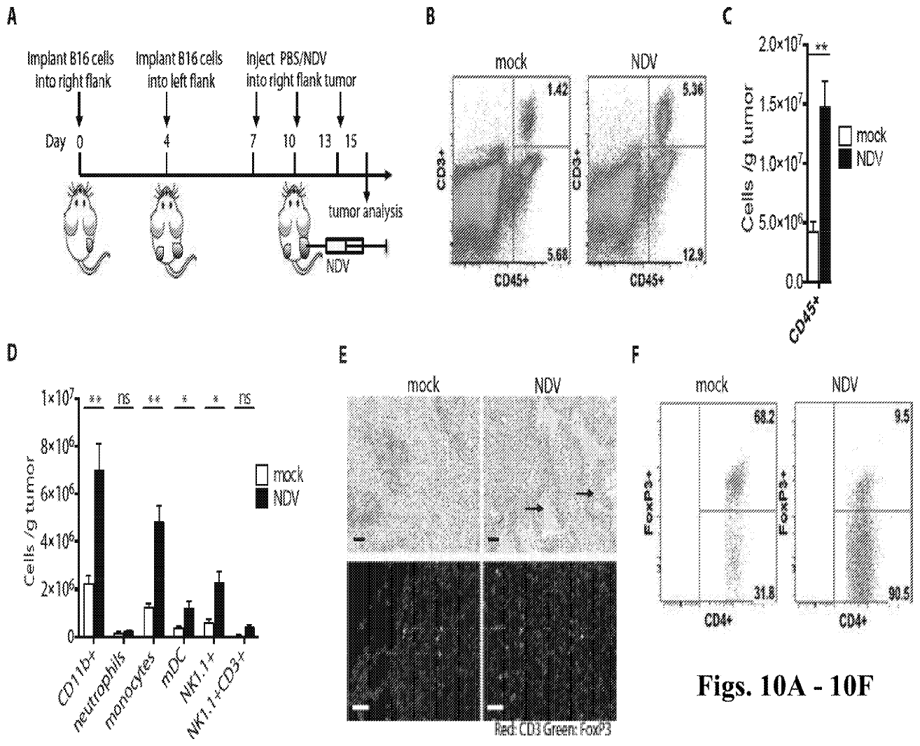

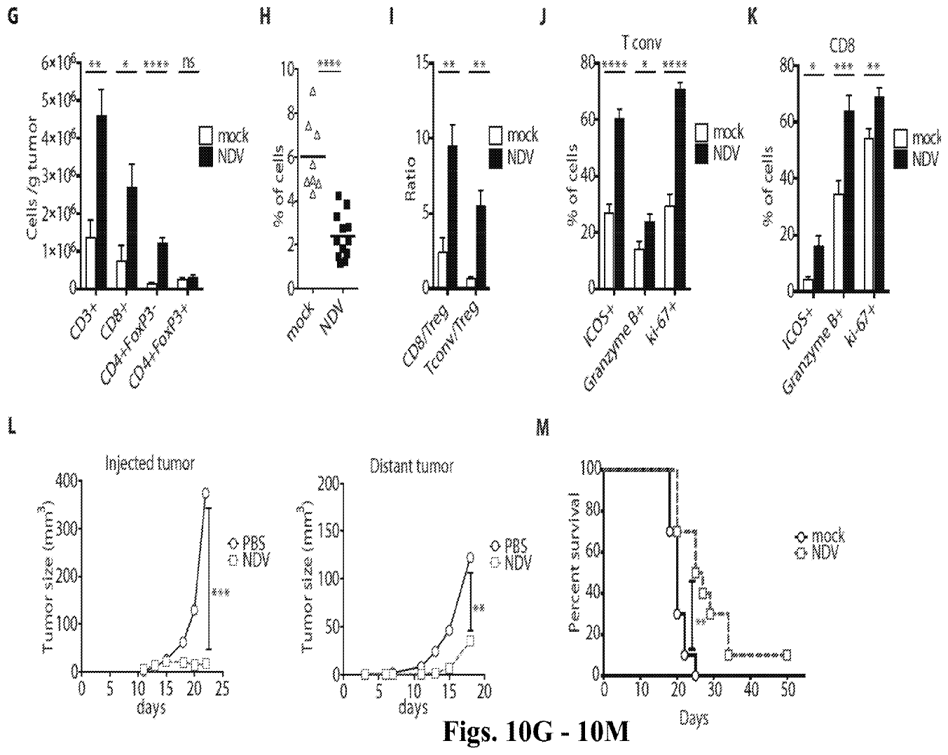

The viral distribution kinetics with intratumoral and systemic administration of NDV were characterized. Intratumoral injection of recombinant NDV expressing firefly luciferase reporter (NDV-Fluc) resulted in sustained luciferase signal in the injected flank tumor, while systemic administration of the virus resulted in no detectable luciferase signal in the tumor (FIG. 8A). As limited systemic virus delivery was unlikely to induce sufficient tumor lysis and immune response, the intratumoral NDV injection was explored as a means to elicit an anti-tumor immune response that could potentially overcome the limitations of systemic OV therapy. As such, for further studies modeled metastatic disease was modeled by using the bilateral flank B16F10 tumor model (FIG. 10A). NDV-Fluc administration into the right flank tumor resulted in viral replication within the injected tumor, with the luciferase signal detectable for up to 96 hours (FIGS. 8B-8D). No virus was detected in the contralateral (left flank) tumor by luminescent imaging (FIGS. 8B-8D), by passage in embryonated eggs, or RT-PCR. This system thus allowed for the characterization of the immune responses in both virus-injected and distant tumors, which were not directly affected by NDV.

### 6.2.2.2 NDV Therapy Increases Local and Distant Tumor Lymphocyte Infiltration and Delays Tumor Growth

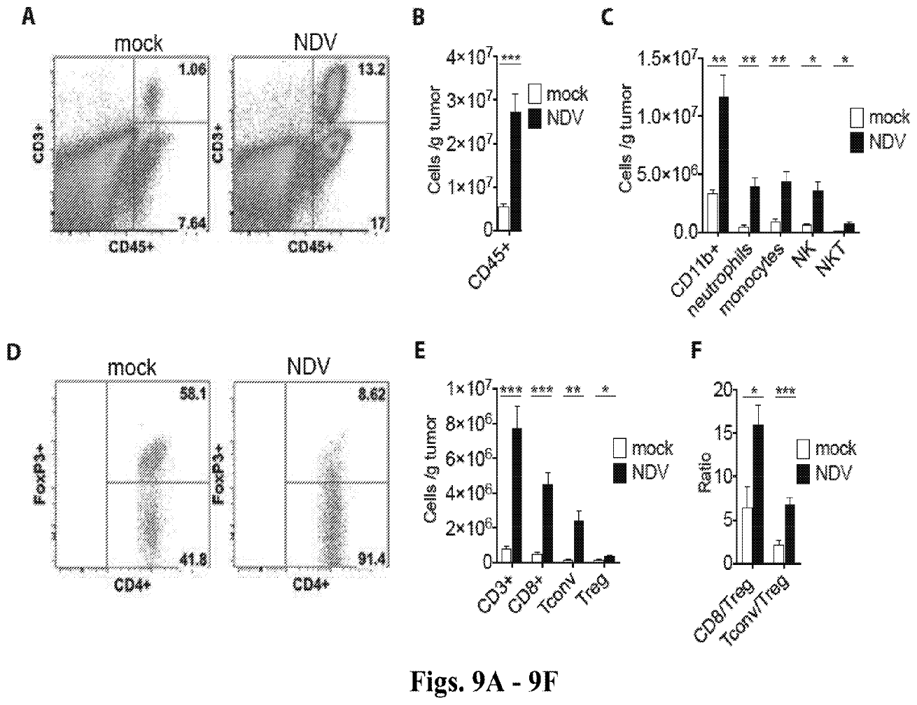

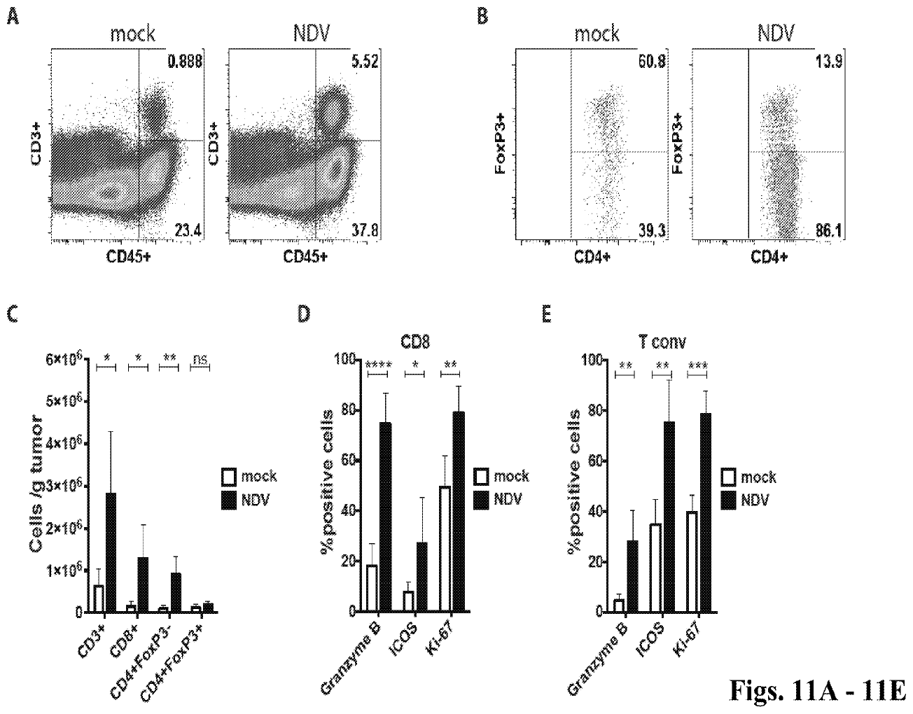

Analysis of the virus-injected tumors revealed an inflammatory response as evidenced by increased infiltration with cells expressing leukocyte common antigen CD45 (FIGS. 9A-9B). The immune infiltrates were characterized by increase in innate immune compartment, including myeloid cells, NK cells, and NKT cells (FIG. 9C), and the adaptive compartment, including CD8+ and conventional CD4+FoxP3−(Tconv) T cells, leading to significant increase of CD8 and Tconv to regulatory (Treg) T cell ratios (p=0.0131 and p=0.0006, respectively) (FIGS. 9D-9F). Remarkably, analysis of the contralateral tumors revealed a similar increase in the inflammatory infiltrates (FIGS. 10B and 10C), characterized by increased numbers of both innate immune cells (FIG. 10D)) and effector T cells (FIGS. 10E and 10G). Notably, although there were no major changes in the absolute number of Tregs (FIG. 10G), there was a substantial decrease in their relative percentages (FIGS. 10E, 10F, and 10H), with significant enhancement of the CD8 and Tconv to Treg ratios (p=0.002 and p=0.0021, respectively) (FIG. 10I). Effector T cells isolated from the distal tumors expressed increased activation, proliferation, and lytic markers ICOS, Ki-67, and Granzyme B, respectively (FIGS. 10J and 10K). As previously, virus or viral RNA was unable to be isolated from the distant tumors, suggesting that the observed changes in the distant tumor microenvironment were not due to direct viral infection. In order to further exclude the possibility of undetectable local viral spread, tumors were implanted at other distant sites, such as bilateral posterior footpads, which generated similar findings (FIGS. 11A-11E).

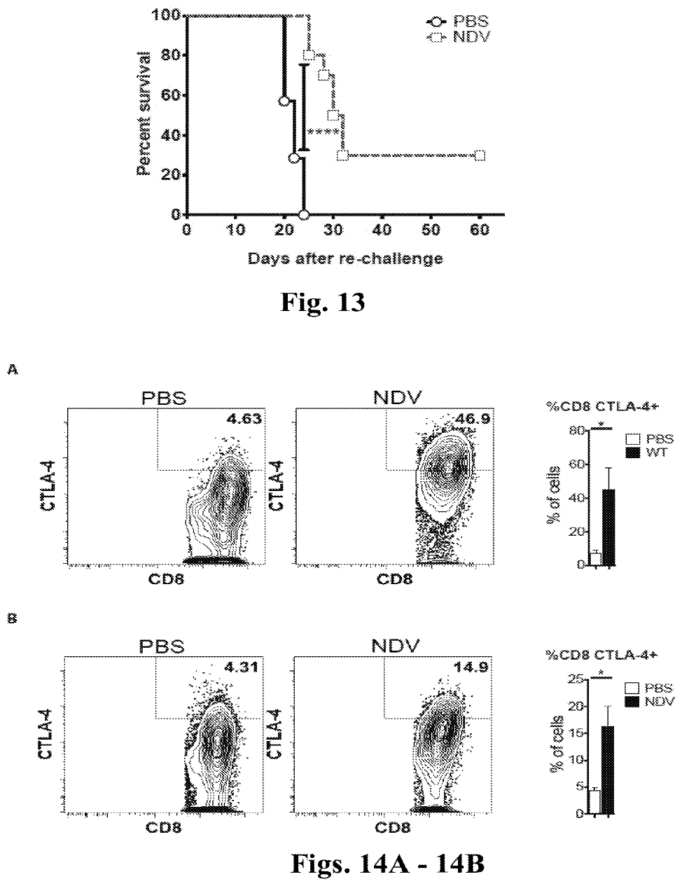

Consistent with the observed inflammatory effect, intratumoral administration of NDV resulted in growth delay not only of the injected, but also of the contralateral tumors, resulting in prolonged animal survival (FIGS. 10L and 10M). To determine whether this effect was transient and whether durable anti-tumor protection was possible, single-flank B16F10 tumor-bearing mice were intratumorally treated with NDV, and long-term survivors were injected with B16F10 cells on the opposite flank. The majority of the animals demonstrated tumor growth delay, and 30% of the animals completely rejected rechallenged cells, suggesting that intratumoral therapy with NDV can indeed induce protective anti-tumor memory responses (FIG. 13).

### 6.2.2.3 NDV Induces Tumor Infiltration and Expansion of Tumor-Specific Lymphocytes

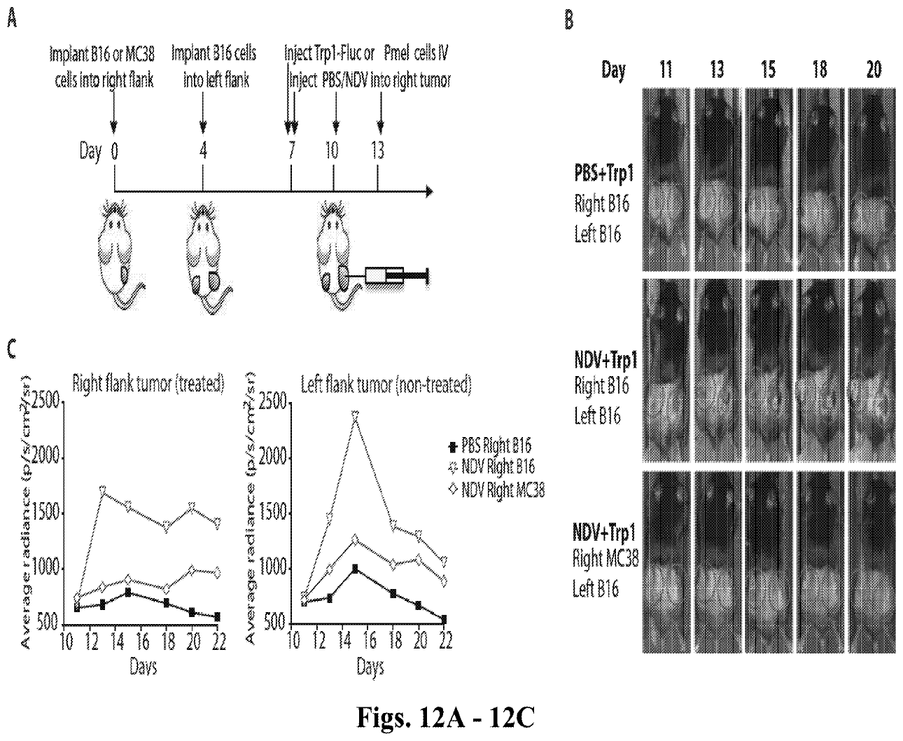

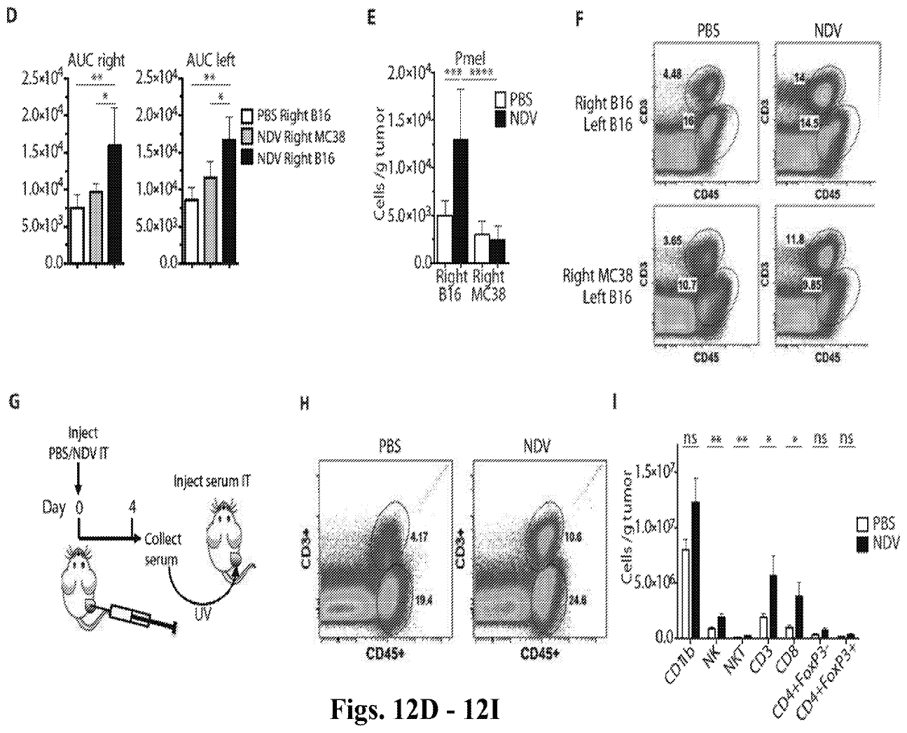

To determine whether the anti-tumor immune response was dependent on the NDV-injected tumor type or a result of nonspecific inflammation generated by NDV infection, the experiment was performed with heterologous tumors (MC38 colon carcinoma and B16F10 melanoma) implanted at the opposite flanks (FIG. 12A). To track tumor-specific lymphocytes, T cell receptor-transgenic congenically-marked CD8+ (Pmel) cells or luciferase-marked CD4+ (Trp1) cells recognizing the melanoma differentiation antigens gp100 (Pmel) and Trp1 (Trp1) were adoptively transferred (Muranski et al., 2008, Blood, 112:362; Overwijk et al., 2003, J Exp Med, 198:569). Bioluminescent imaging was used to measure the distribution and expansion kinetics of the adoptively transferred Trp1 cells. Transfer of Trp1 cells into PBS-treated tumor-bearing animals failed to result in Trp1 accumulation in the tumors, highlighting the highly immunosuppressive nature of the tumor microenvironment in this model (FIGS. 12B-12D). NDV injection into B16F10 tumors resulted in significant increase in the luciferase signal within the injected tumors (FIGS. 12B-12D), indicating Trp1 T cell expansion (area under the curve (AUC) p=0.0084). Remarkably, similar expansion was seen in the contralateral tumor, albeit at a delay (p=0.0009) (FIGS. 12B-12D). In contrast, NDV injection into MC38 tumors failed to induce substantial Trp1 infiltration into the injected MC38 tumors or distant B16F10 tumors (FIGS. 12B-12D), suggesting that the distant tumor-specific lymphocyte infiltration is likely dependent on the antigen identity of the injected tumor. Similarly, intratumoral injection of NDV resulted in increased infiltration of Pmel cells in distant tumors, which was more pronounced when the injected tumor was B16F10 rather than MC38 (FIG. 12E).

Interestingly, although infiltration of distant B16F10 tumors with adoptively-transferred lymphocytes was dependent on the injected tumor identity, distant tumors did demonstrate increased immune infiltration even when the primary injected tumor was MC38 (FIG. 12F), suggesting that a nonspecific inflammatory response component may also play a role. Indeed, serum from NDV-treated animals, treated with UV irradiation to inactivate any potential virus, induced tumor leukocyte infiltration when injected intratumorally into naïve B16F10 tumor-bearing mice (FIGS. 12G and 12H), with the majority of the increase seen in the NK and CD8+compartments (p=0.0089 and p=0.0443, respectively) (FIG. 12I).

### 6.2.2.4 NDV Upregulates CTLA-4 on Tumor-Infiltrating T Cells

Despite the prominent inflammatory response and growth delay seen in distant tumors, complete contralateral tumor rejection with long-term survival was only seen in approximately 10% of animals (FIG. 10M), suggestive of active immunosuppressive mechanisms in the tumor microenvironment. Characterization of NDV-injected and distant tumors revealed upregulation of CTLA-4 on tumor-infiltrating T cells (FIG. 14).

### 6.2.2.5 NDV Therapy Leads to Upregulation of PD-L1 on Tumor Cells and on Tumor Infiltrating Leukocytes

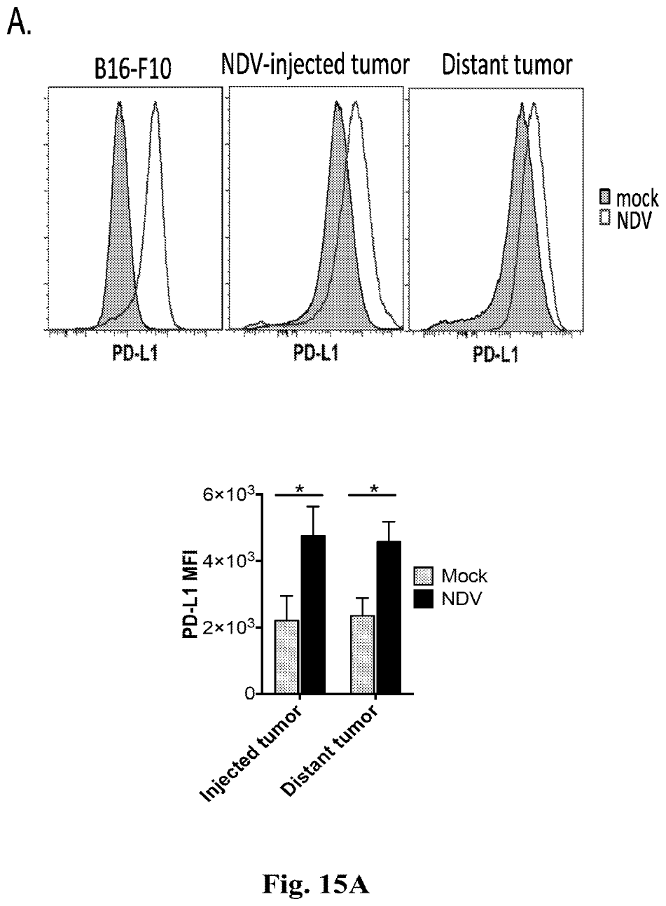

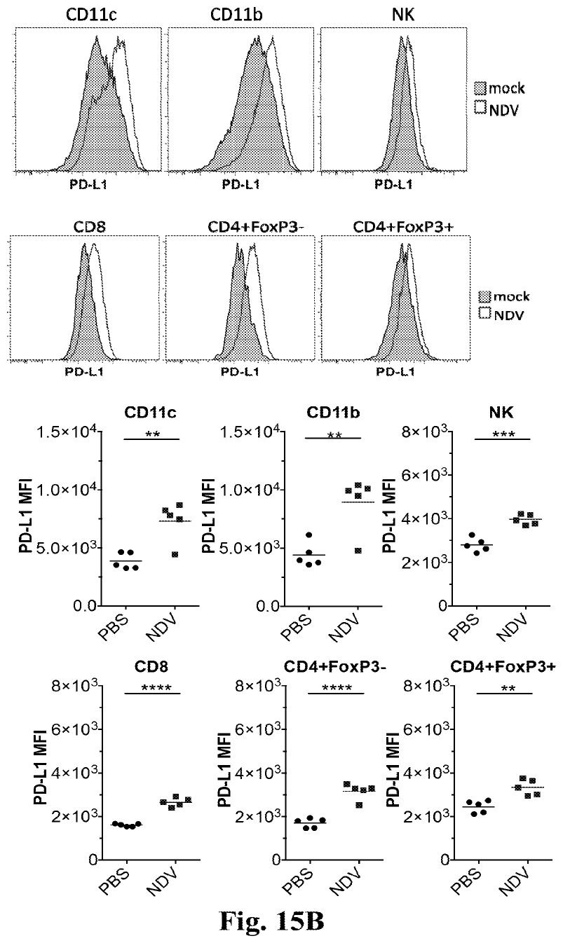

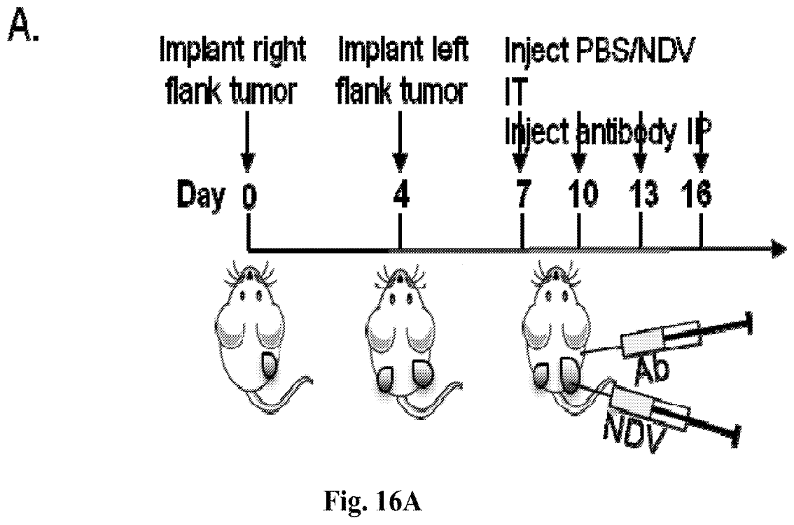

To determine whether targeting other immune checkpoints in combination with NDV therapy could be beneficial, the effect on the PD-1-PD-L1 pathway following NDV infection was assessed. As shown in FIG. 15, NDV infected tumor cells both in vitro and in vivo had upregulated the expression of the inhibitory PD-L1 ligand on the surface of the cells (FIG. 15A), which was also seen in the distant, noninfected, tumors. The upregulation of PD-L1 was not just restricted to tumor cells, but was also seen on tumor infiltrating leukocytes of both innate and adaptive immune lineages (FIG. 15B).

### 6.2.2.6 Combination Therapy of NDV with PD-1 and PD-L1-Blocking Antibodies Leads to Improved Anti-Tumor Immunity and Long-Term Animal Survival

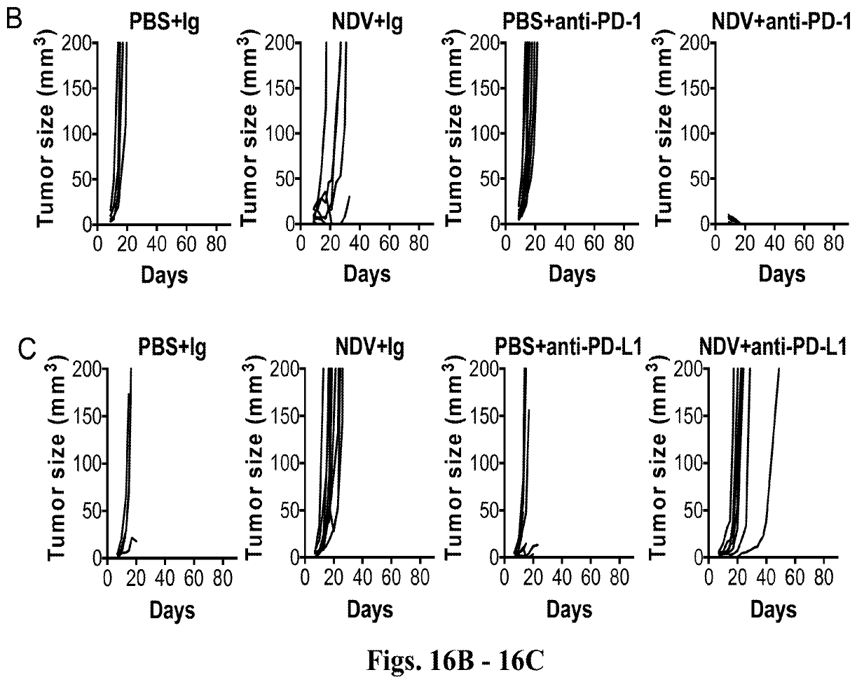

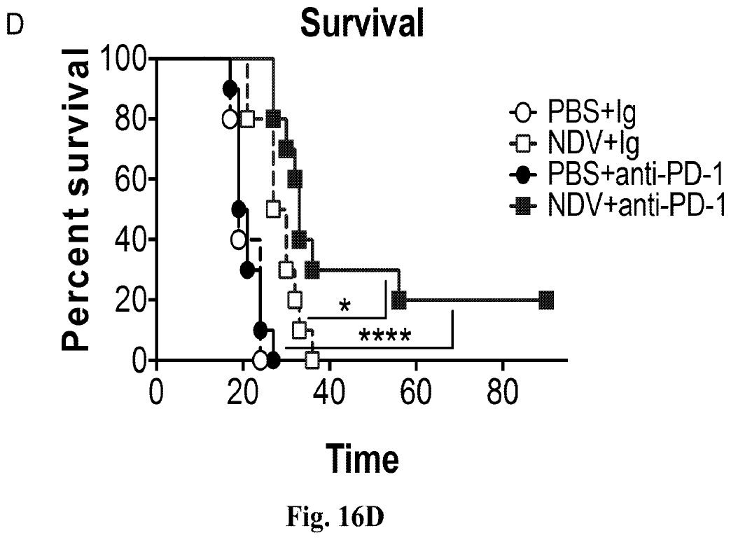

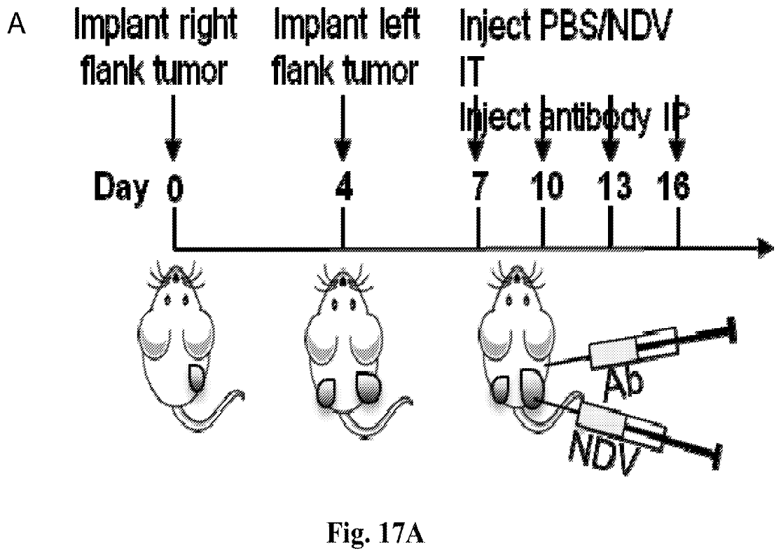

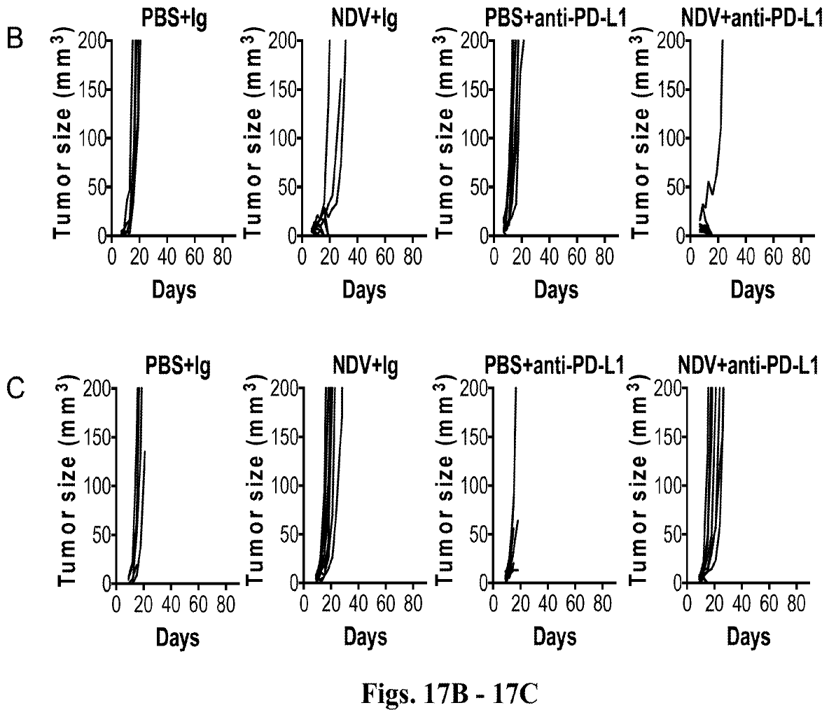

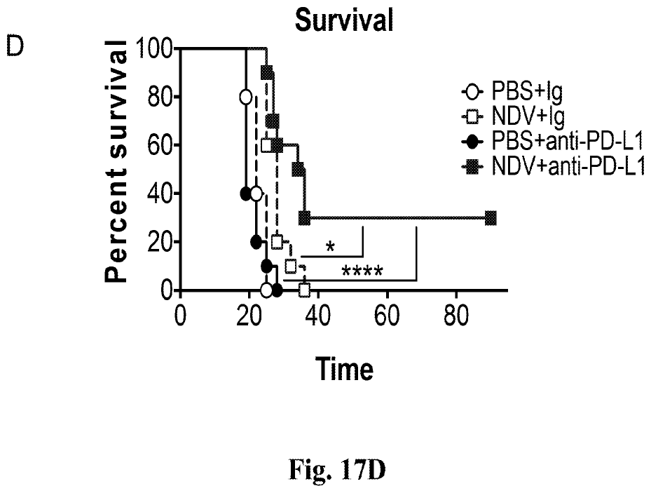

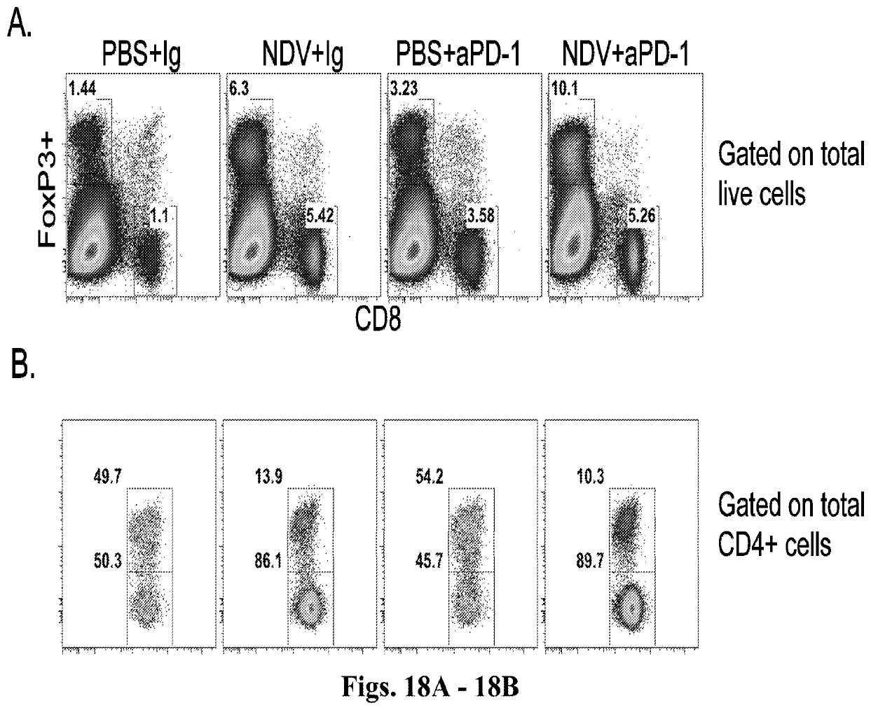

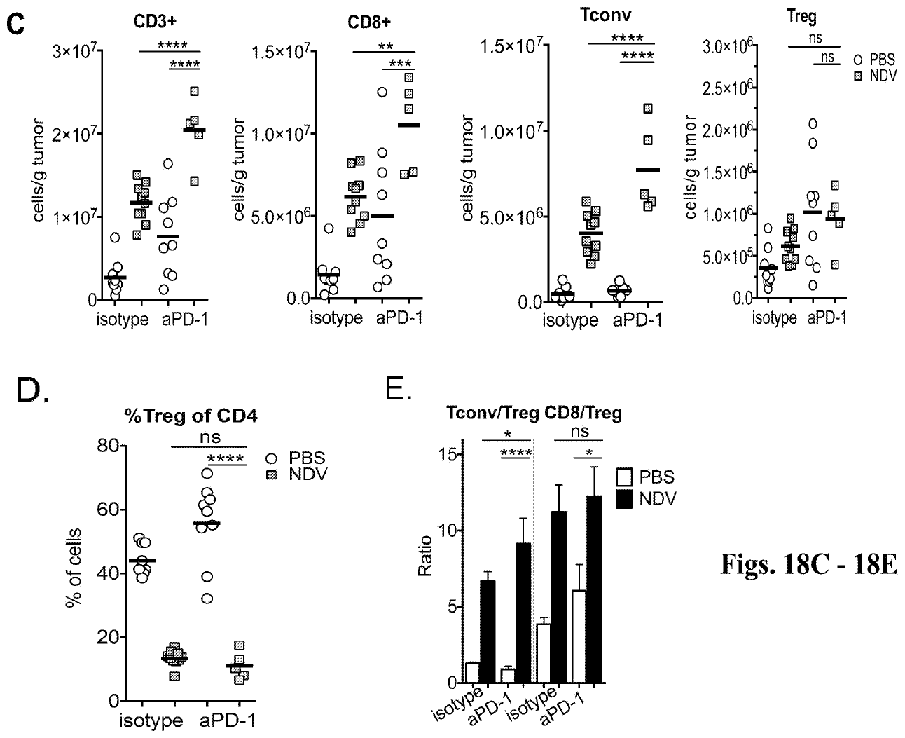

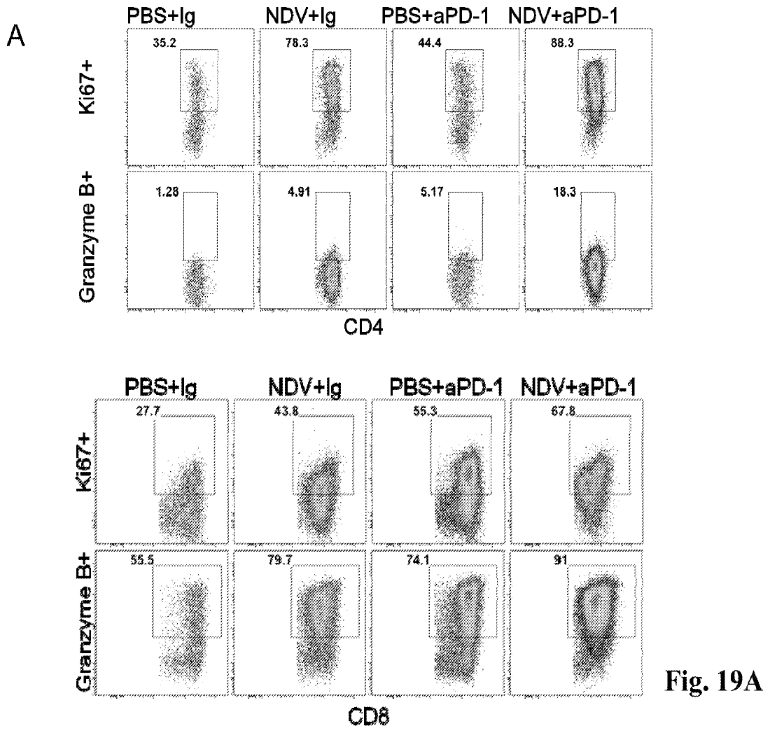

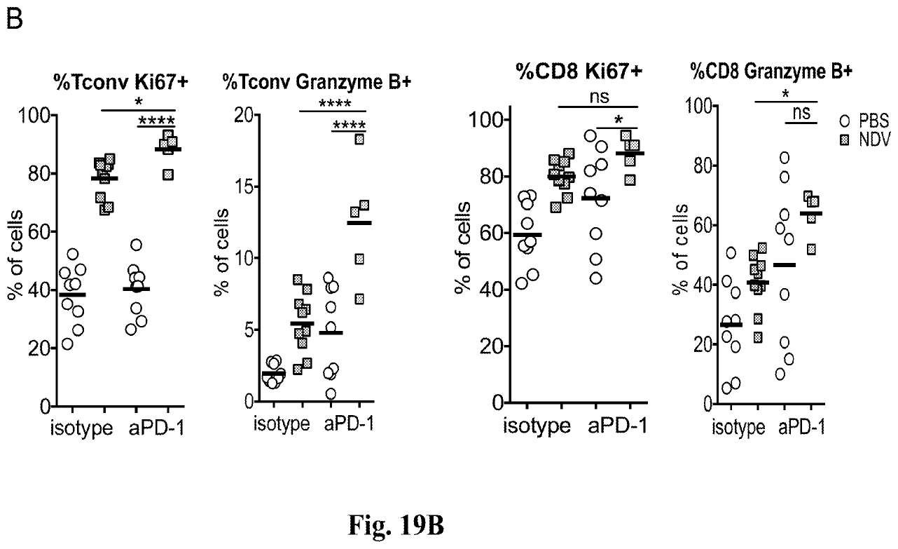

The combination of NDV with antibody blocking PD-1 and the combination of NDV with antibody blocking PD-L1 were evaluated in the bilateral flank melanoma model described above. NDV therapy in combination with either anti-PD-1 or anti-PD-L1 antibody led to improved animal survival (FIGS. 16A-16D and FIGS. 17A-17D). Distant tumors from animals treated with combination of NDV and anti-PD-1 antibody were characterized. As can be seen from FIGS. 18A-18E, combination of intratumoral NDV with systemic PD-1 blockade led to marked distant tumor infiltration with immune cells, with the increase in tumor-infiltrating CD8 cells being the most pronounced finding. The infiltrating cells upregulated proliferation and lytic markers Ki67 and granzyme B, respectively (FIGS. 19A-19B).

### 6.2.2.7 NDV Induces Tumor Immune Infiltration Upregulation of ICOS on CD4 and CD8 Cells in the Virus-Injected and Distant Tumors and Tumor Draining Lymph Nodes (TDLN)

The findings above demonstrated that combination of intratumoral NDV with systemic immune checkpoint blockade results in significant synergy between the two therapeutic approaches. To further build on these findings, enhancement of T cell effector function within the tumor microenvironment through a relevant co-stimulatory pathway may drive a better anti-tumor immune response was investigated. Previous studies identified the sustained upregulation of inducible costimulator (ICOS) on T cells as a strong indicator of response to CTLA-4 blockade in patients (Carthon et al., 2010, Clin. Canc. Res., 16:2861). ICOS is a CD28 homologue upregulated on the surface of activated T cells that has been shown to be critical for T cell-dependent B lymphocyte responses and development of all T helper subsets (Simpson et al., 2010 Curr Opin Immunol. 22:326). The role of ICOS in anti-tumor tumor efficacy of CTLA-4 blockade was recently confirmed by mouse studies, where ICOS-deficient mice were severely compromised in development of anti-tumor response with CTLA-4 blockade (Fu et al., 2011, Cancer Res., 71:5445).

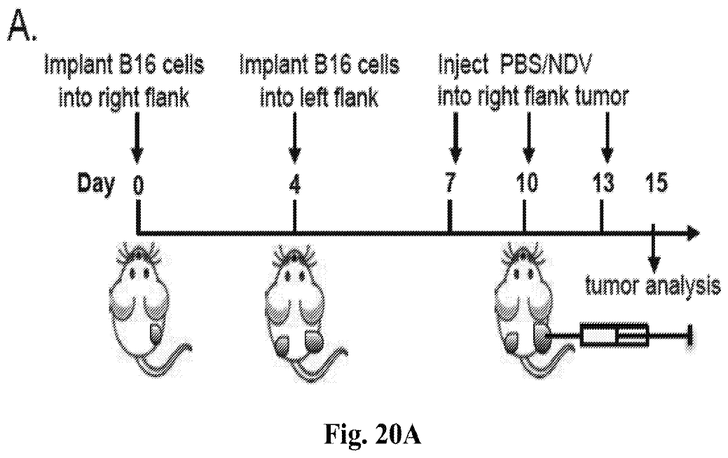

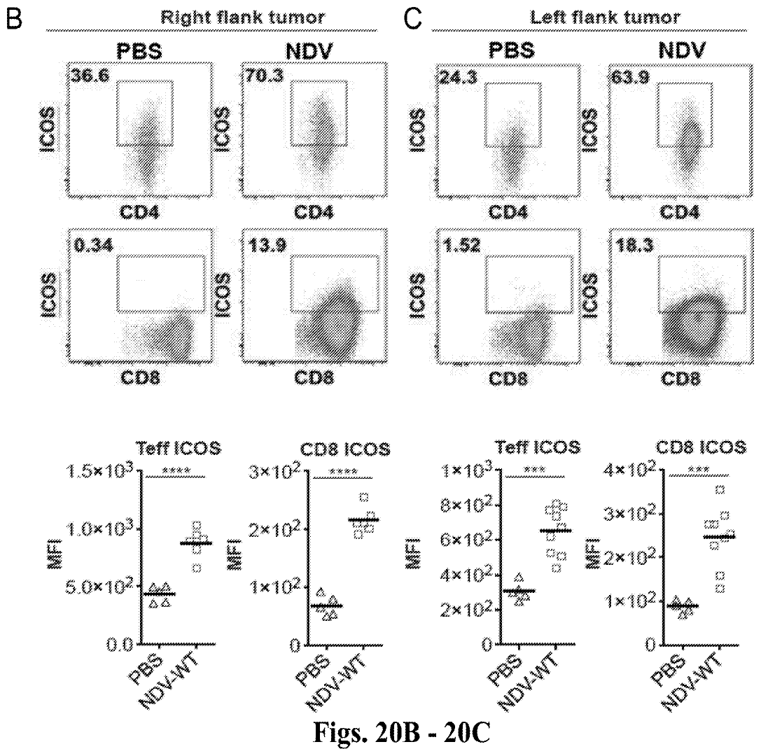

The expression of ICOS in bilateral flank tumor models treated with NDV were characterized to determine whether the receptor could serve as a target in this therapeutic approach. To characterize the local and abscopal effects of intratumoral NDV therapy, bilateral flank B16F10 melanoma models were utilized, with the virus administered to a unilateral tumor (FIG. 20A). Activation markers that could predict a better response and could be targeted for further improvement in therapeutic efficacy were assessed. The example focused on ICOS, as sustained ICOS upregulation has been previously been shown to be associated with more durable therapeutic responses and increased survival in patients treated with anti-CTLA-4 therapy for malignant melanoma. Analysis of lymphocytes isolated from the tumors and tumor-draining lymph nodes identified upregulation of the co-stimulatory molecule ICOS as one of the activation markers in the treated animals (FIGS. 20B and 20C).

### 6.2.3 Conclusion

To trigger immunogenic tumor cell death and an inflammatory response, nonpathogenic NDV was employed, which, despite its relatively weak lytic activity, has been demonstrated to be a potent inducer of type I IFN and DC maturation (Wilden et al., 2009, Int J Oncol 34:971; Kato et al., 2005, Immunity 23:19). A bilateral flank melanoma model with staggered implantation of tumors at a schedule that was previously demonstrated not to be affected by concomitant immunity was utilized (Turk et al., 2004, J Exp Med 200:771). This example demonstrates that intratumoral injection of NDV results in distant tumor immune infiltration in the absence of distant virus spread. Notably, this effect was associated with relative reduction in the number of Tregs and marked enhancement of CD4 and CD8 effector to Treg ratios, which has been previously demonstrated to be a marker of a favorable immunological response to immunotherapy (Quezada et al., 2006, J Clin Invest 116: 1935; Curran et al., 2010, Proc Natl Acad Sci USA 107: 4275).

The data in this example demonstrates that NDV enhances tumor infiltration with tumor-specific lymphocytes, an effect that was dependent on the identity of the virus-injected tumor. The enhanced tumor infiltration and expansion of adoptively-transferred lymphocytes further suggest the synergy between oncolytic virus therapy and therapeutic approaches utilizing adoptive T cell transfer. It is plausible that the tumor-specific lymphocytes undergo activation and expansion at the site of the initial viral infection, followed by their migration to other tumor sites, which is likely dependent on chemokines and lymphocyte homing receptors (Franciszkiewicz et al., 2012, Cancer Res 72: 6325). The data in this example also demonstrates that distant tumor immune infiltration was in part non-specific and could be induced by NDV infection of a heterologous tumor or by transfer of serum from treated animals to naïve tumor-bearing mice. Increased vascular permeability induced by inflammatory cytokines such as IL-6 may strongly contribute to activation of tumor vasculature and lymphocyte recruitment into the tumors (Fisher et al., 2011, The Journal of clinical investigation 121: 3846).

Despite the pronounced increase in TILs, therapeutic effect in distant tumors was rather modest with NDV monotherapy, highlighting the immunosuppressive nature of the microenvironment of these tumors (Spranger et al., 2013, Sci Transl Med 5).

In summary, this example demonstrates localized intratumoral therapy of B16 melanoma with NDV induces inflammatory responses leading to lymphocytic infiltrates and anti-tumor effect in distant (non-virally injected) tumors without distant virus spread. The inflammatory effect coincided with distant tumor infiltration with tumor-specific CD4+ and CD8+ T cells, which was dependent on the identity of the virus-injected tumor. This example demonstrates that localized therapy with oncolytic NDV induces inflammatory immune infiltrates in distant tumors, making them susceptible to systemic therapy with immunomodulatory antibodies.

### 6.3 Example 3

This example demonstrates that the intratumoral administration of NDV encoding IL-12 in combination with the administration of anti-PD-1 antibody results in robust anti-tumor activity in both NDV-IL-12 injected and non-injected tumors. As shown below, this anti-tumor activity correlates with the induction of immune genes and the infiltration of CD3+ T cells.

### 6.3.1 Materials and Methods

### 6.3.1.1 Cloning and Rescue of Viruses

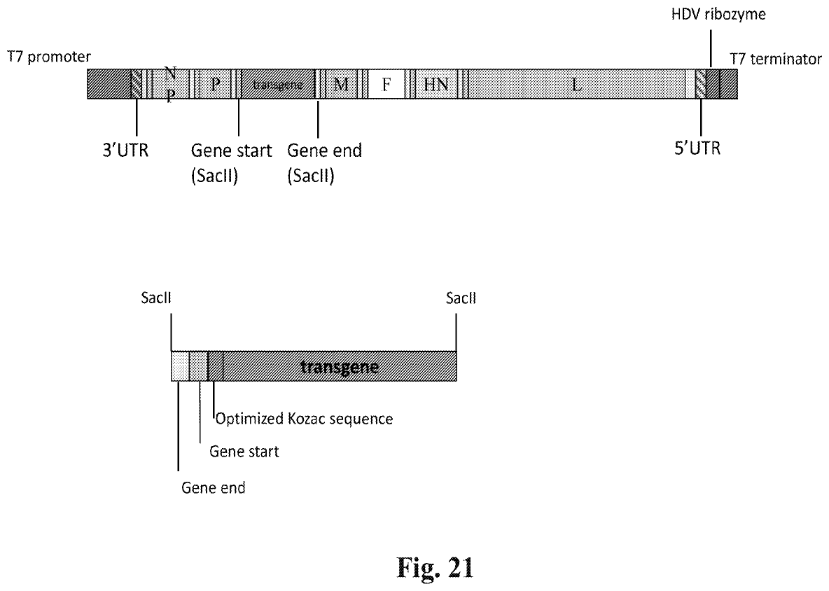

The NDV genome contains six open reading frames which encode the nucleoprotein (NP), phosphoprotein (P), matrix protein (M), fusion protein (F), hemagglutinin-neuraminidase (HN), and large viral polymerase protein (L). A human IL-12 transgene (SEQ ID NO: 26) consisting of nucleic acid sequences encoding the IL-12 p40 subunit, a GGGGGGS (SEQ ID NO: 24) linker, and the IL-12 p35 subunit was inserted into the SacII cloning site between the P and M genes in the pT7NDV-LS-L289A plasmid (FIG. 21; SEQ ID NO: 21). The resulting pT7NDV-LS-L289A plasmid containing the human IL-12 transgene (SEQ ID NO: 31) is referred to as “NDV-huIL-12” or “pT7NDV-LS-L289A-huIL-12” in this example. As shown in FIG. 21, the pT7NDV-LS-L289A plasmid contains the lentogenic NDV LaSota sequence under the transcriptional control of a T7 promoter. The plasmid contains an L289A mutation in the sequence encoding the NDV F protein. The L289A mutation in the NDV F protein has been reported to render the F protein more fusogenic as compared to the wild-type F protein, and promote oncolytic activity. See, e.g., Sergel et al., 2000, J. Virol. 74:5101; Altomonte et al., 2010, Mol. Ther. 18:275.

To generate the human IL-12 transgene for insertion into the pT7NDV-LS-L289A plasmid, forward and reverse primers (SEQ ID NOs: 32 and 33) were designed with the “Rule of Six” in mind and these primers were used to generate a PCR fragment with ends that have 15 base pairs of homology with the SacII cloning site in the pT7NDV-LS-L289A plasmid. Since successful rescue of NDV requires that the genome with the transgene follow the “Rule of Six”, the “Rule of Six” was considered during primer design and confirmed and corrected when necessary. The pT7NDV-LS-L289A plasmid was digested with SacII (NEB, Catalogue #R0157S) overnight at 37 degrees Celsius followed by Alkaline phosphatase (NEB, Catalogue #M0289S) treatment for 30 minutes at 37 degrees Celsius to prevent plasmid re-circularization. After digestion and alkaline phosphatase treatment, the linearized plasmid was purified using the NucleoSpin clean-up kit (Macherey-Nagel, Catalogue #740609.250). The PCR fragment and the cut plasmid were then infused together using the Infusion HD cloning kit from Takara Clontech. The resulting plasmids were transformed into Max Efficiency Stb12 Competent Cells (Life Technologies, Catalogue #10268-019). Positives clones were identified by colony PCR and sequence confirmation. For sequence confirmation, primers annealing outside of the cloned region were utilized (TCGATCGAGGAAATCAGG (SEQ ID NO: 36) and GTACAGCCCAATTGTCC (SEQ ID NO: 37). Thereafter, an endotoxin free midi prep was generated using the PureLink HiPure Midi prep kit (Invitrogen, Catalogue #K210004).

A similar strategy as described above with respect to the NDV-huIL-12 construct was utilized for the cloning and rescue of NDV encoding a murine IL-12 transgene (SEQ ID NO: 30). Namely, forward and reverse primers (SEQ ID NOS: 34 and 35) were designed with the “Rule of Six” in mind and the primers were used to generate a PCR fragment with ends that have 15 base pairs of homology with the SaclI cloning site in the pT7NDV-LS-L289A plasmid. The PCR fragment was cloned into the pT7NDV-LS-L289A plasmid using the techniques described above and positive clones were selected as described above. The pT7NDV-IS-L289A plasmid containing the murine IL-12 transgene (SEQ ID NO: 31) is referred to as “NDV-muIL-12,” or pT7NDV-LS-L289A-muIl-12 in this example.

Baby hamster kidney (BHK)-derived cells expressing T7 RNA polymerase (BSR-T7) were transfected with plasmids encoding L, NP, P and T7 RNA polymerase proteins and the pT7NDV-LS-L289A-huIL-12 plasmid or pT7NDV-LS-L289A-muIL-12 using the TransIT®-LT1 Transfection Reagent (Mirrus, Catalogue #MIR2300). In particular, for the rescue transfection, 30 μL of Trans-IT LT1 reagent and 150 μL of OptiMEM were used per well with the following plasmid concentrations (μg/well in a 6 well plate): 4.0 μg pT7NDV-LS-L289A-huIL-12 or pT7NDV-LS-L289A-muIL-12 plasmid; 2.0 μg of N plasmid, 1.0 μg of P plasmid; 1.0 μg of L plasmid; 2.0 μg of T7opt plasmid (Addgene plasmid #65974, obtained as a gift from Benhur Lee lab). To generate NDV-WT, rescue transfection was performed in a similar manner with pT7NDV-LS-L289A (i.e., the pT7NDV-LS-L289A-huIL-12 plasmid lacking the huIL-12 transgene). See, e.g., Ayllon et al., 2013, J. of Visualized Experiments, 80: e50830 for a description of the process for rescuing recombinant NDV from cDNA. The supernatant along with the cells were collected (using a cell scraper) 48 hours post-transfection and kept on ice until injected into the allantoic cavities of 9-10 day old embryonated chicken eggs. Injected eggs were incubated at 37 degree Celsius for 2 days, followed by overnight incubation at 4 degree Celsius. Allantoic fluid was then harvested. To harvest the allantoic fluid, the eggs' shells were cracked open under sterile conditions, the allantoic fluid was retrieved using a pipette and transferred into sterile falcon tubes kept on ice, and cellular debris were cleared by centrifugation (15×g). To confirm viral growth, a sample of this allantoic fluid was then used in a hemagglutination assay. The viruses were then aliquoted and directly frozen (−80 degrees Celsius) until use.

No observable difference in virus titer was observed with the introduction of the murine or human IL-12 transgene into the NDV. Representative virus titers are listed as follows after culture in eggs: 1.40×10⁸ for NDV-huIL12; 3×10⁸ for NDV-wt; and 1.7×10⁸ for NDV-muIL 12.

### 6.3.1.2 Hemagglutination (HA) Assay

HA assays were done using V-bottomed microtiter plates. 50 μL of 1×PBS was added to every well in the plate except for the first row. The first row received 100 μL of neat (i.e., undiluted) allantoic fluid carrying the virus. Two-fold serial dilutions of the virus suspension was then done across the entire row. Excess 50 μL was discarded after the last row. All wells had a final volume of 50 μL. 50 μl of 0.5% turkey erythrocyte suspension was then added to every well and the plate was tapped gently. The plate was incubated at 4 degrees Celsius for 20-30 minutes for the erythrocytes to settle. Samples showing complete hemagglutination were considered positive for virus. Negative results were observed as clear red pellets at the bottom of the plate.

### 6.3.1.3 IL-12 Expression

huIL-12 and muIL-12 expression was confirmed by commercially available kits from R&D systems-Human IL-12 Quantikine ELISA kits (D2050) and Mouse IL-12 Quantikine ELISA kits (M1270).

### 6.3.1.4 Analysis of NDV-huIL-12

NDV-huIL-12 was quantified and characterized by flow virometry using techniques described in Vlasak et al., Vaccine 34 (2016) 2321-2328, which is incorporated by reference herein in its entirety.

In addition, the expression of the six NDV proteins (F, HN, NP, L, P, and M) were analyzed by SDS-PAGE under reducing conditions. In particular, the SDS-PAGE analysis was executed using Invitrogen NuPAGE 4-12% Bis-Tris gels (ThermoFisher Scientific, Catalog No. NP0321BOX) in a MOPS (3-(N-morpholino) propanesulfonic acid)) buffer matrix. Samples were reduced using lithium dodecyl sulfate (LDS) and dithiothreitol (DTT) and heat treated for 10 minutes at 70° C. The gels were stained using GelCode Blue Stain (ThermoFisher Scientific, Catalog No. 24590) overnight, destained with water before imaged using a Molecular Dynamics Personal Densitometer SI and ImageQuant software.

Further, NDV titers were determined by a plaque assay with Vero cells in the presence of trypsin as described in Newcastle disease virus: propagation, quantification, and storage. Curr. Protoc Microbiol. 2006 June, Chapter 15: Unit 15F.2, which is incorporated by reference herein in its entirety.

### 6.3.1.5 Mice

WT C57BL/6 mice were purchased from Jackson Laboratory. All mice were maintained in microisolator cages and treated in accordance with the NIH and American Association of Laboratory Animal Care regulations. All mouse procedures and experiments for this study were approved by the Merck Research Laboratories (Boston and Palo Alto) Institutional Animal Care and Use Committee(s).

### 6.3.1.6 Cell Lines

The murine cancer cell line for melanoma (B16F10), was maintained in DMEM medium supplemented with 10% fetal calf serum.

### 6.3.1.7 Antibodies

Anti-mouse PD-1 monoclonal antibody (muDX400) and mouse IgG1 isotype control antibody (Mouse×[HEXON_Adenovirus] (TC31.27F11.C2) IgG1) were produced by Merck Research Laboratories.

### 6.3.1.8 Viruses and Cloning

Recombinant lentogenic NDV LaSota strain was used for all experiments. Viruses were rescued from cDNA using methods described previously and sequenced by reverse transcription PCR for insert fidelity. Virus titers were determined by serial dilution and immunofluorescence in Vero cells.

### 6.3.1.9 Tumor Challenge Experiments

Bilateral flank tumor models were established to monitor for therapeutic efficacy in both injected and systemic tumors. Treatment schedules and cell doses were established for each tumor model to achieve 10-20% tumor clearance by NDV or anti-PD-1 as single agents. For experiments evaluating combination therapy of either wild-type NDV (NDV-WT) or NDV expressing mouse IL-12 (NDV-muIL-12) with PD-1 blockade, B16F10 tumors were implanted by injection of 2×10⁵ and 1×10⁵ B16F10 cells subcutaneously (SC) in the right flank and left flank, respectively. Dosing was initiated 7-9 days following tumor implantation. Day 0 indicates first day of dosing. On Days 0, 2, 4 and 6 the mice were treated with 4 intratumoral injections of 1×10⁷ pfu of NDV-WT or NDV-muIL-12 in PBS in a total volume of 100 μl in the tumor in right flank (referred to as injected tumors). Tumors in left flanks are referred to as non-injected tumors. Concurrently, on Days 0, 4, and 8 or on Days 0 and 6 (depending on study design), mice received either 3 or 2 i.p. injections of anti-PD-1 antibody (10 mg/kg). Control groups received a corresponding dose of isotype antibody i.p. and intratumoral injection of PBS. Tumor size and incidence were monitored over time by measurement with a caliper. Following euthanasia of animals, tumors were either harvested and snap frozen using liquid nitrogen for RNA isolation and gene expression analysis or fixed in 10% neutral buffered formalin for 48 hour, transferred to 70% ethanol, and embedded in paraffin blocks for immunohistochemistry.

### 6.3.1.10 Immunohistochemistry

Mouse CD3 staining was performed on 4 μm thick formalin-fixed, paraffin-embedded tissue sections. The sections were deparaffinized and rehydrated in an ethanol series. Slides were subjected to heat-induced epitope retrieval and blocking of endogenous peroxidase before incubation with the primary antibody, anti-mouse CD3 rat mAb clone CD3-12 (AbD Serotec, Catalog No. MCA1477) at working dilution of 1:1000 for 60 mins at room temperature. Sections were then incubated with secondary antibody, Rabbit anti-Rat IgG H&L preadsorbed (Abcam, ab 102248) for 15 mins, followed by ImmPRESS™ HRP Anti-Rabbit IgG (Peroxidase) Polymer Detection Kit (Vector Laboratories, Catalog No. MP-7401) for 15 mins. Antigen-antibody binding was visualized with the use of 3,3-diaminobenzidine (Dako, Catalog No. K3468). Sections were counterstained with Mayer's Hematoxylin (Poly Scientific, Catalog No. S216-1GL), dehydrated, and cover-slipped.

### 6.3.1.11 Human Tumor Histoculture

Human tumor specimens from patients with renal cell carcinoma, colorectal carcinoma, breast carcinoma, non-small cell lung carcinoma, and head and neck squamous cell carcinoma were obtained from commercial sources (BioOptions, Boston BioSource (Tufts), Folio Biosciences, and Tissue Solutions) and academic collaborators (University of Rochester) in accordance with state and federal regulations. Fresh tumor tissues were collected within 1 hour following surgery to remove the tumors from the patients, placed in AQIX transportation medium and transported overnight at 4° C. to Merck Research Laboratories, Palo Alto, CA. The tumors were embedded in 1% low-melting gel and cut into 400 μm slices using the Mcllwain Tissue Chopper. The tumor slices were set onto Millicell-CM inserts in 6-well dishes containing 1 mL Dulbecco's modified Eagle's medium, 10% FBS, 100 U/mL penicillin, and 100 μg/mL streptomycin.

In the following experiments, 5 samples of renal cell carcinoma (RCC; n=4), 3 samples of colorectal carcinoma (CRC), 2 samples of breast carcinoma, and one sample of head and neck squamous cell carcinoma were evaluated. Alternatively, 6 samples of RCC, 1 sample of CRC, and 2 samples of non-small cell lung carcinoma (NSCLC) were evaluated. The tumor slices were incubated with 3×10⁷ pfu NDV-WT or NDV-huIL-12 or recombinant IL-12 at 10, 25 and 50 ng/mL and cultured at the air medium interface for 24 or 48 hours at 37° C. in an atmosphere with 5% CO₂. For certain experiments, the samples were snap-frozen (i.e., the samples were placed in tubes and dropped into liquid nitrogen until frozen and then transported on dry ice and stored at −80° C.) and RNA from the samples was isolated as described in Section 6.3.1.13, below.

For other experiments, media from the tissue samples was analyzed for cytokines and chemokines using various kits. GM-CSF, IFN-γ, IL-10, IL-12p70, IL-1-B, IL-2, IL-6, IL-8, and TNF-α were analyzed using the Human ProInflammatory 9-Plex Tissue Culture Kit (Meso Scale Discovery, Catalog No. K15007B-2). IFN-α-2a was analyzed using the Human IFN-α Tissue Culture Kit (Meso Scale Discovery, Catalog No. K151ACB-4); and IFN-β was analyzed using the Human IFN-β Tissue Culture Kit (Meso Scale Discovery, Catalog No. K151ADB-4). Eotaxin, Eotaxin-3, IL-8, IP-10, MCP-1, MCP-4, MIP-1a, MIP-1b, MDC, and TARC were analyzed using the V-PLEX Chemokine Panel 1 (human) Kit (Meso Scale Discovery, Catalog No. K15047D-2) and IP-10 was analyzed using IP-10 Tissue Culture Kit (Meso Scale Discovery, Catalog No. K151AVB).

### 6.3.1.12 Human Whole Blood Assay

Whole blood from patients with solid tumors (n=5) were obtained from Conversant Bio (Huntsville, AL), and whole blood from healthy donors (n=5) were obtained through the Merck Research Laboratories internal blood donor program. The following two experimental conditions were used. (1) 1 mL whole blood was left untreated or treated with 3×10⁷ pfu NDV-WT or NDV-huIL-12 for 48 hours. Plasma was collected for assessment of protein concentrations of various cytokines and chemokines using kits described above for the human tumor histoculture studies. (2) 4 mL whole blood was left untreated or treated with 3×10⁷ pfu NDV-WT or NDV-huIL-12 for 24 hours. Whole blood was collected into PAXgene blood RNA tubes (BD Biosciences Catalog No. 762165), and following RNA isolation, gene expression of panel of immune genes was analyzed using the Fluidigm RTqPCR platform (see Section 6.3.1.13).

### 6.3.1.13 Extraction of RNA and Real-Time Quantitative Polymerase Chain Reaction (RTqPCR)

RNA was isolated from mouse B16F10 tumors (Section 6.3.1.9), human tumor specimens (Section 6.3.1.11), and human whole blood (Section 6.3.1.12). Total RNA was isolated by homogenization into RNA STAT-60 (Tel-Test Inc., Friendswood, TX)) using a polytron homogenizer. The total RNA was extracted according to the manufacturer's protocol. After precipitation with isopropanol, total RNA was re-extracted with phenol: chloroform: isoamyl alcohol (25:24:1) using phase-lock light tubes.

DNase-treated total RNA was reverse-transcribed using QuantiTect Reverse Transcription (Qiagen, Catalog No. 2015310) according to manufacturer's protocol. Primers were obtained from Life Technologies:

Mouse genes

| Molecular | Life |
|---|---|
| Profiling | Technologies |
| Gene ID | Catalog # |
| Cd3-ε | Mm01179194_m1 |
| Cd8b1-CD8 | Mm00438116_m1 |
| Gzmb | Mm00442834_m1 |
| IL-12a-IL-12p35 | Mm00434165_m1 |
| IL-15 | Mm00434210_m1 |
| Ifn-γ | Mm01168134_m1 |
| Irf7 | Mm00516788_m1 |
| Mx1 | Mm00487795_mH |
| Oas1a | Mm00836412_m1 |
| Pdcd1-PD-1 | Mm00435532_m1 |
| Cd274-Pd-l1, B7h1 | Mm00452054_m1 |
| Ubb | Mm01622233_g1 |

Human genes

| Molecular Profiling | Life Technologies |
|---|---|
| Gene ID | Catalog# |
| CXCL9 | Hs00171065_m1 |
| Cxcl10 (Ip-10) | Hs00171042_m1 |
| CXCL11 | Hs00171138_m1 |
| DDX58 | Hs00204833_m1 |
| DHX58 | Hs00225561_m1 |
| GBP4 | Hs00925073_m1 |
| HERC6 | Hs00215555_m1 |
| IFIT1 | Hs00356631_g1 |
| IFIT2-ISG54 | Hs00533665_m1 |
| IFIT3 | Hs01922752_s1 |
| IFN-γ | Hs00989291_m1 |
| IL-12B-Il-12P40 | Hs01011518_m1 |
| IRF7 | Hs01014809_g1 |
| MX2 | Hs01550811_m1 |
| OAS3 | Hs00196324_m1 |
| OASl | Hs00388714_m1 |
| PARP14 | Hs00981511_m1 |
| CD274-PD-L1, B7H1 | Hs00204257_m1 |
| USP18 | Hs00276441_m1 |
| UBB | Hs00430290_m1 |

Real time quantitative polymerase chain reaction (RTqPCR) on 10 ng of cDNA from each sample was conducted using unlabeled primers at 900 nM each with 250 nM of FAM labeled probe in a TAQMAN™ RTqPCR reaction (Thermo Fisher Scientific, Foster City, CA) on the Fluidigm Biomark sequence detection system per manufacturer's protocol (Fluidigm, Foster City, CA). Levels of ubiquitin were measured in a separate reaction and were used to normalize the data by the Δ-Δ Ct method. Using the mean cycle threshold (Ct) value for ubiquitin and the gene of interest for each sample, the following equation was used to obtain the normalized values: 1.8 (Ct ubiquitin-Ct gene of interest)×10⁴.

### 6.3.1.14 Nanostring Gene Expression Analysis

For the analyses performed in Section 6.3.2.6, infra, RNA was quantified using the NanoDrop ND1000 spectrophotometer (Thermo Fisher Scientific). Gene expression analysis was conducted on the NanoString nCounter gene expression platform (NanoString Technologies). A custom code set consisting of a 800-gene panel related to T cell biology, immune regulation, and cellular markers of tumor-infiltrating lymphocytes and tumorassociated macrophages was used. Per sample, 50 ng of total RNA in a final volume of 5 μL was mixed with a 3′ biotinylated capture probe and a 5′ reporter probe tagged with a fluorescent barcode from the custom gene expression code set. Probes and target transcripts were hybridized overnight at 65° C. for 12-16 hours per the manufacturer's recommendations. Hybridized samples were run on the NanoString nCounter preparation station using the high-sensitivity protocol, in which excess capture and reporter probes were removed and transcript-specific ternary complexes were immobilized on a streptavidin-coated cartridge. The samples were scanned at maximum scan resolution on the nCounter Digital Analyzer. Gene expression data for each individual sample were normalized by HK (housekeeping) normalization. For each tumor sample, raw counts were log 10 transformed and then each gene normalized by subtracting off the arithmetic mean of all housekeeping genes (Table 15). Gene Expression Profiling (GEP) Signature scores were calculated as a weighted sum of the housekeeping normalized values of the 18 gene Up-Down Signature (Table 15). The house keeping normalized value for each gene was multiplied by the coefficient for that gene from the set of scoring weights to generate a weighted RNA value for each of the genes in the 18 gene signature, and adding the weighted RNA values to produce the signature score for the tumor sample. See Ayers et al., 2017, J of Clinical Investigation 127:2930-2940 and WO2016094377.

TABLE 15

| 18 Gene Up-Down Signature | | |
|---|---|---|
| Gene | Accession | Scoring Weight |
| Symbol | No. | Set |
| CCL5 | NM_002985.2 | 0.008346 |
| CD27 | NM_001242.4 | 0.072293 |
| CD274 | NM_014143.3 | 0.042853 |
| CD276 | NM_001024736.1 | −0.0239 |
| CD8A | NM_001768.5 | 0.031021 |
| CMKRLR1 | NM_004072.1 | 0.151253 |
| CXCL9 | NM_002416.1 | 0.074135 |
| CXCR6 | NM_006564.1 | 0.004313 |
| HLA.DQA1 | NM_002122.3 | 0.020091 |
| HLA.DRB1 | NM_002124.1 | 0.058806 |
| HLA.E | NM_005516.4 | 0.07175 |
| IDO1 | NM_002164.3 | 0.060679 |
| LAG3 | NM_002286.5 | 0.123895 |
| NKG7 | NM_005601.3 | 0.075524 |
| PDCD1LG2 | NM_025239.3 | 0.003734 |
| PSMB10 | NM_002801.2 | 0.032999 |
| STAT1 | NM_007315.2 | 0.250229 |
| TIGIT | NM_173799.2 | 0.084767 |

Housekeeping Normalization
Genes

| Gene | Accession |
|---|---|
| Symbol | No. |
| ABCF1 | NM_001090.2 |
| C14ORF102 | NM_017970.3 |
| G6PD | NM_000402.2 |
| OAZ1 | NM_004152.2 |
| POLR2A | NM_000937.2 |
| SDHA | NM_004168.1 |
| STK11IP | NM_052902.2 |
| TBC1D10B | NM_015527.3 |
| TBP | NM_001172085.1 |
| UBB | NM_018955.2 |
| ZBTB34 | NM_001099270.1 |

### 6.3.1.15 Cell Viability Assay

Cells (10,000/well/200 μL DMEM or RPMI supplemented with 10% FBS) were plated in replicates of 8 to 96-well plates and incubated at 37° C. in an atmosphere with 5% CO₂ for 18 hours to allow the cells to attach. Cell culture media was removed from wells prior to infection with NDV-huIL-12. In replicates of 8, cells were infected with NDV-huIL-12 at MOI 2 (20,000 pfu) or 6 (60,000 pfu) in 20 μL serum-free media (DMEM+0.3% BSA+penicillin/streptomycin). Media without virus (mock-infected) or media with 10 μM puromycin (or its vehicle DMSO) were added in parallel wells. Plates were incubated on a flat surface at room temperature for 1 hour, followed by aspiration of the media. Cell culture medium (100 μL) with 10% serum was added to each well, and the plates were incubated at 37° C. in an atmosphere with 5% CO₂ for 48 hours. Supernatants were collected and stored at −80° C. for analysis of cytokines and chemokines. The plates were then analyzed using a Cell Titer Glo assay kit (Promega, Catalog No. G7573) according to manufacturer's instructions.

### 6.3.1.16 Analysis of Cytokines and Chemokines from Human Tumor Cell Lines

The supernatants from the 26 cell lines that were infected with NDV-huIL-12 (MOI 2, replicates of 4) were assayed for various cytokines and chemokines. IL-10, IL-1B, IL-2, MCP-1, IL-6, IL-8, IL-10, IL-12p70, MIP-1b, and TNF-α were analyzed using a Human Custom Biomarker kit (Meso Scale Discovery, Catalog No. K15067L-2). IFN-α2a, IFN-β, IFN-γ, and IL-29/IFN-λ1 were analyzed using the Human Interferon Combo kit (Meso Scale Discovery, Catalog No. K15094K-2).

### 6.3.1.17 Statistics

Tumor Volumes: Follow-up of individual animals could be terminated early because of excessive tumor burden or other reasons. Depending on the reason and tumor size at the last measurement, the last observed tumor volume was treated as a lower bound on volume at all later days for that animal (right-censored data).

To compare two treatment groups on a given day, a generalization of the nonparametric Mann-Whitney (or Wilcoxon rank sum) test that allows for right-censored data was used: the Peto and Peto version of the Gehan-Breslow test. Two-sided p-values were estimated from 20,000 random reassignments of animals between the two treatments being compared. To control the familywise error rate across all time points for a given pair of treatments, p-values were multiplicity adjusted by Holm's method. A p-value of less than 0.05 was used to define statistical significance.

Gene expression data from tumor challenge studies: To compare two treatment groups on a given day, nonparametric Mann-Whitney test was used. A p-value of less than 0.05 was used to define statistical significance.

Cytokine/chemokine and/or gene expression data from human tumor histoculture and whole blood studies: To compare two treatment groups on a given day, nonparametric Wilcoxon signed rank test was used. A p-value of less than 0.05 was used to define statistical significance.

### 6.3.1.18 IL-12 Bioassay

To evaluate the functionality of huIL-12 produced from NDV-huIL-12 in NDV-huIL-12-infected cells, Vero cells were seeded in 6-well tissue culture plates at 5×10⁵ cells/well and incubated at 37° C., 5% CO₂ for 24 hours. Test samples of NDV-huIL-12 viruses were diluted to 1×10⁶ pfu/mL in Opti-MEM (1×) reduced serum medium and various amounts of the diluted samples were transferred to the cell plate to target MOI between 0.03-1. Opti-MEM (1×) reduced serum medium was then added to each well to a final volume of 2 mL. The infected cell plate was incubated at 37° C., 5% CO₂ for 24 hours. The function of the produced hull-12 in the supernatant was assayed using the PathHunter® Bioassay Detection kit (DiscoverX, Cat #93-0933).

PathHunter® U2OS IL12RB1/IL12RB2 cells were engineered to co-express human IL-12 receptor beta 1 and beta 2. One receptor was fused to Enzyme Donor (ED), and the other receptor was fused to Enzyme Acceptor (EA). Upon binding of functional huIL-12, the two receptors dimerize, forcing complementation of ED and EA to form a functional β-galatosidase that hydrolyzes a substrate to generate a chemiluminescent signal. U2OS IL 12RB1/IL 12RB2 cells were seeded in a 96-well cell plate at 5×10³ cells/well and incubated at 37° C., 5% CO₂ for 4-6 hours. A total of 60 μL supernatant fluids from each well of the infected Vero plate were transferred to the second column of a 96-well sample dilution plate and a 3-fold serial dilution in AssayComplete Cell Plating Reagent (DiscoverX, 93-0563R5A) was carried out across rows. A total of 10 μL, of each diluted supernatant was transferred to the U2OS cell plate and the plate was incubated at 37° C., 5% CO₂ for 16-20 hours. 10 μL of Detection Reagent 1 was then added to each well and the plate was incubated for 15 minutes at room temperature. 40 μL of Detection Reagent 2 was then added to each well and the plate was further incubated for 60 minutes at room temperature. The chemiluminescence signal was detected using SpectraMax M5 plate reader.

### 6.3.1.19 Quantitation of huIL-12 by ELISA

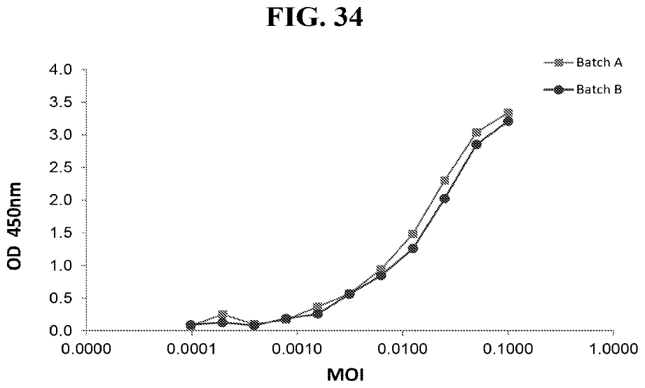

Vero cells were seeded in 96-well tissue culture plates at 1×10⁴ cells/well in Opti-PRO serum free medium (Gibco, Cat. No. 12309-019) supplemented with 2% glutamine (Corning, Cat. No. 25-005-CI) and incubated at 37±2° C., 5±2% CO₂ for approximately 24 hours. Test samples of NDV-huIL-12 viruses (batches A and B) were pre-diluted in Opti-MEM (1×) reduced serum medium (Gibco, Cat. No. 31985-070) to target 2×10⁴ pfu/mL. A total of 300 μL of each pre-diluted test sample was added to the first row of a 0.5 mL Assay Block (Costar, Cat. No. 3956) and a 2-fold serial dilution was performed across rows by transferring 150 μL of sample into 150 μL Opti-MEM (1×) reduced serum medium. Two replicates per sample were prepared. The 96-well plates containing Vero cells were removed from the incubator approximately 24 hours post seeding and spent medium is removed from the plate. The cells were inoculated with 100 μL serially diluted test samples and incubated at 37° C., 5% CO₂. After approximately 24 hours incubation, 90 μL of supernatant fluid was removed from the infection plates, transferred to an ELISA plate pre-coated with anti-human IL-12 p70 capture antibody (Affymetrix eBioscience, Cat. No. 14-7128-68) and incubated for two hours at room temperature. The captured hulL-12 was detected with anti-human IL-12 p70 detection antibody (Affymetrix eBioscience, Cat. No. 33-8261-68A) and Avidin-HRP and visualized with a HRP substrate TMB following vendor's procedure (Affymetrix eBioscience, Human IL-12 p70 ELISA Ready-SET-Go! ELISA kit, Cat. No. 88-7126-88). The MOI-dependent huIL-12 expression curve was compared to a NDV-huIL-12 reference standard (FIG. 34).

### 6.3.1.20 Purification

Purification of NDV-huIL-12 from harvested allantoic fluid uses a process consisting of the following unit operations: (1) clarification, (2) tangential flow filtration, and (3) sterile filtration. Harvested allantoic fluid was clarified using 1.2 μm glass fiber dead end filtration to remove cells and other large debris. The membrane that was used for this step contains a net positive charge which results in a measurable reduction in host cell DNA. The clarified bulk (CB) was subsequently processed by tangential flow filtration (TFF) using 750 kD hollow fiber membranes. During this step the batch was concentrated ˜5-fold and diafiltered against 4 diavolumes (DVs) of high salt buffer followed by 4 DVs of low salt formulation buffer and then concentrated ˜10-fold. This unit operation constitutes the main purification step whereby residual egg proteins, primarily ovalbumin, and residual DNA are reduced to acceptable levels. The step also serves to exchange buffer to one compatible with formulation. The TFF Product (TFFP) was then sterile filtered through a 0.2 μm filter and dispensed into appropriate sterile storage containers. The purified virus bulk (PVB) was then frozen and stored at −70 degrees Celsius.

### 6.3.2 Results

### 6.3.2.1 Anti-Tumor Efficacy and Mechanism of Action of NDV-muIL-12 in Combination with Anti-muPD-1 mAb in Syngeneic Mouse B16F10 Bilateral Tumor Model

B16F10 tumors are poorly infiltrated with T cells and resistant to anti-PD-1 therapy. B16F10 tumors display very aggressive growth and represent a high-bar model. Therefore, the B16F10 model was selected to evaluate NDV vectors encoding the mouse IL-12 transgene in comparison to unarmed NDV (“NDV wild-type” (“NDV-WT”)). The NDV vectors were evaluated in two-tumor bearing C57BL/6 mice to evaluate the abscopal effect (i.e., anti-tumor efficacy in the tumor that was not administered NDV).

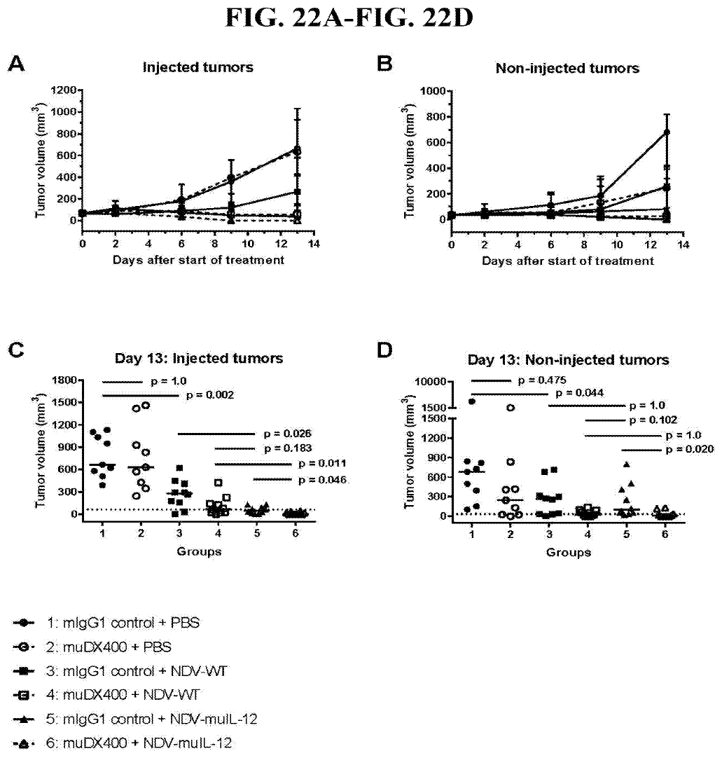

Compared to mouse IgG1 isotype control (Group 1), administration of muDX400 alone (Group 2) did not result in significant reduction in tumor volumes of either injected tumors (p=1.0) or non-injected tumors (p=0.475) (FIG. 22). Compared to NDV-WT (Group 3), administration of NDV-muIL-12 alone (Group 5) resulted in significant reduction in tumor volumes of injected tumors (p=0.027) but not of non-injected tumors (p=1.0) (FIG. 22). However, compared to NDV-muIL-12 (Group 5), NDV-muIL-12 in combination with muDX400 (Group 6) resulted in significant reduction in tumor volumes of both injected tumors (p=0.047) and non-injected tumors (p=0.020) (FIGS. 22A-22D). In addition, NDV-muIL-12 in combination with muDX400 resulted in 6 out of 10 complete regressions (CRs) and 5 out of 10 CRs in injected tumors and non-injected tumors, respectively (FIGS. 22A-22D). These results were reproducible in an independent experiment, are consistent with previous findings demonstrating that in mouse pre-clinical models, NDV breaks resistance to immune checkpoint blockade (Zamarin et al., 2014, Sci. Transl. Med. 6: 226ra32 (2014)). For injected tumors, the number of complete regressions were as follows: Group 1:0; Group 2:0; Group 3:1; Group 4: 1; Group 5:2; and Group 6:6. For non-injected tumors, the number of complete regressions were as follows: Group 1:0; Group 2:1; Group 3:1; Group 4:3; Group 5:0; and Group 6:5.

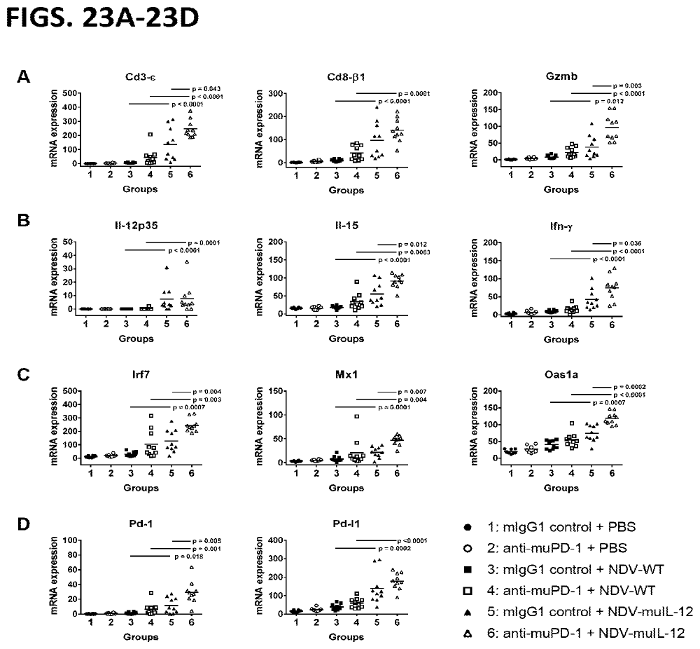

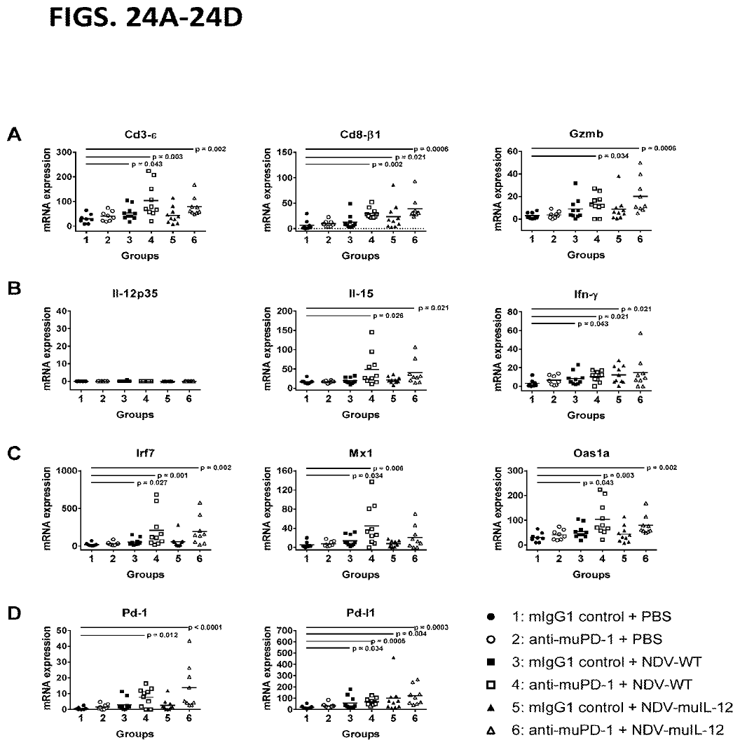

In the same study, tumors were harvested on Day 14 to evaluate expression of various immune genes. FIG. 23 and FIG. 24 show mRNA expression of representative genes for T cell markers (FIG. 23A and FIG. 24A), cytokines (FIG. 23B and FIG. 24B), IFN-inducible genes (FIG. 23C and FIG. 24C), and PD-1 pathway (FIG. 23D and FIG. 24D) in injected tumors and non-injected tumors, respectively. mRNA expression of Il-12p35 is only detected in injected tumors of animals which were administered NDV-muIL-12 (Groups 5 and 6). This result suggests that NDV itself did not induce IL-12. Also the lack of detection of mRNA expression of Il-12p35 in the non-injected tumors of Groups 5 and 6 supports previous findings that NDV is restricted to the NDV injected tumors (Zamarin et al., 2014, Sci. Transl. Med. 6: 226ra32 (2014). As shown in FIGS. 23A-23D, mRNA expression of CD3-c, CD8-B, Gzmb, IL-12p35, IL-15, IFN-γ, Irf7, Mx1, Oasla, PD-1, and PD-L1 in the injected tumors increased with the following order of treatments: control (Group 1), muDX400 monotherapy (Group 2), NDV-WT monotherapy (Group 3), combination of muDX400 and NDV-WT (Group 4), NDV-muIL-12 monotherapy (Group 5), and combination of muDX400 and NDV-muIL-12 (Group 6), and the increase is statistically significant (p<0.05) between Groups 3 and 5 and Groups 4 and 6. Increases of these genes in the non-injected tumors are stastically significant between control (Group 1) and combination of muDX400 with either NDV-WT (Group 4) or NDV-muIL-12 (Group 6) (FIGS. 24A-24D). Similar results were obtained from an independent experiment.

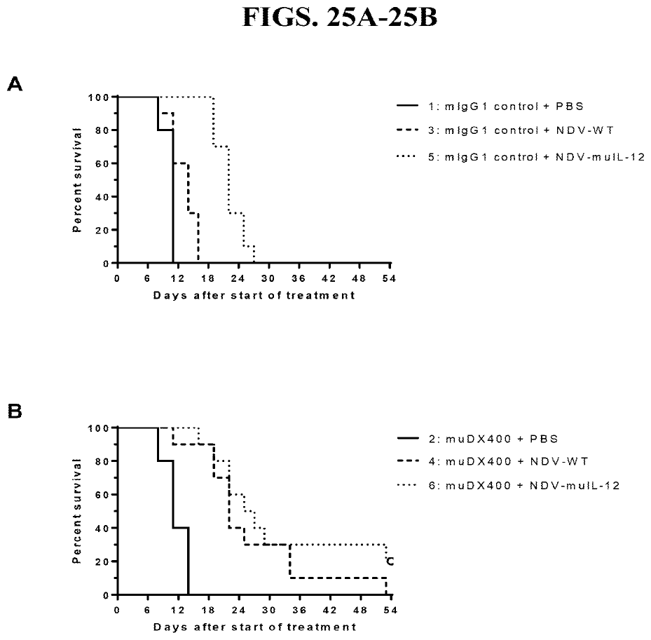

In another independent bilateral B16F10 study, mouse B16F10 cells were subcutaneously implanted into the right flanks (2×10⁵ cells) and left flanks (1×10⁵ cells) of immunocompetent C57BL/6J mice. Animals were assigned into 6 groups 7 days after implantation (Day 0) based on tumor volume (TV) in the right flanks (injected tumors) with median TV-56 mm³. The median TV in the left flanks (non-injected tumors) was 38 mm³. Dosing was initiated on Day 0. Mouse IgG1 isotype control and muDX400 at 10 mg/kg were administered intraperitoneally every 4 days for a total of 3 doses. PBS, NDV-WT, and NDV-muIL-12 at 1×10⁷ pfu were administered into the tumors on the right flanks every 2 days for a total of 4 doses. Animals were followed up to Day 54. Animals were euthanized when the sum of volumes of injected and non-injected tumors was >2000 mm³ or body weight loss was ≥20%. There were 10 animals in each group. As shown in FIGS. 25A-25B, treatment with intratumoral NDV-muIL-12 in combination with muDX400 resulted in longest overall survival of the animals.

In another bilateral B16F10 study, mouse B16F10 cells were subcutaneously implanted into the right flanks (2×10⁵ cells) and left flanks (1×10⁵ cells) of immunocompetent C57BL/6J mice. Animals were assigned into 6 groups 9 days after implantation (Day 0) based on tumor volume (TV) in the right flanks (injected tumors) with median TV=108 mm³. The median TV in the left flanks (non-injected tumors) was 83 mm³. Dosing was initiated on Day 0. Mouse IgG1 isotype control and muDX400 at 10 mg/kg were administered intraperitoneally every 4 days for a total of 2 doses. PBS, NDV-WT, and NDV-muIL-12 at 1×10⁷ pfu were administered into the tumors on the right flanks every 2 days for a total of 4 doses. Injected and non-injected tumors (n=3/group) were harvested on Day 8, and CD3 expression analyzed by immunohistochemistry. As shown in FIGS. 26A-26B, treatment with intratumoral NDV-muIL-12 in combination with muDX400 resulted in a strikingly high infiltration of CD3+ T cells in both the injected and non-injected tumors. Flow cytometry analysis suggested that the majority of the infiltrating CD3+ cells are activated CD8 or CD4 effector cells (not shown).

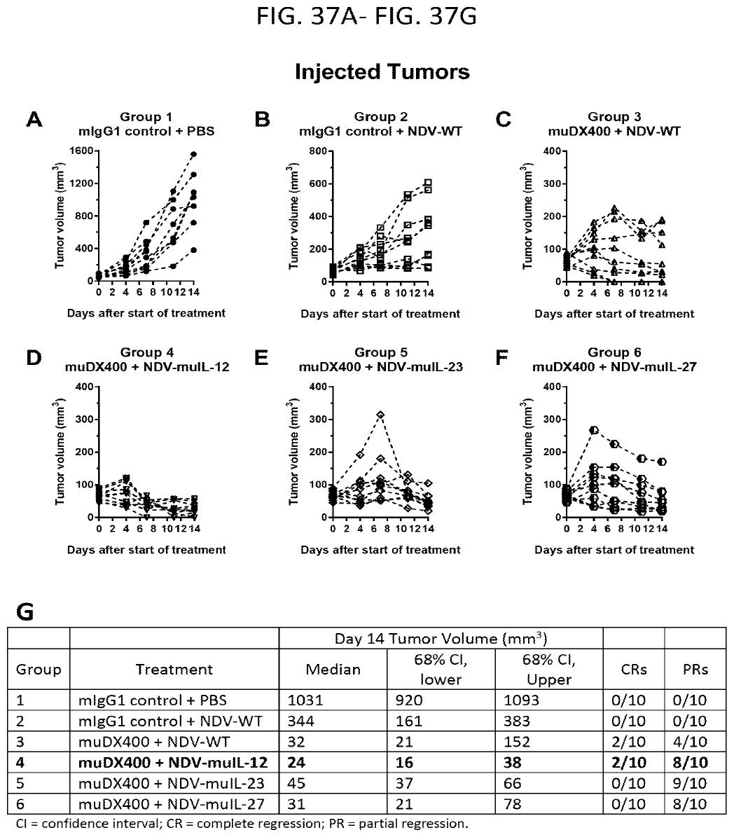

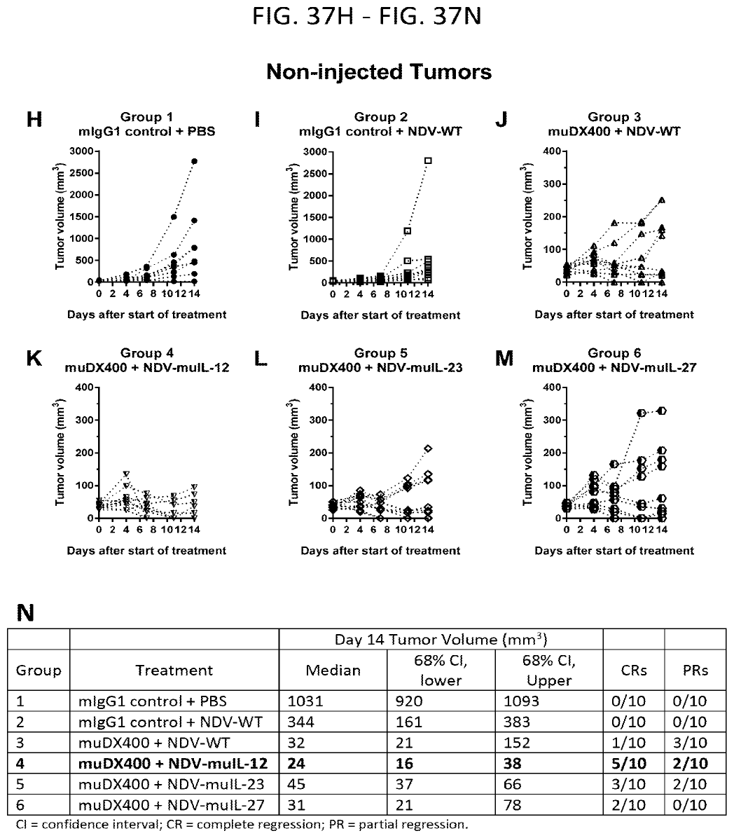

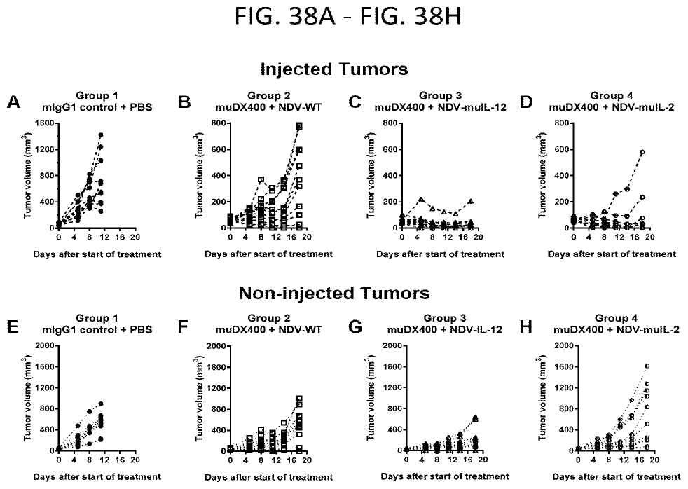

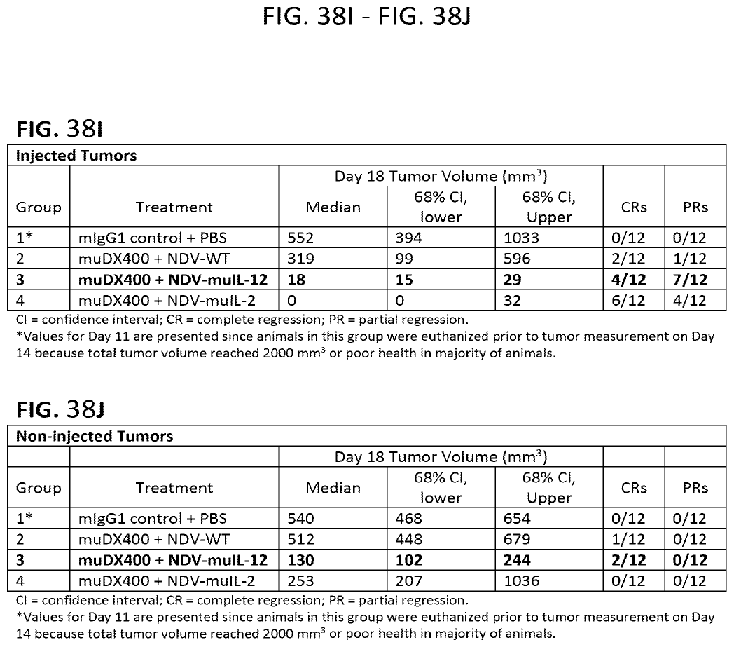

In addition, the anti-tumor efficacy of arming NDV with IL-12 versus other cytokines (IL-23, IL-27, and IL-2) in combination with anti-muPD-1 monoclonal antibody (muDX400) in the bilateral B16F10 was assessed. In the study comparing NDV-muIL-12 with NDV-muIL-23 and muIL-27, in combination with muDX400, treatment with NDV-muIL-12+muDX400 resulted in the highest number of regressions (complete and partial) in injected and non-injected tumors (FIGS. 37A-37N) but there was not a significant reduction in tumor volumes for either injected or non-injected tumors (p>0.05). In this study, the combination of NDV-muIL-12 and muDX400 resulted in a higher number of complete regressions in injected tumors and non-injected tumors than the combination of anti-muDX400 and NDV-muIL-23 or NDV-muIL-27 (FIGS. 37G and 37N). Further, in the study comparing NDV-muIL-12 with NDV-muIL-2, in combination with muDX400, the effect on the injected tumors with the two treatments were comparable resulting in comparable number of complete and partials regressions and comparable median TV and 68% confidence interval at Day 18 (see FIGS. 38C, 38D, and 38I). For the non-injected tumors (FIGS. 38G, 38H, and 38J), although the reduction in tumor volume at Day 18 was not significant between the two treatments (p>0.05), NDV-muIL-12+muDX400 resulted in median TV=130 mm³ and tighter 68% confidence interval (102, 244 mm³) compared to NDV-muIL-2+muDX400: median TV=255 with 68% confidence interval (207, 1036 mm³). In this study, the combination of NDV-muIL-12 and muDX400 resulted in a higher number of complete regressions in non-injected tumors than the combination of muDX400 and NDV-muIL-IL-2 (FIG. 38J).

### 6.3.2.2 Lytic Activity of NDV-huIL-12 in Human Tumor Cell Lines

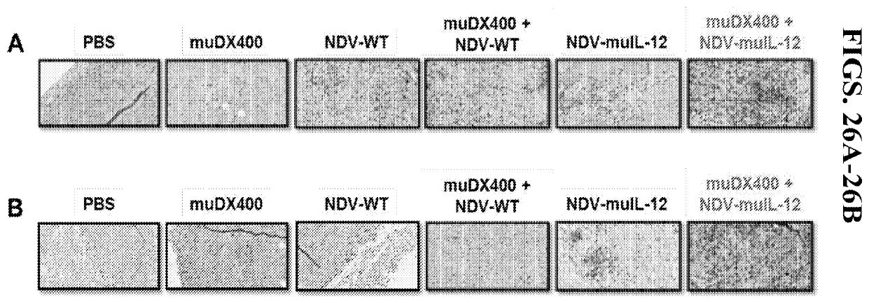

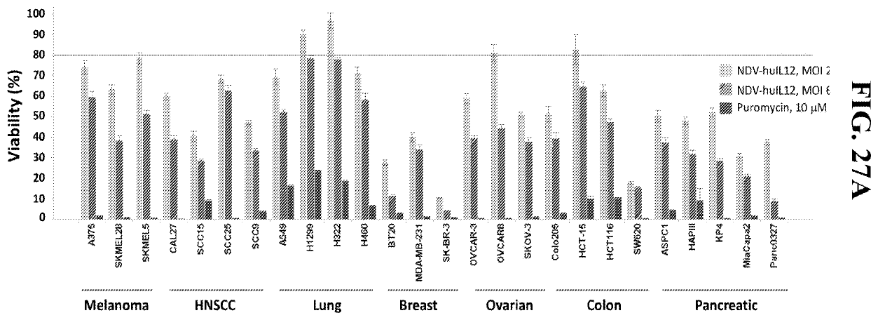

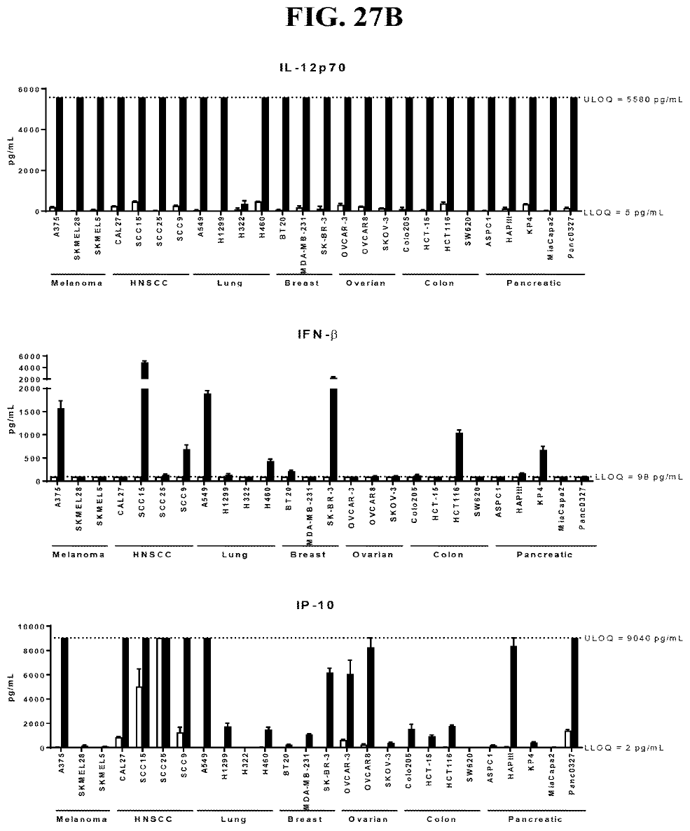

The lytic activity of NDV-huIL-12 was assessed in a panel of 26 human cancer cell lines at multiplicity of infection (MOI) of 2 and 6, 48 hours following infection (FIG. 27A). The panel included 7 cancer types: melanoma, HNSCC (head and neck squamous cell carcinoma), lung, breast, ovarian, colon, and pancreatic carcinomas. Cell viability was assessed by an ATP-based quantification method 48 hours after virus infection (FIG. 27A). For each cell line, lytic activity of NDV-huIL-12 (MOI 2 and 6) was expressed as a percentage of viability relative to mock-infected cells; lytic activity of 10 μM puromycin (positive control) was expressed as a percentage of viability relative to vehicle (DMSO) treated cells.

With a monobasic F protein cleavage site, NDV-huIL-12 is limited to a single cycle of replication in tumor cells. With this consideration, the criterion for sensitivity to NDV-huIL-12 was set at >20% reduction in cell viability with MOI of 2. By this criterion, 22 out of the 26 cell lines were sensitive to lysis by NDV-huIL-12. At a MOI of 6, all cell lines were sensitive to lysis by NDV-huIL-12 (>20% reduction in cell viability). In conclusion, lytic activity of NDV-huIL-12 was demonstrated in range of human tumor cell lines.

### 6.3.2.3 Induction of Cytokines and Chemokines in Human Tumor Cell Lines with NDV-huIL-12 Treatment

The cell culture supernatants from the 26 human tumor cell lines that were assayed for lytic activity of NDV-huIL-12 (MOI 2) or mock infected were harvested at 48 hours and analyzed for various cytokines and chemokines by immunoassays. Mean concentrations of IL-12p70, IFN-β, and IP-10 are presented in FIG. 27B. NDV-huIL-12 induced secretion of levels of IL-12p70 that were above the upper limit of detection in the immunoassay in all the cell lines except lung carcinoma cell line H322. NDV-huIL-12 did not induce IFN-γ in any of the cell lines (data not shown); this is expected as IL-12 receptor is mainly expressed by activated T cells and NK cells. NDV-huIL 12 induced IFN-α-2a in only 2 cell lines (breast SK BR-3 an HNSCC SCC15; data not shown) but induced IFN-β in a third of the cell lines. In addition, NDV-huIL-12 induced moderate/high levels of IP-10 in 20 cell lines.

### 6.3.2.4 Induction of Immune Genes in Human Tumor Histoculture and Whole Blood with NDV-huIL-12 Treatment

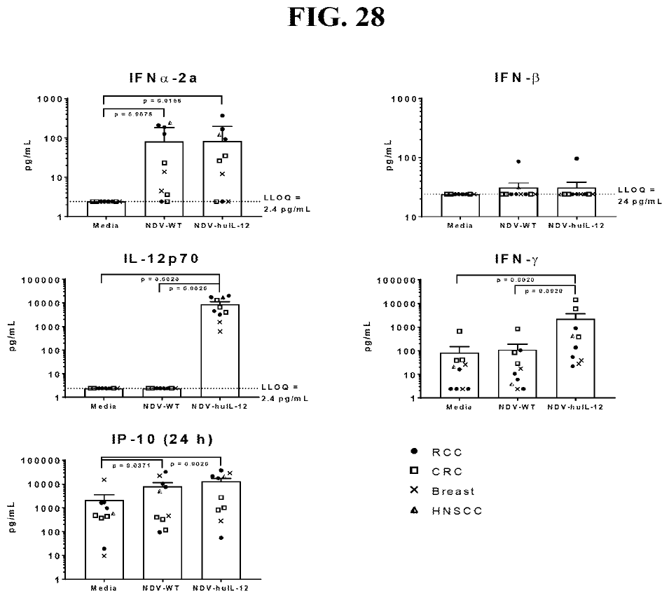

The human tumor histoculture approach allows culturing of intact fresh cancer tissues in the presence of drug for up to 48 hours. The effect of drugs on cancer cells and pre-existing immune cells in the tumor specimens can be assessed. Induction of cytokines and chemokines (protein expression) and immune genes (gene expression) by NDV-WT and NDV-huIL-12 was evaluated in this platform. In these studies, 4 samples of renal cell carcinoma (RCC), 3 samples of colorectal carcinoma (CRC), 2 samples of breast carcinoma, and 1 sample of HNSCC were treated with 3×10⁷ pfu NDV-WT or NDV-huIL-12 for up to 48 hours. At the 24-and 48-hour time points, supernatants from the histoculture were collected and analyzed for various cytokines and chemokines by immunoassays, and tumors were snap-frozen and RNA was isolated for analysis of immune genes. Mean concentrations of IFN-α-2a, IFN-β, IL-12p70, and IFN-γ, and IP-10 are presented in FIG. 28, and gene expression of IFN-inducible genes IRF7, IFIT2, and MX2, chemokines CXCL9, CXCL10 (IP-10), and CXCL11, IL-12P40, IFN-γ, and PD-L1 are presented in FIGS. 29A-29D. Both NDV-WT and NDV-huIL-12 induced secretion of IFN-α-2a and IP-10; neither induced secretion of IFN-β. Only NDV-huIL 12 induced IL-12p70 and IFN-γ. The analysis of gene expression of various immune genes showed that NDV-huIL-12 induced strong type 1 IFN and IL-12 responses along with induction of chemokines and PD-L1 in multiple tumor types.

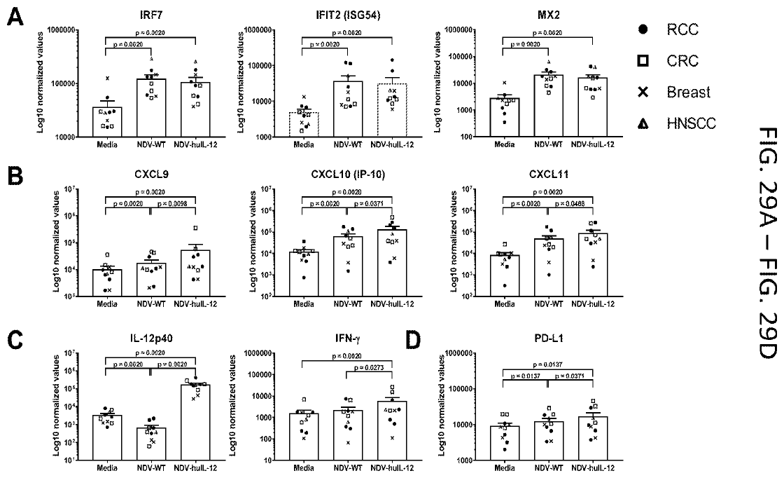

Similarly, induction of cytokines and chemokines (protein expression) (FIG. 30) and immune genes (gene expression) (FIGS. 31A-31D) by NDV-WT and NDV-huIL-12 was evaluated in whole blood from both patients with solid tumor malignancies (n=5) and normal donors (n=5). Although cancer cells may be present in the whole blood from the cancer patients, the pattern of immune activation with NDV-WT and NDV-huIL-12 is comparable between blood from both sets of donors. The pattern of induction of IFN-α-2a, IFN-β, IL-12p70, and IFN-γ, and IP-10 and gene expression of IFN inducible genes, chemokines, Il-12p40, IFN-γ, and Pd 11 were similar to that observed in the human tumor histoculture, suggesting that NDV-huIL-12 is in part acting on the immune cells in the intact tumor tissues.

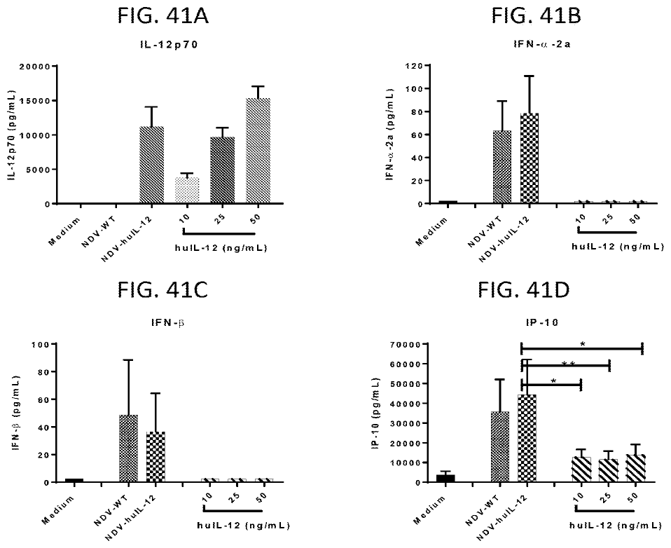

Type I IFN pathway has emerged as an important regulator of anti-tumor immune response, with several studies demonstrating its role in antigen presentation and dendritic cell maturation (see, e.g., Zitvogel et al., Nature reviews. Immunology 15, 405-414 (2015), Fuertes et al., J Exp Med 208, 2005-2016 (2011), Diamond et al., J Exp Med 208, 1989-2003 (2011), Kato et al., Immunity 23, 19-28 (2005)). In the human tumor histoculture platform, treatment of tumors with 3×10⁷ pfu NDV-huIL-12 resulted in secretion of levels of IL-12 (11189±2877 pg/mL) that is in range with treatment with 25 ng/ml (9636±1405 pg/mL) and 50 ng/ml (15308±1726 pg/mL) of recombinant IL-12 (FIG. 41A-41D). However, only treatment with NDV-huIL-12 but not recombinant IL-12 (10 to 50 ng/ml) induced IFN-α-2a and IFN-β in most of the donors tested (FIG. 41A-41D). In addition, treatment with NDV-huIL-12 induced secretion of significantly higher levels of IP-10 which plays an important role in T cell recruitment to the tumor site (FIG. 41A-41D).

### 6.3.2.5 Identification of NDV-huIL-12 Response Signature

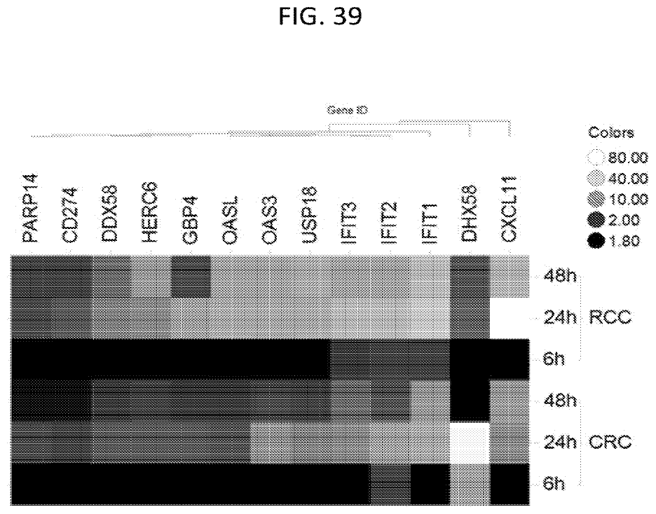

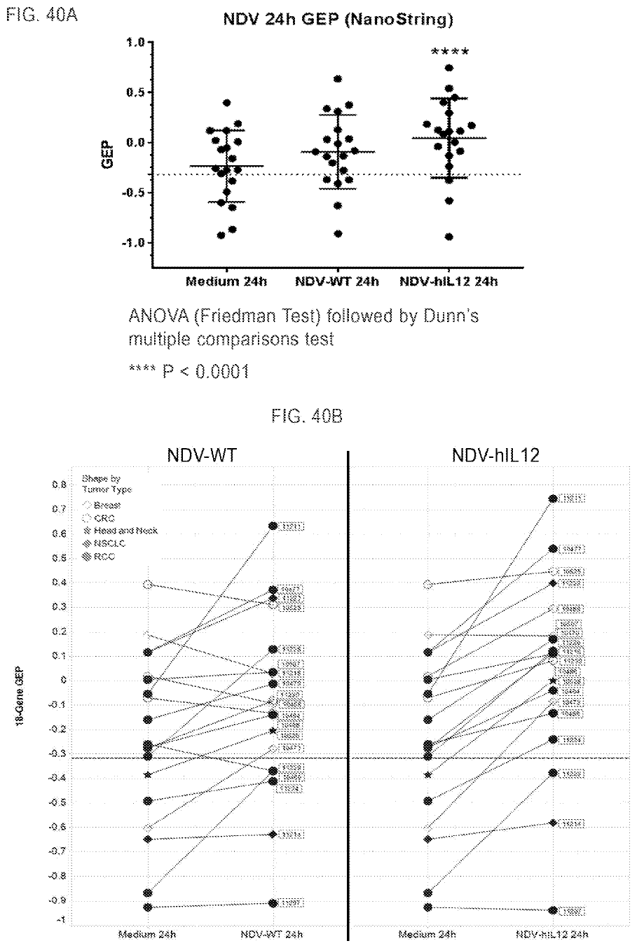

The human tumor histoculture platform was utilized to identify genes that were modulated at least 2-fold, p<0.01, following treatment with 3×10⁷ pfu NDV-huIL-12 for up to 48 hours. Gene expression was analyzed by RNA sequencing. The response signature that includes interferon-inducible genes (e.g., GBP4, IFIT1, IFIT2, IFIT3, OAS3, and OASL), chemokines (e.g., CXCL10 and CXCL11), and PD-L1 was confirmed in additional tumor samples (n=14) by RTqPCR analysis (FIG. 39).

### 6.3.2.6 Induction of NDV-huIL-12 Response-Signature in Both GEP-Negative and GEP-Positive Tumors

Using RNA from baseline tumor samples of anti-PD-1 pembrolizumab-treated patients, an immune-related gene-expression profile (GEP) correlating with clinical benefit with pembrolizumab was identified (Ayers et al., 2017, J of Clinical Investigation 127:2930-2940). The T cell-inflamed GEP contained IFN-γ-responsive genes related to antigen presentation, chemokine expression, cytotoxic activity, and adaptive immune resistance. Patients who are GEP-negative, rarely respond to pembrolizumab. It is demonstrated in the human tumor histoculture platform that the increase in GEP score is higher for NDV-huIL-12 compared to NDV-WT for treatment of tumors (n=19). In addition, treatment with 3×10⁷ pfu NDV-huIL-12 has a statistically significant increase in GEP score when compared to medium controls (P<0.0001), including conversion of a GEP-negative score (GEP score<−0.318) to a GEP-positive score (GEP score>−0.318). (FIGS. 40A-B).

### 6.3.2.7 Analytical Development and Characterization of NDV-huIL12

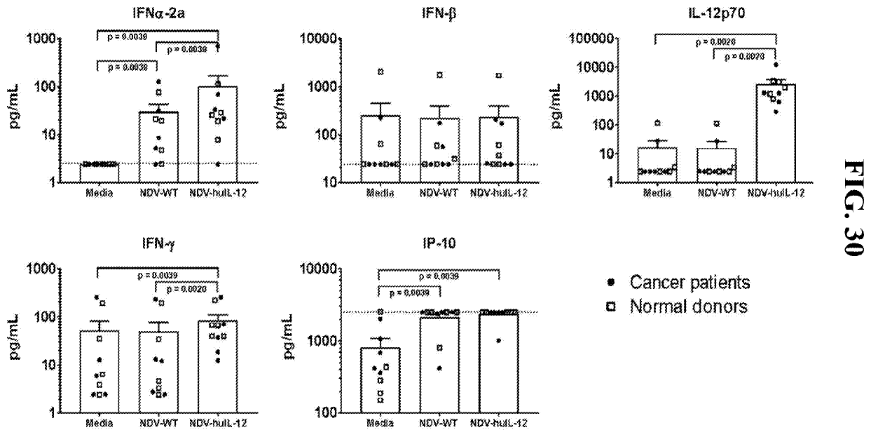

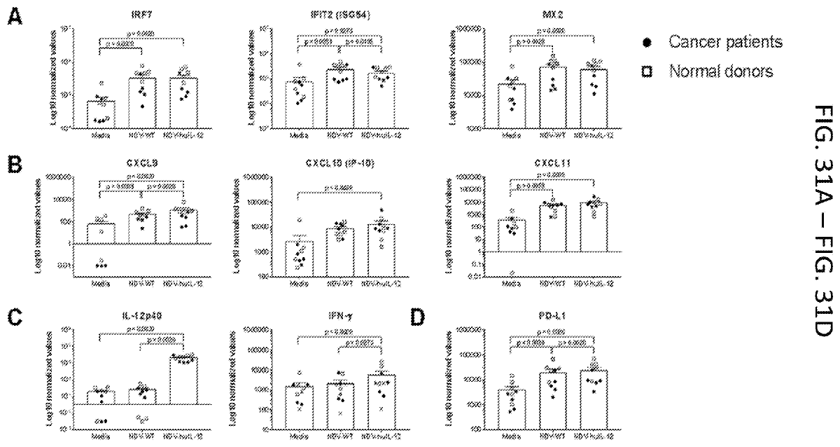

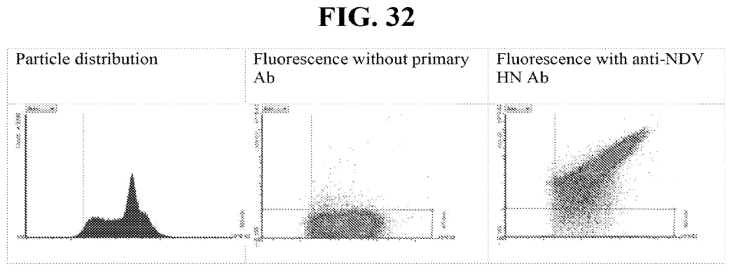

NDV-huIL12 was generated according to the methods provided in Section 6.3.1.1. NDV-huIL-12 can be quantified and characterized by flow virometry (FV, the use of a flow cytometer to analyze and quantitate individual particles). See, e.g., Vlasak et al., 2016, Vaccine 34:2321-2328 for a description of flow virometry. FV analysis showed that the majority of particles can be labeled with the mouse anti-NDV HN antibody 7B1 (ISMMS), suggesting that the majority of the particles are NDV-huIL-12 viruses (FIG. 32). NDV-huIL-12 shows pleiomorphic particle size distribution, consistent with the reported paramyxoviruses (Goff et al., 2012, J Virol. 86 (19): 10852-6).

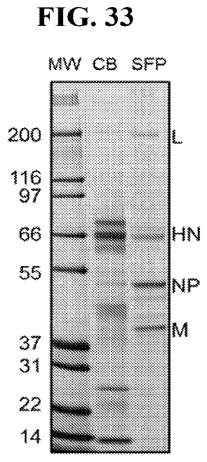

NDV contains six proteins: F, HN, NP, L, P, and M. SDS-PAGE analysis under reducing condition can separate 6 proteins (F1 (˜47 kDa), HN (˜63 kDa), NP (˜53 kDa), L (˜250 kDa), P (˜42 kDa), and M (˜40 kDa)) and serve as a qualitative purity assay. The purified NDV-huIL-12 was analyzed by SDS-PAGE (FIG. 33). It showed a similar protein banding pattern as other reported NDVs, suggesting that the majority proteins in the vaccine are viral proteins.

The production of human IL-12 by NDV-huIL-12 was confirmed by infecting Vero cells and quantitating IL-12 in the culture medium harvested 24 hours following infection. IL-12 was assayed by ELISA (FIG. 34). FIG. 34 shows the MOI-dependent huIL-12 expression compared to a reference batch of NDV-huIL-12.

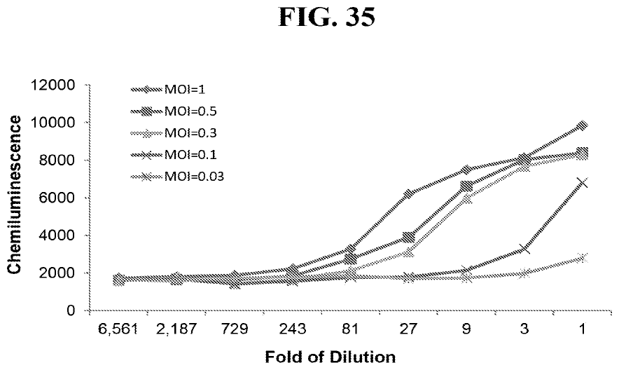

The activity of expressed huIL-12 was measured in a cell-based functional assay using PathHunter Dimerization assay from DiscoverX (FIG. 35). This assay detects huIL-12 induced receptor dimerization in engineered cells. Functional huIL-12 was detected in the supernatants of the NDV-huIL-12-infected vero cells at MOI of 0.1-1 (FIG. 35). The cell-based assay is optimized to characterize the purified viruses.

The cell-based assay is optimized to characterize the purified viruses.

### 6.4 Example 4: Clinical Study

The pre-clinical data described herein support the combination of intratumoral administration of NDV-huIL-12 with systemic intravenous co-administration of a blockade of immune checkpoint inhibitor PD-1 (see, e.g., Section 6.3). Combination of pembrolizumab and NDV-huIL-12 in patients with advanced solid tumors with accessible cutaneous and subcutaneous malignant lesions to permit intratumoral administration of NDV-huIL-12 is evaluated in clinical trials. These solid tumors include relapsed/refractory solid tumor types such as melanoma, squamous cell cancer of the head and neck (SSCHN), breast carcinoma, uterine cancer, gastric cancer, esophageal cancer, liver cancer, brain cancer, sarcoma with dermal metastases as well as other malignancies with accessible dermal/SC/nodal metastases. For indications where anti-PD-1/PD L1 therapy is approved, treatment of patients refractory or relapsed from these therapies as well as patients with no anticipated response to these agents are included in the study. Patients with relapsed, refractory and/or relapsed and refractory tumor types and tumors that are not usually responsive to anti-PD-1/PD-L1 but are amenable to intratumoral injection are also evaluated. Additional tumor types that are accessible by image-guidance, such as those with liver metastases are considered.

### 6.4.1 Production of NDV-huIL-12 and Purification of Virus

The NDV-huIL-12 may be produced in embryonated eggs or cell culture. In embryonated eggs, the NDV-hulL-12 may be propagated in the allantoic cavity of specific pathogen free embryonated chicken eggs. The NDV-hulL-12 may be purified using methods such as described in Section 6.3.1.20, supra. NDV-huIL-12 clinical drug product (DP) may be provided in a sterile solution for injection.

The NDV-huIL-12 may comprise a packaged genome comprising a nucleotide sequence encoding an IL-12 p40 subunit, said IL-12 p40 subunit comprising the amino acid sequence set forth in SEQ ID NO: 40, optionally wherein said nucleotide sequence comprises the nucleotide sequence set forth in SEQ ID NO: 54 or 64. The NDV-huIL-12 may comprise a packaged genome comprising a nucleotide sequence encoding an IL-12 p40 subunit, said IL-12 p40 subunit comprising an amino acid sequence set forth in SEQ ID NO: 38, optionally wherein said nucleotide sequence comprises the nucleotide sequence set forth in SEQ ID NO: 57 or 59. The NDV-huIL-12 may comprise a packaged genome comprising a nucleotide sequence encoding an IL-12 p40 subunit, said IL-12 p40 subunit comprising the amino acid sequence set forth in SEQ ID NO: 23, optionally wherein said nucleotide sequence comprises the nucleotide sequence set forth in SEQ ID NO: 27.

The NDV-huIL-12 may comprise a packaged genome comprising a nucleotide sequence encoding an IL-12 p35 subunit, said IL-12 p35 subunit comprising the amino acid sequence set forth in SEQ ID NO: 41, optionally wherein said nucleotide sequence comprises the nucleotide sequence set forth in SEQ ID NO:55 or 65. The NDV-huIL-12 may comprise a packaged genome comprising a nucleotide sequence encoding an IL-12 p35 subunit, said IL-12 p35 subunit comprising the amino acid sequence set forth in SEQ ID NO: 25, optionally wherein said nucleotide sequence comprises the nucleotide sequence set forth in SEQ ID NO:29.

The NDV huIL-12 may comprise a packaged genome comprising the nucleotide sequence set forth in SEQ ID NO: 51. Alternatively, the NDV huIL-12 may comprise a packaged genome comprising the nucleotide sequence set forth in SEQ ID NO: 52. Alternatively, NDV huIL-12 may comprise a packaged genome comprising the nucleotide sequence set forth in SEQ ID NO: 60.

The NDV-huIL-12 may comprise a packaged genome comprising a transgene encoding an amino acid sequence, wherein said amino acid sequence comprises the amino acid sequence set forth in SEQ ID NO: 42, optionally wherein said transgene comprises the nucleotide sequence set forth in SEQ ID NO: 53 or 66. The NDV-huIL-12 may comprise a packaged genome comprising a transgene encoding an amino acid sequence, wherein said amino acid sequence comprises the amino acid sequence set forth in SEQ ID NO: 43, optionally wherein said transgene comprises the nucleotide sequence set forth in SEQ ID NO: 63 or 68. The NDV-huIL-12 may comprise a packaged genome comprising a transgene encoding an amino acid sequence, wherein said amino acid sequence comprises the amino acid sequence set forth in SEQ ID NO: 22, optionally wherein said transgene comprises the nucleotide sequence set forth in SEQ ID NO: 26. The NDV-huIL-12 may comprise a packaged genome comprising a transgene encoding an amino acid sequence, wherein said amino acid sequence comprises the amino acid sequence set forth in SEQ ID NO: 39, optionally wherein said transgene comprises the nucleotide sequence set forth in SEQ ID NO: 61.

### 6.5 Example 5: rNDV-muIL12 Expression in BSRT7 Cells and Allantoic Fluid

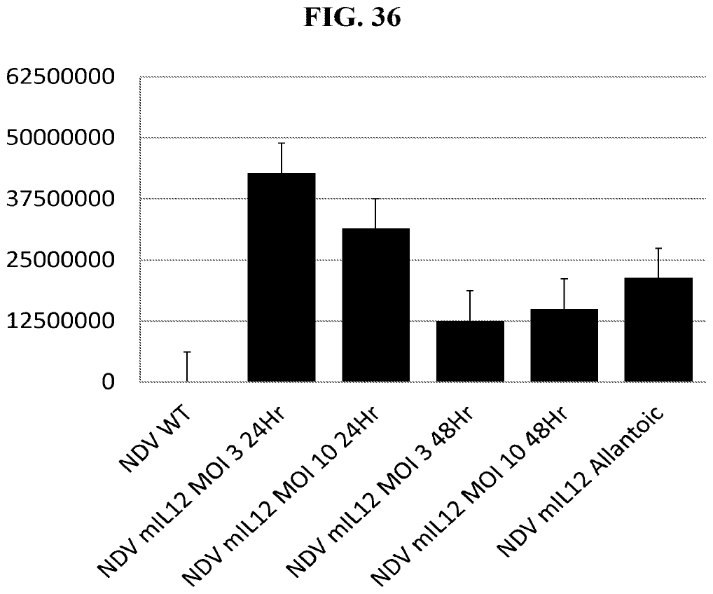

The expression of muIL-12 in BSRT7 cells infected with rNDV-muIL-12 was evaluated. rNDV-muIL-12 was generated as described in Section 6.3.1.1. BSRT7 cells were infected with rNDV-muIL-12 (also referred to as “NDV-muIL 12”) at a multiplicity of infection (“MOI”) of 3 or 10 and supernatants were collected at 24 or 48 hours post-infection. muIL-12 concentration in the collected supernatants was measured using the Mouse IL-12p70 Quantikine ELISA Kit (R&D Systems, Catalog No. M1270). BSRT7 cells infected with rNDV-muIL-12 under the tested conditions yielded detectable levels of muIL-12 (FIG. 36). Without being bound by any particular theory, it is hypothesized that the lower concentration of muIL-12 in cells infected at higher MOI as compared to cells infected at a lower MOI was due to a lower number of viable cells infected at the higher MOI producing muIL-12 as compared to the number of viable cells infected at the lower MOI producing muIL-12 (data not shown).

muIL-12 was also detected in the allantoic fluid of 10 day old embryonated eggs infected with 100 μl of rNDV-muIL-12 from a previous passage at 1:100000 dilution. The eggs were incubated for 2 days at 37 degrees Celsius, after which the allantoic fluid was collected from the eggs according to methods previously described (FIG. 36). muIL-12 concentration in the allantoic fluid was measured using the Mouse IL-12p70 Quantikine ELISA Kit (R&D Systems, Catalog No. M1270).

### 7. EMBODIMENTS

Provided herein are the following exemplary embodiments:

1. A method for treating cancer, comprising administering to a human subject in need thereof a first composition comprising a chimeric Newcastle disease virus (NDV) and a second composition comprising an antagonist of human PD-1 or a ligand thereof, wherein the chimeric NDV comprises a packaged genome comprising a transgene encoding human interleukin-12 (“IL-12”), wherein the transgene encodes a human IL-12 p40 subunit and a human IL-12 p35 subunit.

2. The method of embodiment 1, wherein the chimeric NDV comprises an NDV backbone which is lentogenic.

3. The method of embodiment 1 or 2, wherein the chimeric NDV comprises an NDV backbone of LaSota strain.

4. The method of embodiment 1 or 2, wherein the chimeric NDV comprises an NDV backbone of Hitchner B1 strain.

5. The method of embodiment 1 or 2, wherein the packaged genome comprises a nucleotide sequence encoding a mutated F protein and the mutated F protein is incorporated into the virion of the chimeric NDV, wherein the mutated F protein comprises a mutated cleavage site.

6. The method of embodiment 5, wherein the mutated cleavage site is ¹¹¹H-N-R-T-K-R/F-I¹¹⁸ (SEQ ID NO: 71).

7. The method of embodiment 1, wherein the chimeric NDV comprises an NDV backbone of a r73T-R116 virus.

8. The method of any one of embodiments 1 to 4, wherein the packaged genome comprises a nucleotide sequence encoding a mutated F protein with the amino acid mutation L289A, wherein the mutated F protein is incorporated into the virion of the chimeric NDV.

9. The method of any one of embodiments 1 to 8, wherein the IL-12 comprises the amino acid sequence set forth in SEQ ID NO:39.

10. The method of embodiment 9, wherein the transgene comprises the nucleotide sequence set forth in SEQ ID NO: 61.

11. The method of any one of embodiments 1 to 8, wherein the IL-12 comprises the amino acid sequence set forth in SEQ ID NO:22.

12. The method of embodiment 11, wherein the transgene comprises the nucleotide sequence set forth in SEQ ID NO: 26.

13. The method of any one of embodiments 1 to 8, wherein the IL-12 comprises the amino acid sequence set forth in SEQ ID NO:43.

14. The method of embodiment 13, wherein the transgene comprises the nucleotide sequence set forth in SEQ ID NO: 63.

15 The method of embodiment 13, wherein the transgene comprises the nucleotide sequence set forth in SEQ ID NO: 68.

16 The method of any one of embodiments 1 to 8, wherein the IL-12 comprises the amino acid sequence set forth in SEQ ID NO:42.

17 The method of embodiment 16, wherein the transgene comprises the nucleotide sequence set forth in SEQ ID NO: 53.

18 The method of embodiment 16, wherein the transgene comprises the nucleotide sequence set forth in SEQ ID NO: 66.

19. The method of any one of embodiments 1 to 8, wherein the IL-12 p40 subunit comprises the amino acid sequence set forth in SEQ ID NO: 38.

20. The method of any one of embodiments 1 to 8, wherein the IL-12 p40 subunit comprises the amino acid sequence set forth in SEQ ID NO: 23.

21. The method of embodiment 19 or 20, wherein the IL-12 p35 subunit comprises the amino acid sequence set forth in SEQ ID NO: 41.

22. The method of embodiment 19 or 20, wherein the IL-12 p35 subunit comprises the amino acid sequence set forth in SEQ ID NO: 25.

23. The method of any one of embodiments 1 to 22, wherein the transgene is inserted between two transcription units of the packaged genome.

24. The method of embodiment 23, wherein the packaged genome comprises a transcription unit for an NDV NP gene, a transcription unit for an NDV P gene, a transcription unit for an NDV M gene, a transcription unit for an NDV F gene, a transcription unit for an NDV HN gene, and a transcription unit for an NDV L gene.

25. The method of embodiment 24, wherein the two transcription units of the packaged genome are the transcription units for the NDV P gene and the NDV M gene.

26 The method of embodiment 1, wherein the packaged genome comprises the nucleotide sequence set forth in SEQ ID NO:51.

27 The method of embodiment 1, wherein the packaged genome comprises the nucleotide sequence set forth in SEQ ID NO:52.

28. The method of embodiment 1, wherein the packaged genome comprises the nucleotide sequence set forth in SEQ ID NO:60.

29 The method of any one of embodiments 1 to 28, wherein the antagonist of human PD-1 or a ligand thereof is an antibody that binds to human PD-1.

30 The method of embodiment 29, wherein the antibody blocks the interaction between human PD-1 and its ligands, PD-L1 and PD-L2.

31 The method of embodiment 29 or 30, wherein the antibody is pembrolizumab.

32. The method of embodiment 29 or 30, wherein the antibody is nivolumab or MEDI0680.

33. The method of embodiment 29 or 30, wherein the antibody comprises a variable light chain region (VLCR) complementarity determining region (CDR) 1 comprising the amino acid sequence RASKGVSTSGYSYLH (SEQ ID NO: 1), a VLCR CDR2 comprising the amino acid sequence LASYLES (SEQ ID NO: 2), a VLCR CDR3 comprising the amino acid sequence QHSRDLPLT (SEQ ID NO: 3), a variable heavy chain region (VHCR) CDR1 comprising the amino acid sequence NYYMY (SEQ ID NO: 6), a VHCR CDR2 comprising the amino acid sequence GINPSNGGTNFNEKFKN (SEQ ID NO: 7), and a VHCR CDR3 comprising the amino acid sequence RDYRFDMGFDY (SEQ ID NO: 8).

34 The method of embodiment 29 or 30, wherein the antibody comprises:
(a) a VLCR comprising the amino acid sequence EIVLTQSPATLSLSPGERATLSCRASKGVSTSGYSYLHWYQQKPGQAPRILLIYLAS YLESGVPARFSGSGSGTDFTLTISSLEPEDFAVYYCQHSRDLPLTFGGGTKVEIK (SEQ ID NO: 4); and
(b) a VHCR comprising the amino acid sequence QVQLVQSGVEVKKPGASVKVSCKASGYTFTNYYMYWVRQAPGQGLEWMGGI NPSNGGTNFNEKFKNRVTLTTDSSTTTAYMELKSLQFDDTAVYYCARRDYRFD MGFDYWGQGTTVTVSS (SEQ ID NO: 9).

35 The method of embodiment 29 or 30, wherein the antibody comprises:
(a) a light chain comprising the amino acid sequence EIVLTQSPATLSLSPGERATLSCRASKGVSTSGYSYLHWYQQKPGQAPRILLIYLAS YLESGVPARFSGSGSGTDFTLTISSLEPEDFAVYYCQHSRDLPLTFGGGTKVEIKR TVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 5); and
(b) a heavy chain comprising the amino acid sequence QVQLVQSGVEVKKPGASVKVSCKASGYTFTNYYMYWVRQAPGQGLEWMGGI NPSNGGTNFNEKFKNRVTLTTDSSTTTAYMELKSLQFDDTAVYYCARRDYRFD MGFDYWGQGTTVTVSSASTKGPSVFPLAPCSRSTSESTAALGCLVKDYFPEPVTV SWNSGALTSGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCVVVDVSQED PEVQFNWYVDGVEVHNAKTKPREEQFNSTYRVVSVLTVLHQDWLNGKEYKCK VSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEAL HNHYTQKSLSLSLGK (SEQ ID NO: 10).

36. The method of embodiment 29 or 30, wherein the antibody comprises a VLCR CDR1 comprising the amino acid sequence RASQSVSSYLA (SEQ ID NO: 11), a VLCR CDR2 comprising the amino acid sequence DASNRAT (SEQ ID NO: 12), a VLCR CDR3 comprising the amino acid sequence QQSSNWPRT (SEQ ID NO: 13), a VHCR CDR1 comprising the amino acid sequence NSGMH (SEQ ID NO: 16), a VHCR CDR2 comprising the amino acid sequence VIWYDGSKRYYADSVKG (SEQ ID NO: 17), and a VHCR CDR3 comprising the amino acid sequence NDDY (SEQ ID NO: 18).

37. The method of embodiment 29 or 30, wherein the antibody comprises:
(a) a VLCR comprising the amino acid sequence EIVLTQSPATLSLSPGERATLSCRASQSVSSYLAWYQQKPGQAPRLLIYDASNRA TGIPARFSGSGSGTDFTLTISSLEPEDFAVYYCQQSSNWPRTFGQGTKVEIK (SEQ ID NO: 14); and
(b) a VHCR comprising the amino acid sequence QVQIVESGGGVVQPGRSLRLDCKASGITFSNSGMHWVRQAPGKGLEWVAVIW YDGSKRYYADSVKGRFTISRDNSKNTILFLQMNSLRAEDTAVYYCATNDDYWGQ GTLVTVSS (SEQ ID NO: 19).

38 The method of embodiment 29 or 30, wherein the antibody comprises:
(a) a light chain comprising the amino acid sequence EIVLTQSPATLSLSPGERATLSCRASQSVSSYLAWYQQKPGQAPRLLIYDASNRA TGIPARFSGSGSGTDFTLTISSLEPEDFAVYYCQQSSNWPRTFGQGTKVEIKRTVA APSVFIFPPSDEQLKSGTASVVCLLNNFYPREAKVQWKVDNALQSGNSQESVTE QDSKDSTYSLSSTLTLSKADYEKHKVYACEVTHQGLSSPVTKSENRGEC (SEQ ID NO: 15); and
(b) a heavy chain comprising the amino acid sequence QVQLVESGGGVVQPGRSLRLDCKASGITFSNSGMHWVRQAPGKGLEWVAVIW YDGSKRYYADSVKGRFTISRDNSKNTLFLQMNSLRAEDTAVYYCATNDDYWGQ GTLVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKY GPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSS IEKTISKAKGQPREPQVYTLPPSQEEMTKNQVSLTCLVKGFYPSDIAVEWESNGQ PENNYKTTPPVLDSDGSFFLYSRLTVDKSRWQEGNVFSCSVMHEALHNHYTQKS LSLSLGK (SEQ ID NO: 20).

39 The method of any one of embodiments 1 to 28, wherein the antagonist of human PD-1 or a ligand thereof is an antibody that binds to human PD-L1.

40. The method of embodiment 39, wherein the antibody is durvalumab, avelumab, bms-936559, or atezolizumab.

41 The method of any one of embodiments 1 to 40, wherein the first composition is administered to the subject intratumorally or intra-nodally.

42. The method of embodiment 41, wherein the subject exhibits cutaneous or subcutaneous tumors or tumors within the lymph node.

43. The method of any one of embodiments 1 to 42, wherein the second composition is administered to the subject intravenously.

44. The method of any one of embodiments 1 to 42, wherein the second composition is administered to the subject subcutaneously.

45. The method of any one of embodiments 1 to 44, wherein the cancer is melanoma, kidney cancer, lung cancer, bladder cancer, ovarian cancer, hepatocellular carcinoma, pancreatic cancer, renal cancer, colorectal cancer, breast cancer, or head and neck cancer.

46. The method of any one of embodiments 1 to 44, wherein the cancer is non-small cell lung cancer, classical Hodgkin lymphoma, microsatellite instability-high cancer, melanoma, gastric cancer, urothelial carcinoma, or head and neck squamous cell cancer.

47. The method of any one of embodiments 1 to 44, wherein the cancer is diffuse large B-cell lymphoma or cervical cancer.

48 The method of any one of embodiments 1 to 44, wherein the cancer is melanoma, non-small cell lung cancer, head and neck cancer (HNSCC head and neck squamous cell carcinoma), Urothelial cancer, Triple negative breast cancer, gastric cancer, gastroesophageal junction adenocarcinoma, classical Hodgkin lymphoma, non-Hodgkin lymphoma, primary mediastinal B-cell lymphoma, mesothelioma, ovarian cancer, small cell lung cancer, esophageal cancer, nasopharyngeal cancer, anal cancer, biliary tract cancer, colorectal cancer, ER+/HER2-breast cancer, cervical cancer, thyroid cancer, salivary cancer, endometrial cancer, prostate cancer, glioblastoma, microsatellite instability-high (MSI-H) or mismatch repair deficient cancer (tissue agnostic), or tumors with high tumor mutational burden (tissue agnostic).

49. The method of embodiment 45, wherein the lung cancer is non-small cell lung cancer.

50. The method of embodiment 45, wherein the head and neck cancer is squamous cell carcinoma of the head and neck, the renal cancer is renal cell carcinoma, the colorectal cancer is colorectal carcinoma, or the breast cancer is breast carcinoma, triple negative breast cancer, or ER+/HER2-breast cancer.

51 The method of any one of embodiments 1 to 44, wherein the cancer is a solid tumor selected from the group consisting of melanoma, sarcoma, uterine cancer, gastric cancer, esophageal cancer, liver cancer, brain cancer, head and neck squamous cell carcinoma, and breast carcinoma.

52 The method of any one of embodiments 1 to 44, wherein the cancer is non-Hodgkin lymphoma or Hodgkin lymphoma.

53 The method of any one of embodiments 1 to 52, wherein the cancer is metastatic.

54. The method of any one of embodiments 1 to 51, wherein the cancer is unresectable.

55 The method of any one of embodiments 1 to 52, wherein the cancer comprises a dermal, subcutaneous, or nodal metastasis.

56 The method of any one of embodiments 1 to 55, wherein the cancer is refractory or relapsed, or both.

57 The method of any one of embodiments 1 to 56, wherein a biopsy of the cancer is PD-L1-positive.

58 The method of embodiment 57, wherein the biopsy has a tumor proportion score of at least 1%.

59 The method of embodiment 57, wherein the biopsy has a combined positive score of at least 1.

60. The method of any one of embodiments 1 to 56, wherein a biopsy of the cancer is PD-L1-negative.

61. The method of embodiment 60, wherein the biopsy has a tumor proportion score of less than 1%.

62. The method of embodiment 60, wherein the biopsy has a combined positive score of less than 1.

63 The method of any one of embodiments 1 to 62, wherein the subject is refractory to monotherapy treatment with an antibody that binds to PD-1 and blocks the interaction between PD-1 and its ligands, PD-L1 and PD-L2.

64. The method of embodiment 63, wherein the administration of the chimeric NDV induces IL-12p70 expression, IFN-γ expression or both IL-12p70 and IFN-γ expression.

65. The method of embodiment 63, wherein the administration of the chimeric NDV increases the Gene Expression Profiling (GEP) score of the 18-gene signature of Table 15.

66. The method of any one of embodiments 1 to 62, wherein the subject is refractory or unresponsive to monotherapy treatment with an antagonist of human PD-1 or a ligand thereof.

67. The method of embodiment 66, wherein the antagonist of PD-1 or a ligand thereof is nivolumab, AMP-224, MEDI0680, pembrolizumab, durvalumab, avelumab, bms-936559, or atezolizumab.

68. A chimeric NDV comprising a packaged genome, said packaged genome comprising a transgene encoding a human IL-12, wherein the IL-12 comprises the amino acid sequence set forth in SEQ ID NO:39.

69. The chimeric NDV of embodiment 68, wherein the transgene comprises the nucleotide sequence set forth in SEQ ID NO:61.

70 A chimeric NDV comprising a packaged genome, said packaged genome comprising a transgene encoding a human IL-12, wherein the IL-12 comprises the amino acid sequence set forth in SEQ ID NO:22.

71 The chimeric NDV of embodiment 70, wherein the transgene comprises the nucleotide sequence set forth in SEQ ID NO:26.

72. A chimeric NDV comprising a packaged genome, said packaged genome comprising a transgene encoding a human IL-12, wherein the IL-12 comprises the amino acid sequence set forth in SEQ ID NO:43.

73. The chimeric NDV of embodiment 72, wherein the transgene comprises the nucleotide sequence set forth in SEQ ID NO:63.

74 The chimeric NDV of embodiment 72, wherein the transgene comprises the nucleotide sequence set forth in SEQ ID NO:68.

75 A chimeric NDV comprising a packaged genome, said packaged genome comprising a transgene encoding a human IL-12, wherein the IL-12 comprises the amino acid sequence set forth in SEQ ID NO:42.

76. The chimeric NDV of embodiment 75, wherein the transgene comprises the nucleotide sequence set forth in SEQ ID NO:53.

77 The chimeric NDV of embodiment 75, wherein the transgene comprises the nucleotide sequence set forth in SEQ ID NO:66.

78. The chimeric NDV of any one of embodiments 68 to 77, wherein the chimeric NDV comprises an NDV backbone which is lentogenic.

79. The chimeric NDV of any one of embodiments 68 to 78, wherein the chimeric NDV comprises an NDV backbone of LaSota strain.

80. The chimeric NDV of any one of embodiments 68 to 78, wherein the chimeric NDV comprises an NDV backbone of Hitchner B1 strain.

81. The chimeric NDV of any one of embodiments 68 to 78, wherein the packaged genome comprises a nucleotide sequence encoding a mutated F protein and the mutated F protein is incorporated into the virion of the chimeric NDV, wherein the mutated F protein comprises a mutated cleavage site.

82. The chimeric NDV of embodiment 81, wherein the mutated cleavage site is ¹¹¹H-N-R-T-K-R/F-I¹¹⁸ (SEQ ID NO: 71).

83 The chimeric NDV of any one of embodiments 68 to 77, wherein the chimeric NDV comprises an NDV backbone of a r73T-R116 virus.

84 The chimeric NDV of any one of embodiments 68 to 80, wherein the packaged genome comprises a nucleotide sequence encoding a mutated F protein with the amino acid mutation L289A, wherein the mutated F protein is incorporated into the virion of the chimeric NDV.

85. The chimeric NDV of any one of embodiments 68 to 71, wherein the packaged genome comprises the nucleotide sequence set forth in SEQ ID NO:51.

86. The chimeric NDV of embodiment 72, 73, 75 or 76, wherein the packaged genome comprises the nucleotide sequence set forth in SEQ ID NO:52.

87. The chimeric NDV of embodiment 72, 74, 75 or 77, wherein the packaged genome comprises the nucleotide sequence set forth in SEQ ID NO:60.

88. The chimeric NDV of any one of embodiments 68 to 84, wherein the transgene is inserted between two transcription units of the packaged genome.

89. The chimeric NDV of embodiment 88, wherein the packaged genome comprises a transcription unit for an NDV NP gene, a transcription unit for an NDV P gene, a transcription unit for an NDV M gene, a transcription unit for an NDV F gene, a transcription unit for an NDV HN gene, and a transcription unit for an NDV L gene.

90. The chimeric NDV of embodiment 89, wherein the two transcription units of the packaged genome are the transcription units for the NDV P gene and the NDV M gene.

91. A chimeric NDV for use in a method for treating cancer in a human subject, wherein the chimeric NDV comprises a packaged genome comprising a transgene encoding human interleukin-12 (“IL-12”), wherein the transgene encodes a human IL-12 p40 subunit and a human IL-12 p35 subunit, and wherein the method further comprises administering an antagonist of human PD-1 or a ligand thereof.

92 The chimeric NDV of embodiment 91, wherein the chimeric NDV comprises an NDV backbone which is lentogenic.

93. The chimeric NDV of embodiment 91 or 92, wherein the chimeric NDV comprises an NDV backbone of Hitchner B1.

94. The chimeric NDV of embodiment 91 or 92, wherein the chimeric NDV comprises an NDV backbone of LaSota strain.

95 The chimeric NDV of embodiment 91 or 92, wherein the packaged genome comprises a nucleotide sequence encoding a mutated F protein and the mutated F protein is incorporated into the virion of the chimeric NDV, wherein the mutated F protein comprises a mutated cleavage site.

96. The chimeric NDV of embodiment 95, wherein the mutated cleavage site is ¹¹¹H-N-R-T-K-R/F-I¹¹⁸ (SEQ ID NO: 71).

97. The chimeric NDV of embodiment 91, wherein the chimeric NDV comprises an NDV backbone of a r73T-R116 virus.

98. The chimeric NDV of any one of embodiments 91 to 94, wherein the packaged genome comprises a nucleotide sequence encoding a mutated F protein with the amino acid mutation L289A, wherein the mutated F protein is incorporated into the virion of the chimeric NDV.

99. The chimeric NDV of any one of embodiments 91 to 98, wherein the IL-12 comprises the amino acid sequence set forth in SEQ ID NO:39.

100. The chimeric NDV of embodiment 99, wherein the transgene comprises the nucleotide sequence set forth in SEQ ID NO: 61.

101. The chimeric NDV of any one of embodiments 91 to 98, wherein the IL-12 comprises the amino acid sequence set forth in SEQ ID NO:22.

102. The chimeric NDV of embodiment 101, wherein the transgene comprises the nucleotide sequence set forth in SEQ ID NO: 26.

103. The chimeric NDV of any one of embodiments 91 to 98, wherein the IL-12 comprises the amino acid sequence set forth in SEQ ID NO:43.

104. The chimeric NDV of embodiment 103, wherein the transgene comprises the nucleotide sequence set forth in SEQ ID NO: 63.

105. The chimeric NDV of embodiment 103, wherein the transgene comprises the nucleotide sequence set forth in SEQ ID NO: 68.

106. The chimeric NDV of any one of embodiments 91 to 98, wherein the IL-12 comprises the amino acid sequence set forth in SEQ ID NO:42.

107. The chimeric NDV of embodiment 106, wherein the transgene comprises the nucleotide sequence set forth in SEQ ID NO: 53.

108. The chimeric NDV of embodiment 106, wherein the transgene comprises the nucleotide sequence set forth in SEQ ID NO: 66.

109. The chimeric NDV of any one of embodiments 91 to 98, wherein the IL-12 p40 subunit comprises the amino acid sequence set forth in SEQ ID NO: 38.

110. The chimeric NDV of any one of embodiments 91 to 98, wherein the IL-12 p40 subunit comprises the amino acid sequence set forth in SEQ ID NO: 40.

111. The chimeric NDV of embodiment 109 or 110, wherein the IL-12 p35 subunit comprises the amino acid sequence set forth in SEQ ID NO: 41.

112. The chimeric NDV of embodiment 109 or 110, wherein the IL-12 p35 subunit comprises the amino acid sequence set forth in SEQ ID NO: 25.

113. The chimeric NDV of any one of embodiments 91 to 112, wherein the transgene is inserted between two transcription units of the packaged genome.

114. The chimeric NDV of embodiment 113, wherein the packaged genome comprises a transcription unit for an NDV NP gene, a transcription unit for an NDV P gene, a transcription unit for an NDV M gene, a transcription unit for an NDV F gene, a transcription unit for an NDV HN gene, and a transcription unit for an NDV L gene.

115. The chimeric NDV of embodiment 114, wherein the two transcription units of the packaged genome are the transcription units for the NDV P gene and the NDV M gene.

116. The chimeric NDV of embodiment 91, wherein the packaged genome comprises the nucleotide sequence set forth in SEQ ID NO:51.

117. The chimeric NDV of embodiment 91, wherein the packaged genome comprises the nucleotide sequence set forth in SEQ ID NO:52.

118. The chimeric NDV of embodiment 91, wherein the packaged genome comprises the nucleotide sequence set forth in SEQ ID NO:60.

119. The chimeric NDV of any one of embodiments 91 to 118, wherein the antagonist of human PD-1 or a ligand thereof is an antibody that binds to PD-1.

120. The chimeric NDV of embodiment 119, wherein the antibody blocks the interaction between human PD-1 and its ligands, PD-L1 and PD-L2.

121. The chimeric NDV of embodiment 119 or 120, wherein the antibody is pembrolizumab.

122. The chimeric NDV of embodiment 119 or 120, wherein the antibody is nivolumab or MEDI0680.

123. The chimeric NDV of embodiment 119 or 120, wherein the antibody comprises a variable light chain region (VLCR) complementarity determining region (CDR) 1 comprising the amino acid sequence RASKGVSTSGYSYLH (SEQ ID NO: 1), a VLCR CDR2 comprising the amino acid sequence LASYLES (SEQ ID NO: 2), a VLCR CDR3 comprising the amino acid sequence QHSRDLPLT (SEQ ID NO: 3), a variable heavy chain region (VHCR) CDR1 comprising the amino acid sequence NYYMY (SEQ ID NO: 6), a VHCR CDR2 comprising the amino acid sequence GINPSNGGTNFNEKFKN (SEQ ID NO: 7), and a VHCR CDR3 comprising the amino acid sequence RDYRFDMGEDY (SEQ ID NO: 8).

124. The chimeric NDV of embodiment 119 or 120, wherein the antibody comprises:

(a) a VLCR comprising the amino acid sequence EIVLTQSPATLSLSPGERATLSCRASKGVSTSGYSYLHWYQQKPGQAPRLLIYLAS YLESGVPARFSGSGSGTDFTLTISSLEPEDFAVYYCQHSRDLPLTFGGGTKVEIK (SEQ ID NO: 4); and
(b) a VHCR comprising the amino acid sequence QVQLVQSGVEVKKPGASVKVSCKASGYTFTNYYMYWVRQAPGQGLEWMGGI NPSNGGTNFNEKFKNRVTLTTDSSTTTAYMELKSLQFDDTAVYYCARRDYRFD MGFDYWGQGTTVTVSS (SEQ ID NO: 9).

125. The chimeric NDV of embodiment 119 or 120, wherein the antibody comprises:
(a) a light chain comprising the amino acid sequence EIVLTQSPATLSLSPGERATLSCRASKGVSTSGYSYLHWYQQKPGQAPRLLIYLAS YLESGVPARFSGSGSGTDFTLTISSLEPEDFAVYYCQHSRDLPLTFGGGTKVEIKR TVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 5); and
(b) a heavy chain comprising the amino acid sequence QVQLVQSGVEVKKPGASVKVSCKASGYTFTNYYMYWVRQAPGQGLEWMGGI NPSNGGTNFNEKFKNRVTLTTDSSTTTAYMELKSLQFDDTAVYYCARRDYRFD MGFDYWGQGTTVTVSSASTKGPSVFPLAPCSRSTSESTAALGCLVKDYFPEPVTV SWNSGALTSGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCVVVDVSQED PEVQFNWYVDGVEVHNAKTKPREEQFNSTYRVVSVLTVLHQDWLNGKEYKCK VSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEAL HNHYTQKSLSLSLGK (SEQ ID NO: 10).

126. The chimeric NDV of embodiment 119 or 120, wherein the antibody comprises a VLCR CDR1 comprising the amino acid sequence RASQSVSSYLA (SEQ ID NO: 11), a VLCR CDR2 comprisng the amino acid sequence DASNRAT (SEQ ID NO: 12), a VLCR CDR3 comprising the amino acid sequence QQSSNWPRT (SEQ ID NO: 13), a VHCR CDR1 comprising the amino acid sequence NSGMH (SEQ ID NO: 16), a VHCR CDR2 comprising the amino acid sequence VIWYDGSKRYYADSVKG (SEQ ID NO: 17), and a VHCR CDR3 comprising the amino acid sequence NDDY (SEQ ID NO: 18).

127. The chimeric NDV of embodiment 119 or 120, wherein the antibody comprises:
(a) a VLCR comprising the amino acid sequence EIVLTQSPATLSLSPGERATLSCRASQSVSSYLAWYQQKPGQAPRLLIYDASNRA TGIPARFSGSGSGTDFTLTISSLEPEDFAVYYCQQSSNWPRTFGQGTKVEIK (SEQ ID NO: 14); and
(b) a VHCR comprising the amino acid sequence QVQLVESGGGVVQPGRSLRLDCKASGITFSNSGMHWVRQAPGKGLEWVAVIW YDGSKRYYADSVKGRFTISRDNSKNTLFLQMNSLRAEDTAVYYCATNDDYWGQ GTLVTVSS (SEQ ID NO: 19).

128. The chimeric NDV of embodiment 119 or 120, wherein the antibody comprises:
(a) a light chain comprising the amino acid sequence EIVLTQSPATLSLSPGERATLSCRASQSVSSYLAWYQQKPGQAPRLLIYDASNRA TGIPARFSGSGSGTDFTLTISSLEPEDFAVYYCQQSSNWPRTFGQGTKVEIKRTVA APSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGEC (SEQ ID NO: 15); and
(b) a heavy chain comprising the amino acid sequence QVQLVESGGGVVQPGRSLRLDCKASGITFSNSGMHWVRQAPGKGLEWVAVIW YDGSKRYYADSVKGRFTISRDNSKNTLFLQMNSLRAEDTAVYYCATNDDYWGQ GTLVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKY GPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSS IEKTISKAKGQPREPQVYTLPPSQEEMTKNQVSLTCLVKGFYPSDIAVEWESNGQ PENNYKTTPPVLDSDGSFFLYSRLTVDKSRWQEGNVFSCSVMHEALHNHYTQKS LSLSLGK (SEQ ID NO: 20).

129. The chimeric NDV of any one of embodiments 91 to 118, wherein the antagonist of human PD-1 or a ligand thereof is an antibody that binds to human PD-L1.

130. The chimeric NDV of embodiment 129, wherein the antibody is durvalumab, avelumab, bms-936559, or atezolizumab.

131. The chimeric NDV of any one of embodiments 91 to 130, wherein the chimeric NDV is administered to the subject intratumorally or intra-nodally.

132. The chimeric NDV of embodiment 131, wherein the subject exhibits cutaneous or subcutaneous tumors or tumors within the lymph node.

133. The chimeric NDV of any one of embodiments 91 to 131, wherein the antagonist of human PD-1 or a ligand thereof is administered to the subject intravenously.

134. The chimeric NDV of any one of embodiments 91 to 131, wherein the antagonist of human PD-1 or a ligand thereof is administered to the subject subcutaneously.

135. The chimeric NDV of any one of embodiments 91 to 134, wherein the cancer is melanoma, kidney cancer, lung cancer, bladder cancer, ovarian cancer, hepatocellular carcinoma, pancreatic cancer, renal cancer, colorectal cancer, breast cancer, or head and neck cancer.

136. The chimeric NDV of any one of embodiments 91 to 134, wherein the cancer is non-small cell lung cancer, classical Hodgkin lymphoma, microsatellite instability-high cancer, melanoma, gastric cancer, urothelial carcinoma, or head and neck squamous cell cancer.

137. The chimeric NDV of any one of embodiments 91 to 134, wherein the cancer is diffuse large B-cell lymphoma or cervical cancer.

138. The chimeric NDV of any one of embodiments 91 to 134, wherein the cancer is melanoma, non-small cell lung cancer, head and neck cancer (HNSCC head and neck squamous cell carcinoma), Urothelial cancer, Triple negative breast cancer, gastric cancer, gastroesophageal junction adenocarcinoma, classical Hodgkin lymphoma, non-Hodgkin lymphoma, primary mediastinal B-cell lymphoma, mesothelioma, ovarian cancer, small cell lung cancer, esophageal cancer, nasopharyngeal cancer, anal cancer, biliary tract cancer, colorectal cancer, ER+/HER2-breast cancer, cervical cancer, thyroid cancer, salivary cancer, endometrial cancer, prostate cancer, glioblastoma, microsatellite instability-high (MSI-H) or mismatch repair deficient cancer (tissue agnostic), or tumors with high tumor mutational burden (tissue agnostic)

139. The chimeric NDV of embodiment 135, wherein the lung cancer is non-small cell lung cancer.

140. The chimeric NDV of embodiment 135, wherein the head and neck cancer is squamous cell carcinoma of the head and neck, the renal cancer is renal cell carcinoma, the colorectal cancer is colorectal carcinoma, or the breast cancer is breast carcinoma, triple negative breast cancer, or ER+/HER2-breast cancer.

141. The chimeric NDV of any one of embodiments 91 to 134, wherein the cancer is a solid tumor selected from the group consisting of melanoma, sarcoma, uterine cancer, gastric cancer, esophageal cancer, liver cancer, brain cancer, head and neck squamous cell carcinoma, and breast carcinoma.

142. The chimeric NDV of any one of embodiments 91 to 134, wherein the cancer is Non-Hodgkin lymphoma or Hodgkin lymphoma.

143. The chimeric NDV of any one of embodiments 91 to 142, wherein the cancer is metastatic.

144. The chimeric NDV of any one of embodiments 91 to 141, wherein the cancer is unresectable.

145. The chimeric NDV of any one of embodiments 91 to 142, wherein the cancer comprises a dermal, subcutaneous, or nodal metastasis.

146. The chimeric NDV of any one of embodiments 91 to 145, wherein the cancer is refractory, relapsed or both.

147. The chimeric NDV of any one of embodiments 91 to 145, wherein a biopsy of the cancer is PD-L1-positive.

148. The chimeric NDV of embodiment 147, wherein the biopsy has a tumor proportion score of at least 1%.

149. The chimeric NDV of embodiment 147, wherein the biopsy has a combined positive score of at least 1.

150. The chimeric NDV of any one of embodiments 91 to 146, wherein a biopsy of the cancer is PD-L1-negative.

151. The chimeric NDV of embodiment 150, wherein the biopsy has a tumor proportion score of less than 1%.

152. The chimeric NDV of embodiment 150, wherein the biopsy has a combined positive score of less than 1.

153. The chimeric NDV of any one of embodiments 91 to 152, wherein the subject is refractory to montherapy treatment with an antibody that binds to PD-1 and blocks the interaction between PD-1 and its ligands, PD-L1 and PD-L2.

154. The chimeric NDV of embodiment 153, wherein the administration of the chimeric NDV induces IL-12p70 expression, IFN-γ expression, or both IL-12p70 and IFN-γ expression.

155. The chimeric NDV of embodiment 153, wherein the administration of the chimeric NDV increases the GEP score of the 18-gene signature of Table 15.

156. The chimeric NDV of any one of embodiments 91 to 152, wherein the subject is refractory or unresponsive to monotherapy treatment with an antagonist of PD-1 or a ligand thereof.

157. The chimeric NDV of embodiment 156, wherein the antagonist of PD-1 or a ligand thereof is nivolumab, AMP-224, MEDI0680, pembrolizumab, durvalumab, avelumab, bms-936559, or atezolizumab.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1.-42. (canceled)

43. A method for treating cancer in a human subject, comprising administering to the human subject a pharmaceutical composition of a chimeric NDV in an admixture with a pharmaceutical acceptable carrier, wherein the chimeric NDV comprises a packaged genome, said packaged genome comprising a transgene encoding a human IL-12, wherein the IL-12 comprises the amino acid sequence set forth in SEQ ID NO:39 or 22.

44. The method of claim 43, wherein the pharmaceutical composition is administered to the subject intratumorally.

45. The method of claim 43, wherein the cancer is melanoma, kidney cancer, lung cancer, bladder cancer, ovarian cancer, hepatocellular carcinoma, pancreatic cancer, renal cancer, colorectal cancer, breast cancer, head and neck cancer, non-Hodgkin lymphoma or Hodgkin lymphoma, or a solid tumor selected from the group consisting of melanoma, sarcoma, uterine cancer, gastric cancer, esophageal cancer, liver cancer, brain cancer, head and neck squamous cell carcinoma, or breast carcinoma.

46. The method of claim 43, wherein the cancer is melanoma, non-small cell lung cancer, head and neck cancer (HNSCC head and neck squamous cell carcinoma), Urothelial cancer, renal cell carcinoma, breast carcinoma, Triple negative breast cancer, gastric cancer, gastroesophageal junction adenocarcinoma, classical Hodgkin lymphoma, non-Hodgkin lymphoma, diffuse large B-cell lymphoma, primary mediastinal B-cell lymphoma, mesothelioma, ovarian cancer, small cell lung cancer, esophageal cancer, nasopharyngeal cancer, anal cancer, biliary tract cancer, colorectal cancer, colorectal carcinoma, ER+/HER2-breast cancer, cervical cancer, thyroid cancer, salivary cancer, endometrial cancer, prostate cancer, glioblastoma, microsatellite instability-high (MSI-H) or mismatch repair deficient cancer (tissue agnostic), or tumors with high tumor mutational burden (tissue agnostic).

47. The method of claim 43, wherein the cancer is unresectable.

48. A method for treating cancer in a human subject, comprising administering to the human subject a pharmaceutical composition of a chimeric NDV in an admixture with a pharmaceutical acceptable carrier, wherein the chimeric NDV comprises a packaged genome, said packaged genome comprising a transgene encoding a human IL-12, wherein the IL-12 comprises the amino acid sequence set forth in SEQ ID NO:43 or 42.

49. The method of claim 48, wherein the pharmaceutical composition is administered to the subject intratumorally.

50. The method of claim 48, wherein the cancer is melanoma, kidney cancer, lung cancer, bladder cancer, ovarian cancer, hepatocellular carcinoma, pancreatic cancer, renal cancer, colorectal cancer, breast cancer, head and neck cancer, non-Hodgkin lymphoma or Hodgkin lymphoma, or a solid tumor selected from the group consisting of melanoma, sarcoma, uterine cancer, gastric cancer, esophageal cancer, liver cancer, brain cancer, head and neck squamous cell carcinoma, or breast carcinoma.

51. The method of claim 48, wherein the cancer is melanoma, non-small cell lung cancer, head and neck cancer (HNSCC head and neck squamous cell carcinoma), Urothelial cancer, renal cell carcinoma, breast carcinoma, Triple negative breast cancer, gastric cancer, gastroesophageal junction adenocarcinoma, classical Hodgkin lymphoma, non-Hodgkin lymphoma, diffuse large B-cell lymphoma, primary mediastinal B-cell lymphoma, mesothelioma, ovarian cancer, small cell lung cancer, esophageal cancer, nasopharyngeal cancer, anal cancer, biliary tract cancer, colorectal cancer, colorectal carcinoma, ER+/HER2-breast cancer, cervical cancer, thyroid cancer, salivary cancer, endometrial cancer, prostate cancer, glioblastoma, microsatellite instability-high (MSI-H) or mismatch repair deficient cancer (tissue agnostic), or tumors with high tumor mutational burden (tissue agnostic).

52. The method of claim 48, wherein the cancer is unresectable.
